SEQUENCE LISTING (980 sequences)

SEQ ID NO: 1 - protein, 17 aa, Homo sapiens
SSCMGGMNQRPILTIIT

SEQ ID NO: 2 - DNA, 51 bp, Homo sapiens
agttcctgcatgggcggcatgaaccagaggcccatcctcaccatcatcaca

SEQ ID NO: 3 - protein, 17 aa, Homo sapiens
SSCMGGMNWRPILTIIT

SEQ ID NO: 4 - DNA, 51 bp, Homo sapiens
agttcctgcatgggcggcatgaattggaggcccatcctcaccatcatcaca

SEQ ID NO: 5 - protein, 17 aa, Homo sapiens
LGRNSFEVCVCACPGRD

SEQ ID NO: 6 - DNA, 51 bp, Homo sapiens
ctgggacggaacagctttgaggtgtgtgtttgtgcctgtcctgggagagac

SEQ ID NO: 7 - protein, 17 aa, Homo sapiens
LGRNSFEVLVCACPGRD

SEQ ID NO: 8 - DNA, 51 bp, Homo sapiens
ctgggacggaacagctttgaggtgcttgtttgtgcctgtcctgggagagac

SEQ ID NO: 9 - protein, 17 aa, Homo sapiens
MCNSSCMGSMNRRPILT

SEQ ID NO: 10 - DNA, 51 bp, Homo sapiens
atgtgtaacagttcctgcatgggcagcatgaaccggaggcccatcctcacc

SEQ ID NO: 11 - protein, 17 aa, Homo sapiens
FRHSVVVPCEPPEVGSD

SEQ ID NO: 12 - DNA, 51 bp, Homo sapiens
tttcgacatagtgtggtggtgccctgtgagccgcctgaggttggctctgac

SEQ ID NO: 13 - protein, 17 aa, Homo sapiens
VCACPGRDWRTEEENLR

SEQ ID NO: 14 - DNA, 51 bp, Homo sapiens
gtttgtgcctgtcctgggagagattggcgcacagaggaagagaatctccgc

SEQ ID NO: 15 - protein, 17 aa, Homo sapiens
FVQGKDWGCKKFIRRDF

SEQ ID NO: 16 - DNA, 51 bp, Homo sapiens
tttgtgcaaggcaaagactggggatgcaagaaattcatccgtagagatttt

SEQ ID NO: 17 - protein, 17 aa, Homo sapiens
FVQGKDWGIKKFIRRDF

SEQ ID NO: 18 - DNA, 51 bp, Homo sapiens
tttgtgcaaggcaaagactggggaatcaagaaattcatccgtagagatttt

SEQ ID NO: 19 - protein, 17 aa, Homo sapiens
FVQGKDWGLKKFIRRDF

SEQ ID NO: 20 - DNA, 51 bp, Homo sapiens
tttgtgcaaggcaaagactggggattaaagaaattcatccgtagagatttt

SEQ ID NO: 21 - protein, 17 aa, Homo sapiens
FVQGKDWGSKKFIRRDF

SEQ ID NO: 22 - DNA, 51 bp, Homo sapiens
tttgtgcaaggcaaagactggggatccaagaaattcatccgtagagatttt

SEQ ID NO: 23 - protein, 17 aa, Homo sapiens
FVQGKDWGVKKFIRRDF

SEQ ID NO: 24 - DNA, 51 bp, Homo sapiens
tttgtgcaaggcaaagactggggagtcaagaaattcatccgtagagatttt

SEQ ID NO: 25 - protein, 17 aa, Homo sapiens
YRFVQGKDCGFKKFIRR

SEQ ID NO: 26 - DNA, 51 bp, Homo sapiens
tataggtttgtgcaaggcaaagactgtggattcaagaaattcatccgtaga

SEQ ID NO: 27 - protein, 17 aa, Homo sapiens
YRFVQGKDGGFKKFIRR

SEQ ID NO: 28 - DNA, 51 bp, Homo sapiens
tataggtttgtgcaaggcaaagacgggggattcaagaaattcatccgtaga

SEQ ID NO: 29 - protein, 17 aa, Homo sapiens
YRFVQGKDLGFKKFIRR

SEQ ID NO: 30 - DNA, 51 bp, Homo sapiens
tataggtttgtgcaaggcaaagactggggattcaagaaattcatccgtaga

SEQ ID NO: 31 - protein, 17 aa, Homo sapiens
YRFVQGKDRGFKKFIRR

SEQ ID NO: 32 - DNA, 51 bp, Homo sapiens
tataggtttgtgcaaggcaaagaccggggattcaagaaattcatccgtaga

SEQ ID NO: 33 - protein, 17 aa, Homo sapiens
YRFVQGKDSGFKKFIRR

SEQ ID NO: 34 - DNA, 51 bp, Homo sapiens
tataggtttgtgcaaggcaaagactcgggattcaagaaattcatccgtaga

SEQ ID NO: 35 - protein, 17 aa, Homo sapiens
YPVGYEATHIYWSLRTN

SEQ ID NO: 36 - DNA, 51 bp, Homo sapiens
tatcccgtgggctacgaggccacgcacatctattggagcctccgcaccaac

SEQ ID NO: 37 - protein, 17 aa, Homo sapiens
MFENGCYLCRQKRFKCE

SEQ ID NO: 38 - DNA, 51 bp, Homo sapiens
atgttcgagaacggctgctacttgtgccgccagaagcgcttcaagtgcgag

SEQ ID NO: 39 - protein, 17 aa, Homo sapiens
GKGSYWTLQPDSGNMFE

SEQ ID NO: 40 - DNA, 51 bp, Homo sapiens
ggcaagggctcctactggacgctgcagccggactccggcaacatgttcgag

SEQ ID NO: 41 - protein, 17 aa, Homo sapiens
GKGSYWTLLPDSGNMFE

SEQ ID NO: 42 - DNA, 51 bp, Homo sapiens
ggcaagggctcctactggacgctgctcccggactccggcaacatgttcgag

SEQ ID NO: 43 - protein, 17 aa, Homo sapiens
GKGSYWTLNPDSGNMFE

SEQ ID NO: 44 - DNA, 51 bp, Homo sapiens
ggcaagggctcctactggacgctgaacccggactccggcaacatgttcgag

SEQ ID NO: 45 - protein, 17 aa, Homo sapiens
GKGSYWTLYPDSGNMFE

SEQ ID NO: 46 - DNA, 51 bp, Homo sapiens
ggcaagggctcctactggacgctgtacccggactccggcaacatgttcgag

SEQ ID NO: 47 - protein, 17 aa, Homo sapiens
CYLRRQKRCKCEKQPGA

SEQ ID NO: 48 - DNA, 51 bp, Homo sapiens
tgctacttgcgccgccagaagcgctgcaagtgcgagaagcagccgggggcc

SEQ ID NO: 49 - protein, 17 aa, Homo sapiens
CYLRRQKRSKCEKQPGA

SEQ ID NO: 50 - DNA, 51 bp, Homo sapiens
tgctacttgcgccgccagaagcgctccaagtgcgagaagcagccgggggcc

SEQ ID NO: 51 - protein, 17 aa, Homo sapiens
IRHSLSFNGCFVKVARS

SEQ ID NO: 52 - DNA, 51 bp, Homo sapiens
atccgccactcgctgtccttcaatggctgcttcgtcaaggtggcacgctcc

SEQ ID NO: 53 - protein, 17 aa, Homo sapiens
IRHSLSFNNCFVKVARS

SEQ ID NO: 54 - DNA, 51 bp, Homo sapiens
atccgccactcgctgtccttcaataactgcttcgtcaaggtggcacgctcc

SEQ ID NO: 55 - protein, 17 aa, Homo sapiens
QQRWQNSICHSLSFNDC

SEQ ID NO: 56 - DNA, 51 bp, Homo sapiens
cagcagcgctggcagaactccatctgccactcgctgtccttcaatgactgc

SEQ ID NO: 57 - protein, 17 aa, Homo sapiens
QQRWQNSISHSLSFNDC

SEQ ID NO: 58 - DNA, 51 bp, Homo sapiens
cagcagcgctggcagaactccatcagccactcgctgtccttcaatgactgc

SEQ ID NO: 59 - protein, 17 aa, Homo sapiens
TLHPDSGNKFENGCYLR

SEQ ID NO: 60 - DNA, 51 bp, Homo sapiens
acgctgcacccggactccggcaacaagttcgagaacggctgctacttgcgc

SEQ ID NO: 61 - protein, 17 aa, Homo sapiens
TLHPDSGNRFENGCYLR

SEQ ID NO: 62 - DNA, 51 bp, Homo sapiens
acgctgcacccggactccggcaacaggttcgagaacggctgctacttgcgc

SEQ ID NO: 63 - protein, 17 aa, Homo sapiens
CHKKNFLHWDIKCSNIL

SEQ ID NO: 64 - DNA, 51 bp, Homo sapiens
tgtcacaaaaagaatttcctgcattgggatattaagtgttctaacattttg

SEQ ID NO: 65 - protein, 17 aa, Homo sapiens
IHCKAGKGQTGVMICAY

SEQ ID NO: 66 - DNA, 51 bp, Homo sapiens
attcactgtaaagctggaaagggacaaactggtgtaatgatatgtgcatat

SEQ ID NO: 67 - protein, 17 aa, Homo sapiens
WLSEDDNHFAAIHCKAG

SEQ ID NO: 68 - DNA, 51 bp, Homo sapiens
tggctaagtgaagatgacaatcattttgcagcaattcactgtaaagctgga

SEQ ID NO: 69 - protein, 17 aa, Homo sapiens
GLGDRHVQSILINEQSA

SEQ ID NO: 70 - DNA, 51 bp, Homo sapiens
ggacttggtgatagacatgtacagagtatcttgataaatgagcagtcagca

SEQ ID NO: 71 - protein, 17 aa, Homo sapiens
GLGDRHVQKILINEQSA

SEQ ID NO: 72 - DNA, 51 bp, Homo sapiens
ggacttggtgatagacatgtacagaaaatcttgataaatgagcagtcagca

SEQ ID NO: 73 - protein, 17 aa, Homo sapiens
NINIGPGDSEWFVVPEG

SEQ ID NO: 74 - DNA, 51 bp, Homo sapiens
aacataaatattggcccaggtgactctgaatggtttgttgttcctgaaggt

SEQ ID NO: 75 - protein, 17 aa, Homo sapiens
NINIGPGDYEWFVVPEG

SEQ ID NO: 76 - DNA, 51 bp, Homo sapiens
aacataaatattggcccaggtgactatgaatggtttgttgttcctgaaggt

SEQ ID NO: 77 - protein, 17 aa, Homo sapiens
FMKQMNDARHGGWTTKM

SEQ ID NO: 78 - DNA, 51 bp, Homo sapiens
ttcatgaaacaaatgaatgatgcacgtcatggtggctggacaacaaaaatg

SEQ ID NO: 79 - protein, 17 aa, Homo sapiens
RDPLSEITKQEKDFLWS

SEQ ID NO: 80 - DNA, 51 bp, Homo sapiens
cgagatcctctctctgaaatcactaagcaggagaaagattttctatggagt

SEQ ID NO: 81 - protein, 17 aa, Homo sapiens
RDPLSEITGQEKDFLWS

SEQ ID NO: 82 - DNA, 51 bp, Homo sapiens
cgagatcctctctctgaaatcactgggcaggagaaagattttctatggagt

SEQ ID NO: 83 - protein, 17 aa, Homo sapiens
RDPLSEITAQEKDFLWS

SEQ ID NO: 84 - DNA, 51 bp, Homo sapiens
cgagatcctctctctgaaatcactgcgcaggagaaagattttctatggagt

SEQ ID NO: 85 - protein, 17 aa, Homo sapiens
SGIHSGATATAPSLSGK

SEQ ID NO: 86 - DNA, 51 bp, Homo sapiens
tctggaatccattctggtgccactgccacagctccttctctgagtggtaaa

SEQ ID NO: 87 - protein, 17 aa, Homo sapiens
HWQQQSYLASGIHSGAT

SEQ ID NO: 88 - DNA, 51 bp, Homo sapiens
cactggcagcaacagtcttacctggcctctggaatccattctggtgccact

SEQ ID NO: 89 - protein, 17 aa, Homo sapiens
HWQQQSYLHSGIHSGAT

SEQ ID NO: 90 - DNA, 51 bp, Homo sapiens
cactggcagcaacagtcttacctgcactctggaatccattctggtgccact

SEQ ID NO: 91 - protein, 17 aa, Homo sapiens
HWQQQSYLVSGIHSGAT

SEQ ID NO: 92 - DNA, 51 bp, Homo sapiens
cactggcagcaacagtcttacctggtctctggaatccattctggtgccact

SEQ ID NO: 93 - protein, 17 aa, Homo sapiens
HWQQQSYLYSGIHSGAT

SEQ ID NO: 94 - DNA, 51 bp, Homo sapiens
cactggcagcaacagtcttacctgtactctggaatccattctggtgccact

SEQ ID NO: 95 - protein, 17 aa, Homo sapiens
SYLDSGIHAGATTTAPS

SEQ ID NO: 96 - DNA, 51 bp, Homo sapiens
tcttacctggactctggaatccatgctggtgccactaccacagctccttct

SEQ ID NO: 97 - protein, 17 aa, Homo sapiens
SYLDSGIHCGATTTAPS

SEQ ID NO: 98 - DNA, 51 bp, Homo sapiens
tcttacctggactctggaatccattgtggtgccactaccacagctccttct

SEQ ID NO: 99 - protein, 17 aa, Homo sapiens
SYLDSGIHFGATTTAPS

SEQ ID NO: 100 - DNA, 51 bp, Homo sapiens
tcttacctggactctggaatccattttggtgccactaccacagctccttct

SEQ ID NO: 101 - protein, 17 aa, Homo sapiens
SYLDSGIHYGATTTAPS

SEQ ID NO: 102 - DNA, 51 bp, Homo sapiens
tcttacctggactctggaatccattatggtgccactaccacagctccttct

SEQ ID NO: 103 - protein, 17 aa, Homo sapiens
SGATTTAPCLSGKGNPE

SEQ ID NO: 104 - DNA, 51 bp, Homo sapiens
tctggtgccactaccacagctccttgtctgagtggtaaaggcaatcctgag

SEQ ID NO: 105 - protein, 17 aa, Homo sapiens
SGATTTAPFLSGKGNPE

SEQ ID NO: 106 - DNA, 51 bp, Homo sapiens
tctggtgccactaccacagctccttttctgagtggtaaaggcaatcctgag

SEQ ID NO: 107 - protein, 17 aa, Homo sapiens
SGATTTAPPLSGKGNPE

SEQ ID NO: 108 - DNA, 51 bp, Homo sapiens
tctggtgccactaccacagctcctcctctgagtggtaaaggcaatcctgag

SEQ ID NO: 109 - protein, 17 aa, Homo sapiens
YVPSEDYYKPSPYDDLT

SEQ ID NO: 110 - DNA, 51 bp, Homo sapiens
tacgtgcccagtgaggactactacaagccctcaccgtatgatgacctcacc

SEQ ID NO: 111 - protein, 17 aa, Homo sapiens
IRKRLKLCTDFKRTGMD

SEQ ID NO: 112 - DNA, 51 bp, Homo sapiens
atccggaagaggctaaagctctgcactgacttcaaacgcacagggatggat

SEQ ID NO: 113 - protein, 17 aa, Homo sapiens
VDGAVFAVFKAVFVLGD

SEQ ID NO: 114 - DNA, 51 bp, Homo sapiens
gtggatggagccgtgtttgctgttttcaaggctgtgtttgtacttggggat

SEQ ID NO: 115 - protein, 17 aa, Homo sapiens
IVDGAVFAGLKAVFVLG

SEQ ID NO: 116 - DNA, 51 bp, Homo sapiens
atcgtggatggagccgtgtttgctggtctcaaggctgtgtttgtacttggg

SEQ ID NO: 117 - protein, 17 aa, Homo sapiens
IVDGAVFALLKAVFVLG

SEQ ID NO: 118 - DNA, 51 bp, Homo sapiens
atcgtggatggagccgtgtttgctcttctcaaggctgtgtttgtacttggg

SEQ ID NO: 119 - protein, 17 aa, Homo sapiens
THTANERRWRGEMRDLF

SEQ ID NO: 120 - DNA, 51 bp, Homo sapiens
acacacactgccaatgagcggcggtggcgtggtgaaatgagggatctcttt

SEQ ID NO: 121 - protein, 17 aa, Homo sapiens
GRFSKVSSRAPMEGGEE

SEQ ID NO: 122 - DNA, 51 bp, Homo sapiens
gggaggttcagcaaggtgtctagtcgagctcccatggagggtggggaagaa

SEQ ID NO: 123 - protein, 17 aa, Homo sapiens
GKTEDLGCRYKLFSRVP

SEQ ID NO: 124 - DNA, 51 bp, Homo sapiens
ggaaagacagaagaccttggttgcaggtacaagttatttagtcgtgtgcca

SEQ ID NO: 125 - protein, 12 aa, Homo sapiens
MNHHQQQQQQKA

SEQ ID NO: 126 - DNA, 36 bp, Homo sapiens
atgaaccaccaccagcagcagcagcagcagaaagcg

SEQ ID NO: 127 - protein, 17 aa, Homo sapiens
HGLVDEQQEVRTISALA

SEQ ID NO: 128 - DNA, 51 bp, Homo sapiens
catggtcttgtggatgagcagcaggaagttcggaccatcagtgctttggcc

SEQ ID NO: 129 - protein, 17 aa, Homo sapiens
VCRHGDRCFRLHNKPTF

SEQ ID NO: 130 - DNA, 51 bp, Homo sapiens
gcatgtcgtcatggagacaggtgctttcggttgcacaataaaccgacgttt

SEQ ID NO: 131 - protein, 17 aa, Homo sapiens
SCTTPQCKCLLAKCCVD

SEQ ID NO: 132 - DNA, 51 bp, Homo sapiens
agttgtactacaccgcaatgcaaatgcctgcttgcaaaatgttgtgttgat

SEQ ID NO: 133 - protein, 17 aa, Homo sapiens
SILSKQVQHKPKTGRSL

SEQ ID NO: 134 - DNA, 51 bp, Homo sapiens
tccatattatctaaacaggttcaacataaaccaaaaactggtcgaagttta

SEQ ID NO: 135 - protein, 17 aa, Homo sapiens
QRIGSGSFATVYKGKWH

SEQ ID NO: 136 - DNA, 51 bp, Homo sapiens
caaagaattggatctggatcatttgcaacagtctacaagggaaagtggcat

SEQ ID NO: 137 - protein, 17 aa, Homo sapiens
GDFGLATVESRWSGSHQ

SEQ ID NO: 138 - DNA, 51 bp, Homo sapiens
ggtgattttggtctagctacagtggaatctcgatggagtgggtcccatcag

SEQ ID NO: 139 - protein, 17 aa, Homo sapiens
QANLRHLCCICGNSFRA

SEQ ID NO: 140 - DNA, 51 bp, Homo sapiens
caagccaaccttcgacatctctgctgcatctgtgggaattcttttagagct

SEQ ID NO: 141 - protein, 17 aa, Homo sapiens
QANLRHLCHICGNSFRA

SEQ ID NO: 142 - DNA, 51 bp, Homo sapiens
caagccaaccttcgacatctctgccacatctgtgggaattcttttagagct

SEQ ID NO: 143 - protein, 17 aa, Homo sapiens
DRCLKKVSKGVEQFEDI

SEQ ID NO: 144 - DNA, 51 bp, Homo sapiens
gatcgctgcctcaagaaggtgtccaagggcgtggagcagtttgaagatatt

SEQ ID NO: 145 - protein, 17 aa, Homo sapiens
IKTWVASNKIKDKRQLI

SEQ ID NO: 146 - DNA, 51 bp, Homo sapiens
atcaagacatgggtagcgtccaacaagatcaaggacaagaggcagcttata

SEQ ID NO: 147 - protein, 17 aa, Homo sapiens
QKFDEALRKSWTTKVNW

SEQ ID NO: 148 - DNA, 51 bp, Homo sapiens
caaaaatttgatgaggcgctcaggaaaagctggactactaaagtgaactgg

SEQ ID NO: 149 - protein, 17 aa, Homo sapiens
WVKPIIIGCHAYGDQYR

SEQ ID NO: 150 - DNA, 51 bp, Homo sapiens
tgggtaaaacctatcatcataggttgtcatgcttatggggatcaatacaga

SEQ ID NO: 151 - protein, 17 aa, Homo sapiens
WVKPIIIGGHAYGDQYR

SEQ ID NO: 152 - DNA, 51 bp, Homo sapiens
tgggtaaaacctatcatcataggtggtcatgcttatggggatcaatacaga

SEQ ID NO: 153 - protein, 17 aa, Homo sapiens
WVKPIIIGHHAYGDQYR

SEQ ID NO: 154 - DNA, 51 bp, Homo sapiens
tgggtaaaacctatcatcataggtcatcatgcttatggggatcaatacaga

SEQ ID NO: 155 - protein, 17 aa, Homo sapiens
YKLVVVGADGVGKSALT

SEQ ID NO: 156 - DNA, 51 bp, Homo sapiens
tataagctggtggtggtgggcgccgacggtgtgggcaagagtgcgctgacc

SEQ ID NO: 157 - protein, 17 aa, Homo sapiens
YKLVVVGARGVGKSALT

SEQ ID NO: 158 - DNA, 51 bp, Homo sapiens
tataagctggtggtggtgggcgcccgcggtgtgggcaagagtgcgctgacc

SEQ ID NO: 159 - protein, 17 aa, Homo sapiens
LDILDTAGKEEYSAMRD

SEQ ID NO: 160 - DNA, 51 bp, Homo sapiens
ttggacatcctggataccgccggaaaggaggagtacagcgccatgcgggac

SEQ ID NO: 161 - protein, 17 aa, Homo sapiens
LDILDTAGLEEYSAMRD

SEQ ID NO: 162 - DNA, 51 bp, Homo sapiens
ttggacatcctggataccgccggcctggaggagtacagcgccatgcgggac

SEQ ID NO: 163 - protein, 17 aa, Homo sapiens
LDILDTAGREEYSAMRD

SEQ ID NO: 164 - DNA, 51 bp, Homo sapiens
ttggacatcctggataccgccggccgggaggagtacagcgccatgcgggac

SEQ ID NO: 165 - protein, 17 aa, Homo sapiens
EGWLHKRGKYIKTWRPR

SEQ ID NO: 166 - DNA, 51 bp, Homo sapiens
gagggttggctgcacaaacgagggaagtacatcaagacctggcggccacgc

SEQ ID NO: 167 - protein, 17 aa, Homo sapiens
IARELHQFAFDLLIKSH

SEQ ID NO: 168 - DNA, 51 bp, Homo sapiens
attgcgagagagctgcatcagttcgcttttgacctgctaatcaagtcacac

SEQ ID NO: 169 - protein, 17 aa, Homo sapiens
IARELHQFGFDLLIKSH

SEQ ID NO: 170 - DNA, 51 bp, Homo sapiens
attgcgagagagctgcatcagttcggttttgacctgctaatcaagtcacac

SEQ ID NO: 171 - protein, 17 aa, Homo sapiens
QPDSFAALHSSLNELGE

SEQ ID NO: 172 - DNA, 51 bp, Homo sapiens
cagcccgactcctttgcagccttgcactctagcctcaatgaactgggagag

SEQ ID NO: 173 - protein, 17 aa, Homo sapiens
QMAVIQYSLMGLMVFAM

SEQ ID NO: 174 - DNA, 51 bp, Homo sapiens
cagatggctgtcattcagtactccttgatggggctcatggtgtttgccatg

SEQ ID NO: 175 - protein, 17 aa, Homo sapiens
QMAVIQYSFMGLMVFAM

SEQ ID NO: 176 - DNA, 51 bp, Homo sapiens
cagatggctgtcattcagtactcctttatggggctcatggtgtttgccatg

SEQ ID NO: 177 - protein, 68 aa, Homo sapiens
QNLQNGGGSRSSATLPGRRRRRWLRRRRQPISVAPAGPPRRPNQKPNPPGGARCVIMRPTWPGTSAFT

SEQ ID NO: 178 - DNA, 204 bp, Homo sapiens
cagaacctgcagaatggaggggggagcaggtcttcagccacactgccggggcggcggcggcggcggtggctgcggcggcggcggcagccaatatcagtagctcctgcggggccccctcgccgaccaaaccaaaaaccaaacccacctggcggtgcgaggtgtgtgattatgagaccaacgtggccaggaacctccgcattcaca

SEQ ID NO: 179 - protein, 33 aa, Homo sapiens
QNLQNGGGGAGLQPHCRGGGGGGGCGGGGSQYQ

SEQ ID NO: 180 - DNA, 102 bp, Homo sapiens
cagaacctgcagaatggaggggggggagcaggtcttcagccacactgccggggcggcggcggcggcggtggctgcggcggcggcggcagccaatatcagtag

SEQ ID NO: 181 - protein, 10 aa, Homo sapiens
NQEKEAEKNY

SEQ ID NO: 182 - DNA, 33 bp, Homo sapiens
aaccaagagaaagaggcagaaaaaaactattga

SEQ ID NO: 183 - protein, 40 aa, Homo sapiens
DAAVSPRGLQHRQGRGNLGWWQSPLRKVRVPKRRMVYHPS

SEQ ID NO: 184 - DNA, 123 bp, Homo sapiens
gatgctgctgtcagtcccagggggctgcagcacaggcaggggagagggaatctggggtggtggcagtctcccctgagaaaagtgagagtccccaaaaggaggatggtttatcatcccagttga

SEQ ID NO: 185 - protein, 33 aa, Homo sapiens
FVVSVVKKNRTCPFRLFVRRMLQFTGNKVLDRP

SEQ ID NO: 186 - DNA, 102 bp, Homo sapiens
tttgtcgtttctgttgtgaaaaaaaacaggacttgcccctttcgtctatttgtcagacgaatgttacaatttactggcaataaagttttggatagaccttaa

SEQ ID NO: 187 - protein, 9 aa, Homo sapiens
RSRRSAIKR

SEQ ID NO: 188 - DNA, 32 bp, Homo sapiens
agaagcagaagatcggctataaaaagataatg

SEQ ID NO: 189 - protein, 24 aa, Homo sapiens
SIIVQERERAERRVRRGQVMEIVD

SEQ ID NO: 190 - DNA, 75 bp, Homo sapiens
agtataattgtacaagagagagagagagcagagagaagggtcagaagaggccaagtgatggaaatagtggattaa

SEQ ID NO: 191 - protein, 34 aa, Homo sapiens
NNLVTEKKHRNVQCHDAVEENGQSSFITSPILHS

SEQ ID NO: 192 - DNA, 107 bp, Homo sapiens
aataacttggtcacagaaaaaaaacacagaaatgtgcaatgtcatgatgcagttgaggaaaatggccaatcatcctttattacatcgccaatattacacagctgaaa

SEQ ID NO: 193 - protein, 48 aa, Homo sapiens
HPQRKRRGVPPSPPLALGPRMQLCTQLARFFPITPPVWHILGPQRHTP

SEQ ID NO: 194 - DNA, 149 bp, Homo sapiens
cacccacagaggaaaaggcggggggtccctccgagcccacccctggctctcggccccaggatgcaactgtgcacccagcttgccagatttttccccattacacccccagtgtggcatatccttggtccccagaggcacaccccttgatc

SEQ ID NO: 195 - protein, 31 aa, Homo sapiens
ASRHHLWGATAGTPAPPPVPTCSPRTLRCLP

SEQ ID NO: 196 - DNA, 98 bp, Homo sapiens
gccagccggcaccatctgtggggggcaacagcgggcacccccgcaccaccccccgtgcccacctgttcacccaggaccctgcgatgcctcccctgacc

SEQ ID NO: 197 - protein, 13 aa, Homo sapiens
GPGEAGGPSPQGG

SEQ ID NO: 198 - DNA, 44 bp, Homo sapiens
gggcctggggaggctgggggcccctcaccccaaggcgggtgagc

SEQ ID NO: 199 - protein, 19 aa, Homo sapiens
GGGPSGSGEAPTSPSALRT

SEQ ID NO: 200 - DNA, 62 bp, Homo sapiens
ggcggggggcccagcggctcaggggaggctcccacttctccttcagccctgaggacatgaaa

SEQ ID NO: 201 - protein, 23 aa, Homo sapiens
GNPDVPPPVLFKADQSESSLSSG

SEQ ID NO: 202 - DNA, 72 bp, Homo sapiens
ggaaatccagatgtcccaccccccgtcctcttcaaggcagatcagagcgagagttctttgtcaagtgggtag

SEQ ID NO: 203 - protein, 10 aa, Homo sapiens
AFCYCGEKVP

SEQ ID NO: 204 - DNA, 35 bp, Homo sapiens
gctttttgttactgtggggaaaaagttccttagga

SEQ ID NO: 205 - protein, 16 aa, Homo sapiens
TLHPDSGNGCYLRRQK

SEQ ID NO: 206 - DNA, 50 bp, Homo sapiens
acgctgcacccggactccggcaacggctgctacttgcgccgccagaagcg

SEQ ID NO: 207 - protein, 16 aa, Homo sapiens
LHPDSGNMYGCYLRRQ

SEQ ID NO: 208 - DNA, 53 bp, Homo sapiens
acgctgcacccggactccggcaacatgtacggctgctacttgcgccgccagaa

SEQ ID NO: 209 - protein, 17 aa, Homo sapiens
LHPDSGNMCCYLRRQKR

SEQ ID NO: 210 - DNA, 51 bp, Homo sapiens
ctgcacccggactccggcaacatgtgctgctacttgcgccgccagaagcgc

SEQ ID NO: 211 - protein, 16 aa, Homo sapiens
CGASACDVSLIAMDSA

SEQ ID NO: 212 - DNA, 48 bp, Homo sapiens
tgcggggcctctgcctgtgatgtctccctcattgctatggacagtgct

SEQ ID NO: 213 - protein, 16 aa, Homo sapiens
TEYNQKLQVNQFSESK

SEQ ID NO: 214 - DNA, 48 bp, Homo sapiens
accgaatacaaccagaaattacaagtgaatcaatttagtgaatccaaa

SEQ ID NO: 215 - protein, 24 aa, Homo sapiens
TEISCCTLSSEENEYLPRPEWQLQ

SEQ ID NO: 216 - DNA, 72 bp, Homo sapiens
acagaaatttcatgttgcaccctgagcagtgaggagaatgaataccttccaagaccagagtggcagctccag

SEQ ID NO: 217 - protein, 16 aa, Homo sapiens
GLVSFGEHFCLPCALC

SEQ ID NO: 218 - DNA, 50 bp, Homo sapiens
gggctggtgtccttcggggagcacttttgtctgccctgcgccctctgcca

SEQ ID NO: 219 - protein, 16 aa, Homo sapiens
NSKMALNSEALSVVSE

SEQ ID NO: 220 - DNA, 48 bp, Homo sapiens
aacagcaagatggctttgaactcagaagccttatcagttgtgagtgag

SEQ ID NO: 221 - protein, 14 aa, Homo sapiens
CEERGAAGSLISCE

SEQ ID NO: 222 - DNA, 42 bp, Homo sapiens
tgtgaggagcgcggcgcggcaggaagccttatcagttgtgag

SEQ ID NO: 223 - protein, 69 aa, Homo sapiens
LWFQSSELSPTGAPWPSRRPTWRGTTVSPRTATSSARTCCGTKWPSSQEAALGLGSGLLRFSCGTAAIR

SEQ ID NO: 224 - DNA, 207 bp, Homo sapiens
ctgtggttccagagcagtgagctgtccccgacgggagcgccatggcccagccgccgcccgacgtggagggggacgactgtctccccgcgtaccgccacctcttctgcccggacctgctgcgggacaaagtggccttcatcacaggaggcggctctgggattgggttccggattgctgagattttcatgcggcacggctgccatacgg

SEQ ID NO: 225 - protein, 70 aa, Homo sapiens
WGMELAASRRFSWDHHSAGGPPRVPSVRSGAAQVQPKDPLPLRTLAGCLARTAHLRPGAESLPQPQLHCT

SEQ ID NO: 226 - DNA, 210 bp, Homo sapiens
tgggggatggagttggcagcgtctcggaggttctcctgggaccaccactccgccggggggccgcccagagtgccaagcgtccgatccggcgccgcccaagtgcagcccaaggacccgctcccgctccgcaccctggcaggctgcctagccaggactgcgcacctgcgccctggggcggagtccttaccccaaccccagcttcactgcaca

SEQ ID NO: 227 - protein, 28 aa, Homo sapiens
KEQILAVASLVSSQSIHPSWGQSPLSRI

SEQ ID NO: 228 - DNA, 84 bp, Homo sapiens
aaggaacagattttagctgtggccagtctcgtttcctctcagtccatccacccttcatggggccagagccctctctccagaatc

SEQ ID NO: 229 - protein, 14 aa, Homo sapiens
LELELSEGVCFRLR

SEQ ID NO: 230 - DNA, 42 bp, Homo sapiens
ctggagctggagctgtcggaaggagtctgcttcagattaaga

SEQ ID NO: 231 - protein, 17 aa, Homo sapiens
QQLRIFCAAMASNEDFS

SEQ ID NO: 232 - DNA, 52 bp, Homo sapiens
cagcagctaaggatattttgtgcagccatggcctccaacgaagatttctcca

SEQ ID NO: 233 - protein, 31 aa, Homo sapiens
DGFSGSLFAVVTRRCYFLKWRTIFPQSLMWL

SEQ ID NO: 234 - DNA, 93 bp, Homo sapiens
gacgggtttagcggcagcctcttcgcagttgtcaccagacgctgttacttcctaaaatggcggacaatcttcccacagagtttgatgtggtta

SEQ ID NO: 235 - protein, 16 aa, Homo sapiens
KMHFSLKEHPPPPCPP

SEQ ID NO: 236 - DNA, 48 bp, Homo sapiens
aaaatgcacttctccctcaaggagcacccaccgcccccttgcccgcct

SEQ ID NO: 237 - protein, 31 aa, Homo sapiens
DLRRVATYCAPLPSSWRPGTGTTIPPRMRSC

SEQ ID NO: 238 - DNA, 93 bp, Homo sapiens
gatctgcgccgggtcgccacatactgcgctcctttaccctcatcgtggaggcctgggactgggacaacgataccaccccgaatgaggagctgc

SEQ ID NO: 239 - protein, 48 aa, Homo sapiens
LQERMELLACGAERGAGGWGGGGGGGGGDRRGGGGSAPALADFAGGRG

SEQ ID NO: 240 - DNA, 144 bp, Homo sapiens
ttgcaggagcggatggagttgcttgcctgcggagccgagcgcggggccggcggctgggggggaggcggtggcggcggcggcggcgaccgaagaggaggaggaggaagcgcgccagctcttgcagactttgcaggcggccgaggg

SEQ ID NO: 241 - protein, 47 aa, Homo sapiens
LTFLDFIQVTLRVMSGSQMENGSSYFFKPFSWGLGVGLSAWLCVMLT

SEQ ID NO: 242 - DNA, 141 bp, Homo sapiens
ctgacgtttttagatttcatccaggtaacgttgagagtaatgtcaggatctcaaatggaaaacggaagttcctattttttcaagcccttttcatggggtctgggggtgggactctcggcctggctgtgtgtaatgttaact

SEQ ID NO: 243 - protein, 51 aa, Homo sapiens
FMIGELVGELCCQLTFRLPFLESLCQAVVTQALRFNPSFQEVCIYQDTDLM

SEQ ID NO: 244 - DNA, 153 bp, Homo sapiens
ttcatgattggagaacttgtaggtgagttgtgttgccaactcactttccgtttacctttcctcgagagtctttgtcaagctgtagttacacaggctttgaggtttaacccatcttttcaggaagtttgtatttatcaagacactgatctcatg

SEQ ID NO: 245 - protein, 16 aa, Homo sapiens
VAMMVPDRQVHYDFGL

SEQ ID NO: 246 - DNA, 48 bp, Homo sapiens
gttgctatgatggttcctgatagacaggttcattatgactttggattg

SEQ ID NO: 247 - protein, 24 aa, Homo sapiens
WCPLDLRLGSTGCLTCRHHQTSHE

SEQ ID NO: 248 - DNA, 72 bp, Homo sapiens
tggtgtccgctggatcttagactcggttccactggatgtctcacatgcagacatcatcaaacgtcacatgag

SEQ ID NO: 249 - protein, 25 aa, Homo sapiens
VVGRRHETAPQPLLVPDRAGGEGGA

SEQ ID NO: 250 - DNA, 75 bp, Homo sapiens
gtcgtgggaaggcgtcatgaaacagctcctcaacccctgctggtgcccgaccgagctggtggtgaagggggagca

SEQ ID NO: 251 - protein, 17 aa, Homo sapiens
DYWAQKEKISIPRTHLC

SEQ ID NO: 252 - DNA, 51 bp, Homo sapiens
gactactgggctcaaaaggagaagatcagcatccccagaacacacctgtgt

SEQ ID NO: 253 - protein, 18 aa, Homo sapiens
DYWAQKEKGSSSFLRPSC

SEQ ID NO: 254 - DNA, 54 bp, Homo sapiens
gactactgggctcaaaaggagaagggatcatcttcattcctgcgaccatcctgt

SEQ ID NO: 255 - protein, 14 aa, Homo sapiens
LVLGVLSGHSGSRL

SEQ ID NO: 256 - DNA, 42 bp, Homo sapiens
cttgtacttggtgtattgagcgggcacagtggctcacgccta

SEQ ID NO: 257 - protein, 25 aa, Homo sapiens
PVPTATPGVRSVTSPQGLGLFLKFI

SEQ ID NO: 258 - DNA, 75 bp, Homo sapiens
cctgtcccaactgctacacctggggtaagatcagtgactagtccccaggggctgggccttttccttaagtttatt

SEQ ID NO: 259 - protein, 28 aa, Homo sapiens
KENDVREVCDVYLQMQIFFHFKFRSYFH

SEQ ID NO: 260 - DNA, 84 bp, Homo sapiens
aaggaaaatgatgtccgtgaggtctgtgatgtgtatttacaaatgcagatcttcttccattttaagttcagaagttactttcat

SEQ ID NO: 261 - protein, 90 aa, Homo sapiens
VPFRELKNQRTAQGAPGIHHAASPVAANLCDPARHAQHTRIPCGAGQVRAGRGPEAGGGVLQPQRPAPEKPGCPCRRGQPRLHTVKMWRA

SEQ ID NO: 262 - DNA, 270 bp, Homo sapiens
gtgcccttccgggagctcaagaaccagagaacagcacaaggggctcctgggatccaccacgcggcttcccccgttgctgccaacctctgcgacccggcgagacacgcacagcacacacgcatcccctgcggcgctggccaagtgcgtgctggccgaggtcccgaagcaggtggtggagtactacagccacagaggcctgcccccgagaagcctgggtgtccctgccggagaggccagcccaggctgcacaccgtgaagatgtggagggcg

SEQ ID NO: 263 - protein, 22 aa, Homo sapiens
FARKMLEKVHRQHLQLSHNSQE

SEQ ID NO: 264 - DNA, 66 bp, Homo sapiens
tttgctagaaaaatgctggagaaggtacacagacaacacctacagctttcccacaatagccaggaa

SEQ ID NO: 265 - protein, 11 aa, Homo sapiens
KRSFAVTERII

SEQ ID NO: 266 - DNA, 33 bp, Homo sapiens
aagagaagttttgctgtcacggagaggatcatc

SEQ ID NO: 267 - protein, 17 aa, Homo sapiens
MFLRKEQQVGPHSFSML

SEQ ID NO: 268 - DNA, 51 bp, Homo sapiens
atgttccttagaaaggagcagcaggtgggtccccacagcttttctatgctt

SEQ ID NO: 269 - protein, 14 aa, Homo sapiens
VLRFLDLKVRYLHS

SEQ ID NO: 270 - DNA, 42 bp, Homo sapiens
gtgctgcgctttctggacttaaaggtgagatacctgcactct

SEQ ID NO: 271 - protein, 32 aa, Homo sapiens
GNTTLQQLGEASQAPSGSLIPLRLPLLWEVRG

SEQ ID NO: 272 - DNA, 96 bp, Homo sapiens
ggcaacaccaccctccagcagctgggtgaggcctcccaggcgccctcaggctccctcatccctctgaggctgcctctgctctgggaagtgaggggc

SEQ ID NO: 273 - protein, 25 aa, Homo sapiens
GLNLNTDRPGGYSYSIWWKNNAKNR

SEQ ID NO: 274 - DNA, 75 bp, Homo sapiens
ggactgaacttaaatactgatagaccaggtggttacagctattcaatttggtggaaaaacaatgccaagaacaga

SEQ ID NO: 275 - protein, 27 aa, Homo sapiens
WKFEMSYTVGGPPPHVHARPRHWKTDR

SEQ ID NO: 276 - DNA, 81 bp, Homo sapiens
tggaaattcgagatgagctacacggtgggtggcccgcctccccatgttcatgctagacccaggcattggaaaactgataga

SEQ ID NO: 277 - protein, 22 aa, Homo sapiens
QWQHYHRSGEAAGTPLWRPTRN

SEQ ID NO: 278 - DNA, 66 bp, Homo sapiens
cagtggcagcactaccaccggtcaggtgaggccgcagggactcccctctggagacccacaagaaac

SEQ ID NO: 279 - protein, 29 aa, Homo sapiens
KVLNEIDAVVTVPPSLSTSQIPQGCCIIL

SEQ ID NO: 280 - DNA, 87 bp, Homo sapiens
aaggtcctcaacgagatcgatgcggtagttaccgtccctccctccctgtctacctcccagataccgcagggctgctgcatcatattg

SEQ ID NO: 281 - protein, 18 aa, Homo sapiens
ANLKGTLQVRSGQAVSPR

SEQ ID NO: 282 - DNA, 54 bp, Homo sapiens
gccaatctgaaaggcaccctgcaggtgaggagtgggcaggcagtgagtccacgc

SEQ ID NO: 283 - protein, 70 aa, Homo sapiens
LQAAASGQGKQGVPCPWGCCAYAESPRALISGDAPSQVEREVPGPCLNTHSLSHRSPQLPGLPHPKQPSV

SEQ ID NO: 284 - DNA, 210 bp, Homo sapiens
ctccaggcggctgcctcgggccaaggcaagcagggcgtcccttgtccctggggttgctgtgcctacgctgagagtccccgggccctgatttcgggagatgctccatcacaggtggagcgggaggtgccgggcccctgcctcaacacgcattctctctcccacagatccccacagctcccaggccttccacaccccaagcagccttctgtt

SEQ ID NO: 285 - protein, 9 aa, Homo sapiens
KIQNKNCPD

SEQ ID NO: 286 - DNA, 27 bp, Homo sapiens
aaaattcagaataaaaattgtccagac

SEQ ID NO: 287 - protein, 37 aa, Homo sapiens
GEVELSEGGEGQRHLAFPWACSGPGWRGVCCAAVEPA

SEQ ID NO: 288 - DNA, 111 bp, Homo sapiens
ggcgaggtggagctctcagagggcggtgagggccagcggcaccttgcatttccctgggcctgctctgggccgggctggagaggggtgtgctgtgctgctgtggagcctgct

SEQ ID NO: 289 - protein, 9 aa, Homo sapiens
IEMKKLLKS

SEQ ID NO: 290 - DNA, 27 bp, Homo sapiens
attgaaatgaaaaaactgttaaaaagt

SEQ ID NO: 291 - protein, 70 aa, Homo sapiens
KMRAIQAEGGHGQACCGGAWGWAPGDGGPQGMLTHTLPTLGFQSAWTWRREDADRAWRTPKACASRRWSI

SEQ ID NO: 292 - DNA, 210 bp, Homo sapiens
aagatgcgggccatccaggccgagggtgggcacgggcaggcctgctgtggaggggcctggggatgggcaccgggggacgggggcccccaggggatgctcacgcatactctgcccaccctgggcttccagagcgcctggacatggagaagagaagatgcagacagagcctggaggactccgaaagcctgcgcatcaaggaggtggagcata

SEQ ID NO: 293 - protein, 74 aa, Homo sapiens
LLEPFRRGEPGPRGLLSGSSRGGEGPGRSIEAAPATPLPCCRKNPCRPQPSRFLPPRVLLVIILPKLDCPKLGF

SEQ ID NO: 294 - DNA, 222 bp, Homo sapiens
ctgctggagcccttccgccgcggtgagcccgggccccgcgggctgctctcgggaagttcccgcggaggggaggggcctggccgttcgatcgaggctgcacccgccacacctttgccctgttgccgcaagaacccttgtcggccccagccttccagatttttgcctcctagggtattgttagtgatcattcttcccaaactggattgtccaaaacttgggttc

SEQ ID NO: 295 - protein, 65 aa, Homo sapiens
PSGRRTKRLVTLRSGCAIQCWHPRAGPVPSALPHTERPPRLVRGAADPRTVTLGRSPAVMPRAPA

SEQ ID NO: 296 - DNA, 195 bp, Homo sapiens
ccctcggggcggagaaccaaacggttagttaccctgcgttctggctgcgccatacaatgctggcatcctcgtgccggcccagttccctcagcgcttcctcacacagagaggcccccaaggcttgtcaggggagcagcagatcccaggacagtgaccctgggacggagccctgcagtcatgcctcgggcccctgcg

SEQ ID NO: 297 - protein, 95 aa, Homo sapiens
CHLFLQPQVGTPPPHTASARAPSGPPHPHESCPAGRRPARAAQTCARRQHGLPGCEEAGTARVPSLHLHLHQAALGAGRGRGWGEACAQVPPSRG

SEQ ID NO: 298 - DNA, 285 bp, Homo sapiens
tgccacctcttcctgcagccccaggttggcaccccccccccccacactgccagtgctcgagcccccagtggtccaccccaccctcatgaaagttgccctgcagggcgaagacctgcgagagctgcgcagacatgtgcacgccgacagcacggacttcctggctgtgaagaggctggtacagcgcgtgttcccagcctgcacctgcacctgcaccaggccgccctcggagcaggaaggggccgtgggtggggagaggcctgtgcccaagtacccccctcaagaggc

SEQ ID NO: 299 - protein, 45 aa, Homo sapiens
KELKLEQQVGGQGLRGVGQGVRGGFVTLTTHTPFPSQEAAERESK

SEQ ID NO: 300 - DNA, 135 bp, Homo sapiens
aaggagctcaagctggagcagcaggtgggtgggcagggcttgagaggggtgggccaaggggtgcgtggcggcttcgtgaccctcactacccataccccgttcccctcccaggaagctgcagagcgggagtctaaa

SEQ ID NO: 301 - protein, 175 aa, Homo sapiens
GEISQEEVPPSRHLGVSWGAGVWAGLTLGASAPPNSSFPSGAELQPVVCCIRSDTRQPRPPDFPQHRGDPRLPQLSLGAENQTVSYPAFWLRHTMLASSCRPSSLSASSHREAPKACQGSSRSQDSDPGTEPCSHASGPCVTSTVSSPGLLPQRLLPLALTGLPVEEDGFEHAGA

SEQ ID NO: 302 - DNA, 525 bp, Homo sapiens
ggagaaatcagccaggaagaggtgcctccctcccgccacctgggcgtctcatggggtgctggggtgtgggcgggcctcaccctcggggcctctgcaccccctaactctagcttcccctcaggtgctgagctacagccagttgtgtgctgcattaggagtgacacaagacagccccgaccccccgactttcctcagcacaggggagatccacgccttcctcagctctccctcggggcggagaaccaaacggttagttaccctgcgttctggctgcgccatacaatgctggcatcctcgtgccggcccagttccctcagcgcttcctcacacagagaggcccccaaggcttgtcaggggagcagcagatcccaggacagtgaccctgggacggagccctgcagtcatgcctcgggcccctgcgtaacctccactgtctccagcccaggtctccttcctcagaggctattgcctctcgctctgactgggctccctgtggaggaagatggtttcgagcacgcgggagcc

SEQ ID NO: 303 - protein, 56 aa, Homo sapiens
DCMLSEEGGQARRGGSLCSLAAHTIASAARGRFLSRLSNFCAVVKASRGAPSCTWE

SEQ ID NO: 304 - DNA, 168 bp, Homo sapiens
gactgcatgctcagcgaggaaggtgggcaggcgcggcggggtggatccctctgctccttagctgcccacaccattgcctcggcagcccgaggtcgcttcctctccaggctctccaatttctgtgccgtagttaaagcgagcaggggcgccccttcctgcacctgggag

SEQ ID NO: 305 - protein, 43 aa, Homo sapiens
EAFQRAAGEGGPGRGGARRGARVLQSPFCRAGAGEWLGHQSLR

SEQ ID NO: 306 - DNA, 129 bp, Homo sapiens
gaggccttccagagggccgctggtgagggcggcccgggccgcggtggggcacggcgcggtgccagggtgttgcagagccccttttgcagggcaggagctggggagtggttaggacatcagtccctcagg

SEQ ID NO: 307 - protein, 35 aa, Homo sapiens
PEPRRLSPGEPRGRPRKGWGIWGLCGARVGPKAWR

SEQ ID NO: 308 - DNA, 105 bp, Homo sapiens
cctgagcccaggagactgagcccaggtgagcccagggggcgaccccggaagggctgggggatctggggtttgtgtggagcgcgggtggggcccaaggcttggcgg

SEQ ID NO: 309 - protein, 61 aa, Homo sapiens
VPFRELKNVSVLEGLRQGRLGGPCSCHCPRPSQARLTPVDVAGPFLCLGDPGLFPPVKSSI

SEQ ID NO: 310 - DNA, 183 bp, Homo sapiens
gtgcccttccgggagctcaagaacgtgagtgtcctggaggggctccgtcaaggccggcttgggggtccctgttcatgtcactgcccaagaccttcccaggccaggctcacgccagtggatgtggcaggtcccttcttgtgtctgggggatcctgggctgttccccccagtcaagagcagtatc

SEQ ID NO: 311 - protein, 98 aa, Homo sapiens
FVSLTAIQMASSATPWGRWPVATPTAACPRRRPSSLPTGGDSASKKPISRRAPWQPWACPGRSVNSAAPRAWCPPATTPRTQSPSRDLRPRCLSSWSS

SEQ ID NO: 312 - DNA, 294 bp, Homo sapiens
tttgtgagcctgactgccatccagatggcatcgtcggccactccctgggggaggtggcctgtggctacgccgacggctgcctgtcccaggaggaggccgtcctcgctgcctactggaggggacagtgcatcaaagaagcccatctcccgccgggcgccatggcagccgtgggcttgtcctgggaggagtgtaaacagcgctgccccccgggcgtggtgcccgcctgccacaactccaaggacacagtcaccatctcgggacctcaggccccggtgtttgagttcgtggagcagc

SEQ ID NO: 313 - protein, 109 aa, Homo sapiens
PVAIKPGTGPPNNSSIHGGSKRSENSYCRDLRGQLRAICCSSYSHDRHTTEERGSRGRHVWRIRRLHTSGLPCCCHSGPHPRRLPDILRLVTSTKTDHTNTTEGTLDYL

SEQ ID NO: 314 - DNA, 327 bp, Homo sapiens
ccagttgccattaaacctggtacagggccgcccaataactccagtatacacggtggctccaaacgttcagagaattcctactgccgggatctacggggccagttacgtgccatttgctgctccagctacagccacgatcgccacactacagaagaacgcggcagccgcggccgccatgtatggaggatacgcaggctacatacctcaggccttccctgctgctgccattcaggtccccatccccgacgtctaccagacatactgaggctggtgaccagcacgaagacagaccacacaaacaccactgaaggaacgcttgactattta

SEQ ID NO: 315 - protein, 40 aa, Homo sapiens
KWNKNWTATLGALTIRGHKLLCHLPHLLSSVQQTCRSSSR

SEQ ID NO: 316 - DNA, 120 bp, Homo sapiens
aagtggaacaagaactggacagccacactcggggctcttacaatcaggggtcataagctgctatgtcacctacctcaccttctcagctctgtccagcaaacctgcagaagtagttctaga

SEQ ID NO: 317 - protein, 50 aa, Homo sapiens
FKKFDGPCGERGGGRTARALWARGDSVLTPALDPQTPVRAPSLTRAAAAV

SEQ ID NO: 318 - DNA, 150 bp, Homo sapiens
ttcaagaagttcgacggcccttgtggtgagcgcggcggcgggcgcacggctcgagctctgtgggcgcgcggcgacagcgtcctgactcctgccctcgacccccagacccctgtcagggcgccctccctgacccgagccgcagctgccgtg

SEQ ID NO: 319 - protein, 52 aa, Homo sapiens
ENASLVFTGSNSPIPACELSSHPAHGISPWIPSPGNEHFHGIKKQVKAIKVE

SEQ ID NO: 320 - DNA, 156 bp, Homo sapiens
gaaaatgccagcctagtgtttacaggatccaacagccccataccagcctgcgaactgagtagtcacccagctcatggtatcagtccttggataccctcacctggaaatgaacatttccatggcataaagaagcaagtaaaggcaataaaagtagaa

SEQ ID NO: 321 - protein, 55 aa, Homo sapiens
RLTQRLVQGWTPMENRWCGRRAGGQPASSSTRWTTCRAACLLTKWTAGRSQTSIG

SEQ ID NO: 322 - DNA, 165 bp, Homo sapiens
cggctgacgcaacggctggtccagggctggaccccaatggagaacaggtggtgtggcaggcgagcgggtgggcagcccgcatcatccagcacgagatggaccacctgcagggctgcctgtttattgacaaaatggacagcaggacgttcacaaacgtctattgga

SEQ ID NO: 323 - protein, 32 aa, Homo sapiens
ENSGNASRWLHVPSSSDDWLGWKKSSAITSNS

SEQ ID NO: 324 - DNA, 96 bp, Homo sapiens
gaaaattcaggcaacgcctcgcgttggctgcatgtaccaagtagttcagacgattggctcggatggaaaaaatcttctgcaattacttccaattcc

SEQ ID NO: 325 - protein, 59 aa, Homo sapiens
GMECTLGQVGAPSPRREEDGWRGGHSRFKADVPAPQGPCWGGQPGSAPSSAPPEQSLLD

SEQ ID NO: 326 - DNA, 177 bp, Homo sapiens
ggcatggagtgcaccctggggcaggtgggtgccccgtcccctcggagggaggaggacggttggcgtgggggccacagccgattcaaggctgatgtaccagcaccgcagggaccctgctggggtggccaacctggctctgccccctcctcagctcctcctgaacagtcattattagat

SEQ ID NO: 327 - protein, 110 aa, Homo sapiens
KGSVERRSVSLGHPAEGWAWAERSLQPGMTTANTGCLSFHHRGCLLPVLPKLHCGLGGLPLVRAKEIKRVQRAGESSLPVKGLLTVASAVIAVLWGRPSEVTGENEAQHD

SEQ ID NO: 328 - DNA, 330 bp, Homo sapiens
aaagggagtgtggagaggcgctcggtgagcctggggcatcctgctgagggttgggcatgggcagagaggagcctccagccaggcatgaccacagccaacacaggctgcctctcattccaccacagagggtgcctcctccctgttttgcccaaattacactgtgggctaggtggactacctcttgtcagagctaaagaaatcaagcgagtgcagagggcaggggagagttcgctgcctgtgaagggccttctcaccgtcgcttcggctgtcatcgcagtcctgtggggtaggccaagcgaggtcacaggagaaaatgaggctcagcatgat

SEQ ID NO: 329 - protein, 75 aa, Homo sapiens
FGLTTLAGRSSHGTSGLRAATHTKSGDGGQGAARQCEKLLELARATRPWGRSTSASSRWTHRGYMCPPRCAVACW

SEQ ID NO: 330 - DNA, 225 bp, Homo sapiens
tttggcctcaccacacttgcaggtagaagctcccacgggacctcaggactgagggcagccacacacaccaagtctggggacggtggccagggggctgccaggcagtgtgagaagctcctggagctggcccgggctaccagaccctgggggaggagtacgtcagcatcatccaggtggacccatcgcggatacatgtgccctcctcgctgcgccgtggcgtgctgg

SEQ ID NO: 331 - protein, 29 aa, Homo sapiens
IIDSDKIMAVCMGCLLTRHVQCQAMEMQQ

SEQ ID NO: 332 - DNA, 87 bp, Homo sapiens
attattgacagcgacaagataatggcagtgtgcatggggtgcctgctcacacgtcatgtgcaatgccaggccatggagatgcaacag

SEQ ID NO: 333 - protein, 49 aa, Homo sapiens
DGHSYTSKVNCLLLQDGFHGCVSITGAAGRRNLSIFLFLMLCKLEFHAC

SEQ ID NO: 334 - DNA, 147 bp, Homo sapiens
gatggccactcctacacatccaaggtgaattgtttactccttcaagatgggttccatggctgtgtgagcatcaccggggcagctggaagaagaaacctgagcatcttcctgttcttgatgctgtgcaaattggagttccatgcttgt

SEQ ID NO: 335 - protein, 70 aa, Homo sapiens
LLNAEDYRCAIHSKEIYLLSPSPHQAMDKFSLCCINCNLCLHVFLLLLFFQNKDVWLISNIILLWIYGGI

SEQ ID NO: 336 - DNA, 210 bp, Homo sapiens
ctactaaatgcagaagattaccggtgtgccattcattcaaaagagatttatcttctttccccctccccccaccaggcaatggacaagttttctctctgctgcatcaactgcaatctatgtttacatgtattccttttactactattttttcaaaacaaagatgtatggcttatttcaaacatcattttactttggatatatggcggtatt

SEQ ID NO: 337 - protein, 33 aa, Homo sapiens
TGGKSTCSAPGPQSLPSTPFSTYPQWVILITEL

SEQ ID NO: 338 - DNA, 99 bp, Homo sapiens
acagggggcaaaagcacctgctcggctcctggccctcagtctctcccctccactccattctccacctacccacagtgggtcattctgatcaccgaactg

SEQ ID NO: 339 - protein, 28 aa, Homo sapiens
VETLENANSFSSGIQPLLCSLIGLENPT

SEQ ID NO: 340 - DNA, 84 bp, Homo sapiens
gtggagaccctggagaatgccaacagcttctccagcgggatccagccactcctctgttccctgattggcctggagaatcccacc

SEQ ID NO: 341 - protein, 38 aa, Homo sapiens
AGAGTISEGSVLHGQRLECDARRFFGCGTTILAEWEHH

SEQ ID NO: 342 - DNA, 114 bp, Homo sapiens
gctggggctggaacaatttccgaaggtagtgttctccatggtcagaggctggagtgtgatgccagacgtttttttgggtgtgggactacaatactggcagagtgggagcaccat

SEQ ID NO: 343 - protein, 17 aa, Homo sapiens
GVPGDSTRRAVRRMNTF

SEQ ID NO: 344 - DNA, 51 bp, Homo sapiens
ggagttccaggagattcaaccaggagagcagtgaggagaatgaataccttc

SEQ ID NO: 345 - protein, 20 aa, Homo sapiens
HVVGYGHLDTSGSSSSSSWP

SEQ ID NO: 346 - DNA, 60 bp, Homo sapiens
cacgtggtgggctatggccaccttgatacttccgggtcatcctcctcctcctcctggccc

SEQ ID NO: 347 - protein, 64 aa, Homo sapiens
WTPIPVLTRWPLPHPPPWRRATSCRMARSSPSATSGSSVRRRCSSLPSWVWNLAASTRPRSTPS

SEQ ID NO: 348 - DNA, 196 bp, Homo sapiens
tggacgcccatcccggtgctcacgagatggccactaccacatcctcctccctggagaagagctacaagctgccggatggccaggtcatcaccatcagcaacaagcggttccagtgtccggaggcgctgttccagccttccttcctgggtatggaatcttgcggcatccacgagaccacgttcaactccatcatgaa

SEQ ID NO: 349 - protein, 16 aa, Homo sapiens
SLYHREKQLIAMDSAI

SEQ ID NO: 350 - DNA, 48 bp, Homo sapiens
tccctctaccaccgggagaagcagctcattgctatggacagtgctatc

SEQ ID NO: 351 - protein, 43 aa, Homo sapiens
LHPQRETFTPRWSGANYWKLAFPVGAEGTFPAAATQRGVVRPA

SEQ ID NO: 352 - DNA, 131 bp, Homo sapiens
cttcatcctcagagggaaacattcactccccggtggtcgggcgcgaattactggaaattggcttttcccgttggggccgaaggtaccttccctgcggcggcgactcagcggggtgtcgttcggccggcgtg

SEQ ID NO: 353 - protein, 35 aa, Homo sapiens
NSKMALNSLNSIDDAQLTRIAPPRSHCCFWEVNAP

SEQ ID NO: 354 - DNA, 105 bp, Homo sapiens
aacagcaagatggctttgaactcattaaactccattgatgatgcacagttgacaagaattgcccctccaagatctcattgctgtttctgggaagtaaacgctcct

SEQ ID NO: 355 - protein, 50 aa, Homo sapiens
MAGGVLRRLLCREPDRDGDKGASREETVVPLHIGDPVVLPGIGQCYSALF

SEQ ID NO: 356 - DNA, 151 bp, Homo sapiens
atggctggaggagtccttcggcggctgttgtgtcgggagcctgatcgcgatggggacaaaggcgcaagtcgagaggaaactgttgtgcctcttcatattggcgatcctgttgtgctccctggcattgggcagtgttacagtgcactcttct

SEQ ID NO: 357 - protein, 39 aa, Homo sapiens
DDRNTFDIVWWCPMSRLRLALTVPPSTTTTCVTVPAWAA

SEQ ID NO: 358 - DNA, 120 bp, Homo sapiens
gatgacagaaacactttcgacatagtgtggtggtgccctatgagccgcctgaggttggctctgactgtaccaccatccactacaactacatgtgtaacagttcctgcatgggcggcatga

SEQ ID NO: 359 - protein, 17 aa, Homo sapiens
VQPIARELYQFTFDLLI

SEQ ID NO: 360 - DNA, 51 bp, Homo sapiens
gtgcagcctattgcgagagagctgcatcagttcacttttgacctgctaatc

SEQ ID NO: 361 - protein, 17 aa, Homo sapiens
QMAVIQYSCMGLMVFAM

SEQ ID NO: 362 - DNA, 51 bp, Homo sapiens
cagatggctgtcattcagtactcctgcatggggctcatggtgtttgccatg

SEQ ID NO: 363 - protein, 17 aa, Homo sapiens
PKSEVRAKCKFSILNAK

SEQ ID NO: 364 - DNA, 50 bp, Homo sapiens
caaagagtgaagttcgggcaaaattcaaatgctccatcctgaatgccaag

SEQ ID NO: 365 - protein, 17 aa, Homo sapiens
MMAEIISVHVPKILSGK

SEQ ID NO: 366 - DNA, 51 bp, Homo sapiens
atgatggcagagatcatctctgtgcacgtgcccaagatcctttctgggaaa

SEQ ID NO: 367 - protein, 17 aa, Homo sapiens
LHPDSGNMVENGCYLRR

SEQ ID NO: 368 - DNA, 51 bp, Homo sapiens
ctgcacccggactccggcaacatggtcgagaacggctgctacttgcgccgc

SEQ ID NO: 369 - protein, 17 aa, Homo sapiens
CYLRRQKRLKCEKQPGA

SEQ ID NO: 370 - DNA, 51 bp, Homo sapiens
tgctacttgcgccgccagaagcgcttgaagtgcgagaagcagccgggggcc

SEQ ID NO: 371 - protein, 17 aa, Homo sapiens
MFENGCYLGRQKRFKCE

SEQ ID NO: 372 - DNA, 51 bp, Homo sapiens
atgttcgagaacggctgctacttgggccgccagaagcgcttcaagtgcgag

SEQ ID NO: 373 - protein, 16 aa, Homo sapiens
DSSGNLLERNSFEVRV

SEQ ID NO: 374 - DNA, 48 bp, Homo sapiens
gactccagtggtaatctactggaacggaacagctttgaggtgcgtgtt

SEQ ID NO: 375 - protein, 61 aa, Homo sapiens
VFFKRAAEGFFRMNKLKESSDTNPKPYCMAAPMGLTENNRNRKKSYRETNLKAVSWPLNHT

SEQ ID NO: 376 - DNA, 183 bp, Homo sapiens
gtcttcttcaaaagagccgctgaaggatttttcagaatgaacaaattaaaagaatcatcagacactaaccccaagccatactgcatggcagcaccaatgggactgacagaaaacaacagaaataggaagaaatcctacagagaaacaaacttgaaagctgtctcatggcctttgaatcatact

SEQ ID NO: 377 - protein, 15 aa, Homo sapiens
LTFLDFIQVTLRVMS

SEQ ID NO: 378 - protein, 15 aa, Homo sapiens
VTLRVMSGSQMENGS

SEQ ID NO: 379 - protein, 24 aa, Homo sapiens
YEAGMTLGEKFRVGNCKHLKMTRP

SEQ ID NO: 380 - DNA, 72 bp, Homo sapiens
tatgaagcagggatgactctgggagaaaaattccgggttggcaattgcaagcatctcaaaatgaccagaccc

SEQ ID NO: 381 - protein, 28 aa, Homo sapiens
YEAGMTLGGKILFFLFLLLPLSPFSLIF

SEQ ID NO: 382 - DNA, 84 bp, Homo sapiens
tatgaagcagggatgactctgggaggtaagatacttttctttctcttcctcctccttcctctctcccccttctccctcattttc

SEQ ID NO: 383 - protein, 16 aa, Homo sapiens
GYLRMQGLMAQRLLLR

SEQ ID NO: 384 - DNA, 48 bp, Homo sapiens
gggtacctgaggatgcagggactcatggctcagcgacttcttctgagg

SEQ ID NO: 385 - protein, 48 aa, Homo sapiens
DGHSYTSKVNCLLLQDGFHGCVSITGAAGRRNLSIFLFLMLCKLEFHA

SEQ ID NO: 386 - DNA, 144 bp, Homo sapiens
gatggccactcctacacatccaaggtgaattgtttactccttcaagatgggttccatggctgtgtgagcatcaccggggcagctggaagaagaaacctgagcatcttcctgttcttgatgctgtgcaaattggagttccatgct

SEQ ID NO: 387 - protein, 9 aa, Homo sapiens
STRRAVRRM

SEQ ID NO: 388 - protein, 9 aa, Homo sapiens
RAVRRMNTF

SEQ ID NO: 389 - protein, 9 aa, Homo sapiens
IPVLTRWPL

SEQ ID NO: 390 - protein, 9 aa, Homo sapiens
QLIAMDSAI

SEQ ID NO: 391 - protein, 9 aa, Homo sapiens
FPVGAEGTF

SEQ ID NO: 392 - protein, 9 aa, Homo sapiens
NSKMALNSL

SEQ ID NO: 393 - protein, 9 aa, Homo sapiens
GVLRRLLCR

SEQ ID NO: 394 - protein, 9 aa, Homo sapiens
CPMSRLRLA

SEQ ID NO: 395 - protein, 9 aa, Homo sapiens
YQFTFDLLI

SEQ ID NO: 396 - protein, 9 aa, Homo sapiens
IQYSCMGLM

SEQ ID NO: 397 - protein, 9 aa, Homo sapiens
RAKCKFSIL

SEQ ID NO: 398 - protein, 9 aa, Homo sapiens
HVPKILSGK

SEQ ID NO: 399 - protein, 9 aa, Homo sapiens
NMVENGCYL

SEQ ID NO: 400 - protein, 9 aa, Homo sapiens
YLRRQKRLK

SEQ ID NO: 401 - protein, 9 aa, Homo sapiens
CYLGRQKRF

SEQ ID NO: 402 - protein, 9 aa, Homo sapiens
LLERNSFEV

SEQ ID NO: 403 - protein, 9 aa, Homo sapiens
YCMAAPMGL

SEQ ID NO: 404 - protein, 9 aa, Homo sapiens
FFKRAAEGF

SEQ ID NO: 405 - protein, 9 aa, Homo sapiens
RVGNCKHLK

SEQ ID NO: 406 - protein, 9 aa, Homo sapiens
FLFLLLPLS

SEQ ID NO: 407 - protein, 9 aa, Homo sapiens
LMAQRLLLR

SEQ ID NO: 408 - protein, 9 aa, Homo sapiens
IVWWCPMSR

SEQ ID NO: 409 - protein, 9 aa, Homo sapiens
CTELKLSDY

SEQ ID NO: 410 - protein, 9 aa, Homo sapiens
NLVPMVATV

SEQ ID NO: 411 - protein, 9 aa, Homo sapiens
LIYRRRLMK

SEQ ID NO: 412 - protein, 9 aa, Homo sapiens
LYSACFWWL

SEQ ID NO: 413 - protein, 9 aa, Homo sapiens
NPKASLLSL

SEQ ID NO: 414 - protein, 9 aa, Homo sapiens
ELRSRYWAI

SEQ ID NO: 415 - protein, 15 aa, Homo sapiens
SQMENGSSYFFKPFS

SEQ ID NO: 416 - protein, 9 aa, Homo sapiens
IVTDFSVIK

SEQ ID NO: 417 - protein, 15 aa, Homo sapiens
YFFKPFSWGLGVGLS

SEQ ID NO: 418 - protein, 15 aa, Homo sapiens
FMIGELVGELCCQLT

SEQ ID NO: 419 - protein, 15 aa, Homo sapiens
ELCCQLTFRLPFLES

SEQ ID NO: 420 - protein, 15 aa, Homo sapiens
RLPFLESLCQAVVTQ

SEQ ID NO: 421 - protein, 9 aa, Homo sapiens
KLSRALRYY

SEQ ID NO: 422 - protein, 9 aa, Homo sapiens
MVFELANSI

SEQ ID NO: 423 - protein, 9 aa, Homo sapiens
ILFQNIDGK

SEQ ID NO: 424 - protein, 9 aa, Homo sapiens
KIVIARYGK

SEQ ID NO: 425 - protein, 9 aa, Homo sapiens
FLLELLSDS

SEQ ID NO: 426 - protein, 10 aa, Homo sapiens
VTFLKPCFLL

SEQ ID NO: 427 - protein, 8 aa, Homo sapiens
TDIVKQSV

SEQ ID NO: 428 - protein, 10 aa, Homo sapiens
SPAFPKPVRP

SEQ ID NO: 429 - protein, 12 aa, Homo sapiens
SYFSLTNIFNFV

SEQ ID NO: 430 - protein, 12 aa, Homo sapiens
EFSPETCAFRLS

SEQ ID NO: 431 - protein, 9 aa, Homo sapiens
FLSRALRAL

SEQ ID NO: 432 - protein, 8 aa, Homo sapiens
KKDLELIL

SEQ ID NO: 433 - protein, 8 aa, Homo sapiens
KLQKNCLL

SEQ ID NO: 434 - protein, 9 aa, Homo sapiens
SALSGNSWV

SEQ ID NO: 435 - protein, 7 aa, Homo sapiens
TVRAILL

SEQ ID NO: 436 - protein, 10 aa, Homo sapiens
GSLHFHEVLK

SEQ ID NO: 437 - protein, 75 aa, Homo sapiens
HYKLIQQPISLFSITDRLHKTFSQLPSVHLCSITFQWGHPPIFCSTNDICVTANFCISVTFLKPCFLLHEASASQ

SEQ ID NO: 438 - protein, 40 aa, Homo sapiens
WTDIVKQSVSTNCISIKKGSYTKLFSLVFLIFCWPLIIQL

SEQ ID NO: 439 - protein, 144 aa, Homo sapiens
RTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLG

SEQ ID NO: 440 - protein, 36 aa, Homo sapiens
LRYGALCNVSRISYFSLTNIFNFVIKSLTAIFTVKF

SEQ ID NO: 441 - protein, 295 aa, Homo sapiens
RKERNIRKSESTLRLSPFPTPAPSGAPAAAQGKVVRVPGPAGGLVPRDAGARLLPPAGGPGGGAAAGEGRAGRGRFPSITEPRPRDLPPRVATGRRAGGRRKGAGQGVRTRPLPASWPGGRGPFRKGPRRLPLGSGPPAAGVQRLRCSHLSRGPRRRRGRVCGRACVSPPLPPRPPPVGLSAENLSWLSSGLPRACSWREFSPETCAFRLSGLDSKLSARVERDLGALRAPGSRAAQGGGRVRGSRSEWKTRPWRPPPAWPLTRAGGPLPKNPFLESCSETAQRRRVFSFSTPLS

SEQ ID NO: 442 - protein, 67 aa, Homo sapiens
YAYKDFLWCFPFSLVFLQEIQICCHVSCLCCICCSTRICLGCLLELFLSRALRALHVLWNGFQLHCQ

SEQ ID NO: 443 - protein, 82 aa, Homo sapiens
SINKATITGKKDLELILHVSRKKPFLPRVNITPTPISCCNLKMLKKFFLLYIIISIIDLTNCLSCYLEHFYRFTFFTDVHYF

SEQ ID NO: 444 - protein, 16 aa, Homo sapiens
TMPAILKLQKNCLLSL

SEQ ID NO: 445 - protein, 84 aa, Homo sapiens
PYYSALSGNSWVPSTLESDPFGYVFSPLATRPALNDQESILWPTLTSVVSCALSCPSLNLPENWLTLITGGMKGGKKMKFTFRH

SEQ ID NO: 446 - protein, 46 aa, Homo sapiens
GLRNLGNTVRAILLSFLSKRNVKWCWGWGKPTSLGKACGRRALKLF

SEQ ID NO: 447 - protein, 22 aa, Homo sapiens
MEAENAGSLHFHEVLKMGHVKF

SEQ ID NO: 448 - DNA, 225 bp, Homo sapiens
cactacaaattaattcaacaacccatatccctcttctccatcactgataggctccataagacgttcagtcagctgccctcggtccatctctgctcaatcaccttccagtggggacacccgcccattttctgctcaacaaatgatatctgtgtcacggccaacttctgcatctcggtcacattccttaaaccgtgcttcctcctacatgaggcatctgcctcacag

SEQ ID NO: 449 - DNA, 120 bp, Homo sapiens
tggactgatatagttaagcagtctgtaagtacaaactgcatttctatcaagaaaggtagctatacaaaactgttttccttagtctttcttattttctgttggccattaattattcagttg

SEQ ID NO: 450 - DNA, 432 bp, Homo sapiens
aggaccgccctgacacacaatcaggacttctctatctacaggctctgttgcaagagggggtccctctgccacgcttcccaggccagatccccggctttcccgaagccggtcagacctcttcctgcccccatcaccagaatcaccccccaactggggggacaatctgactcgagtcaaccccttctcactacgggaagacctcaggggtggcaagatcaagctcttagacacacccagcaagccagtcctgcctcttgtgccaccatcaccattcccatccactcagctgcccttggtgaccactccggagaccctggtccagcctgggacacctgcccgccgctgccgctcactaccctcatcccccgagctcccccgccgtatggagacagcactgccaggtcctggccctcccgctgtgggcccctcggc

SEQ ID NO: 451 - DNA, 108 bp, Homo sapiens
cttcgctatggtgccttatgcaatgtaagtagaataagttatttcagtctaacaaatatatttaattttgtaattaaatcactaactgctatttttactgtgaaattt

SEQ ID NO: 452 - DNA, 885 bp, Homo sapiens
cgaaaagagagaaacatccgaaaaagtgagtccacgctgcgcctgtccccgttccccacccccgccccgtcgggggcgcccgcggccgcgcaggggaaagttgtccgggtccccgggccggcgggcgggctggtcccccgggacgctggcgctcggctcctgcccccggcgggcggcccggggggaggggcggcggcgggggaggggcgcgcgggccgcggccggttccctagcatcacggagcctcgaccccgcgacctcccgccccgggtcgccaccggccggcgggcgggaggccggcggaaaggcgccgggcagggcgtgcgcacccgtcccttgcccgcgagctggcccgggggtcgcggccctttccggaaggggccccggcgtctgccgctgggctccggcccgcccgctgcgggagtgcagcggctgcgttgctcccacctgagccgcgggccgaggaggcggaggggccgagtgtgcgggagggcgtgtgtctcgcctccccttcctccccggcccccgcctgtcggcctttctgctgagaacctaagctggttgtcaagtggtttgcctcgggcgtgttcctggcgcgagttcagccccgagacctgtgcgtttcggctctcgggtttggattcgaaactttccgctcgggttgagcgtgacttgggtgcgctgcgggcgccggggtcgcgggctgcgcagggcggtgggcgtgtgcgcgggagccggtcggagtggaaaacgcgcccgtggcggccacctccagcctggccgctcacccgagcaggggggccgctgcccaagaaccctttcctggagagctgctccgagaccgcacagcgccgccgcgtcttctccttttccactcctctctcc

SEQ ID NO: 453 - DNA, 201 bp, Homo sapiens
tatgcttacaaggactttctctggtgttttcctttttctttagtttttctccaagagattcaaatctgctgccatgttagctgtctttgctgtatctgctgtagtacacgaatatgccttggctgtttgcttgagctttttctatcccgtgctcttcgtgctcttcatgttctttggaatggctttcaacttcattgtcaa

SEQ ID NO: 454 - DNA, 246 bp, Homo sapiens
tccatcaacaaagccaccataacaggtaagaaagatctggagcttattcttcatgtgtctaggaagaaaccatttctgccaagagtcaatataacaccaacaccaatttcatgctgcaatttgaaaatgttaaagaaattctttcttctctacattatcatttctatcattgatctcacaaattgtctaagctgttatttggaacatttttaccgatttacgttttttactgatgtacattatttt

SEQ ID NO: 455 - DNA, 48 bp, Homo sapiens
accatgcctgctattttaaagttacagaagaattgtcttctctcctta

SEQ ID NO: 456 - DNA, 252 bp, Homo sapiens
ccatactacagcgcactgtcaggtaacagctgggttcccagcaccctggaaagtgacccgtttggctatgtttttagccccttagcaacacggccagctctcaatgaccaagagtccatcttgtggccgaccctgacttctgtggtttcctgtgctctatcctgcccatctcttaacttacctgagaattggctcactctcatcacaggtggaatgaaagggggaaaaaaaatgaaattcacattcagacac

SEQ ID NO: 457 - DNA, 138 bp, Homo sapiens
ggccttcgaaacctgggaaacacggtgagagctattctcctatctttcctctctaaaaggaatgtgaaatggtgctgggggtggggaaaacccacgagcttggggaaggcatgtggaaggagagctctgaagctcttc

SEQ ID NO: 458 - DNA, 66 bp, Homo sapiens
atggaagcggagaacgcgggcagtttgcattttcatgaagtgctcaaaatgggacatgtgaaattc

SEQ ID NO: 459 - DNA, 81 bp, Artificial sequence
  Codon-optimized polynucleotide
tggaagttcgagatgagctacaccgtcggcggacctccacctcatgttcatgccagacctcggcactggaaaaccgacaga

SEQ ID NO: 460 - DNA, 84 bp, Artificial sequence
  Codon-optimized polynucleotide
tatgaggccggcatgacactcggcggcaagatcctgttcttcctgttcctgctgctccctctgagccccttcagcctgatcttc

SEQ ID NO: 461 - DNA, 147 bp, Artificial sequence
  Codon-optimized polynucleotide
gatggccacagctacaccagcaaagtgaactgcctcctgctgcaggatggcttccacggctgtgtgtctattactggcgccgctggcagacggaacctgagcatctttctgtttctgatgctgtgcaagctcgagttccacgcctgc

SEQ ID NO: 462 - DNA, 99 bp, Artificial sequence
  Codon-optimized polynucleotide
acaggcggcaagtccacatgttctgcccctggacctcagagcctgcctagcacacccttcagcacataccctcagtgggtcatcctgatcaccgaactc

SEQ ID NO: 463 - DNA, 42 bp, Artificial sequence
  Codon-optimized polynucleotide
gtgctgagattcctggacctgaaagtgcgctacctgcacagc

SEQ ID NO: 464 - DNA, 54 bp, Artificial sequence
  Codon-optimized polynucleotide
gactattgggctcaaaaagagaagggcagcagcagcttcctgcggcctagctgt

SEQ ID NO: 465 - DNA, 183 bp, Artificial sequence
  Codon-optimized polynucleotide
gtccccttcagagagctgaagaacgtttccgtgctggaaggcctgagacagggcagacttggcggcccttgtagctgtcactgccccagacctagtcaggccagactgacacctgtggatgtggccggacctttcctgtgtctgggagatcctggcctgtttccacctgtgaagtccagcatc

SEQ ID NO: 466 - DNA, 177 bp, Artificial sequence
  Codon-optimized polynucleotide
ggcatggaatgcaccctgggacaagtgggagccccatctcctagaagagaagaggatggctggcgcggaggccactctagattcaaagctgatgtgcccgctcctcagggcccttgttggggaggacaacctggatctgccccatcttctgccccacctgaacagtccctgctggat

SEQ ID NO: 467 - DNA, 96 bp, Artificial sequence
  Codon-optimized polynucleotide
ggcaacacaaccctccagcaactgggagaagcctctcaggctcctagcggctctctgatccctctcagactgcctctcctgtgggaagttcggggc

SEQ ID NO: 468 - DNA, 129 bp, Artificial sequence
  Codon-optimized polynucleotide
gaggctttccaaagagctgctggcgaaggcggacctggtagaggtggtgctagaagaggtgctagggtgctgcagagcccattctgtagagcaggcgcaggcgaatggctgggccatcagagtctgaga

SEQ ID NO: 469 - DNA, 51 bp, Artificial sequence
  Codon-optimized polynucleotide
gattattgggcccagaaagaaaagatcagcatccccagaacacacctgtgc

SEQ ID NO: 470 - DNA, 48 bp, Artificial sequence
  Codon-optimized polynucleotide
gtggccatgatggtgcccgatagacaggtccactacgactttggactg

SEQ ID NO: 471 - DNA, 270 bp, Artificial sequence
  Codon-optimized polynucleotide
gtgcccttccgggaactgaagaaccagagaacagctcagggcgctcctggaatccaccatgctgcttctccagtggccgccaacctgtgtgatcctgccagacatgcccagcacaccaggattccttgtggcgctggacaagtgcgcgctggaagaggacctgaagcaggcggaggtgttctgcaacctcaaagacccgctcctgagaagcctggctgcccttgcagaagaggacagcctagactgcacaccgtgaaaatgtggcgagcc

SEQ ID NO: 472 - DNA, 33 bp, Artificial sequence
  Codon-optimized polynucleotide
aagagaagctttgccgtgaccgagcggatcatc

SEQ ID NO: 473 - DNA, 150 bp, Artificial sequence
  Codon-optimized polynucleotide
ttcaagaagttcgacggcccttgcggagaaagaggcggaggcagaacagctagagccctttgggctagaggcgacagcgttctgacaccagctctggaccctcagacacctgttagggcccctagcctgacaagagctgccgccgctgtg

SEQ ID NO: 474 - DNA, 42 bp, Artificial sequence
  Codon-optimized polynucleotide
ctggtgctgggagtgctgtctggacactctggcagcagactg

SEQ ID NO: 475 - DNA, 66 bp, Artificial sequence
  Codon-optimized polynucleotide
cagtggcagcactatcacagatctggcgaagccgccggaacacccctttggaggccaacaagaaac

SEQ ID NO: 476 - DNA, 285 bp, Artificial sequence
  Codon-optimized polynucleotide
tgccacttgtttctgcagccccaagtgggcacacctcctccacatacagcctctgctagagcacctagcggccctccacatcctcacgaatcttgtcctgccggaagaaggcctgccagagccgctcaaacatgtgccagacgacagcacggactgcctggatgtgaagaggctggaacagccagagtgcctagcctgcacctccatctgcatcaggctgctcttggagccggaagaggtagaggatggggcgaagcttgtgctcaggtgccaccttctagaggc

SEQ ID NO: 477 - DNA, 27 bp, Artificial sequence
  Codon-optimized polynucleotide
aagatccagaacaagaactgccccgac

SEQ ID NO: 478 - DNA, 225 bp, Artificial sequence
  Codon-optimized polynucleotide
cactacaagctgatccagcagccaatcagcctgttcagcatcaccgaccggctgcacaagacattcagccagctgccaagcgtgcacctgtgctccatcaccttccagtggggacaccctcctatcttttgctccaccaacgacatctgcgtgaccgccaacttctgtatcagcgtgaccttcctgaagccttgctttctgctgcacgaggccagcgcctctcag

SEQ ID NO: 479 - DNA, 432 bp, Artificial sequence
  Codon-optimized polynucleotide
cgaaccgctctgacacacaaccaggacttcagcatctacagactgtgttgcaagcggggctccctgtgccatgcaagccaagctagaagccccgcctttcctaaacctgtgcgacctctgccagctccaatcaccagaattacccctcagctcggcggccagagcgattcatctcaacctctgctgaccaccggcagacctcaaggctggcaagaccaagctctgagacacacccagcaggctagccctgcctcttgtgccaccatcacaatccccatccactctgccgctctgggcgatcattctggcgatcctggaccagcctgggacacatgtcctccactgccactcacaacactgatccctagggctcctccaccttacggcgattctaccgctagaagctggcccagcagatgtggaccactcgga

SEQ ID NO: 480 - DNA, 201 bp, Artificial sequence
  Codon-optimized polynucleotide
tacgcctacaaggacttcctgtggtgcttccccttctctctggtgttcctgcaagagatccagatctgctgtcatgtgtcctgcctgtgctgcatctgctgtagcaccagaatctgcctgggctgtctgctggaactgttcctgagcagagccctgagagcactgcacgtgctgtggaacggattccagctgcactgccag

SEQ ID NO: 481 - DNA, 48 bp, Artificial sequence
  Codon-optimized polynucleotide
acaatgcccgccatcctgaagctgcagaagaattgcctcctaagcctg

SEQ ID NO: 482 - DNA, 72 bp, Artificial sequence
  Codon-optimized polynucleotide
tacgaagccgggatgaccctgggcgagaagttcagagtgggcaactgcaagcacctgaagatgacccggcct

SEQ ID NO: 483 - DNA, 51 bp, Artificial sequence
  Codon-optimized polynucleotide
ggcgtgccaggcgatagcactcggagagccgtcagacggatgaacaccttt

SEQ ID NO: 484 - DNA, 48 bp, Artificial sequence
  Codon-optimized polynucleotide
tgtggcgcctctgcctgtgacgtgtccctgatcgctatggactccgcc

SEQ ID NO: 485 - DNA, 48 bp, Artificial sequence
  Codon-optimized polynucleotide
agcctgtaccaccgggaaaagcagctcattgccatggacagcgccatc

SEQ ID NO: 486 - DNA, 48 bp, Artificial sequence
  Codon-optimized polynucleotide
accgagtacaaccagaaactgcaagtgaaccagttcagcgagagcaag

SEQ ID NO: 487 - DNA, 72 bp, Artificial sequence
  Codon-optimized polynucleotide
accgagatcagctgctgcaccctgagcagcgaggaaaacgagtacctgcctagacctgagtggcagctgcag

SEQ ID NO: 488 - DNA, 42 bp, Artificial sequence
  Codon-optimized polynucleotide
tgcgaagagagaggcgccgcaggatctctgatctcctgcgaa

SEQ ID NO: 489 - DNA, 48 bp, Artificial sequence
  Codon-optimized polynucleotide
aacagcaagatggccctgaatagcgaggccctgtctgtggtgtctgaa

SEQ ID NO: 490 - DNA, 210 bp, Artificial sequence
  Codon-optimized polynucleotide
tggggcatggaactggccgccagcagaagattcagctgggatcatcatagcgcaggcggcccacctagagtgccatctgttagaagcggagctgcccaggtgcagcctaaagatcctctgccactgagaacactggccggctgccttgctagaacagcccatcttagacctggcgccgagtctctgcctcagccacaactgcactgtacc

SEQ ID NO: 491 - DNA, 60 bp, Artificial sequence
  Codon-optimized polynucleotide
catgtcgtcggctacggccacctggatacaagcggaagcagctctagctccagctggcct

SEQ ID NO: 492 - DNA, 105 bp, Artificial sequence
  Codon-optimized polynucleotide
aactcaaaaatggctctgaacagcctgaactccatcgacgacgcccagctgacaagaatcgcccctcctagatctcactgctgcttttgggaagtgaacgcccca

SEQ ID NO: 493 - DNA, 48 bp, Artificial sequence
  Codon-optimized polynucleotide
aagatgcactttagcctgaaagaacaccctccaccaccttgtcctcca

SEQ ID NO: 494 - DNA, 207 bp, Artificial sequence
  Codon-optimized polynucleotide
ctgtggttccagtccagcgagctgtctcctactggtgccccttggccatctagacgccctacttggagaggcaccaccgtgtcaccaagaaccgccacaagcagcgccagaacctgttgtggcacaaagtggccctccagccaagaagccgctctcggacttggaagcggactgctgaggttctcttgtggaaccgccgccattcgg

SEQ ID NO: 495 - DNA, 51 bp, Artificial sequence
  Codon-optimized polynucleotide
atcgctagagagctgcaccagttcgccttcgacctgctgatcaagagccac

SEQ ID NO: 496 - DNA, 51 bp, Artificial sequence
  Codon-optimized polynucleotide
cagcctgattcttttgccgcactgcacagctccctgaacgagctgggagag

SEQ ID NO: 497 - DNA, 51 bp, Artificial sequence
  Codon-optimized polynucleotide
ttcgtgcaaggcaaggattggggcctcaaaaagtttatccgcagagacttc

SEQ ID NO: 498 - DNA, 51 bp, Artificial sequence
  Codon-optimized polynucleotide
tttgtgcagggcaaagactggggcgtgaagaagttcatccggcgggacttc

SEQ ID NO: 499 - DNA, 204 bp, Artificial sequence
  Codon-optimized polynucleotide
cagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgccttcacc

SEQ ID NO: 500 - DNA, 81 bp, Artificial sequence
  Codon-optimized polynucleotide
tggaagttcgagatgagctacaccgttggcggccctccaccacatgttcacgccagacctagacactggaaaaccgacaga

SEQ ID NO: 501 - DNA, 84 bp, Artificial sequence
  Codon-optimized polynucleotide
tacgaggccggcatgacactcggaggcaagatcctgttcttcctgttcctgctgctccctctgagccccttcagcctgatcttt

SEQ ID NO: 502 - protein, 15 aa, Homo sapiens
CQAVVTQALRFNPSF

SEQ ID NO: 503 - DNA, 99 bp, Artificial sequence
  Codon-optimized polynucleotide
acaggcggcaagagcacatgttctgcccctggacctcagtctctgcccagcacacccttcagcacataccctcagtgggtcatcctgatcaccgagctg

SEQ ID NO: 504 - DNA, 42 bp, Artificial sequence
  Codon-optimized polynucleotide
gtgctgcggttcctggatctcaaagtgcgctacctgcacagc

SEQ ID NO: 505 - DNA, 54 bp, Artificial sequence
  Codon-optimized polynucleotide
gattattgggcccagaaagaaaagggcagcagcagcttcctgcggcctagctgt

SEQ ID NO: 506 - DNA, 183 bp, Artificial sequence
  Codon-optimized polynucleotide
gtgcccttccgggaactgaagaacgtgtccgttctggaaggcctgaggcagggcagacttggcggaccttgtagctgccactgtcctagaccaagccaggccagactgacccctgtggatgtggctggcccatttctgtgtctgggcgaccctggactgttccctccagtgaagtctagcatc

SEQ ID NO: 507 - DNA, 177 bp, Artificial sequence
  Codon-optimized polynucleotide
ggcatggaatgtacactgggccaagtgggagccccatctcctagaagagaagaggatggctggcgcggaggccactctagattcaaagctgatgtgcccgctcctcagggcccttgttggggaggacaacctggatctgccccatcttctgccccacctgaacagagcctgctggat

SEQ ID NO: 508 - DNA, 96 bp, Artificial sequence
  Codon-optimized polynucleotide
ggcaacaccacactgcaacagctgggagaagcctctcaggccccaagcggttctctgatccctctcagactgcccctcctgtgggaagtgcggggc

SEQ ID NO: 509 - DNA, 129 bp, Artificial sequence
  Codon-optimized polynucleotide
gaggctttccagagagcagctggcgaaggcggacctggcagaggtggtgctagaagaggtgctagagtgctgcagagcccattctgtagagctggcgctggcgaatggctgggccaccaatctcttaga

SEQ ID NO: 510 - DNA, 51 bp, Artificial sequence
  Codon-optimized polynucleotide
gactattgggctcaaaaagagaagatcagcatccccagaacacacctgtgc

SEQ ID NO: 511 - DNA, 48 bp, Artificial sequence
  Codon-optimized polynucleotide
gtggccatgatggtgcccgacagacaggtgcactacgacttcggcctg

SEQ ID NO: 512 - DNA, 270 bp, Artificial sequence
  Codon-optimized polynucleotide
gtgcccttcagagagctgaaaaaccagagaacagcccagggcgctcctggaatccatcatgctgcttctccagtggccgccaatctgtgcgatcctgccagacatgcccagcataccaggattccttgtggcgctggacaagtgcgcgctggaagaggacctgaagctggtggcggagttctgcagcctcaaagacctgctcctgagaagcctggctgcccctgtagaagaggacagcctagactgcacaccgtgaagatgtggcgggcc

SEQ ID NO: 513 - DNA, 33 bp, Artificial sequence
  Codon-optimized polynucleotide
aagagaagcttcgccgtgaccgagcggatcatc

SEQ ID NO: 514 - DNA, 150 bp, Artificial sequence
  Codon-optimized polynucleotide
tttaagaagtttgacggcccctgcggcgagagaggcggaggaagaactgcaagagccctttgggccagaggcgactctgttctgacaccagctctggaccctcagacacctgttagggcccctagcctgacaagagctgccgctgctgtt

SEQ ID NO: 515 - DNA, 42 bp, Artificial sequence
  Codon-optimized polynucleotide
ctggtgctgggcgtgctgtctggccactctggaagcagactg

SEQ ID NO: 516 - DNA, 66 bp, Artificial sequence
  Codon-optimized polynucleotide
caatggcagcactaccacagatctggcgaagccgctggaaccccactttggaggcctaccagaaac

SEQ ID NO: 517 - DNA, 285 bp, Artificial sequence
  Codon-optimized polynucleotide
tgccacttgtttctccagccacaagtgggcacccctccacctcatacagcctctgctagagcacctagcggcccacctcatcctcacgaatcttgtcctgccggaagaaggcctgccagagccgctcaaacatgtgccagacgacagcacggactgcccggatgtgaagaagccggaacagccagagtgcctagcctgcaccttcatctgcatcaggccgctcttggagccggaagaggtagaggatggggagaagcttgtgcccaggtgccaccttctagaggc

SEQ ID NO: 518 - protein, 15 aa, Homo sapiens
LRFNPSFQEVCIYQD

SEQ ID NO: 519 - DNA, 225 bp, Artificial sequence
  Codon-optimized polynucleotide
cactacaagctgatccagcagccaatcagcctgttctccatcaccgaccggctgcacaagacattcagccagctgccttccgtgcatctgtgcagcatcaccttccagtggggacaccctcctatcttttgctccaccaacgacatctgcgtgaccgccaacttctgtatcagcgtgaccttcctgaagccttgctttctgctgcacgaggcctccgccagccag

SEQ ID NO: 520 - DNA, 432 bp, Artificial sequence
  Codon-optimized polynucleotide
cggaccgctctgacccacaaccaggacttcagcatctaccggctgtgctgcaagaggggctctctgtgtcatgctagccaggctagaagccccgcctttcctaagcctgtcagacctctgcctgctcctatcaccagaatcacccctcagctcggcggccagtctgattcatctcagccactgctgaccaccggcagacctcaaggatggcaagaccaggctctgagacacacacagcaggctagcccagcctcttgcgccaccatcacaataccaatacattctgccgctctgggcgatcacagcggagatcctggacctgcctgggatacttgtcctcctctgcccctaactacactgatccctagggctcctccaccttacggcgatagcacagccagatcctggcctagcagatgtggccctctgggc

SEQ ID NO: 521 - DNA, 201 bp, Artificial sequence
  Codon-optimized polynucleotide
tacgcctacaaggacttcctgtggtgcttccccttctctctggtgttcctgcaagaaatccagatctgctgtcacgtgtcctgcctgtgctgtatctgctgtagcacccggatctgtctgggctgtctgctggaactgttcctgagcagagccctgagagcactgcacgtgctgtggaacggattccagctgcactgccag

SEQ ID NO: 522 - DNA, 48 bp, Artificial sequence
  Codon-optimized polynucleotide
accatgcctgccattctgaagctgcagaagaattgtcttctaagcctg

SEQ ID NO: 523 - DNA, 72 bp, Artificial sequence
  Codon-optimized polynucleotide
tatgaggctggaatgaccctgggcgagaagttcagagtgggcaactgcaagcacctgaagatgacccggcct

SEQ ID NO: 524 - DNA, 51 bp, Artificial sequence
  Codon-optimized polynucleotide
ggagtgcctggcgattctactagaagggccgtgcggcggatgaacaccttt

SEQ ID NO: 525 - DNA, 48 bp, Artificial sequence
  Codon-optimized polynucleotide
tgtggcgcatctgcctgcgacgtgtccctgatcgctatggatagcgcc

SEQ ID NO: 526 - protein, 11 aa, Homo sapiens
EVCIYQDTDLM

SEQ ID NO: 527 - protein, 15 aa, Homo sapiens
WCPLDLRLGSTGCLT

SEQ ID NO: 528 - DNA, 72 bp, Artificial sequence
  Codon-optimized polynucleotide
accgagatcagctgctgcaccctgagcagcgaggaaaacgagtacctgcctagacctgaatggcagctgcag

SEQ ID NO: 529 - DNA, 42 bp, Artificial sequence
  Codon-optimized polynucleotide
tgcgaggaaagaggcgcagccggatctctgatctcttgcgag

SEQ ID NO: 530 - DNA, 48 bp, Artificial sequence
  Codon-optimized polynucleotide
aacagcaagatggccctgaatagcgaggccctgtctgtggtgtccgag

SEQ ID NO: 531 - DNA, 210 bp, Artificial sequence
  Codon-optimized polynucleotide
tggggaatggaactggccgctagcaggcggtttagctgggatcatcattctgccggcggacctccaagagtgccaagcgttagaagcggagcagcccaggtccagcctaaagatccactgccactgagaacactggccggctgccttgccagaacagctcatcttagacctggcgccgaaagcctgcctcaacctcagctgcattgcaca

SEQ ID NO: 532 - DNA, 60 bp, Artificial sequence
  Codon-optimized polynucleotide
cacgttgtcggctatggccacctggatacaagcggctcctctagcagtagctcctggcct

SEQ ID NO: 533 - DNA, 105 bp, Artificial sequence
  Codon-optimized polynucleotide
aattctaagatggctctcaacagcctgaactccatcgacgacgcccagctgacaagaatcgcccctccaagaagccactgttgcttttgggaagtgaacgcccct

SEQ ID NO: 534 - DNA, 48 bp, Artificial sequence
  Codon-optimized polynucleotide
aagatgcacttctcactgaaagagcacccgccaccgccgtgcccaccg

SEQ ID NO: 535 - DNA, 207 bp, Artificial sequence
  Codon-optimized polynucleotide
ctgtggttccagtccagcgaactgtctcctactggcgctccatggccaagcagaaggcctacttggagaggcaccaccgtgtctccaagaaccgctacaagcagcgccagaacctgttgcggcacaaaatggccctccagccaagaagctgccctcggacttggaagcggactgctgagattcagctgtggcacagccgccatcaga

SEQ ID NO: 536 - DNA, 51 bp, Artificial sequence
  Codon-optimized polynucleotide
atcgccagagaactgcaccagttcgccttcgacctgctgatcaagagccac

SEQ ID NO: 537 - DNA, 51 bp, Artificial sequence
  Codon-optimized polynucleotide
cagcctgacagctttgctgccctgcatagctccctgaatgagctgggcgaa

SEQ ID NO: 538 - DNA, 51 bp, Artificial sequence
  Codon-optimized polynucleotide
tttgtgcagggtaaagattggggcctcaaaaagtttatcagacgggacttc

SEQ ID NO: 539 - DNA, 51 bp, Artificial sequence
  Codon-optimized polynucleotide
ttcgtgcagggcaaagactggggcgtgaagaagttcatccggcgggacttt

SEQ ID NO: 540 - DNA, 204 bp, Artificial sequence
  Codon-optimized polynucleotide
cagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgcctttacc

SEQ ID NO: 541 - protein, 1479 aa, Artificial seqeunce
  Heterologous protein
QNLQNGGGSRSSATLPGRRRRRWLRRRRQPISVAPAGPPRRPNQKPNPPGGARCVIMRPTWPGTSAFTKRSFAVTERIIDYWAQKEKGSSSFLRPSCDYWAQKEKISIPRTHLCLVLGVLSGHSGSRLYEAGMTLGGKILFFLFLLLPLSPFSLIFTEISCCTLSSEENEYLPRPEWQLQVPFRELKNVSVLEGLRQGRLGGPCSCHCPRPSQARLTPVDVAGPFLCLGDPGLFPPVKSSITGGKSTCSAPGPQSLPSTPFSTYPQWVILITELGMECTLGQVGAPSPRREEDGWRGGHSRFKADVPAPQGPCWGGQPGSAPSSAPPEQSLLDWKFEMSYTVGGPPPHVHARPRHWKTDRDGHSYTSKVNCLLLQDGFHGCVSITGAAGRRNLSIFLFLMLCKLEFHACKIQNKNCPDFKKFDGPCGERGGGRTARALWARGDSVLTPALDPQTPVRAPSLTRAAAAVHYKLIQQPISLFSITDRLHKTFSQLPSVHLCSITFQWGHPPIFCSTNDICVTANFCISVTFLKPCFLLHEASASQCHLFLQPQVGTPPPHTASARAPSGPPHPHESCPAGRRPARAAQTCARRQHGLPGCEEAGTARVPSLHLHLHQAALGAGRGRGWGEACAQVPPSRGVLRFLDLKVRYLHSQWQHYHRSGEAAGTPLWRPTRNVPFRELKNQRTAQGAPGIHHAASPVAANLCDPARHAQHTRIPCGAGQVRAGRGPEAGGGVLQPQRPAPEKPGCPCRRGQPRLHTVKMWRAVAMMVPDRQVHYDFGLGVPGDSTRRAVRRMNTFYEAGMTLGEKFRVGNCKHLKMTRPNSKMALNSEALSVVSECGASACDVSLIAMDSAFVQGKDWGVKKFIRRDFYAYKDFLWCFPFSLVFLQEIQICCHVSCLCCICCSTRICLGCLLELFLSRALRALHVLWNGFQLHCQTEYNQKLQVNQFSESKSLYHREKQLIAMDSAICEERGAAGSLISCETMPAILKLQKNCLLSLRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGGNTTLQQLGEASQAPSGSLIPLRLPLLWEVRGQPDSFAALHSSLNELGEIARELHQFAFDLLIKSHFVQGKDWGLKKFIRRDFWGMELAASRRFSWDHHSAGGPPRVPSVRSGAAQVQPKDPLPLRTLAGCLARTAHLRPGAESLPQPQLHCTLWFQSSELSPTGAPWPSRRPTWRGTTVSPRTATSSARTCCGTKWPSSQEAALGLGSGLLRFSCGTAAIRKMHFSLKEHPPPPCPPEAFQRAAGEGGPGRGGARRGARVLQSPFCRAGAGEWLGHQSLRHVVGYGHLDTSGSSSSSSWPNSKMALNSLNSIDDAQLTRIAPPRSHCCFWEVNAP

SEQ ID NO: 542 - DNA, 4437 bp, Artificial seqeunce
  Heterologous polynucleotide
cagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgccttcaccaagagaagctttgccgtgaccgagcggatcatcgactattgggctcaaaaagagaagggcagcagcagcttcctgcggcctagctgtgattattgggcccagaaagaaaagatcagcatccccagaacacacctgtgcctggtgctgggagtgctgtctggacactctggcagcagactgtatgaggccggcatgacactcggcggcaagatcctgttcttcctgttcctgctgctccctctgagccccttcagcctgatcttcaccgagatcagctgctgcaccctgagcagcgaggaaaacgagtacctgcctagacctgagtggcagctgcaggtccccttcagagagctgaagaacgtttccgtgctggaaggcctgagacagggcagacttggcggcccttgtagctgtcactgccccagacctagtcaggccagactgacacctgtggatgtggccggacctttcctgtgtctgggagatcctggcctgtttccacctgtgaagtccagcatcacaggcggcaagtccacatgttctgcccctggacctcagagcctgcctagcacacccttcagcacataccctcagtgggtcatcctgatcaccgaactcggcatggaatgcaccctgggacaagtgggagccccatctcctagaagagaagaggatggctggcgcggaggccactctagattcaaagctgatgtgcccgctcctcagggcccttgttggggaggacaacctggatctgccccatcttctgccccacctgaacagtccctgctggattggaagttcgagatgagctacaccgtcggcggacctccacctcatgttcatgccagacctcggcactggaaaaccgacagagatggccacagctacaccagcaaagtgaactgcctcctgctgcaggatggcttccacggctgtgtgtctattactggcgccgctggcagacggaacctgagcatctttctgtttctgatgctgtgcaagctcgagttccacgcctgcaagatccagaacaagaactgccccgacttcaagaagttcgacggcccttgcggagaaagaggcggaggcagaacagctagagccctttgggctagaggcgacagcgttctgacaccagctctggaccctcagacacctgttagggcccctagcctgacaagagctgccgccgctgtgcactacaagctgatccagcagccaatcagcctgttcagcatcaccgaccggctgcacaagacattcagccagctgccaagcgtgcacctgtgctccatcaccttccagtggggacaccctcctatcttttgctccaccaacgacatctgcgtgaccgccaacttctgtatcagcgtgaccttcctgaagccttgctttctgctgcacgaggccagcgcctctcagtgccacttgtttctgcagccccaagtgggcacacctcctccacatacagcctctgctagagcacctagcggccctccacatcctcacgaatcttgtcctgccggaagaaggcctgccagagccgctcaaacatgtgccagacgacagcacggactgcctggatgtgaagaggctggaacagccagagtgcctagcctgcacctccatctgcatcaggctgctcttggagccggaagaggtagaggatggggcgaagcttgtgctcaggtgccaccttctagaggcgtgctgagattcctggacctgaaagtgcgctacctgcacagccagtggcagcactatcacagatctggcgaagccgccggaacacccctttggaggccaacaagaaacgtgcccttccgggaactgaagaaccagagaacagctcagggcgctcctggaatccaccatgctgcttctccagtggccgccaacctgtgtgatcctgccagacatgcccagcacaccaggattccttgtggcgctggacaagtgcgcgctggaagaggacctgaagcaggcggaggtgttctgcaacctcaaagacccgctcctgagaagcctggctgcccttgcagaagaggacagcctagactgcacaccgtgaaaatgtggcgagccgtggccatgatggtgcccgatagacaggtccactacgactttggactgggcgtgccaggcgatagcactcggagagccgtcagacggatgaacaccttttacgaagccgggatgaccctgggcgagaagttcagagtgggcaactgcaagcacctgaagatgacccggcctaacagcaagatggccctgaatagcgaggccctgtctgtggtgtctgaatgtggcgcctctgcctgtgacgtgtccctgatcgctatggactccgcctttgtgcagggcaaagactggggcgtgaagaagttcatccggcgggacttctacgcctacaaggacttcctgtggtgcttccccttctctctggtgttcctgcaagagatccagatctgctgtcatgtgtcctgcctgtgctgcatctgctgtagcaccagaatctgcctgggctgtctgctggaactgttcctgagcagagccctgagagcactgcacgtgctgtggaacggattccagctgcactgccagaccgagtacaaccagaaactgcaagtgaaccagttcagcgagagcaagagcctgtaccaccgggaaaagcagctcattgccatggacagcgccatctgcgaagagagaggcgccgcaggatctctgatctcctgcgaaacaatgcccgccatcctgaagctgcagaagaattgcctcctaagcctgcgaaccgctctgacacacaaccaggacttcagcatctacagactgtgttgcaagcggggctccctgtgccatgcaagccaagctagaagccccgcctttcctaaacctgtgcgacctctgccagctccaatcaccagaattacccctcagctcggcggccagagcgattcatctcaacctctgctgaccaccggcagacctcaaggctggcaagaccaagctctgagacacacccagcaggctagccctgcctcttgtgccaccatcacaatccccatccactctgccgctctgggcgatcattctggcgatcctggaccagcctgggacacatgtcctccactgccactcacaacactgatccctagggctcctccaccttacggcgattctaccgctagaagctggcccagcagatgtggaccactcggaggcaacacaaccctccagcaactgggagaagcctctcaggctcctagcggctctctgatccctctcagactgcctctcctgtgggaagttcggggccagcctgattcttttgccgcactgcacagctccctgaacgagctgggagagatcgctagagagctgcaccagttcgccttcgacctgctgatcaagagccacttcgtgcaaggcaaggattggggcctcaaaaagtttatccgcagagacttctggggcatggaactggccgccagcagaagattcagctgggatcatcatagcgcaggcggcccacctagagtgccatctgttagaagcggagctgcccaggtgcagcctaaagatcctctgccactgagaacactggccggctgccttgctagaacagcccatcttagacctggcgccgagtctctgcctcagccacaactgcactgtaccctgtggttccagtccagcgagctgtctcctactggtgccccttggccatctagacgccctacttggagaggcaccaccgtgtcaccaagaaccgccacaagcagcgccagaacctgttgtggcacaaagtggccctccagccaagaagccgctctcggacttggaagcggactgctgaggttctcttgtggaaccgccgccattcggaagatgcactttagcctgaaagaacaccctccaccaccttgtcctccagaggctttccaaagagctgctggcgaaggcggacctggtagaggtggtgctagaagaggtgctagggtgctgcagagcccattctgtagagcaggcgcaggcgaatggctgggccatcagagtctgagacatgtcgtcggctacggccacctggatacaagcggaagcagctctagctccagctggcctaactcaaaaatggctctgaacagcctgaactccatcgacgacgcccagctgacaagaatcgcccctcctagatctcactgctgcttttgggaagtgaacgcccca

SEQ ID NO: 543 - protein, 1479 aa, Artificial seqeunce
  Hetereologous protein
QNLQNGGGSRSSATLPGRRRRRWLRRRRQPISVAPAGPPRRPNQKPNPPGGARCVIMRPTWPGTSAFTGMECTLGQVGAPSPRREEDGWRGGHSRFKADVPAPQGPCWGGQPGSAPSSAPPEQSLLDDYWAQKEKISIPRTHLCWKFEMSYTVGGPPPHVHARPRHWKTDRDYWAQKEKGSSSFLRPSCVPFRELKNVSVLEGLRQGRLGGPCSCHCPRPSQARLTPVDVAGPFLCLGDPGLFPPVKSSITEISCCTLSSEENEYLPRPEWQLQYEAGMTLGGKILFFLFLLLPLSPFSLIFLVLGVLSGHSGSRLKRSFAVTERIITGGKSTCSAPGPQSLPSTPFSTYPQWVILITELDGHSYTSKVNCLLLQDGFHGCVSITGAAGRRNLSIFLFLMLCKLEFHACQWQHYHRSGEAAGTPLWRPTRNVAMMVPDRQVHYDFGLKIQNKNCPDVLRFLDLKVRYLHSVPFRELKNQRTAQGAPGIHHAASPVAANLCDPARHAQHTRIPCGAGQVRAGRGPEAGGGVLQPQRPAPEKPGCPCRRGQPRLHTVKMWRACHLFLQPQVGTPPPHTASARAPSGPPHPHESCPAGRRPARAAQTCARRQHGLPGCEEAGTARVPSLHLHLHQAALGAGRGRGWGEACAQVPPSRGHYKLIQQPISLFSITDRLHKTFSQLPSVHLCSITFQWGHPPIFCSTNDICVTANFCISVTFLKPCFLLHEASASQFKKFDGPCGERGGGRTARALWARGDSVLTPALDPQTPVRAPSLTRAAAAVGVPGDSTRRAVRRMNTFCGASACDVSLIAMDSACEERGAAGSLISCESLYHREKQLIAMDSAIFVQGKDWGVKKFIRRDFTMPAILKLQKNCLLSLNSKMALNSEALSVVSEYEAGMTLGEKFRVGNCKHLKMTRPTEYNQKLQVNQFSESKRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGYAYKDFLWCFPFSLVFLQEIQICCHVSCLCCICCSTRICLGCLLELFLSRALRALHVLWNGFQLHCQGNTTLQQLGEASQAPSGSLIPLRLPLLWEVRGNSKMALNSLNSIDDAQLTRIAPPRSHCCFWEVNAPFVQGKDWGLKKFIRRDFEAFQRAAGEGGPGRGGARRGARVLQSPFCRAGAGEWLGHQSLRWGMELAASRRFSWDHHSAGGPPRVPSVRSGAAQVQPKDPLPLRTLAGCLARTAHLRPGAESLPQPQLHCTIARELHQFAFDLLIKSHKMHFSLKEHPPPPCPPHVVGYGHLDTSGSSSSSSWPQPDSFAALHSSLNELGELWFQSSELSPTGAPWPSRRPTWRGTTVSPRTATSSARTCCGTKWPSSQEAALGLGSGLLRFSCGTAAIR

SEQ ID NO: 544 - DNA, 4437 bp, Artificial seqeunce
  Heterologous polynucleotide
cagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgcctttaccggcatggaatgtacactgggccaagtgggagccccatctcctagaagagaagaggatggctggcgcggaggccactctagattcaaagctgatgtgcccgctcctcagggcccttgttggggaggacaacctggatctgccccatcttctgccccacctgaacagagcctgctggatgactattgggctcaaaaagagaagatcagcatccccagaacacacctgtgctggaagttcgagatgagctacaccgttggcggccctccaccacatgttcacgccagacctagacactggaaaaccgacagagattattgggcccagaaagaaaagggcagcagcagcttcctgcggcctagctgtgtgcccttccgggaactgaagaacgtgtccgttctggaaggcctgaggcagggcagacttggcggaccttgtagctgccactgtcctagaccaagccaggccagactgacccctgtggatgtggctggcccatttctgtgtctgggcgaccctggactgttccctccagtgaagtctagcatcaccgagatcagctgctgcaccctgagcagcgaggaaaacgagtacctgcctagacctgaatggcagctgcagtacgaggccggcatgacactcggaggcaagatcctgttcttcctgttcctgctgctccctctgagccccttcagcctgatctttctggtgctgggcgtgctgtctggccactctggaagcagactgaagagaagcttcgccgtgaccgagcggatcatcacaggcggcaagagcacatgttctgcccctggacctcagtctctgcccagcacacccttcagcacataccctcagtgggtcatcctgatcaccgagctggatggccacagctacaccagcaaagtgaactgcctcctgctgcaggatggcttccacggctgtgtgtctattactggcgccgctggcagacggaacctgagcatctttctgtttctgatgctgtgcaagctcgagttccacgcctgccaatggcagcactaccacagatctggcgaagccgctggaaccccactttggaggcctaccagaaacgtggccatgatggtgcccgacagacaggtgcactacgacttcggcctgaagatccagaacaagaactgccccgacgtgctgcggttcctggatctcaaagtgcgctacctgcacagcgtgcccttcagagagctgaaaaaccagagaacagcccagggcgctcctggaatccatcatgctgcttctccagtggccgccaatctgtgcgatcctgccagacatgcccagcataccaggattccttgtggcgctggacaagtgcgcgctggaagaggacctgaagctggtggcggagttctgcagcctcaaagacctgctcctgagaagcctggctgcccctgtagaagaggacagcctagactgcacaccgtgaagatgtggcgggcctgccacttgtttctccagccacaagtgggcacccctccacctcatacagcctctgctagagcacctagcggcccacctcatcctcacgaatcttgtcctgccggaagaaggcctgccagagccgctcaaacatgtgccagacgacagcacggactgcccggatgtgaagaagccggaacagccagagtgcctagcctgcaccttcatctgcatcaggccgctcttggagccggaagaggtagaggatggggagaagcttgtgcccaggtgccaccttctagaggccactacaagctgatccagcagccaatcagcctgttctccatcaccgaccggctgcacaagacattcagccagctgccttccgtgcatctgtgcagcatcaccttccagtggggacaccctcctatcttttgctccaccaacgacatctgcgtgaccgccaacttctgtatcagcgtgaccttcctgaagccttgctttctgctgcacgaggcctccgccagccagtttaagaagtttgacggcccctgcggcgagagaggcggaggaagaactgcaagagccctttgggccagaggcgactctgttctgacaccagctctggaccctcagacacctgttagggcccctagcctgacaagagctgccgctgctgttggagtgcctggcgattctactagaagggccgtgcggcggatgaacaccttttgtggcgcatctgcctgcgacgtgtccctgatcgctatggatagcgcctgcgaggaaagaggcgcagccggatctctgatctcttgcgagagcctgtaccaccgggaaaagcagctcattgccatggacagcgccatcttcgtgcagggcaaagactggggcgtgaagaagttcatccggcgggactttaccatgcctgccattctgaagctgcagaagaattgtcttctaagcctgaacagcaagatggccctgaatagcgaggccctgtctgtggtgtccgagtatgaggctggaatgaccctgggcgagaagttcagagtgggcaactgcaagcacctgaagatgacccggcctaccgagtacaaccagaaactgcaagtgaaccagttcagcgagagcaagcggaccgctctgacccacaaccaggacttcagcatctaccggctgtgctgcaagaggggctctctgtgtcatgctagccaggctagaagccccgcctttcctaagcctgtcagacctctgcctgctcctatcaccagaatcacccctcagctcggcggccagtctgattcatctcagccactgctgaccaccggcagacctcaaggatggcaagaccaggctctgagacacacacagcaggctagcccagcctcttgcgccaccatcacaataccaatacattctgccgctctgggcgatcacagcggagatcctggacctgcctgggatacttgtcctcctctgcccctaactacactgatccctagggctcctccaccttacggcgatagcacagccagatcctggcctagcagatgtggccctctgggctacgcctacaaggacttcctgtggtgcttccccttctctctggtgttcctgcaagaaatccagatctgctgtcacgtgtcctgcctgtgctgtatctgctgtagcacccggatctgtctgggctgtctgctggaactgttcctgagcagagccctgagagcactgcacgtgctgtggaacggattccagctgcactgccagggcaacaccacactgcaacagctgggagaagcctctcaggccccaagcggttctctgatccctctcagactgcccctcctgtgggaagtgcggggcaattctaagatggctctcaacagcctgaactccatcgacgacgcccagctgacaagaatcgcccctccaagaagccactgttgcttttgggaagtgaacgccccttttgtgcagggtaaagattggggcctcaaaaagtttatcagacgggacttcgaggctttccagagagcagctggcgaaggcggacctggcagaggtggtgctagaagaggtgctagagtgctgcagagcccattctgtagagctggcgctggcgaatggctgggccaccaatctcttagatggggaatggaactggccgctagcaggcggtttagctgggatcatcattctgccggcggacctccaagagtgccaagcgttagaagcggagcagcccaggtccagcctaaagatccactgccactgagaacactggccggctgccttgccagaacagctcatcttagacctggcgccgaaagcctgcctcaacctcagctgcattgcacaatcgccagagaactgcaccagttcgccttcgacctgctgatcaagagccacaagatgcacttctcactgaaagagcacccgccaccgccgtgcccaccgcacgttgtcggctatggccacctggatacaagcggctcctctagcagtagctcctggcctcagcctgacagctttgctgccctgcatagctccctgaatgagctgggcgaactgtggttccagtccagcgaactgtctcctactggcgctccatggccaagcagaaggcctacttggagaggcaccaccgtgtctccaagaaccgctacaagcagcgccagaacctgttgcggcacaaaatggccctccagccaagaagctgccctcggacttggaagcggactgctgagattcagctgtggcacagccgccatcaga

SEQ ID NO: 545 - DNA, 15 bp, Homo sapiens
cgcgacttcgccgcc

SEQ ID NO: 546 - DNA, 84 bp, Artificial seqeunce
  T cell enhancer
atgggccagaaagagcagatccacacactgcagaaaaacagcgagcggatgagcaagcagctgaccagatcttctcaggccgtg

SEQ ID NO: 547 - DNA, 21 bp, Artificial seqeunce
  Tag seqeunce
agccatcaccatcaccaccat

SEQ ID NO: 548 - protein, 12 aa, Homo sapiens
IKSLTAIFTVKF

SEQ ID NO: 549 - protein, 28 aa, Artificial sequence
  T cell enhancer
MGQKEQIHTLQKNSERMSKQLTRSSQAV

SEQ ID NO: 550 - protein, 1507 aa, Artificial seqeunce
  Heterologous polypeptide
MGQKEQIHTLQKNSERMSKQLTRSSQAVQNLQNGGGSRSSATLPGRRRRRWLRRRRQPISVAPAGPPRRPNQKPNPPGGARCVIMRPTWPGTSAFTKRSFAVTERIIDYWAQKEKGSSSFLRPSCDYWAQKEKISIPRTHLCLVLGVLSGHSGSRLYEAGMTLGGKILFFLFLLLPLSPFSLIFTEISCCTLSSEENEYLPRPEWQLQVPFRELKNVSVLEGLRQGRLGGPCSCHCPRPSQARLTPVDVAGPFLCLGDPGLFPPVKSSITGGKSTCSAPGPQSLPSTPFSTYPQWVILITELGMECTLGQVGAPSPRREEDGWRGGHSRFKADVPAPQGPCWGGQPGSAPSSAPPEQSLLDWKFEMSYTVGGPPPHVHARPRHWKTDRDGHSYTSKVNCLLLQDGFHGCVSITGAAGRRNLSIFLFLMLCKLEFHACKIQNKNCPDFKKFDGPCGERGGGRTARALWARGDSVLTPALDPQTPVRAPSLTRAAAAVHYKLIQQPISLFSITDRLHKTFSQLPSVHLCSITFQWGHPPIFCSTNDICVTANFCISVTFLKPCFLLHEASASQCHLFLQPQVGTPPPHTASARAPSGPPHPHESCPAGRRPARAAQTCARRQHGLPGCEEAGTARVPSLHLHLHQAALGAGRGRGWGEACAQVPPSRGVLRFLDLKVRYLHSQWQHYHRSGEAAGTPLWRPTRNVPFRELKNQRTAQGAPGIHHAASPVAANLCDPARHAQHTRIPCGAGQVRAGRGPEAGGGVLQPQRPAPEKPGCPCRRGQPRLHTVKMWRAVAMMVPDRQVHYDFGLGVPGDSTRRAVRRMNTFYEAGMTLGEKFRVGNCKHLKMTRPNSKMALNSEALSVVSECGASACDVSLIAMDSAFVQGKDWGVKKFIRRDFYAYKDFLWCFPFSLVFLQEIQICCHVSCLCCICCSTRICLGCLLELFLSRALRALHVLWNGFQLHCQTEYNQKLQVNQFSESKSLYHREKQLIAMDSAICEERGAAGSLISCETMPAILKLQKNCLLSLRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGGNTTLQQLGEASQAPSGSLIPLRLPLLWEVRGQPDSFAALHSSLNELGEIARELHQFAFDLLIKSHFVQGKDWGLKKFIRRDFWGMELAASRRFSWDHHSAGGPPRVPSVRSGAAQVQPKDPLPLRTLAGCLARTAHLRPGAESLPQPQLHCTLWFQSSELSPTGAPWPSRRPTWRGTTVSPRTATSSARTCCGTKWPSSQEAALGLGSGLLRFSCGTAAIRKMHFSLKEHPPPPCPPEAFQRAAGEGGPGRGGARRGARVLQSPFCRAGAGEWLGHQSLRHVVGYGHLDTSGSSSSSSWPNSKMALNSLNSIDDAQLTRIAPPRSHCCFWEVNAP

SEQ ID NO: 551 - protein, 5703 aa, Artificial seqeunce
  Heterologous polypeptide
CCATTGCATACGTTGTATCCATATCATAATATGTACATTTATATTGGCTCATGTCCAACATTACCGCCATGTTGACATTGATTATTGACTAGTTATTAATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCAATAATGACGTATGTTCCCATAGTAACGCCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTAAATGGCCCGCCTGGCATTATGCCCAGTACATGACCTTATGGGACTTTCCTACTTGGCAGTACATCTACGTATTAGTCATCGCTATTACCATGGTGATGCGGTTTTGGCAGTACATCAATGGGCGTGGATAGCGGTTTGACTCACGGGGATTTCCAAGTCTCCACCCCATTGACGTCAATGGGAGTTTGTTTTGGCACCAAAATCAACGGGACTTTCCAAAATGTCGTAACAACTCCGCCCCATTGACGCAAATGGGCGGTAGGCGTGTACGGTGGGAGGTCTATATAAGCAGAGCTCTCCCTATCAGTGATAGAGATCTCCCTATCAGTGATAGAGATCGTCGACGAGCTCGTTTAGTGAACCGTCAGATCGCCTGGAGACGCCATCCACGCTGTTTTGACCTCCATAGAAGACACCGGGACCGATCCAGCCTCCGCGGCCGGGAACGGTGCATTGGAACGCGGATTCCCCGTGCCAAGAGTGAGATCTTCCGTTTATCTAGGTACCAGATATCAGAATTCGGATCCCGCGACTTCGCCGCCATGGGCCAGAAAGAGCAGATCCACACACTGCAGAAAAACAGCGAGCGGATGAGCAAGCAGCTGACCAGATCTTCTCAGGCCGTGCAGAACCTGCAGAACGGCGGAGGCTCTAGAAGCTCTGCTACACTTCCTGGCAGGCGGCGGAGAAGATGGCTGAGAAGAAGGCGGCAGCCTATCTCTGTGGCTCCTGCTGGACCTCCTAGACGGCCCAACCAGAAGCCTAATCCTCCTGGCGGAGCCAGATGCGTGATCATGAGGCCTACATGGCCTGGCACCAGCGCCTTCACCAAGAGAAGCTTTGCCGTGACCGAGCGGATCATCGACTATTGGGCTCAAAAAGAGAAGGGCAGCAGCAGCTTCCTGCGGCCTAGCTGTGATTATTGGGCCCAGAAAGAAAAGATCAGCATCCCCAGAACACACCTGTGCCTGGTGCTGGGAGTGCTGTCTGGACACTCTGGCAGCAGACTGTATGAGGCCGGCATGACACTCGGCGGCAAGATCCTGTTCTTCCTGTTCCTGCTGCTCCCTCTGAGCCCCTTCAGCCTGATCTTCACCGAGATCAGCTGCTGCACCCTGAGCAGCGAGGAAAACGAGTACCTGCCTAGACCTGAGTGGCAGCTGCAGGTCCCCTTCAGAGAGCTGAAGAACGTTTCCGTGCTGGAAGGCCTGAGACAGGGCAGACTTGGCGGCCCTTGTAGCTGTCACTGCCCCAGACCTAGTCAGGCCAGACTGACACCTGTGGATGTGGCCGGACCTTTCCTGTGTCTGGGAGATCCTGGCCTGTTTCCACCTGTGAAGTCCAGCATCACAGGCGGCAAGTCCACATGTTCTGCCCCTGGACCTCAGAGCCTGCCTAGCACACCCTTCAGCACATACCCTCAGTGGGTCATCCTGATCACCGAACTCGGCATGGAATGCACCCTGGGACAAGTGGGAGCCCCATCTCCTAGAAGAGAAGAGGATGGCTGGCGCGGAGGCCACTCTAGATTCAAAGCTGATGTGCCCGCTCCTCAGGGCCCTTGTTGGGGAGGACAACCTGGATCTGCCCCATCTTCTGCCCCACCTGAACAGTCCCTGCTGGATTGGAAGTTCGAGATGAGCTACACCGTCGGCGGACCTCCACCTCATGTTCATGCCAGACCTCGGCACTGGAAAACCGACAGAGATGGCCACAGCTACACCAGCAAAGTGAACTGCCTCCTGCTGCAGGATGGCTTCCACGGCTGTGTGTCTATTACTGGCGCCGCTGGCAGACGGAACCTGAGCATCTTTCTGTTTCTGATGCTGTGCAAGCTCGAGTTCCACGCCTGCAAGATCCAGAACAAGAACTGCCCCGACTTCAAGAAGTTCGACGGCCCTTGCGGAGAAAGAGGCGGAGGCAGAACAGCTAGAGCCCTTTGGGCTAGAGGCGACAGCGTTCTGACACCAGCTCTGGACCCTCAGACACCTGTTAGGGCCCCTAGCCTGACAAGAGCTGCCGCCGCTGTGCACTACAAGCTGATCCAGCAGCCAATCAGCCTGTTCAGCATCACCGACCGGCTGCACAAGACATTCAGCCAGCTGCCAAGCGTGCACCTGTGCTCCATCACCTTCCAGTGGGGACACCCTCCTATCTTTTGCTCCACCAACGACATCTGCGTGACCGCCAACTTCTGTATCAGCGTGACCTTCCTGAAGCCTTGCTTTCTGCTGCACGAGGCCAGCGCCTCTCAGTGCCACTTGTTTCTGCAGCCCCAAGTGGGCACACCTCCTCCACATACAGCCTCTGCTAGAGCACCTAGCGGCCCTCCACATCCTCACGAATCTTGTCCTGCCGGAAGAAGGCCTGCCAGAGCCGCTCAAACATGTGCCAGACGACAGCACGGACTGCCTGGATGTGAAGAGGCTGGAACAGCCAGAGTGCCTAGCCTGCACCTCCATCTGCATCAGGCTGCTCTTGGAGCCGGAAGAGGTAGAGGATGGGGCGAAGCTTGTGCTCAGGTGCCACCTTCTAGAGGCGTGCTGAGATTCCTGGACCTGAAAGTGCGCTACCTGCACAGCCAGTGGCAGCACTATCACAGATCTGGCGAAGCCGCCGGAACACCCCTTTGGAGGCCAACAAGAAACGTGCCCTTCCGGGAACTGAAGAACCAGAGAACAGCTCAGGGCGCTCCTGGAATCCACCATGCTGCTTCTCCAGTGGCCGCCAACCTGTGTGATCCTGCCAGACATGCCCAGCACACCAGGATTCCTTGTGGCGCTGGACAAGTGCGCGCTGGAAGAGGACCTGAAGCAGGCGGAGGTGTTCTGCAACCTCAAAGACCCGCTCCTGAGAAGCCTGGCTGCCCTTGCAGAAGAGGACAGCCTAGACTGCACACCGTGAAAATGTGGCGAGCCGTGGCCATGATGGTGCCCGATAGACAGGTCCACTACGACTTTGGACTGGGCGTGCCAGGCGATAGCACTCGGAGAGCCGTCAGACGGATGAACACCTTTTACGAAGCCGGGATGACCCTGGGCGAGAAGTTCAGAGTGGGCAACTGCAAGCACCTGAAGATGACCCGGCCTAACAGCAAGATGGCCCTGAATAGCGAGGCCCTGTCTGTGGTGTCTGAATGTGGCGCCTCTGCCTGTGACGTGTCCCTGATCGCTATGGACTCCGCCTTTGTGCAGGGCAAAGACTGGGGCGTGAAGAAGTTCATCCGGCGGGACTTCTACGCCTACAAGGACTTCCTGTGGTGCTTCCCCTTCTCTCTGGTGTTCCTGCAAGAGATCCAGATCTGCTGTCATGTGTCCTGCCTGTGCTGCATCTGCTGTAGCACCAGAATCTGCCTGGGCTGTCTGCTGGAACTGTTCCTGAGCAGAGCCCTGAGAGCACTGCACGTGCTGTGGAACGGATTCCAGCTGCACTGCCAGACCGAGTACAACCAGAAACTGCAAGTGAACCAGTTCAGCGAGAGCAAGAGCCTGTACCACCGGGAAAAGCAGCTCATTGCCATGGACAGCGCCATCTGCGAAGAGAGAGGCGCCGCAGGATCTCTGATCTCCTGCGAAACAATGCCCGCCATCCTGAAGCTGCAGAAGAATTGCCTCCTAAGCCTGCGAACCGCTCTGACACACAACCAGGACTTCAGCATCTACAGACTGTGTTGCAAGCGGGGCTCCCTGTGCCATGCAAGCCAAGCTAGAAGCCCCGCCTTTCCTAAACCTGTGCGACCTCTGCCAGCTCCAATCACCAGAATTACCCCTCAGCTCGGCGGCCAGAGCGATTCATCTCAACCTCTGCTGACCACCGGCAGACCTCAAGGCTGGCAAGACCAAGCTCTGAGACACACCCAGCAGGCTAGCCCTGCCTCTTGTGCCACCATCACAATCCCCATCCACTCTGCCGCTCTGGGCGATCATTCTGGCGATCCTGGACCAGCCTGGGACACATGTCCTCCACTGCCACTCACAACACTGATCCCTAGGGCTCCTCCACCTTACGGCGATTCTACCGCTAGAAGCTGGCCCAGCAGATGTGGACCACTCGGAGGCAACACAACCCTCCAGCAACTGGGAGAAGCCTCTCAGGCTCCTAGCGGCTCTCTGATCCCTCTCAGACTGCCTCTCCTGTGGGAAGTTCGGGGCCAGCCTGATTCTTTTGCCGCACTGCACAGCTCCCTGAACGAGCTGGGAGAGATCGCTAGAGAGCTGCACCAGTTCGCCTTCGACCTGCTGATCAAGAGCCACTTCGTGCAAGGCAAGGATTGGGGCCTCAAAAAGTTTATCCGCAGAGACTTCTGGGGCATGGAACTGGCCGCCAGCAGAAGATTCAGCTGGGATCATCATAGCGCAGGCGGCCCACCTAGAGTGCCATCTGTTAGAAGCGGAGCTGCCCAGGTGCAGCCTAAAGATCCTCTGCCACTGAGAACACTGGCCGGCTGCCTTGCTAGAACAGCCCATCTTAGACCTGGCGCCGAGTCTCTGCCTCAGCCACAACTGCACTGTACCCTGTGGTTCCAGTCCAGCGAGCTGTCTCCTACTGGTGCCCCTTGGCCATCTAGACGCCCTACTTGGAGAGGCACCACCGTGTCACCAAGAACCGCCACAAGCAGCGCCAGAACCTGTTGTGGCACAAAGTGGCCCTCCAGCCAAGAAGCCGCTCTCGGACTTGGAAGCGGACTGCTGAGGTTCTCTTGTGGAACCGCCGCCATTCGGAAGATGCACTTTAGCCTGAAAGAACACCCTCCACCACCTTGTCCTCCAGAGGCTTTCCAAAGAGCTGCTGGCGAAGGCGGACCTGGTAGAGGTGGTGCTAGAAGAGGTGCTAGGGTGCTGCAGAGCCCATTCTGTAGAGCAGGCGCAGGCGAATGGCTGGGCCATCAGAGTCTGAGACATGTCGTCGGCTACGGCCACCTGGATACAAGCGGAAGCAGCTCTAGCTCCAGCTGGCCTAACTCAAAAATGGCTCTGAACAGCCTGAACTCCATCGACGACGCCCAGCTGACAAGAATCGCCCCTCCTAGATCTCACTGCTGCTTTTGGGAAGTGAACGCCCCAAGCCATCACCATCACCACCATTAGTAAAGGCGCGCCTAGCGGCCGCGATCTGCTGTGCCTTCTAGTTGCCAGCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTGAGTAGGTGTCATTCTATTCTGGGGGGTGGGGTGGGGCAGGACAGCAAGGGGGAGGATTGGGAAGACAATAGCAGGCATGCTGGGGATGCGGTGGGCTCTATGGCC

SEQ ID NO: 552 - protein, 1507 aa, Artificial seqeunce
  Heterologous polypeptide
MGQKEQIHTLQKNSERMSKQLTRSSQAVQNLQNGGGSRSSATLPGRRRRRWLRRRRQPISVAPAGPPRRPNQKPNPPGGARCVIMRPTWPGTSAFTGMECTLGQVGAPSPRREEDGWRGGHSRFKADVPAPQGPCWGGQPGSAPSSAPPEQSLLDDYWAQKEKISIPRTHLCWKFEMSYTVGGPPPHVHARPRHWKTDRDYWAQKEKGSSSFLRPSCVPFRELKNVSVLEGLRQGRLGGPCSCHCPRPSQARLTPVDVAGPFLCLGDPGLFPPVKSSITEISCCTLSSEENEYLPRPEWQLQYEAGMTLGGKILFFLFLLLPLSPFSLIFLVLGVLSGHSGSRLKRSFAVTERIITGGKSTCSAPGPQSLPSTPFSTYPQWVILITELDGHSYTSKVNCLLLQDGFHGCVSITGAAGRRNLSIFLFLMLCKLEFHACQWQHYHRSGEAAGTPLWRPTRNVAMMVPDRQVHYDFGLKIQNKNCPDVLRFLDLKVRYLHSVPFRELKNQRTAQGAPGIHHAASPVAANLCDPARHAQHTRIPCGAGQVRAGRGPEAGGGVLQPQRPAPEKPGCPCRRGQPRLHTVKMWRACHLFLQPQVGTPPPHTASARAPSGPPHPHESCPAGRRPARAAQTCARRQHGLPGCEEAGTARVPSLHLHLHQAALGAGRGRGWGEACAQVPPSRGHYKLIQQPISLFSITDRLHKTFSQLPSVHLCSITFQWGHPPIFCSTNDICVTANFCISVTFLKPCFLLHEASASQFKKFDGPCGERGGGRTARALWARGDSVLTPALDPQTPVRAPSLTRAAAAVGVPGDSTRRAVRRMNTFCGASACDVSLIAMDSACEERGAAGSLISCESLYHREKQLIAMDSAIFVQGKDWGVKKFIRRDFTMPAILKLQKNCLLSLNSKMALNSEALSVVSEYEAGMTLGEKFRVGNCKHLKMTRPTEYNQKLQVNQFSESKRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGYAYKDFLWCFPFSLVFLQEIQICCHVSCLCCICCSTRICLGCLLELFLSRALRALHVLWNGFQLHCQGNTTLQQLGEASQAPSGSLIPLRLPLLWEVRGNSKMALNSLNSIDDAQLTRIAPPRSHCCFWEVNAPFVQGKDWGLKKFIRRDFEAFQRAAGEGGPGRGGARRGARVLQSPFCRAGAGEWLGHQSLRWGMELAASRRFSWDHHSAGGPPRVPSVRSGAAQVQPKDPLPLRTLAGCLARTAHLRPGAESLPQPQLHCTIARELHQFAFDLLIKSHKMHFSLKEHPPPPCPPHVVGYGHLDTSGSSSSSSWPQPDSFAALHSSLNELGELWFQSSELSPTGAPWPSRRPTWRGTTVSPRTATSSARTCCGTKWPSSQEAALGLGSGLLRFSCGTAAIR

SEQ ID NO: 553 - DNA, 4722 bp, Artificial seqeunce
  Heterologous polynucleotide
gatcactaattccaaacccacccgctttttatagtaagtttttcacccataaataataaatacaataattaatttctcgtaaaagtagaaaatatattctaatttattgcacggtaaggaagtagaatcataaagaacagtgacggatcccgcgacttcgccgccatgggccagaaagagcagatccacacactgcagaaaaacagcgagcggatgagcaagcagctgaccagatcttctcaggccgtgcagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgcctttaccggcatggaatgtacactgggccaagtgggagccccatctcctagaagagaagaggatggctggcgcggaggccactctagattcaaagctgatgtgcccgctcctcagggcccttgttggggaggacaacctggatctgccccatcttctgccccacctgaacagagcctgctggatgactattgggctcaaaaagagaagatcagcatccccagaacacacctgtgctggaagttcgagatgagctacaccgttggcggccctccaccacatgttcacgccagacctagacactggaaaaccgacagagattattgggcccagaaagaaaagggcagcagcagcttcctgcggcctagctgtgtgcccttccgggaactgaagaacgtgtccgttctggaaggcctgaggcagggcagacttggcggaccttgtagctgccactgtcctagaccaagccaggccagactgacccctgtggatgtggctggcccatttctgtgtctgggcgaccctggactgttccctccagtgaagtctagcatcaccgagatcagctgctgcaccctgagcagcgaggaaaacgagtacctgcctagacctgaatggcagctgcagtacgaggccggcatgacactcggaggcaagatcctgttcttcctgttcctgctgctccctctgagccccttcagcctgatctttctggtgctgggcgtgctgtctggccactctggaagcagactgaagagaagcttcgccgtgaccgagcggatcatcacaggcggcaagagcacatgttctgcccctggacctcagtctctgcccagcacacccttcagcacataccctcagtgggtcatcctgatcaccgagctggatggccacagctacaccagcaaagtgaactgcctcctgctgcaggatggcttccacggctgtgtgtctattactggcgccgctggcagacggaacctgagcatctttctgtttctgatgctgtgcaagctcgagttccacgcctgccaatggcagcactaccacagatctggcgaagccgctggaaccccactttggaggcctaccagaaacgtggccatgatggtgcccgacagacaggtgcactacgacttcggcctgaagatccagaacaagaactgccccgacgtgctgcggttcctggatctcaaagtgcgctacctgcacagcgtgcccttcagagagctgaaaaaccagagaacagcccagggcgctcctggaatccatcatgctgcttctccagtggccgccaatctgtgcgatcctgccagacatgcccagcataccaggattccttgtggcgctggacaagtgcgcgctggaagaggacctgaagctggtggcggagttctgcagcctcaaagacctgctcctgagaagcctggctgcccctgtagaagaggacagcctagactgcacaccgtgaagatgtggcgggcctgccacttgtttctccagccacaagtgggcacccctccacctcatacagcctctgctagagcacctagcggcccacctcatcctcacgaatcttgtcctgccggaagaaggcctgccagagccgctcaaacatgtgccagacgacagcacggactgcccggatgtgaagaagccggaacagccagagtgcctagcctgcaccttcatctgcatcaggccgctcttggagccggaagaggtagaggatggggagaagcttgtgcccaggtgccaccttctagaggccactacaagctgatccagcagccaatcagcctgttctccatcaccgaccggctgcacaagacattcagccagctgccttccgtgcatctgtgcagcatcaccttccagtggggacaccctcctatcttttgctccaccaacgacatctgcgtgaccgccaacttctgtatcagcgtgaccttcctgaagccttgctttctgctgcacgaggcctccgccagccagtttaagaagtttgacggcccctgcggcgagagaggcggaggaagaactgcaagagccctttgggccagaggcgactctgttctgacaccagctctggaccctcagacacctgttagggcccctagcctgacaagagctgccgctgctgttggagtgcctggcgattctactagaagggccgtgcggcggatgaacaccttttgtggcgcatctgcctgcgacgtgtccctgatcgctatggatagcgcctgcgaggaaagaggcgcagccggatctctgatctcttgcgagagcctgtaccaccgggaaaagcagctcattgccatggacagcgccatcttcgtgcagggcaaagactggggcgtgaagaagttcatccggcgggactttaccatgcctgccattctgaagctgcagaagaattgtcttctaagcctgaacagcaagatggccctgaatagcgaggccctgtctgtggtgtccgagtatgaggctggaatgaccctgggcgagaagttcagagtgggcaactgcaagcacctgaagatgacccggcctaccgagtacaaccagaaactgcaagtgaaccagttcagcgagagcaagcggaccgctctgacccacaaccaggacttcagcatctaccggctgtgctgcaagaggggctctctgtgtcatgctagccaggctagaagccccgcctttcctaagcctgtcagacctctgcctgctcctatcaccagaatcacccctcagctcggcggccagtctgattcatctcagccactgctgaccaccggcagacctcaaggatggcaagaccaggctctgagacacacacagcaggctagcccagcctcttgcgccaccatcacaataccaatacattctgccgctctgggcgatcacagcggagatcctggacctgcctgggatacttgtcctcctctgcccctaactacactgatccctagggctcctccaccttacggcgatagcacagccagatcctggcctagcagatgtggccctctgggctacgcctacaaggacttcctgtggtgcttccccttctctctggtgttcctgcaagaaatccagatctgctgtcacgtgtcctgcctgtgctgtatctgctgtagcacccggatctgtctgggctgtctgctggaactgttcctgagcagagccctgagagcactgcacgtgctgtggaacggattccagctgcactgccagggcaacaccacactgcaacagctgggagaagcctctcaggccccaagcggttctctgatccctctcagactgcccctcctgtgggaagtgcggggcaattctaagatggctctcaacagcctgaactccatcgacgacgcccagctgacaagaatcgcccctccaagaagccactgttgcttttgggaagtgaacgccccttttgtgcagggtaaagattggggcctcaaaaagtttatcagacgggacttcgaggctttccagagagcagctggcgaaggcggacctggcagaggtggtgctagaagaggtgctagagtgctgcagagcccattctgtagagctggcgctggcgaatggctgggccaccaatctcttagatggggaatggaactggccgctagcaggcggtttagctgggatcatcattctgccggcggacctccaagagtgccaagcgttagaagcggagcagcccaggtccagcctaaagatccactgccactgagaacactggccggctgccttgccagaacagctcatcttagacctggcgccgaaagcctgcctcaacctcagctgcattgcacaatcgccagagaactgcaccagttcgccttcgacctgctgatcaagagccacaagatgcacttctcactgaaagagcacccgccaccgccgtgcccaccgcacgttgtcggctatggccacctggatacaagcggctcctctagcagtagctcctggcctcagcctgacagctttgctgccctgcatagctccctgaatgagctgggcgaactgtggttccagtccagcgaactgtctcctactggcgctccatggccaagcagaaggcctacttggagaggcaccaccgtgtctccaagaaccgctacaagcagcgccagaacctgttgcggcacaaaatggccctccagccaagaagctgccctcggacttggaagcggactgctgagattcagctgtggcacagccgccatcagaagccatcaccatcaccaccattagtaaaggcgcgcc

SEQ ID NO: 554 - protein, 1507 aa, Artificial seqeunce
  Heterologous polypeptide
MGQKEQIHTLQKNSERMSKQLTRSSQAVQNLQNGGGSRSSATLPGRRRRRWLRRRRQPISVAPAGPPRRPNQKPNPPGGARCVIMRPTWPGTSAFTKRSFAVTERIILVLGVLSGHSGSRLYEAGMTLGGKILFFLFLLLPLSPFSLIFTEISCCTLSSEENEYLPRPEWQLQVPFRELKNVSVLEGLRQGRLGGPCSCHCPRPSQARLTPVDVAGPFLCLGDPGLFPPVKSSITGGKSTCSAPGPQSLPSTPFSTYPQWVILITELGMECTLGQVGAPSPRREEDGWRGGHSRFKADVPAPQGPCWGGQPGSAPSSAPPEQSLLDDYWAQKEKGSSSFLRPSCDYWAQKEKISIPRTHLCWKFEMSYTVGGPPPHVHARPRHWKTDRGVPGDSTRRAVRRMNTFYEAGMTLGEKFRVGNCKHLKMTRPNSKMALNSEALSVVSECGASACDVSLIAMDSAFVQGKDWGVKKFIRRDFYAYKDFLWCFPFSLVFLQEIQICCHVSCLCCICCSTRICLGCLLELFLSRALRALHVLWNGFQLHCQTEYNQKLQVNQFSESKSLYHREKQLIAMDSAICEERGAAGSLISCETMPAILKLQKNCLLSLRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGDGHSYTSKVNCLLLQDGFHGCVSITGAAGRRNLSIFLFLMLCKLEFHACKIQNKNCPDFKKFDGPCGERGGGRTARALWARGDSVLTPALDPQTPVRAPSLTRAAAAVHYKLIQQPISLFSITDRLHKTFSQLPSVHLCSITFQWGHPPIFCSTNDICVTANFCISVTFLKPCFLLHEASASQCHLFLQPQVGTPPPHTASARAPSGPPHPHESCPAGRRPARAAQTCARRQHGLPGCEEAGTARVPSLHLHLHQAALGAGRGRGWGEACAQVPPSRGVLRFLDLKVRYLHSQWQHYHRSGEAAGTPLWRPTRNVPFRELKNQRTAQGAPGIHHAASPVAANLCDPARHAQHTRIPCGAGQVRAGRGPEAGGGVLQPQRPAPEKPGCPCRRGQPRLHTVKMWRAVAMMVPDRQVHYDFGLGNTTLQQLGEASQAPSGSLIPLRLPLLWEVRGQPDSFAALHSSLNELGEIARELHQFAFDLLIKSHFVQGKDWGLKKFIRRDFWGMELAASRRFSWDHHSAGGPPRVPSVRSGAAQVQPKDPLPLRTLAGCLARTAHLRPGAESLPQPQLHCTLWFQSSELSPTGAPWPSRRPTWRGTTVSPRTATSSARTCCGTKWPSSQEAALGLGSGLLRFSCGTAAIRKMHFSLKEHPPPPCPPEAFQRAAGEGGPGRGGARRGARVLQSPFCRAGAGEWLGHQSLRHVVGYGHLDTSGSSSSSSWPNSKMALNSLNSIDDAQLTRIAPPRSHCCFWEVNAP

SEQ ID NO: 555 - protein, 1507 aa, Artificial seqeunce
  Heterologous polypeptide
MGQKEQIHTLQKNSERMSKQLTRSSQAVQNLQNGGGSRSSATLPGRRRRRWLRRRRQPISVAPAGPPRRPNQKPNPPGGARCVIMRPTWPGTSAFTKRSFAVTERIITEISCCTLSSEENEYLPRPEWQLQYEAGMTLGGKILFFLFLLLPLSPFSLIFDYWAQKEKGSSSFLRPSCVPFRELKNVSVLEGLRQGRLGGPCSCHCPRPSQARLTPVDVAGPFLCLGDPGLFPPVKSSITGGKSTCSAPGPQSLPSTPFSTYPQWVILITELGMECTLGQVGAPSPRREEDGWRGGHSRFKADVPAPQGPCWGGQPGSAPSSAPPEQSLLDDYWAQKEKISIPRTHLCLVLGVLSGHSGSRLWKFEMSYTVGGPPPHVHARPRHWKTDRDGHSYTSKVNCLLLQDGFHGCVSITGAAGRRNLSIFLFLMLCKLEFHACKIQNKNCPDFKKFDGPCGERGGGRTARALWARGDSVLTPALDPQTPVRAPSLTRAAAAVHYKLIQQPISLFSITDRLHKTFSQLPSVHLCSITFQWGHPPIFCSTNDICVTANFCISVTFLKPCFLLHEASASQCHLFLQPQVGTPPPHTASARAPSGPPHPHESCPAGRRPARAAQTCARRQHGLPGCEEAGTARVPSLHLHLHQAALGAGRGRGWGEACAQVPPSRGVLRFLDLKVRYLHSQWQHYHRSGEAAGTPLWRPTRNVPFRELKNQRTAQGAPGIHHAASPVAANLCDPARHAQHTRIPCGAGQVRAGRGPEAGGGVLQPQRPAPEKPGCPCRRGQPRLHTVKMWRAVAMMVPDRQVHYDFGLGVPGDSTRRAVRRMNTFYEAGMTLGEKFRVGNCKHLKMTRPNSKMALNSEALSVVSECGASACDVSLIAMDSAFVQGKDWGVKKFIRRDFYAYKDFLWCFPFSLVFLQEIQICCHVSCLCCICCSTRICLGCLLELFLSRALRALHVLWNGFQLHCQTEYNQKLQVNQFSESKSLYHREKQLIAMDSAICEERGAAGSLISCETMPAILKLQKNCLLSLRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGGNTTLQQLGEASQAPSGSLIPLRLPLLWEVRGQPDSFAALHSSLNELGEIARELHQFAFDLLIKSHFVQGKDWGLKKFIRRDFWGMELAASRRFSWDHHSAGGPPRVPSVRSGAAQVQPKDPLPLRTLAGCLARTAHLRPGAESLPQPQLHCTLWFQSSELSPTGAPWPSRRPTWRGTTVSPRTATSSARTCCGTKWPSSQEAALGLGSGLLRFSCGTAAIRKMHFSLKEHPPPPCPPEAFQRAAGEGGPGRGGARRGARVLQSPFCRAGAGEWLGHQSLRHVVGYGHLDTSGSSSSSSWPNSKMALNSLNSIDDAQLTRIAPPRSHCCFWEVNAP

SEQ ID NO: 556 - protein, 1507 aa, Artificial seqeunce
  Heterologous polypeptide
MGQKEQIHTLQKNSERMSKQLTRSSQAVQNLQNGGGSRSSATLPGRRRRRWLRRRRQPISVAPAGPPRRPNQKPNPPGGARCVIMRPTWPGTSAFTKRSFAVTERIIDYWAQKEKGSSSFLRPSCVPFRELKNVSVLEGLRQGRLGGPCSCHCPRPSQARLTPVDVAGPFLCLGDPGLFPPVKSSILVLGVLSGHSGSRLYEAGMTLGGKILFFLFLLLPLSPFSLIFTEISCCTLSSEENEYLPRPEWQLQTGGKSTCSAPGPQSLPSTPFSTYPQWVILITELGMECTLGQVGAPSPRREEDGWRGGHSRFKADVPAPQGPCWGGQPGSAPSSAPPEQSLLDDYWAQKEKISIPRTHLCWKFEMSYTVGGPPPHVHARPRHWKTDRDGHSYTSKVNCLLLQDGFHGCVSITGAAGRRNLSIFLFLMLCKLEFHACKIQNKNCPDFKKFDGPCGERGGGRTARALWARGDSVLTPALDPQTPVRAPSLTRAAAAVHYKLIQQPISLFSITDRLHKTFSQLPSVHLCSITFQWGHPPIFCSTNDICVTANFCISVTFLKPCFLLHEASASQCHLFLQPQVGTPPPHTASARAPSGPPHPHESCPAGRRPARAAQTCARRQHGLPGCEEAGTARVPSLHLHLHQAALGAGRGRGWGEACAQVPPSRGVLRFLDLKVRYLHSQWQHYHRSGEAAGTPLWRPTRNVPFRELKNQRTAQGAPGIHHAASPVAANLCDPARHAQHTRIPCGAGQVRAGRGPEAGGGVLQPQRPAPEKPGCPCRRGQPRLHTVKMWRAVAMMVPDRQVHYDFGLGVPGDSTRRAVRRMNTFYEAGMTLGEKFRVGNCKHLKMTRPNSKMALNSEALSVVSECGASACDVSLIAMDSAFVQGKDWGVKKFIRRDFYAYKDFLWCFPFSLVFLQEIQICCHVSCLCCICCSTRICLGCLLELFLSRALRALHVLWNGFQLHCQTEYNQKLQVNQFSESKSLYHREKQLIAMDSAICEERGAAGSLISCETMPAILKLQKNCLLSLRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGGNTTLQQLGEASQAPSGSLIPLRLPLLWEVRGQPDSFAALHSSLNELGEIARELHQFAFDLLIKSHFVQGKDWGLKKFIRRDFWGMELAASRRFSWDHHSAGGPPRVPSVRSGAAQVQPKDPLPLRTLAGCLARTAHLRPGAESLPQPQLHCTLWFQSSELSPTGAPWPSRRPTWRGTTVSPRTATSSARTCCGTKWPSSQEAALGLGSGLLRFSCGTAAIRKMHFSLKEHPPPPCPPEAFQRAAGEGGPGRGGARRGARVLQSPFCRAGAGEWLGHQSLRHVVGYGHLDTSGSSSSSSWPNSKMALNSLNSIDDAQLTRIAPPRSHCCFWEVNAP

SEQ ID NO: 557 - protein, 1507 aa, Artificial seqeunce
  Heterologous polypeptide
MGQKEQIHTLQKNSERMSKQLTRSSQAVQNLQNGGGSRSSATLPGRRRRRWLRRRRQPISVAPAGPPRRPNQKPNPPGGARCVIMRPTWPGTSAFTGMECTLGQVGAPSPRREEDGWRGGHSRFKADVPAPQGPCWGGQPGSAPSSAPPEQSLLDDYWAQKEKISIPRTHLCWKFEMSYTVGGPPPHVHARPRHWKTDRDYWAQKEKGSSSFLRPSCVPFRELKNVSVLEGLRQGRLGGPCSCHCPRPSQARLTPVDVAGPFLCLGDPGLFPPVKSSITEISCCTLSSEENEYLPRPEWQLQYEAGMTLGGKILFFLFLLLPLSPFSLIFLVLGVLSGHSGSRLKRSFAVTERIITGGKSTCSAPGPQSLPSTPFSTYPQWVILITELDGHSYTSKVNCLLLQDGFHGCVSITGAAGRRNLSIFLFLMLCKLEFHACCHLFLQPQVGTPPPHTASARAPSGPPHPHESCPAGRRPARAAQTCARRQHGLPGCEEAGTARVPSLHLHLHQAALGAGRGRGWGEACAQVPPSRGHYKLIQQPISLFSITDRLHKTFSQLPSVHLCSITFQWGHPPIFCSTNDICVTANFCISVTFLKPCFLLHEASASQVAMMVPDRQVHYDFGLKIQNKNCPDVLRFLDLKVRYLHSVPFRELKNQRTAQGAPGIHHAASPVAANLCDPARHAQHTRIPCGAGQVRAGRGPEAGGGVLQPQRPAPEKPGCPCRRGQPRLHTVKMWRAQWQHYHRSGEAAGTPLWRPTRNFKKFDGPCGERGGGRTARALWARGDSVLTPALDPQTPVRAPSLTRAAAAVGVPGDSTRRAVRRMNTFCGASACDVSLIAMDSACEERGAAGSLISCESLYHREKQLIAMDSAIFVQGKDWGVKKFIRRDFTMPAILKLQKNCLLSLNSKMALNSEALSVVSEYEAGMTLGEKFRVGNCKHLKMTRPTEYNQKLQVNQFSESKRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGYAYKDFLWCFPFSLVFLQEIQICCHVSCLCCICCSTRICLGCLLELFLSRALRALHVLWNGFQLHCQGNTTLQQLGEASQAPSGSLIPLRLPLLWEVRGNSKMALNSLNSIDDAQLTRIAPPRSHCCFWEVNAPFVQGKDWGLKKFIRRDFEAFQRAAGEGGPGRGGARRGARVLQSPFCRAGAGEWLGHQSLRWGMELAASRRFSWDHHSAGGPPRVPSVRSGAAQVQPKDPLPLRTLAGCLARTAHLRPGAESLPQPQLHCTIARELHQFAFDLLIKSHKMHFSLKEHPPPPCPPHVVGYGHLDTSGSSSSSSWPQPDSFAALHSSLNELGELWFQSSELSPTGAPWPSRRPTWRGTTVSPRTATSSARTCCGTKWPSSQEAALGLGSGLLRFSCGTAAIR

SEQ ID NO: 558 - protein, 1507 aa, Artificial seqeunce
  Heterologous polypeptide
MGQKEQIHTLQKNSERMSKQLTRSSQAVDGHSYTSKVNCLLLQDGFHGCVSITGAAGRRNLSIFLFLMLCKLEFHACQWQHYHRSGEAAGTPLWRPTRNVAMMVPDRQVHYDFGLVLRFLDLKVRYLHSKIQNKNCPDVPFRELKNQRTAQGAPGIHHAASPVAANLCDPARHAQHTRIPCGAGQVRAGRGPEAGGGVLQPQRPAPEKPGCPCRRGQPRLHTVKMWRACHLFLQPQVGTPPPHTASARAPSGPPHPHESCPAGRRPARAAQTCARRQHGLPGCEEAGTARVPSLHLHLHQAALGAGRGRGWGEACAQVPPSRGHYKLIQQPISLFSITDRLHKTFSQLPSVHLCSITFQWGHPPIFCSTNDICVTANFCISVTFLKPCFLLHEASASQFKKFDGPCGERGGGRTARALWARGDSVLTPALDPQTPVRAPSLTRAAAAVQNLQNGGGSRSSATLPGRRRRRWLRRRRQPISVAPAGPPRRPNQKPNPPGGARCVIMRPTWPGTSAFTGMECTLGQVGAPSPRREEDGWRGGHSRFKADVPAPQGPCWGGQPGSAPSSAPPEQSLLDDYWAQKEKISIPRTHLCWKFEMSYTVGGPPPHVHARPRHWKTDRDYWAQKEKGSSSFLRPSCVPFRELKNVSVLEGLRQGRLGGPCSCHCPRPSQARLTPVDVAGPFLCLGDPGLFPPVKSSITEISCCTLSSEENEYLPRPEWQLQYEAGMTLGGKILFFLFLLLPLSPFSLIFLVLGVLSGHSGSRLKRSFAVTERIITGGKSTCSAPGPQSLPSTPFSTYPQWVILITELGVPGDSTRRAVRRMNTFCGASACDVSLIAMDSACEERGAAGSLISCESLYHREKQLIAMDSAIFVQGKDWGVKKFIRRDFTMPAILKLQKNCLLSLNSKMALNSEALSVVSEYEAGMTLGEKFRVGNCKHLKMTRPTEYNQKLQVNQFSESKRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGYAYKDFLWCFPFSLVFLQEIQICCHVSCLCCICCSTRICLGCLLELFLSRALRALHVLWNGFQLHCQGNTTLQQLGEASQAPSGSLIPLRLPLLWEVRGNSKMALNSLNSIDDAQLTRIAPPRSHCCFWEVNAPFVQGKDWGLKKFIRRDFEAFQRAAGEGGPGRGGARRGARVLQSPFCRAGAGEWLGHQSLRWGMELAASRRFSWDHHSAGGPPRVPSVRSGAAQVQPKDPLPLRTLAGCLARTAHLRPGAESLPQPQLHCTIARELHQFAFDLLIKSHKMHFSLKEHPPPPCPPHVVGYGHLDTSGSSSSSSWPQPDSFAALHSSLNELGELWFQSSELSPTGAPWPSRRPTWRGTTVSPRTATSSARTCCGTKWPSSQEAALGLGSGLLRFSCGTAAIR

SEQ ID NO: 559 - protein, 1507 aa, Artificial seqeunce
  Heterologous polypeptide
MGQKEQIHTLQKNSERMSKQLTRSSQAVGNTTLQQLGEASQAPSGSLIPLRLPLLWEVRGNSKMALNSLNSIDDAQLTRIAPPRSHCCFWEVNAPFVQGKDWGLKKFIRRDFEAFQRAAGEGGPGRGGARRGARVLQSPFCRAGAGEWLGHQSLRWGMELAASRRFSWDHHSAGGPPRVPSVRSGAAQVQPKDPLPLRTLAGCLARTAHLRPGAESLPQPQLHCTIARELHQFAFDLLIKSHKMHFSLKEHPPPPCPPHVVGYGHLDTSGSSSSSSWPQPDSFAALHSSLNELGELWFQSSELSPTGAPWPSRRPTWRGTTVSPRTATSSARTCCGTKWPSSQEAALGLGSGLLRFSCGTAAIRDGHSYTSKVNCLLLQDGFHGCVSITGAAGRRNLSIFLFLMLCKLEFHACQWQHYHRSGEAAGTPLWRPTRNVAMMVPDRQVHYDFGLKIQNKNCPDVLRFLDLKVRYLHSVPFRELKNQRTAQGAPGIHHAASPVAANLCDPARHAQHTRIPCGAGQVRAGRGPEAGGGVLQPQRPAPEKPGCPCRRGQPRLHTVKMWRACHLFLQPQVGTPPPHTASARAPSGPPHPHESCPAGRRPARAAQTCARRQHGLPGCEEAGTARVPSLHLHLHQAALGAGRGRGWGEACAQVPPSRGHYKLIQQPISLFSITDRLHKTFSQLPSVHLCSITFQWGHPPIFCSTNDICVTANFCISVTFLKPCFLLHEASASQFKKFDGPCGERGGGRTARALWARGDSVLTPALDPQTPVRAPSLTRAAAAVGVPGDSTRRAVRRMNTFCGASACDVSLIAMDSATEYNQKLQVNQFSESKYEAGMTLGEKFRVGNCKHLKMTRPTMPAILKLQKNCLLSLNSKMALNSEALSVVSECEERGAAGSLISCESLYHREKQLIAMDSAIFVQGKDWGVKKFIRRDFRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGYAYKDFLWCFPFSLVFLQEIQICCHVSCLCCICCSTRICLGCLLELFLSRALRALHVLWNGFQLHCQQNLQNGGGSRSSATLPGRRRRRWLRRRRQPISVAPAGPPRRPNQKPNPPGGARCVIMRPTWPGTSAFTGMECTLGQVGAPSPRREEDGWRGGHSRFKADVPAPQGPCWGGQPGSAPSSAPPEQSLLDDYWAQKEKISIPRTHLCWKFEMSYTVGGPPPHVHARPRHWKTDRDYWAQKEKGSSSFLRPSCVPFRELKNVSVLEGLRQGRLGGPCSCHCPRPSQARLTPVDVAGPFLCLGDPGLFPPVKSSITEISCCTLSSEENEYLPRPEWQLQYEAGMTLGGKILFFLFLLLPLSPFSLIFLVLGVLSGHSGSRLKRSFAVTERIITGGKSTCSAPGPQSLPSTPFSTYPQWVILITEL

SEQ ID NO: 560 - protein, 15 aa, Homo sapiens
WKFEMSYTVGGPPPH

SEQ ID NO: 561 - protein, 15 aa, Homo sapiens
VGGPPPHVHARPRHW

SEQ ID NO: 562 - protein, 14 aa, Homo sapiens
PPHVHARPRHWKTD

SEQ ID NO: 563 - protein, 15 aa, Homo sapiens
YEAGMTLGGKILFFL

SEQ ID NO: 564 - protein, 15 aa, Homo sapiens
GKILFFLFLLLPLSP

SEQ ID NO: 565 - protein, 16 aa, Homo sapiens
FFLFLLLPLSPFSLIF

SEQ ID NO: 566 - protein, 15 aa, Homo sapiens
DGHSYTSKVNCLLLQ

SEQ ID NO: 567 - protein, 15 aa, Homo sapiens
VNCLLLQDGFHGCVS

SEQ ID NO: 568 - protein, 15 aa, Homo sapiens
GFHGCVSITGAAGRR

SEQ ID NO: 569 - protein, 15 aa, Homo sapiens
TGAAGRRNLSIFLFL

SEQ ID NO: 570 - protein, 15 aa, Homo sapiens
LSIFLFLMLCKLEFH

SEQ ID NO: 571 - protein, 13 aa, Homo sapiens
LFLMLCKLEFHAC

SEQ ID NO: 572 - protein, 15 aa, Homo sapiens
TGGKSTCSAPGPQSL

SEQ ID NO: 573 - protein, 15 aa, Homo sapiens
APGPQSLPSTPFSTY

SEQ ID NO: 574 - protein, 15 aa, Homo sapiens
STPFSTYPQWVILIT

SEQ ID NO: 575 - protein, 15 aa, Homo sapiens
SRSSATLPGRRRRRW

SEQ ID NO: 576 - protein, 15 aa, Homo sapiens
DYWAQKEKGSSSFLR

SEQ ID NO: 577 - protein, 14 aa, Homo sapiens
QKEKGSSSFLRPSC

SEQ ID NO: 578 - protein, 15 aa, Homo sapiens
VPFRELKNVSVLEGL

SEQ ID NO: 579 - protein, 15 aa, Homo sapiens
VSVLEGLRQGRLGGP

SEQ ID NO: 580 - protein, 15 aa, Homo sapiens
QGRLGGPCSCHCPRP

SEQ ID NO: 581 - protein, 15 aa, Homo sapiens
SCHCPRPSQARLTPV

SEQ ID NO: 582 - protein, 15 aa, Homo sapiens
QARLTPVDVAGPFLC

SEQ ID NO: 583 - protein, 15 aa, Homo sapiens
VAGPFLCLGDPGLFP

SEQ ID NO: 584 - protein, 13 aa, Homo sapiens
GDPGLFPPVKSSI

SEQ ID NO: 585 - protein, 15 aa, Homo sapiens
GMECTLGQVGAPSPR

SEQ ID NO: 586 - protein, 15 aa, Homo sapiens
VGAPSPRREEDGWRG

SEQ ID NO: 587 - protein, 15 aa, Homo sapiens
EEDGWRGGHSRFKAD

SEQ ID NO: 588 - protein, 15 aa, Homo sapiens
HSRFKADVPAPQGPC

SEQ ID NO: 589 - protein, 15 aa, Homo sapiens
PAPQGPCWGGQPGSA

SEQ ID NO: 590 - protein, 15 aa, Homo sapiens
GGQPGSAPSSAPPEQ

SEQ ID NO: 591 - protein, 15 aa, Homo sapiens
GSAPSSAPPEQSLLD

SEQ ID NO: 592 - protein, 15 aa, Homo sapiens
GNTTLQQLGEASQAP

SEQ ID NO: 593 - protein, 15 aa, Homo sapiens
GEASQAPSGSLIPLR

SEQ ID NO: 594 - protein, 16 aa, Homo sapiens
GSLIPLRLPLLWEVRG

SEQ ID NO: 595 - protein, 15 aa, Homo sapiens
EAFQRAAGEGGPGRG

SEQ ID NO: 596 - protein, 15 aa, Homo sapiens
EGGPGRGGARRGARV

SEQ ID NO: 597 - protein, 15 aa, Homo sapiens
ARRGARVLQSPFCRA

SEQ ID NO: 598 - protein, 15 aa, Homo sapiens
QSPFCRAGAGEWLGH

SEQ ID NO: 599 - protein, 15 aa, Homo sapiens
CRAGAGEWLGHQSLR

SEQ ID NO: 600 - protein, 15 aa, Homo sapiens
DYWAQKEKISIPRTH

SEQ ID NO: 601 - protein, 13 aa, Homo sapiens
QKEKISIPRTHLC

SEQ ID NO: 602 - protein, 15 aa, Homo sapiens
VAMMVPDRQVHYDFG

SEQ ID NO: 603 - protein, 13 aa, Homo sapiens
VPDRQVHYDFGLR

SEQ ID NO: 604 - protein, 15 aa, Homo sapiens
FKKFDGPCGERGGGR

SEQ ID NO: 605 - protein, 15 aa, Homo sapiens
GERGGGRTARALWAR

SEQ ID NO: 606 - protein, 15 aa, Homo sapiens
ARALWARGDSVLTPA

SEQ ID NO: 607 - protein, 15 aa, Homo sapiens
DSVLTPALDPQTPVR

SEQ ID NO: 608 - protein, 15 aa, Homo sapiens
DPQTPVRAPSLTRAA

SEQ ID NO: 609 - protein, 14 aa, Homo sapiens
PVRAPSLTRAAAAV

SEQ ID NO: 610 - protein, 15 aa, Homo sapiens
GRRRRRWLRRRRQPI

SEQ ID NO: 611 - protein, 15 aa, Homo sapiens
GVPGDSTRRAVRRMN

SEQ ID NO: 612 - protein, 13 aa, Homo sapiens
DSTRRAVRRMNTF

SEQ ID NO: 613 - protein, 15 aa, Homo sapiens
RRRRQPISVAPAGPP

SEQ ID NO: 614 - protein, 15 aa, Homo sapiens
VAPAGPPRRPNQKPN

SEQ ID NO: 615 - protein, 15 aa, Homo sapiens
TEISCCTLSSEENEY

SEQ ID NO: 616 - protein, 16 aa, Homo sapiens
SSEENEYLPRPEWQLQ

SEQ ID NO: 617 - protein, 15 aa, Homo sapiens
RPNQKPNPPGGARCV

SEQ ID NO: 618 - protein, 15 aa, Homo sapiens
PGGARCVIMRPTWPG

SEQ ID NO: 619 - protein, 15 aa, Homo sapiens
HVVGYGHLDTSGSSS

SEQ ID NO: 620 - protein, 16 aa, Homo sapiens
YGHLDTSGSSSSSSWP

SEQ ID NO: 621 - protein, 12 aa, Homo sapiens
MRPTWPGTSAFT

SEQ ID NO: 622 - DNA, 32363 bp, Artificial sequence
  GAd20
catcatcaataatataccttattttggattgaggccaatatgataatgaggtgggcggggcgaggcggggcgggtgacgtaggacgcgcgagtagggttgggaggtgtggcggaagtgtggcatttgcaagtgggaggagctgacatgcaatcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcgccgtcatctgacgcggagggtatttaaacccgctgcgctcctaaagaggccactcttgagtgccagcgagaagagttttctcctccgctccgtttcggcgatcgaaaaatgagacatttagcctgcactccgggtcttttgtccggccgggcggcgtccgagcttttggacgctttgctcaatgaggttctgagcgatgattttccgtctactacccactttagcccacctactcttcacgaactgtacgatctggatgtactggtggatgtgaacgatcccaacgaggaggcggtttctacgttttttcccgagtctgcgcttttggctgcccaggagggatttgacctacacactccgccgctgcctattttagagtctccgctgccggagcccagtggtataccttatatgcctgaactgcttcccgaagtggtagacctgacctgccacgagccgggctttccgcccagcgacgatgagggtgagccttttgctttagactatgctgagatacctgggctcggttgcaggtcttgtgcatatcatcagagggttaccggagaccccgaggttaagtgttcgctgtgctatatgaggctgacctcttcctttatctacagtaagtttttttgtgtaggtgggctttttgggtaggtgggttttgtggcaggacaggtgtaaatgttgcttgtgttttttgtacctgcaggtccggtgtccgagccagacccggagcccgaccgcgatcccgagccggatcccgagcctcctcgcaggccaaggaaattaccttccattttgtgcaagcctaagacacctgtgaggaccagcgaggcggacagcactgactctggcacttctacctctcctcctgaaattcacccagtggttcctctgggtatacatagacctgttgctgttagagtttgcgggcgacgccctgcagtagagtgcattgaggacttgcttaacgatcccgagggacctttggacttgagcattaaacgccctaggcaataaaccccacctaagtaataaaccccacctaagtaataaactttaccgcccttggttattgagatgacgcccaatgtttgcttttgaatgacttcatgtgtataataaaagtgagtgtggtcataggtctcttgtttgtctgggcggggtttaagggtatataagtttctcggggctaaacttggttacacttgaccccaatggaggcgtgggggtgcttggaggagtttgcggacgtgcgccgtttgctggacgagagctctagcaatacctatagtatttggaggtatctgtggggctctactcaggccaagttggtcttcagaattaagcaggattacaagtgcgattttgaagagctttttagttcctgtggtgagcttttgcaatccttgaatctgggccaccaggctatcttccaggaaaaggttctctcgactttggatttttccactcccgggcgcaccgccgcttgtgtggcttttgtgtcttttgtgcaagataaatggagcggggagacccacctgagtcacggctacgtgctggatttcatggcgatggctctttggagggcttacaacaaatggaagattcagaaggaactgtacggttccgccctacgtcgtccacttctgcagcggcaggggctgatgtttcccgaccatcgccagcatcagaatctggaagacgagcgagcggagaagatcagcttgagagccggcctggaccctcctcaggaggaatgaatctcccgcaggtggttgagctgtttcccgaactgagacgggtcctgactatcagggaggatggtcagtttgtgaagaagctgaagagggatcggggtgagggagatgatgaggcggctagcaatttagcttttagtctgataactcgccaccgaccggaatgtattacctatcagcagattaaggagagttgtgccaacgagctggatcttttgggtcagaagtatagcatagaacagcttaccacttactggcttcagcccggggatgattgggaagaggcgattagggtgtatgcaaaggtggccctgcggcccgattgcaagtataagattactaagttggttaatattagaaactgctgctatatttctggaaacggggccgaagtggagatagatactgaggacagggtggctattaggtgttgcatgataaacatgtggcccgggatactggggatggatggggtgatatttatgaatgtgaggttcacgggccccaactttaatggtacggtgttcatgggcaacaccaacttgctcctgcatggtgcgagtttctatgggtttaacaacacctgtatagaggcctggaccgatgtaaaggttcgaggttgttccttttatagctgttggaaggcggtggtgtgtcgccctaaaagcaggggttctgtgaagaaatgcttgtttgaaaggtgcaccctaggtatcctttctgagggcaactccagggtgcgccataatgtggcttcgaactgcggttgcttcatgcaagtgaagggggtgagcgttatcaagcataactcggtctgtggaaactgcgaggatcgcgcctctcagatgctgacctgctttgatggcaactgtcacctgttgaagaccattcatataagcagtcaccccagaaaggcctggcccgtgtttgagcataacattctgacccgctgttccttgcatctgggggtcaggaggggtatgttcctgccttaccagtgtaactttagccacactaaaatcctgctggaacccgagtgcatgactaaggtcagcctgaatggtgtgtttgatgtgagtctgaagatttggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagcagggtgggcgtgtataaattgggggtctaaggggtctctctgtttgtcttgcaacagccgccgccatgagcgacaccggcaacagctttgatggaagcatctttagtccctatctgacagtgcgcatgcctcactgggccggagtgcgtcagaatgtgatgggttccaacgtggatggacgtcccgttctgccttcaaattcgtctactatggcctacgcgaccgtgggaggaactccgctggacgccgcgacctccgccgccgcctccgccgccgccgcgaccgcgcgcagcatggctacggacctttacagctctttggtggcgagcagcgcggcctctcgcgcgtctgctcgggatgagaaactgactgctctgctgcttaaactggaagacttgacccgggagctgggtcaactgacccagcaggtttccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattttttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgcttagctgggaggggtgcatccggggggatatgatgtgcatcttggactggatttttaggttggctatgttcccgcccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaacgtttctggcatcgctcacatcgtagttatgttccagggtgaggtcatcataggacatctttacgaatcgggggcgaagggtcccggactgggggatgatggtaccctcgggccccggggcgtagttcccctcacagatctgcatctcccaggctttcatttcagagggagggatcatatccacctgcggggcgatgaaaaagacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttgagcatatccctgacgtggatgttctccctgaccagttccgccagaaggcgctcgccgcccagcgaaagcagctcttgcaaggaagcaaaatttttcagcggtttcaggccatcggccgtgggcatgtttttcagcgtctgggtcagcagctccagcctgtcccagagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcactggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggccgagcagaccgtctcgcattccaccagccaagtgagttccgggcggtcagggtcaaaaaccaggttgcccccatgctttttgatgcgtttcttaccttggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccaacgggggggtataaaagggggtgggtgccctttcatcttcactctcttccgcatcgctgtctgcgagagccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttatcaagcttggtggcgaatgacccgtagagggcgttggagagcagcttggcgatggagcgcagggtctggtttttgtcgcggtcggctcgctccttggccgcgatgttgagttgcacgtactcgcgggccacgcacttccactcggggaacacggtggtgcgctcgtctgggatcaggcgcaccctccagccgcggttgtgcagggtgaccatgtcgacgctggtggcgacctcaccgcgcagacgctcgttggtccagcagaggcggccgcccttgcgcgagcagaaggggggtagggggtccagctggtcctcgtttggggggtccgcgtcgatggtaaagaccccggggagcaggcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggataccgaggtaggtggggtagcagcgccccccgcggatgctggcgcgcacgtagtcatagagctcgtgggagggggccagcatgttgggcccgaggttggtgcgctgggggcgctcggcgcggaagacgatctgcctgaagatggcgtgggagttggaggagatggtgggccgctggaagacgttgaagcttgcttcttgcaagcccacggagtccctgacgaaggaggcgtaggactcgcgcagcttgtgcaccagctcggcggtgacctggacgtcgagcgcacagtagtcgagggtctcgcggatgatgtcatacctatcctcccccttctttttccacagctcgcggttgaggacgaactcttcgcggtctttccagtactcttggaggggaaacccgtccgtgtccgaacggtaagagcctagcatgtagaactggttgacggcctggtaggggcagcagcccttctccacgggcagcgcgtaggcctgcgccgccttgcggagggaggtgtgggtgagggcgaaagtgtccctgaccatgactttgaggtattgatgtctgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtgtcccacgatgtagagctccaggaagcggggctggcccttgatggaggggagctttttaagttcctcgtaggtaagctcctcgggcgattccaggccgtgctcctccagggcccagtcttgcaagtgagggttggccgccaggaaggatcgccagaggtcgcgggccatgagggtctgcaggcggtcgcggaaggttctgaactgccgccccacggccattttttcgggggtgatgcagtagaaggtgagggggtctttctcccaggggtcccatctgagctctcgggcgaggtcgcgcgcggcagcgaccagagcctcgtcgccccccagtttcatgaccagcatgaagggcacgagttgcttgccaaaggctcccatccaagtgtaggtttctacatcgtaggtgacaaagaggcgctccgtgcgaggatgagagccgattgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttttactttggttgtctggccgccagcatctgtctcctggagggcgatggtggaacagaccaccacgccgcgagagccgcaggtccagatctcggcgctcggcgggcggagtttgatgacgacatcgcgcacattggagctgtccatggtctccagctcccgcggcggcaggtcagccgggagttcctggaggttcacctcgcagagacgggtcaaggcgcggacagtgttgagatggtatctgatttcaaggggcatgttggaggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctggcgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatttctgccatgaactgctcgatctcttcctcctggagatctcctcgtccggcgcgctccacggtggccgccaggtcgttggagatgcgacccatgagctgcgagaaggcgttgagtccgccctcgttccagacccggctgtagaccacgcccccctcggcgtcgcgggcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgtcgcgcacctcgcgctcgaaggccacggggggcgcttcttcctcttccacctcttcttccatgattgcttcttcttcttcctcagccgggacgggagggggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatttcgccgcggggcgggcggccgtgaggtagcgagacggcgctgactatgcatcttaacaattgctgtgtaggtacgccgccaagggacctgattgagtccagatccaccggatccgaaaacctttggaggaaagcgtctatccagtcgcagtcgcaaggtaggctgagcaccgtggcgggcgggggcgggtcgggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtaatcttgcatgagtctttccaccggcacttcttctccttcctcttcttcatctcgccggtggtttctcgcgccgcccatgcgcgtgaccccaaagcccctgagcggctgcagcagggccaggtcggcgaccacgcgctcggccaagatggcctgctgtacctgagtgagggtcctctcgaagtcatccatgtccacgaagcggtggtaggcacccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgcctgcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcagccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttggatcggccggaaccgcggctaacgtgggctgtggcagccccgtcctcaggaccccgccagccgacttctccagttacgggagcgagccccttttgtttttttattttttagatgcatcccgtgctgcggcagatgcgcccctcgccccggcccgatcagcagcagcaacagcaggcatgcagacccccctctcctctccccgccccggtcaccacggccgcggcggccgtgtccggtgcggggggcgcgctggagtcagatgagccaccgcggcggcgacctaggcagtatctggacttggaagagggcgagggactggcgcggctgggggcgagctctccagagcgccacccgcgggtgcagttgaaaagggacgcgcgtgaggcgtacctgccgcggcaaaacctgtttcgcgaccgcgggggcgaggagcccgaggagatgcgggactgcaggttccaagcggggcgcgagctgcgccgcggcttggacagacagcgcctgctgcgcgaggaggactttgagcccgacacgcagacgggcatcagccccgcgcgcgcgcacgtggccgcggccgacctggtgaccgcctacgagcagacggtgaaccaggagcgcaacttccaaaaaagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccattaactattctatgctgagcctgggcaagttctacgctcgcaagatctacaagaccccctacgtgcccatagacaaggaggtgaagatagacagcttctacatgcgcatggcgctgaaggtgctaaccctgagcgacgacctgggagtgtaccgcaacgagcgcatccacaaggccgtgagcgccagccggcggcgcgagctgagcgaccgcgaactgatgcacagtctgcagcgcgcgctgaccggcgcgggcgagggcgacagggaggtcgagtcctactttgacatgggggccgacctgcactggcagccgagccgccgcgccctggaagcggcgggggcgtacggcggccccctggcggccgatgacgaggaagaggaggactatgagctagaggagggcgagtacctggaggactgacctggctggtggtgttttggtatagatgcaagatccgaacgtggcggacccggcggtccgggcggcgctgcagagccagccgtccggcattaactcctctgacgactgggccgcggccatgggtcgcatcatggccctgaccgcgcgcaaccccgaggccttcaggcagcagcctcaggctaaccggctggcggccatcttggaagcggtagtgcccgcgcgctccaaccccacccacgagaaggtgctggccatagtcaacgcgctggcggagagcagggccatccgggcagacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggcaacctgggctcgctggtggcgctaaacgccttccttagcacccagccggccaacgtaccgcgggggcaggaggactacaccaacttcttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaggagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcttcccagcggccgttcatcaaccgcctgatggactacttgcatcgggcggcggccgtgaaccccgagtacttcaccaatgccattctgaatccccactggatgccccctccgggtttctacaacggggacttcgaggtgcctgaggtcaacgatgggttcctctgggatgacatggatgacagtgtgttctcccccaacccgctgcgcgccgcgtctctgcgattgaaggagggctctgacagggaaggaccaaggagtctggcctcctccctggctctgggggcggtgggcgccacgggcgcggcggcgcggggcagcagccccttccccagcctggcggactctctgaatagcgggcgggtgagcaggccccgcttgctaggcgaggaggagtatctgaacaactccctgctgcagcccgtgagggacaaaaacgctcagcggcagcagtttcccaacaatgggatagagagcctggtggacaagatgtccagatggaagacgtatgcgcaggagtacaaggagtgggaggaccgccagccgcggcccctgccgccccctagacagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagagaaaaataaaaactcaccaaggccatggcgacgagcgttggttttttgttcccttccttagtatgcggcgcgcggcgatgttcgaggaggggcctcccccctcttacgagagcgcgatgggaatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagtacccagaccataaacctggacaacaggtcgaactggggcggcgacctgaagactatcctgcacaccaatatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggagagcgatatcggggtcaagtttgacaccagaaacttccgtctgggctgggaccctgtgaccgggctggtcatgccgggggtctacaccaacgaggcctttcatcccgatatagtgctcctgcccggctgtggggtggacttcacccagagccggctgagcaacctgctgggcgttcgcaagcggcaacctttccaggagggtttcaagatcacctatgaggatctggaggggggcaacattcccgcgctccttgatctggacgcctacgaggagagcttgaaacccgaggagagcgctggcgacagcggcgagagtggcgaggagcaagccggcggcggcggcagcgcgtcggtagaaaacgaaagtactcccgcagtggcggcggacgctgcggaggtcgagccggaggccatgcagcaggacgcagaggagggcgcgcaggaggacatgaacaatggggagatcaggggcgacactttcgccacccggggcgaagaaaaagaggcagaggcggcggcggcgacggcggaagccgaaaccgaggcagaggcagagcccgagaccgaagttatggaagacatgaatgatggagaacgtaggggtgacacgtttgccacccggggcgaagagaaggcggcggaggcagaagccgcggctgaggaggcggctgcggctgcggccaaggctgaggctgcggctgaggctaaggtcgaagccgatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcattgagggcagcacctttacccaataccgcagctggtacctggcttacaactacggcgacccggtcaagggggtgcgctcgtggaccctgctctgcacgccggacgtcacctgcggctccgagcagatgtactggtcgctgccaaacatgatgcaagacccggtgaccttccgttccacgcggcaggttagcaactttccggtggtgggcgccgaactgctgccagtacactccaagagtttttacaacgagcaggccgtctactcccagctgatccgccaggccacctctctgacccacgtgttcaatcgctttcccgagaaccagattttggcgcgcccgccggcccccaccatcaccaccgtcagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatccacccacacgctccaaaatcatgtccgtactcatctcgcccagcaacaacaccggctgggggctgcgcgcacccagcaagatgtttggaggggcaaggaagcgctccgaccagcaccccgtgcgcgtgcgcggccactaccgcgcgccctggggtgcgcacaagcgcgggcgcacagggcgcaccactgtggatgatgtcattgactccgtagtggagcaggcgcgccactacacacccggcgcgccgaccgcctccgccgtgtccaccgtggaccaggcgatcgaaagcgtggtacagggggcgcggcactatgccaaccttaaaagtcgccgccgccgcgtggcgcgccgccatcgccggagaccccgggctactgccgccgcgcgccttaccaaggctctgctcaagcgcgccaggcgaactggccaccgggccgccatgagggccgcacggcgggctgccgctgccgcgagcgccgtggccccgcgggcacgaaggcgcgcggccgctgccgccgccgccgccatttccagcttggcctcgacgcggcgcggtaacatatactgggtgcgcgactcggtgagcggcacacgtgtgcccgtgcgctttcgccccccacggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggaggattacaagccccgcaagctaaagcgggtcaaaaagaaaaagaaagatgatgacgttgacgaggcggtggagtttgtccgccgcatggcgcccaggcgccctgtgcagtggaagggtcggcgcgtgcagcgagtcctgcgccccggcaccgcggtggtctttacgcccggcgagcgttccacgcgcactttcaagcgggtgtacgatgaggtgtacggcgacgaggatctgttggagcaggccaaccatcgatttggggagtttgcatatgggaaacggcctcgcgagagtctaaaagaggacctgctggcgctaccgctggacgagggcaatcccaccccgagtctgaagccggtgaccctgcaacaggtgctgcctttgagcgcgcccagcgagcagaagcgagggttaaagcgcgagggcggggacctggcacccaccgtgcagttgatggtgcccaagcggcagaagctggaggacgtgctggagaaaatgaaagtagagcccgggatccagcccgagatcaaggtccgccctatcaagcaggtggcgcccggcgtgggagtccagaccgtggacgttaggattcccacggaggagatggaaacccaaaccgccactccctcttcggcagcaagcgccaccaccggcgccgcttcggtagaggtgcagacggacccctggctacccgccgccactatcgccgtcgccgccgccccccgttcgcgcggacgcaagagaaattatccagcggccagcgcgcttatgccccagtatgcgctgcatccatccatcgcgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgccttcgcttcccggtgccgggataccgaggaagaactcaccgccgcaggggcatggcgggcagcggtctccgcggcggccgtcgccatcgccggcgcgcaaagagcaggcgcatgcgcggcggtgtgttgcccctgctggtcccgctactcgccgcggcgatcggcgccgtgcccgggatcgcctccgtggccctgcaggcgtcccagaaacattgactcttgcaaccttgcaagcttgcatttttggaggaaaaaataaaaaagtctagactctcacgctcgcttggtcctgtgactattttgtagaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggtcagatgctgagagacaagttgaaagagcagaacttccaggagaaggtggcgcagggcctggcctctggcatcagcggggtggtggacatagctaaccaggccgtgcagaaaaagataaacagtcatctggacccccgccctcaggtggaggaaacgcctccagccatggagacggtgtctcccgagggcaaaggcgaaaagcgcccgcggcccgacagggaagagaccctggtgtcacacaccgaggagccgccctcttacgaggaggcagtcaaggccggcctgcccaccactcgccccatagctcccatggccaccggtgtggtgggtcacaggcaacacacccccgcaacactagatctgcccccgccgtccgagccgactcgccagccaaaggcggtgacggtgtccgctccctccacttccgccgccaacagagtgcctctgcgccgcgctgcgagcggcccccgggcctcgcgagtcagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcttactcttacaaggcgcggttcacgctggccgtgggcgacaaccgcgtgctggacatggcctccacttactttgacatccggggggtgctggacaggggccccacttttaagccctactcgggcactgcctacaaccccctggcccccaagggcgcccccaattcttgtgagtgggaacaagaggaaaatcaggtggtcgctgcagatgatgaacttgaagatgaagaagcgcaagcacaagaggaagcccctgtgaaaaaaattcatgtatatgctcaggcgcctctttctggcgaaaagatttccaaggatggtatccaaataggtactgaagtcgtaggagatacatctaaggacacttttgcagataaaacattccaacccgaacctcagataggcgagtctcagtggaacgaggctgatgccacagcagcaggaggtagagttttgaaaaagactacccctatgagaccttgctatggatcctatgccaggcctaccaatgccaacgggggtcaaggaattatggttgccaatgaacaaggagtgttggagtctaaagtagaaatgcaatttttctctaacaccacaacccttaatgcgcgggatggaaccggcaatcccgaaccaaaggtggtgttgtacagcgaagatgtccacttggaatctcccgatactcatctgtcttacaagcccaaaaaggatgatgttaatgccaaaatcatgttgggtcagcaagccatgcccaacagacccaacctcattggatttagagataatttcattgggcttatgttttacaacagcaccggtaacatgggagtgctggcgggtcaggcctctcagttgaatgctgtggtggacttgcaggatagaaacacagaactgtcatatcagcttctgcttgattcaattggggatagaaccagatacttctccatgtggaaccaggcagtggatagctatgatccagatgtcagaattattgaaaaccatgggactgaggatgaactgcccaactactgcttccctttgggcggcataggagttactgatacttatcaagggataaaaaataccaatggcaatggtcagtggaccaaagatgatcagttcgcggaccgcaacgaaataggggtgggaaacaacttcgccatggagatcaacatccaggccaacctttggagaaacttcctctatgcaaacgtggggctctacctgccagacaagctcaagtacaaccccaccaacgtggacatctctgacaaccccaacacctatgactacatgaacaagcgggtggtggcccctggcctggtggactgctttgtcaatgtgggagccaggtggtccctggactacatggacaacgtcaaccccttcaaccaccaccgcaatgcgggtctgcgctaccgctccatgatcctgggcaacgggcgctatgtgccctttcacatccaggtaccccagaagttctttgccatcaagaacctcctgctcctgcccggctcctacacctacgagtggaacttcaggaaggatgtgaacatggtcctacagagctctctgggcaatgaccttagggtggatggggccagcatcaagtttgacagcatcaccctctatgctacatttttccccatggcccacaacaccgcctccacgcttgaggccatgctgagaaacgacaccaacgaccagtcctttaatgactacctctctggggccaacatgctctacccaatcccagccaaggccaccaacgtgcccatctccatcccctctcgcaactgggccgcctttagaggctgggcctttacccgccttaagaccaaggagaccccctccctgggctcgggttttgatccctactttgtttactcgggatccatcccctacctggatggcaccttctacctcaaccacactttcaagaagatatccatcatgtatgactcctccgtcagctggccgggcaacgaccgcttgctcacccccaatgagttcgaggtcaagcgcgccgtggacggcgagggctacaacgtggcccagtgcaacatgaccaaggactggttcctggtgcagatgctggccaactacaacataggctaccagggcttttacatcccagagagctacaaggacaggatgtactccttcttcagaaatttccaacccatgagccgacaggtggtggacgagaccaattacaaggactatcaagccattggcatcacccaccagcacaacaactcgggtttcgtgggctacctggcgcccaccatgcgcgagggtcaggcctaccccgccaacttcccctaccccttgataggcaagaccgcggtcgacagcgtcacccagaaaaagttcctctgcgaccgcaccctctggcgcatccccttctctagcaacttcatgtccatgggtgcgctcacggacctgggccaaaacctgctttatgccaactctgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgtacgcccttctcagccggcaacgccaccacctaaggagacagcgccgccgccgcctgcatgacgggttccaccgagcaagagctcagggccattgccagagacctgggatgcggaccctattttttgggcacctatgacaaacgcttcccgggctttatctcccgagacaagctcgcctgcgccattgtcaacacggccgcgcgcgagaccgggggcgtgcactggctggcctttggctgggacccgcgctccaaaacttgctacctctttgacccctttggcttctccgatcagcgcctcaggcagatttatgagtttgagtacgaggggctgctgcgccgcagcgcgctcgcctcctcgcccgaccgctgcatcacccttgagaagtccaccgaaaccgtgcaggggccccactcggccgcctgcggtctcttctgttgcatgtttttgcacgcctttgtgcactggcctcagagtcccatggattgcaaccccaccatgaacttgctaaagggagtgcccaacgccatgctccagagcccccaggtccagcccaccctgcgccgcaaccaggaacagctttaccgcttcctggagcgccactccccctacttccgcagccacagcgcgcgcatccggggggccacctctttttgccacttgcaagaaaacatgcaagacggaaaatgatgtacagcatgcttttaataaatgtaaagactgtgcactttaattatacacgggctctttctggttatttattcaacaccgccgtcgccatttagaaatcgaaagggttctgccgtgcgtcgccgtgcgccacgggcagagacacgttgcgatactggaagcggctcgcccacttgaactcgggcaccaccatgcggggcagtggttcctcggggaagttctcgctccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattattcacgctggccagcaggctctcgtcgctgatcatgtcgctgtccagatcctccgcgttgctcagggcgaatggggtcatcttgcagacctgcctgcccaggaaaggcgggagcccaggcttgccgttgcagtcgcagcgcaggggcattagcaggtgcccacggcccgactgcgcctgcgggtacaacgcgcgcatgaaggcttcgatctgcctaaaagccacctgggtcttggctccctccgaaaagaacatcccacaggacttgctggagaactggttcgcgggacagctggcatcgtgcaggcagcagcgcgcgtcagtgttggcaatctgcaccacgttgcgaccccaccggtttttcactatcttggccttggaagcctgctcctttagcgcgcgctggccgttctcgctggtcacatccatctctatcacctgttccttgttgatcatgtttgtcccgtgcagacactttaggtcgccctccgtctgggtgcagcggtgctcccacagcgcgcaaccggtgggctcccaattcttgtgggtcacccccgcgtaggcctgcaggtaggcctgcaggaagcgccccatcatggtcataaaggtcttctggctcgtaaaggtcagctgcaggccgcgatgctcttcgttcagccaggtcttgcagatggcggccagcgcctcggtctgctcgggcagcatcttaaaatttgtcttcaggtcgttatccacgtggtacttgtccatcatggcacgcgccgcctccatgcccttctcccaggcggacaccatgggcaggcttagggggtttatcacttccagcggcgaggacaccgtactttcgatttcttcttcctccccctcttcccggcgcgcgcccccgctgttgcgcgctcttaccgcctgcaccaaggggtcgtcttcaggcaagcgccgcaccgagcgcttgccgcccttgacctgcttgatcagtaccggcgggttgctgaagcccaccatggtcagcgccgcctgctcttcttcgtcttcgctgtctaccactatttctggggaggggcttctccgctctgcggcaaaggcggcggatcgcttcttttttttcttgggagccgccgcgatggagtccgccacggcgaccgaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgcgctggtcttggtcccgactggccattgtatcctcctcctcctaggcagagagacataaggagtctatcatgcaagtcgagaaggaggagagcttaaccaccccctcagagaccgccgatgcgcccgccgtcgccgtcgcccccgctaccgccgacgcgcccgccacaccgagcgacacccccacggacccccccgccgacgcacccctgttcgaggaagcggccgtggagcaggacccgggctttgtctcggcagaggaggatttgcaagaggaggagaataaggaggagaagccctcagtgccaaaagatcataaagagcaagacgagcacgacgcagacgcacaccagggtgaagtcgggcggggggacggagggcatggcggcgccgactacctagacgaaggaaacgacgtgctcttgaagcacctgcatcgtcagtgcgccatcgtctgcgacgctctgcaggagcgcagcgaggtgcccctcagcgtggcggaggtcagccgcgcctacgagctcagcctcttttccccccgggtgcccccccgccgccgcgaaaacggcacatgcgagcccaacccgcgcctcaacttctaccccgcctttgtggtgcccgaggtcctggccacctatcacatcttctttcaaaattgcaagatccccatctcgtgccgcgccaaccgtagccgcgccgataagatgctggccctgcgccagggcgaccacatacctgatatcgccgctttggaagatgtgccaaagatcttcgagggtctggggcgcaacgagaagcgggcagcaaactctctgcaacaggaaaacagcgaaaatgagagtcacactggagcgctggtggagctggagggcgacaacgcccgcctggcggtgctcaagcgcagcatcgaggtcacccactttgcctaccccgcgctcaacctgccccccaaagtcatgaacgcggtcatggacgggctgatcatgcgccgcggccggcccctcgctccagatgcaaacttgcatgaggagaccgaggacggtcagcccgtggtcagcgacgagcagctgacgcgctggctggagagcgcggaccccgccgaactggaggagcggcgcaagatgatgatggccgcggtgctggtcaccgtagagctggagtgtctgcagcgcttcttcggtgaccccgagatgcagagaaaggtcgaggagaccctacactacaccttccgccagggctacgtgcgccaggcttgcaagatctccaacgtggagctcagcaacctggtgtcctacctgggcatcttgcatgaaaaccgccttgggcagagcgtgctacactccaccctgcgcggggaggcgcgccgcgactacgtgcgcgactgcgtttacctcttcctctgctacacctggcagacggccatgggggtctggcagcagtgcctggaggagcgcaacctcaaggagctggagaagcttctgcagcgcgcgctcaaagacctctggacgggcttcaacgagcgctcggtggccgccgcgctagccgacctcatcttccccgagcgcctgctcaaaaccctccagcaggggctgcccgacttcaccagccaaagcatgttgcaaaattttaggaactttatcctggagcgttctggcatcctacccgccacctgctgcgccctgcccagcgactttgtccccctcgtgtaccgcgagtgccccccgccgctgtggggccactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgaaatcaggttttacgaggaccaatcccgcccgcccaaggcggagctgaccgcctgcgtcatcacccagggcgagatcctaggccaattgcaagccatccaaaaagcccgccaagagtttttgctgaagaggggtcggggggtgtatctggacccccagtcgggtgaggagctcaacccggttcccccgctgccaccgccgcgggaccttgcttcccaggataagcatcgccatggctcccagaaagaagcagcagcggccgccgctgccgccgccccacatgctggaggaagaggaggaatactgggacagtcaggcagaggaggtttcggacgaggaggagccggagacggagatggaagagtgggaggaggacagcttagacgaggaggcttccgaagccgaagaggcaggcgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagaggcggccagcggcggcggcgcccgtttcggtgcctaggaagacccagggcaagacttcagccaagaaactcgcggcgaccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgacatccgcacccatcgaaaccaaatattggtgaagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcagagctcgtcctcggcgccgcgctcgggcggcatcgggactctccagttcgtgcaggagtttgtgccctcggtctacttcaaccccttctcgggctctcccggtcgctacccggaccagtttatcccgaactttgacgccgcgagggactcggtggacggctacgactgaatgtcgggtggacccggtgcagagcaacttcgcctgaagcaccttgaccactgccgccgccctcagtgctttgcccgctgtcagaccggtgagttccagtacttttccctgcccgactcgcacccggacggcccggcgcacggggtgcgctttttcatcccgagtcaggtccgctctaccctaatcagggagttcaccgcccgtcccctactggcggagttggaaaaggggccttctatcctaaccattgcctgcatttgctctaaccctggattacaccaagatctttgctgtcatttgtgtgctgagtataataaaggctgagatcagaatctactcgggctcctgtcgccatcctgtcaacgccaccgtccaagcccggcccgatcagcccgaggtgaacctcacctgtggtctgcaccggcgcctgaggaaatacctagcttggtactacaacagcactccctttgtggtttacaacagctttgaccaggacggggtctcactgagggataacctctcgaacctgagctactccatcaggaagaacaacaccctcgagctacttcctccttacctgcccgggacttaccagtgtgtcaccggcccctgcacccacacccacctgttgatcgtaaacgactctcttccgagaacagacctcaataactcctctccgcagttccccagaacaggaggtgagctcaggaaaccccgggtaaagaagggtggacaagagttaacacttgtggggtttctggtatatgtgacgctggtggtggctcttttgattaaggcttttccttccatgtctgaactatccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccaccgcaggtagaataatgctgcttaccctctttgtcctggcgctggctgccagctgccaagccttttccgaggctgacttcatagagccccagtgcaatatcacttataaatctgaacgtgccatctgtactattctaatcaaatgtgttactcaacacgataaggtgactgttaaatacaaagatcaattaaaaaaagacgcactttacagcagctggcaaccaggagatgatcaaaaatacaatgtaaccgtcttccagggcaaactctccaaaacttacaattacaatttcccatttgagcagatgtgtgactttgtcatgtacatggaaaagcagtacaagctgtggcctccaactccccagggctgtgtggaaaatccaggctctttctgtatgatctctctctgtgtaactgtgctggcactaatactcacgcttctgtatctcagatttaaatcaaggcaaagcttcattgatgaaaagaaaatgccataatcgctcaacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtttcttactgcactaacaaaaacagccacaagcccagagccatctgcgatgggcaaaatctaaccttgattgatgttcaattgctggatgcgggctactattatgggcagctgggtacaatgattaattactggagaccccacagagattacatgcttcacgtagtaaagggtcccattagcagcccaaccaccacctctaccacacccactaccaccactactcccaccaccagcactgccgcccagcctcctcatagcagaacaaccacttttatcaattccaagtcccactccccccacattgccggcgggccctccgcctcagactccgagaccaccgagatctgcttctgcaaatgctctgacgccattgcccaggatttggaagatcacgaggaagatgagcatgactacgcagatgcatgccaggcatcagaggcagaagcgctaccggtggccctaaaacagtatgcagactcccacaccacccccaaccttcctccaccttcccagaagccaagtttcctgggggaaaatgaaactctgcctctttccatactagctctgacatctgttgctattttggccgctctgctggtgcttctatgctctatatgctacctgatctgctgcagaaagaaaaaatctcacggccatgctcaccagcccctcatgcacttcccttaccctccagagctgggcgaccacaaactttaagtctgcagtagctatctgcccatcccttgtcagtcgacagcgatgagccccactaatctaacagcctctggacttacaacattgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggcccttcgtcatcacacctgaagataatgatgatgatgacaccacctccaggctgcagagcctaaagcagctactcttctcttttacagcatggtaaattgaatcatgccccgcattttcatctacttgcttctccttccactttttctgggctcctctacattggccactgtgtcccacatcgaggtagactgcctcacgcccttcacagtctacctgcttttcggctttgtcatctgcacctttgtctgcagcgttatcactgtagtgatctgcttcatacagtgcatcgactacatctgtgtgcgggtggcctactttagacaccacccccagtatcgcaacagggacatagcggctctcctaagacttgtttaaatcatggccaaattacctgtgattggtcttctgattatctgctgcgtcctagccgcgattgggactcaacctaataccaccaccagcgctcccagaaagagacatgtatcctgcagcttcaagcgtccctggaatataccccaatgctttactgatgaacctgaaatctctttggcttggtacttcagcgtcaccgcccttctcatcttctgcagtacggttattgctcttgccatctacccttcccttaacctgggctggaatgctgtcaactctatggaatatcccaccttcccagaaccagacctgccagacctggttgttctaaacgcgtttcctcctcctccagttcaaaatcagtttcgccctccgtcccctacgcccactgaggtcagctactttaatctaacaggcggagatgactgaaaacctagacctagaaatggacggtctctgcagcgagcaacgcacactagagaggcgccggcaaaaagcagagctcgagcgtcttaaacaagagctccaagacgccgtggccatacaccagtgcaaaaaagggctcttctgtctggtaaaacaggccacgctcacctatgaaaaaacaggtgacacccaccgcctaggatacaagctgcccacacagcgccaaaagtttgcccttatgataggtgaacaacccatcaccgtcacccagcactccgtggagacagaaggctgcattcatgctccctgcaggggcgctgactgcctctacaccttgatcaaaaccctctgcggtctcagagaccttatccctttcaattgatcataactgtaatcaataaaaaatcacttacttgaaatctgatagcaagactctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttacagatgaaacgcgcgagatcgtctgacgagaccttcaaccccgtgtacccctacgataccgagatcgctccgacttctgtccctttccttacccctccctttgtatcatccgcaggaatgcaagaaaatccagctggggtgctgtccctg

SEQ ID NO: 623 - protein, 1507 aa, Artificial seqeunce
  Heterologous polypeptide
MGQKEQIHTLQKNSERMSKQLTRSSQAVGVPGDSTRRAVRRMNTFYEAGMTLGEKFRVGNCKHLKMTRPNSKMALNSEALSVVSECGASACDVSLIAMDSAFVQGKDWGVKKFIRRDFYAYKDFLWCFPFSLVFLQEIQICCHVSCLCCICCSTRICLGCLLELFLSRALRALHVLWNGFQLHCQTEYNQKLQVNQFSESKSLYHREKQLIAMDSAICEERGAAGSLISCETMPAILKLQKNCLLSLRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGGNTTLQQLGEASQAPSGSLIPLRLPLLWEVRGQPDSFAALHSSLNELGEIARELHQFAFDLLIKSHFVQGKDWGLKKFIRRDFWGMELAASRRFSWDHHSAGGPPRVPSVRSGAAQVQPKDPLPLRTLAGCLARTAHLRPGAESLPQPQLHCTLWFQSSELSPTGAPWPSRRPTWRGTTVSPRTATSSARTCCGTKWPSSQEAALGLGSGLLRFSCGTAAIRKMHFSLKEHPPPPCPPEAFQRAAGEGGPGRGGARRGARVLQSPFCRAGAGEWLGHQSLRHVVGYGHLDTSGSSSSSSWPNSKMALNSLNSIDDAQLTRIAPPRSHCCFWEVNAPDGHSYTSKVNCLLLQDGFHGCVSITGAAGRRNLSIFLFLMLCKLEFHACKIQNKNCPDFKKFDGPCGERGGGRTARALWARGDSVLTPALDPQTPVRAPSLTRAAAAVHYKLIQQPISLFSITDRLHKTFSQLPSVHLCSITFQWGHPPIFCSTNDICVTANFCISVTFLKPCFLLHEASASQCHLFLQPQVGTPPPHTASARAPSGPPHPHESCPAGRRPARAAQTCARRQHGLPGCEEAGTARVPSLHLHLHQAALGAGRGRGWGEACAQVPPSRGVLRFLDLKVRYLHSQWQHYHRSGEAAGTPLWRPTRNVPFRELKNQRTAQGAPGIHHAASPVAANLCDPARHAQHTRIPCGAGQVRAGRGPEAGGGVLQPQRPAPEKPGCPCRRGQPRLHTVKMWRAVAMMVPDRQVHYDFGLQNLQNGGGSRSSATLPGRRRRRWLRRRRQPISVAPAGPPRRPNQKPNPPGGARCVIMRPTWPGTSAFTKRSFAVTERIILVLGVLSGHSGSRLYEAGMTLGGKILFFLFLLLPLSPFSLIFTEISCCTLSSEENEYLPRPEWQLQVPFRELKNVSVLEGLRQGRLGGPCSCHCPRPSQARLTPVDVAGPFLCLGDPGLFPPVKSSITGGKSTCSAPGPQSLPSTPFSTYPQWVILITELGMECTLGQVGAPSPRREEDGWRGGHSRFKADVPAPQGPCWGGQPGSAPSSAPPEQSLLDDYWAQKEKGSSSFLRPSCDYWAQKEKISIPRTHLCWKFEMSYTVGGPPPHVHARPRHWKTDR

SEQ ID NO: 624 - protein, 1507 aa, Artificial seqeunce
  Heterologous polypeptide
MGQKEQIHTLQKNSERMSKQLTRSSQAVGNTTLQQLGEASQAPSGSLIPLRLPLLWEVRGQPDSFAALHSSLNELGEIARELHQFAFDLLIKSHFVQGKDWGLKKFIRRDFWGMELAASRRFSWDHHSAGGPPRVPSVRSGAAQVQPKDPLPLRTLAGCLARTAHLRPGAESLPQPQLHCTLWFQSSELSPTGAPWPSRRPTWRGTTVSPRTATSSARTCCGTKWPSSQEAALGLGSGLLRFSCGTAAIRKMHFSLKEHPPPPCPPEAFQRAAGEGGPGRGGARRGARVLQSPFCRAGAGEWLGHQSLRHVVGYGHLDTSGSSSSSSWPNSKMALNSLNSIDDAQLTRIAPPRSHCCFWEVNAPGVPGDSTRRAVRRMNTFYEAGMTLGEKFRVGNCKHLKMTRPNSKMALNSEALSVVSECGASACDVSLIAMDSAFVQGKDWGVKKFIRRDFYAYKDFLWCFPFSLVFLQEIQICCHVSCLCCICCSTRICLGCLLELFLSRALRALHVLWNGFQLHCQTEYNQKLQVNQFSESKSLYHREKQLIAMDSAICEERGAAGSLISCETMPAILKLQKNCLLSLRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGDGHSYTSKVNCLLLQDGFHGCVSITGAAGRRNLSIFLFLMLCKLEFHACKIQNKNCPDFKKFDGPCGERGGGRTARALWARGDSVLTPALDPQTPVRAPSLTRAAAAVHYKLIQQPISLFSITDRLHKTFSQLPSVHLCSITFQWGHPPIFCSTNDICVTANFCISVTFLKPCFLLHEASASQCHLFLQPQVGTPPPHTASARAPSGPPHPHESCPAGRRPARAAQTCARRQHGLPGCEEAGTARVPSLHLHLHQAALGAGRGRGWGEACAQVPPSRGVLRFLDLKVRYLHSQWQHYHRSGEAAGTPLWRPTRNVPFRELKNQRTAQGAPGIHHAASPVAANLCDPARHAQHTRIPCGAGQVRAGRGPEAGGGVLQPQRPAPEKPGCPCRRGQPRLHTVKMWRAVAMMVPDRQVHYDFGLQNLQNGGGSRSSATLPGRRRRRWLRRRRQPISVAPAGPPRRPNQKPNPPGGARCVIMRPTWPGTSAFTKRSFAVTERIITEISCCTLSSEENEYLPRPEWQLQYEAGMTLGGKILFFLFLLLPLSPFSLIFDYWAQKEKGSSSFLRPSCVPFRELKNVSVLEGLRQGRLGGPCSCHCPRPSQARLTPVDVAGPFLCLGDPGLFPPVKSSITGGKSTCSAPGPQSLPSTPFSTYPQWVILITELGMECTLGQVGAPSPRREEDGWRGGHSRFKADVPAPQGPCWGGQPGSAPSSAPPEQSLLDDYWAQKEKISIPRTHLCLVLGVLSGHSGSRLWKFEMSYTVGGPPPHVHARPRHWKTDR

SEQ ID NO: 625 - protein, 1507 aa, Artificial seqeunce
  Heterologous polypeptide
MGQKEQIHTLQKNSERMSKQLTRSSQAVGNTTLQQLGEASQAPSGSLIPLRLPLLWEVRGNSKMALNSLNSIDDAQLTRIAPPRSHCCFWEVNAPFVQGKDWGLKKFIRRDFEAFQRAAGEGGPGRGGARRGARVLQSPFCRAGAGEWLGHQSLRWGMELAASRRFSWDHHSAGGPPRVPSVRSGAAQVQPKDPLPLRTLAGCLARTAHLRPGAESLPQPQLHCTIARELHQFAFDLLIKSHKMHFSLKEHPPPPCPPHVVGYGHLDTSGSSSSSSWPQPDSFAALHSSLNELGELWFQSSELSPTGAPWPSRRPTWRGTTVSPRTATSSARTCCGTKWPSSQEAALGLGSGLLRFSCGTAAIRQNLQNGGGSRSSATLPGRRRRRWLRRRRQPISVAPAGPPRRPNQKPNPPGGARCVIMRPTWPGTSAFTGMECTLGQVGAPSPRREEDGWRGGHSRFKADVPAPQGPCWGGQPGSAPSSAPPEQSLLDDYWAQKEKISIPRTHLCWKFEMSYTVGGPPPHVHARPRHWKTDRDYWAQKEKGSSSFLRPSCVPFRELKNVSVLEGLRQGRLGGPCSCHCPRPSQARLTPVDVAGPFLCLGDPGLFPPVKSSITEISCCTLSSEENEYLPRPEWQLQYEAGMTLGGKILFFLFLLLPLSPFSLIFLVLGVLSGHSGSRLKRSFAVTERIITGGKSTCSAPGPQSLPSTPFSTYPQWVILITELDGHSYTSKVNCLLLQDGFHGCVSITGAAGRRNLSIFLFLMLCKLEFHACQWQHYHRSGEAAGTPLWRPTRNVAMMVPDRQVHYDFGLVLRFLDLKVRYLHSKIQNKNCPDVPFRELKNQRTAQGAPGIHHAASPVAANLCDPARHAQHTRIPCGAGQVRAGRGPEAGGGVLQPQRPAPEKPGCPCRRGQPRLHTVKMWRACHLFLQPQVGTPPPHTASARAPSGPPHPHESCPAGRRPARAAQTCARRQHGLPGCEEAGTARVPSLHLHLHQAALGAGRGRGWGEACAQVPPSRGHYKLIQQPISLFSITDRLHKTFSQLPSVHLCSITFQWGHPPIFCSTNDICVTANFCISVTFLKPCFLLHEASASQFKKFDGPCGERGGGRTARALWARGDSVLTPALDPQTPVRAPSLTRAAAAVGVPGDSTRRAVRRMNTFCGASACDVSLIAMDSACEERGAAGSLISCESLYHREKQLIAMDSAIFVQGKDWGVKKFIRRDFTMPAILKLQKNCLLSLNSKMALNSEALSVVSEYEAGMTLGEKFRVGNCKHLKMTRPTEYNQKLQVNQFSESKRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGYAYKDFLWCFPFSLVFLQEIQICCHVSCLCCICCSTRICLGCLLELFLSRALRALHVLWNGFQLHCQ

SEQ ID NO: 626 - protein, 1507 aa, Artificial seqeunce
  Heterologous polypeptide
MGQKEQIHTLQKNSERMSKQLTRSSQAVDGHSYTSKVNCLLLQDGFHGCVSITGAAGRRNLSIFLFLMLCKLEFHACVPFRELKNQRTAQGAPGIHHAASPVAANLCDPARHAQHTRIPCGAGQVRAGRGPEAGGGVLQPQRPAPEKPGCPCRRGQPRLHTVKMWRAQWQHYHRSGEAAGTPLWRPTRNKIQNKNCPDVAMMVPDRQVHYDFGLVLRFLDLKVRYLHSCHLFLQPQVGTPPPHTASARAPSGPPHPHESCPAGRRPARAAQTCARRQHGLPGCEEAGTARVPSLHLHLHQAALGAGRGRGWGEACAQVPPSRGHYKLIQQPISLFSITDRLHKTFSQLPSVHLCSITFQWGHPPIFCSTNDICVTANFCISVTFLKPCFLLHEASASQFKKFDGPCGERGGGRTARALWARGDSVLTPALDPQTPVRAPSLTRAAAAVQNLQNGGGSRSSATLPGRRRRRWLRRRRQPISVAPAGPPRRPNQKPNPPGGARCVIMRPTWPGTSAFTGMECTLGQVGAPSPRREEDGWRGGHSRFKADVPAPQGPCWGGQPGSAPSSAPPEQSLLDDYWAQKEKISIPRTHLCWKFEMSYTVGGPPPHVHARPRHWKTDRDYWAQKEKGSSSFLRPSCVPFRELKNVSVLEGLRQGRLGGPCSCHCPRPSQARLTPVDVAGPFLCLGDPGLFPPVKSSITEISCCTLSSEENEYLPRPEWQLQYEAGMTLGGKILFFLFLLLPLSPFSLIFLVLGVLSGHSGSRLKRSFAVTERIITGGKSTCSAPGPQSLPSTPFSTYPQWVILITELGNTTLQQLGEASQAPSGSLIPLRLPLLWEVRGNSKMALNSLNSIDDAQLTRIAPPRSHCCFWEVNAPFVQGKDWGLKKFIRRDFEAFQRAAGEGGPGRGGARRGARVLQSPFCRAGAGEWLGHQSLRWGMELAASRRFSWDHHSAGGPPRVPSVRSGAAQVQPKDPLPLRTLAGCLARTAHLRPGAESLPQPQLHCTIARELHQFAFDLLIKSHKMHFSLKEHPPPPCPPHVVGYGHLDTSGSSSSSSWPQPDSFAALHSSLNELGELWFQSSELSPTGAPWPSRRPTWRGTTVSPRTATSSARTCCGTKWPSSQEAALGLGSGLLRFSCGTAAIRGVPGDSTRRAVRRMNTFCGASACDVSLIAMDSACEERGAAGSLISCESLYHREKQLIAMDSAIFVQGKDWGVKKFIRRDFYEAGMTLGEKFRVGNCKHLKMTRPTMPAILKLQKNCLLSLNSKMALNSEALSVVSETEYNQKLQVNQFSESKRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGYAYKDFLWCFPFSLVFLQEIQICCHVSCLCCICCSTRICLGCLLELFLSRALRALHVLWNGFQLHCQ

SEQ ID NO: 627 - protein, 7 aa, Artificial seqeunce
  protein tag
SHHHHHH

SEQ ID NO: 628 - DNA, 845 bp, Artificial sequence
  Promoter
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtga

SEQ ID NO: 629 - DNA, 227 bp, Artificial sequence
  polyA site
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcc

SEQ ID NO: 630 - DNA, 149 bp, Artificial sequence
  Promoter
gatcactaattccaaacccacccgctttttatagtaagtttttcacccataaataataaatacaataattaatttctcgtaaaagtagaaaatatattctaatttattgcacggtaaggaagtagaatcataaagaacagtgacggatc

SEQ ID NO: 631 - protein, 15 aa, Homo sapiens
VPFRELKNQRTAQGA

SEQ ID NO: 632 - protein, 15 aa, Homo sapiens
QRTAQGAPGIHHAAS

SEQ ID NO: 633 - protein, 15 aa, Homo sapiens
GIHHAASPVAANLCD

SEQ ID NO: 634 - protein, 15 aa, Homo sapiens
VAANLCDPARHAQHT

SEQ ID NO: 635 - protein, 15 aa, Homo sapiens
ARHAQHTRIPCGAGQ

SEQ ID NO: 636 - protein, 15 aa, Homo sapiens
IPCGAGQVRAGRGPE

SEQ ID NO: 637 - protein, 15 aa, Homo sapiens
RAGRGPEAGGGVLQP

SEQ ID NO: 638 - protein, 15 aa, Homo sapiens
GGGVLQPQRPAPEKP

SEQ ID NO: 639 - protein, 15 aa, Homo sapiens
RPAPEKPGCPCRRGQ

SEQ ID NO: 640 - protein, 15 aa, Homo sapiens
CPCRRGQPRLHTVKM

SEQ ID NO: 641 - protein, 14 aa, Homo sapiens
RGQPRLHTVKMWRA

SEQ ID NO: 642 - protein, 15 aa, Homo sapiens
CHLFLQPQVGTPPPH

SEQ ID NO: 643 - protein, 15 aa, Homo sapiens
VGTPPPHTASARAPS

SEQ ID NO: 644 - protein, 15 aa, Homo sapiens
ASARAPSGPPHPHES

SEQ ID NO: 645 - protein, 15 aa, Homo sapiens
PPHPHESCPAGRRPA

SEQ ID NO: 646 - protein, 15 aa, Homo sapiens
PAGRRPARAAQTCAR

SEQ ID NO: 647 - protein, 15 aa, Homo sapiens
AAQTCARRQHGLPGC

SEQ ID NO: 648 - protein, 15 aa, Homo sapiens
QHGLPGCEEAGTARV

SEQ ID NO: 649 - protein, 15 aa, Homo sapiens
EAGTARVPSLHLHLH

SEQ ID NO: 650 - protein, 15 aa, Homo sapiens
SLHLHLHQAALGAGR

SEQ ID NO: 651 - protein, 15 aa, Homo sapiens
AALGAGRGRGWGEAC

SEQ ID NO: 652 - protein, 15 aa, Homo sapiens
RGWGEACAQVPPSRG

SEQ ID NO: 653 - protein, 15 aa, Homo sapiens
HYKLIQQPISLFSIT

SEQ ID NO: 654 - protein, 15 aa, Homo sapiens
ISLFSITDRLHKTFS

SEQ ID NO: 655 - protein, 15 aa, Homo sapiens
RLHKTFSQLPSVHLC

SEQ ID NO: 656 - protein, 15 aa, Homo sapiens
LPSVHLCSITFQWGH

SEQ ID NO: 657 - protein, 15 aa, Homo sapiens
ITFQWGHPPIFCSTN

SEQ ID NO: 658 - protein, 15 aa, Homo sapiens
PIFCSTNDICVTANF

SEQ ID NO: 659 - protein, 15 aa, Homo sapiens
ICVTANFCISVTFLK

SEQ ID NO: 660 - protein, 15 aa, Homo sapiens
ISVTFLKPCFLLHEA

SEQ ID NO: 661 - protein, 11 aa, Homo sapiens
CFLLHEASASQ

SEQ ID NO: 662 - protein, 15 aa, Homo sapiens
RTALTHNQDFSIYRL

SEQ ID NO: 663 - protein, 15 aa, Homo sapiens
DFSIYRLCCKRGSLC

SEQ ID NO: 664 - protein, 15 aa, Homo sapiens
CKRGSLCHASQARSP

SEQ ID NO: 665 - protein, 15 aa, Homo sapiens
ASQARSPAFPKPVRP

SEQ ID NO: 666 - protein, 15 aa, Homo sapiens
FPKPVRPLPAPITRI

SEQ ID NO: 667 - protein, 15 aa, Homo sapiens
PAPITRITPQLGGQS

SEQ ID NO: 668 - protein, 15 aa, Homo sapiens
PQLGGQSDSSQPLLT

SEQ ID NO: 669 - protein, 15 aa, Homo sapiens
SSQPLLTTGRPQGWQ

SEQ ID NO: 670 - protein, 15 aa, Homo sapiens
GRPQGWQDQALRHTQ

SEQ ID NO: 671 - protein, 15 aa, Homo sapiens
QALRHTQQASPASCA

SEQ ID NO: 672 - protein, 15 aa, Homo sapiens
ASPASCATITIPIHS

SEQ ID NO: 673 - protein, 15 aa, Homo sapiens
ITIPIHSAALGDHSG

SEQ ID NO: 674 - protein, 15 aa, Homo sapiens
ALGDHSGDPGPAWDT

SEQ ID NO: 675 - protein, 15 aa, Homo sapiens
PGPAWDTCPPLPLTT

SEQ ID NO: 676 - protein, 15 aa, Homo sapiens
PPLPLTTLIPRAPPP

SEQ ID NO: 677 - protein, 15 aa, Homo sapiens
IPRAPPPYGDSTARS

SEQ ID NO: 678 - protein, 16 aa, Homo sapiens
GDSTARSWPSRCGPLG

SEQ ID NO: 679 - protein, 15 aa, Homo sapiens
YAYKDFLWCFPFSLV

SEQ ID NO: 680 - protein, 15 aa, Homo sapiens
CFPFSLVFLQEIQIC

SEQ ID NO: 681 - protein, 15 aa, Homo sapiens
LQEIQICCHVSCLCC

SEQ ID NO: 682 - protein, 15 aa, Homo sapiens
HVSCLCCICCSTRIC

SEQ ID NO: 683 - protein, 15 aa, Homo sapiens
CCSTRICLGCLLELF

SEQ ID NO: 684 - protein, 15 aa, Homo sapiens
GCLLELFLSRALRAL

SEQ ID NO: 685 - protein, 15 aa, Homo sapiens
SRALRALHVLWNGFQ

SEQ ID NO: 686 - protein, 11 aa, Homo sapiens
VLWNGFQLHCQ

SEQ ID NO: 687 - protein, 15 aa, Homo sapiens
YEAGMTLGEKFRVGN

SEQ ID NO: 688 - protein, 16 aa, Homo sapiens
EKFRVGNCKHLKMTRP

SEQ ID NO: 689 - protein, 15 aa, Homo sapiens
WGMELAASRRFSWDH

SEQ ID NO: 690 - protein, 15 aa, Homo sapiens
RRFSWDHHSAGGPPR

SEQ ID NO: 691 - protein, 15 aa, Homo sapiens
SAGGPPRVPSVRSGA

SEQ ID NO: 692 - protein, 15 aa, Homo sapiens
PSVRSGAAQVQPKDP

SEQ ID NO: 693 - protein, 15 aa, Homo sapiens
QVQPKDPLPLRTLAG

SEQ ID NO: 694 - protein, 15 aa, Homo sapiens
PLRTLAGCLARTAHL

SEQ ID NO: 695 - protein, 15 aa, Homo sapiens
LARTAHLRPGAESLP

SEQ ID NO: 696 - protein, 14 aa, Homo sapiens
PGAESLPQPQLHCT

SEQ ID NO: 697 - protein, 15 aa, Homo sapiens
NSKMALNSLNSIDDA

SEQ ID NO: 698 - protein, 15 aa, Homo sapiens
LNSIDDAQLTRIAPP

SEQ ID NO: 699 - protein, 15 aa, Homo sapiens
LTRIAPPRSHCCFWE

SEQ ID NO: 700 - protein, 15 aa, Homo sapiens
APPRSHCCFWEVNAP

SEQ ID NO: 701 - protein, 15 aa, Homo sapiens
LWFQSSELSPTGAPW

SEQ ID NO: 702 - protein, 15 aa, Homo sapiens
SPTGAPWPSRRPTWR

SEQ ID NO: 703 - protein, 15 aa, Homo sapiens
SRRPTWRGTTVSPRT

SEQ ID NO: 704 - protein, 15 aa, Homo sapiens
TTVSPRTATSSARTC

SEQ ID NO: 705 - protein, 15 aa, Homo sapiens
TSSARTCCGTKWPSS

SEQ ID NO: 706 - protein, 15 aa, Homo sapiens
GTKWPSSQEAALGLG

SEQ ID NO: 707 - protein, 15 aa, Homo sapiens
EAALGLGSGLLRFSC

SEQ ID NO: 708 - protein, 13 aa, Homo sapiens
GLLRFSCGTAAIR

SEQ ID NO: 709 - protein, 15 aa, Homo sapiens
QNLQNGGGSRSSATL

SEQ ID NO: 710 - protein, 15 aa, Homo sapiens
QWQHYHRSGEAAGTP

SEQ ID NO: 711 - protein, 14 aa, Homo sapiens
GEAAGTPLWRPTRN

SEQ ID NO: 712 - protein, 16 aa, Homo sapiens
TEYNQKLQVNQFSESK

SEQ ID NO: 713 - DNA, 32767 bp, Artificial sequence
  recombinant GAd20
catcatcaataatataccttattttggattgaggccaatatgataatgaggtgggcggggcgaggcggggcgggtgacgtaggacgcgcgagtagggttgggaggtgtggcggaagtgtggcatttgcaagtgggaggagctgacatgcaatcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcgccgtcatctgacgggccgccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgagatcttccgtttatctaggtaccagatatcagaattcggatcccgcgacttcgccgccatgggccagaaagagcagatccacacactgcagaaaaacagcgagcggatgagcaagcagctgaccagatcttctcaggccgtgcagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgccttcaccaagagaagctttgccgtgaccgagcggatcatcgactattgggctcaaaaagagaagggcagcagcagcttcctgcggcctagctgtgattattgggcccagaaagaaaagatcagcatccccagaacacacctgtgcctggtgctgggagtgctgtctggacactctggcagcagactgtatgaggccggcatgacactcggcggcaagatcctgttcttcctgttcctgctgctccctctgagccccttcagcctgatcttcaccgagatcagctgctgcaccctgagcagcgaggaaaacgagtacctgcctagacctgagtggcagctgcaggtccccttcagagagctgaagaacgtttccgtgctggaaggcctgagacagggcagacttggcggcccttgtagctgtcactgccccagacctagtcaggccagactgacacctgtggatgtggccggacctttcctgtgtctgggagatcctggcctgtttccacctgtgaagtccagcatcacaggcggcaagtccacatgttctgcccctggacctcagagcctgcctagcacacccttcagcacataccctcagtgggtcatcctgatcaccgaactcggcatggaatgcaccctgggacaagtgggagccccatctcctagaagagaagaggatggctggcgcggaggccactctagattcaaagctgatgtgcccgctcctcagggcccttgttggggaggacaacctggatctgccccatcttctgccccacctgaacagtccctgctggattggaagttcgagatgagctacaccgtcggcggacctccacctcatgttcatgccagacctcggcactggaaaaccgacagagatggccacagctacaccagcaaagtgaactgcctcctgctgcaggatggcttccacggctgtgtgtctattactggcgccgctggcagacggaacctgagcatctttctgtttctgatgctgtgcaagctcgagttccacgcctgcaagatccagaacaagaactgccccgacttcaagaagttcgacggcccttgcggagaaagaggcggaggcagaacagctagagccctttgggctagaggcgacagcgttctgacaccagctctggaccctcagacacctgttagggcccctagcctgacaagagctgccgccgctgtgcactacaagctgatccagcagccaatcagcctgttcagcatcaccgaccggctgcacaagacattcagccagctgccaagcgtgcacctgtgctccatcaccttccagtggggacaccctcctatcttttgctccaccaacgacatctgcgtgaccgccaacttctgtatcagcgtgaccttcctgaagccttgctttctgctgcacgaggccagcgcctctcagtgccacttgtttctgcagccccaagtgggcacacctcctccacatacagcctctgctagagcacctagcggccctccacatcctcacgaatcttgtcctgccggaagaaggcctgccagagccgctcaaacatgtgccagacgacagcacggactgcctggatgtgaagaggctggaacagccagagtgcctagcctgcacctccatctgcatcaggctgctcttggagccggaagaggtagaggatggggcgaagcttgtgctcaggtgccaccttctagaggcgtgctgagattcctggacctgaaagtgcgctacctgcacagccagtggcagcactatcacagatctggcgaagccgccggaacacccctttggaggccaacaagaaacgtgcccttccgggaactgaagaaccagagaacagctcagggcgctcctggaatccaccatgctgcttctccagtggccgccaacctgtgtgatcctgccagacatgcccagcacaccaggattccttgtggcgctggacaagtgcgcgctggaagaggacctgaagcaggcggaggtgttctgcaacctcaaagacccgctcctgagaagcctggctgcccttgcagaagaggacagcctagactgcacaccgtgaaaatgtggcgagccgtggccatgatggtgcccgatagacaggtccactacgactttggactgggcgtgccaggcgatagcactcggagagccgtcagacggatgaacaccttttacgaagccgggatgaccctgggcgagaagttcagagtgggcaactgcaagcacctgaagatgacccggcctaacagcaagatggccctgaatagcgaggccctgtctgtggtgtctgaatgtggcgcctctgcctgtgacgtgtccctgatcgctatggactccgcctttgtgcagggcaaagactggggcgtgaagaagttcatccggcgggacttctacgcctacaaggacttcctgtggtgcttccccttctctctggtgttcctgcaagagatccagatctgctgtcatgtgtcctgcctgtgctgcatctgctgtagcaccagaatctgcctgggctgtctgctggaactgttcctgagcagagccctgagagcactgcacgtgctgtggaacggattccagctgcactgccagaccgagtacaaccagaaactgcaagtgaaccagttcagcgagagcaagagcctgtaccaccgggaaaagcagctcattgccatggacagcgccatctgcgaagagagaggcgccgcaggatctctgatctcctgcgaaacaatgcccgccatcctgaagctgcagaagaattgcctcctaagcctgcgaaccgctctgacacacaaccaggacttcagcatctacagactgtgttgcaagcggggctccctgtgccatgcaagccaagctagaagccccgcctttcctaaacctgtgcgacctctgccagctccaatcaccagaattacccctcagctcggcggccagagcgattcatctcaacctctgctgaccaccggcagacctcaaggctggcaagaccaagctctgagacacacccagcaggctagccctgcctcttgtgccaccatcacaatccccatccactctgccgctctgggcgatcattctggcgatcctggaccagcctgggacacatgtcctccactgccactcacaacactgatccctagggctcctccaccttacggcgattctaccgctagaagctggcccagcagatgtggaccactcggaggcaacacaaccctccagcaactgggagaagcctctcaggctcctagcggctctctgatccctctcagactgcctctcctgtgggaagttcggggccagcctgattcttttgccgcactgcacagctccctgaacgagctgggagagatcgctagagagctgcaccagttcgccttcgacctgctgatcaagagccacttcgtgcaaggcaaggattggggcctcaaaaagtttatccgcagagacttctggggcatggaactggccgccagcagaagattcagctgggatcatcatagcgcaggcggcccacctagagtgccatctgttagaagcggagctgcccaggtgcagcctaaagatcctctgccactgagaacactggccggctgccttgctagaacagcccatcttagacctggcgccgagtctctgcctcagccacaactgcactgtaccctgtggttccagtccagcgagctgtctcctactggtgccccttggccatctagacgccctacttggagaggcaccaccgtgtcaccaagaaccgccacaagcagcgccagaacctgttgtggcacaaagtggccctccagccaagaagccgctctcggacttggaagcggactgctgaggttctcttgtggaaccgccgccattcggaagatgcactttagcctgaaagaacaccctccaccaccttgtcctccagaggctttccaaagagctgctggcgaaggcggacctggtagaggtggtgctagaagaggtgctagggtgctgcagagcccattctgtagagcaggcgcaggcgaatggctgggccatcagagtctgagacatgtcgtcggctacggccacctggatacaagcggaagcagctctagctccagctggcctaactcaaaaatggctctgaacagcctgaactccatcgacgacgcccagctgacaagaatcgcccctcctagatctcactgctgcttttgggaagtgaacgccccaagccatcaccatcaccaccattagtaaaggcgcgcctagcggccgcgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgggccgcgatcgcgcttaggcctgaccatctggtgctggcctgcaccagggccgagtttgggtctagcgatgaggataccgattgaggtgggtaaggtgggcgtggctagcagggtgggcgtgtataaattgggggtctaaggggtctctctgtttgtcttgcaacagccgccgccatgagcgacaccggcaacagctttgatggaagcatctttagtccctatctgacagtgcgcatgcctcactgggccggagtgcgtcagaatgtgatgggttccaacgtggatggacgtcccgttctgccttcaaattcgtctactatggcctacgcgaccgtgggaggaactccgctggacgccgcgacctccgccgccgcctccgccgccgccgcgaccgcgcgcagcatggctacggacctttacagctctttggtggcgagcagcgcggcctctcgcgcgtctgctcgggatgagaaactgactgctctgctgcttaaactggaagacttgacccgggagctgggtcaactgacccagcaggtttccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattttttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgcttagctgggaggggtgcatccggggggatatgatgtgcatcttggactggatttttaggttggctatgttcccgcccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaacgtttctggcatcgctcacatcgtagttatgttccagggtgaggtcatcataggacatctttacgaatcgggggcgaagggtcccggactgggggatgatggtaccctcgggccccggggcgtagttcccctcacagatctgcatctcccaggctttcatttcagagggagggatcatatccacctgcggggcgatgaaaaagacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttgagcatatccctgacgtggatgttctccctgaccagttccgccagaaggcgctcgccgcccagcgaaagcagctcttgcaaggaagcaaaatttttcagcggtttcaggccatcggccgtgggcatgtttttcagcgtctgggtcagcagctccagcctgtcccagagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcactggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggccgagcagaccgtctcgcattccaccagccaagtgagttccgggcggtcagggtcaaaaaccaggttgcccccatgctttttgatgcgtttcttaccttggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccaacgggggggtataaaagggggtgggtgccctttcatcttcactctcttccgcatcgctgtctgcgagagccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttatcaagcttggtggcgaatgacccgtagagggcgttggagagcagcttggcgatggagcgcagggtctggtttttgtcgcggtcggctcgctccttggccgcgatgttgagttgcacgtactcgcgggccacgcacttccactcggggaacacggtggtgcgctcgtctgggatcaggcgcaccctccagccgcggttgtgcagggtgaccatgtcgacgctggtggcgacctcaccgcgcagacgctcgttggtccagcagaggcggccgcccttgcgcgagcagaaggggggtagggggtccagctggtcctcgtttggggggtccgcgtcgatggtaaagaccccggggagcaggcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggataccgaggtaggtggggtagcagcgccccccgcggatgctggcgcgcacgtagtcatagagctcgtgggagggggccagcatgttgggcccgaggttggtgcgctgggggcgctcggcgcggaagacgatctgcctgaagatggcgtgggagttggaggagatggtgggccgctggaagacgttgaagcttgcttcttgcaagcccacggagtccctgacgaaggaggcgtaggactcgcgcagcttgtgcaccagctcggcggtgacctggacgtcgagcgcacagtagtcgagggtctcgcggatgatgtcatacctatcctcccccttctttttccacagctcgcggttgaggacgaactcttcgcggtctttccagtactcttggaggggaaacccgtccgtgtccgaacggtaagagcctagcatgtagaactggttgacggcctggtaggggcagcagcccttctccacgggcagcgcgtaggcctgcgccgccttgcggagggaggtgtgggtgagggcgaaagtgtccctgaccatgactttgaggtattgatgtctgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtgtcccacgatgtagagctccaggaagcggggctggcccttgatggaggggagctttttaagttcctcgtaggtaagctcctcgggcgattccaggccgtgctcctccagggcccagtcttgcaagtgagggttggccgccaggaaggatcgccagaggtcgcgggccatgagggtctgcaggcggtcgcggaaggttctgaactgccgccccacggccattttttcgggggtgatgcagtagaaggtgagggggtctttctcccaggggtcccatctgagctctcgggcgaggtcgcgcgcggcagcgaccagagcctcgtcgccccccagtttcatgaccagcatgaagggcacgagttgcttgccaaaggctcccatccaagtgtaggtttctacatcgtaggtgacaaagaggcgctccgtgcgaggatgagagccgattgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttttactttggttgtctggccgccagcatctgtctcctggagggcgatggtggaacagaccaccacgccgcgagagccgcaggtccagatctcggcgctcggcgggcggagtttgatgacgacatcgcgcacattggagctgtccatggtctccagctcccgcggcggcaggtcagccgggagttccctggaggttcacctcgcagagacgggtcaaggcgcggacagtgttgagatggtatctgatttcaaggggcatgttggaggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgttcctgtatctggcgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatttctgccatgaactgctcgatctcttcctcctggagatctcctcgtccggcgcgctccacggtggccgccaggtcgttggagatgcgacccatgagctgcgagaaggcgttgagtccgccctcgttccagacccggctgtagaccacgcccccctcggcgtcgcgggcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagaacggatgagctcggcgacagtgtcgcgcacctcgcgctcgaaggccacggggggcgcttcttcctcttccacctcttcttccatgattgcttcttcttcttcctcagccgggacgggagggggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatttcgccgcggggcgggcggccgtgaggtagcgagacggcgctgactatgcatcttaacaattgctgtgtaggtacgccgccaagggacctgattgagtccagatccaccggatccgaaaacctttggaggaaagcgtctatccagtcgcagtcgcaaggtaggctgagcaccgtggcgggcgggggcgggtcgggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtaatcttgcatgagtctttccaccggcacttcttctccttcctcttcttcatctcgccggtggtttctcgcgccgcccatgcgcgtgaccccaaagcccctgagcggctgcagcagggccaggtcggcgaccacgcgctcggccaagatggcctgctgtacctgagtgagggtcctctcgaagtcatccatgtccacgaagcggtggtaggcacccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgcctgcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcagccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttggatcggccggaaccgcggctaacgtgggctgtggcagccccgtcctcaggaccccgccagccgacttctccagttacgggagcgagccccttttgtttttttattttttagatgcatcccgtgctgcggcagatgcgcccctcgccccggcccgatcagcagcagcaacagcaggcatgcagacccccctctcctctccccgccccggtcaccacggccgcggcggccgtgtccggtgcggggggcgcgctggagtcagatgagccaccgcggcggcgacctaggcagtatctggacttggaagagggcgagggactggcgcggctgggggcgagctctccagagcgccacccgcgggtgcagttgaaaagggacgcgcgtgaggcgtacctgccgcggcaaaacctgtttcgcgaccgcgggggcgaggagcccgaggagatgcgggactgcaggttccaagcggggcgcgagctgcgccgcggcttggacagacagcgcctgctgcgcgaggaggactttgagcccgacacgcagacgggcatcagccccgcgcgcgcgcacgtggccgcggccgacctggtgaccgcctacgagcagacggtgaaccaggagcgcaacttccaaaaaagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccattaactattctatgctgagcctgggcaagttctacgctcgcaagatctacaagaccccctacgtgcccatagacaaggaggtgaagatagacagcttctacatgcgcatggcgctgaaggtgctaaccctgagcgacgacctgggagtgtaccgcaacgagcgcatccacaaggccgtgagcgccagccggcggcgcgagctgagcgaccgcgaactgatgcacagtctgcagcgcgcgctgaccggcgcgggcgagggcgacagggaggtcgagtcctactttgacatgggggccgacctgcactggcagccgagccgccgcgccctggaagcggcgggggcgtacggcggccccctggcggccgatgacgaggaagaggaggactatgagctagaggagggcgagtacctggaggactgacctggctggtggtgttttggtatagatgcaagatccgaacgtggcggacccggcggtccgggcggcgctgcagagccagccgtccggcattaactcctctgacgactgggccgcggccatgggtcgcatcatggccctgaccgcgcgcaaccccgaggccttcaggcagcagcctcaggctaaccggctggcggccatcttggaagcggtagtgcccgcgcgctccaaccccacccacgagaaggtgctggccatagtcaacgcgctggcggagagcagggccatccgggcagacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggcaacctgggctcgctggtggcgctaaacgccttccttagcacccagccggccaacgtaccgcgggggcaggaggactacaccaacttcttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaggagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcttcccagcggccgttcatcaaccgcctgatggactacttgcatcgggcggcggccgtgaaccccgagtacttcaccaatgccattctgaatccccactggatgccccctccgggtttctacaacggggacttcgaggtgcctgaggtcaacgatgggttcctctgggatgacatggatgacagtgtgttctcccccaacccgctgcgcgccgcgtctctgcgattgaaggagggctctgacagggaaggaccaaggagtctggcctcctccctggctctgggggcggtgggcgccacgggcgcggcggcgcggggcagcagccccttccccagcctggcggactctctgaatagcgggcgggtgagcaggccccgcttgctaggcgaggaggagtatctgaacaactccctgctgcagcccgtgagggacaaaaacgctcagcggcagcagtttcccaacaatgggatagagagcctggtggacaagatgtccagatggaagacgtatgcgcaggagtacaaggagtgggaggaccgccagccgcggcccctgccgccccctagacagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagagaaaaataaaaactcaccaaggccatggcgacgagcgttggttttttgttcccttccttagtatgcggcgcgcggcgatgttcgaggaggggcctcccccctcttacgagagcgcgatgggaatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagtacccagaccataaacctggacaacaggtcgaactggggcggcgacctgaagactatcctgcacaccaatatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggagagcgatatcggggtcaagtttgacaccagaaacttccgtctgggctgggaccctgtgaccgggctggtcatgccgggggtctacaccaacgaggcctttcatcccgatatagtgctcctgcccggctgtggggtggacttcacccagagccggctgagcaacctgctgggcgttcgcaagcggcaacctttccaggagggtttcaagatcacctatgaggatctggaggggggcaacattcccgcgctccttgatctggacgcctacgaggagagcttgaaacccgaggagagcgctggcgacagcggcgagagtggcgaggagcaagccggcggcggcggcagcgcgtcggtagaaaacgaaagtactcccgcagtggcggcggacgctgcggaggtcgagccggaggccatgcagcaggacgcagaggagggcgcgcaggaggacatgaacaatggggagatcaggggcgacactttcgccacccggggcgaagaaaaagaggcagaggcggcggcggcgacggcggaagccgaaaccgaggcagaggcagagcccgagaccgaagttatggaagacatgaatgatggagaacgtaggggtgacacgtttgccacccggggcgaagagaaggcggcggaggcagaagccgcggctgaggaggcggctgcggctgcggccaaggctgaggctgcggctgaggctaaggtcgaagccgatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcattgagggcagcacctttacccaataccgcagctggtacctggcttacaactacggcgacccggtcaagggggtgcgctcgtggaccctgctctgcacgccggacgtcacctgcggctccgagcagatgtactggtcgctgccaaacatgatgcaagacccggtgaccttccgttccacgcggcaggttagcaactttccggtggtgggcgccgaactgctgccagtacactccaagagtttttacaacgagcaggccgtctactcccagctgatccgccaggccacctctctgacccacgtgttcaatcgctttcccgagaaccagattttggcgcgcccgccggcccccaccatcaccaccgtcagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatccacccacacgctccaaaatcatgtccgtactcatctcgcccagcaacaacaccggctgggggctgcgcgcacccagcaagatgtttggaggggcaaggaagcgctccgaccagcaccccgtgcgcgtgcgcggccactaccgcgcgccctggggtgcgcacaagcgcgggcgcacagggcgcaccactgtggatgatgtcattgactccgtagtggagcaggcgcgccactacacacccggcgcgccgaccgcctccgccgtgtccaccgtggaccaggcgatcgaaagcgtggtacagggggcgcggcactatgccaaccttaaaagtcgccgccgccgcgtggcgcgccgccatcgccggagaccccgggctactgccgccgcgcgccttaccaaggctctgctcaagcgcgccaggcgaactggccaccgggccgccatgagggccgcacggcgggctgccgctgccgcgagcgccgtggccccgcgggcacgaaggcgcgcggccgctgccgccgccgccgccatttccagcttggcctcgacgcggcgcggtaacatatactgggtgcgcgactcggtgagcggcacacgtgtgcccgtgcgctttcgccccccacggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggaggattacaagccccgcaagctaaagcgggtcaaaaagaaaaagaaagatgatgacgttgacgaggcggtggagtttgtccgccgcatggcgcccaggcgccctgtgcagtggaagggtcggcgcgtgcagcgagtcctgcgccccggcaccgcggtggtctttacgcccggcgagcgttccacgcgcactttcaagcgggtgtacgatgaggtgtacggcgacgaggatctgttggagcaggccaaccatcgatttggggagtttgcatatgggaaacggcctcgcgagagtctaaaagaggacctgctggcgctaccgctggacgagggcaatcccaccccgagtctgaagccggtgaccctgcaacaggtgctgcctttgagcgcgcccagcgagcagaagcgagggttaaagcgcgagggcggggacctggcacccaccgtgcagttgatggtgcccaagcggcagaagctggaggacgtgctggagaaaatgaaagtagagcccgggatccagcccgagatcaaggtccgccctatcaagcaggtggcgcccggcgtgggagtccagaccgtggacgttaggattcccacggaggagatggaaacccaaaccgccactccctcttcggcagcaagcgccaccaccggcgccgcttcggtagaggtgcagacggacccctggctacccgccgccactatcgccgtcgccgccgccccccgttcgcgcggacgcaagagaaattatccagcggccagcgcgcttatgccccagtatgcgctgcatccatccatcgcgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgccttcgcttcccggtgccgggataccgaggaagaactcaccgccgcaggggcatggcgggcagcggtctccgcggcggccgtcgccatcgccggcgcgcaaagagcaggcgcatgcgcggcggtgtgttgcccctgctggtcccgctactcgccgcggcgatcggcgccgtgcccgggatcgcctccgtggccctgcaggcgtcccagaaacattgactcttgcaaccttgcaagcttgcatttttggaggaaaaaataaaaaagtctagactctcacgctcgcttggtcctgtgactattttgtagaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggtcagatgctgagagacaagttgaaagagcagaacttccaggagaaggtggcgcagggcctggcctctggcatcagcggggtggtggacatagctaaccaggccgtgcagaaaaagataaacagtcatctggacccccgccctcaggtggaggaaacgcctccagccatggagacggtgtctcccgagggcaaaggcgaaaagcgcccgcggcccgacagggaagagaccctggtgtcacacaccgaggagccgccctcttacgaggaggcagtcaaggccggcctgcccaccactcgccccatagctcccatggccaccggtgtggtgggtcacaggcaacacacccccgcaacactagatctgcccccgccgtccgagccgactcgccagccaaaggcggtgacggtgtccgctccctccacttccgccgccaacagagtgcctctgcgccgcgctgcgagcggcccccgggcctcgcgagtcagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcttactcttacaaggcgcggttcacgctggccgtgggcgacaaccgcgtgctggacatggcctccacttactttgacatccggggggtgctggacaggggccccacttttaagccctactcgggcactgcctacaaccccctggcccccaagggcgcccccaattcttgtgagtgggaacaagaggaaaatcaggtggtcgctgcagatgatgaacttgaagatgaagaagcgcaagcacaagaggaagcccctgtgaaaaaaattcatgtatatgctcaggcgcctctttctggcgaaaagatttccaaggatggtatccaaataggtactgaagtcgtaggagatacatctaaggacacttttgcagataaaacattccaacccgaacctcagataggcgagtctcagtggaacgaggctgatgccacagcagcaggaggtagagttttgaaaaagactacccctatgagaccttgctatggatcctatgccaggcctaccaatgccaacgggggtcaaggaattatggttgccaatgaacaaggagtgttggagtctaaagtagaaatgcaatttttctctaacaccacaacccttaatgcgcgggatggaaccggcaatcccgaaccaaaggtggtgttgtacagcgaagatgtccacttggaatctcccgatactcatctgtcttacaagcccaaaaaggatgatgttaatgccaaaatcatgttgggtcagcaagccatgcccaacagacccaacctcattggatttagagataatttcattgggcttatgttttacaacagcaccggtaacatgggagtgctggcgggtcaggcctctcagttgaatgctgtggtggacttgcaggatagaaacacagaactgtcatatcagcttctgcttgattcaattggggatagaaccagatacttctccatgtggaaccaggcagtggatagctatgatccagatgtcagaattattgaaaaccatgggactgaggatgaactgcccaactactgcttccctttgggcggcataggagttactgatacttatcaagggataaaaaataccaatggcaatggtcagtggaccaaagatgatcagttcgcggaccgcaacgaaataggggtgggaaacaacttcgccatggagatcaacatccaggccaacctttggagaaacttcctctatgcaaacgtggggctctacctgccagacaagctcaagtacaaccccaccaacgtggacatctctgacaaccccaacacctatgactacatgaacaagcgggtggtggcccctggcctggtggactgctttgtcaatgtgggagccaggtggtccctggactacatggacaacgtcaaccccttcaaccaccaccgcaatgcgggtctgcgctaccgctccatgatcctgggcaacgggcgctatgtgccctttcacatccaggtaccccagaagttctttgccatcaagaacctcctgctcctgcccggctcctacacctacgagtggaacttcaggaaggatgtgaacatggtcctacagagctctctgggcaatgaccttagggtggatggggccagcatcaagtttgacagcatcaccctctatgctacatttttccccatggcccacaacaccgcctccacgcttgaggccatgctgagaaacgacaccaacgaccagtcctttaatgactacctctctggggccaacatgctctacccaatcccagccaaggccaccaacgtgcccatctccatcccctctcgcaactgggccgcctttagaggctgggcctttacccgccttaagaccaaggagaccccctccctgggctcgggttttgatccctactttgtttactcgggatccatcccctacctggatggcaccttctacctcaaccacactttcaagaagatatccatcatgtatgactcctccgtcagctggccgggcaacgaccgcttgctcacccccaatgagttcgaggtcaagcgcgccgtggacggcgagggctacaacgtggcccagtgcaacatgaccaaggactggttcctggtgcagatgctggccaactacaacataggctaccagggcttttacatcccagagagctacaaggacaggatgtactccttcttcagaaatttccaacccatgagccgacaggtggtggacgagaccaattacaaggactatcaagccattggcatcacccaccagcacaacaactcgggtttcgtgggctacctggcgcccaccatgcgcgagggtcaggcctaccccgccaacttcccctaccccttgataggcaagaccgcggtcgacagcgtcacccagaaaaagttcctctgcgaccgcaccctctggcgcatccccttctctagcaacttcatgtccatgggtgcgctcacggacctgggccaaaacctgctttatgccaactctgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgtacgcccttctcagccggcaacgccaccacctaaggagacagcgccgccgccgcctgcatgacgggttccaccgagcaagagctcagggccattgccagagacctgggatgcggaccctattttttgggcacctatgacaaacgcttcccgggctttatctcccgagacaagctcgcctgcgccattgtcaacacggccgcgcgcgagaccgggggcgtgcactggctggcctttggctgggacccgcgctccaaaacttgctacctctttgacccctttggcttctccgatcagcgcctcaggcagatttatgagtttgagtacgaggggctgctgcgccgcagcgcgctcgcctcctcgcccgaccgctgcatcacccttgagaagtccaccgaaaccgtgcaggggccccactcggccgcctgcggtctcttctgttgcatgtttttgcacgcctttgtgcactggcctcagagtcccatggattgcaaccccaccatgaacttgctaaagggagtgcccaacgccatgctccagagcccccaggtccagcccaccctgcgccgcaaccaggaacagctttaccgcttcctggagcgccactccccctacttccgcagccacagcgcgcgcatccggggggccacctctttttgccacttgcaagaaaacatgcaagacggaaaatgatgtacagcatgcttttaataaatgtaaagactgtgcactttaattatacacgggctctttctggttatttattcaacaccgccgtcgccatttagaaatcgaaagggttctgccgtgcgtcgccgtgcgccacgggcagagacacgttgcgatactggaagcggctcgcccacttgaactcgggcaccaccatgcggggcagtggttcctcggggaagttctcgctccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattattcacgctggccagcaggctctcgtcgctgatcatgtcgctgtccagatcctccgcgttgctcagggcgaatggggtcatcttgcagacctgcctgcccaggaaaggcgggagcccaggcttgccgttgcagtcgcagcgcaggggcattagcaggtgcccacggcccgactgcgcctgcgggtacaacgcgcgcatgaaggcttcgatctgcctaaaagccacctgggtcttggctccctccgaaaagaacatcccacaggacttgctggagaactggttcgcgggacagctggcatcgtgcaggcagcagcgcgcgtcagtgttggcaatctgcaccacgttgcgaccccaccggtttttcactatcttggccttggaagcctgctcctttagcgcgcgctggccgttctcgctggtcacatccatctctatcacctgttccttgttgatcatgtttgtcccgtgcagacactttaggtcgccctccgtctgggtgcagcggtgctcccacagcgcgcaaccggtgggctcccaattcttgtgggtcacccccgcgtaggcctgcaggtaggcctgcaggaagcgccccatcatggtcataaaggtcttctggctcgtaaaggtcagctgcaggccgcgatgctcttcgttcagccaggtcttgcagatggcggccagcgcctcggtctgctcgggcagcatcttaaaatttgtcttcaggtcgttatccacgtggtacttgtccatcatggcacgcgccgcctccatgcccttctcccaggcggacaccatgggcaggcttagggggtttatcacttccagcggcgaggacaccgtactttcgatttcttcttcctccccctcttcccggcgcgcgcccccgctgttgcgcgctcttaccgcctgcaccaaggggtcgtcttcaggcaagcgccgcaccgagcgcttgccgcccttgacctgcttgatcagtaccggcgggttgctgaagcccaccatggtcagcgccgcctgctcttcttcgtcttcgctgtctaccactatttctggggaggggcttctccgctctgcggcaaaggcggcggatcgcttcttttttttcttgggagccgccgcgatggagtccgccacggcgaccgaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgcgctggtcttggtcccgactggccattgtatcctcctcctcctaggcagagagacataaggagtctatcatgcaagtcgagaaggaggagagcttaaccaccccctcagagaccgccgatgcgcccgccgtcgccgtcgcccccgctaccgccgacgcgcccgccacaccgagcgacacccccacggacccccccgccgacgcacccctgttcgaggaagcggccgtggagcaggacccgggctttgtctcggcagaggaggatttgcaagaggaggagaataaggaggagaagccctcagtgccaaaagatcataaagagcaagacgagcacgacgcagacgcacaccagggtgaagtcgggcggggggacggagggcatggcggcgccgactacctagacgaaggaaacgacgtgctcttgaagcacctgcatcgtcagtgcgccatcgtctgcgacgctctgcaggagcgcagcgaggtgcccctcagcgtggcggaggtcagccgcgcctacgagctcagcctcttttccccccgggtgcccccccgccgccgcgaaaacggcacatgcgagcccaacccgcgcctcaacttctaccccgcctttgtggtgcccgaggtcctggccacctatcacatcttctttcaaaattgcaagatccccatctcgtgccgcgccaaccgtagccgcgccgataagatgctggccctgcgccagggcgaccacatacctgatatcgccgctttggaagatgtgccaaagatcttcgagggtctggggcgcaacgagaagcgggcagcaaactctctgcaacaggaaaacagcgaaaatgagagtcacactggagcgctggtggagctggagggcgacaacgcccgcctggcggtgctcaagcgcagcatcgaggtcacccactttgcctaccccgcgctcaacctgccccccaaagtcatgaacgcggtcatggacgggctgatcatgcgccgcggccggcccctcgctccagatgcaaacttgcatgaggagaccgaggacggtcagcccgtggtcagcgacgagcagctgacgcgctggctggagagcgcggaccccgccgaactggaggagcggcgcaagatgatgatggccgcggtgctggtcaccgtagagctggagtgtctgcagcgcttcttcggtgaccccgagatgcagagaaaggtcgaggagaccctacactacaccttccgccagggctacgtgcgccaggcttgcaagatctccaacgtggagctcagcaacctggtgtcctacctgggcatcttgcatgaaaaccgccttgggcagagcgtgctacactccaccctgcgcggggaggcgcgccgcgactacgtgcgcgactgcgtttacctcttcctctgctacacctggcagacggccatgggggtctggcagcagtgcctggaggagcgcaacctcaaggagctggagaagcttctgcagcgcgcgctcaaagacctctggacgggcttcaacgagcgctcggtggccgccgcgctagccgacctcatcttccccgagcgcctgctcaaaaccctccagcaggggctgcccgacttcaccagccaaagcatgttgcaaaattttaggaactttatcctggagcgttctggcatcctacccgccacctgctgcgccctgcccagcgactttgtccccctcgtgtaccgcgagtgccccccgccgctgtggggccactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgaaatcaggttttacgaggaccaatcccgcccgcccaaggcggagctgaccgcctgcgtcatcacccagggcgagatcctaggccaattgcaagccatccaaaaagcccgccaagagtttttgctgaagaggggtcggggggtgtatctggacccccagtcgggtgaggagctcaacccggttcccccgctgccaccgccgcgggaccttgcttcccaggataagcatcgccatggctcccagaaagaagcagcagcggccgccgctgccgccgccccacatgctggaggaagaggaggaatactgggacagtcaggcagaggaggtttcggacgaggaggagccggagacggagatggaagagtgggaggaggacagcttagacgaggaggcttccgaagccgaagaggcaggcgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagaggcggccagcggcggcggcgcccgtttcggtgcctaggaagacccagggcaagacttcagccaagaaactcgcggcgaccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgacatccgcacccatcgaaaccaaatattggtgaagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcagagctcgtcctcggcgccgcgctcgggcggcatcgggactctccagttcgtgcaggagtttgtgccctcggtctacttcaaccccttctcgggctctcccggtcgctacccggaccagtttatcccgaactttgacgccgcgagggactcggtggacggctacgactgaatgtcgggtggacccggtgcagagcaacttcgcctgaagcaccttgaccactgccgccgccctcagtgctttgcccgctgtcagaccggtgagttccagtacttttccctgcccgactcgcacccggacggcccggcgcacggggtgcgctttttcatcccgagtcaggtccgctctaccctaatcagggagttcaccgcccgtcccctactggcggagttggaaaaggggccttctatcctaaccattgcctgcatttgctctaaccctggattacaccaagatctttgctgtcatttgtgtgctgagtataataaaggctgagatcagaatctactcggaccttatccctttcaattgatcataactgtaatcaataaaaaatcacttacttgaaatctgatagcaagactctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttacagatgaaacgcgcgagatcgtctgacgagaccttcaaccccgtgtacccctacgataccgagatcgctccgacttctgtccctttccttacccctccctttgtatcatccgcaggaatgcaagaaaatccagctggggtgctgtccctgcacctgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaaccctttttgccactccccccctagcggtcagtggcgacaaccttactgtgcagtctcaggcccctcttactttggaagactcaaaactaactctggccaccaaaggacccctaactgtgtccgaaggcaaacttgtcctagaaacagagcctcccctgcatgcaagtgacagcagtagcctgggccttagcgtcacggccccacttagcattaacaatgacagcctaggactagacatgcaagcgcccatcagctctcgagatggaaaactggctctaacagtggcggcccccctaactgtggccgagggtatcaatgctttggcagtagccacaggtaatggtattggactaaatgaaaccaacacacacctgcaggcaaaactggtcgcgcccctaggctttgataccaacggcaacattaagctaagcgtcgcaggaggcatgaggctaaacaataacacactgatactagatgtaaactacccatttgaggctcaaggccaactgagcctaagagtgggctcgggcccactatatgtagattctagtagtcataacctaaccattagatgccttaggggattgtatgtaacatcttctaacaaccaaaacggtctagaggccaacattaaactaacaaaaggccttgtgtatgacggaaatgccatagcagttaatgttggcaaagggctggaatacagccctactggcacaacagaaaaacctatacagactaaaataggtctaggcatggagtatgacactgagggagccatgatgacaaaactaggctctggactaagctttgacaattcaggagccattgtggtgggaaacaaaaatgatgacaggcttactttgtggaccacaccggacccatcgcccaactgtcagatttactctgaaaaagatgctaaactaaccttggtactgactaaatgtggcagtcaggttg

SEQ ID NO: 714 - protein, 16 aa, Homo sapiens
GSTGCLTCRHHQTSHE

SEQ ID NO: 715 - protein, 15 aa, Homo sapiens
VVGRRHETAPQPLLV

SEQ ID NO: 716 - protein, 15 aa, Homo sapiens
APQPLLVPDRAGGEG

SEQ ID NO: 717 - protein, 9 aa, Homo sapiens
DRAGGEGGA

SEQ ID NO: 718 - protein, 15 aa, Homo sapiens
PVPTATPGVRSVTSP

SEQ ID NO: 719 - protein, 15 aa, Homo sapiens
VRSVTSPQGLGLFLK

SEQ ID NO: 720 - protein, 9 aa, Homo sapiens
GLGLFLKFI

SEQ ID NO: 721 - protein, 15 aa, Homo sapiens
KENDVREVCDVYLQM

SEQ ID NO: 722 - protein, 15 aa, Homo sapiens
CDVYLQMQIFFHFKF

SEQ ID NO: 723 - protein, 12 aa, Homo sapiens
IFFHFKFRSYFH

SEQ ID NO: 724 - protein, 15 aa, Homo sapiens
FARKMLEKVHRQHLQ

SEQ ID NO: 725 - protein, 14 aa, Homo sapiens
VHRQHLQLSHNSQE

SEQ ID NO: 726 - protein, 15 aa, Homo sapiens
MFLRKEQQVGPHSFS

SEQ ID NO: 727 - protein, 9 aa, Homo sapiens
VGPHSFSML

SEQ ID NO: 728 - protein, 15 aa, Homo sapiens
GLNLNTDRPGGYSYS

SEQ ID NO: 729 - protein, 15 aa, Homo sapiens
PGGYSYSIWWKNNAK

SEQ ID NO: 730 - protein, 9 aa, Homo sapiens
WWKNNAKNR

SEQ ID NO: 731 - protein, 15 aa, Homo sapiens
KVLNEIDAVVTVPPS

SEQ ID NO: 732 - protein, 15 aa, Homo sapiens
VVTVPPSLSTSQIPQ

SEQ ID NO: 733 - protein, 13 aa, Homo sapiens
STSQIPQGCCIIL

SEQ ID NO: 734 - protein, 15 aa, Homo sapiens
ANLKGTLQVRSGQAV

SEQ ID NO: 735 - protein, 10 aa, Homo sapiens
VRSGQAVSPR

SEQ ID NO: 736 - protein, 15 aa, Homo sapiens
LQAAASGQGKQGVPC

SEQ ID NO: 737 - protein, 15 aa, Homo sapiens
GKQGVPCPWGCCAYA

SEQ ID NO: 738 - protein, 15 aa, Homo sapiens
WGCCAYAESPRALIS

SEQ ID NO: 739 - protein, 15 aa, Homo sapiens
SPRALISGDAPSQVE

SEQ ID NO: 740 - protein, 15 aa, Homo sapiens
DAPSQVEREVPGPCL

SEQ ID NO: 741 - protein, 15 aa, Homo sapiens
EVPGPCLNTHSLSHR

SEQ ID NO: 742 - protein, 15 aa, Homo sapiens
THSLSHRSPQLPGLP

SEQ ID NO: 743 - protein, 14 aa, Homo sapiens
PQLPGLPHPKQPSV

SEQ ID NO: 744 - protein, 15 aa, Homo sapiens
GEVELSEGGEGQRHL

SEQ ID NO: 745 - protein, 15 aa, Homo sapiens
GEGQRHLAFPWACSG

SEQ ID NO: 746 - protein, 15 aa, Homo sapiens
FPWACSGPGWRGVCC

SEQ ID NO: 747 - protein, 15 aa, Homo sapiens
KMRAIQAEGGHGQAC

SEQ ID NO: 748 - protein, 15 aa, Homo sapiens
GGHGQACCGGAWGWA

SEQ ID NO: 749 - protein, 15 aa, Homo sapiens
GGAWGWAPGDGGPQG

SEQ ID NO: 750 - protein, 15 aa, Homo sapiens
GDGGPQGMLTHTLPT

SEQ ID NO: 751 - protein, 15 aa, Homo sapiens
LTHTLPTLGFQSAWT

SEQ ID NO: 752 - protein, 15 aa, Homo sapiens
GFQSAWTWRREDADR

SEQ ID NO: 753 - protein, 15 aa, Homo sapiens
RREDADRAWRTPKAC

SEQ ID NO: 754 - protein, 14 aa, Homo sapiens
WRTPKACASRRWSI

SEQ ID NO: 755 - protein, 15 aa, Homo sapiens
LLEPFRRGEPGPRGL

SEQ ID NO: 756 - protein, 15 aa, Homo sapiens
EPGPRGLLSGSSRGG

SEQ ID NO: 757 - protein, 15 aa, Homo sapiens
SGSSRGGEGPGRSIE

SEQ ID NO: 758 - protein, 15 aa, Homo sapiens
GPGRSIEAAPATPLP

SEQ ID NO: 759 - protein, 15 aa, Homo sapiens
APATPLPCCRKNPCR

SEQ ID NO: 760 - protein, 15 aa, Homo sapiens
CRKNPCRPQPSRFLP

SEQ ID NO: 761 - protein, 15 aa, Homo sapiens
QPSRFLPPRVLLVII

SEQ ID NO: 762 - protein, 15 aa, Homo sapiens
RVLLVIILPKLDCPK

SEQ ID NO: 763 - protein, 10 aa, Homo sapiens
PKLDCPKLGF

SEQ ID NO: 764 - protein, 15 aa, Homo sapiens
PSGRRTKRLVTLRSG

SEQ ID NO: 765 - protein, 15 aa, Homo sapiens
LVTLRSGCAIQCWHP

SEQ ID NO: 766 - protein, 15 aa, Homo sapiens
AIQCWHPRAGPVPSA

SEQ ID NO: 767 - protein, 15 aa, Homo sapiens
AGPVPSALPHTERPP

SEQ ID NO: 768 - protein, 15 aa, Homo sapiens
PHTERPPRLVRGAAD

SEQ ID NO: 769 - protein, 15 aa, Homo sapiens
LVRGAADPRTVTLGR

SEQ ID NO: 770 - protein, 15 aa, Homo sapiens
RTVTLGRSPAVMPRA

SEQ ID NO: 771 - protein, 9 aa, Homo sapiens
PAVMPRAPA

SEQ ID NO: 772 - protein, 15 aa, Homo sapiens
DCMLSEEGGQARRGG

SEQ ID NO: 773 - protein, 15 aa, Homo sapiens
GQARRGGSLCSLAAH

SEQ ID NO: 774 - protein, 15 aa, Homo sapiens
LCSLAAHTIASAARG

SEQ ID NO: 775 - protein, 15 aa, Homo sapiens
IASAARGRFLSRLSN

SEQ ID NO: 776 - protein, 15 aa, Homo sapiens
FLSRLSNFCAVVKAS

SEQ ID NO: 777 - protein, 16 aa, Homo sapiens
CAVVKASRGAPSCTWE

SEQ ID NO: 778 - protein, 15 aa, Homo sapiens
PEPRRLSPGEPRGRP

SEQ ID NO: 779 - protein, 15 aa, Homo sapiens
GEPRGRPRKGWGIWG

SEQ ID NO: 780 - protein, 15 aa, Homo sapiens
KGWGIWGLCGARVGP

SEQ ID NO: 781 - protein, 11 aa, Homo sapiens
CGARVGPKAWR

SEQ ID NO: 782 - protein, 15 aa, Homo sapiens
FVSLTAIQMASSATP

SEQ ID NO: 783 - protein, 15 aa, Homo sapiens
MASSATPWGRWPVAT

SEQ ID NO: 784 - protein, 15 aa, Homo sapiens
GRWPVATPTAACPRR

SEQ ID NO: 785 - protein, 15 aa, Homo sapiens
TAACPRRRPSSLPTG

SEQ ID NO: 786 - protein, 15 aa, Homo sapiens
PSSLPTGGDSASKKP

SEQ ID NO: 787 - protein, 15 aa, Homo sapiens
DSASKKPISRRAPWQ

SEQ ID NO: 788 - protein, 15 aa, Homo sapiens
SRRAPWQPWACPGRS

SEQ ID NO: 789 - protein, 15 aa, Homo sapiens
WACPGRSVNSAAPRA

SEQ ID NO: 790 - protein, 15 aa, Homo sapiens
NSAAPRAWCPPATTP

SEQ ID NO: 791 - protein, 15 aa, Homo sapiens
CPPATTPRTQSPSRD

SEQ ID NO: 792 - protein, 15 aa, Homo sapiens
TQSPSRDLRPRCLSS

SEQ ID NO: 793 - protein, 10 aa, Homo sapiens
RPRCLSSWSS

SEQ ID NO: 794 - protein, 15 aa, Homo sapiens
PVAIKPGTGPPNNSS

SEQ ID NO: 795 - protein, 15 aa, Homo sapiens
GPPNNSSIHGGSKRS

SEQ ID NO: 796 - protein, 15 aa, Homo sapiens
HGGSKRSENSYCRDL

SEQ ID NO: 797 - protein, 15 aa, Homo sapiens
NSYCRDLRGQLRAIC

SEQ ID NO: 798 - protein, 15 aa, Homo sapiens
GQLRAICCSSYSHDR

SEQ ID NO: 799 - protein, 15 aa, Homo sapiens
SSYSHDRHTTEERGS

SEQ ID NO: 800 - protein, 15 aa, Homo sapiens
TTEERGSRGRHVWRI

SEQ ID NO: 801 - protein, 15 aa, Homo sapiens
GRHVWRIRRLHTSGL

SEQ ID NO: 802 - protein, 15 aa, Homo sapiens
RLHTSGLPCCCHSGP

SEQ ID NO: 803 - protein, 15 aa, Homo sapiens
CCCHSGPHPRRLPDI

SEQ ID NO: 804 - protein, 15 aa, Homo sapiens
PRRLPDILRLVTSTK

SEQ ID NO: 805 - protein, 15 aa, Homo sapiens
RLVTSTKTDHTNTTE

SEQ ID NO: 806 - protein, 13 aa, Homo sapiens
DHTNTTEGTLDYL

SEQ ID NO: 807 - protein, 15 aa, Homo sapiens
KWNKNWTATLGALTI

SEQ ID NO: 808 - protein, 15 aa, Homo sapiens
TLGALTIRGHKLLCH

SEQ ID NO: 809 - protein, 15 aa, Homo sapiens
GHKLLCHLPHLLSSV

SEQ ID NO: 810 - protein, 16 aa, Homo sapiens
PHLLSSVQQTCRSSSR

SEQ ID NO: 811 - protein, 15 aa, Homo sapiens
ENASLVFTGSNSPIP

SEQ ID NO: 812 - protein, 15 aa, Homo sapiens
GSNSPIPACELSSHP

SEQ ID NO: 813 - protein, 15 aa, Homo sapiens
CELSSHPAHGISPWI

SEQ ID NO: 814 - protein, 15 aa, Homo sapiens
HGISPWIPSPGNEHF

SEQ ID NO: 815 - protein, 15 aa, Homo sapiens
SPGNEHFHGIKKQVK

SEQ ID NO: 816 - protein, 12 aa, Homo sapiens
GIKKQVKAIKVE

SEQ ID NO: 817 - protein, 15 aa, Homo sapiens
RLTQRLVQGWTPMEN

SEQ ID NO: 818 - protein, 15 aa, Homo sapiens
GWTPMENRWCGRRAG

SEQ ID NO: 819 - protein, 15 aa, Homo sapiens
WCGRRAGGQPASSST

SEQ ID NO: 820 - protein, 15 aa, Homo sapiens
QPASSSTRWTTCRAA

SEQ ID NO: 821 - protein, 15 aa, Homo sapiens
WTTCRAACLLTKWTA

SEQ ID NO: 822 - protein, 15 aa, Homo sapiens
LLTKWTAGRSQTSIG

SEQ ID NO: 823 - protein, 15 aa, Homo sapiens
ENSGNASRWLHVPSS

SEQ ID NO: 824 - protein, 15 aa, Homo sapiens
WLHVPSSSDDWLGWK

SEQ ID NO: 825 - protein, 16 aa, Homo sapiens
DDWLGWKKSSAITSNS

SEQ ID NO: 826 - protein, 15 aa, Homo sapiens
KGSVERRSVSLGHPA

SEQ ID NO: 827 - protein, 15 aa, Homo sapiens
VSLGHPAEGWAWAER

SEQ ID NO: 828 - protein, 15 aa, Homo sapiens
GWAWAERSLQPGMTT

SEQ ID NO: 829 - protein, 15 aa, Homo sapiens
LQPGMTTANTGCLSF

SEQ ID NO: 830 - protein, 15 aa, Homo sapiens
NTGCLSFHHRGCLLP

SEQ ID NO: 831 - protein, 15 aa, Homo sapiens
HRGCLLPVLPKLHCG

SEQ ID NO: 832 - protein, 15 aa, Homo sapiens
LPKLHCGLGGLPLVR

SEQ ID NO: 833 - protein, 15 aa, Homo sapiens
GGLPLVRAKEIKRVQ

SEQ ID NO: 834 - protein, 15 aa, Homo sapiens
KEIKRVQRAGESSLP

SEQ ID NO: 835 - protein, 15 aa, Homo sapiens
AGESSLPVKGLLTVA

SEQ ID NO: 836 - protein, 15 aa, Homo sapiens
KGLLTVASAVIAVLW

SEQ ID NO: 837 - protein, 15 aa, Homo sapiens
AVIAVLWGRPSEVTG

SEQ ID NO: 838 - protein, 14 aa, Homo sapiens
RPSEVTGENEAQHD

SEQ ID NO: 839 - protein, 15 aa, Homo sapiens
FGLTTLAGRSSHGTS

SEQ ID NO: 840 - protein, 15 aa, Homo sapiens
RSSHGTSGLRAATHT

SEQ ID NO: 841 - protein, 15 aa, Homo sapiens
LRAATHTKSGDGGQG

SEQ ID NO: 842 - protein, 15 aa, Homo sapiens
SGDGGQGAARQCEKL

SEQ ID NO: 843 - protein, 15 aa, Homo sapiens
ARQCEKLLELARATR

SEQ ID NO: 844 - protein, 15 aa, Homo sapiens
ELARATRPWGRSTSA

SEQ ID NO: 845 - protein, 15 aa, Homo sapiens
WGRSTSASSRWTHRG

SEQ ID NO: 846 - protein, 15 aa, Homo sapiens
SRWTHRGYMCPPRCA

SEQ ID NO: 847 - protein, 11 aa, Homo sapiens
MCPPRCAVACW

SEQ ID NO: 848 - protein, 15 aa, Homo sapiens
IIDSDKIMAVCMGCL

SEQ ID NO: 849 - protein, 15 aa, Homo sapiens
DKIMAVCMGCLLTRH

SEQ ID NO: 850 - protein, 15 aa, Homo sapiens
AVCMGCLLTRHVQCQ

SEQ ID NO: 851 - protein, 15 aa, Homo sapiens
GCLLTRHVQCQAMEM

SEQ ID NO: 852 - protein, 13 aa, Homo sapiens
TRHVQCQAMEMQQ

SEQ ID NO: 853 - protein, 16 aa, Homo sapiens
LSIFLFLMLCKLEFHA

SEQ ID NO: 854 - protein, 15 aa, Homo sapiens
LLNAEDYRCAIHSKE

SEQ ID NO: 855 - protein, 15 aa, Homo sapiens
CAIHSKEIYLLSPSP

SEQ ID NO: 856 - protein, 15 aa, Homo sapiens
YLLSPSPHQAMDKFS

SEQ ID NO: 857 - protein, 15 aa, Homo sapiens
QAMDKFSLCCINCNL

SEQ ID NO: 858 - protein, 15 aa, Homo sapiens
CCINCNLCLHVFLLL

SEQ ID NO: 859 - protein, 15 aa, Homo sapiens
LHVFLLLLFFQNKDV

SEQ ID NO: 860 - protein, 15 aa, Homo sapiens
FFQNKDVWLISNIIL

SEQ ID NO: 861 - protein, 14 aa, Homo sapiens
LISNIILLWIYGGI

SEQ ID NO: 862 - protein, 15 aa, Homo sapiens
VETLENANSFSSGIQ

SEQ ID NO: 863 - protein, 15 aa, Homo sapiens
SFSSGIQPLLCSLIG

SEQ ID NO: 864 - protein, 12 aa, Homo sapiens
LLCSLIGLENPT

SEQ ID NO: 865 - protein, 15 aa, Homo sapiens
AGAGTISEGSVLHGQ

SEQ ID NO: 866 - protein, 15 aa, Homo sapiens
GSVLHGQRLECDARR

SEQ ID NO: 867 - protein, 15 aa, Homo sapiens
LECDARRFFGCGTTI

SEQ ID NO: 868 - protein, 14 aa, Homo sapiens
FGCGTTILAEWEHH

SEQ ID NO: 869 - protein, 15 aa, Homo sapiens
KEQILAVASLVSSQS

SEQ ID NO: 870 - protein, 15 aa, Homo sapiens
SLVSSQSIHPSWGQS

SEQ ID NO: 871 - protein, 12 aa, Homo sapiens
HPSWGQSPLSRI

SEQ ID NO: 872 - protein, 15 aa, Homo sapiens
QQLRIFCAAMASNED

SEQ ID NO: 873 - protein, 9 aa, Homo sapiens
AMASNEDFS

SEQ ID NO: 874 - protein, 15 aa, Homo sapiens
DGFSGSLFAVVTRRC

SEQ ID NO: 875 - protein, 15 aa, Homo sapiens
AVVTRRCYFLKWRTI

SEQ ID NO: 876 - protein, 15 aa, Homo sapiens
FLKWRTIFPQSLMWL

SEQ ID NO: 877 - protein, 15 aa, Homo sapiens
DLRRVATYCAPLPSS

SEQ ID NO: 878 - protein, 15 aa, Homo sapiens
CAPLPSSWRPGTGTT

SEQ ID NO: 879 - protein, 15 aa, Homo sapiens
RPGTGTTIPPRMRSC

SEQ ID NO: 880 - protein, 15 aa, Homo sapiens
LQERMELLACGAERG

SEQ ID NO: 881 - protein, 15 aa, Homo sapiens
ACGAERGAGGWGGGG

SEQ ID NO: 882 - protein, 15 aa, Homo sapiens
GGWGGGGGGGGGDRR

SEQ ID NO: 883 - protein, 15 aa, Homo sapiens
GGGGDRRGGGGSAPA

SEQ ID NO: 884 - protein, 16 aa, Homo sapiens
GGGSAPALADFAGGRG

SEQ ID NO: 885 - protein, 15 aa, Homo sapiens
WTDIVKQSVSTNCIS

SEQ ID NO: 886 - protein, 15 aa, Homo sapiens
VSTNCISIKKGSYTK

SEQ ID NO: 887 - protein, 15 aa, Homo sapiens
KKGSYTKLFSLVFLI

SEQ ID NO: 888 - protein, 16 aa, Homo sapiens
FSLVFLIFCWPLIIQL

SEQ ID NO: 889 - protein, 15 aa, Homo sapiens
LRYGALCNVSRISYF

SEQ ID NO: 890 - protein, 15 aa, Homo sapiens
VSRISYFSLTNIFNF

SEQ ID NO: 891 - protein, 15 aa, Homo sapiens
LTNIFNFVIKSLTAI

SEQ ID NO: 892 - protein, 15 aa, Homo sapiens
RKERNIRKSESTLRL

SEQ ID NO: 893 - protein, 15 aa, Homo sapiens
SESTLRLSPFPTPAP

SEQ ID NO: 894 - protein, 15 aa, Homo sapiens
PFPTPAPSGAPAAAQ

SEQ ID NO: 895 - protein, 15 aa, Homo sapiens
GAPAAAQGKVVRVPG

SEQ ID NO: 896 - protein, 15 aa, Homo sapiens
KVVRVPGPAGGLVPR

SEQ ID NO: 897 - protein, 15 aa, Homo sapiens
AGGLVPRDAGARLLP

SEQ ID NO: 898 - protein, 15 aa, Homo sapiens
AGARLLPPAGGPGGG

SEQ ID NO: 899 - protein, 15 aa, Homo sapiens
AGGPGGGAAAGEGRA

SEQ ID NO: 900 - protein, 15 aa, Homo sapiens
AAGEGRAGRGRFPSI

SEQ ID NO: 901 - protein, 15 aa, Homo sapiens
RGRFPSITEPRPRDL

SEQ ID NO: 902 - protein, 15 aa, Homo sapiens
EPRPRDLPPRVATGR

SEQ ID NO: 903 - protein, 15 aa, Homo sapiens
PRVATGRRAGGRRKG

SEQ ID NO: 904 - protein, 15 aa, Homo sapiens
AGGRRKGAGQGVRTR

SEQ ID NO: 905 - protein, 15 aa, Homo sapiens
GQGVRTRPLPASWPG

SEQ ID NO: 906 - protein, 15 aa, Homo sapiens
LPASWPGGRGPFRKG

SEQ ID NO: 907 - protein, 15 aa, Homo sapiens
RGPFRKGPRRLPLGS

SEQ ID NO: 908 - protein, 15 aa, Homo sapiens
RRLPLGSGPPAAGVQ

SEQ ID NO: 909 - protein, 15 aa, Homo sapiens
PPAAGVQRLRCSHLS

SEQ ID NO: 910 - protein, 15 aa, Homo sapiens
LRCSHLSRGPRRRRG

SEQ ID NO: 911 - protein, 15 aa, Homo sapiens
GPRRRRGRVCGRACV

SEQ ID NO: 912 - protein, 15 aa, Homo sapiens
VCGRACVSPPLPPRP

SEQ ID NO: 913 - protein, 15 aa, Homo sapiens
PPLPPRPPPVGLSAE

SEQ ID NO: 914 - protein, 15 aa, Homo sapiens
PVGLSAENLSWLSSG

SEQ ID NO: 915 - protein, 15 aa, Homo sapiens
LSWLSSGLPRACSWR

SEQ ID NO: 916 - protein, 15 aa, Homo sapiens
PRACSWREFSPETCA

SEQ ID NO: 917 - protein, 15 aa, Homo sapiens
FSPETCAFRLSGLDS

SEQ ID NO: 918 - protein, 15 aa, Homo sapiens
RLSGLDSKLSARVER

SEQ ID NO: 919 - protein, 15 aa, Homo sapiens
LSARVERDLGALRAP

SEQ ID NO: 920 - protein, 15 aa, Homo sapiens
LGALRAPGSRAAQGG

SEQ ID NO: 921 - protein, 15 aa, Homo sapiens
SRAAQGGGRVRGSRS

SEQ ID NO: 922 - protein, 15 aa, Homo sapiens
RVRGSRSEWKTRPWR

SEQ ID NO: 923 - protein, 15 aa, Homo sapiens
WKTRPWRPPPAWPLT

SEQ ID NO: 924 - protein, 15 aa, Homo sapiens
PPAWPLTRAGGPLPK

SEQ ID NO: 925 - protein, 15 aa, Homo sapiens
AGGPLPKNPFLESCS

SEQ ID NO: 926 - protein, 15 aa, Homo sapiens
PFLESCSETAQRRRV

SEQ ID NO: 927 - protein, 15 aa, Homo sapiens
TAQRRRVFSFSTPLS

SEQ ID NO: 928 - protein, 15 aa, Homo sapiens
SINKATITGKKDLEL

SEQ ID NO: 929 - protein, 15 aa, Homo sapiens
GKKDLELILHVSRKK

SEQ ID NO: 930 - protein, 15 aa, Homo sapiens
LHVSRKKPFLPRVNI

SEQ ID NO: 931 - protein, 15 aa, Homo sapiens
FLPRVNITPTPISCC

SEQ ID NO: 932 - protein, 15 aa, Homo sapiens
PTPISCCNLKMLKKF

SEQ ID NO: 933 - protein, 15 aa, Homo sapiens
LKMLKKFFLLYIIIS

SEQ ID NO: 934 - protein, 15 aa, Homo sapiens
LLYIIISIIDLTNCL

SEQ ID NO: 935 - protein, 15 aa, Homo sapiens
IDLTNCLSCYLEHFY

SEQ ID NO: 936 - protein, 15 aa, Homo sapiens
CYLEHFYRFTFFTDV

SEQ ID NO: 937 - protein, 10 aa, Homo sapiens
FTFFTDVHYF

SEQ ID NO: 938 - protein, 15 aa, Homo sapiens
PYYSALSGNSWVPST

SEQ ID NO: 939 - protein, 15 aa, Homo sapiens
NSWVPSTLESDPFGY

SEQ ID NO: 940 - protein, 15 aa, Homo sapiens
ESDPFGYVFSPLATR

SEQ ID NO: 941 - protein, 15 aa, Homo sapiens
FSPLATRPALNDQES

SEQ ID NO: 942 - protein, 15 aa, Homo sapiens
ALNDQESILWPTLTS

SEQ ID NO: 943 - protein, 15 aa, Homo sapiens
LWPTLTSVVSCALSC

SEQ ID NO: 944 - protein, 15 aa, Homo sapiens
VSCALSCPSLNLPEN

SEQ ID NO: 945 - protein, 15 aa, Homo sapiens
SLNLPENWLTLITGG

SEQ ID NO: 946 - protein, 15 aa, Homo sapiens
LTLITGGMKGGKKMK

SEQ ID NO: 947 - protein, 12 aa, Homo sapiens
KGGKKMKFTFRH

SEQ ID NO: 948 - protein, 15 aa, Homo sapiens
GLRNLGNTVRAILLS

SEQ ID NO: 949 - protein, 15 aa, Homo sapiens
VRAILLSFLSKRNVK

SEQ ID NO: 950 - protein, 15 aa, Homo sapiens
LSKRNVKWCWGWGKP

SEQ ID NO: 951 - protein, 15 aa, Homo sapiens
CWGWGKPTSLGKACG

SEQ ID NO: 952 - protein, 14 aa, Homo sapiens
SLGKACGRRALKLF

SEQ ID NO: 953 - protein, 15 aa, Homo sapiens
MEAENAGSLHFHEVL

SEQ ID NO: 954 - protein, 14 aa, Homo sapiens
LHFHEVLKMGHVKF

SEQ ID NO: 955 - protein, 15 aa, Homo sapiens
WTPIPVLTRWPLPHP

SEQ ID NO: 956 - protein, 15 aa, Homo sapiens
RWPLPHPPPWRRATS

SEQ ID NO: 957 - protein, 15 aa, Homo sapiens
PWRRATSCRMARSSP

SEQ ID NO: 958 - protein, 15 aa, Homo sapiens
RMARSSPSATSGSSV

SEQ ID NO: 959 - protein, 15 aa, Homo sapiens
ATSGSSVRRRCSSLP

SEQ ID NO: 960 - protein, 15 aa, Homo sapiens
RRCSSLPSWVWNLAA

SEQ ID NO: 961 - protein, 16 aa, Homo sapiens
WVWNLAASTRPRSTPS

SEQ ID NO: 962 - protein, 15 aa, Homo sapiens
LHPQRETFTPRWSGA

SEQ ID NO: 963 - protein, 15 aa, Homo sapiens
TPRWSGANYWKLAFP

SEQ ID NO: 964 - protein, 15 aa, Homo sapiens
YWKLAFPVGAEGTFP

SEQ ID NO: 965 - protein, 15 aa, Homo sapiens
GAEGTFPAAATQRGV

SEQ ID NO: 966 - protein, 11 aa, Homo sapiens
AATQRGVVRPA

SEQ ID NO: 967 - protein, 15 aa, Homo sapiens
MAGGVLRRLLCREPD

SEQ ID NO: 968 - protein, 15 aa, Homo sapiens
LLCREPDRDGDKGAS

SEQ ID NO: 969 - protein, 15 aa, Homo sapiens
DGDKGASREETVVPL

SEQ ID NO: 970 - protein, 15 aa, Homo sapiens
EETVVPLHIGDPVVL

SEQ ID NO: 971 - protein, 15 aa, Homo sapiens
IGDPVVLPGIGQCYS

SEQ ID NO: 972 - protein, 10 aa, Homo sapiens
GIGQCYSALF

SEQ ID NO: 973 - protein, 15 aa, Homo sapiens
VFFKRAAEGFFRMNK

SEQ ID NO: 974 - protein, 15 aa, Homo sapiens
GFFRMNKLKESSDTN

SEQ ID NO: 975 - protein, 15 aa, Homo sapiens
KESSDTNPKPYCMAA

SEQ ID NO: 976 - protein, 15 aa, Homo sapiens
KPYCMAAPMGLTENN

SEQ ID NO: 977 - protein, 15 aa, Homo sapiens
MGLTENNRNRKKSYR

SEQ ID NO: 978 - protein, 15 aa, Homo sapiens
NRKKSYRETNLKAVS

SEQ ID NO: 979 - protein, 13 aa, Homo sapiens
TNLKAVSWPLNHT

SEQ ID NO: 980 - protein, 13 aa, Homo sapiens
GWRGVCCAAVEPA

(19) United States
(12) Patent Application Publication
(10) Pub. No.: US 2023/0035403 A1
(43) Pub. Date: Feb. 2, 2023

(54) Method For Determining Responsiveness To Prostate Cancer Treatment

(71) Applicant: Janssen Biotech, Inc., Horsham, PA (US)

(72) Inventors: Denis A. Smirnov, Media, PA (US); Yashoda Rani Rajpurohit, North Wales, PA (US); Vipul Bhargava, Warrington, PA (US); Patrick Wilkinson, Collegeville, PA (US); Kai Fu, Chalfont, PA (US); Manuel Alejandro Sepulveda, West Windsor, NJ (US)

(21) Appl. No.: 17/366,769

(22) Filed: Jul. 2, 2021

Related U.S. Application Data
(60) Provisional application No. 63/048,463, filed on Jul. 6, 2020.

(51) Int. Cl. G01N 33/50; G01N 33/574; C12Q 1/686; C12Q 1/6806; A61K 35/768; C12N 7/00

(52) U.S. Cl. CPC G01N 33/5011; G01N 33/57434; C12N 7/00; C12Q 1/6806; A61K 35/768; C12Q 1/686

(57) ABSTRACT

Disclosed herein are methods of diagnosing and treating a subject with prostate cancer, as well as methods of monitoring the responsiveness of a subject having prostate cancer to a therapeutic agent.

### CROSS REFERENCE TO RELATED APPLICATIONS

This application claims priority to U.S. Provisional Application No. 63/048,463, filed Jul. 6, 2020, the disclosure of which is hereby incorporated by reference in its entirety.

### SEQUENCE LISTING

This application contains a Sequence Listing submitted via EFS-Web, the entire content of which is incorporated herein by reference in its entirety. The ASCII text file, created on 30 Jun. 2020, is named 103693.002547_SL.txt and is 554 kilobytes in size.

### BACKGROUND

Prostate cancer is the most common non-cutaneous malignancy in men and the second leading cause of death in men from cancer in the western world. Prostate cancer results from the uncontrolled growth of abnormal cells in the prostate gland. Once a prostate cancer tumor develops, androgens such as testosterone promote prostate cancer growth. At its early stages, localized prostate cancer is often curable with local therapy including, for example, surgical removal of the prostate gland and radiotherapy. However, when local therapy fails to cure prostate cancer, as it does in up to a third of men, the disease progresses into incurable metastatic disease.

For many years, the established standard of care for men with malignant castration-resistant prostate cancer (mCRPC) was docetaxel chemotherapy. More recently, abiraterone acetate (ZYTIGA®) in combination with prednisone has been approved for treating metastatic castrate resistant prostate cancer. Androgen receptor (AR)-targeted agents, such as enzalutamide (XTANDI®) have also entered the market for treating metastatic castrate resistant prostate cancer. Platinum-based chemotherapy has been tested in a number of clinical studies in molecularly unselected prostate cancer patients with limited results and significant toxicities. However, there remains a subset of patients who either do not respond initially or become refractory (or resistant) to these treatments. No approved therapeutic options are available for such patients.

### BRIEF SUMMARY

Provided herein are methods of diagnosing a subject with prostate cancer, the methods comprising: evaluating the presence of one or more prostate cancer neoantigens in a sample from the subject, the one or more prostate cancer neoantigens comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof, wherein the presence of the one or more prostate cancer neoantigens is indicative of prostate cancer in the subject.

Also disclosed are methods of treating prostate cancer in a subject, the methods comprising:

a) evaluating the presence of one or more prostate cancer neoantigens in a sample from the subject, the one or more prostate cancer neoantigens comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof; and

b) administering a therapeutically effective amount of a prostate cancer vaccine to the subject to thereby treat the prostate cancer.

Also disclosed are methods of treating prostate cancer in a subject, the methods comprising:

a) evaluating the presence of one or more prostate cancer neoantigens in a sample from the subject, the one or more prostate cancer neoantigens comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof; and;

b) evaluating expression of one or more prostate cancer biomarkers in a sample from the subject, wherein the one or more prostate cancer biomarkers comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, ROR1, FGF8, NKX2-2, EDIL3, RELN, FGF9, AKR1C4, CLUL1, KISSIR, CYP3A5, CYP17A1, SFRP4, HNF1A, CALCR, SYP, MSLN, or any combination thereof; and

c) administering a therapeutically effective amount of a prostate cancer vaccine to the subject.

Methods for monitoring responsiveness of a subject having prostate cancer to a therapeutic agent are also provided. The methods comprise:

a) evaluating expression of one or more prostate cancer biomarkers, wherein the one or more prostate cancer biomarkers comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, ROR1, FGF8, NKX2-2, EDIL3, RELN, FGF9, AKR1C4, CLUL1, KISSIR, CYP3A5, CYP17A1, SFRP4, HNF1A, CALCR, SYP, MSLN, or combinations thereof;

b) administering a therapeutic agent to the subject; and

c) evaluating the expression of the one or more prostate cancer biomarkers evaluated in step a), wherein a decrease in the expression of the one or more prostate cancer biomarkers compared to the expression in step a) is indicative of responsiveness to the therapeutic agent.

Further provided are methods for preparing a cDNA from a subject with prostate cancer useful for analyzing an expression of prostate cancer neoantigens, the method comprising:

a) extracting RNA from a sample from the subject;

b) producing amplified cDNA from the RNA extracted in step a) by: (i) reverse transcribing the extracted RNA to produce the cDNA, and (ii) amplifying the cDNA; and

c) analyzing the amplified cDNA produced in step b) for one or more prostate cancer neoantigens, wherein the cDNA encodes an amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1 depicts an exemplary chimeric read-through fusion between Gene A and Gene B. Neoantigenic peptide sequences arise at the breakpoint junction.

FIG. 2 depicts an exemplary gene fusion resulting from chromosomal alteration, such as DNA translocations.

FIG. 3 depicts exemplary splice variants with alternative 5′ or 3′ splice sites, retained introns, excluded exons, or alternative terminations or insertions.

FIG. 4 depicts an exemplary approach of identifying splice variants.

FIG. 5A illustrates a flow cytometry dot plot depicting TNFα⁺IFNγCD8⁺ T cell frequencies in PBMC samples after no stimulation (DMSO)

FIG. 5B illustrates a flow cytometry dot plot depicting TNFα⁺IFNγCD8⁺ T cell frequencies in PBMC samples after stimulating with CEF peptide.

FIG. 5C illustrates a flow cytometry dot plot depicting TNFα⁺IFNγCD8⁺ T cell frequencies in PBMC samples after stimulation with P16.

FIG. 5D illustrates a flow cytometry dot plot depicting TNFα⁺IFNγCD8⁺ T cell frequencies in PBMC samples after stimulation with P98.

FIG. 5E illustrates a flow cytometry dot plot depicting TNFα⁺IFNγCD8⁺ T cell frequencies in PBMC samples after stimulation with P3 self-antigen.

FIG. 6 illustrates the number of prostate cancer patients whose PBMC samples demonstrated a positive immune response to the specified neoantigens. P3, P6, P7, P9 and P92 represent self-antigens.

FIG. 7 illustrates the number of prostate cancer patients whose PBMC samples demonstrated a positive CD8⁺ immune response to the specified neoantigens.

FIG. 8 illustrates the number of prostate cancer patients whose PBMC samples demonstrated a positive CD4⁺ immune response to the specified neoantigens.

FIG. 9 illustrates an exemplary embodiment of the disclosed methods for monitoring responsiveness of a subject having prostate cancer to a therapeutic agent.

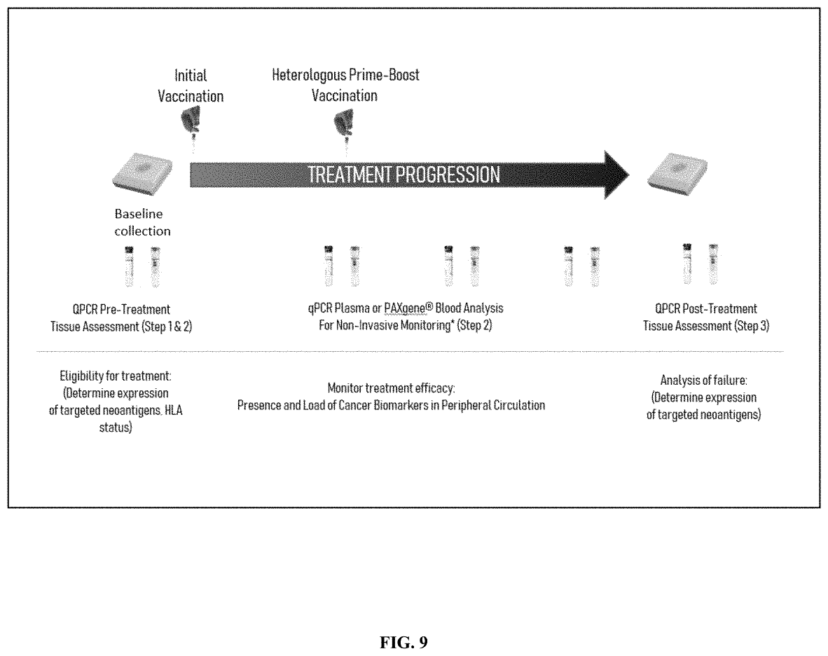

FIG. 10 illustrates the genes from exosome samples with AUC values larger than 0.55.

FIG. 11 illustrates the mean and standard deviation (error bar) of the accuracy, sensitivity, and specificity for the exosome samples.

FIG. 12 illustrates the genes from PAXgene samples with AUC values larger than 0.55.

FIG. 13 illustrates the mean and standard deviation (error bar) of the accuracy, sensitivity, and specificity for the PAXgene samples.

FIG. 14 illustrates the MC38 tumor volume (mm³) in mice immunized with GAd20-PCaNeoAg compared to mice that did not receive GAd20-PCaNeoAg immunization and mice implanted with the parental MC38 cell line that did not express the 10 prostate neoantigens.

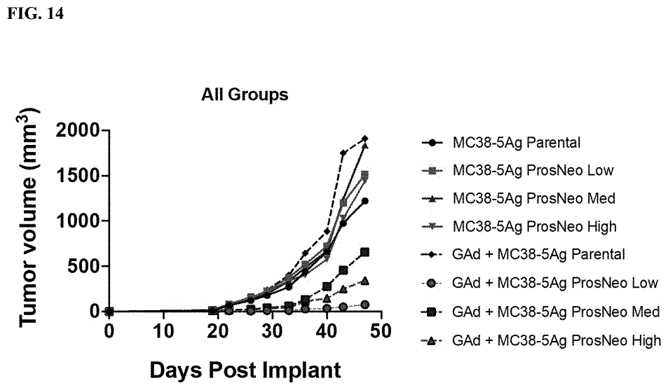

### DETAILED DESCRIPTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

All publications, including but not limited to patents and patent applications, cited in this specification are herein incorporated by reference as though fully set forth.

Although any methods and materials similar or equivalent to those described herein may be used in the practice for testing of the present disclosure, exemplary materials and methods are described herein. In describing and claiming the present disclosure, the following terminology will be used.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Throughout this text, the descriptions refer to methods of diagnosis and methods of treatment. Where the disclosure describes or claims a feature or embodiment associated with a method of diagnosis, such a feature or embodiment is equally applicable to the methods of treatment. Likewise, where the disclosure describes or claims a feature or embodiment associated with a method of treatment, such a feature or embodiment is equally applicable to the method of diagnosis.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include plural referents unless the content clearly dictates otherwise. Thus, for example, reference to “a cell” includes a combination of two or more cells, and the like.

The term “comprising” is intended to include examples encompassed by the terms “consisting essentially of” and “consisting of”; similarly, the term “consisting essentially of” is intended to include examples encompassed by the term “consisting of.”

As used herein, the phrase “and fragments thereof” when appended to a list includes fragments of one or more members of the associated list. The list may comprise a Markush group so that, as an example, the phrase “the group consisting of peptides A, B, and C, and fragments thereof” specifies or recites a Markush group including A, B, C, fragments of A, fragments of B, and/or fragments of C.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Immunogenic fragment” refers to a polypeptide that is recognized by cytotoxic T lymphocytes, helper T lymphocytes, or B cells when the fragment is in complex with MHC class I or MHC class II molecules.

“In-frame” refers to the reading frame of codons in a first polynucleotide being the same as the reading frame of codons in a second polynucleotide which are joined together to form a polynucleotide. In-frame polynucleotide encodes a polypeptide encoded by both the first polynucleotide and the second polynucleotide.

“Immunogenic” refers to a polypeptide that comprises one or more immunogenic fragments.

“Heterologous” refers to two or more polynucleotides or two or more polypeptides that are not found in the same relationship to each other in nature.

“Heterologous polynucleotide” refers to a non-naturally occurring polynucleotide that encodes two or more neoantigens as described herein.

“Heterologous polypeptide” refers to a non-naturally occurring polypeptide comprising two or more neoantigen polypeptides as described herein.

“Non-naturally occurring” refers to a molecule that does not exist in nature.

“Neoantigen” refers to a polypeptide that is present in prostate tumor tissue that has at least one alteration that makes it distinct from the corresponding wild-type polypeptide present in non-malignant tissue, e.g., via mutation in a tumor cell or post-translational modification specific to a tumor cell. A mutation can include a frameshift or nonframeshift insertion or deletion, missense or nonsense substitution, splice site alteration, aberrant splice variants, genomic rearrangement or gene fusion, or any genomic or expression alteration giving rise to the neoantigen.

“Recombinant” refers to polynucleotides, polypeptides, vectors, viruses and other macromolecules that are prepared, expressed, created or isolated by recombinant means.

“Vaccine” refers to a composition that comprises one or more immunogenic polypeptides, immunogenic polynucleotides or fragments, or any combination thereof intentionally administered to induce acquired immunity in the recipient (e.g. subject).

“Treat,” “treating,” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Prevent,” “preventing,” “prevention,” or “prophylaxis” of a disease or disorder means preventing that a disorder occurs in subject.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“In combination with” means that two or more therapeutic agents are administered to a subject together in a mixture, concurrently as single agents or sequentially as single agents in any order.

“Enhance” or “induce” when in reference to an immune response refers to increasing the scale and/or efficiency of an immune response or extending the duration of the immune response. The terms are used interchangeably with “augment.”

“Immune response” refers to any response to an immunogenic polypeptide or polynucleotide or fragment by the immune system of a vertebrate subject. Exemplary immune responses include local and systemic cellular as well as humoral immunity, such as cytotoxic T lymphocyte (CTL) responses, including antigen-specific induction of CD8⁺ CTLs, helper T-cell responses including T-cell proliferative responses and cytokine release, and B-cell responses including antibody response.

“Variant,” “mutant,” or “altered” refers to a polypeptide or a polynucleotide that differs from a reference polypeptide or a reference polynucleotide by one or more modifications, for example one or more substitutions, insertions, or deletions.

“About” means within an acceptable error range for the particular value as determined by one of skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Prime-boost” or “prime-boost regimen” refers to a method of treating a subject involving priming a T-cell response with a first vaccine followed by boosting the immune response with a second vaccine. The first vaccine and the second vaccine are typically distinct. These prime-boost immunizations elicit immune responses of greater height and breadth than can be achieved by priming and boosting with the same vaccine. The priming step initiates memory cells and the boost step expands the memory response. Boosting can occur once or multiple times.

Cancer cells produce neoantigens that result from genomic alterations and aberrant transcriptional programs. Neoantigen burden in patients has been associated with response to immunotherapy (Snyder et al., N Engl J Med. 2014 Dec. 4; 371(23):2189-2199. doi: 10.1056/NEJMoa1406498. Epub 2014 Nov. 19; Le et al., N Engl J Med. 2015 Jun. 25; 372(26):2509-20. doi: 10.1056/NEJMoa1500596. Epub 2015 May 30; Rizvi et al., Science. 2015 Apr. 3; 348(6230):124-8. doi: 10.1126/science.aaa1348. Epub 2015 Mar. 12; Van Allen et al., Science. 2015 Oct. 9; 350(6257):207-211. doi: 10.1126/science.aad0095. Epub 2015 Sep. 10). The disclosure is based, at least in part, on the identification of prostate neoantigens that are common in prostate cancer patients and hence can be utilized in diagnosing a subject with prostate cancer, treating prostate cancer, and monitoring responsiveness of a subject having prostate cancer to a therapeutic agent. One or more neoantigens or polynucleotides encoding the neoantigens of the disclosure may also be used for diagnostic or prognostic purposes.

### Methods of Diagnosis

Disclosed herein are methods of diagnosing a subject with prostate cancer. The methods comprise evaluating the presence of one or more prostate cancer neoantigens in a sample from the subject, the one or more prostate cancer neoantigens comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof, wherein the presence of the one or more prostate cancer neoantigens is indicative of prostate cancer in the subject.

The methods can comprise evaluating the presence of one or more prostate cancer neoantigens comprising the amino acid sequence of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, fragments of the preceding sequences, or any combination thereof.

In some embodiments, the methods comprise evaluating the presence of one or more prostate cancer neoantigens comprising the amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, 223, fragments of the preceding sequences, or any combination thereof.

In some embodiments, the methods comprise evaluating the presence of each of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, and 223.

The presence of the one or more prostate cancer neoantigens can be evaluated by, for example, PCR, quantitative PCR (qPCR), various forms of nucleic acid sequencing (including but not limited to Illumina, Ion Torrent, Pacific Bioscience, Oxford Nanopore platforms), and various hybridization-based approaches (including not limited to Affymetrix Gene Chip or Nanostring platforms).

In some embodiments, the presence of the one or more prostate cancer neoantigens is evaluated by qPCR. In some aspects, the methods further comprise, prior to performing the qPCR, extracting RNA from the sample from the subject and synthesizing cDNA from the extracted RNA. The RNA extracted from the subject can correspond to a polynucleotide sequence comprising SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 380, 382, 384, 386, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 503, 504, 505, 506, 507, 508, 509, 510, 511, 512, 513, 514, 515, 516, 517, 519, 520, 521, 522, 523, 524, 525, 528, 529, 530, 531, 532, 533, 534, 535, 536, 537, 538, 539, 540, fragments of the preceding sequences, or any combination thereof.

As used herein, “RNA . . . can correspond to a polynucleotide sequence comprising” refers to an RNA transcript generated from the DNA encoding the RNA or the RNA complement of a cDNA, wherein the DNA or cDNA comprise the listed sequence (i.e. SEQ ID NO).

The methods can comprise evaluating the presence of the prostate cancer neoantigen comprising the amino acid sequence of SEQ ID NO: 1 or a fragment thereof. In some embodiments, the polypeptide of SEQ ID NO: 1, or a fragment thereof, is encoded by the polynucleotide of SEQ ID NO: 2 or a fragment thereof.

The methods can comprise evaluating the presence of the prostate cancer neoantigen comprising the amino acid sequence of SEQ ID NO: 3 or a fragment thereof. In some embodiments, the polypeptide of SEQ ID NO: 3, or a fragment thereof, is encoded by the polynucleotide of SEQ ID NO: 4 or a fragment thereof.

The methods can comprise evaluating the presence of the prostate cancer neoantigen comprising the amino acid sequence of SEQ ID NO: 5 or a fragment thereof. In some embodiments, the polypeptide of SEQ ID NO: 5, or a fragment thereof, is encoded by the polynucleotide of SEQ ID NO: 6 or a fragment thereof.

The methods can comprise evaluating the presence of the prostate cancer neoantigen comprising the amino acid sequence of SEQ ID NO: 7 or a fragment thereof. In some embodiments, the polypeptide of SEQ ID NO: 7, or a fragment thereof, is encoded by the polynucleotide of SEQ ID NO: 8 or a fragment thereof.

The methods can comprise evaluating the presence of the prostate cancer neoantigen comprising the amino acid sequence of SEQ ID NO: 9 or a fragment thereof. In some embodiments, the polypeptide of SEQ ID NO: 9, or a fragment thereof, is encoded by the polynucleotide of SEQ ID NO: 10 or a fragment thereof.

The methods can comprise evaluating the presence of the prostate cancer neoantigen comprising the amino acid sequence of SEQ ID NO: 11 or a fragment thereof. In some embodiments, the polypeptide of SEQ ID NO: 11, or a fragment thereof, is encoded by the polynucleotide of SEQ ID NO: 12 or a fragment thereof.

The methods can comprise evaluating the presence of the prostate cancer neoantigen comprising the amino acid sequence of SEQ ID NO: 13 or a fragment thereof. In some embodiments, the polypeptide of SEQ ID NO: 13, or a fragment thereof, is encoded by the polynucleotide of SEQ ID NO: 14 or a fragment thereof.

The methods can comprise evaluating the presence of the prostate cancer neoantigen comprising the amino acid sequence of SEQ ID NO: 15 or a fragment thereof. In some embodiments, the polypeptide of SEQ ID NO: 15, or a fragment thereof, is encoded by the polynucleotide of SEQ ID NO: 16 or a fragment thereof.

The methods can comprise evaluating the presence of the prostate cancer neoantigen comprising the amino acid sequence of SEQ ID NO: 17 or a fragment thereof. In some embodiments, the polypeptide of SEQ ID NO: 17, or a fragment thereof, is encoded by the polynucleotide of SEQ ID NO: 18 or a fragment thereof.

The methods can comprise evaluating the presence of the prostate cancer neoantigen comprising the amino acid sequence of SEQ ID NO: 19 or a fragment thereof. In some embodiments, the polypeptide of SEQ ID NO: 19, or a fragment thereof, is encoded by the polynucleotide of SEQ ID NO: 20 or a fragment thereof. In some embodiments, the polypeptide of SEQ ID NO: 19, or a fragment thereof, is encoded by the polynucleotide of SEQ ID NO: 497 or a fragment thereof. In some embodiments, the polypeptide of SEQ ID NO: 19, or a fragment thereof, is encoded by the polynucleotide of SEQ ID NO: 538 or a fragment thereof.

The methods can comprise evaluating the presence of the prostate cancer neoantigen comprising the amino acid sequence of SEQ ID NO: 21 or a fragment thereof. In some embodiments, the polypeptide of SEQ ID NO: 21, or a fragment thereof, is encoded by the polynucleotide of SEQ ID NO: 22 or a fragment thereof.

The methods can comprise evaluating the presence of the prostate cancer neoantigen comprising the amino acid sequence of SEQ ID NO: 23 or a fragment thereof. In some embodiments, the polypeptide of SEQ ID NO: 23, or a fragment thereof, is encoded by the polynucleotide of SEQ ID NO: 24 or a fragment thereof. In some embodiments, the polypeptide of SEQ ID NO: 23, or a fragment thereof, is encoded by the polynucleotide of SEQ ID NO: 498 or a fragment thereof. In some embodiments, the polypeptide of SEQ ID NO: 23, or a fragment thereof, is encoded by the polynucleotide of SEQ ID NO: 539 or a fragment thereof.

The methods can comprise evaluating the presence of the prostate cancer neoantigen comprising the amino acid sequence of SEQ ID NO: 25 or a fragment thereof. In some embodiments, the polypeptide of SEQ ID NO: 25, or a fragment thereof, is encoded by the polynucleotide of SEQ ID NO: 26 or a fragment thereof.

The methods can comprise evaluating the presence of the prostate cancer neoantigen comprising the amino acid sequence of SEQ ID NO: 27 or a fragment thereof. In some embodiments, the polypeptide of SEQ ID NO: 27, or a fragment thereof, is encoded by the polynucleotide of SEQ ID NO: 28 or a fragment thereof.

The methods can comprise evaluating the presence of the prostate cancer neoantigen comprising the amino acid sequence of SEQ ID NO: 29 or a fragment thereof. In some embodiments, the polypeptide of SEQ ID NO: 29, or a fragment thereof, is encoded by the polynucleotide of SEQ ID NO: 30 or a fragment thereof.

The methods can comprise evaluating the presence of the prostate cancer neoantigen comprising the amino acid sequence of SEQ ID NO: 31 or a fragment thereof. In some embodiments, the polypeptide of SEQ ID NO: 31, or a fragment thereof, is encoded by the polynucleotide of SEQ ID NO: 32 or a fragment thereof.

The methods can comprise evaluating the presence of the prostate cancer neoantigen comprising the amino acid sequence of SEQ ID NO: 33 or a fragment thereof. In some embodiments, the polypeptide of SEQ ID NO: 33, or a fragment thereof, is encoded by the polynucleotide of SEQ ID NO: 34 or a fragment thereof.

The methods can comprise evaluating the presence of the prostate cancer neoantigen comprising the amino acid sequence of SEQ ID NO: 35 or a fragment thereof. In some embodiments, the polypeptide of SEQ ID NO: 35, or a fragment thereof, is encoded by the polynucleotide of SEQ ID NO: 36 or a fragment thereof.

The methods can comprise evaluating the presence of the prostate cancer neoantigen comprising the amino acid sequence of SEQ ID NO: 37 or a fragment thereof. In some embodiments, the polypeptide of SEQ ID NO: 37, or a fragment thereof, is encoded by the polynucleotide of SEQ ID NO: 38 or a fragment thereof.

The methods can comprise evaluating the presence of the prostate cancer neoantigen comprising the amino acid sequence of SEQ ID NO: 39 or a fragment thereof. In some embodiments, the polypeptide of SEQ ID NO: 39, or a fragment thereof, is encoded by the polynucleotide of SEQ ID NO: 40 or a fragment thereof.

The methods can comprise evaluating the presence of the prostate cancer neoantigen comprising the amino acid sequence of SEQ ID NO: 41 or a fragment thereof. In some embodiments, the polypeptide of SEQ ID NO: 41, or a fragment thereof, is encoded by the polynucleotide of SEQ ID NO: 42 or a fragment thereof.

The methods can comprise evaluating the presence of the prostate cancer neoantigen comprising the amino acid sequence of SEQ ID NO: 43 or a fragment thereof. In some embodiments, the polypeptide of SEQ ID NO: 43, or a fragment thereof, is encoded by the polynucleotide of SEQ ID NO: 44 or a fragment thereof.

The methods can comprise evaluating the presence of the prostate cancer neoantigen comprising the amino acid sequence of SEQ ID NO: 45 or a fragment thereof. In some embodiments, the polypeptide of SEQ ID NO: 45, or a fragment thereof, is encoded by the polynucleotide of SEQ ID NO: 46 or a fragment thereof.

The methods can comprise evaluating the presence of the prostate cancer neoantigen comprising the amino acid sequence of SEQ ID NO: 47 or a fragment thereof. In some embodiments, the polypeptide of SEQ ID NO: 47, or a fragment thereof, is encoded by the polynucleotide of SEQ ID NO: 48 or a fragment thereof.

The methods can comprise evaluating the presence of the prostate cancer neoantigen comprising the amino acid sequence of SEQ ID NO: 49 or a fragment thereof. In some embodiments, the polypeptide of SEQ ID NO: 49, or a fragment thereof, is encoded by the polynucleotide of SEQ ID NO: 50 or a fragment thereof.

The methods can comprise evaluating the presence of the prostate cancer neoantigen comprising the amino acid sequence of SEQ ID NO: 51 or a fragment thereof. In some embodiments, the polypeptide of SEQ ID NO: 51, or a fragment thereof, is encoded by the polynucleotide of SEQ ID NO: 52 or a fragment thereof.

The methods can comprise evaluating the presence of the prostate cancer neoantigen comprising the amino acid sequence of SEQ ID NO: 53 or a fragment thereof. In some embodiments, the polypeptide of SEQ ID NO: 53, or a fragment thereof, is encoded by the polynucleotide of SEQ ID NO: 54 or a fragment thereof.

The methods can comprise evaluating the presence of the prostate cancer neoantigen comprising the amino acid sequence of SEQ ID NO: 55 or a fragment thereof. In some embodiments, the polypeptide of SEQ ID NO: 55, or a fragment thereof, is encoded by the polynucleotide of SEQ ID NO: 56 or a fragment thereof.

The methods can comprise evaluating the presence of the prostate cancer neoantigen comprising the amino acid sequence of SEQ ID NO: 57 or a fragment thereof. In some embodiments, the polypeptide of SEQ ID NO: 57, or a fragment thereof, is encoded by the polynucleotide of SEQ ID NO: 58 or a fragment thereof.

The methods can comprise evaluating the presence of the prostate cancer neoantigen comprising the amino acid sequence of SEQ ID NO: 59 or a fragment thereof. In some embodiments, the polypeptide of SEQ ID NO: 59, or a fragment thereof, is encoded by the polynucleotide of SEQ ID NO: 60 or a fragment thereof.

The methods can comprise evaluating the presence of the prostate cancer neoantigen comprising the amino acid sequence of SEQ ID NO: 61 or a fragment thereof. In some embodiments, the polypeptide of SEQ ID NO: 61, or a fragment thereof, is encoded by the polynucleotide of SEQ ID NO: 62 or a fragment thereof.

The methods can comprise evaluating the presence of the prostate cancer neoantigen comprising the amino acid sequence of SEQ ID NO: 63 or a fragment thereof. In some embodiments, the polypeptide of SEQ ID NO: 63, or a fragment thereof, is encoded by the polynucleotide of SEQ ID NO: 64 or a fragment thereof.

The methods can comprise evaluating the presence of the prostate cancer neoantigen comprising the amino acid sequence of SEQ ID NO: 65 or a fragment thereof. In some embodiments, the polypeptide of SEQ ID NO: 65, or a fragment thereof, is encoded by the polynucleotide of SEQ ID NO: 66 or a fragment thereof.

The methods can comprise evaluating the presence of the prostate cancer neoantigen comprising the amino acid sequence of SEQ ID NO: 67 or a fragment thereof. In some embodiments, the polypeptide of SEQ ID NO: 67, or a fragment thereof, is encoded by the polynucleotide of SEQ ID NO: 68 or a fragment thereof.

The methods can comprise evaluating the presence of the prostate cancer neoantigen comprising the amino acid sequence of SEQ ID NO: 69 or a fragment thereof. In some embodiments, the polypeptide of SEQ ID NO: 69, or a fragment thereof, is encoded by the polynucleotide of SEQ ID NO: 70 or a fragment thereof.

The methods can comprise evaluating the presence of the prostate cancer neoantigen comprising the amino acid sequence of SEQ ID NO: 71 or a fragment thereof. In some embodiments, the polypeptide of SEQ ID NO: 71, or a fragment thereof, is encoded by the polynucleotide of SEQ ID NO: 72 or a fragment thereof.

The methods can comprise evaluating the presence of the prostate cancer neoantigen comprising the amino acid sequence of SEQ ID NO: 73 or a fragment thereof. In some embodiments, the polypeptide of SEQ ID NO: 73, or a fragment thereof, is encoded by the polynucleotide of SEQ ID NO: 74 or a fragment thereof.

The methods can comprise evaluating the presence of the prostate cancer neoantigen comprising the amino acid sequence of SEQ ID NO: 75 or a fragment thereof. In some embodiments, the polypeptide of SEQ ID NO: 75, or a fragment thereof, is encoded by the polynucleotide of SEQ ID NO: 76 or a fragment thereof.

The methods can comprise evaluating the presence of the prostate cancer neoantigen comprising the amino acid sequence of SEQ ID NO: 77 or a fragment thereof. In some embodiments, the polypeptide of SEQ ID NO: 77, or a fragment thereof, is encoded by the polynucleotide of SEQ ID NO: 78 or a fragment thereof.

The methods can comprise evaluating the presence of the prostate cancer neoantigen comprising the amino acid sequence of SEQ ID NO: 79 or a fragment thereof. In some embodiments, the polypeptide of SEQ ID NO: 79, or a fragment thereof, is encoded by the polynucleotide of SEQ ID NO: 80 or a fragment thereof.

The methods can comprise evaluating the presence of the prostate cancer neoantigen comprising the amino acid sequence of SEQ ID NO: 81 or a fragment thereof. In some embodiments, the polypeptide of SEQ ID NO: 81, or a fragment thereof, is encoded by the polynucleotide of SEQ ID NO: 82 or a fragment thereof.

The methods can comprise evaluating the presence of the prostate cancer neoantigen comprising the amino acid sequence of SEQ ID NO: 83 or a fragment thereof. In some embodiments, the polypeptide of SEQ ID NO: 83, or a fragment thereof, is encoded by the polynucleotide of SEQ ID NO: 84 or a fragment thereof.

The methods can comprise evaluating the presence of the prostate cancer neoantigen comprising the amino acid sequence of SEQ ID NO: 85 or a fragment thereof. In some embodiments, the polypeptide of SEQ ID NO: 85, or a fragment thereof, is encoded by the polynucleotide of SEQ ID NO: 86 or a fragment thereof.

The methods can comprise evaluating the presence of the prostate cancer neoantigen comprising the amino acid sequence of SEQ ID NO: 87 or a fragment thereof. In some embodiments, the polypeptide of SEQ ID NO: 87, or a fragment thereof, is encoded by the polynucleotide of SEQ ID NO: 88 or a fragment thereof.

The methods can comprise evaluating the presence of the prostate cancer neoantigen comprising the amino acid sequence of SEQ ID NO: 89 or a fragment thereof. In some embodiments, the polypeptide of SEQ ID NO: 89, or a fragment thereof, is encoded by the polynucleotide of SEQ ID NO: 90 or a fragment thereof.

The methods can comprise evaluating the presence of the prostate cancer neoantigen comprising the amino acid sequence of SEQ ID NO: 91 or a fragment thereof. In some embodiments, the polypeptide of SEQ ID NO: 91, or a fragment thereof, is encoded by the polynucleotide of SEQ ID NO: 92 or a fragment thereof.

The methods can comprise evaluating the presence of the prostate cancer neoantigen comprising the amino acid sequence of SEQ ID NO: 93 or a fragment thereof. In some embodiments, the polypeptide of SEQ ID NO: 93, or a fragment thereof, is encoded by the polynucleotide of SEQ ID NO: 94 or a fragment thereof.

The methods can comprise evaluating the presence of the prostate cancer neoantigen comprising the amino acid sequence of SEQ ID NO: 95 or a fragment thereof. In some embodiments, the polypeptide of SEQ ID NO: 95, or a fragment thereof, is encoded by the polynucleotide of SEQ ID NO: 96 or a fragment thereof.

The methods can comprise evaluating the presence of the prostate cancer neoantigen comprising the amino acid sequence of SEQ ID NO: 97 or a fragment thereof. In some embodiments, the polypeptide of SEQ ID NO: 97, or a fragment thereof, is encoded by the polynucleotide of SEQ ID NO: 98 or a fragment thereof.

The methods can comprise evaluating the presence of the prostate cancer neoantigen comprising the amino acid sequence of SEQ ID NO: 99 or a fragment thereof. In some embodiments, the polypeptide of SEQ ID NO: 99, or a fragment thereof, is encoded by the polynucleotide of SEQ ID NO: 100 or a fragment thereof.

The methods can comprise evaluating the presence of the prostate cancer neoantigen comprising the amino acid sequence of SEQ ID NO: 101 or a fragment thereof. In some embodiments, the polypeptide of SEQ ID NO: 101, or a fragment thereof, is encoded by the polynucleotide of SEQ ID NO: 102 or a fragment thereof.

The methods can comprise evaluating the presence of the prostate cancer neoantigen comprising the amino acid sequence of SEQ ID NO: 103 or a fragment thereof. In some embodiments, the polypeptide of SEQ ID NO: 103, or a fragment thereof, is encoded by the polynucleotide of SEQ ID NO: 104 or a fragment thereof.

The methods can comprise evaluating the presence of the prostate cancer neoantigen comprising the amino acid sequence of SEQ ID NO: 105 or a fragment thereof. In some embodiments, the polypeptide of SEQ ID NO: 105, or a fragment thereof, is encoded by the polynucleotide of SEQ ID NO: 106 or a fragment thereof.

The methods can comprise evaluating the presence of the prostate cancer neoantigen comprising the amino acid sequence of SEQ ID NO: 107 or a fragment thereof. In some embodiments, the polypeptide of SEQ ID NO: 107, or a fragment thereof, is encoded by the polynucleotide of SEQ ID NO: 108 or a fragment thereof.

The methods can comprise evaluating the presence of the prostate cancer neoantigen comprising the amino acid sequence of SEQ ID NO: 109 or a fragment thereof. In some embodiments, the polypeptide of SEQ ID NO: 109, or a fragment thereof, is encoded by the polynucleotide of SEQ ID NO: 110 or a fragment thereof.

The methods can comprise evaluating the presence of the prostate cancer neoantigen comprising the amino acid sequence of SEQ ID NO: 111 or a fragment thereof. In some embodiments, the polypeptide of SEQ ID NO: 111, or a fragment thereof, is encoded by the polynucleotide of SEQ ID NO: 112 or a fragment thereof.

The methods can comprise evaluating the presence of the prostate cancer neoantigen comprising the amino acid sequence of SEQ ID NO: 113 or a fragment thereof. In some embodiments, the polypeptide of SEQ ID NO: 113, or a fragment thereof, is encoded by the polynucleotide of SEQ ID NO: 114 or a fragment thereof.

The methods can comprise evaluating the presence of the prostate cancer neoantigen comprising the amino acid sequence of SEQ ID NO: 115 or a fragment thereof. In some embodiments, the polypeptide of SEQ ID NO: 115, or a fragment thereof, is encoded by the polynucleotide of SEQ ID NO: 116 or a fragment thereof.

The methods can comprise evaluating the presence of the prostate cancer neoantigen comprising the amino acid sequence of SEQ ID NO: 117 or a fragment thereof. In some embodiments, the polypeptide of SEQ ID NO: 117, or a fragment thereof, is encoded by the polynucleotide of SEQ ID NO: 118 or a fragment thereof.

The methods can comprise evaluating the presence of the prostate cancer neoantigen comprising the amino acid sequence of SEQ ID NO: 119 or a fragment thereof. In some embodiments, the polypeptide of SEQ ID NO: 119, or a fragment thereof, is encoded by the polynucleotide of SEQ ID NO: 120 or a fragment thereof.

The methods can comprise evaluating the presence of the prostate cancer neoantigen comprising the amino acid sequence of SEQ ID NO: 121 or a fragment thereof. In some embodiments, the polypeptide of SEQ ID NO: 121, or a fragment thereof, is encoded by the polynucleotide of SEQ ID NO: 122 or a fragment thereof.

The methods can comprise evaluating the presence of the prostate cancer neoantigen comprising the amino acid sequence of SEQ ID NO: 123 or a fragment thereof. In some embodiments, the polypeptide of SEQ ID NO: 123, or a fragment thereof, is encoded by the polynucleotide of SEQ ID NO: 124 or a fragment thereof.

The methods can comprise evaluating the presence of the prostate cancer neoantigen comprising the amino acid sequence of SEQ ID NO: 125 or a fragment thereof. In some embodiments, the polypeptide of SEQ ID NO: 125, or a fragment thereof, is encoded by the polynucleotide of SEQ ID NO: 126 or a fragment thereof.

The methods can comprise evaluating the presence of the prostate cancer neoantigen comprising the amino acid sequence of SEQ ID NO: 127 or a fragment thereof. In some embodiments, the polypeptide of SEQ ID NO: 127, or a fragment thereof, is encoded by the polynucleotide of SEQ ID NO: 128 or a fragment thereof.

The methods can comprise evaluating the presence of the prostate cancer neoantigen comprising the amino acid sequence of SEQ ID NO: 129 or a fragment thereof. In some embodiments, the polypeptide of SEQ ID NO: 129, or a fragment thereof, is encoded by the polynucleotide of SEQ ID NO: 130 or a fragment thereof.

The methods can comprise evaluating the presence of the prostate cancer neoantigen comprising the amino acid sequence of SEQ ID NO: 131 or a fragment thereof. In some embodiments, the polypeptide of SEQ ID NO: 131, or a fragment thereof, is encoded by the polynucleotide of SEQ ID NO: 132 or a fragment thereof.

The methods can comprise evaluating the presence of the prostate cancer neoantigen comprising the amino acid sequence of SEQ ID NO: 133 or a fragment thereof. In some embodiments, the polypeptide of SEQ ID NO: 133, or a fragment thereof, is encoded by the polynucleotide of SEQ ID NO: 134 or a fragment thereof.

The methods can comprise evaluating the presence of the prostate cancer neoantigen comprising the amino acid sequence of SEQ ID NO: 135 or a fragment thereof. In some embodiments, the polypeptide of SEQ ID NO: 135, or a fragment thereof, is encoded by the polynucleotide of SEQ ID NO: 136 or a fragment thereof.

The methods can comprise evaluating the presence of the prostate cancer neoantigen comprising the amino acid sequence of SEQ ID NO: 137 or a fragment thereof. In some embodiments, the polypeptide of SEQ ID NO: 137, or a fragment thereof, is encoded by the polynucleotide of SEQ ID NO: 138 or a fragment thereof.

The methods can comprise evaluating the presence of the prostate cancer neoantigen comprising the amino acid sequence of SEQ ID NO: 139 or a fragment thereof. In some embodiments, the polypeptide of SEQ ID NO: 139, or a fragment thereof, is encoded by the polynucleotide of SEQ ID NO: 140 or a fragment thereof.

The methods can comprise evaluating the presence of the prostate cancer neoantigen comprising the amino acid sequence of SEQ ID NO: 141 or a fragment thereof. In some embodiments, the polypeptide of SEQ ID NO: 141, or a fragment thereof, is encoded by the polynucleotide of SEQ ID NO: 142 or a fragment thereof.

The methods can comprise evaluating the presence of the prostate cancer neoantigen comprising the amino acid sequence of SEQ ID NO: 143 or a fragment thereof. In some embodiments, the polypeptide of SEQ ID NO: 143, or a fragment thereof, is encoded by the polynucleotide of SEQ ID NO: 144 or a fragment thereof.

The methods can comprise evaluating the presence of the prostate cancer neoantigen comprising the amino acid sequence of SEQ ID NO: 145 or a fragment thereof. In some embodiments, the polypeptide of SEQ ID NO: 145, or a fragment thereof, is encoded by the polynucleotide of SEQ ID NO: 146 or a fragment thereof.

The methods can comprise evaluating the presence of the prostate cancer neoantigen comprising the amino acid sequence of SEQ ID NO: 147 or a fragment thereof. In some embodiments, the polypeptide of SEQ ID NO: 147, or a fragment thereof, is encoded by the polynucleotide of SEQ ID NO: 148 or a fragment thereof.

The methods can comprise evaluating the presence of the prostate cancer neoantigen comprising the amino acid sequence of SEQ ID NO: 149 or a fragment thereof. In some embodiments, the polypeptide of SEQ ID NO: 149, or a fragment thereof, is encoded by the polynucleotide of SEQ ID NO: 150 or a fragment thereof.

The methods can comprise evaluating the presence of the prostate cancer neoantigen comprising the amino acid sequence of SEQ ID NO: 151 or a fragment thereof. In some embodiments, the polypeptide of SEQ ID NO: 151, or a fragment thereof, is encoded by the polynucleotide of SEQ ID NO: 152 or a fragment thereof.

The methods can comprise evaluating the presence of the prostate cancer neoantigen comprising the amino acid sequence of SEQ ID NO: 153 or a fragment thereof. In some embodiments, the polypeptide of SEQ ID NO: 153, or a fragment thereof, is encoded by the polynucleotide of SEQ ID NO: 154 or a fragment thereof.

The methods can comprise evaluating the presence of the prostate cancer neoantigen comprising the amino acid sequence of SEQ ID NO: 155 or a fragment thereof. In some embodiments, the polypeptide of SEQ ID NO: 155, or a fragment thereof, is encoded by the polynucleotide of SEQ ID NO: 156 or a fragment thereof.

The methods can comprise evaluating the presence of the prostate cancer neoantigen comprising the amino acid sequence of SEQ ID NO: 157 or a fragment thereof. In some embodiments, the polypeptide of SEQ ID NO: 157, or a fragment thereof, is encoded by the polynucleotide of SEQ ID NO: 158 or a fragment thereof.

The methods can comprise evaluating the presence of the prostate cancer neoantigen comprising the amino acid sequence of SEQ ID NO: 159 or a fragment thereof. In some embodiments, the polypeptide of SEQ ID NO: 159, or a fragment thereof, is encoded by the polynucleotide of SEQ ID NO: 160 or a fragment thereof.

The methods can comprise evaluating the presence of the prostate cancer neoantigen comprising the amino acid sequence of SEQ ID NO: 161 or a fragment thereof. In some embodiments, the polypeptide of SEQ ID NO: 161, or a fragment thereof, is encoded by the polynucleotide of SEQ ID NO: 162 or a fragment thereof.

The methods can comprise evaluating the presence of the prostate cancer neoantigen comprising the amino acid sequence of SEQ ID NO: 163 or a fragment thereof. In some embodiments, the polypeptide of SEQ ID NO: 163, or a fragment thereof, is encoded by the polynucleotide of SEQ ID NO: 164 or a fragment thereof.

The methods can comprise evaluating the presence of the prostate cancer neoantigen comprising the amino acid sequence of SEQ ID NO: 165 or a fragment thereof. In some embodiments, the polypeptide of SEQ ID NO: 165, or a fragment thereof, is encoded by the polynucleotide of SEQ ID NO: 166 or a fragment thereof.

The methods can comprise evaluating the presence of the prostate cancer neoantigen comprising the amino acid sequence of SEQ ID NO: 167 or a fragment thereof. In some embodiments, the polypeptide of SEQ ID NO: 167, or a fragment thereof, is encoded by the polynucleotide of SEQ ID NO: 168 or a fragment thereof. In some embodiments, the polypeptide of SEQ ID NO: 167, or a fragment thereof, is encoded by the polynucleotide of SEQ ID NO: 495 or a fragment thereof. In some embodiments, the polypeptide of SEQ ID NO: 167, or a fragment thereof, is encoded by the polynucleotide of SEQ ID NO: 536 or a fragment thereof.

The methods can comprise evaluating the presence of the prostate cancer neoantigen comprising the amino acid sequence of SEQ ID NO: 169 or a fragment thereof. In some embodiments, the polypeptide of SEQ ID NO: 169, or a fragment thereof, is encoded by the polynucleotide of SEQ ID NO: 170 or a fragment thereof.

The methods can comprise evaluating the presence of the prostate cancer neoantigen comprising the amino acid sequence of SEQ ID NO: 171 or a fragment thereof. In some embodiments, the polypeptide of SEQ ID NO: 171, or a fragment thereof, is encoded by the polynucleotide of SEQ ID NO: 172 or a fragment thereof. In some embodiments, the polypeptide of SEQ ID NO: 171, or a fragment thereof, is encoded by the polynucleotide of SEQ ID NO: 496 or a fragment thereof. In some embodiments, the polypeptide of SEQ ID NO: 171, or a fragment thereof, is encoded by the polynucleotide of SEQ ID NO: 537 or a fragment thereof.

The methods can comprise evaluating the presence of the prostate cancer neoantigen comprising the amino acid sequence of SEQ ID NO: 173 or a fragment thereof. In some embodiments, the polypeptide of SEQ ID NO: 173, or a fragment thereof, is encoded by the polynucleotide of SEQ ID NO: 174 or a fragment thereof.

The methods can comprise evaluating the presence of the prostate cancer neoantigen comprising the amino acid sequence of SEQ ID NO: 175 or a fragment thereof. In some embodiments, the polypeptide of SEQ ID NO: 175, or a fragment thereof, is encoded by the polynucleotide of SEQ ID NO: 176 or a fragment thereof.

The methods can comprise evaluating the presence of the prostate cancer neoantigen comprising the amino acid sequence of SEQ ID NO: 177 or a fragment thereof. In some embodiments, the polypeptide of SEQ ID NO: 177, or a fragment thereof, is encoded by the polynucleotide of SEQ ID NO: 178 or a fragment thereof. In some embodiments, the polypeptide of SEQ ID NO: 177, or a fragment thereof, is encoded by the polynucleotide of SEQ ID NO: 499 or a fragment thereof. In some embodiments, the polypeptide of SEQ ID NO: 177, or a fragment thereof, is encoded by the polynucleotide of SEQ ID NO: 540 or a fragment thereof.

The methods can comprise evaluating the presence of the prostate cancer neoantigen comprising the amino acid sequence of SEQ ID NO: 179 or a fragment thereof. In some embodiments, the polypeptide of SEQ ID NO: 179, or a fragment thereof, is encoded by the polynucleotide of SEQ ID NO: 180 or a fragment thereof.

The methods can comprise evaluating the presence of the prostate cancer neoantigen comprising the amino acid sequence of SEQ ID NO: 181 or a fragment thereof. In some embodiments, the polypeptide of SEQ ID NO: 181, or a fragment thereof, is encoded by the polynucleotide of SEQ ID NO: 182 or a fragment thereof.

The methods can comprise evaluating the presence of the prostate cancer neoantigen comprising the amino acid sequence of SEQ ID NO: 183 or a fragment thereof. In some embodiments, the polypeptide of SEQ ID NO: 183, or a fragment thereof, is encoded by the polynucleotide of SEQ ID NO: 184 or a fragment thereof.

The methods can comprise evaluating the presence of the prostate cancer neoantigen comprising the amino acid sequence of SEQ ID NO: 185 or a fragment thereof. In some embodiments, the polypeptide of SEQ ID NO: 185, or a fragment thereof, is encoded by the polynucleotide of SEQ ID NO: 186 or a fragment thereof.

The methods can comprise evaluating the presence of the prostate cancer neoantigen comprising the amino acid sequence of SEQ ID NO: 187 or a fragment thereof. In some embodiments, the polypeptide of SEQ ID NO: 187, or a fragment thereof, is encoded by the polynucleotide of SEQ ID NO: 188 or a fragment thereof.

The methods can comprise evaluating the presence of the prostate cancer neoantigen comprising the amino acid sequence of SEQ ID NO: 189 or a fragment thereof. In some embodiments, the polypeptide of SEQ ID NO: 189, or a fragment thereof, is encoded by the polynucleotide of SEQ ID NO: 190 or a fragment thereof.

The methods can comprise evaluating the presence of the prostate cancer neoantigen comprising the amino acid sequence of SEQ ID NO: 191 or a fragment thereof. In some embodiments, the polypeptide of SEQ ID NO: 191, or a fragment thereof, is encoded by the polynucleotide of SEQ ID NO: 192 or a fragment thereof.

The methods can comprise evaluating the presence of the prostate cancer neoantigen comprising the amino acid sequence of SEQ ID NO: 193 or a fragment thereof. In some embodiments, the polypeptide of SEQ ID NO: 193, or a fragment thereof, is encoded by the polynucleotide of SEQ ID NO: 194 or a fragment thereof.

The methods can comprise evaluating the presence of the prostate cancer neoantigen comprising the amino acid sequence of SEQ ID NO: 195 or a fragment thereof. In some embodiments, the polypeptide of SEQ ID NO: 195, or a fragment thereof, is encoded by the polynucleotide of SEQ ID NO: 196 or a fragment thereof.

The methods can comprise evaluating the presence of the prostate cancer neoantigen comprising the amino acid sequence of SEQ ID NO: 197 or a fragment thereof. In some embodiments, the polypeptide of SEQ ID NO: 197, or a fragment thereof, is encoded by the polynucleotide of SEQ ID NO: 198 or a fragment thereof.

The methods can comprise evaluating the presence of the prostate cancer neoantigen comprising the amino acid sequence of SEQ ID NO: 199 or a fragment thereof. In some embodiments, the polypeptide of SEQ ID NO: 199, or a fragment thereof, is encoded by the polynucleotide of SEQ ID NO: 200 or a fragment thereof.

The methods can comprise evaluating the presence of the prostate cancer neoantigen comprising the amino acid sequence of SEQ ID NO: 201 or a fragment thereof. In some embodiments, the polypeptide of SEQ ID NO: 201, or a fragment thereof, is encoded by the polynucleotide of SEQ ID NO: 202 or a fragment thereof.

The methods can comprise evaluating the presence of the prostate cancer neoantigen comprising the amino acid sequence of SEQ ID NO: 203 or a fragment thereof. In some embodiments, the polypeptide of SEQ ID NO: 203, or a fragment thereof, is encoded by the polynucleotide of SEQ ID NO: 204 or a fragment thereof.

The methods can comprise evaluating the presence of the prostate cancer neoantigen comprising the amino acid sequence of SEQ ID NO: 205 or a fragment thereof. In some embodiments, the polypeptide of SEQ ID NO: 205, or a fragment thereof, is encoded by the polynucleotide of SEQ ID NO: 206 or a fragment thereof.

The methods can comprise evaluating the presence of the prostate cancer neoantigen comprising the amino acid sequence of SEQ ID NO: 207 or a fragment thereof. In some embodiments, the polypeptide of SEQ ID NO: 207, or a fragment thereof, is encoded by the polynucleotide of SEQ ID NO: 208 or a fragment thereof.

The methods can comprise evaluating the presence of the prostate cancer neoantigen comprising the amino acid sequence of SEQ ID NO: 209 or a fragment thereof. In some embodiments, the polypeptide of SEQ ID NO: 209, or a fragment thereof, is encoded by the polynucleotide of SEQ ID NO: 210 or a fragment thereof.

The methods can comprise evaluating the presence of the prostate cancer neoantigen comprising the amino acid sequence of SEQ ID NO: 211 or a fragment thereof. In some embodiments, the polypeptide of SEQ ID NO: 211, or a fragment thereof, is encoded by the polynucleotide of SEQ ID NO: 212 or a fragment thereof. In some embodiments, the polypeptide of SEQ ID NO: 211, or a fragment thereof, is encoded by the polynucleotide of SEQ ID NO: 484 or a fragment thereof. In some embodiments, the polypeptide of SEQ ID NO: 211, or a fragment thereof, is encoded by the polynucleotide of SEQ ID NO: 525 or a fragment thereof.

The methods can comprise evaluating the presence of the prostate cancer neoantigen comprising the amino acid sequence of SEQ ID NO: 213 or a fragment thereof. In some embodiments, the polypeptide of SEQ ID NO: 213, or a fragment thereof, is encoded by the polynucleotide of SEQ ID NO: 214 or a fragment thereof. In some embodiments, the polypeptide of SEQ ID NO: 213, or a fragment thereof, is encoded by the polynucleotide of SEQ ID NO: 486 or a fragment thereof.

The methods can comprise evaluating the presence of the prostate cancer neoantigen comprising the amino acid sequence of SEQ ID NO: 215 or a fragment thereof. In some embodiments, the polypeptide of SEQ ID NO: 215, or a fragment thereof, is encoded by the polynucleotide of SEQ ID NO: 216 or a fragment thereof. In some embodiments, the polypeptide of SEQ ID NO: 215, or a fragment thereof, is encoded by the polynucleotide of SEQ ID NO: 487 or a fragment thereof. In some embodiments, the polypeptide of SEQ ID NO: 215, or a fragment thereof, is encoded by the polynucleotide of SEQ ID NO: 528 or a fragment thereof.

The methods can comprise evaluating the presence of the prostate cancer neoantigen comprising the amino acid sequence of SEQ ID NO: 217 or a fragment thereof. In some embodiments, the polypeptide of SEQ ID NO: 217, or a fragment thereof, is encoded by the polynucleotide of SEQ ID NO: 218 or a fragment thereof.

The methods can comprise evaluating the presence of the prostate cancer neoantigen comprising the amino acid sequence of SEQ ID NO: 219 or a fragment thereof. In some embodiments, the polypeptide of SEQ ID NO: 219, or a fragment thereof, is encoded by the polynucleotide of SEQ ID NO: 220 or a fragment thereof. In some embodiments, the polypeptide of SEQ ID NO: 219, or a fragment thereof, is encoded by the polynucleotide of SEQ ID NO: 489 or a fragment thereof. In some embodiments, the polypeptide of SEQ ID NO: 219, or a fragment thereof, is encoded by the polynucleotide of SEQ ID NO: 530 or a fragment thereof.

The methods can comprise evaluating the presence of the prostate cancer neoantigen comprising the amino acid sequence of SEQ ID NO: 221 or a fragment thereof. In some embodiments, the polypeptide of SEQ ID NO: 221, or a fragment thereof, is encoded by the polynucleotide of SEQ ID NO: 222 or a fragment thereof. In some embodiments, the polypeptide of SEQ ID NO: 221, or a fragment thereof, is encoded by the polynucleotide of SEQ ID NO: 488 or a fragment thereof. In some embodiments, the polypeptide of SEQ ID NO: 221, or a fragment thereof, is encoded by the polynucleotide of SEQ ID NO: 529 or a fragment thereof.

The methods can comprise evaluating the presence of the prostate cancer neoantigen comprising the amino acid sequence of SEQ ID NO: 223 or a fragment thereof. In some embodiments, the polypeptide of SEQ ID NO: 223, or a fragment thereof, is encoded by the polynucleotide of SEQ ID NO: 224 or a fragment thereof. In some embodiments, the polypeptide of SEQ ID NO: 223, or a fragment thereof, is encoded by the polynucleotide of SEQ ID NO: 494 or a fragment thereof. In some embodiments, the polypeptide of SEQ ID NO: 223, or a fragment thereof, is encoded by the polynucleotide of SEQ ID NO: 535 or a fragment thereof.

The methods can comprise evaluating the presence of the prostate cancer neoantigen comprising the amino acid sequence of SEQ ID NO: 225 or a fragment thereof. In some embodiments, the polypeptide of SEQ ID NO: 225, or a fragment thereof, is encoded by the polynucleotide of SEQ ID NO: 226 or a fragment thereof. In some embodiments, the polypeptide of SEQ ID NO: 225, or a fragment thereof, is encoded by the polynucleotide of SEQ ID NO: 490 or a fragment thereof. In some embodiments, the polypeptide of SEQ ID NO: 225, or a fragment thereof, is encoded by the polynucleotide of SEQ ID NO: 531 or a fragment thereof.

The methods can comprise evaluating the presence of the prostate cancer neoantigen comprising the amino acid sequence of SEQ ID NO: 227 or a fragment thereof. In some embodiments, the polypeptide of SEQ ID NO: 227, or a fragment thereof, is encoded by the polynucleotide of SEQ ID NO: 228 or a fragment thereof.

The methods can comprise evaluating the presence of the prostate cancer neoantigen comprising the amino acid sequence of SEQ ID NO: 229 or a fragment thereof. In some embodiments, the polypeptide of SEQ ID NO: 229, or a fragment thereof, is encoded by the polynucleotide of SEQ ID NO: 230 or a fragment thereof.

The methods can comprise evaluating the presence of the prostate cancer neoantigen comprising the amino acid sequence of SEQ ID NO: 231 or a fragment thereof. In some embodiments, the polypeptide of SEQ ID NO: 231, or a fragment thereof, is encoded by the polynucleotide of SEQ ID NO: 232 or a fragment thereof.

The methods can comprise evaluating the presence of the prostate cancer neoantigen comprising the amino acid sequence of SEQ ID NO: 233 or a fragment thereof. In some embodiments, the polypeptide of SEQ ID NO: 233, or a fragment thereof, is encoded by the polynucleotide of SEQ ID NO: 234 or a fragment thereof.

The methods can comprise evaluating the presence of the prostate cancer neoantigen comprising the amino acid sequence of SEQ ID NO: 235 or a fragment thereof. In some embodiments, the polypeptide of SEQ ID NO: 235, or a fragment thereof, is encoded by the polynucleotide of SEQ ID NO: 236 or a fragment thereof. In some embodiments, the polypeptide of SEQ ID NO: 235, or a fragment thereof, is encoded by the polynucleotide of SEQ ID NO: 493 or a fragment thereof. In some embodiments, the polypeptide of SEQ ID NO: 235, or a fragment thereof, is encoded by the polynucleotide of SEQ ID NO: 534 or a fragment thereof.

The methods can comprise evaluating the presence of the prostate cancer neoantigen comprising the amino acid sequence of SEQ ID NO: 237 or a fragment thereof. In some embodiments, the polypeptide of SEQ ID NO: 237, or a fragment thereof, is encoded by the polynucleotide of SEQ ID NO: 238 or a fragment thereof.

The methods can comprise evaluating the presence of the prostate cancer neoantigen comprising the amino acid sequence of SEQ ID NO: 239 or a fragment thereof. In some embodiments, the polypeptide of SEQ ID NO: 239, or a fragment thereof, is encoded by the polynucleotide of SEQ ID NO: 240 or a fragment thereof.

The methods can comprise evaluating the presence of the prostate cancer neoantigen comprising the amino acid sequence of SEQ ID NO: 241 or a fragment thereof. In some embodiments, the polypeptide of SEQ ID NO: 241, or a fragment thereof, is encoded by the polynucleotide of SEQ ID NO: 242 or a fragment thereof.

The methods can comprise evaluating the presence of the prostate cancer neoantigen comprising the amino acid sequence of SEQ ID NO: 243 or a fragment thereof. In some embodiments, the polypeptide of SEQ ID NO: 243, or a fragment thereof, is encoded by the polynucleotide of SEQ ID NO: 244 or a fragment thereof.

The methods can comprise evaluating the presence of the prostate cancer neoantigen comprising the amino acid sequence of SEQ ID NO: 245 or a fragment thereof. In some embodiments, the polypeptide of SEQ ID NO: 245, or a fragment thereof, is encoded by the polynucleotide of SEQ ID NO: 246 or a fragment thereof. In some embodiments, the polypeptide of SEQ ID NO: 245, or a fragment thereof, is encoded by the polynucleotide of SEQ ID NO: 470 or a fragment thereof. In some embodiments, the polypeptide of SEQ ID NO: 245, or a fragment thereof, is encoded by the polynucleotide of SEQ ID NO: 511 or a fragment thereof.

The methods can comprise evaluating the presence of the prostate cancer neoantigen comprising the amino acid sequence of SEQ ID NO: 247 or a fragment thereof. In some embodiments, the polypeptide of SEQ ID NO: 247, or a fragment thereof, is encoded by the polynucleotide of SEQ ID NO: 248 or a fragment thereof.

The methods can comprise evaluating the presence of the prostate cancer neoantigen comprising the amino acid sequence of SEQ ID NO: 249 or a fragment thereof. In some embodiments, the polypeptide of SEQ ID NO: 249, or a fragment thereof, is encoded by the polynucleotide of SEQ ID NO: 250 or a fragment thereof.

The methods can comprise evaluating the presence of the prostate cancer neoantigen comprising the amino acid sequence of SEQ ID NO: 251 or a fragment thereof. In some embodiments, the polypeptide of SEQ ID NO: 251, or a fragment thereof, is encoded by the polynucleotide of SEQ ID NO: 252 or a fragment thereof. In some embodiments, the polypeptide of SEQ ID NO: 251, or a fragment thereof, is encoded by the polynucleotide of SEQ ID NO: 469 or a fragment thereof. In some embodiments, the polypeptide of SEQ ID NO: 251, or a fragment thereof, is encoded by the polynucleotide of SEQ ID NO: 510 or a fragment thereof.

The methods can comprise evaluating the presence of the prostate cancer neoantigen comprising the amino acid sequence of SEQ ID NO: 253 or a fragment thereof. In some embodiments, the polypeptide of SEQ ID NO: 253, or a fragment thereof, is encoded by the polynucleotide of SEQ ID NO: 254 or a fragment thereof. In some embodiments, the polypeptide of SEQ ID NO: 253, or a fragment thereof, is encoded by the polynucleotide of SEQ ID NO: 464 or a fragment thereof. In some embodiments, the polypeptide of SEQ ID NO: 253, or a fragment thereof, is encoded by the polynucleotide of SEQ ID NO: 505 or a fragment thereof.

The methods can comprise evaluating the presence of the prostate cancer neoantigen comprising the amino acid sequence of SEQ ID NO: 255 or a fragment thereof. In some embodiments, the polypeptide of SEQ ID NO: 255, or a fragment thereof, is encoded by the polynucleotide of SEQ ID NO: 256 or a fragment thereof. In some embodiments, the polypeptide of SEQ ID NO: 255, or a fragment thereof, is encoded by the polynucleotide of SEQ ID NO: 474 or a fragment thereof. In some embodiments, the polypeptide of SEQ ID NO: 255, or a fragment thereof, is encoded by the polynucleotide of SEQ ID NO: 515 or a fragment thereof.

The methods can comprise evaluating the presence of the prostate cancer neoantigen comprising the amino acid sequence of SEQ ID NO: 257 or a fragment thereof. In some embodiments, the polypeptide of SEQ ID NO: 257, or a fragment thereof, is encoded by the polynucleotide of SEQ ID NO: 258 or a fragment thereof.

The methods can comprise evaluating the presence of the prostate cancer neoantigen comprising the amino acid sequence of SEQ ID NO: 259 or a fragment thereof. In some embodiments, the polypeptide of SEQ ID NO: 259, or a fragment thereof, is encoded by the polynucleotide of SEQ ID NO: 260 or a fragment thereof.

The methods can comprise evaluating the presence of the prostate cancer neoantigen comprising the amino acid sequence of SEQ ID NO: 261 or a fragment thereof. In some embodiments, the polypeptide of SEQ ID NO: 261, or a fragment thereof, is encoded by the polynucleotide of SEQ ID NO: 262 or a fragment thereof. In some embodiments, the polypeptide of SEQ ID NO: 261, or a fragment thereof, is encoded by the polynucleotide of SEQ ID NO: 471 or a fragment thereof. In some embodiments, the polypeptide of SEQ ID NO: 261, or a fragment thereof, is encoded by the polynucleotide of SEQ ID NO: 512 or a fragment thereof.

The methods can comprise evaluating the presence of the prostate cancer neoantigen comprising the amino acid sequence of SEQ ID NO: 263 or a fragment thereof. In some embodiments, the polypeptide of SEQ ID NO: 263, or a fragment thereof, is encoded by the polynucleotide of SEQ ID NO: 264 or a fragment thereof.

The methods can comprise evaluating the presence of the prostate cancer neoantigen comprising the amino acid sequence of SEQ ID NO: 265 or a fragment thereof. In some embodiments, the polypeptide of SEQ ID NO: 265, or a fragment thereof, is encoded by the polynucleotide of SEQ ID NO: 266 or a fragment thereof. In some embodiments, the polypeptide of SEQ ID NO: 265, or a fragment thereof, is encoded by the polynucleotide of SEQ ID NO: 472 or a fragment thereof. In some embodiments, the polypeptide of SEQ ID NO: 265, or a fragment thereof, is encoded by the polynucleotide of SEQ ID NO: 513 or a fragment thereof.

The methods can comprise evaluating the presence of the prostate cancer neoantigen comprising the amino acid sequence of SEQ ID NO: 267 or a fragment thereof. In some embodiments, the polypeptide of SEQ ID NO: 267, or a fragment thereof, is encoded by the polynucleotide of SEQ ID NO: 268 or a fragment thereof.

The methods can comprise evaluating the presence of the prostate cancer neoantigen comprising the amino acid sequence of SEQ ID NO: 269 or a fragment thereof. In some embodiments, the polypeptide of SEQ ID NO: 269, or a fragment thereof, is encoded by the polynucleotide of SEQ ID NO: 270 or a fragment thereof. In some embodiments, the polypeptide of SEQ ID NO: 269, or a fragment thereof, is encoded by the polynucleotide of SEQ ID NO: 463 or a fragment thereof. In some embodiments, the polypeptide of SEQ ID NO: 269, or a fragment thereof, is encoded by the polynucleotide of SEQ ID NO: 504 or a fragment thereof.

The methods can comprise evaluating the presence of the prostate cancer neoantigen comprising the amino acid sequence of SEQ ID NO: 271 or a fragment thereof. In some embodiments, the polypeptide of SEQ ID NO: 271, or a fragment thereof, is encoded by the polynucleotide of SEQ ID NO: 272 or a fragment thereof. In some embodiments, the polypeptide of SEQ ID NO: 271, or a fragment thereof, is encoded by the polynucleotide of SEQ ID NO: 465 or a fragment thereof. In some embodiments, the polypeptide of SEQ ID NO: 271, or a fragment thereof, is encoded by the polynucleotide of SEQ ID NO: 508 or a fragment thereof.

The methods can comprise evaluating the presence of the prostate cancer neoantigen comprising the amino acid sequence of SEQ ID NO: 273 or a fragment thereof. In some embodiments, the polypeptide of SEQ ID NO: 273, or a fragment thereof, is encoded by the polynucleotide of SEQ ID NO: 274 or a fragment thereof.

The methods can comprise evaluating the presence of the prostate cancer neoantigen comprising the amino acid sequence of SEQ ID NO: 275 or a fragment thereof. In some embodiments, the polypeptide of SEQ ID NO: 275, or a fragment thereof, is encoded by the polynucleotide of SEQ ID NO: 276 or a fragment thereof. In some embodiments, the polypeptide of SEQ ID NO: 275, or a fragment thereof, is encoded by the polynucleotide of SEQ ID NO: 459 or a fragment thereof. In some embodiments, the polypeptide of SEQ ID NO: 275, or a fragment thereof, is encoded by the polynucleotide of SEQ ID NO: 500 or a fragment thereof.

The methods can comprise evaluating the presence of the prostate cancer neoantigen comprising the amino acid sequence of SEQ ID NO: 277 or a fragment thereof. In some embodiments, the polypeptide of SEQ ID NO: 277, or a fragment thereof, is encoded by the polynucleotide of SEQ ID NO: 278 or a fragment thereof. In some embodiments, the polypeptide of SEQ ID NO: 277, or a fragment thereof, is encoded by the polynucleotide of SEQ ID NO: 475 or a fragment thereof. In some embodiments, the polypeptide of SEQ ID NO: 277, or a fragment thereof, is encoded by the polynucleotide of SEQ ID NO: 516 or a fragment thereof.

The methods can comprise evaluating the presence of the prostate cancer neoantigen comprising the amino acid sequence of SEQ ID NO: 279 or a fragment thereof. In some embodiments, the polypeptide of SEQ ID NO: 279, or a fragment thereof, is encoded by the polynucleotide of SEQ ID NO: 280 or a fragment thereof.

The methods can comprise evaluating the presence of the prostate cancer neoantigen comprising the amino acid sequence of SEQ ID NO: 281 or a fragment thereof. In some embodiments, the polypeptide of SEQ ID NO: 281, or a fragment thereof, is encoded by the polynucleotide of SEQ ID NO: 282 or a fragment thereof.

The methods can comprise evaluating the presence of the prostate cancer neoantigen comprising the amino acid sequence of SEQ ID NO: 283 or a fragment thereof. In some embodiments, the polypeptide of SEQ ID NO: 283, or a fragment thereof, is encoded by the polynucleotide of SEQ ID NO: 284 or a fragment thereof.

The methods can comprise evaluating the presence of the prostate cancer neoantigen comprising the amino acid sequence of SEQ ID NO: 285 or a fragment thereof. In some embodiments, the polypeptide of SEQ ID NO: 285, or a fragment thereof, is encoded by the polynucleotide of SEQ ID NO: 286 or a fragment thereof. In some embodiments, the polypeptide of SEQ ID NO: 285, or a fragment thereof, is encoded by the polynucleotide of SEQ ID NO: 477 or a fragment thereof.

The methods can comprise evaluating the presence of the prostate cancer neoantigen comprising the amino acid sequence of SEQ ID NO: 287 or a fragment thereof. In some embodiments, the polypeptide of SEQ ID NO: 287, or a fragment thereof, is encoded by the polynucleotide of SEQ ID NO: 288 or a fragment thereof.

The methods can comprise evaluating the presence of the prostate cancer neoantigen comprising the amino acid sequence of SEQ ID NO: 289 or a fragment thereof. In some embodiments, the polypeptide of SEQ ID NO: 289, or a fragment thereof, is encoded by the polynucleotide of SEQ ID NO: 290 or a fragment thereof.

The methods can comprise evaluating the presence of the prostate cancer neoantigen comprising the amino acid sequence of SEQ ID NO: 291 or a fragment thereof. In some embodiments, the polypeptide of SEQ ID NO: 291, or a fragment thereof, is encoded by the polynucleotide of SEQ ID NO: 292 or a fragment thereof.

The methods can comprise evaluating the presence of the prostate cancer neoantigen comprising the amino acid sequence of SEQ ID NO: 293 or a fragment thereof. In some embodiments, the polypeptide of SEQ ID NO: 293, or a fragment thereof, is encoded by the polynucleotide of SEQ ID NO: 294 or a fragment thereof.

The methods can comprise evaluating the presence of the prostate cancer neoantigen comprising the amino acid sequence of SEQ ID NO: 295 or a fragment thereof. In some embodiments, the polypeptide of SEQ ID NO: 295, or a fragment thereof, is encoded by the polynucleotide of SEQ ID NO: 296 or a fragment thereof.

The methods can comprise evaluating the presence of the prostate cancer neoantigen comprising the amino acid sequence of SEQ ID NO: 297 or a fragment thereof. In some embodiments, the polypeptide of SEQ ID NO: 297, or a fragment thereof, is encoded by the polynucleotide of SEQ ID NO: 298 or a fragment thereof. In some embodiments, the polypeptide of SEQ ID NO: 297, or a fragment thereof, is encoded by the polynucleotide of SEQ ID NO: 476 or a fragment thereof. In some embodiments, the polypeptide of SEQ ID NO: 297, or a fragment thereof, is encoded by the polynucleotide of SEQ ID NO: 517 or a fragment thereof.

The methods can comprise evaluating the presence of the prostate cancer neoantigen comprising the amino acid sequence of SEQ ID NO: 299 or a fragment thereof. In some embodiments, the polypeptide of SEQ ID NO: 299, or a fragment thereof, is encoded by the polynucleotide of SEQ ID NO: 300 or a fragment thereof.

The methods can comprise evaluating the presence of the prostate cancer neoantigen comprising the amino acid sequence of SEQ ID NO: 301 or a fragment thereof. In some embodiments, the polypeptide of SEQ ID NO: 301, or a fragment thereof, is encoded by the polynucleotide of SEQ ID NO: 302 or a fragment thereof.

The methods can comprise evaluating the presence of the prostate cancer neoantigen comprising the amino acid sequence of SEQ ID NO: 303 or a fragment thereof. In some embodiments, the polypeptide of SEQ ID NO: 303, or a fragment thereof, is encoded by the polynucleotide of SEQ ID NO: 304 or a fragment thereof.

The methods can comprise evaluating the presence of the prostate cancer neoantigen comprising the amino acid sequence of SEQ ID NO: 305 or a fragment thereof. In some embodiments, the polypeptide of SEQ ID NO: 305, or a fragment thereof, is encoded by the polynucleotide of SEQ ID NO: 306 or a fragment thereof. In some embodiments, the polypeptide of SEQ ID NO: 305, or a fragment thereof, is encoded by the polynucleotide of SEQ ID NO: 468 or a fragment thereof. In some embodiments, the polypeptide of SEQ ID NO: 305, or a fragment thereof, is encoded by the polynucleotide of SEQ ID NO: 509 or a fragment thereof.

The methods can comprise evaluating the presence of the prostate cancer neoantigen comprising the amino acid sequence of SEQ ID NO: 307 or a fragment thereof. In some embodiments, the polypeptide of SEQ ID NO: 307, or a fragment thereof, is encoded by the polynucleotide of SEQ ID NO: 308 or a fragment thereof.

The methods can comprise evaluating the presence of the prostate cancer neoantigen comprising the amino acid sequence of SEQ ID NO: 309 or a fragment thereof. In some embodiments, the polypeptide of SEQ ID NO: 309, or a fragment thereof, is encoded by the polynucleotide of SEQ ID NO: 310 or a fragment thereof. In some embodiments, the polypeptide of SEQ ID NO: 309, or a fragment thereof, is encoded by the polynucleotide of SEQ ID NO: 465 or a fragment thereof. In some embodiments, the polypeptide of SEQ ID NO: 309, or a fragment thereof, is encoded by the polynucleotide of SEQ ID NO: 506 or a fragment thereof.

The methods can comprise evaluating the presence of the prostate cancer neoantigen comprising the amino acid sequence of SEQ ID NO: 311 or a fragment thereof. In some embodiments, the polypeptide of SEQ ID NO: 311, or a fragment thereof, is encoded by the polynucleotide of SEQ ID NO: 312 or a fragment thereof.

The methods can comprise evaluating the presence of the prostate cancer neoantigen comprising the amino acid sequence of SEQ ID NO: 313 or a fragment thereof. In some embodiments, the polypeptide of SEQ ID NO: 313, or a fragment thereof, is encoded by the polynucleotide of SEQ ID NO: 314 or a fragment thereof.

The methods can comprise evaluating the presence of the prostate cancer neoantigen comprising the amino acid sequence of SEQ ID NO: 315 or a fragment thereof. In some embodiments, the polypeptide of SEQ ID NO: 315, or a fragment thereof, is encoded by the polynucleotide of SEQ ID NO: 316 or a fragment thereof.

The methods can comprise evaluating the presence of the prostate cancer neoantigen comprising the amino acid sequence of SEQ ID NO: 317 or a fragment thereof. In some embodiments, the polypeptide of SEQ ID NO: 317, or a fragment thereof, is encoded by the polynucleotide of SEQ ID NO: 318 or a fragment thereof. In some embodiments, the polypeptide of SEQ ID NO: 317, or a fragment thereof, is encoded by the polynucleotide of SEQ ID NO: 473 or a fragment thereof. In some embodiments, the polypeptide of SEQ ID NO: 317, or a fragment thereof, is encoded by the polynucleotide of SEQ ID NO: 514 or a fragment thereof.

The methods can comprise evaluating the presence of the prostate cancer neoantigen comprising the amino acid sequence of SEQ ID NO: 319 or a fragment thereof. In some embodiments, the polypeptide of SEQ ID NO: 319, or a fragment thereof, is encoded by the polynucleotide of SEQ ID NO: 320 or a fragment thereof.

The methods can comprise evaluating the presence of the prostate cancer neoantigen comprising the amino acid sequence of SEQ ID NO: 321 or a fragment thereof. In some embodiments, the polypeptide of SEQ ID NO: 321, or a fragment thereof, is encoded by the polynucleotide of SEQ ID NO: 322 or a fragment thereof.

The methods can comprise evaluating the presence of the prostate cancer neoantigen comprising the amino acid sequence of SEQ ID NO: 323 or a fragment thereof. In some embodiments, the polypeptide of SEQ ID NO: 323, or a fragment thereof, is encoded by the polynucleotide of SEQ ID NO: 324 or a fragment thereof.

The methods can comprise evaluating the presence of the prostate cancer neoantigen comprising the amino acid sequence of SEQ ID NO: 325 or a fragment thereof. In some embodiments, the polypeptide of SEQ ID NO: 325, or a fragment thereof, is encoded by the polynucleotide of SEQ ID NO: 326 or a fragment thereof. In some embodiments, the polypeptide of SEQ ID NO: 325, or a fragment thereof, is encoded by the polynucleotide of SEQ ID NO: 466 or a fragment thereof. In some embodiments, the polypeptide of SEQ ID NO: 325, or a fragment thereof, is encoded by the polynucleotide of SEQ ID NO: 507 or a fragment thereof.

The methods can comprise evaluating the presence of the prostate cancer neoantigen comprising the amino acid sequence of SEQ ID NO: 327 or a fragment thereof. In some embodiments, the polypeptide of SEQ ID NO: 327, or a fragment thereof, is encoded by the polynucleotide of SEQ ID NO: 328 or a fragment thereof.

The methods can comprise evaluating the presence of the prostate cancer neoantigen comprising the amino acid sequence of SEQ ID NO: 329 or a fragment thereof. In some embodiments, the polypeptide of SEQ ID NO: 329, or a fragment thereof, is encoded by the polynucleotide of SEQ ID NO: 330 or a fragment thereof.

The methods can comprise evaluating the presence of the prostate cancer neoantigen comprising the amino acid sequence of SEQ ID NO: 331 or a fragment thereof. In some embodiments, the polypeptide of SEQ ID NO: 331, or a fragment thereof, is encoded by the polynucleotide of SEQ ID NO: 332 or a fragment thereof.

The methods can comprise evaluating the presence of the prostate cancer neoantigen comprising the amino acid sequence of SEQ ID NO: 333 or a fragment thereof. In some embodiments, the polypeptide of SEQ ID NO: 333, or a fragment thereof, is encoded by the polynucleotide of SEQ ID NO: 334 or a fragment thereof. In some embodiments, the polypeptide of SEQ ID NO: 333, or a fragment thereof, is encoded by the polynucleotide of SEQ ID NO: 461 or a fragment thereof.

The methods can comprise evaluating the presence of the prostate cancer neoantigen comprising the amino acid sequence of SEQ ID NO: 335 or a fragment thereof. In some embodiments, the polypeptide of SEQ ID NO: 335, or a fragment thereof, is encoded by the polynucleotide of SEQ ID NO: 336 or a fragment thereof.

The methods can comprise evaluating the presence of the prostate cancer neoantigen comprising the amino acid sequence of SEQ ID NO: 337 or a fragment thereof. In some embodiments, the polypeptide of SEQ ID NO: 337, or a fragment thereof, is encoded by the polynucleotide of SEQ ID NO: 338 or a fragment thereof. In some embodiments, the polypeptide of SEQ ID NO: 337, or a fragment thereof, is encoded by the polynucleotide of SEQ ID NO: 462 or a fragment thereof. In some embodiments, the polypeptide of SEQ ID NO: 337, or a fragment thereof, is encoded by the polynucleotide of SEQ ID NO: 503 or a fragment thereof.

The methods can comprise evaluating the presence of the prostate cancer neoantigen comprising the amino acid sequence of SEQ ID NO: 339 or a fragment thereof. In some embodiments, the polypeptide of SEQ ID NO: 339, or a fragment thereof, is encoded by the polynucleotide of SEQ ID NO: 340 or a fragment thereof.

The methods can comprise evaluating the presence of the prostate cancer neoantigen comprising the amino acid sequence of SEQ ID NO: 341 or a fragment thereof. In some embodiments, the polypeptide of SEQ ID NO: 341, or a fragment thereof, is encoded by the polynucleotide of SEQ ID NO: 342 or a fragment thereof.

The methods can comprise evaluating the presence of the prostate cancer neoantigen comprising the amino acid sequence of SEQ ID NO: 343 or a fragment thereof. In some embodiments, the polypeptide of SEQ ID NO: 343, or a fragment thereof, is encoded by the polynucleotide of SEQ ID NO: 344 or a fragment thereof. In some embodiments, the polypeptide of SEQ ID NO: 343, or a fragment thereof, is encoded by the polynucleotide of SEQ ID NO: 483 or a fragment thereof. In some embodiments, the polypeptide of SEQ ID NO: 343, or a fragment thereof, is encoded by the polynucleotide of SEQ ID NO: 524 or a fragment thereof.

The methods can comprise evaluating the presence of the prostate cancer neoantigen comprising the amino acid sequence of SEQ ID NO: 345 or a fragment thereof. In some embodiments, the polypeptide of SEQ ID NO: 345, or a fragment thereof, is encoded by the polynucleotide of SEQ ID NO: 346 or a fragment thereof. In some embodiments, the polypeptide of SEQ ID NO: 345, or a fragment thereof, is encoded by the polynucleotide of SEQ ID NO: 491 or a fragment thereof. In some embodiments, the polypeptide of SEQ ID NO: 345, or a fragment thereof, is encoded by the polynucleotide of SEQ ID NO: 532 or a fragment thereof.

The methods can comprise evaluating the presence of the prostate cancer neoantigen comprising the amino acid sequence of SEQ ID NO: 347 or a fragment thereof. In some embodiments, the polypeptide of SEQ ID NO: 347, or a fragment thereof, is encoded by the polynucleotide of SEQ ID NO: 348 or a fragment thereof.

The methods can comprise evaluating the presence of the prostate cancer neoantigen comprising the amino acid sequence of SEQ ID NO: 349 or a fragment thereof. In some embodiments, the polypeptide of SEQ ID NO: 349, or a fragment thereof, is encoded by the polynucleotide of SEQ ID NO: 350 or a fragment thereof. In some embodiments, the polypeptide of SEQ ID NO: 349, or a fragment thereof, is encoded by the polynucleotide of SEQ ID NO: 485 or a fragment thereof.

The methods can comprise evaluating the presence of the prostate cancer neoantigen comprising the amino acid sequence of SEQ ID NO: 351 or a fragment thereof. In some embodiments, the polypeptide of SEQ ID NO: 351, or a fragment thereof, is encoded by the polynucleotide of SEQ ID NO: 352 or a fragment thereof.

The methods can comprise evaluating the presence of the prostate cancer neoantigen comprising the amino acid sequence of SEQ ID NO: 353 or a fragment thereof. In some embodiments, the polypeptide of SEQ ID NO: 353, or a fragment thereof, is encoded by the polynucleotide of SEQ ID NO: 354 or a fragment thereof. In some embodiments, the polypeptide of SEQ ID NO: 353, or a fragment thereof, is encoded by the polynucleotide of SEQ ID NO: 492 or a fragment thereof. In some embodiments, the polypeptide of SEQ ID NO: 353, or a fragment thereof, is encoded by the polynucleotide of SEQ ID NO: 533 or a fragment thereof.

The methods can comprise evaluating the presence of the prostate cancer neoantigen comprising the amino acid sequence of SEQ ID NO: 355 or a fragment thereof. In some embodiments, the polypeptide of SEQ ID NO: 355, or a fragment thereof, is encoded by the polynucleotide of SEQ ID NO: 356 or a fragment thereof.

The methods can comprise evaluating the presence of the prostate cancer neoantigen comprising the amino acid sequence of SEQ ID NO: 357 or a fragment thereof. In some embodiments, the polypeptide of SEQ ID NO: 357, or a fragment thereof, is encoded by the polynucleotide of SEQ ID NO: 358 or a fragment thereof.

The methods can comprise evaluating the presence of the prostate cancer neoantigen comprising the amino acid sequence of SEQ ID NO: 359 or a fragment thereof. In some embodiments, the polypeptide of SEQ ID NO: 359, or a fragment thereof, is encoded by the polynucleotide of SEQ ID NO: 360 or a fragment thereof.

The methods can comprise evaluating the presence of the prostate cancer neoantigen comprising the amino acid sequence of SEQ ID NO: 361 or a fragment thereof. In some embodiments, the polypeptide of SEQ ID NO: 361, or a fragment thereof, is encoded by the polynucleotide of SEQ ID NO: 362 or a fragment thereof.

The methods can comprise evaluating the presence of the prostate cancer neoantigen comprising the amino acid sequence of SEQ ID NO: 363 or a fragment thereof. In some embodiments, the polypeptide of SEQ ID NO: 363, or a fragment thereof, is encoded by the polynucleotide of SEQ ID NO: 364 or a fragment thereof.

The methods can comprise evaluating the presence of the prostate cancer neoantigen comprising the amino acid sequence of SEQ ID NO: 365 or a fragment thereof. In some embodiments, the polypeptide of SEQ ID NO: 365, or a fragment thereof, is encoded by the polynucleotide of SEQ ID NO: 366 or a fragment thereof.

The methods can comprise evaluating the presence of the prostate cancer neoantigen comprising the amino acid sequence of SEQ ID NO: 367 or a fragment thereof. In some embodiments, the polypeptide of SEQ ID NO: 367, or a fragment thereof, is encoded by the polynucleotide of SEQ ID NO: 368 or a fragment thereof.

The methods can comprise evaluating the presence of the prostate cancer neoantigen comprising the amino acid sequence of SEQ ID NO: 369 or a fragment thereof. In some embodiments, the polypeptide of SEQ ID NO: 369, or a fragment thereof, is encoded by the polynucleotide of SEQ ID NO: 370 or a fragment thereof.

The methods can comprise evaluating the presence of the prostate cancer neoantigen comprising the amino acid sequence of SEQ ID NO: 371 or a fragment thereof. In some embodiments, the polypeptide of SEQ ID NO: 371, or a fragment thereof, is encoded by the polynucleotide of SEQ ID NO: 372 or a fragment thereof.

The methods can comprise evaluating the presence of the prostate cancer neoantigen comprising the amino acid sequence of SEQ ID NO: 373 or a fragment thereof. In some embodiments, the polypeptide of SEQ ID NO: 373, or a fragment thereof, is encoded by the polynucleotide of SEQ ID NO: 374 or a fragment thereof.

The methods can comprise evaluating the presence of the prostate cancer neoantigen comprising the amino acid sequence of SEQ ID NO: 375 or a fragment thereof. In some embodiments, the polypeptide of SEQ ID NO: 375, or a fragment thereof, is encoded by the polynucleotide of SEQ ID NO: 376 or a fragment thereof.

The methods can comprise evaluating the presence of the prostate cancer neoantigen comprising the amino acid sequence of SEQ ID NO: 379 or a fragment thereof. In some embodiments, the polypeptide of SEQ ID NO: 379, or a fragment thereof, is encoded by the polynucleotide of SEQ ID NO: 380 or a fragment thereof. In some embodiments, the polypeptide of SEQ ID NO: 379, or a fragment thereof, is encoded by the polynucleotide of SEQ ID NO: 482 or a fragment thereof. In some embodiments, the polypeptide of SEQ ID NO: 379, or a fragment thereof, is encoded by the polynucleotide of SEQ ID NO: 523 or a fragment thereof.

The methods can comprise evaluating the presence of the prostate cancer neoantigen comprising the amino acid sequence of SEQ ID NO: 381 or a fragment thereof. In some embodiments, the polypeptide of SEQ ID NO: 381, or a fragment thereof, is encoded by the polynucleotide of SEQ ID NO: 382 or a fragment thereof. In some embodiments, the polypeptide of SEQ ID NO: 381, or a fragment thereof, is encoded by the polynucleotide of SEQ ID NO: 460 or a fragment thereof. In some embodiments, the polypeptide of SEQ ID NO: 381, or a fragment thereof, is encoded by the polynucleotide of SEQ ID NO: 501 or a fragment thereof.

The methods can comprise evaluating the presence of the prostate cancer neoantigen comprising the amino acid sequence of SEQ ID NO: 383 or a fragment thereof. In some embodiments, the polypeptide of SEQ ID NO: 383, or a fragment thereof, is encoded by the polynucleotide of SEQ ID NO: 384 or a fragment thereof.

The methods can comprise evaluating the presence of the prostate cancer neoantigen comprising the amino acid sequence of SEQ ID NO: 385 or a fragment thereof. In some embodiments, the polypeptide of SEQ ID NO: 385, or a fragment thereof, is encoded by the polynucleotide of SEQ ID NO: 386 or a fragment thereof.

The methods can comprise evaluating the presence of the prostate cancer neoantigen comprising the amino acid sequence of SEQ ID NO: 387 or a fragment thereof.

The methods can comprise evaluating the presence of the prostate cancer neoantigen comprising the amino acid sequence of SEQ ID NO: 388 or a fragment thereof.

The methods can comprise evaluating the presence of the prostate cancer neoantigen comprising the amino acid sequence of SEQ ID NO: 389 or a fragment thereof.

The methods can comprise evaluating the presence of the prostate cancer neoantigen comprising the amino acid sequence of SEQ ID NO: 390 or a fragment thereof.

The methods can comprise evaluating the presence of the prostate cancer neoantigen comprising the amino acid sequence of SEQ ID NO: 391 or a fragment thereof.

The methods can comprise evaluating the presence of the prostate cancer neoantigen comprising the amino acid sequence of SEQ ID NO: 392 or a fragment thereof.

The methods can comprise evaluating the presence of the prostate cancer neoantigen comprising the amino acid sequence of SEQ ID NO: 393 or a fragment thereof.

The methods can comprise evaluating the presence of the prostate cancer neoantigen comprising the amino acid sequence of SEQ ID NO: 394 or a fragment thereof.

The methods can comprise evaluating the presence of the prostate cancer neoantigen comprising the amino acid sequence of SEQ ID NO: 395 or a fragment thereof.

The methods can comprise evaluating the presence of the prostate cancer neoantigen comprising the amino acid sequence of SEQ ID NO: 396 or a fragment thereof.

The methods can comprise evaluating the presence of the prostate cancer neoantigen comprising the amino acid sequence of SEQ ID NO: 397 or a fragment thereof.

The methods can comprise evaluating the presence of the prostate cancer neoantigen comprising the amino acid sequence of SEQ ID NO: 398 or a fragment thereof.

The methods can comprise evaluating the presence of the prostate cancer neoantigen comprising the amino acid sequence of SEQ ID NO: 399 or a fragment thereof.

The methods can comprise evaluating the presence of the prostate cancer neoantigen comprising the amino acid sequence of SEQ ID NO: 400 or a fragment thereof.

The methods can comprise evaluating the presence of the prostate cancer neoantigen comprising the amino acid sequence of SEQ ID NO: 401 or a fragment thereof.

The methods can comprise evaluating the presence of the prostate cancer neoantigen comprising the amino acid sequence of SEQ ID NO: 402 or a fragment thereof.

The methods can comprise evaluating the presence of the prostate cancer neoantigen comprising the amino acid sequence of SEQ ID NO: 403 or a fragment thereof.

The methods can comprise evaluating the presence of the prostate cancer neoantigen comprising the amino acid sequence of SEQ ID NO: 404 or a fragment thereof.

The methods can comprise evaluating the presence of the prostate cancer neoantigen comprising the amino acid sequence of SEQ ID NO: 405 or a fragment thereof.

The methods can comprise evaluating the presence of the prostate cancer neoantigen comprising the amino acid sequence of SEQ ID NO: 406 or a fragment thereof.

The methods can comprise evaluating the presence of the prostate cancer neoantigen comprising the amino acid sequence of SEQ ID NO: 407 or a fragment thereof.

The methods can comprise evaluating the presence of the prostate cancer neoantigen comprising the amino acid sequence of SEQ ID NO: 408 or a fragment thereof.

The methods can comprise evaluating the presence of the prostate cancer neoantigen comprising the amino acid sequence of SEQ ID NO: 426 or a fragment thereof.

The methods can comprise evaluating the presence of the prostate cancer neoantigen comprising the amino acid sequence of SEQ ID NO: 427 or a fragment thereof.

The methods can comprise evaluating the presence of the prostate cancer neoantigen comprising the amino acid sequence of SEQ ID NO: 428 or a fragment thereof.

The methods can comprise evaluating the presence of the prostate cancer neoantigen comprising the amino acid sequence of SEQ ID NO: 429 or a fragment thereof.

The methods can comprise evaluating the presence of the prostate cancer neoantigen comprising the amino acid sequence of SEQ ID NO: 430 or a fragment thereof.

The methods can comprise evaluating the presence of the prostate cancer neoantigen comprising the amino acid sequence of SEQ ID NO: 431 or a fragment thereof.

The methods can comprise evaluating the presence of the prostate cancer neoantigen comprising the amino acid sequence of SEQ ID NO: 432 or a fragment thereof.

The methods can comprise evaluating the presence of the prostate cancer neoantigen comprising the amino acid sequence of SEQ ID NO: 433 or a fragment thereof.

The methods can comprise evaluating the presence of the prostate cancer neoantigen comprising the amino acid sequence of SEQ ID NO: 434 or a fragment thereof.

The methods can comprise evaluating the presence of the prostate cancer neoantigen comprising the amino acid sequence of SEQ ID NO: 435 or a fragment thereof.

The methods can comprise evaluating the presence of the prostate cancer neoantigen comprising the amino acid sequence of SEQ ID NO: 436 or a fragment thereof.

The methods can comprise evaluating the presence of the prostate cancer neoantigen comprising the amino acid sequence of SEQ ID NO: 437 or a fragment thereof. In some embodiments, the polypeptide of SEQ ID NO: 437, or a fragment thereof, is encoded by the polynucleotide of SEQ ID NO: 448 or a fragment thereof. In some embodiments, the polypeptide of SEQ ID NO: 437, or a fragment thereof, is encoded by the polynucleotide of SEQ ID NO: 478 or a fragment thereof. In some embodiments, the polypeptide of SEQ ID NO: 437, or a fragment thereof, is encoded by the polynucleotide of SEQ ID NO: 519 or a fragment thereof.

The methods can comprise evaluating the presence of the prostate cancer neoantigen comprising the amino acid sequence of SEQ ID NO: 438 or a fragment thereof. In some embodiments, the polypeptide of SEQ ID NO: 438, or a fragment thereof, is encoded by the polynucleotide of SEQ ID NO: 449 or a fragment thereof.

The methods can comprise evaluating the presence of the prostate cancer neoantigen comprising the amino acid sequence of SEQ ID NO: 439 or a fragment thereof. In some embodiments, the polypeptide of SEQ ID NO: 439, or a fragment thereof, is encoded by the polynucleotide of SEQ ID NO: 450 or a fragment thereof. In some embodiments, the polypeptide of SEQ ID NO: 439, or a fragment thereof, is encoded by the polynucleotide of SEQ ID NO: 479 or a fragment thereof. In some embodiments, the polypeptide of SEQ ID NO: 439, or a fragment thereof, is encoded by the polynucleotide of SEQ ID NO: 520 or a fragment thereof.

The methods can comprise evaluating the presence of the prostate cancer neoantigen comprising the amino acid sequence of SEQ ID NO: 440 or a fragment thereof. In some embodiments, the polypeptide of SEQ ID NO: 440, or a fragment thereof, is encoded by the polynucleotide of SEQ ID NO: 451 or a fragment thereof.

The methods can comprise evaluating the presence of the prostate cancer neoantigen comprising the amino acid sequence of SEQ ID NO: 441 or a fragment thereof. In some embodiments, the polypeptide of SEQ ID NO: 441, or a fragment thereof, is encoded by the polynucleotide of SEQ ID NO: 452 or a fragment thereof.

The methods can comprise evaluating the presence of the prostate cancer neoantigen comprising the amino acid sequence of SEQ ID NO: 442 or a fragment thereof. In some embodiments, the polypeptide of SEQ ID NO: 442, or a fragment thereof, is encoded by the polynucleotide of SEQ ID NO: 453 or a fragment thereof. In some embodiments, the polypeptide of SEQ ID NO: 442, or a fragment thereof, is encoded by the polynucleotide of SEQ ID NO: 480 or a fragment thereof. In some embodiments, the polypeptide of SEQ ID NO: 442, or a fragment thereof, is encoded by the polynucleotide of SEQ ID NO: 521 or a fragment thereof.

The methods can comprise evaluating the presence of the prostate cancer neoantigen comprising the amino acid sequence of SEQ ID NO: 443 or a fragment thereof. In some embodiments, the polypeptide of SEQ ID NO: 443, or a fragment thereof, is encoded by the polynucleotide of SEQ ID NO: 454 or a fragment thereof.

The methods can comprise evaluating the presence of the prostate cancer neoantigen comprising the amino acid sequence of SEQ ID NO: 444 or a fragment thereof. In some embodiments, the polypeptide of SEQ ID NO: 444, or a fragment thereof, is encoded by the polynucleotide of SEQ ID NO: 455 or a fragment thereof. In some embodiments, the polypeptide of SEQ ID NO: 444, or a fragment thereof, is encoded by the polynucleotide of SEQ ID NO: 481 or a fragment thereof. In some embodiments, the polypeptide of SEQ ID NO: 444, or a fragment thereof, is encoded by the polynucleotide of SEQ ID NO: 522 or a fragment thereof.

The methods can comprise evaluating the presence of the prostate cancer neoantigen comprising the amino acid sequence of SEQ ID NO: 445 or a fragment thereof. In some embodiments, the polypeptide of SEQ ID NO: 445, or a fragment thereof, is encoded by the polynucleotide of SEQ ID NO: 456 or a fragment thereof.

The methods can comprise evaluating the presence of the prostate cancer neoantigen comprising the amino acid sequence of SEQ ID NO: 446 or a fragment thereof. In some embodiments, the polypeptide of SEQ ID NO: 446, or a fragment thereof, is encoded by the polynucleotide of SEQ ID NO: 457 or a fragment thereof.

The methods can comprise evaluating the presence of the prostate cancer neoantigen comprising the amino acid sequence of SEQ ID NO: 447 or a fragment thereof. In some embodiments, the polypeptide of SEQ ID NO: 447, or a fragment thereof, is encoded by the polynucleotide of SEQ ID NO: 458 or a fragment thereof.

The fragments of the prostate cancer neoantigens can comprise at least 6 amino acids. In some embodiments, the fragments comprise at least 7 amino acids In some embodiments, the fragments comprise at least 8 amino acids. In some embodiments, the fragments comprise at least 9 amino acids. In some embodiments, the fragments comprise at least 10 amino acids. In some embodiments, the fragments comprise at least 11 amino acids. In some embodiments, the fragments comprise at least 12 amino acids. In some embodiments, the fragments comprise at least 13 amino acids. In some embodiments, the fragments comprise at least 14 amino acids. In some embodiments, the fragments comprise at least 15 amino acids. In some embodiments, the fragments comprise at least 16 amino acids. In some embodiments, the fragments comprise at least 17 amino acids. In some embodiments, the fragments comprise at least 18 amino acids. In some embodiments, the fragments comprise at least 19 amino acids. In some embodiments, the fragments comprise at least 20 amino acids. In some embodiments, the fragments comprise at least 21 amino acids. In some embodiments, the fragments comprise at least 22 amino acids. In some embodiments, the fragments comprise at least 23 amino acids. In some embodiments, the fragments comprise at least 24 amino acids. In some embodiments, the fragments comprise at least 25 amino acids. In some embodiments, the fragments comprise about 6-25 amino acids. In some embodiments, the fragments comprise about 7-25 amino acids. In some embodiments, the fragments comprise about 8-25 amino acids. In some embodiments, the fragments comprise about 8-24 amino acids. In some embodiments, the fragments comprise about 8-23 amino acids. In some embodiments, the fragments comprise about 8-22 amino acids. In some embodiments, the fragments comprise about 8-21 amino acids. In some embodiments, the fragments comprise about 8-20 amino acids. In some embodiments, the fragments comprise about 8-19 amino acids. In some embodiments, the fragments comprise about 8-18 amino acids. In some embodiments, the fragments comprise about 8-17 amino acids. In some embodiments, the fragments comprise about 8-16 amino acids. In some embodiments, the fragments comprise about 8-15 amino acids. In some embodiments, the fragments comprise about 8-14 amino acids. In some embodiments, the fragments comprise about 9-14 amino acids. In some embodiments, the fragments comprise about 9-13 amino acids. In some embodiments, the fragments comprise about 9-12 amino acids. In some embodiments, the fragments comprise about 9-11 amino acids. In some embodiments, the fragments comprise about 9-10 amino acids.

In some embodiments, the fragments comprise at least 18 nucleotides. In some embodiments, the fragments comprise at least 21 nucleotides. In some embodiments, the fragments comprise at least 24 nucleotides. In some embodiments, the fragments comprise at least 27 nucleotides. In some embodiments, the fragments comprise at least 30 nucleotides. In some embodiments, the fragments comprise at least 33 nucleotides. In some embodiments, the fragments comprise at least 36 nucleotides. In some embodiments, the fragments comprise at least 39 nucleotides. In some embodiments, the fragments comprise at least 42 nucleotides. In some embodiments, the fragments comprise at least 45 nucleotides. In some embodiments, the fragments comprise at least 48 nucleotides. In some embodiments, the fragments comprise at least 51 nucleotides. In some embodiments, the fragments comprise at least 54 nucleotides. In some embodiments, the fragments comprise at least 57 nucleotides. In some embodiments, the fragments comprise at least 60 nucleotides. In some embodiments, the fragments comprise at least 63 nucleotides. In some embodiments, the fragments comprise at least 66 nucleotides. In some embodiments, the fragments comprise at least 69 nucleotides. In some embodiments, the fragments comprise at least 72 nucleotides. In some embodiments, the fragments comprise at least 75 nucleotides. In some embodiments, the fragments comprise about 18-75 nucleotides. In some embodiments, the fragments comprise about 21-75 nucleotides. In some embodiments, the fragments comprise about 24-75 nucleotides. In some embodiments, the fragments comprise about 24-72 nucleotides. In some embodiments, the fragments comprise about 24-69 nucleotides. In some embodiments, the fragments comprise about 24-66 nucleotides. In some embodiments, the fragments comprise about 24-63 nucleotides. In some embodiments, the fragments comprise about 24-60 nucleotides. In some embodiments, the fragments comprise about 24-57 nucleotides. In some embodiments, the fragments comprise about 24-54 nucleotides. In some embodiments, the fragments comprise about 24-51 nucleotides. In some embodiments, the fragments comprise about 24-48 nucleotides. In some embodiments, the fragments comprise about 24-45 nucleotides. In some embodiments, the fragments comprise about 24-42 nucleotides. In some embodiments, the fragments comprise about 27-42 nucleotides. In some embodiments, the fragments comprise about 27-39 nucleotides. In some embodiments, the fragments comprise about 27-36 nucleotides. In some embodiments, the fragments comprise about 27-33 nucleotides. In some embodiments, the fragments comprise about 27-30 nucleotides.

In some embodiments, the fragments comprise one or more of SEQ ID NOs: 387, 388, 389, 390, 391, 392, 393, 394, 395, 396, 397, 398, 399, 400, 401, 402, 403, 404, 405, 406, 407, 408, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, or 621.

In some embodiments, the fragments comprise one or more of SEQ ID NOs: 377, 378, 415, 417, 418, 420, 502, 518, 526, 527, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 74, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 487, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, or 548.

The methods can comprise evaluating the presence of any combination of the above prostate cancer neoantigens, fragments of the prostate cancer neoantigens, polynucleotides encoding the prostate cancer neoantigens, and/or fragments of the polynucleotides encoding the prostate cancer neoantigens.

The sample from the subject can comprise any biological sample known to contain or suspected of containing tumor material. In some embodiments, the sample can comprise a prostate cancer tissue sample. In some embodiments, the sample can contain other types of materials containing cancer cells or biological derivatives from cancer cells (exosomes, apoptotic modies, circulating nucleic acids, etc.).

The disclosed methods can be used to diagnose a subject with any form of prostate cancer. “Prostate cancer” as used herein is meant to include all types of cancerous growths within prostate or oncogenic processes, metastatic tissues or malignantly transformed cells, tissues, or organs, irrespective of histopathology type or stage of invasiveness. The disclosed methods can be used to diagnose, for example, a localized prostate adenocarcinoma, a relapsed prostate cancer, a refractory prostate cancer, a metastatic prostate cancer, a castration resistant prostate cancer, or any combination thereof. In some embodiments, the prostate cancer is an adenocarcinoma. In some embodiments, the prostate cancer is a metastatic prostate cancer. In some embodiments, the prostate cancer has metastasized to rectum, lymph node or bone, or any combination thereof. In some embodiments, the prostate cancer is a relapsed or a refractory prostate cancer. In some embodiments, the prostate cancer is a castration resistant prostate cancer. In some embodiments, the prostate cancer is sensitive to an androgen deprivation therapy. In some embodiments, the prostate cancer is insensitive to the androgen deprivation therapy.

In some embodiments, the subject is treatment naïve. In some embodiments, the subject has received androgen deprivation therapy. In some embodiments, the subject has an elevated level of prostate specific antigen (PSA). PSA is elevated in a subject when the level is typically about >4.0 ng/mL. In some instances, elevated PSA may refer to level of >3.0 ng/mL. PSA levels may also be compared to post-androgen deprivation therapy levels.

### Methods of Treatment, Uses and Administration

Methods of treating prostate cancer in a subject are also provided. The methods can comprise administering a therapeutically effective amount of a prostate cancer vaccine to the subject to thereby treat the prostate cancer, wherein the prostate cancer vaccine comprises a polynucleotide encoding one or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof.

The methods of treating prostate cancer in a subject can comprise evaluating the presence of any one or more of the prostate cancer neoantigens listed in the methods of diagnosis section above and administering a therapeutically effective amount of a prostate cancer vaccine to the subject to thereby treat the prostate cancer. In some embodiments, the methods can comprise:
a) evaluating the presence of one or more prostate cancer neoantigens in a sample from the subject, the one or more prostate cancer neoantigens comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof; and
b) administering a therapeutically effective amount of a prostate cancer vaccine to the subject to thereby treat the prostate cancer.

The methods can comprise evaluating the presence of one or more prostate cancer neoantigens comprising the amino acid sequence of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, fragments of the preceding sequences, or any combination thereof.

The methods can comprise evaluating the presence of one or more prostate cancer neoantigens comprising the amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, 223, fragments of the preceding sequences, or any combination thereof.

The methods can comprise evaluating the presence of each of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, and 223.

The sample from the subject from which the neoantigens are evaluated can comprise any biological sample known to contain or suspected of containing tumor material including, for example, a prostate cancer tissue sample or other types of materials containing cancer cells or biological derivatives from cancer cells (exosomes, apoptotic modies, circulating nucleic acids, etc.). In some embodiments, the sample from the subject from which the neoantigens are evaluated can comprise a prostate cancer tissue sample.

The presence of the one or more prostate cancer neoantigens can be evaluated by, for example, PCR, qPCR, various forms of nucleic acid sequencing (including but not limited to Illumina, Ion Torrent, Pacific Bioscience, Oxford Nanopore platforms), and various hybridization based approaches (including not limited to Affymetrix Gene Chip or Nanostring platforms). In some embodiments, the presence of the one or more prostate cancer neoantigens is evaluated by qPCR. In some aspects, the methods further comprise, prior to performing the qPCR, extracting RNA from the sample from the subject and synthesizing cDNA from the extracted RNA. The RNA extracted from the subject can correspond to a polynucleotide sequence comprising SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 380, 382, 384, 386, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 503, 504, 505, 506, 507, 508, 509, 510, 511, 512, 513, 514, 515, 516, 517, 519, 520, 521, 522, 523, 524, 525, 528, 529, 530, 531, 532, 533, 534, 535, 536, 537, 538, 539, 540, fragments of the preceding sequences, or any combination thereof.

In some embodiments, the methods of treating prostate cancer in a subject can further comprise, prior to administering the therapeutically effective amount of a prostate cancer vaccine, evaluating the expression of one or more prostate cancer biomarkers in a sample from the subject. The one or more prostate cancer biomarkers in a sample from the subject can comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, ROR1, FGF8, NKX2-2, EDIL3, RELN, FGF9, AKR1C4, CLUL1, KISSIR, CYP3A5, CYP17A1, SFRP4, HNF1A, CALCR, SYP, MSLN, or any combination thereof. Accordingly, the methods of treating prostate cancer in a subject can comprise:
a) evaluating the presence of one or more prostate cancer neoantigens in a sample from the subject, the one or more prostate cancer neoantigens comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof; and;
b) evaluating expression of one or more prostate cancer biomarkers in a sample from the subject, wherein the one or more prostate cancer biomarkers comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, ROR1, FGF8, NKX2-2, EDIL3, RELN, FGF9, AKR1C4, CLUL1, KISSIR, CYP3A5, CYP17A1, SFRP4, HNF1A, CALCR, SYP, MSLN, or any combination thereof; and
c) administering a therapeutically effective amount of a prostate cancer vaccine to the subject.

The sample from the subject from which the prostate cancer biomarkers is evaluated can comprise any biological sample known to contain or suspected of containing tumor material including, for example, a prostate cancer tissue sample or other types of materials containing cancer cells or biological derivatives from cancer cells (exosomes, apoptotic modies, circulating nucleic acids, etc.). In some embodiments, the sample is a plasma sample. In some aspects, the sample is from plasma exosomes. In some embodiments, the sample is a blood sample.

The one or more prostate cancer biomarkers can comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, or combinations thereof. In some embodiments, the one or more prostate cancer biomarkers are from a plasma sample. In some aspects, the one or more prostate cancer biomarkers are from plasma exosomes.

The one or more prostate cancer biomarkers can comprise: HPN, ROR1, FLNC, GPR39, FGF8, NKX2-2, MUC1, NKX3-1, EDIL3, LGR5, FGFR4, STEAP1, ATF3, RELN, UGT2B17, KLK3, C9orf152, GNMT, METTL7A, FGF9, SPDEF, FOXA1, AKR1C4, GREB1, CLUL1, TMEFF2, HOXB13, KLK2, NPY, GRHL2, STEAP2, THBS2, KISSIR, KRT8, TNFRSF19, CYP3A5, KLK4, IDO1, FOLH1, NROB1, EPHA3, CYP17A1, SFRP4, KRT18, TSPAN1, HNF1A, ADAMTS15, ACPP, CALCR, SYP, AZGP1, AR, ARv3, MSLN, TMPRSS2:ERG, and combinations thereof. In some embodiments, the one or more prostate cancer biomarkers are from a blood sample.

The presence of the one or more prostate cancer biomarkers can be evaluated by, for example, PCR, qPCR, various forms of nucleic acid sequencing (including but not limited to Illumina, Ion Torrent, Pacific Bioscience, Oxford Nanopore platforms), and various hybridization based approaches (including not limited to Affymetrix Gene Chip or Nanostring platforms). In some embodiments, the presence of the one or more prostate cancer biomarkers is evaluated by qPCR. In some aspects, the methods further comprise, prior to performing the qPCR, extracting RNA from the sample from the subject and synthesizing cDNA from the extracted RNA.

In some embodiments, the methods can further comprise, after administering the therapeutically effective amount of the prostate cancer vaccine, evaluating the expression of the one or more prostate cancer biomarkers evaluated in step b), wherein a decrease in expression compared to the expression in step b) is indicative of responsiveness to the prostate cancer vaccine. In such embodiments, the expression of the one or more prostate cancer biomarkers detected in step b) is the baseline expression of the cancer biomarker. Evaluating the expression of the one or more prostate cancer biomarkers after administering the therapeutically effective amount of the prostate cancer vaccine can provide an indication of responsiveness/therapeutic efficacy. In some embodiments, the collective expression of the biomarkers can determine whether the patient has responded to treatment. In some embodiments, a decrease in expression of the one or more prostate cancer biomarkers after administering the prostate cancer vaccine compared to the expression prior to administering the prostate cancer vaccine is indicative of responsiveness to the prostate cancer vaccine.

The prostate cancer vaccine can comprise one or more polynucleotides, one or more polypeptides, and/or one or more recombinant viruses. The prostate cancer vaccine can comprise one or more polynucleotides selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 380, 382, 384, 386, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 503, 504, 505, 506, 507, 508, 509, 510, 511, 512, 513, 514, 515, 516, 517, 519, 520, 521, 522, 523, 524, 525, 528, 529, 530, 531, 532, 533, 534, 535, 536, 537, 538, 539, 540, and fragments of the preceding sequences.

In some embodiments, the prostate cancer vaccine comprises:
a) one or more polynucleotides selected from the group consisting of SEQ ID NOs: 276, 382, 334, 338, 270, 254, 310, 326, 272, 306, 252, 246, 262, 266, 318, 256, 278, 298, 286, 448, 450, 453, 455, 380, 344, 212, 350, 214, 216, 222, 220, 226, 346, 354, 236, 224, 168, 172, 20, 24, 178, and fragments thereof;
b) one or more polynucleotides selected from the group consisting of SEQ ID NOs: 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and fragments thereof; or
c) one or more polynucleotides selected from the group consisting of SEQ ID NOs: 500, 501, 461, 503, 504, 505, 506, 507, 508, 509, 510, 511, 512, 513, 514, 515, 516, 517, 477, 519, 520, 521, 522, 523, 524, 525, 485, 486, 528, 529, 530, 531, 532, 533, 534, 535, 536, 537, 538, 539, 540, and fragments thereof.

In some embodiments, the prostate cancer vaccine comprises a polynucleotide sequence of SEQ ID NOs: 542, 551, 544, or 553.

The prostate cancer vaccine can comprise a polynucleotide encoding one or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, and fragments of the preceding sequences.

Through the validation process, 41 neoantigens (SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, and 177) were identified as particularly useful to be included into a prostate cancer vaccine based on their expression profile, prevalence, and in vitro immunogenicity. Thus, in some embodiments, the prostate cancer vaccine can comprise a polynucleotide encoding one or more polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, and fragments thereof. In some embodiments, the prostate cancer vaccine can comprise a polynucleotide encoding a polypeptide of any one of SEQ ID NOs: 541, 550, 554, 555, 556, 623, 624, 543, 552, 557, 558, 559, 625, or 626. It is expected that any combination of the 41 neoantigens can be utilized to generate a prostate cancer vaccine that can be delivered to a subject via any available delivery vehicles and any form available, such as peptides, DNA, RNA, replicons, or using viral delivery. The 41 neoantigens may be assembled into polynucleotides encoding polypeptides in any neoantigen order, and the neoantigen order may differ between the various delivery options. In general, assembly of the neoantigens into a particular order may be based on generating a minimum number of junctional epitopes utilizing known algorithms. Exemplary orders of the neoantigens are provided as SEQ ID NOs: 541, 550, 554, 555, 556, 623, 624, 543, 552, 557, 558, 559, 625 or 626 as described herein and throughout the examples.

The polynucleotide can be DNA or RNA. Suitable RNA molecules include mRNA or self-replicating RNA. In some embodiments, the polynucleotide comprises a promoter, an enhancer, a polyadenylation site, a Kozak sequence, a stop codon, a T cell enhancer (TCE), or any combination thereof. In some embodiments, the promoter comprises a CMV promoter or a vaccinia P7.5 promoter. In some embodiments, the TCE is encoded by a polynucleotide of SEQ ID NO: 546, the CMV promoter comprises a polynucleotide of SEQ ID NO: 628, the vaccinia P7.5 promoter comprises a polynucleotide of SEQ ID NO: 630, and the polyadenylation site comprises a bovine growth hormone polyadenylation site of SEQ ID NO: 629.

The prostate cancer vaccine can comprise one or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, and fragments of the preceding sequences. In some embodiments, the prostate cancer vaccine can comprise one or more polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, and fragments thereof. In some embodiments, the prostate cancer vaccine can comprise a polypeptide comprising the amino acid sequence of any one of SEQ ID NOs: 541, 550, 554, 555, 556, 623, 624, 543, 552, 557, 558, 559, 625, or 626.

The polypeptides and polynucleotides may be attached to nanoparticles for delivery to a subject. Delivery of the polypeptides and polynucleotides using nanoparticles may eliminate the need to include a virus or an adjuvant in the vaccine composition. The polynucleotide may be DNA or RNA. The nanoparticles may contain immune danger signals that help to effectively induce an immune response to the peptides. The nanoparticles may induce dendritic cell (DC) activation and maturation, required for a robust immune response. The nanoparticles may contain non-self components that improve uptake of the nanoparticles and thus the peptides by cells, such as antigen presenting cells.

The nanoparticles are typically from about 1 nm to about 100 nm in diameter, such as about 20 nm to about 40 nm. Nanoparticles with a mean diameter of 20 to 40 nm may facilitate uptake of the nanoparticle to the cytosol (see. e.g. WO2019/135086). Exemplary nanoparticles are polymeric nanoparticles, inorganic nanoparticles, liposomes, lipid nanoparticles (LNP), an immune stimulating complex (ISCOM), a virus-like particle (VLP), or a self-assembling protein. The nanoparticles may be calcium phosphate nanoparticles, silicon nanoparticles or gold nanoparticles. The polymeric nanoparticles may comprise one or more synthetic polymers, such as poly(d,l-lactide-co-glycolide) (PLG), poly(d,l-lactic-coglycolic acid) (PLGA), poly(g-glutamic acid) (g-PGA)m poly(ethylene glycol) (PEG), or polystyrene or one or more natural polymers such as a polysaccharide, for example pullulan, alginate, inulin, and chitosan. The use of a polymeric nanoparticles may be advantageous due to the properties of the polymers that may be include in the nanoparticle. For instance, the natural and synthetic polymers recited above may have good biocompatibility and biodegradability, a non-toxic nature, and/or the ability to be manipulated into desired shapes and sizes. The polymeric nanoparticle may also form hydrogel nanoparticles, hydrophilic three-dimensional polymer networks with favorable properties including flexible mesh size, large surface area for multivalent conjugation, high water content, and high loading capacity for antigens. Polymers such as Poly(L-lactic acid) (PLA), PLGA, PEG, and polysaccharides are suitable for forming hydrogel nanoparticles. Inorganic nanoparticles typically have a rigid structure and comprise a shell in which an antigen is encapsulated or a core to which the antigen may be covalently attached. The core may comprise one or more atoms such as gold (Au), silver (Ag), copper (Cu) atoms, Au/Ag, Au/Cu, Au/Ag/Cu, Au/Pt, Au/Pd or Au/Ag/Cu/Pd or calcium phosphate (CaP).

The nanoparticles may be liposomes. Liposomes are typically formed from biodegradable, non-toxic phospholipids and comprise a self-assembling phospholipid bilayer shell with an aqueous core. Liposomes may be an unilamellar vesicle comprising a single phospholipid bilayer, or a multilamellar vesicle that comprises several concentric phospholipid shells separated by layers of water. As a consequence, liposomes may be tailored to incorporate either hydrophilic molecules into the aqueous core or hydrophobic molecules within the phospholipid bilayers. Liposomes may encapsulate antigens such as the disclosed polypeptides or fragments thereof within the core for delivery. Liposomes and liposomal formulations can be prepared according to standard methods and are well known in the art, see, e.g., Remington's; Akimaru, 1995, Cytokines Mol. Ther. 1: 197-210; Alving, 1995, Immunol. Rev. 145: 5-31; Szoka, 1980, Ann. Rev. Biophys. Bioeng. 9: 467; U.S. Pat. Nos. 4,235,871; 4,501,728; and 4,837,028. The liposomes may comprise a targeting molecule for targeting liposome complexes to a particular cell type. Targeting molecule may comprise a binding partner (e.g., a ligand or receptor) for a biomolecule (e.g., a receptor or ligand) on the surface of a blood vessel or a cell found in a target tissue. Liposome charge is an important determinant in liposome clearance from the blood, with negatively charged liposomes being taken up more rapidly by the reticuloendothelial system (Juliano, 1975, Biochem. Biophys. Res. Commun. 63: 651) and thus having shorter half-lives in the bloodstream. Incorporating phosphatidylethanolamine derivatives enhances the circulation time by preventing liposomal aggregation. For example, incorporation of N-(omega-carboxy)acylamidophosphatidylethanolamines into large unilamellar vesicles of L-alpha-distearoylphosphatidylcholine dramatically increases the in vivo liposomal circulation lifetime (see, e.g., Ahl, 1997, Biochim. Biophys. Acta 1329: 370-382). Typically, liposomes are prepared with about 5 to 15 mole percent negatively charged phospholipids, such as phosphatidylglycerol, phosphatidylserine, or phosphatidyl-inositol. Added negatively charged phospholipids, such as phosphatidylglycerol, also serve to prevent spontaneous liposome aggregation, and thus minimize the risk of undersized liposomal aggregate formation. Membrane-rigidifying agents, such as sphingomyelin or a saturated neutral phospholipid, at a concentration of at least about 50 mole percent, and 5 to 15 mole percent of monosialylganglioside can also impart desirably liposome properties, such as rigidity (see, e.g., U.S. Pat. No. 4,837,028). Additionally, the liposome suspension can include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

The nanoparticles may be lipid nanoparticles (LNP). LNPs are similar to liposomes but have slightly different function and composition. LNPs are designed toward encapsulating polynucleotides, such as DNA, mRNA, siRNA, and sRNA. Traditional liposomes contain an aqueous core surrounded by one or more lipid bilayers. LNPs may assume a micelle-like structure, encapsulating drug molecules in a non-aqueous core. LNPs typically contain a cationic lipid, a non-cationic lipid, and a lipid that prevents aggregation of the particle (e.g., a PEG-lipid conjugate). LNPs are useful for systemic applications, as they exhibit extended circulation lifetimes following intravenous (i.e.) injection and accumulate at distal sites (e.g., sites physically separated from the administration site). The LNPs may have a mean diameter of about 50 nm to about 150 nm, such as about 60 nm to about 130 nm, or about 70 nm to about 110 nm, or about 70 nm to about 90 nm, and are substantially nontoxic. Preparation of polynucleotide loaded LNPs are disclosed in, e.g., U.S. Pat. Nos. 5,976,567; 5,981,501; 6,534,484; 6,586,410; 6,815,432; and PCT Publication No. WO 96/40964. Polynucleotide containing LNPs are described for example in WO2019/191780.

The polynucleotides, and polypeptides of the disclosure can include multilamellar vesicles of heterogeneous sizes. For example, vesicle-forming lipids can be dissolved in a suitable organic solvent or solvent system and dried under vacuum or an inert gas to form a thin lipid film. If desired, the film can be redissolved in a suitable solvent, such as tertiary butanol, and then lyophilized to form a more homogeneous lipid mixture which is in a more easily hydrated powder like form. This film is covered with an aqueous solution of the polypeptide complex and allowed to hydrate, typically over a 15 to 60 minute period with agitation. The size distribution of the resulting multilamellar vesicles can be shifted toward smaller sizes by hydrating the lipids under more vigorous agitation conditions or by adding solubilizing detergents such as deoxycholate. The hydration medium may comprise the nucleic acid at a concentration which is desired in the interior volume of the liposomes in the final liposome suspension. Suitable lipids that may be used to form multilamellar vesicles include DOTMA (Feigner, et al., 1987, Proc. Natl. Acad. Sci. USA 84: 7413-7417), DOGS or Transfectain™ (Behr, et al., 1989, Proc. Natl. Acad. Sci. USA 86: 6982-6986), DNERIE or DORIE (Feigner, et al., Methods 5: 67-75), DC-CHOL (Gao and Huang, 1991, BBRC 179: 280-285), DOTAP™ (McLachlan, et al., 1995, Gene Therapy 2: 674-622), Lipofectamine™, and glycerolipid compounds (see, e.g., EP901463 and WO98/37916).

The nanoparticle may be an immune-stimulating complex (ISCOM). ISCOMs are cage-like particles which are typically formed from colloidal saponin-containing micelles. ISCOMs may comprise cholesterol, phospholipid (such as phosphatidylethanolamine or phosphatidylcholine), and saponin (such as Quil A from the tree Quillaia saponaria).

The nanoparticle may be a virus-like particle (VLP). VLPs are self-assembling nanoparticles that lack infectious nucleic acid, which are formed by self-assembly of biocompatible capsid protein. VLPs are typically about 20 to about 150 nm, such as about 20 to about 40 nm, about 30 to about 140 nm, about 40 to about 130 nm, about 50 to about 120 nm, about 60 to about 110 nm, about 70 to about 100 nm, or about 80 to about 90 nm in diameter. VLPs advantageously harness the power of evolved viral structure, which is naturally optimized for interaction with the immune system. The naturally-optimized nanoparticle size and repetitive structural order means that VLPs induce potent immune responses, even in the absence of adjuvant.

The nanoparticles may contain replicons that encode the polypeptides of the disclosure. The replicons may be DNA or RNA. “Replicon” refers to a viral nucleic acid that is capable of directing the generation of copies of itself and includes RNA as well as DNA. For example, double-stranded DNA versions of arterivirus genomes can be used to generate a single-stranded RNA transcript that constitutes an arterivirus replicon. Generally, a viral replicon contains the complete genome of the virus. “Sub-genomic replicon” refers to a viral nucleic acid that contains something less than the full complement of genes and other features of the viral genome, yet is still capable of directing the generation of copies of itself. For example, the sub-genomic replicons of arterivirus may contain most of the genes for the non-structural proteins of the virus, but are missing most of the genes coding for the structural proteins. Sub-genomic replicons are capable of directing the expression of all of the viral genes necessary for the replication of the viral sub-genome (replication of the sub-genomic replicon), without the production of viral particles.

“RNA replicon,” “self-replication RNA,” or “self-replicating RNA” refers to RNA which contains all of the genetic information required for directing its own amplification or self-replication within a permissive cell. To direct its own replication, the RNA molecule: 1) encodes polymerase, replicase, or other proteins which may interact with viral or host cell-derived proteins, nucleic acids or ribonucleoproteins to catalyze the RNA amplification process; and 2) contain cis-acting RNA sequences required for replication and transcription of the replicon-encoded RNA. Self-replicating RNA is typically derived from the genomes of positive strand RNA viruses and can be used as a basis of introducing foreign sequences to host cells by replacing viral sequences encoding structural or non-structural genes or inserting the foreign sequences 5′ or 3′ of the sequences encoding the structural or non-structural genes. Foreign sequences may also be introduced into the subgenomic regions of alphaviruses. Self-replicating RNA may be packaged into recombinant virus particles, such as recombinant alphavirus particles or alternatively delivered to the host using lipid nanoparticles (LNP). Self-replicating RNA may be at least 1 kb or at least 2 kb or at least 3 kb or at least 4 kb or at least 5 kb or at least 6 kb or at least 7 kb or at least 8 kb or at least 10 kb or at least 12 kb or at least 15 kb or at least 17 kb or at least 19 kb or at least 20 kb in size, or can be 100 bp-8 kb or 500 bp-8 kb or 500 bp-7 kb or 1-7 kb or 1-8 kb or 2-15 kb or 2-20 kb or 5-15 kb or 5-20 kb or 7-15 kb or 7-18 kb or 7-20 kb in size. Self-replicating RNAs are described, for example, in WO2017/180770, WO2018/075235, and WO2019143949A2.

Other molecules suitable for complexing with the polynucleotides or the polypeptides of the disclosure include cationic molecules, such as, polyamidoamine (Haensler and Szoka, 1993, Bioconjugate Chem. 4: 372-379), dendritic polylysine (Int. Pat. Publ. No. WO1995/24221), polyethylene irinine or polypropylene h-nine (Int. Pat. Publ. No. WO1996/02655), polylysine (U.S. Pat. No. 5,595,897), chitosan (U.S. Pat. No. 5,744,166), DNA-gelatin coarcervates (see, e.g., U.S. Pat. Nos. 6,207,195; 6,025,337; 5,972,707) or DEAE dextran (Lopata, et al., 1984, Nucleic Acid Res. 12: 5707-5717), dendrimers (see, e.g., WO1996/19240), or polyethylenimine (PEI) (see, e.g., Sun et al., 2014, Mol Med Rep. 10(5):2657-2662).

In some embodiments, the prostate cancer vaccine comprises one or more recombinant viruses. Suitable recombinant viruses can be derived from an adenovirus (Ad), a poxvirus, an adeno-associated virus (AAV), or a retrovirus.

Adenoviruses may be derived from human adenovirus (Ad) but also from adenoviruses that infect other species, such as bovine adenovirus (e.g. bovine adenovirus 3, BAdV3), a canine adenovirus (e.g. CAdV2), a porcine adenovirus (e.g. PAdV3 or 5), or great apes, such as Chimpanzee (Pan), Gorilla (Gorilla), Orangutan (Pongo), Bonobo (Pan paniscus) and common chimpanzee (Pan troglodytes). Typically, naturally occurring great ape adenoviruses are isolated from stool samples of the respective great ape.

Human adenoviruses may be derived from various adenovirus serotypes, for example, from human adenovirus serotypes hAd5, hAd7, hAdl1, hAd26, hAd34, hAd35, hAd48, hAd49, or hAd50 (the serotypes are also referred to as Ad5, Ad7, Ad11, Ad26, Ad34, Ad35, Ad48, Ad49, or Ad50).

Great ape adenoviruses may be derived from various adenovirus serotypes, for example, from great ape adenovirus serotypes GAd20, GAd19, GAd21, GAd25, GAd26, GAd27, GAd28, GAd29, GAd30, GAd31, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAd1, PanAd2, or PanAd3.

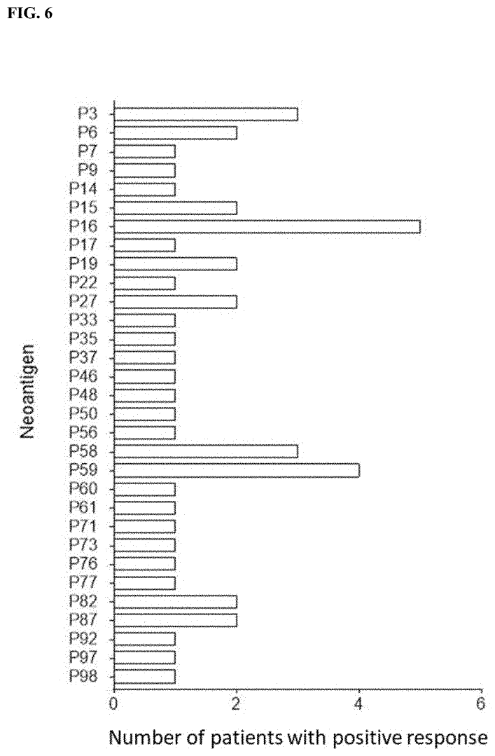

Adenoviruses are known in the art. The sequences of most of the human and nonhuman adenoviruses are known, and for others can be obtained using routine procedures. An exemplary genome sequence of Ad26 is found in GenBank Accession number EF153474 and in SEQ ID NO: 1 of Int. Pat. Publ. No. WO2007/104792. An exemplary genome sequence of Ad35 is found in FIG. 6 of Int. Pat. Publ. No. WO2000/70071. Ad26 is described, for example, in Int. Pat. Publ. No. WO2007/104792. Ad35 is described, for example, in U.S. Pat. No. 7,270,811 and Int. Pat. Publ. No. WO2000/70071. ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, and ChAd82 are described in WO2005/071093. PanAd1, PanAd2, PanAd3, ChAd55, ChAd73, ChAd83, ChAd146, and ChAd147 are described in Int. Pat. Publ. No. WO2010/086189.

Adenoviruses are engineered to comprise at least one functional deletion or a complete removal of a gene product that is essential for viral replication, such as one or more of the adenoviral regions E1, E2, and E4, therefore rendering the adenovirus to be incapable of replication. The deletion of the E1 region may comprise deletion of EIA, EIB 55K, or EIB 21K, or any combination thereof. Replication deficient adenoviruses are propagated by providing the proteins encoded by the deleted region(s) in trans by the producer cell by utilizing helper plasmids or engineering the produce cell to express the required proteins. Adenoviruses may also have a deletion in the E3 region, which is dispensable for replication, and hence such a deletion does not have to be complemented. The adenovirus may comprise a functional deletion or a complete removal of the E1 region and at least part of the E3 region. The adenovirus may further comprise a functional deletion or a complete removal of the E4 region and/or the E2 region. Suitable producer cells that can be utilized are human retina cells immortalized by E1, e.g. 911 or PER.C6 cells (see, e.g., U.S. Pat. No. 5,994,128), E1-transformed amniocytes (See, e.g., EP 1230354), E1-transformed A549 cells (see e.g. Int. Pat. Publ. No. WO1998/39411, U.S. Pat. No. 5,891,690). Ad26 comprising a functional E1 coding region that is sufficient for viral replication, a deletion in the E3 coding region, and a deletion in the E4 coding region may be used, provided that E4 open reading frame 6/7 is not deleted (see e.g. U.S. Pat. No. 9,750,801)

In some embodiments, the adenovirus is a human adenovirus (Ad). In some embodiments, the Ad is derived from Ad5. In some embodiments, the Ad is derived from Ad11. In some embodiments, the Ad is derived from Ad26. In some embodiments, the Ad is derived from Ad34. In some embodiments, the Ad is derived from Ad35. In some embodiments, the Ad is derived from Ad48. In some embodiments, the Ad is derived from Ad49. In some embodiments, the Ad is derived from Ad50.

In some embodiments, the adenovirus is a great ape adenovirus (GAd). In some embodiments, the GAd is derived from GAd20. In some embodiments, the GAd is derived from GAd19. In some embodiments, the GAd is derived from GAd21. In some embodiments, the GAd is derived from GAd25. In some embodiments, the GAd is derived from GAd26. In some embodiments, the GAd is derived from GAd27. In some embodiments, the GAd is derived from GAd28. In some embodiments, the GAd is derived from GAd29. In some embodiments, the GAd is derived from GAd30. In some embodiments, the GAd is derived from GAd31. In some embodiments, the GAd is derived from ChAd4. In some embodiments, the GAd is derived from ChAd5. In some embodiments, the GAd is derived from ChAd6. In some embodiments, the GAd is derived from ChAd7. In some embodiments, the GAd is derived from ChAd8. In some embodiments, the GAd is derived from ChAd9. In some embodiments, the GAd is derived from ChAd20. In some embodiments, the GAd is derived from ChAd22. In some embodiments, the GAd is derived from ChAd24. In some embodiments, the GAd is derived from ChAd26. In some embodiments, the GAd is derived from ChAd30. In some embodiments, the GAd is derived from ChAd31. In some embodiments, the GAd is derived from ChAd32. In some embodiments, the GAd is derived from ChAd33. In some embodiments, the GAd is derived from ChAd37. In some embodiments, the GAd is derived from ChAd38. In some embodiments, the GAd is derived from ChAd44. In some embodiments, the GAd is derived from ChAd55. In some embodiments, the GAd is derived from ChAd63. In some embodiments, the GAd is derived from ChAd68. In some embodiments, the GAd is derived from ChAd73. In some embodiments, the GAd is derived from ChAd82. In some embodiments, the GAd is derived from ChAd83.

GAd19-21 and GAd25-31 are described in Int. Pat. Publ. No. WO2019/008111 and represent strains with high immunogenicity and no pre-existing immunity in the general human population. The polynucleotide sequence of GAd20 genome is shown in SEQ ID NO: 622 as disclosed in WO2019/008111.

The disclosed polynucleotides may be inserted into a site or region (insertion region) in the virus that does not affect virus viability of the resultant recombinant virus. The polynucleotides may be inserted into the deleted E1 region in parallel (transcribed 5′ to 3′) or anti-parallel (transcribed in a 3′ to 5′ direction relative to the vector backbone) orientation. In addition, appropriate transcriptional regulatory elements that are capable of directing expression of the polypeptides in the mammalian host cells that the virus is being prepared for use may be operatively linked to the polynucleotides. “Operatively linked” sequences include both expression control sequences that are contiguous with the nucleic acid sequences that they regulate and regulatory sequences that act in trans, or at a distance to control the regulated nucleic acid sequence.

Recombinant adenoviral particles may be prepared and propagated according to any conventional technique in the field of the art (e.g., Int. Pat. Publ. No. WO1996/17070) using a complementation cell line or a helper virus, which supplies in trans the missing viral genes necessary for viral replication. The cell lines 293 (Graham et al., 1977, J. Gen. Virol. 36: 59-72), PER.C6 (see e.g. U.S. Pat. No. 5,994,128), E1 A549 and 911 are commonly used to complement E1 deletions. Other cell lines have been engineered to complement defective vectors (Yeh, et al., 1996, J. Virol. 70: 559-565; Kroughak and Graham, 1995, Human Gene Ther. 6: 1575-1586; Wang, et al., 1995, Gene Ther. 2: 775-783; Lusky, et al., 1998, J. Virol. 72: 2022-203; EP 919627 and Int. Pat. Publ. No. WO1997/04119). The adenoviral particles may be recovered from the culture supernatant but also from the cells after lysis and optionally further purified according to standard techniques (e.g., chromatography, ultracentrifugation, as described in Int. Pat. Publ. No. WO1996/27677, Int. Pat. Publ. No. WO1998/00524, Int. Pat. Publ. No. WO1998/26048 and Int. Pat. Publ. No. WO2000/50573). The construction and methods for propagating adenoviruses are also described in for example, U.S. Pat. Nos. 5,559,099, 5,837,511, 5,846,782, 5,851,806, 5,994,106, 5,994,128, 5,965,541, 5,981,225, 6,040,174, 6,020,191, and 6,113,913.

Poxvirus (Poxviridae) may be derived from smallpox virus (variola), vaccinia virus, cowpox virus, or monkeypox virus. Exemplary vaccinia viruses are the Copenhagen vaccinia virus (W), New York Attenuated Vaccinia Virus (NYVAC), ALVAC, TROVAC, and Modified Vaccinia Ankara (MVA).

MVA originates from the dermal vaccinia strain Ankara (Chorioallantois vaccinia Ankara (CVA) virus) that was maintained in the Vaccination Institute, Ankara, Turkey for many years and used as the basis for vaccination of humans. However, due to the often severe post-vaccinal complications associated with vaccinia viruses (VACV), there were several attempts to generate a more attenuated, safer smallpox vaccine. MVA has been generated by 516 serial passages on chicken embryo fibroblasts of the CVA virus (see Meyer et al., J. Gen. Virol., 72: 1031-1038 (1991) and U.S. Pat. No. 10,035,832). As a consequence of these long-term passages the resulting MVA virus deleted about 31 kilobases of its genomic sequence and, therefore, was described as highly host cell restricted to avian cells (Meyer, H. et al., Mapping of deletions in the genome of the highly attenuated vaccinia virus MVA and their influence on virulence, J. Gen. Virol. 72, 1031-1038, 1991; Meisinger-Henschel et al., Genomic sequence of chorioallantois vaccinia virus Ankara, the ancestor of modified vaccinia virus Ankara, J. Gen. Virol. 88, 3249-3259, 2007). Comparison of the MVA genome to its parent, CVA, revealed 6 major deletions of genomic DNA (deletion I, II, III, IV, V, and VI), totaling 31,000 basepairs. (Meyer et al., J. Gen. Virol. 72:1031-8 (1991)). It was shown in a variety of animal models that the resulting MVA was significantly avirulent (Mayr, A. & Danner, K. Vaccination against pox diseases under immunosuppressive conditions, Dev. Biol. Stand. 41: 225-34, 1978). Being that many passages were used to attenuate MVA, there are a number of different strains or isolates, depending on the passage number in CEF cells, such as MVA 476 MG/14/78, MVA-571, MVA-572, MVA-574, MVA-575, and MVA-BN. MVA 476 MG/14/78 is described, for example, in Int. Pat. Publ. No. WO2019/115816A1. MVA-572 strain was deposited at the European Collection of Animal Cell Cultures (“ECACC”), Health Protection Agency, Microbiology Services, Porton Down, Salisbury SP4 0JG, United Kingdom (“UK”), under the deposit number ECACC 94012707 on Jan. 27, 1994. MVA-575 strain was deposited at the ECACC under deposit number ECACC 00120707 on Dec. 7, 2000; MVA-Bavarian Nordic (“MVA-BN”) strain was deposited at the ECACC under deposit number V00080038 on Aug. 30, 2000. The genome sequences of MVA-BN and MVA-572 are available at GenBank (Accession numbers DQ983238 and DQ983237, respectively). The genome sequences of other MVA strains can be obtained using standard sequencing methods.

The disclosed viruses may be derived from any MVA strain or further derivatives of the MVA strain. A further exemplary MVA strain is deposit VR-1508, deposited at the American Type Culture collection (ATCC), Manassas, Va. 20108, USA.

“Derivatives” of MVA refer to viruses exhibiting essentially the same characteristics as the parent MVA, but exhibiting differences in one or more parts of their genomes. In some embodiments, the MVA is derived from MVA 476 MG/14/78. In some embodiments, the MVA is derived from MVA-571. In some embodiments, the MVA is derived from MVA-572. In some embodiments, the MVA is derived from MVA-574. In some embodiments, the MVA is derived from MVA-575. In some embodiments, the MVA is derived from MVA-BN.

The disclosed polynucleotides may be inserted into a site or region (insertion region) in the MVA virus that does not affect viability of the resultant recombinant virus. Such regions can be readily identified by testing segments of virus DNA for regions that allow recombinant formation without seriously affecting viability of the recombinant virus. The thymidine kinase (TK) gene is an insertion region that may be used and is present in many viruses, such as in all examined poxvirus genomes. Additionally, MVA contains 6 natural deletion sites, each of which may be used as insertion sites (e.g. deletion I, II, III, IV, V, and VI; see e.g. U.S. Pat. Nos. 5,185,146 and 6,440,442). One or more intergenic regions (IGR) of the MVA may also be used as an insertion site, such as IGRs IGR07/08, IGR 44/45, IGR 64/65, IGR 88/89, IGR 136/137, and IGR 148/149 (see e.g. U.S. Pat. Publ. No. 2018/0064803). Additional suitable insertion sites are described in Int. Pat. Publ. No. WO2005/048957.

Recombinant poxviral particles such as MVA can be prepared as described in the art (Piccini, et al., 1987, Methods of Enzymology 153: 545-563; U.S. Pat. Nos. 4,769,330; 4,772,848; 4,603,112; 5,100,587; and 5,179,993). In an exemplary method, the DNA sequence to be inserted into the virus can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences. MVA particles may be recovered from the culture supernatant or from the cultured cells after a lysis step (e.g., chemical lysis, freezing/thawing, osmotic shock, sonication and the like). Consecutive rounds of plaque purification can be used to remove contaminating wild type virus. Viral particles can then be purified using the techniques known in the art (e.g., chromatographic methods or ultracentrifugation on cesium chloride or sucrose gradients).

Other viruses include those derived from human adeno-associated viruses, such as AAV-2 (adeno-associated virus type 2). An attractive feature of AAV is that they do not express any viral genes. The only viral DNA sequences included in the AAV are the 145 bp inverted terminal repeats (ITR). Thus, as in immunization with naked DNA, the only gene expressed is that of the antigen, or antigen chimera. Additionally, AAVs are known to transduce both dividing and non-dividing cells, such as human peripheral blood monocyte-derived dendritic cells, with persistent transgene expression, and with the possibility of oral and intranasal delivery for generation of mucosal immunity. Moreover, the amount of DNA required appears to be much less by several orders of magnitude, with maximum responses at doses of 10¹⁰ to 10¹¹ particles or copies of DNA in contrast to naked DNA doses of 50 μg or about 10¹⁵ copies. AAVs are packaged by co-transfection of a suitable cell line (e.g., human 293 cells) with the DNA contained in the AAV ITR chimeric protein encoding constructs and an AAV helper plasmid ACG2 containing the AAV coding region (AAV rep and cap genes) without the ITRs. The cells are subsequently infected with the adenovirus. Viruses can be purified from cell lysates using methods known in the art (e.g., such as cesium chloride density gradient ultracentrifugation) and are validated to ensure that they are free of detectable replication-competent AAV or adenovirus (e.g., by a cytopathic effect bioassay).

Retroviruses may also be used. Retroviruses are a class of integrative viruses which replicate using a virus-encoded reverse transcriptase, to replicate the viral RNA genome into double stranded DNA which is integrated into chromosomal DNA of the infected cells (e.g., target cells). Such viruses include those derived from murine leukemia viruses, especially Moloney (Gilboa, et al., 1988, Adv. Exp. Med. Biol. 241: 29) or Friend's FB29 strains (Int. Pat. Publ. No. WO1995/01447). Generally, a retrovirus is deleted of all or part of the viral genes gag, pol, and env and retains 5′ and 3′ LTRs and an encapsidation sequence. These elements may be modified to increase expression level or stability of the retrovirus. Such modifications include the replacement of the retroviral encapsidation sequence by one of a retrotransposon such as VL30 (see, e.g., U.S. Pat. No. 5,747,323). The disclosed polynucleotides may be inserted downstream of the encapsidation sequence, such as in opposite direction relative to the retroviral genome. Retroviral particles are prepared in the presence of a helper virus or in an appropriate complementation (packaging) cell line which contains integrated into its genome the retroviral genes for which the retrovirus is defective (e.g. gag/pol and env). Such cell lines are described in the prior art (Miller and Rosman, 1989, BioTechniques 7: 980; Danos and Mulligan, 1988, Proc. Natl. Acad. Sci. USA 85: 6460; Markowitz, et al., 1988, Virol. 167: 400). The product of the env gene is responsible for the binding of the viral particle to the viral receptors present on the surface of the target cell and, therefore determines the host range of the retroviral particle. Packaging cell line, such as the PA317 cells (ATCC CRL 9078) or 293EI6 (WO97/35996) containing an amphotropic envelope protein may therefore be used to allow infection of human and other species' target cells. The retroviral particles are recovered from the culture supernatant and may optionally be further purified according to standard techniques (e.g. chromatography, ultracentrifugation).

The prostate cancer vaccine can comprise one or more recombinant viruses derived from, for example, hAd5, hAd7, hAdl1, hAd26, hAd34, hAd35, hAd48, hAd49, hAd50, GAd20, GAd19, GAd21, GAd25, GAd26, GAd27, GAd28, GAd29, GAd30, GAd31, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAd1, PanAd2, PanAd3, Copenhagen vaccinia virus (W), New York Attenuated Vaccinia Virus (NYVAC), ALVAC, TROVAC, modified vaccinia ankara (MVA), and combinations thereof.

The prostate cancer vaccine can comprise one or more recombinant viruses derived from GAd20, wherein the recombinant virus comprises a polynucleotide encoding one or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, and fragments of the preceding sequences. In some embodiments, the recombinant virus comprises a polynucleotide encoding one or more polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, and fragments thereof.

The prostate cancer vaccine can comprise one or more recombinant viruses derived from GAd20, wherein the recombinant virus comprises one or more polynucleotides selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 380, 382, 384, 386, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 503, 504, 505, 506, 507, 508, 509, 510, 511, 512, 513, 514, 515, 516, 517, 519, 520, 521, 522, 523, 524, 525, 528, 529, 530, 531, 532, 533, 534, 535, 536, 537, 538, 539, 540, and fragments of the preceding sequences.

In some embodiments, the prostate cancer vaccine comprise one or more recombinant viruses derived from GAd20, wherein the recombinant virus comprises:
a) one or more polynucleotides selected from the group consisting of SEQ ID NOs: 276, 382, 334, 338, 270, 254, 310, 326, 272, 306, 252, 246, 262, 266, 318, 256, 278, 298, 286, 448, 450, 453, 455, 380, 344, 212, 350, 214, 216, 222, 220, 226, 346, 354, 236, 224, 168, 172, 20, 24, 178, and fragments thereof; or
b) one or more polynucleotides selected from the group consisting of SEQ ID NOs: 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and fragments thereof.

In some aspects, the vaccine comprises a recombinant virus derived from GAd20 comprising a polynucleotide encoding a polypeptide of SEQ ID NO: 541, 550, 554, 555, 556, 623, or 624.

In some embodiments, the prostate cancer vaccine can comprise one or more recombinant viruses derived from MVA, wherein the recombinant virus comprises a polynucleotide encoding one or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, and fragments of the preceding sequences. In some embodiments, the recombinant virus can comprise a polynucleotide encoding one or more polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, and fragments thereof.

In some embodiments, the prostate cancer vaccine can comprise one or more recombinant viruses derived from MVA, wherein the recombinant virus comprises one or more polynucleotides selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 380, 382, 384, 386, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 503, 504, 505, 506, 507, 508, 509, 510, 511, 512, 513, 514, 515, 516, 517, 519, 520, 521, 522, 523, 524, 525, 528, 529, 530, 531, 532, 533, 534, 535, 536, 537, 538, 539, 540, and fragments of the preceding sequences.

In some embodiments, the prostate cancer vaccine can comprise one or more recombinant viruses derived from MVA, wherein the recombinant virus comprises:
a) one or more polynucleotides selected from the group consisting of SEQ ID NOs: 276, 382, 334, 338, 270, 254, 310, 326, 272, 306, 252, 246, 262, 266, 318, 256, 278, 298, 286, 448, 450, 453, 455, 380, 344, 212, 350, 214, 216, 222, 220, 226, 346, 354, 236, 224, 168, 172, 20, 24, 178, and fragments thereof; or
b) one or more polynucleotides selected from the group consisting of SEQ ID NOs: 500, 501, 461, 503, 504, 505, 506, 507, 508, 509, 510, 511, 512, 513, 514, 515, 516, 517, 477, 519, 520, 521, 522, 523, 524, 525, 485, 486, 528, 529, 530, 531, 532, 533, 534, 535, 536, 537, 538, 539, 540, and fragments thereof.

In some aspects, the vaccine comprises a recombinant virus derived from MVA comprising a polynucleotide encoding a polypeptide of SEQ ID NO: 543, 552, 557, 558, 559, 625, or 626.

In some embodiments, the prostate cancer vaccine can comprise one or more recombinant viruses derived from hAd26, wherein the recombinant virus comprises a polynucleotide encoding one or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, and fragments of the preceding sequences. In some embodiments, the recombinant virus can comprise a polynucleotide encoding one or more polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, and fragments thereof.

In some embodiments, the prostate cancer vaccine can comprise one or more recombinant viruses derived from hAd26, wherein the recombinant virus comprises one or more polynucleotides selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 380, 382, 384, 386, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 503, 504, 505, 506, 507, 508, 509, 510, 511, 512, 513, 514, 515, 516, 517, 519, 520, 521, 522, 523, 524, 525, 528, 529, 530, 531, 532, 533, 534, 535, 536, 537, 538, 539, 540, and fragments of the preceding sequences.

In some embodiments, the prostate cancer vaccine can comprise one or more recombinant viruses derived from hAd26, wherein the recombinant virus comprises:
a) one or more polynucleotides selected from the group consisting of SEQ ID NOs: 276, 382, 334, 338, 270, 254, 310, 326, 272, 306, 252, 246, 262, 266, 318, 256, 278, 298, 286, 448, 450, 453, 455, 380, 344, 212, 350, 214, 216, 222, 220, 226, 346, 354, 236, 224, 168, 172, 20, 24, 178, and fragments thereof;
b) one or more polynucleotides selected from the group consisting of SEQ ID NOs: 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and fragments thereof; or
c) one or more polynucleotides selected from the group consisting of SEQ ID NOs: 500, 501, 461, 503, 504, 505, 506, 507, 508, 509, 510, 511, 512, 513, 514, 515, 516, 517, 477, 519, 520, 521, 522, 523, 524, 525, 485, 486, 528, 529, 530, 531, 532, 533, 534, 535, 536, 537, 538, 539, 540, and fragments thereof.

In some aspects, the vaccine comprises a recombinant virus derived from hAd26 comprising a polynucleotide encoding a polypeptide of SEQ ID NO: 541, 550, 554, 555, 556, 623, or 624. In some aspects, the vaccine comprises a recombinant virus derived from hAd26 comprising a polynucleotide encoding a polypeptide of SEQ ID NO: 543, 552, 557, 558, 559, 625, or 626.

The vaccine can comprise a polynucleotide encoding a polypeptide of SEQ ID NO: 541, wherein the vaccine is a recombinant virus derived from GAd20. The vaccine can comprise a polynucleotide encoding a polypeptide of SEQ ID NO: 550, wherein the vaccine is a recombinant virus derived from GAd20. The vaccine can comprise a polynucleotide encoding a polypeptide of SEQ ID NO: 554, wherein the vaccine is a recombinant virus derived from GAd20. The vaccine can comprise a polynucleotide encoding a polypeptide of SEQ ID NO: 555, wherein the vaccine is a recombinant virus derived from GAd20. The vaccine can comprise a polynucleotide encoding a polypeptide of SEQ ID NO: 556, wherein the vaccine is a recombinant virus derived from GAd20. The vaccine can comprise a polynucleotide encoding a polypeptide of SEQ ID NO: 623, wherein the vaccine is a recombinant virus derived from GAd20. The vaccine can comprise a polynucleotide encoding a polypeptide of SEQ ID NO: 624, wherein the vaccine is a recombinant virus derived from GAd20. The vaccine can comprise a polynucleotide sequence of SEQ ID NO: 713, wherein the vaccine is a recombinant virus derived from GAd20. The vaccine can comprise a polynucleotide of SEQ ID NOs: 542, wherein the vaccine is a recombinant virus derived from GAd20. The vaccine can comprise a polynucleotide of SEQ ID NOs: 551, wherein the vaccine is a recombinant virus derived from GAd20.

The vaccine can comprise a polynucleotide encoding a polypeptide of SEQ ID NO: 543, wherein the vaccine is a recombinant virus derived from MVA. The vaccine can comprise a polynucleotide encoding a polypeptide of SEQ ID NO: 552, wherein the vaccine is a recombinant virus derived from MVA. The vaccine can comprise a polynucleotide encoding a polypeptide of SEQ ID NO: 557, wherein the vaccine is a recombinant virus derived from MVA. The vaccine can comprise a polynucleotide encoding a polypeptide of SEQ ID NO: 558, wherein the vaccine is a recombinant virus derived from MVA. The vaccine can comprise a polynucleotide encoding a polypeptide of SEQ ID NO: 559, wherein the vaccine is a recombinant virus derived from MVA. The vaccine can comprise a polynucleotide encoding a polypeptide of SEQ ID NO: 625, wherein the vaccine is a recombinant virus derived from MVA. The vaccine can comprise a polynucleotide encoding a polypeptide of SEQ ID NO: 626, wherein the vaccine is a recombinant virus derived from MVA. The vaccine can comprise a polynucleotide of SEQ ID NOs: 544, wherein the vaccine is a recombinant virus derived from MVA. The vaccine can comprise a polynucleotide of SEQ ID NOs: 553, wherein the vaccine is a recombinant virus derived from MVA.

In some embodiments, the vaccine comprising a recombinant virus derived from GAd20 is administered as a prime. In some embodiments, the vaccine comprising a recombinant virus derived from MVA is administered as a boost.

In some embodiments, the vaccine comprising the polynucleotide sequence encoding a polypeptide of SEQ ID NOs: 541 or 550 is administered as a prime. In some embodiments, the vaccine comprising the polynucleotide sequence encoding a polypeptide of SEQ ID NOs 543 or 552 is administered as a boost.

The methods of treatment can comprise administering to the subject a therapeutically effective amount of a first vaccine comprising any of the Ad26 for priming the immune response and administering to the subject a therapeutically effective amount of a second vaccine comprising any of the MVA for boosting the immune response, thereby treating the prostate cancer in the subject.

The methods of treatment can comprise administering to the subject a therapeutically effective amount of a first vaccine comprising any of the GAd for priming the immune response and administering to the subject a therapeutically effective amount of a second vaccine comprising any of the MVA for boosting the immune response, thereby treating the prostate cancer in the subject.

The methods of treatment can comprise administering to the subject a therapeutically effective amount of a first vaccine comprising any of the GAd20 for priming the immune response and administering to the subject a therapeutically effective amount of a second vaccine comprising any of the MVA for boosting the immune response, thereby treating the prostate cancer in the subject.

The methods of treatment can comprise:
a) administering to the subject a therapeutically effective amount of a vaccine comprising a polynucleotide encoding one, two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23 and 177, and fragments thereof as a prime; and
b) administering to the subject a therapeutically effective amount of a vaccine comprising a polynucleotide encoding one, two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23 and 177, and fragments thereof as a boost, thereby treating or preventing the prostate cancer in the subject.

The methods of treatment can comprise:
a) administering a first vaccine comprising a polynucleotide encoding a polypeptide comprising an amino acid sequence of SEQ ID NOs: 541, 550, 554, 555, 556, 623 or 624; and
b) a second vaccine comprising a polynucleotide encoding a polypeptide comprising the amino acid sequence of SEQ ID NOs: 543, 552, 557, 558, 559, 625 or 626.

The methods of treatment can comprise administering:
a) a first vaccine comprising a first polynucleotide encoding a first polypeptide, wherein the first polypeptide comprises an amino acid sequence of SEQ ID NO: 541; and
b) a second vaccine comprising a second polynucleotide encoding a second polypeptide, wherein the second polypeptide comprises the amino acid sequence of SEQ ID NO: 543.

The methods of treatment can comprise administering:
a) a first vaccine comprising a first polynucleotide encoding a first polypeptide, wherein the first polypeptide comprises an amino acid sequence of SEQ ID NO: 550; and
b) a second vaccine comprising a second polynucleotide encoding a second polypeptide, wherein the second polypeptide comprises the amino acid sequence of SEQ ID NO: 552.

The methods of treatment can comprise administering:
a) a first vaccine comprising a first polynucleotide encoding a first polypeptide, wherein the first polypeptide comprises an amino acid sequence of SEQ ID NO: 541, wherein the first vaccine is a recombinant GAd20; and
b) a second vaccine comprising a second polynucleotide encoding a second polypeptide, wherein the second polypeptide comprises the amino acid sequence of SEQ ID NO: 543, wherein the second vaccine is a recombinant MVA.

The methods of treatment can comprise administering:
a) a first vaccine comprising a first polynucleotide encoding a first polypeptide, wherein the first polypeptide comprises an amino acid sequence of SEQ ID NO: 550, wherein the first vaccine is a recombinant GAd20; and
b) a second vaccine comprising a second polynucleotide encoding a second polypeptide, wherein the second polypeptide comprises the amino acid sequence of SEQ ID NO: 552, wherein the second vaccine is a recombinant MVA.

In some embodiments, the first vaccine is administered between about 1-16 weeks prior to administering the second vaccine. In some embodiments, the first vaccine is administered about 1 week prior to administering the second vaccine. In some embodiments, the first vaccine is administered about 2 weeks prior to administering the second vaccine. In some embodiments, the first vaccine is administered about 3 weeks prior to administering the second vaccine. In some embodiments, the first vaccine is administered about 4 weeks prior to administering the second vaccine. In some embodiments, the first vaccine is administered about 5 weeks prior to administering the second vaccine. In some embodiments, the first vaccine is administered about 6 weeks prior to administering the second vaccine. In some embodiments, the first vaccine is administered about 7 weeks prior to administering the second vaccine. In some embodiments, the first vaccine is administered about 8 weeks prior to administering the second vaccine. In some embodiments, the first vaccine is administered about 9 weeks prior to administering the second vaccine. In some embodiments, the first vaccine is administered about 10 weeks prior to administering the second vaccine. In some embodiments, the first vaccine is administered about 11 weeks prior to administering the second vaccine. In some embodiments, the first vaccine is administered about 12 weeks prior to administering the second vaccine. In some embodiments, the first vaccine is administered about 13 weeks prior to administering the second vaccine. In some embodiments, the first vaccine is administered about 14 weeks prior to administering the second vaccine. In some embodiments, the first vaccine is administered about 15 weeks prior to administering the second vaccine. In some embodiments, the first vaccine is administered about 16 weeks prior to administering the second vaccine.

The polynucleotides, polypeptides, or recombinant vaccines can be administered, for example, intramuscularly, subcutaneously, intravenously, cutaneously, intradermally, or nasally. Intramuscular administration of the vaccines can be achieved by using a needle. Alternatively, a needleless injection device (using, e.g., Biojector™) or a freeze-dried powder containing the vaccine can be used.

For intravenous, cutaneous, or subcutaneous injection, or injection at the site of affliction, the vector may be the form of a parenterally acceptable aqueous solution which is pyrogen-free and has suitable pH, isotonicity and stability. Those of skill in the art are well able to prepare suitable solutions using, for example, isotonic vehicles such as Sodium Chloride Injection, Ringer's Injection, and Lactated Ringer's Injection. Preservatives, stabilizers, buffers, antioxidants and/or other additives can be included, as required. A slow-release formulation may also be employed.

Typically, administration will have a prophylactic aim to generate an immune response against the prostate neoantigens before development of symptoms of prostate cancer.

The vaccines are administered to a subject, giving rise to an immune response in the subject. An amount of the vaccine to induce a detectable immune response is considered an “immunologically effective dose.” The vaccines of the disclosure may induce a humoral as well as a cell-mediated immune response. In a typical embodiment the immune response is a protective immune response.

The actual amount administered, and rate and time-course of administration, will depend on the nature and severity of what is being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners.

In one exemplary regimen, the adenovirus is administered (e.g., intramuscularly) in a volume ranging between about 100 μL to about 10 ml containing concentrations of about 10⁴ to 10¹² virus particles/ml. The adenovirus may be administered in a volume ranging between 0.25 and 1.0 ml, such as in a volume of 0.5 ml. The adenovirus may be administered in an amount of about 10⁹ to about 10¹² viral particles (vp) to a human subject during one administration, more typically in an amount of about 10¹⁰ to about 10¹² vp.

In one exemplary regimen, the MVA is administered (e.g., intramuscularly) in a volume ranging between about 100 μl to about 10 ml of saline solution containing a dose of about 1×10⁷ TCID₅₀ to 1×10⁹ TCID₅₀ (50% Tissue Culture Infective Dose) or Inf.U. (Infectious Unit). The MVA may be administered in a volume ranging between 0.25 and 1.0 ml.

Boosting compositions may be administered two or more times, weeks or months after administration of the priming composition, for example, about 1 week, or 2 weeks, or 3 weeks, or 4 weeks, or 6 weeks, or 8 weeks, or 12 weeks, or 16 weeks, or 20 weeks, or 24 weeks, or 28 weeks, or 32 weeks, or one to two years after administration of the priming composition. Additional boosting compositions may be administered 6 weeks to 5 years after the boosting step (b), such as 6, 7, 8, 9, 10, 11, 12, 13, 14, 15, 16, 17, 18, 19, 20, 21, 22, 23, 24, or 25 weeks, or 7, 8, 9, 10, 11, or 12 months, or 2, 3, 4, or 5 years, after the initial boosting inoculation. Optionally, the further boosting step (c) can be repeated one or more times as needed.

The preparation of vaccine compositions is well known. Vaccines may comprise or may be formulated into a pharmaceutical composition comprising the vaccine and a pharmaceutically acceptable excipient. “Pharmaceutically acceptable” refers to the excipient that at the dosages and concentrations employed, will not cause unwanted or harmful effects in the subjects to which they are administered and include carrier, buffers, stabilizers, or other materials well known to those skilled in the art. The precise nature of the carrier or other material may depend on the route of administration, e.g., intramuscular, subcutaneous, oral, intravenous, cutaneous, intramucosal (e.g., gut), intranasal, or intraperitoneal routes. Liquid carriers such as water, petroleum, animal or vegetable oils, mineral oil or synthetic oil may be included. Physiological saline solution, dextrose or other saccharide solution or glycols such as ethylene glycol, propylene glycol or polyethylene glycol may be included. Exemplary viral formulations are the Adenovirus World Standard (Hoganson et al., 2002): 20 mM Tris pH 8, 25 mM NaCl, 2.5% glycerol; or 20 mM Tris, 2 mM MgCl₂, 25 mM NaCl, sucrose 10% w/v, polysorbate-80 0.02% w/v; or 10-25 mM citrate buffer pH 5.9-6.2, 4-6% (w/w) hydroxypropyl-beta-cyclodextrin (HBCD), 70-100 mM NaCl, 0.018-0.035% (w/w) polysorbate-80, and optionally 0.3-0.45% (w/w) ethanol. Many other buffers can be used, and examples of suitable formulations for the storage and for pharmaceutical administration of purified pharmaceutical preparations are known.

The vaccine may comprise one or more adjuvants. Suitable adjuvants include QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59. Other adjuvants that may be used include lectins, growth factors, cytokines and lymphokines such as alpha-interferon, gamma interferon, platelet derived growth factor (PDGF), granulocyte-colony stimulating factor (gCSF), granulocyte macrophage colony stimulating factor (gMCSF), tumor necrosis factor (TNF), epidermal growth factor (EGF), IL-1, IL-2, IL-4, IL-6, IL-8, IL-10, IL-12 or TLR agonists.

“Adjuvant” and “immune stimulant” are used interchangeably herein and are defined as one or more substances that cause stimulation of the immune system. In this context, an adjuvant is used to enhance an immune response to the vaccines described herein.

The disclosed methods can be used to treat any form of prostate cancer in a subject. The disclosed methods can treat, for example, a localized prostate adenocarcinoma, a relapsed prostate cancer, a refractory prostate cancer, a metastatic prostate cancer, a castration resistant prostate cancer, or any combination thereof. In some embodiments, the prostate cancer is an adenocarcinoma. In some embodiments, the prostate cancer is a metastatic prostate cancer. In some embodiments, the prostate cancer has metastasized to rectum, lymph node, or bone, or any combination thereof. In some embodiments, the prostate cancer is a relapsed or a refractory prostate cancer. In some embodiments, the prostate cancer is a castration resistant prostate cancer. In some embodiments, the prostate cancer is sensitive to an androgen deprivation therapy. In some embodiments, the prostate cancer is insensitive to the androgen deprivation therapy.

In some embodiments, the subject is treatment naïve. In some embodiments, the subject has received androgen deprivation therapy. In some embodiments, the subject has an elevated level of prostate specific antigen (PSA).

Androgen deprivation therapies include abiraterone, ketoconazole, enzalutamide, galeterone, ARN-509, and orteronel (TAK-700), or prostatectomy.

The methods of treatment can comprise administering any of the disclosed vaccines in combination with at least one additional cancer therapeutic agent for treating prostate cancer. The additional cancer therapeutic agent may be a chemotherapy, an androgen deprivation therapy, radiation therapy, targeted therapy, a checkpoint inhibitor, or any combination thereof. Any of the disclosed vaccines can also be used in combination with a surgery.

Exemplary chemotherapeutic agents include alkylating agents; nitrosoureas; antimetabolites; antitumor antibiotics; plant alkyloids; taxanes; hormonal agents; and miscellaneous agents, such as busulfan, carboplatin, chlorambucil, cisplatin, cyclophosphamide, dacarbazine, ifosfamide, mechlorethamine hydrochloride, melphalan, procarbazine, thiotepa, uracil mustard, 5-fluorouracil, 6-mercaptopurine, capecitabine, cytosine arabinoside, floxuridine, fludarabine, gemcitabine, methotrexate, thioguanine, dactinomycin, daunorubicin, doxorubicin, idarubicin, mitomycin-C, mitoxantrone, vinblastine, vincristine, vindesine, vinorelbine, paclitaxel, and docetaxel.

Exemplary androgen deprivation therapies include abiraterone acetate, ketoconazole, enzalutamide, galeterone, ARN-509 and orteronel (TAK-700) and surgical removal of the testicles.

Radiation therapy may be administered using various methods, including external-beam therapy, internal radiation therapy, implant radiation, stereotactic radiosurgery, systemic radiation therapy, radiotherapy and permanent or temporary interstitial brachytherapy. External-beam therapy involves three-dimensional, conformal radiation therapy where the field of radiation is designed, local radiation (e.g., radiation directed to a preselected target or organ), or focused radiation. Focused radiation may be selected from stereotactic radiosurgery, fractionated stereotactic radiosurgery, or intensity-modulated radiation therapy. Focused radiation may have particle beam (proton), cobalt-60 (photon) linear accelerator (x-ray) as a radiation source (see e.g. WO 2012/177624). “Brachytherapy,” refers to radiation therapy delivered by a spatially confined radioactive material inserted into the body at or near a tumor or other proliferative tissue disease site, and includes exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner include both solids and liquids. The radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material may also be a fluid made from any solution of radionuclide(s), e.g., a solution of I-125 or 1-131, or a radioactive fluid can be produced using a slurry of a suitable fluid containing small particles of solid radionuclides, such as Au-198, Y-90. The radionuclide(s) may be embodied in a gel or radioactive micro spheres.

Targeted therapies include anti-androgen therapies, inhibitors of angiogenesis such as bevacizumab, anti-PSA, or anti-PSMA antibodies or vaccines enhancing immune responses to PSA or PSMA.

Exemplary checkpoint inhibitors are antagonists of PD-1, PD-L1, PD-L2, VISTA, BTNL2, B7-H3, B7-H4, HVEM, HHLA2, CTLA-4, LAG-3, TIM-3, BTLA, CD160, CEACAM-1, LAIRI, TGFβ, IL-10, Siglec family protein, KIR, CD96, TIGIT, NKG2A, CD112, CD47, SIRPA or CD244. “Antagonist” refers to a molecule that, when bound to a cellular protein, suppresses at least one reaction or activity that is induced by a natural ligand of the protein. A molecule is an antagonist when the at least one reaction or activity is suppressed by at least about 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist. Antagonist may be an antibody, a soluble ligand, a small molecule, a DNA, or RNA such as siRNA. Exemplary antagonists of checkpoint inhibitors are described in U.S. Pat. Publ. No. 2017/0121409.

In some embodiments, one or more vaccines are administered in combination with a CTLA-4 antibody, a CTLA4 ligand, a PD-1 axis inhibitor, a PD-L1 axis inhibitor, a TLR agonist, a CD40 agonist, an OX40 agonist, hydroxyurea, ruxolitinib, fedratinib, a 41BB agonist, aa CD28 agonist, a STING antagonist, a RIG-1 antagonist, TCR-T therapy, CAR-T therapy, FLT3 ligand, aluminum sulfate, BTK inhibitor, CD38 antibody, CDK inhibitor, CD33 antibody, CD37 antibody, CD25 antibody, GM-CSF inhibitor, IL-2, IL-15, IL-7, CD3 redirection molecules, pomalimib, IFNγ, IFNα, TNFα, VEGF antibody, CD70 antibody, CD27 antibody, BCMA antibody or GPRC5D antibody, any combination thereof.

In some embodiments, the checkpoint inhibitor is ipilimumab, cetrelimab, pembrolizumab, nivolumab, sintilimab, cemiplimab, toripalimab, camrelizumab, tislelizumab, dostralimab, spartalizumab, prolgolimab, AK-105, HLX-10, balstilimab, MEDI-0680, HX-008, GLS-010, BI-754091, genolimzumab, AK-104, MGA-012, F-520, 609A, LY-3434172, AMG-404, SL-279252, SCT-I10A, RO-7121661, ICTCAR-014, MEDI-5752, CS-1003, XmAb-23104, Sym-021, LZM-009, hAB21, BAT-1306, MGD-019, JTX-4014, budigalimab, XmAb-20717, AK-103, MGD-013, IBI-318, sasanlimab, CC-90006, avelumab, atezolizumab, durvalumab, CS-1001, bintrafusp alpha, envafolimab, CX-072, GEN-1046, GS-4224, KL-A167, BGB-A333, SHR-1316, CBT-502, IL-103, KN-046, ZKAB-001, CA-170, TG_1501, LP-002, INCB-86550, ADG-104, SHR-1701, BCD-135, IMC-001, MSB-2311, FPT-155, FAZ-053, HLX-20, iodapolimab, FS-118, BMS-986189, AK-106, MCLA-145, IBI-318 or CK-301, or any combination thereof.

In some embodiments, one or more vaccines are administered in combination with ipilimumab, cetrelimab, pembrolizumab, nivolumab, sintilimab, cemiplimab, toripalimab, camrelizumab, tislelizumab, dostralimab, spartalizumab, prolgolimab, balstilimab, budigalimab, sasanlimab, avelumab, atezolizumab, durvalumab, envafolimab or iodapolimab, or any combination thereof.

### Methods for Monitoring Responsiveness to a Therapeutic Agent

Also disclosed are methods for monitoring responsiveness of a subject having prostate cancer to a therapeutic agent, the method comprising:

(a) evaluating expression of one or more prostate cancer biomarkers, wherein the one or more prostate cancer biomarkers comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, ROR1, FGF8, NKX2-2, EDIL3, RELN, FGF9, AKR1C4, CLUL1, KISSIR, CYP3A5, CYP17A1, SFRP4, HNF1A, CALCR, SYP, MSLN, or combinations thereof;

(b) administering a therapeutic agent to the subject; and

(c) evaluating the expression of the one or more prostate cancer biomarkers evaluated in step (a), wherein a decrease in the expression of the one or more prostate cancer biomarkers compared to the expression in step (a) is indicative of responsiveness to the therapeutic agent.

The sample from the subject from which the prostate cancer biomarkers is evaluated can comprise any biological sample known to contain or suspected of containing tumor material including, for example, a prostate cancer tissue sample or other types of materials containing cancer cells or biological derivatives from cancer cells (exosomes, apoptotic modies, circulating nucleic acids, etc.). In some embodiments, the sample is a plasma sample. In some aspects, the sample is from plasma exosomes. In some embodiments, the sample is a blood sample.

The one or more prostate cancer biomarkers can comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, or combinations thereof. In some embodiments, the one or more prostate cancer biomarkers are from a plasma sample. In some aspects, the one or more prostate cancer biomarkers are from plasma exosomes.

The one or more prostate cancer biomarkers can comprise: HPN, ROR1, FLNC, GPR39, FGF8, NKX2-2, MUC1, NKX3-1, EDIL3, LGR5, FGFR4, STEAP1, ATF3, RELN, UGT2B17, KLK3, C9orf152, GNMT, METTL7A, FGF9, SPDEF, FOXA1, AKR1C4, GREB1, CLUL1, TMEFF2, HOXB13, KLK2, NPY, GRHL2, STEAP2, THBS2, KISSIR, KRT8, TNFRSF19, CYP3A5, KLK4, IDO1, FOLH1, NROB1, EPHA3, CYP17A1, SFRP4, KRT18, TSPAN1, HNF1A, ADAMTS15, ACPP, CALCR, SYP, AZGP1, AR, ARv3, MSLN, TMPRSS2:ERG, and combinations thereof. In some embodiments, the one or more prostate cancer biomarkers are from a blood sample.

The presence of the one or more prostate cancer biomarkers can be evaluated by, for example, PCR, qPCR, various forms of nucleic acid sequencing (including but not limited to Illumina, Ion Torrent, Pacific Bioscience, Oxford Nanopore platforms), and various hybridization based approaches (including not limited to Affymetrix Gene Chip or Nanostring platforms). In some embodiments, the presence of the one or more prostate cancer biomarkers is evaluated by qPCR. In some aspects, the methods further comprise, prior to performing the qPCR, extracting RNA from the sample from the subject and synthesizing cDNA from the extracted RNA.

The expression of the one or more prostate cancer biomarkers detected in step (a) is the baseline expression of the cancer biomarker. Evaluating the expression of the one or more prostate cancer biomarkers after administering the therapeutic agent (step (c)) provides an indication of responsiveness/therapeutic efficacy. For example, a decrease in expression of the one or more prostate cancer biomarkers after administering the therapeutic agent compared to the expression prior to administering the therapeutic agent is indicative of responsiveness to the therapeutic agent.

Suitable therapeutic agents include any of the prostate cancer vaccines and additional agents disclosed above including, for example, surgery, chemotherapy, androgen deprivation therapy, radiation therapy, targeted therapy, checkpoint inhibitor, or any combination thereof

Methods for Preparing a cDNA from a Subject with Prostate Cancer Useful for Analyzing an Expression of Prostate Cancer Neoantigens

Also provided are methods for preparing a cDNA from a subject with prostate cancer useful for analyzing an expression of prostate cancer neoantigens, the method comprising:
(a) extracting RNA from a sample from the subject;
(b) producing amplified cDNA from the RNA extracted in step (a) by:
   (i) reverse transcribing the extracted RNA to produce the cDNA, and
   (ii) amplifying the cDNA; and
(c) analyzing the amplified cDNA produced in step (b) for one or more prostate cancer neoantigens, wherein the cDNA encodes an amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof.

The sample from the subject can comprise any biological sample known to contain or suspected of containing tumor material including, for example, a prostate cancer tissue sample or other types of materials containing cancer cells or biological derivatives from cancer cells (exosomes, apoptotic modies, circulating nucleic acids, etc.). In some embodiments, the sample from the subject is a prostate cancer tissue sample.

In some embodiments, the cDNA encodes an amino acid sequence of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, fragments of the preceding sequences, or any combination thereof.

In some embodiments, the cDNA encodes an amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, 223, fragments of the preceding sequences, or any combination thereof.

In some embodiments, the cDNA encodes an amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, and 223.

### Exemplary Polypeptide and Polynucleotide Sequences for the Prostate Cancer Vaccines

The prostate cancer vaccine can comprise two or more polypeptides selected from the group consisting of:
AS18 comprising the amino acid sequence WKFEMSYTVGGPPPHVHARPRHWKTDR (SEQ ID NO: 275);
P87 comprising the amino acid sequence YEAGMTLGGKILFFLFLLLPLSPFSLIF (SEQ ID NO: 381);
AS55 comprising the amino acid sequence DGHSYTSKVNCLLLQDGFHGCVSITGAAGRRNLSIFLFLMLCKLEFHAC (SEQ ID NO: 333);
AS57 comprising the amino acid sequence TGGKSTCSAPGPQSLPSTPFSTYPQWVILITEL (SEQ ID NO: 337);
AS15 comprising the amino acid sequence VLRFLDLKVRYLHS (SEQ ID NO: 269);
AS7 comprising the amino acid sequence DYWAQKEKGSSSFLRPSC (SEQ ID NO: 253);
AS43 comprising the amino acid sequence VPFRELKNVSVLEGLRQGRLGGPCSCHCPRPSQARLTPVDVAGPFLCLGDPGLFPPVKSSI (SEQ ID NO: 309);
AS51 comprising the amino acid sequence GMECTLGQVGAPSPRREEDGWRGGHSRFKADVPAPQGPCWGGQPGSAPSSAPPEQSLL D (SEQ ID NO: 325);
AS16 comprising the amino acid sequence GNTTLQQLGEASQAPSGSLIPLRLPLLWEVRG (SEQ ID NO: 271);
AS41 comprising the amino acid sequence EAFQRAAGEGGPGRGGARRGARVLQSPFCRAGAGEWLGHQSLR (SEQ ID NO: 305);
AS6 comprising the amino acid sequence DYWAQKEKISIPRTHLC (SEQ ID NO: 251);
AS3 comprising the amino acid sequence VAMMVPDRQVHYDFGL (SEQ ID NO: 245);
AS11 comprising the amino acid sequence VPFRELKNQRTAQGAPGIHHAASPVAANLCDPARHAQHTRIPCGAGQVRAGRGPEAGG GVLQPQRPAPEKPGCPCRRGQPRLHTVKMWRA (SEQ ID NO: 261);
AS13 comprising the amino acid sequence KRSFAVTERII (SEQ ID NO: 265);
AS47 comprising the amino acid sequence FKKFDGPCGERGGGRTARALWARGDSVLTPALDPQTPVRAPSLTRAAAAV (SEQ ID NO: 317);
AS8 comprising the amino acid sequence LVLGVLSGHSGSRL (SEQ ID NO: 255);
AS19 comprising the amino acid sequence QWQHYHRSGEAAGTPLWRPTRN (SEQ ID NO: 277);
AS37 comprising the amino acid sequence CHLFLQPQVGTPPPHTASARAPSGPPHPHESCPAGRRPARAAQTCARRQHGLPGCEEAGT ARVPSLHLHLHQAALGAGRGRGWGEACAQVPPSRG (SEQ ID NO: 297);
AS23 comprising the amino acid sequence KIQNKNCPD (SEQ ID NO: 285);
MS1 comprising the amino acid sequence HYKLIQQPISLFSITDRLHKTFSQLPSVHLCSITFQWGHPPIFCSTNDICVTANFCISVTFLKP CFLLHEASASQ (SEQ ID NO: 437);
MS3 comprising the amino acid sequence RTALTHNQDFSIYRLCCKRGSLCHASQARSPAFPKPVRPLPAPITRITPQLGGQSDSSQPLL TTGRPQGWQDQALRHTQQASPASCATITIPIHSAALGDHSGDPGPAWDTCPPLPLTTLIPR APPPYGDSTARSWPSRCGPLG (SEQ ID NO: 439);
MS6 comprising the amino acid sequence YAYKDFLWCFPFSLVFLQEIQICCHVSCLCCICCSTRICLGCLLELFLSRALRALHVLWNG FQLHCQ (SEQ ID NO: 442);
MS8 comprising the amino acid sequence TMPAILKLQKNCLLSL (SEQ ID NO: 444); and
P82 comprising the amino acid sequence YEAGMTLGEKFRVGNCKHLKMTRP (SEQ ID NO: 379),
and fragments thereof.

In some embodiments, the prostate cancer vaccine can comprise one or more polypeptides selected from the group consisting of:
P16 comprising the amino acid sequence GVPGDSTRRAVRRMNTF (SEQ ID NO: 343);
FUS1 comprising the amino acid sequence CGASACDVSLIAMDSA (SEQ ID NO: 211);
P22 comprising the amino acid sequence SLYHREKQLIAMDSAI (SEQ ID NO: 349);
FUS2 comprising the amino acid sequence TEYNQKLQVNQFSESK (SEQ ID NO: 213);
FUS3 comprising the amino acid sequence TEISCCTLSSEENEYLPRPEWQLQ (SEQ ID NO: 215);
FUS6 comprising the amino acid sequence CEERGAAGSLISCE (SEQ ID NO: 221);
FUS5 comprising the amino acid sequence NSKMALNSEALSVVSE (SEQ ID NO: 219);
FUS8 comprising the amino acid sequence WGMELAASRRFSWDHHSAGGPPRVPSVRSGAAQVQPKDPLPLRTLAGCLARTAHLRPG AESLPQPQLHCT (SEQ ID NO: 225);
FUS15 comprising the amino acid sequence HVVGYGHLDTSGSSSSSSWP (SEQ ID NO: 345);
P35 comprising the amino acid sequence NSKMALNSLNSIDDAQLTRIAPPRSHCCFWEVNAP (SEQ ID NO: 353);
FUS19 comprising the amino acid sequence KMHFSLKEHPPPPCPP (SEQ ID NO: 235); and
FUS7 comprising the amino acid sequence LWFQSSELSPTGAPWPSRRPTWRGTTVSPRTATSSARTCCGTKWPSSQEAALGLGSGLLR FSCGTAAIR (SEQ ID NO: 223),
and fragments thereof.

The prostate cancer vaccine can comprise two or more polypeptides selected from the group consisting of:
P16 comprising the amino acid sequence GVPGDSTRRAVRRMNTF (SEQ ID NO: 343);
FUS1 comprising the amino acid sequence CGASACDVSLIAMDSA (SEQ ID NO: 211);
P22 comprising the amino acid sequence SLYHREKQLIAMDSAI (SEQ ID NO: 349);
FUS2 comprising the amino acid sequence TEYNQKLQVNQFSESK (SEQ ID NO: 213);
FUS3 comprising the amino acid sequence TEISCCTLSSEENEYLPRPEWQLQ (SEQ ID NO: 215);
FUS6 comprising the amino acid sequence CEERGAAGSLISCE (SEQ ID NO: 221);
FUS5 comprising the amino acid sequence NSKMALNSEALSVVSE (SEQ ID NO: 219);
FUS8 comprising the amino acid sequence WGMELAASRRFSWDHHSAGGPPRVPSVRSGAAQVQPKDPLPLRTLAGCLARTAHLRPG AESLPQPQLHCT (SEQ ID NO: 225);
FUS15 comprising the amino acid sequence HVVGYGHLDTSGSSSSSSWP (SEQ ID NO: 345);
P35 comprising the amino acid sequence NSKMALNSLNSIDDAQLTRIAPPRSHCCFWEVNAP (SEQ ID NO: 353);
FUS19 comprising the amino acid sequence KMHFSLKEHPPPPCPP (SEQ ID NO: 235); and
FUS7 comprising the amino acid sequence LWFQSSELSPTGAPWPSRRPTWRGTTVSPRTATSSARTCCGTKWPSSQEAALGLGSGLLR FSCGTAAIR (SEQ ID NO: 223),
and fragments thereof.

The prostate cancer vaccine can comprise one or more polypeptides selected from the group consisting of:
AS18 comprising the amino acid sequence WKFEMSYTVGGPPPHVHARPRHWKTDR (SEQ ID NO: 275);
P87 comprising the amino acid sequence YEAGMTLGGKILFFLFLLLPLSPFSLIF (SEQ ID NO: 381);
AS55 comprising the amino acid sequence DGHSYTSKVNCLLLQDGFHGCVSITGAAGRRNLSIFLFLMLCKLEFHAC (SEQ ID NO: 333);
AS57 comprising the amino acid sequence TGGKSTCSAPGPQSLPSTPFSTYPQWVILITEL (SEQ ID NO: 337);
AS15 comprising the amino acid sequence VLRFLDLKVRYLHS (SEQ ID NO: 269);
AS7 comprising the amino acid sequence DYWAQKEKGSSSFLRPSC (SEQ ID NO: 253);
AS43 comprising the amino acid sequence VPFRELKNVSVLEGLRQGRLGGPCSCHCPRPSQARLTPVDVAGPFLCLGDPGLFPPVKSSI (SEQ ID NO: 309);
AS51 comprising the amino acid sequence GMECTLGQVGAPSPRREEDGWRGGHSRFKADVPAPQGPCWGGQPGSAPSSAPPEQSLL D (SEQ ID NO: 325);
AS16 comprising the amino acid sequence GNTTLQQLGEASQAPSGSLIPLRLPLLWEVRG (SEQ ID NO: 271);
AS41 comprising the amino acid sequence EAFQRAAGEGGPGRGGARRGARVLQSPFCRAGAGEWLGHQSLR (SEQ ID NO: 305);
AS6 comprising the amino acid sequence DYWAQKEKISIPRTHLC (SEQ ID NO: 251);
AS3 comprising the amino acid sequence VAMMVPDRQVHYDFGL (SEQ ID NO: 245);
AS11 comprising the amino acid sequence VPFRELKNQRTAQGAPGIHHAASPVAANLCDPARHAQHTRIPCGAGQVRAGRGPEAGG GVLQPQRPAPEKPGCPCRRGQPRLHTVKMWRA (SEQ ID NO: 261);
AS13 comprising the amino acid sequence KRSFAVTERII (SEQ ID NO: 265);
AS47 comprising the amino acid sequence FKKFDGPCGERGGGRTARALWARGDSVLTPALDPQTPVRAPSLTRAAAAV (SEQ ID NO: 317);
AS8 comprising the amino acid sequence LVLGVLSGHSGSRL (SEQ ID NO: 255);
AS19 comprising the amino acid sequence QWQHYHRSGEAAGTPLWRPTRN (SEQ ID NO: 277);
AS37 comprising the amino acid sequence CHLFLQPQVGTPPPHTASARAPSGPPHPHESCPAGRRPARAAQTCARRQHGLPGCEEAGT ARVPSLHLHLHQAALGAGRGRGWGEACAQVPPSRG (SEQ ID NO: 297);
AS23 comprising the amino acid sequence KIQNKNCPD (SEQ ID NO: 285);
MS1 comprising the amino acid sequence HYKLIQQPISLFSITDRLHKTFSQLPSVHLCSITFQWGHPPIFCSTNDICVTANFCISVTFLKP CFLLHEASASQ (SEQ ID NO: 437);
MS3 comprising the amino acid sequence RTALTHNQDFSIYRLCCKRGSLCHASQARSPAFPKPVRPLPAPITRITPQLGGQSDSSQPLL TTGRPQGWQDQALRHTQQASPASCATITIPIHSAALGDHSGDPGPAWDTCPPLPLTTLIPR APPPYGDSTARSWPSRCGPLG (SEQ ID NO: 439);
MS6 comprising the amino acid sequence YAYKDFLWCFPFSLVFLQEIQICCHVSCLCCICCSTRICLGCLLELFLSRALRALHVLWNG FQLHCQ (SEQ ID NO: 442);
MS8 comprising the amino acid sequence TMPAILKLQKNCLLSL (SEQ ID NO: 444); and
P82 comprising the amino acid sequence YEAGMTLGEKFRVGNCKHLKMTRP (SEQ ID NO: 379),
and fragments thereof.

In some embodiments, the prostate cancer vaccine can comprise one or more polypeptides selected from the group consisting of:
M84 comprising the amino acid sequence IARELHQFAFDLLIKSH (SEQ ID NO: 167);
M86 comprising the amino acid sequence QPDSFAALHSSLNELGE (SEQ ID NO: 171);
M10 comprising the amino acid sequence FVQGKDWGLKKFIRRDF (SEQ ID NO: 19);
M12 comprising the amino acid sequence FVQGKDWGVKKFIRRDF (SEQ ID NO: 23); and
FR1 comprising the amino acid sequence QNLQNGGGSRSSATLPGRRRRRWLRRRRQPISVAPAGPPRRPNQKPNPPGGARCVIMRPT WPGTSAFT (SEQ ID NO: 177),
and fragments thereof.

The prostate cancer vaccine can comprise one or more polynucleotides selected from the group consisting of:
the polynucleotide sequence of TGGAAATTCGAGATGAGCTACACGGTGGGTGGCCCGCCTCCCCATGTTCATGCTAGA CCCAGGCATTGGAAAACTGATAGA (SEQ ID NO: 276) (encoding AS18);
the polynucleotide sequence of TATGAAGCAGGGATGACTCTGGGAGGTAAGATACTTTTCTTTCTCTTCCTCCTCCTTC CTCTCTCCCCCTTCTCCCTCATTTTC (SEQ ID NO: 382) (encoding P87);
the polynucleotide sequence of GATGGCCACTCCTACACATCCAAGGTGAATTGTTTACTCCTTCAAGATGGGTTCCATG GCTGTGTGAGCATCACCGGGGCAGCTGGAAGAAGAAACCTGAGCATCTTCCTGTTCT TGATGCTGTGCAAATTGGAGTTCCATGCTTGT (SEQ ID NO: 334) (encoding AS55);
the polynucleotide sequence of ACAGGGGGCAAAAGCACCTGCTCGGCTCCTGGCCCTCAGTCTCTCCCCTCCACTCCA TTCTCCACCTACCCACAGTGGGTCATTCTGATCACCGAACTG (SEQ ID NO: 338) (encoding AS57);
the polynucleotide sequence of GTGCTGCGCTTTCTGGACTTAAAGGTGAGATACCTGCACTCT (SEQ ID NO: 270) (encoding AS15);
the polynucleotide sequence of GACTACTGGGCTCAAAAGGAGAAGGGATCATCTTCATTCCTGCGACCATCCTGT (SEQ ID NO: 254) (encoding AS7);
the polynucleotide sequence of GTGCCCTTCCGGGAGCTCAAGAACGTGAGTGTCCTGGAGGGGCTCCGTCAAGGCCGG CTTGGGGGTCCCTGTTCATGTCACTGCCCAAGACCTTCCCAGGCCAGGCTCACGCCA GTGGATGTGGCAGGTCCCTTCTTGTGTCTGGGGGATCCTGGGCTGTTCCCCCCAGTCA AGAGCAGTATC (SEQ ID NO: 310) (encoding AS43);
the polynucleotide sequence of GGCATGGAGTGCACCCTGGGGCAGGTGGGTGCCCCGTCCCCTCGGAGGGAGGAGGA CGGTTGGCGTGGGGGCCACAGCCGATTCAAGGCTGATGTACCAGCACCGCAGGGAC CCTGCTGGGGTGGCCAACCTGGCTCTGCCCCCTCCTCAGCTCCTCCTGAACAGTCATT ATTAGAT (SEQ ID NO: 326) (encoding AS51);
the polynucleotide sequence of GGCAACACCACCCTCCAGCAGCTGGGTGAGGCCTCCCAGGCGCCCTCAGGCTCCCTC ATCCCTCTGAGGCTGCCTCTGCTCTGGGAAGTGAGGGGC ((SEQ ID NO: 272) (encoding AS16);
the polynucleotide sequence of GAGGCCTTCCAGAGGGCCGCTGGTGAGGGCGGCCCGGGCCGCGGTGGGGCACGGCG CGGTGCCAGGGTGTTGCAGAGCCCCTTTTGCAGGGCAGGAGCTGGGGAGTGGTTAGG ACATCAGTCCCTCAGG (SEQ ID NO: 306) (encoding AS41);
the polynucleotide sequence of GACTACTGGGCTCAAAAGGAGAAGATCAGCATCCCCAGAACACACCTGTGT (SEQ ID NO: 252) (encoding AS6);
the polynucleotide sequence of GTTGCTATGATGGTTCCTGATAGACAGGTTCATTATGACTTTGGATTG (SEQ ID NO: 246) (encoding AS3);
the polynucleotide sequence of GTGCCCTTCCGGGAGCTCAAGAACCAGAGAACAGCACAAGGGGCTCCTGGGATCCA CCACGCGGCTTCCCCCGTTGCTGCCAACCTCTGCGACCCGGCGAGACACGCACAGCA CACACGCATCCCCTGCGGCGCTGGCCAAGTGCGTGCTGGCCGAGGTCCCGAAGCAGG TGGTGGAGTACTACAGCCACAGAGGCCTGCCCCCGAGAAGCCTGGGTGTCCCTGCCG GAGAGGCCAGCCCAGGCTGCACACCGTGAAGATGTGGAGGGCG (SEQ ID NO: 262) (encoding AS11);
the polynucleotide sequence of AAGAGAAGTTTTGCTGTCACGGAGAGGATCATC (SEQ ID NO: 266) (encoding AS13);
the polynucleotide sequence of TTCAAGAAGTTCGACGGCCCTTGTGGTGAGCGCGGCGGCGGGCGCACGGCTCGAGCT CTGTGGGCGCGCGGCGACAGCGTCCTGACTCCTGCCCTCGACCCCCAGACCCCTGTC AGGGCGCCCTCCCTGACCCGAGCCGCAGCTGCCGTG (SEQ ID NO: 318) (encoding AS47);
the polynucleotide sequence of CTTGTACTTGGTGTATTGAGCGGGCACAGTGGCTCACGCCTA (SEQ ID NO: 256) (encoding AS8);
the polynucleotide sequence of CAGTGGCAGCACTACCACCGGTCAGGTGAGGCCGCAGGGACTCCCCTCTGGAGACCC ACAAGAAAC (SEQ ID NO: 278) (encoding AS19);
the polynucleotide sequence of TGCCACCTCTTCCTGCAGCCCCAGGTTGGCACCCCCCCCCCCCACACTGCCAGTGCTC GAGCCCCCAGTGGTCCACCCCACCCTCATGAAAGTTGCCCTGCAGGGCGAAGACCTG CGAGAGCTGCGCAGACATGTGCACGCCGACAGCACGGACTTCCTGGCTGTGAAGAG GCTGGTACAGCGCGTGTTCCCAGCCTGCACCTGCACCTGCACCAGGCCGCCCTCGGA GCAGGAAGGGGCCGTGGGTGGGGAGAGGCCTGTGCCCAAGTACCCCCCTCAAGAGG C (SEQ ID NO: 298) (encoding AS37);
the polynucleotide sequence of AAAATTCAGAATAAAAATTGTCCAGAC (SEQ ID NO: 286) (encoding AS23);
the polynucleotide sequence of CACTACAAATTAATTCAACAACCCATATCCCTCTTCTCCATCACTGATAGGCTCCATA AGACGTTCAGTCAGCTGCCCTCGGTCCATCTCTGCTCAATCACCTTCCAGTGGGGACA CCCGCCCATTTTCTGCTCAACAAATGATATCTGTGTCACGGCCAACTTCTGCATCTCG GTCACATTCCTTAAACCGTGCTTCCTCCTACATGAGGCATCTGCCTCACAG (SEQ ID NO: 448) (encoding MS1);
the polynucleotide sequence of AGGACCGCCCTGACACACAATCAGGACTTCTCTATCTACAGGCTCTGTTGCAAGAGG GGGTCCCTCTGCCACGCTTCCCAGGCCAGATCCCCGGCTTTCCCGAAGCCGGTCAGA CCTCTTCCTGCCCCCATCACCAGAATCACCCCCCAACTGGGGGGACAATCTGACTCG AGTCAACCCCTTCTCACTACGGGAAGACCTCAGGGGTGGCAAGATCAAGCTCTTAGA CACACCCAGCAAGCCAGTCCTGCCTCTTGTGCCACCATCACCATTCCCATCCACTCAG CTGCCCTTGGTGACCACTCCGGAGACCCTGGTCCAGCCTGGGACACCTGCCCGCCGC TGCCGCTCACTACCCTCATCCCCCGAGCTCCCCCGCCGTATGGAGACAGCACTGCCA GGTCCTGGCCCTCCCGCTGTGGGCCCCTCGGC (SEQ ID NO: 450) (encoding MS3);
the polynucleotide sequence of TATGCTTACAAGGACTTTCTCTGGTGTTTTCCTTTTTCTTTAGTTTTTCTCCAAGAGAT TCAAATCTGCTGCCATGTTAGCTGTCTTTGCTGTATCTGCTGTAGTACACGAATATGC CTTGGCTGTTTGCTTGAGCTTTTTCTATCCCGTGCTCTTCGTGCTCTTCATGTTCTTTG GAATGGCTTTCAACTTCATTGTCAA (SEQ ID NO: 453) (encoding MS6);
the polynucleotide sequence of ACCATGCCTGCTATTTTAAAGTTACAGAAGAATTGTCTTCTCTCCTTA (SEQ ID NO: 455) (encoding MS8); and
the polynucleotide sequence of TATGAAGCAGGGATGACTCTGGGAGAAAAATTCCGGGTTGGCAATTGCAAGCATCTC AAAATGACCAGACCC (SEQ ID NO: 380) (encoding P82); and fragments thereof.

The prostate cancer vaccine can comprise one or more polynucleotides selected from the group consisting of:
the polynucleotide sequence of GGAGTTCCAGGAGATTCAACCAGGAGAGCAGTGAGGAGAATGAATACCTTC (SEQ ID NO: 344) (encoding P16);
the polynucleotide sequence of TGCGGGGCCTCTGCCTGTGATGTCTCCCTCATTGCTATGGACAGTGCT (SEQ ID NO: 212) (encoding FUS1);
the polynucleotide sequence of TCCCTCTACCACCGGGAGAAGCAGCTCATTGCTATGGACAGTGCTATC (SEQ ID NO: 350) (encoding P22);
the polynucleotide sequence of ACCGAATACAACCAGAAATTACAAGTGAATCAATTTAGTGAATCCAAA (SEQ ID NO: 214) (encoding FUS2);
the polynucleotide sequence of ACAGAAATTTCATGTTGCACCCTGAGCAGTGAGGAGAATGAATACCTTCCAAGACCA GAGTGGCAGCTCCAG (SEQ ID NO: 216) (encoding FUS3);
the polynucleotide sequence of TGTGAGGAGCGCGGCGCGGCAGGAAGCCTTATCAGTTGTGAG (SEQ ID NO: 222) (encoding FUS6);
the polynucleotide sequence of AACAGCAAGATGGCTTTGAACTCAGAAGCCTTATCAGTTGTGAGTGAG (SEQ ID NO: 220) (encoding FUS5);
the polynucleotide sequence of TGGGGGATGGAGTTGGCAGCGTCTCGGAGGTTCTCCTGGGACCACCACTCCGCCGGG GGGCCGCCCAGAGTGCCAAGCGTCCGATCCGGCGCCGCCCAAGTGCAGCCCAAGGA CCCGCTCCCGCTCCGCACCCTGGCAGGCTGCCTAGCCAGGACTGCGCACCTGCGCCC TGGGGCGGAGTCCTTACCCCAACCCCAGCTTCACTGCACA (SEQ ID NO: 226) (encoding FUS8);
the polynucleotide sequence of CACGTGGTGGGCTATGGCCACCTTGATACTTCCGGGTCATCCTCCTCCTCCTCCTGGC CC (SEQ ID NO: 346) (encoding FUS15);
the polynucleotide sequence of AACAGCAAGATGGCTTTGAACTCATTAAACTCCATTGATGATGCACAGTTGACAAGA ATTGCCCCTCCAAGATCTCATTGCTGTTTCTGGGAAGTAAACGCTCCT (SEQ ID NO: 354) (encoding P35);
the polynucleotide sequence of AAAATGCACTTCTCCCTCAAGGAGCACCCACCGCCCCCTTGCCCGCCT (SEQ ID NO: 236) (encoding FUS19); and
the polynucleotide sequence of CTGTGGTTCCAGAGCAGTGAGCTGTCCCCGACGGGAGCGCCATGGCCCAGCCGCCGC CCGACGTGGAGGGGGACGACTGTCTCCCCGCGTACCGCCACCTCTTCTGCCCGGACC TGCTGCGGGACAAAGTGGCCTTCATCACAGGAGGCGGCTCTGGGATTGGGTTCCGGA TTGCTGAGATTTTCATGCGGCACGGCTGCCATACGG (SEQ ID NO: 224) (encoding FUS7),
and fragments thereof.

The prostate cancer vaccine can comprise one or more polynucleotides selected from the group consisting of:
the polynucleotide sequence of GGAGTTCCAGGAGATTCAACCAGGAGAGCAGTGAGGAGAATGAATACCTTC (SEQ ID NO: 344) (encoding P16);
the polynucleotide sequence of TGCGGGGCCTCTGCCTGTGATGTCTCCCTCATTGCTATGGACAGTGCT (SEQ ID NO: 212) (encoding FUS1);
the polynucleotide sequence of TCCCTCTACCACCGGGAGAAGCAGCTCATTGCTATGGACAGTGCTATC (SEQ ID NO: 350) (encoding P22);
the polynucleotide sequence of ACCGAATACAACCAGAAATTACAAGTGAATCAATTTAGTGAATCCAAA (SEQ ID NO: 214) (encoding FUS2);
the polynucleotide sequence of ACAGAAATTTCATGTTGCACCCTGAGCAGTGAGGAGAATGAATACCTTCCAAGACCA GAGTGGCAGCTCCAG (SEQ ID NO: 216) (encoding FUS3);
the polynucleotide sequence of TGTGAGGAGCGCGGCGCGGCAGGAAGCCTTATCAGTTGTGAG (SEQ ID NO: 222) (encoding FUS6);
the polynucleotide sequence of AACAGCAAGATGGCTTTGAACTCAGAAGCCTTATCAGTTGTGAGTGAG (SEQ ID NO: 220) (encoding FUS5);
the polynucleotide sequence of TGGGGGATGGAGTTGGCAGCGTCTCGGAGGTTCTCCTGGGACCACCACTCCGCCGGG GGGCCGCCCAGAGTGCCAAGCGTCCGATCCGGCGCCGCCCAAGTGCAGCCCAAGGA CCCGCTCCCGCTCCGCACCCTGGCAGGCTGCCTAGCCAGGACTGCGCACCTGCGCCC TGGGGCGGAGTCCTTACCCCAACCCCAGCTTCACTGCACA (SEQ ID NO: 226) (encoding FUS8);
the polynucleotide sequence of CACGTGGTGGGCTATGGCCACCTTGATACTTCCGGGTCATCCTCCTCCTCCTCCTGGC CC (SEQ ID NO: 346) (encoding FUS15);
the polynucleotide sequence of AACAGCAAGATGGCTTTGAACTCATTAAACTCCATTGATGATGCACAGTTGACAAGA ATTGCCCCTCCAAGATCTCATTGCTGTTTCTGGGAAGTAAACGCTCCT (SEQ ID NO: 354) (encoding P35);
the polynucleotide sequence of AAAATGCACTTCTCCCTCAAGGAGCACCCACCGCCCCCTTGCCCGCCT (SEQ ID NO: 236) (encoding FUS19); and
the polynucleotide sequence of CTGTGGTTCCAGAGCAGTGAGCTGTCCCCGACGGGAGCGCCATGGCCCAGCCGCCGC CCGACGTGGAGGGGGACGACTGTCTCCCCGCGTACCGCCACCTCTTCTGCCCGGACC TGCTGCGGGACAAAGTGGCCTTCATCACAGGAGGCGGCTCTGGGATTGGGTTCCGGA TTGCTGAGATTTTCATGCGGCACGGCTGCCATACGG (SEQ ID NO: 224) (encoding FUS7).

### EXAMPLES

The following examples are provided to further describe some of the embodiments disclosed herein. The examples are intended to illustrate, not to limit, the disclosed embodiments.

### Example 1 General Methods

### Peptide Synthesis

Peptides were synthesized by New England Peptide with purity >80%. The lyophilized peptides were solubilized in 100% DMSO.

### In Vitro Immunogenicity Assessment of Neoantigens (“Patient PBMC Restimulation Assay”)

PBMCs from human patients with metastatic castrate-resistant prostate cancer were thawed using media (RPMI 1640 supplemented with Glutamax, 10% HI FBS, and 1× Sodium Pyruvate). Cells were counted and plated in a 96 well round bottom microplate at a concentration of 250,000 viable cells per well. Lyophilized peptides were solubilized in 100% DMSO and diluted in media to 10 μg/mL. Neoantigen peptides were added in equal volume to PBMCs for a final concentration of 5 μg/mL. CEF Peptide Pool “Plus” (Cellular Technologies, Ltd.) was utilized as a positive control and DMSO at the same final concentration as the experimental peptides was utilized as a negative control. Human IL-15 (Peprotech) was added to all wells at final concentration of 10 ng/mL.

Plates were incubated at 37° C. (5% CO₂) for a total of 13 days. Media was refreshed every 2 days with IL-15 (10 ng/mL final concentration) and IL-2 (R&D systems, 10 IU/mL final concentration). On day 12, PBMCs were re-stimulated with identical experimental peptides or controls, at same concentration as peptide stimulation on Day 1. After 1-hour incubation, protein Inhibitor Cocktail (eBioscience) was added to every well and plate was incubated overnight.

On day 13, cells were stained for intracellular flow cytometry analysis. The cells were washed with PBS and stained with Live/Dead Fixable Aqua Dead Cell stain (Thermo-Fisher). Following the live/dead stain, cells were blocked using Biotin-Free Fc Receptor Blocker (Accurate Chemical & Scientific Corp). Extracellular cellular flow panel (1 μL/antibody per well in 50 μL) consisted of CD3 PerCP-Cy5.5 (Biolegend), CD4 BV421 (Biolegend), and CD8 APC-Cy7 (Biolegend). After extracellular staining, cells were fixed using Foxp3/Transcription Factor Staining Buffer Set (eBioscience) and stained for intracellular proteins (1:50 dilution) using TNFα FITC (R&D Systems) and IFNγ BV785 (Biolegend). Cells were washed and resuspended in stain buffer and analyzed using a BD Celesta flow cytometer.

Flow cytometry cell staining analysis was completed using FlowJo v10. Cells were gated on live, singlet, CD3+ cells. The CD8+ T cells were analyzed for TNFα/IFNγ expression and the frequency of double positive TNFα/IFNγ CD8+ T cells was recorded. Responses were assessed to be positive when the frequency of double positive TNFα/IFNγ CD8+ T cells due to stimulation with an experimental peptide was increased greater than or equal to 2-fold over the DMSO only negative control for that patient. Peptides were analyzed in 1 to 7 patient samples.

### In Vitro Immunogenicity Assessment of Neoantigens (“Exogenous Autologous Normal Donor Restimulation Assay”)

CD1c+ Dendritic Cells (DC) isolated from human normal PBMCs were thawed using media (IMDM (Gibco) supplemented with glutamine, HEPES, 5% human serum (Sigma), and 1× Pen-Strep). DC cells were resuspended in media supplemented with IL-4 (Peprotech, 80 ng/mL) and GM-CSF (Gibco, 80 ng/mL), plated in 6 well microplates, and rested overnight at 37° C. (5% CO₂). The following day, DC cells were counted and plated in a 96 well round bottom microplate at a concentration of 30,000 viable cells per well. Lyophilized peptides (15-mer overlapping peptides) were solubilized in 100% DMSO and pooled by neoantigen to between 5 mg/mL and 20 mg/mL. Neoantigen peptides pools were added to DCs for a final concentration of 2.5 μg/mL to 10 μg/mL and rested for 2 hours at 37° C. (5% CO₂). CEF Peptide Pool “Plus” (Cellular Technologies, Ltd.) was utilized as a positive control and DMSO at the same final concentration as the experimental peptides was utilized as a negative control. After 2 hours, DC cells were irradiated with 50 gray of ionizing radiation. Autologous CD3+ Pan-T cells isolated from human normal PBMCs were thawed using media. Following irradiation, autologous Pan-T cells were added to the irradiated DCs at 300,000 viable cells per well. Human IL-15 (Peprotech) was added to all wells at final concentration of 10 ng/mL.

Plates were incubated at 37° C. (5% CO₂) for a total of 12 days. Media was refreshed every 2-3 days with IL-15 (10 ng/mL final concentration) and IL-2 (R&D systems, 10 IU/mL final concentration). On day 11 cells were re-stimulated with identical experimental peptide pools or controls, at same concentration as peptide stimulation on Day 1. Protein Inhibitor Cocktail (eBioscience) was added to every well and plate was incubated overnight at 37° C. (5% CO₂).

On day 12, cells were stained for intracellular flow cytometry analysis. The cells were washed with PBS and stained with Live/Dead Fixable Aqua Dead Cell stain (Thermo-Fisher). Following the live/dead stain, cells were blocked using Biotin-Free Fc Receptor Blocker (Accurate Chemical & Scientific Corp). Extracellular cellular flow panel (1 μL/antibody per well in 50 μL) consisted of CD3 PerCP-Cy5.5 (Biolegend), CD4 BV421 (Biolegend), and CD8 APC-Cy7 (Biolegend). After extracellular staining, cells were fixed using Foxp3/Transcription Factor Staining Buffer Set (eBioscience) and stained for intracellular proteins (1:50 dilution) using TNFα FITC (R&D Systems) and IFNγ BV785 (Biolegend). Cells were washed and resuspended in stain buffer and analyzed using a BD Celesta flow cytometer.

Flow cytometry cell staining analysis was completed using FlowJo v10. Cells were gated on live, singlet, CD3+ cells. The CD8+ and CD4+ T cells were analyzed for TNFα/IFNγ expression and the frequency of double positive TNFα/IFNγ CD8+ and the frequency of double positive TNFα/IFNγ CD4+ T cells were recorded. Responses were assessed to be positive when the frequency of double positive TNFα/IFNγ CD8+ or TNFα/IFNγ CD4+ T cells due to stimulation with an experimental peptide pool was increased greater than or equal to 3-fold over the DMSO only negative control for that donor and at least 0.01%.

### In Vitro Endogenous Immunogenicity Assessment of Neoantigens (“Endogenous Autologous Normal Donor Restimulation Assay”)

CD1c+ Dendritic Cells (DC) isolated from human normal PBMCs were thawed using media (IMDM (Gibco) supplemented with glutamine, HEPES, 5% human serum (Sigma), and 1× Pen-Strep). DC cells were resuspended in media supplemented with IL-4 (Peprotech, 80 ng/mL) and GM-CSF (Gibco, 80 ng/mL), plated in 6 well microplates, and rested overnight at 37° C. (5% CO₂). The following day, DC cells were counted and plated in a 96 well round bottom microplate at a concentration of 30,000 viable cells per well. Ad5 vectors (Vector Biolabs) were dilute in media to an MOI (Multiplicity Of Infection) of 5000 based on Plaque Forming Units. Ad5 vectors for the CEF pool and a “null” were used as controls. DCs were transduced with Ad5 vectors overnight at 37° C. (5% CO₂). The following day, the Ad5 vectors were washed off the plate by three sequential centrifugation/aspiration steps using sterile Phosphate Buffered Saline. After the final wash, transduced DCs were resuspended in 100 μL media. Autologous CD3+ Pan-T cells isolated from human normal PBMCs were thawed using media. Pan-T cells were added to the irradiated DCs at 300,000 viable cells per well (100 μL/well). Human IL-15 (Peprotech) was added to all wells at final concentration of 10 ng/mL.

Plates were incubated at 37° C. (5% CO₂) for a total of 12 days. Media was refreshed every 2-3 days with IL-15 (10 ng/mL final concentration) and IL-2 (R&D systems, 10 IU/mL final concentration). On day 11 lyophilized peptides (15-mer overlapping peptides) were solubilized in 100% DMSO and pooled by neoantigen to between 5 mg/mL and 20 mg/mL. Neoantigen peptides pools were added to cells for a final concentration of 2.5 μg/mL to 10 μg/mL. CEF Peptide Pool “Plus” (Cellular Technologies, Ltd.) was utilized as a positive control and DMSO at the same final concentration as the experimental peptides was utilized as a negative control. Protein Inhibitor Cocktail (eBioscience) was added to every well and plate was incubated overnight at 37° C. (5% CO₂).

On day 12, cells were stained for intracellular flow cytometry analysis. The cells were washed with PBS and stained with Live/Dead Fixable Aqua Dead Cell stain (Thermo-Fisher). Following the live/dead stain, cells were blocked using Biotin-Free Fc Receptor Blocker (Accurate Chemical & Scientific Corp). Extracellular cellular flow panel (1 μL/antibody per well in 50 μL) consisted of CD3 PerCP-Cy5.5 (Biolegend), CD4 BV421 (Biolegend), and CD8 APC-Cy7 (Biolegend). After extracellular staining, cells were fixed using Foxp3/Transcription Factor Staining Buffer Set (eBioscience) and stained for intracellular proteins (1:50 dilution) using TNFα FITC (R&D Systems) and IFNγ BV785 (Biolegend). Cells were washed and resuspended in stain buffer and analyzed using a BD Celesta flow cytometer.

Flow cytometry cell staining analysis was completed using FlowJo v10. Cells were gated on live, singlet, CD3+ cells. The CD8+ and CD4+ T cells were analyzed for TNFα/IFNγ expression and the frequency of double positive TNFα/IFNγ CD8+ and the frequency of double positive TNFα/IFNγ CD4+ T cells were recorded. Responses were assessed to be positive when the frequency of double positive TNFα/IFNγ CD8+ or TNFα/IFNγ CD4+ T cells due to stimulation with an experimental peptide pool was increased greater than or equal to 3-fold over the DMSO only negative control for that donor and at least 0.01%.

### In Vitro Binding of Neoantigens to Class I MHC

The 9 mer peptides identified by bioinformatics analysis were analyzed for their binding propensities to 6 common HLA class I alleles (HLA-A*01:01, A*02:01, A*03:01, A*24:02, B*07:02, B*08:01). The principle of the method is briefly described below and consists of two parts, one involving exchange of peptide with a positive control induced by Ultraviolet (UV) radiation, and the second is an enzyme immunoassay to detect stable HLA-peptide and empty HLA complexes.

HLA-bound peptides are critical for the stability of the HLA complex. A conditional HLA class I complex was stabilized by an UV-labile peptide utilizing a different peptide (Pos) for each HLA (Pos: HLA-A*01:01: CTELKLSDY(SEQ ID NO: 409), HLA-A*02:01: NLVPMVATV (SEQ ID NO: 410), HLA-A*03:01: LIYRRRLMK (SEQ ID NO: 411), HLA-A*24:02: LYSACFWWL (SEQ ID NO: 412), HLA-B*07:02: NPKASLLSL (SEQ ID NO: 413), HLA-B*08:01: ELRSRYWAI (SEQ ID NO: 414), which could be cleaved by UV irradiation when bound to the HLA molecule. Upon cleavage, the resulting peptide fragments dissociated from the HLA class I complex since their length was insufficient to bind stably to HLA. Under the conditions in which peptide cleavage was performed (neutral pH, on melting ice), the peptide-free HLA complex remained stable. Thus, when cleavage was performed in the presence of another HLA class I peptide of choice, this reaction resulted in net exchange of the cleaved UV-labile peptide Pos with the chosen peptide (Rodenko, B et al. (2006) Nature Protocols 1: 1120-32, Toebes, M et al. (2006) Nat Med 12: 246-51, Bakker, A H et al. (2008) Proc Natl Acad Sci USA 105: 3825-30).

The exchange efficiency between the peptide of interest and Pos was analyzed using an HLA class I ELISA. The combined technologies allowed the identification of ligands for an HLA molecule of interest which are potentially immunogenic.

Exchange control peptide Pos was a high affinity binder to the relevant HLA class I allele while exchange control peptide Neg was a non-binder. The UV control represented UV-irradiation of conditional HLA class I complex in the absence of a rescue peptide. The binding of exchange control peptide Neg (HLA-A*01:01: NPKASLLSL (SEQ ID NO: 413), HLA-A*02-01: IVTDFSVIK (SEQ ID NO: 416), HLA-A*03:01: NPKASLLSL (SEQ ID NO: 413), HLA-A*24:02: NLVPMVATV (SEQ ID NO: 410), HLA-B*07:02: LIYRRRLMK (SEQ ID NO: 411), HLA-B*08:01: NLVPMVATV (SEQ ID NO: 410) and all experimental peptides were evaluated relative to that of exchange control peptide Pos. The absorption of the latter peptide was set at 100%. This procedure resulted in a range of different exchange percentages that reflected the affinities of the different experimental peptides for the HLA allele used.

The HLA class I ELISA is an enzyme immunoassay based on the detection of beta2-microglobulin (B2M) of (peptide-stabilized) HLA class I complexes. To this end streptavidin was bound onto polystyrene microtiter wells. After washing and blocking, HLA complex present in exchange reaction mixtures or ELISA controls was captured by the streptavidin on the microtiter plate via its biotinylated heavy chain. Non-bound material was removed by washing. Subsequently, horseradish peroxidase (HRP)-conjugated antibody to human B2M was added. The HRP-conjugated antibody binds only to an intact HLA complex present in the microtiter well because unsuccessful peptide exchange results in disintegration of the original UV-sensitive HLA complex upon UV illumination. In the latter case B2M was removed during the washing step. After removal of non-bound HRP conjugate by washing, a substrate solution was added to the wells. A coloured product formed in proportion to the amount of intact HLA complex present in the samples. After the reaction was terminated by the addition of a stop solution, absorbance was measured in a microtiter plate reader. The absorbance was normalized to the absorbance of an exchange control peptide (represents 100%). Suboptimal HLA binding of peptides with a moderate to low affinity for HLA class I molecules can also be detected by this ELISA technique (Rodenko, B et al. (2006) Nature Protocols 1: 1120-32).

Peptides that had 10% or greater exchange efficiency in one of the 6 HLA alleles were considered for further immunogenicity testing and analysis.

### Example 2. Identification of Neoantigens by Bioinformatics

A computational framework was developed to analyze various prostate cancer RNA-seq datasets by bioinformatics means to identify common prostate cancer neoantigens resulting from aberrant transcriptional programs such as gene fusion events, intron retention, alternatively spliced variants and aberrant expression of developmentally silenced genes.

The datasets queried were:
The Genotype-Tissue Expression (GTEx) Consortium. This dataset encompasses 6137 RNA-seq datasets from 49 normal tissues and was used to annotate RNA features in normal tissues and assess frequency of potential prostate neoantigen candidates in normal tissue.
The Cancer Genome Atlas Prostate Adenocarcinoma (TCGA PRAD) (Cancer Genome Atlas Research Network. Cell. 2015 Nov. 5; 163(4):1011-25. doi: 10.1016/j.cell.2015.10.025). This dataset encompasses RNA-seq datasets from 508 prostate cancer subjects and was used to identify neoantigen candidates in localized prostate adenocarcinoma.
Stand Up To Cancer (SU2C) (Robinson D et al., Cell. 2015 May 21; 161(5):1215-1228. doi: 10.1016/j.cell.2015.05.001). This dataset encompasses RNA-seq datasets from 43 mCRPC subjects.

Quality control (QC) of raw data was conducted prior to analysis. Sequencing reads were first trimmed to remove Illumina's adapter sequences and reads mapping to human tRNA and rRNA were removed from downstream analysis. Reads were also trimmed of bases with poor base quality score (<10, PHRED scale; indicating a base with a 1 in 10 probability of being incorrect) at either ends. PHRED quality score measures the quality of the identification of the bases generated by automated DNA sequencing instruments. Trimmed reads with less than 25 bps were removed from the datasets. Additionally, following QC steps were considered to remove poor quality reads: remove reads having maximal base quality PHRED score of <15, remove reads with average base quality PHRED score of <10, remove reads having polyATCG rate >80%, remove RNA sequences in which one of the two reads failed.

Reads that passed the QC criteria were mapped to Human Genome Build 38 using ArrayStudio ((www_omicsoft_com/array-studio/) platform. Refseq gene model (release date Jun. 6, 2017) was used for annotation of novel RNA features.

The results published here are in whole or part based upon data generated by The Cancer Genome Atlas managed by the NCI and NHGRJ. Information about TCGA can be found at http://_cancergenme_nih_gov.

### Identification of Gene Fusion Events

FusionMap algorithm (Ge H et al., Bioinformatics. 2011 Jul. 15; 27(14):1922-8. doi: 10.1093/bioinformatics/btr310. Epub 2011 May 18) was used to identify gene fusion events in the prostate cancer datasets described above. FusionMap detects fusion junctions based on seed reads which contain the fusion breakpoint position in the middle region of the reads. The algorithm dynamically creates a pseudo fusion transcript/sequence library based on the consensus of mapped fusion junctions from the seed reads. FusionMap then aligns unmapped possible fusion reads to the pseudo fusion reference to further identify rescued reads. The program reports a list of detected fusion junctions, statistics of supporting reads, fusion gene pairs, as well as genomic locations of breakpoints and junction sequences, which characterize fusion genes comprehensively at base-pair resolution.

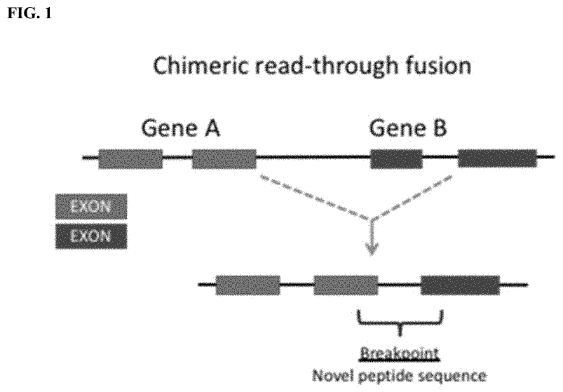

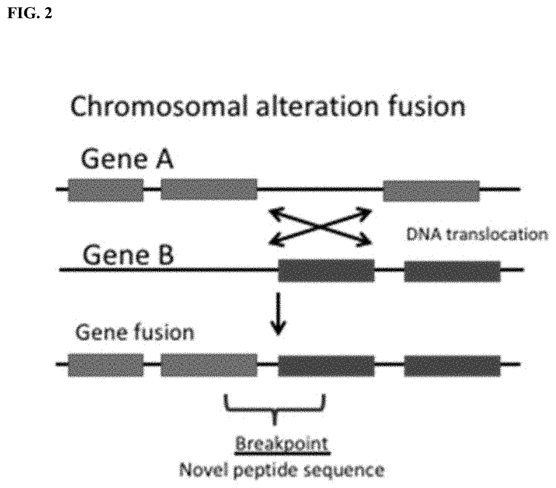

Neoantigens originating from chimeric read-through fusions as shown in FIG. 1 and fusions resulting from chromosomal alterations as shown in FIG. 2 were identified using FusionMap. Neoantigens were classified as originating from gene fusion events when following criteria were met: fusion junction was supported by at least two seed reads with different mapping positions in the genome, at least 4 sequencing reads (seed and rescued reads) parsing the junction, and at least one junction spanning read. The prevalence of neoantigens were queried in tumor tissue and normal tissue using the datasets mentioned above. Neoantigens were identified as common when the prevalence was identified to be >10% in at least one disease cohort (TCGA and SU2C) and <2% in normal tissue (6137 RNA-seq datasets from 49 normal tissues). Gene fusion events with less than 10% prevalence in disease cohort were included if they generated long stretches of novel peptide sequences or were present in genes of interest.

### Identification of Splice Variants

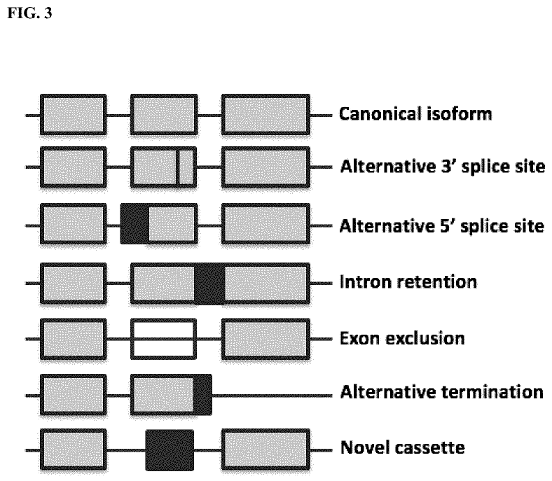

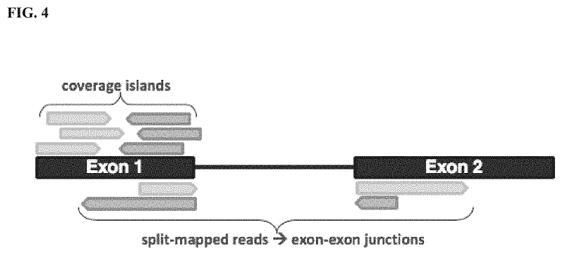

A custom bioinformatic software was developed to analyze paired-end RNA-seq data to identify potential neoantigens arising from alternative splicing events. Utilizing the developed process, splice variants with alternative 5′ or 3′ splice sites, retained introns, excluded exons, alternative terminations or insertion(s) of novel cassettes as show in in FIG. 3 were identified. The process identified splice variants that were not present in the RefSeq gene model through two main functionalities: 1) Identification of novel junctions based on reads with gaps of 6 or more bp and sequences of at least 15 bp aligned on each side of the gap, henceforth referred to as split-mapped reads. Novel junctions were reported if they were represented by at least 5 split-mapped reads and one mate pair of reads flanking the junction on each end. 2) Identification of islands of aligned reads, henceforth referred to as coverage islands. Further details on parameters used for determining island boundaries are described below. FIG. 4 shows the cartoon of the approach.

In order to differentiate reads mapping to intronic regions due to true splicing variation as opposed to genomic DNA and/or pre-mRNA contamination, two parameters were developed to establish the distribution of contamination across 200 highly expressed housekeeping genes. The tail ends of these distributions were then used as cut-offs for discovery of novel splice variants where relevant.
1. Intron depth of coverage (IDC): 90^{th} percentile depth of coverage for all housekeeping intronic bases. If the coverage of a particular region fell below this value, the first base where this occurred was defined as a coverage island boundary.
2. Intron/exon coverage ratio (IECR): 90^{th} percentile of the ratio between median intron coverage and median coverage of the nearest upstream exon of all housekeeping gene introns.

All reported splice variants were required to meet the following criteria:
Alternative 3′/5′ splice site identification:
Novel splice site was supported by at least 5 split-mapped reads and one mate pair of reads flanking the junction
Intronic region resulting from using the splice site (if applicable) exceeded IECR and entire region exceeded IDC
Novel cassette identification:
Two novel splice sites in an intronic region were supported by at least 5 split-mapped reads and one mate pair of reads flanking the junction
Region between the two splice sites exceeded IECR and entire region exceeded IDC
Intron retention identification:
Intronic region exceeded IECR and entire region exceeded IDC
At least 5 reads span both intron-exon boundaries, with at least 15 bp aligned on each side of the boundaries
Alternative termination identification:
3′ boundary defined as the edge of a coverage island that did not fall within 60 bp of the 3′ end of a canonical exon
Any intronic regions between 5′ end of a canonical exon and the 3′ boundary exceeded IECR and entire region exceeded IDC
Exon exclusion identification:
Novel junction was supported by at least 5 split-mapped reads and one mate pair of reads flanking the junction where one or more canonical exons were skipped

Neoantigens derived from aberrant splicing events were identified as common when the incidence was identified to be about >10% in disease cohort (TCGA and SU2C datasets) and about <1% in normal tissue (GTEx Consortium dataset).

### Identification of DNA Mutations (Point and Frameshift) Based Neoantigens

The TCGA, SU2C and the integrated DFCI/Sloane Kettering datasets (Integrated DFCI/Sloane Kettering dataset (Armenia et al., Nat Genet. 2018 May; 50(5):645-651. doi: 10.1038/s41588-018-0078-z. Epub 2018 Apr. 2) as described above containing exome sequencing data from patients with prostate cancer were examined. Mutation calls published by the consortia that generated these datasets were downloaded, and gene mutations that were present in >10% of the patient population or in genes known to be drivers of prostate cancer (such as AR) were identified. For each single point mutation chosen, a 17 mer peptide with the mutated amino acid at its center was identified for further validation studies.

### Splicing Isoform Prediction

In certain cases, there were multiple reading frames and exons upstream of the identified splicing events that could impact the canonical peptide sequence preceding the neoepitope sequence. In these genes, it was determined which canonical exons neighbored each neoepitope feature based on the split-mapped reads present at the exon boundaries. The most highly expressed isoform with the highest average expression in the disease cohort with the highest prevalence of the event that could contain the predicted neoepitope was chosen for translation into the corresponding protein by choice of the open reading frame associated with the isoform. The neoepitope portion of the protein sequence was extracted, with an additional 8 amino acid residues upstream of the first altered amino acid included and used for subsequent validation studies. A similar procedure was followed to identify putative immunogenic antigens from DNA frameshift alterations. For both frameshift deletions and insertions, the resulting DNA sequence was translated into the corresponding protein by choice of the appropriate open reading frame, and the frameshift altered portion of the protein sequence was extracted, with an additional 8 amino acid residues upstream of the first altered amino acid included.

Table 1 shows the gene origin, the specific mutation, the amino acid sequences of identified neoantigens with single amino acid mutations (M) and frequency in patients. Each mutation is bolded in Table 1. Table 2 shows their corresponding polynucleotide sequences. The mutant sequences are capitalized in Table 2. Patient frequency (%) in Table 1 was obtained from Armenia et al., Nat Genet 50(5): 645-651, 2018.

TABLE 1

| | | | Patient | | |
|---|---|---|---|---|---|
| Neoepitope | | | Frequency | | SEQ ID |
| ID | Gene | Mutation | (%) | Amino acid sequence | NO: |
| M1 | TP53 | R248Q | 1.0859 | SSCMGGMNQRPILTIIT | 1 |
| M2 | TP53 | R248W | 0.0987 | SSCMGGMNWRPILTIIT | 3 |
| M3 | TP53 | R273C | 0.6910 | LGRNSFEVCVCACPGRD | 5 |
| M4 | TP53 | R273L | 0.3949 | LGRNSFEVLVCACPGRD | 7 |
| M5 | TP53 | G2455 | 0.6910 | MCNSSCMGSMNRRPILT | 9 |
| M6 | TP53 | Y220C | 0.4936 | FRHSVVVPCEPPEVGSD | 11 |
| M7 | TP53 | R282W | 0.4936 | VCACPGRDWRTEEENLR | 13 |
| M8 | SPOP | F133C | 0.5923 | FVQGKDWGCKKFIRRDF | 15 |
| M9 | SPOP | F133I | 0.3949 | FVQGKDWGIKKFIRRDF | 17 |
| M10 | SPOP | F133L | 1.1846 | FVQGKDWGLKKFIRRDF | 19 |
| M11 | SPOP | F133S | 0.3949 | FVQGKDWGSKKFIRRDF | 21 |
| M12 | SPOP | F133V | 0.9872 | FVQGKDWGVKKFIRRDF | 23 |
| M13 | SPOP | W131C | 0.0987 | YRFVQGKDCGFKKFIRR | 25 |
| M14 | SPOP | W131G | 1.2833 | YRFVQGKDGGFKKFIRR | 27 |
| M15 | SPOP | W131L | 0.1974 | YRFVQGKDLGFKKFIRR | 29 |
| M16 | SPOP | W131R | 0.1974 | YRFVQGKDRGFKKFIRR | 31 |
| M17 | SPOP | W1315 | 0.0987 | YRFVQGKDSGFKKFIRR | 33 |
| M18 | KMT2D | R5214H | 0.1974 | YPVGYEATHIYWSLRTN | 35 |
| M19 | FOXA1 | R261C | 0.1974 | MFENGCYLCRQKRFKCE | 37 |
| M20 | FOXA1 | H247Q | 0.1974 | GKGSYWTLQPDSGNMFE | 39 |
| M21 | FOXA1 | H247L | 0.0987 | GKGSYWTLLPDSGNMFE | 41 |
| M22 | FOXA1 | H247N | 0.0987 | GKGSYWTLNPDSGNMFE | 43 |
| M23 | FOXA1 | H247Y | 0.0987 | GKGSYWTLYPDSGNMFE | 45 |
| M24 | FOXA1 | F266C | 0.0987 | CYLRRQKRCKCEKQPGA | 47 |
| M25 | FOXA1 | F266S | 0.0987 | CYLRRQKRSKCEKQPGA | 49 |
| M26 | FOXA1 | D226G | 0.0987 | IRHSLSFNGCFVKVARS | 51 |
| M27 | FOXA1 | D226N | 0.1974 | IRHSLSFNNCFVKVARS | 53 |
| M28 | FOXA1 | R219C | 0.0987 | QQRWQNSICHSLSFNDC | 55 |
| M29 | FOXA1 | R219S | 0.1974 | QQRWQNSISHSLSFNDC | 57 |
| M30 | FOXA1 | M253K | 0.1974 | TLHPDSGNKFENGCYLR | 59 |
| M31 | FOXA1 | M253R | 0.0987 | TLHPDSGNRFENGCYLR | 61 |
| M32 | CDK12 | R858W | 0.1974 | CHKKNFLHWDIKCSNIL | 63 |
| M33 | PTEN | R130Q | 0.2962 | IHCKAGKGQTGVMICAY | 65 |
| M34 | PTEN | V119F | 0.1974 | WLSEDDNHFAAIHCKAG | 67 |
| M35 | ATM | N2875S | 0.0987 | GLGDRHVQSILINEQSA | 69 |
| M36 | ATM | N2875K | 0.0987 | GLGDRHVQKILINEQSA | 71 |
| M37 | KDM6A | C1164S | 0.0987 | NINIGPGDSEWFVVPEG | 73 |
| M38 | KDM6A | C1164Y | 0.0987 | NINIGPGDYEWFVVPEG | 75 |
| M39 | PIK3CA | H1047R | 0.4936 | FMKQMNDARHGGWTTKM | 77 |
| M40 | PIK3CA | E545K | 0.2962 | RDPLSEITKQEKDFLWS | 79 |
| M41 | PIK3CA | E545G | 0.0987 | RDPLSEITGQEKDFLWS | 81 |
| M42 | PIK3CA | E545A | 0.0987 | RDPLSEITAQEKDFLWS | 83 |
| M43 | CTNNB1 | T41A | 0.4936 | SGIHSGATATAPSLSGK | 85 |
| M44 | CTNNB1 | D32A | 0.0987 | HWQQQSYLASGIHSGAT | 87 |
| M45 | CTNNB1 | D32H | 0.0987 | HWQQQSYLHSGIHSGAT | 89 |
| M46 | CTNNB1 | D32V | 0.0987 | HWQQQSYLVSGIHSGAT | 91 |
| M47 | CTNNB1 | D32Y | 0.1974 | HWQQQSYLYSGIHSGAT | 93 |
| M48 | CTNNB1 | S37A | 0.0987 | SYLDSGIHAGATTTAPS | 95 |
| M49 | CTNNB1 | S37C | 0.0987 | SYLDSGIHCGATTTAPS | 97 |
| M50 | CTNNB1 | S37F | 0.0987 | SYLDSGIHFGATTTAPS | 99 |
| M51 | CTNNB1 | S37Y | 0.0987 | SYLDSGIHYGATTTAPS | 101 |
| M52 | CTNNB1 | S45C | 0.0987 | SGATTTAPCLSGKGNPE | 103 |
| M53 | CTNNB1 | S45F | 0.0987 | SGATTTAPFLSGKGNPE | 105 |
| M54 | CTNNB1 | S45P | 0.0987 | SGATTTAPPLSGKGNPE | 107 |
| M55 | COL5A1 | T348K | 0.1974 | YVPSEDYYKPSPYDDLT | 109 |
| M56 | TAF1L | A869T | 0.1974 | IRKRLKLCTDFKRTGMD | 111 |
| M57 | MED12 | L1224F | 0.7897 | VDGAVFAVFKAVFVLGD | 113 |
| M58 | MED12 | V1223G | 0.0987 | IVDGAVFAGLKAVFVLG | 115 |
| M59 | MED12 | V1223L | 0.0987 | IVDGAVFALLKAVFVLG | 117 |
| M60 | MGA | R2435W | 0.1974 | THTANERRWRGEMRDLF | 119 |
| M61 | ARID1A | P1756R | 0.1974 | GRFSKVSSRAPMEGGEE | 121 |
| M62 | CUL3 | M299R | 0.4936 | GKTEDLGCRYKLFSRVP | 123 |
| M63 | USP7 | Q4H | 0.4936 | MNHHQQQQQQKA | 125 |
| M64 | SF3B1 | K700E | 0.1974 | HGLVDEQQEVRTISALA | 127 |
| M65 | U2AF1 | S34F | 0.2962 | VCRHGDRCFRLHNKPTF | 129 |
| M66 | CDC27 | Y73C | 0.1974 | SCTTPQCKCLLAKCCVD | 131 |
| M67 | CDC27 | N260H | 0.1974 | SILSKQVQHKPKTGRSL | 133 |
| M68 | BRAF | G469A | 0.2962 | QRIGSGSFATVYKGKWH | 135 |
| M69 | BRAF | K601E | 0.1974 | GDFGLATVESRWSGSHQ | 137 |
| M70 | RAG1 | R112C | 0.0987 | QANLRHLCCICGNSFRA | 139 |
| M71 | RAG1 | R112H | 0.1974 | QANLRHLCHICGNSFRA | 141 |
| M72 | CNOT3 | E20K | 0.3949 | DRCLKKVSKGVEQFEDI | 143 |
| M73 | CNOT3 | E70K | 0.2962 | IKTWVASNKIKDKRQLI | 145 |
| M74 | PIK3CB | E1051K | 0.2962 | QKFDEALRKSWTTKVNW | 147 |
| M75 | IDH1 | R132C | 0.1974 | WVKPIIIGCHAYGDQYR | 149 |
| M76 | IDH1 | R132G | 0.0987 | WVKPIIIGGHAYGDQYR | 151 |
| M77 | IDH1 | R132H | 0.4936 | WVKPIIIGHHAYGDQYR | 153 |
| M78 | KRAS | G12D | 0.1974 | YKLVVVGADGVGKSALT | 155 |
| M79 | KRAS | G12R | 0.2962 | YKLVVVGARGVGKSALT | 157 |
| M80 | KRAS | Q61K | 0.2962 | LDILDTAGKEEYSAMRD | 159 |
| M81 | KRAS | Q61L | 0.0987 | LDILDTAGLEEYSAMRD | 161 |
| M82 | KRAS | Q61R | 0.0987 | LDILDTAGREEYSAMRD | 163 |
| M83 | AKT1 | E17K | 0.4936 | EGWLHKRGKYIKTWRPR | 165 |
| M84 | AR | T878A | 1.2833 | IARELHQFAFDLLIKSH | 167 |
| M85 | AR | T878G | 0.0987 | IARELHQFGFDLLIKSH | 169 |
| M86 | AR | L702H | 1.0859 | QPDSFAALHSSLNELGE | 171 |
| M87 | AR | W742L | 0.1974 | QMAVIQYSLMGLMVFAM | 173 |
| M88 | AR | W742F | 0.0987 | QMAVIQYSFMGLMVFAM | 175 |

TABLE 2

| Neo- | | SEQ |
|---|---|---|
| epitope | | ID |
| ID | Polynucleotide sequence | NO: |
| M1 | agttcctgcatgggcggcatgaaccAgaggcccatcctcaccatcatcaca | 2 |
| M2 | agttcctgcatgggcggcatgaaTTggaggcccatcctcaccatcatcaca | 4 |
| M3 | ctgggacggaacagctttgaggtgTgtgtttgtgcctgtcctgggagagac | 6 |
| M4 | ctgggacggaacagctttgaggtgcTtgtttgtgcctgtcctgggagagac | 8 |
| M5 | atgtgtaacagttcctgcatgggcAgcatgaaccggaggcccatcctcacc | 10 |
| M6 | tttcgacatagtgtggtggtgccctGtgagccgcctgaggttggctctgac | 12 |
| M7 | gtttgtgcctgtcctgggagagaTTggcgcacagaggaagagaatctccgc | 14 |
| M8 | tttgtgcaaggcaaagactggggatGcaagaaattcatccgtagagatttt | 16 |
| M9 | tttgtgcaaggcaaagactggggaAtcaagaaattcatccgtagagatttt | 18 |
| M10 | tttgtgcaaggcaaagactggggattAaagaaattcatccgtagagatttt | 20 |
| M11 | tttgtgcaaggcaaagactggggatCcaagaaattcatccgtagagatttt | 22 |
| M12 | tttgtgcaaggcaaagactggggaGtcaagaaattcatccgtagagatttt | 24 |
| M13 | tataggtttgtgcaaggcaaagactgTggattcaagaaattcatccgtaga | 26 |
| M14 | tataggtttgtgcaaggcaaagacGggggattcaagaaattcatccgtaga | 28 |
| M15 | tataggtttgtgcaaggcaaagactggggattcaagaaattcatccgtaga | 30 |
| M16 | tataggtttgtgcaaggcaaagacCggggattcaagaaattcatccgtaga | 32 |
| M17 | tataggtttgtgcaaggcaaagactCgggattcaagaaattcatccgtaga | 34 |
| M18 | tatcccgtgggctacgaggccacgcAcatctattggagcctccgcaccaac | 36 |
| M19 | atgttcgagaacggctgctacttgTgccgccagaagcgcttcaagtgcgag | 38 |
| M20 | ggcaagggctcctactggacgctgcaGccggactccggcaacatgttcgag | 40 |
| M21 | ggcaagggctcctactggacgctgcTcccggactccggcaacatgttcgag | 42 |
| M22 | ggcaagggctcctactggacgctgAacccggactccggcaacatgttcgag | 44 |
| M23 | ggcaagggctcctactggacgctgTacccggactccggcaacatgttcgag | 46 |
| M24 | tgctacttgcgccgccagaagcgctGcaagtgcgagaagcagccgggggcc | 48 |
| M25 | tgctacttgcgccgccagaagcgctCcaagtgcgagaagcagccgggggcc | 50 |
| M26 | atccgccactcgctgtccttcaatgGctgcttcgtcaaggtggcacgctcc | 52 |
| M27 | atccgccactcgctgtccttcaatAactgcttcgtcaaggtggcacgctcc | 54 |
| M28 | cagcagcgctggcagaactccatcTgccactcgctgtccttcaatgactgc | 56 |
| M29 | cagcagcgctggcagaactccatcAgccactcgctgtccttcaatgactgc | 58 |
| M30 | acgctgcacccggactccggcaacaAgttcgagaacggctgctacttgcgc | 60 |
| M31 | acgctgcacccggactccggcaacaGgttcgagaacggctgctacttgcgc | 62 |
| M32 | tgtcacaaaaagaatttcctgcatTgggatattaagtgttctaacattttg | 64 |
| M33 | attcactgtaaagctggaaagggacAaactggtgtaatgatatgtgcatat | 66 |
| M34 | tggctaagtgaagatgacaatcatTttgcagcaattcactgtaaagctgga | 68 |
| M35 | ggacttggtgatagacatgtacagaGtatcttgataaatgagcagtcagca | 70 |
| M36 | ggacttggtgatagacatgtacagaaAatcttgataaatgagcagtcagca | 72 |
| M37 | aacataaatattggcccaggtgactCtgaatggtttgttgttcctgaaggt | 74 |
| M38 | aacataaatattggcccaggtgactAtgaatggtttgttgttcctgaaggt | 76 |
| M39 | ttcatgaaacaaatgaatgatgcacGtcatggtggctggacaacaaaaatg | 78 |
| M40 | cgagatcctctctctgaaatcactAagcaggagaaagattttctatggagt | 80 |
| M41 | cgagatcctctctctgaaatcactgGgcaggagaaagattttctatggagt | 82 |
| M42 | cgagatcctctctctgaaatcactgCgcaggagaaagattttctatggagt | 84 |
| M43 | tctggaatccattctggtgccactGccacagctccttctctgagtggtaaa | 86 |
| M44 | cactggcagcaacagtcttacctggCctctggaatccattctggtgccact | 88 |
| M45 | cactggcagcaacagtcttacctgCactctggaatccattctggtgccact | 90 |
| M46 | cactggcagcaacagtcttacctggTctctggaatccattctggtgccact | 92 |
| M47 | cactggcagcaacagtcttacctgTactctggaatccattctggtgccact | 94 |
| M48 | tcttacctggactctggaatccatGctggtgccactaccacagctccttct | 96 |
| M49 | tcttacctggactctggaatccattGtggtgccactaccacagctccttct | 98 |
| M50 | tcttacctggactctggaatccattTtggtgccactaccacagctccttct | 100 |
| M51 | tcttacctggactctggaatccattAtggtgccactaccacagctccttct | 102 |
| M52 | tctggtgccactaccacagctccttGtctgagtggtaaaggcaatcctgag | 104 |
| M53 | tctggtgccactaccacagctccttTtctgagtggtaaaggcaatcctgag | 106 |
| M54 | tctggtgccactaccacagctcctCctctgagtggtaaaggcaatcctgag | 108 |
| M55 | tacgtgcccagtgaggactactacaAgccctcaccgtatgatgacctcacc | 110 |
| M56 | atccggaagaggctaaagctctgcActgacttcaaacgcacagggatggat | 112 |
| M57 | gtggatggagccgtgtttgctgttTtcaaggctgtgtttgtacttggggat | 114 |
| M58 | atcgtggatggagccgtgtttgctgGtctcaaggctgtgtttgtacttggg | 116 |
| M59 | atcgtggatggagccgtgtttgctCttctcaaggctgtgtttgtacttggg | 118 |
| M60 | acacacactgccaatgagcggcggTggcgtggtgaaatgagggatctcttt | 120 |
| M61 | gggaggttcagcaaggtgtctagtcGagctcccatggagggtggggaagaa | 122 |
| M62 | ggaaagacagaagaccttggttgcaGgtacaagttatttagtcgtgtgcca | 124 |
| M63 | atgaaccaccaCcagcagcagcagcagcagaaagcg | 126 |
| M64 | catggtcttgtggatgagcagcagGaagttcggaccatcagtgctttggcc | 128 |
| M65 | gcatgtcgtcatggagacaggtgctTtcggttgcacaataaaccgacgttt | 130 |
| M66 | agttgtactacaccgcaatgcaaatGcctgcttgcaaaatgttgtgttgat | 132 |
| M67 | tccatattatctaaacaggttcaaCataaaccaaaaactggtcgaagttta | 134 |
| M68 | caaagaattggatctggatcatttgCaacagtctacaagggaaagtggcat | 136 |
| M69 | ggtgattttggtctagctacagtgGaatctcgatggagtgggtcccatcag | 138 |
| M70 | caagccaaccttcgacatctctgcTgcatctgtgggaattcttttagagct | 140 |
| M71 | caagccaaccttcgacatctctgccAcatctgtgggaattcttttagagct | 142 |
| M72 | gatcgctgcctcaagaaggtgtccAagggcgtggagcagtttgaagatatt | 144 |
| M73 | atcaagacatgggtagcgtccaacAagatcaaggacaagaggcagcttata | 146 |
| M74 | caaaaatttgatgaggcgctcaggAaaagctggactactaaagtgaactgg | 148 |
| M75 | tgggtaaaacctatcatcataggtTgtcatgcttatggggatcaatacaga | 150 |
| M76 | tgggtaaaacctatcatcataggtGgtcatgcttatggggatcaatacaga | 152 |
| M77 | tgggtaaaacctatcatcataggtcAtcatgcttatggggatcaatacaga | 154 |
| M78 | tataagctggtggtggtgggcgccgAcggtgtgggcaagagtgcgctgacc | 156 |
| M79 | tataagctggtggtggtgggcgccCgcggtgtgggcaagagtgcgctgacc | 158 |
| M80 | ttggacatcctggataccgccggAAaggaggagtacagcgccatgcgggac | 160 |
| M81 | ttggacatcctggataccgccggccTggaggagtacagcgccatgcgggac | 162 |
| M82 | ttggacatcctggataccgccggccGggaggagtacagcgccatgcgggac | 164 |
| M83 | gagggttggctgcacaaacgagggAagtacatcaagacctggcggccacgc | 166 |
| M84 | attgcgagagagctgcatcagttcGcttttgacctgctaatcaagtcacac | 168 |
| M85 | attgcgagagagctgcatcagttcGGttttgacctgctaatcaagtcacac | 170 |
| M86 | cagcccgactcctttgcagccttgcActctagcctcaatgaactgggagag | 172 |
| M87 | cagatggctgtcattcagtactcctTgatggggctcatggtgtttgccatg | 174 |
| M88 | cagatggctgtcattcagtactcctTTatggggctcatggtgtttgccatg | 176 |

Table 3 shows the gene origin, the specific frameshift mutation (FR), the amino acid sequences of the identified neoantigens that arose from frameshift events and frequency of the mutation in patients. The wild-type sequence is bolded in Table 3, followed by the novel sequence due to frameshift. Table 4 shows their corresponding polynucleotide sequences. The mutant sequences are capitalized in Table 4. Patient frequency (%) in Table 3 was obtained from Armenia et al., Nat Genet 50(5): 645-651, 2018.

TABLE 3

| Neo- | | | Patient | | SEQ |
|---|---|---|---|---|---|
| epitope | | | Frequency | | ID |
| ID | Gene | Frameshift | (%) | Amino acid sequence | NO: |
| FR1 | ZFHX3 | E763Sfs*61 | 0.2962 | QNLQNGGGSRSSATLP | 177 |
| | | | | GRRRRRWLRRRRQPISV | |
| | | | | APAGPPRRPNQKPNPPG | |
| | | | | GARCVIMRPTWPGTSAFT | |
| FR2 | ZFHX3 | E763Gfs*26 | 0.0987 | QNLQNGGGGAGLQPH | 179 |
| | | | | CRGGGGGGGCGGGGSQYQ | |
| FR3 | APC | T1556Nfs*3 | 0.3949 | NQEKEAEKNY | 181 |
| FR4 | SPEN | A2105Lfs*33 | 0.1974 | DAAVSPRGLQHRQGRG | 183 |
| | | | | NLGWWQSPLRKVRVPK | |
| | | | | RRMVYHPS | |
| FR5 | BRCA2 | T3085Nfs*26 | 0.1974 | FVVSVVKKNRTCPFRL | 185 |
| | | | | FVRRMLQFTGNKVLDRP | |
| FR6 | BRCA2 | K2674Rfs*2 | 0.1974 | RSRRSAIKR | 187 |
| FR7 | ARID4A | S1067Rfs*16 | 0.2962 | SIIVQERERAERRVRRG | 189 |
| | | | | QVMEIVD | |
| FR8 | SMARCA | N770Kfs*28 | 0.1974 | NNLVTEKKHRNVQCH | 191 |
| | D1 | | | DAVEENGQSSFITSPILHS | |
| FR9 | RNF43 | G659Vfs*41 | 0.3949 | HPQRKRRGVPPSPPLA | 193 |
| | | | | LGPRMQLCTQLARFFPI | |
| | | | | TPPVWHILGPQRHTP | |
| FR10 | AXIN2 | G665Afs*24 | 0.1974 | ASRHHLWGATAGTPA | 195 |
| | | | | PPPVPTCSPRTLRCLP | |
| FR11 | ERF | L525Sfs*6 | 0.2962 | GPGEAGGPSPQGG | 197 |
| FR12 | ERF | G299Efs*12 | 0.2962 | GGGPSGSGEAPTSPSALRT | 199 |
| FR13 | CHD3 | R599Vfs*16 | 0.3949 | GNPDVPPPVLFKADQS | 201 |
| | | | | ESSLSSG | |
| FR14 | KMT2C | S143Vfs*3 | 0.2962 | AFCYCGEKVP | 203 |
| FR15 | FOXA1 | M253_N256 | 0.0987 | TLHPDSGNGCYLRRQK | 205 |
| | | del | | | |
| FR16 | FOXA1 | F254_N256d | 0.2962 | LHPDSGNMYGCYLRRQ | 207 |
| | | elinsY | | | |
| FR17 | FOXA1 | F254_G257d | 0.0987 | LHPDSGNMCCYLRRQKR | 209 |
| | | elinsC | | | |

TABLE 4

| Neoantigen | | SEQ |
|---|---|---|
| ID | Polynucleotide sequence | ID NO: |
| FR1 | Cagaacctgcagaatggaggggggagcaggtcttcagccacactgccggggcggcgg | 178 |
| | cggcggcggtggctgcggcggcggcggcagccaatatcagtagctcctgcggggccc | |
| | cctcgccgaccaaaccaaaaaccaaacccacctggcggtgcgaggtgtgtgattatgag | |
| | accaacgtggccaggaacctccgcattcaca | |
| FR2 | cagaacctgcagaatggaggggggGgagcaggtcttcagccacactgccggggcggc | 180 |
| | ggcggcggcggtggctgcggcggcggcggcagccaatatcagtag | |
| FR3 | aaccaagagaaagaggcagAaaaaaactattga | 182 |
| FR4 | gatgctgctgtcagtcccagggggctgcagcacaggcaggggagagggaatctggggt | 184 |
| | ggtggcagtctcccctgagaaaagtgagagtccccaaaaggaggatggtttatcatccca | |
| | gttga | |
| FR5 | tttgtcgtttctgttgtgaAaaaaaacaggacttgcccctttcgtctatttgtcagacgaat | 186 |
| | gttacaatttactggcaataaagttttggatagaccttaa | |
| FR6 | agaagcagaagatcggctataaaaagataatg | 188 |
| FR7 | agtataattgtacaAGagagagagagagcagagagaagggtcagaagaggccaagtg | 190 |
| | atggaaatagtggattaa | |
| FR8 | aataacttggtcacagAaaaaaaacacagaaatgtgcaatgtcatgatgcagttgaggaa | 192 |
| | aatggccaatcatcctttattacatcgccaatattacacagctgaaa | |
| FR9 | cacccacagaggaaaaggcggggggtccctccgagcccacccctggctctcggcccca | 194 |
| | ggatgcaactgtgcacccagcttgccagatttttccccattacacccccagtgtggcatatc | |
| | cttggtccccagaggcacaccccttgatc | |
| FR10 | gccagccggcaccatctgtggggggcaacagcgggcacccccgcaccaccccccgtg | 196 |
| | cccacctgttcacccaggaccctgcgatgcctcccctgacc | |
| FR11 | gggcctggggaggctgggggcccctcaccccaaggcgggtgagc | 198 |
| FR12 | ggcggggggcccagcggctcaggggaggctcccacttctccttcagccctgaggacat | 200 |
| | gaaa | |
| FR13 | ggaaatccagatgtcccaccccccgtcctcttcaaggcagatcagagcgagagttctttgt | 202 |
| | caagtgggtag | |
| FR14 | gctttttgttactgtggggaaaaagaccttagga | 204 |
| FR15 | acgctgcacccggactccggcaacggctgctacttgcgccgccagaagcg | 206 |
| FR16 | acgctgcacccggactccggcaacatgtacggctgctacttgcgccgccagaa | 208 |
| FR17 | ctgcacccggactccggcaacatgtgctgctacttgcgccgccagaagcgc | 210 |

Table 5 shows the gene origin and amino acid sequences of the identified neoantigens that arose from gene fusion (FUS) events. Table 6 shows their corresponding polynucleotide sequences. Table 7 shows the prevalence of the FUS neoantigens in analyzed databases.

TABLE 5

| Neoantigen | | | SEQ ID |
|---|---|---|---|
| ID | Fusion Gene | Amino acid sequence | NO: |
| FUS1 | SLC45A3-> | CGASACDVSLIAMDSA | 211 |
| | ELK4 | | |
| FUS2 | ARHGEF38-> | TEYNQKLQVNQFSESK | 213 |
| | ARHGEF38- | | |
| | IT1 | | |
| FUS3 | MSMB-> | TEISCCTLSSEENEYLPRPEWQLQ | 215 |
| | NCOA4 | | |
| FUS4 | LIPE->CNFN | GLVSFGEHFCLPCALC | 217 |
| FUS5 | TMPRSS2-> | NSKMALNSEALSVVSE | 219 |
| | ERG | | |
| FUS6 | TMPRSS2-> | CEERGAAGSLISCE | 221 |
| | ERG | | |
| FUS7 | NME4->DECR2 | LWFQSSELSPTGAPWPSRRPTWRGTTVSPRT | 223 |
| | | ATSSARTCCGTKWPSSQEAALGLGSGLLRFS | |
| | | CGTAAIR | |
| FUS8 | INCA1-> | WGMELAASRRFSWDHHSAGGPPRVPSVRS | 225 |
| | CAMTA2 | GAAQVQPKDPLPLRTLAGCLARTAHLRPGA | |
| | | ESLPQPQLHCT | |
| FUS9 | AP5S1->MAVS | KEQILAVASLVSSQSIHPSWGQSPLSRI | 227 |
| FUS10 | DIP2A-> | LELELSEGVCFRLR | 229 |
| | DIP2A-IT1 | | |
| FUS11 | MBTPS2->YY2 | QQLRIFCAAMASNEDFS | 231 |
| FUS15 | D2HGDH-> | HVVGYGHLDTSGSSSSSSWP | 345 |
| | GAL3ST2 | | |
| FUS18 | OPN3->CHML | DGFSGSLFAVVTRRCYFLKWRTIFPQSLMWL | 233 |
| FUS19 | GTF2F1->PSPN | KMHFSLKEHPPPPCPP | 235 |
| FUS23 | NUDT14-> | DLRRVATYCAPLPSSWRPGTGTTIPPRMRSC | 237 |
| | JAG2 | | |
| FUS24 | DMPK->SIX5 | LQERMELLACGAERGAGGWGGGGGGGGG | 239 |
| | | DRRGGGGSAPALADFAGGRG | |

TABLE 6

| Neo- | | SEQ |
|---|---|---|
| antigen | | ID |
| ID | Polynucleotide sequence | NO: |
| FUS1 | TGCGGGGCCTCTGCCTGTGATGTCTCCCTCATTGCTA | 212 |
| | TGGACAGTGCT | |
| FUS2 | ACCGAATACAACCAGAAATTACAAGTGAATCAATTT | 214 |
| | AGTGAATCCAAA | |
| FUS3 | ACAGAAATTTCATGTTGCACCCTGAGCAGTGAGGAG | 216 |
| | AATGAATACCTTCCAAGACCAGAGTGGCAGCTCCAG | |
| FUS4 | GGGCTGGTGTCCTTCGGGGAGCACTTTTGTCTGCCCT | 218 |
| | GCGCCCTCTGCCA | |
| FUS5 | AACAGCAAGATGGCTTTGAACTCAGAAGCCTTATCA | 220 |
| | GTTGTGAGTGAG | |
| FUS6 | TGTGAGGAGCGCGGCGCGGCAGGAAGCCTTATCAGT | 222 |
| | TGTGAG | |
| FUS7 | CTGTGGTTCCAGAGCAGTGAGCTGTCCCCGACGGGA | 224 |
| | GCGCCATGGCCCAGCCGCCGCCCGACGTGGAGGGGG | |
| | ACGACTGTCTCCCCGCGTACCGCCACCTCTTCTGCCC | |
| | GGACCTGCTGCGGGACAAAGTGGCCTTCATCACAGG | |
| | AGGCGGCTCTGGGATTGGGTTCCGGATTGCTGAGAT | |
| | TTTCATGCGGCACGGCTGCCATACGG | |
| FUS8 | TGGGGGATGGAGTTGGCAGCGTCTCGGAGGTTCTCC | 226 |
| | TGGGACCACCACTCCGCCGGGGGGCCGCCCAGAGTG | |
| | CCAAGCGTCCGATCCGGCGCCGCCCAAGTGCAGCCC | |
| | AAGGACCCGCTCCCGCTCCGCACCCTGGCAGGCTGC | |
| | CTAGCCAGGACTGCGCACCTGCGCCCTGGGGCGGAG | |
| | TCCTTACCCCAACCCCAGCTTCACTGCACA | |
| FUS9 | AAGGAACAGATTTTAGCTGTGGCCAGTCTCGTTTCCT | 228 |
| | CTCAGTCCATCCACCCTTCATGGGGCCAGAGCCCTCT | |
| | CTCCAGAATC | |
| FUS10 | CTGGAGCTGGAGCTGTCGGAAGGAGTCTGCTTCAGA | 230 |
| | TTAAGA | |
| FUS11 | CAGCAGCTAAGGATATTTTGTGCAGCCATGGCCTCC | 232 |
| | AACGAAGATTTCTCCA | |
| FUS15 | CACGTGGTGGGCTATGGCCACCTTGATACTTCCGGGT | 346 |
| | CATCCTCCTCCTCCTCCTGGCCC | |
| FUS18 | GACGGGTTTAGCGGCAGCCTCTTCGCAGTTGTCACC | 234 |
| | AGACGCTGTTACTTCCTAAAATGGCGGACAATCTTCC | |
| | CACAGAGTTTGATGTGGTTA | |
| FUS19 | AAAATGCACTTCTCCCTCAAGGAGCACCCACCGCCC | 236 |
| | CCTTGCCCGCCT | |
| FUS23 | GATCTGCGCCGGGTCGCCACATACTGCGCTCCTTTAC | 238 |
| | CCTCATCGTGGAGGCCTGGGACTGGGACAACGATAC | |
| | CACCCCGAATGAGGAGCTGC | |
| FUS24 | TTGCAGGAGCGGATGGAGTTGCTTGCCTGCGGAGCC | 240 |
| | GAGCGCGGGGCCGGCGGCTGGGGGGGAGGCGGTGG | |
| | CGGCGGCGGCGGCGACCGAAGAGGAGGAGGAGGAA | |
| | GCGCGCCAGCTCTTGCAGACTTTGCAGGCGGCCGAG | |
| | GG | |

TABLE 7

| | TCGA | SU2C |
|---|---|---|
| Neoantigen ID | (%) | (%) |
| FUS1 | 30.51 | 23.26 |
| FUS2 | 63.58 | 46.51 |
| FUS3 | 35.04 | 23.26 |
| FUS4 | 12.20 | 11.63 |
| FUS5 | 12.40 | 18.60 |
| FUS6 | 21.46 | 32.56 |
| FUS7 | 3.35 | 16.28 |
| FUS8 | 1.18 | 32.56 |
| FUS9 | N.O. | 18.60 |
| FUS10 | N.O. | 13.95 |
| FUS11 | 1.57 | 13.95 |
| FUS15 | 0.39 | 9.30 |
| FUS18 | 0.39 | 9.30 |
| FUS19 | 8.86 | 30.23 |
| FUS23 | N.O. | 9.30 |
| FUS24 | N.O. | 9.30 |

N.O. not observed

Table 8 shows the gene origin and amino acid sequences of the identified neoantigens that arose from alternative splicing (AS) events. Table 9 shows their corresponding polynucleotide sequences. Table 10 shows the prevalence of the AS neoantigens in analyzed databases.

TABLE 8

| Neo- | | | SEQ |
|---|---|---|---|
| epitope | | | ID |
| ID | Gene | Amino acid sequence | NO: |
| AS1 | ABCC4 | LTFLDFIQVTLRVMSGSQMENGSSYFFK | 241 |
| | | PFSWGLGVGLSAWLCVMLT | |
| AS2 | SLC30A4 | FMIGELVGELCCQLTFRLPFLESLCQAV | 243 |
| | | VTQALRFNPSFQEVCIYQDTDLM | |
| AS3 | DNAH8 | VAMMVPDRQVHYDFGL | 245 |
| AS4 | NCAPD3 | WCPLDLRLGSTGCLTCRHHQTSHE | 247 |
| AS5 | DHDH | VVGRRHETAPQPLLVPDRAGGEGGA | 249 |
| AS6 | ACSM1 | DYWAQKEKISIPRTHLC | 251 |
| AS7 | ACSM1 | DYWAQKEKGSSSFLRPSC | 253 |
| AS8 | CACNA1D | LVLGVLSGHSGSRL | 255 |
| AS9 | CACNA1D | PVPTATPGVRSVTSPQGLGLFLKFI | 257 |
| AS10 | CHRNA5 | KENDVREVCDVYLQMQIFFHFKFRSYF | 259 |
| | | H | |
| AS11, | CPNE7 | VPFRELKNQRTAQGAPGIHHAASPVAA | 261 |
| AS33 | | NLCDPARHAQHTRIPCGAGQVRAGRGP | |
| | | EAGGGVLQPQRPAPEKPGCPCRRGQPRL | |
| | | HTVKMWRA | |
| AS12 | EVPL | FARKMLEKVHRQHLQLSHNSQE | 263 |
| AS13 | GRIN3A | KRSFAVTERII | 265 |
| AS14 | IQCG | MFLRKEQQVGPHSFSML | 267 |
| AS15 | LRRC45 | VLRFLDLKVRYLHS | 269 |
| AS16 | LRRC45 | GNTTLQQLGEASQAPSGSLIPLRLPLLW | 271 |
| | | EVRG | |
| AS17 | MPHOSPH | GLNLNTDRPGGYSYSIWWKNNAKNR | 273 |
| | 9 | | |
| AS18 | NWD1 | WKFEMSYTVGGPPPHVHARPRHWKTD | 275 |
| | | R | |
| AS19 | NWD1 | QWQHYHRSGEAAGTPLWRPTRN | 277 |
| AS20 | PFKFB4 | KVLNEIDAVVTVPPSLSTSQIPQGCCII | 279 |
| | | L | |
| AS21 | RECQL4 | ANLKGTLQVRSGQAVSPR | 281 |
| AS22 | TONSL | LQAAASGQGKQGVPCPWGCCAYAESP | 283 |
| | | RALISGDAPSQVEREVPGPCLNTHSLSH | |
| | | RSPQLPGLPHPKQPSV | |
| AS23 | ZNF614 | KIQNKNCPD | 285 |
| AS32 | TONSL | GEVELSEGGEGQRHLAFPWACSGPGWR | 287 |
| | | GVCCAAVEPA | |
| AS63 | TDRD1 | IEMKKLLKS | 289 |
| AS34 | LRRC45 | KMRAIQAEGGHGQACCGGAWGWAPG | 291 |
| | | DGGPQGMLTHTLPTLGFQSAWTWRRED | |
| | | ADRAWRTPKACASRRWSI | |
| AS35 | AMACR | LLEPFRRGEPGPRGLLSGSSRGGEGPGR | 293 |
| | | SIEAAPATPLPCCRKNPCRPQPSRFLPP | |
| | | RVLLVIILPKLDCPKLGF | |
| AS36 | CCNF | PSGRRTKRLVTLRSGCAIQCWHPRAGP | 295 |
| | | VPSALPHTERPPRLVRGAADPRTVTLGR | |
| | | SPAVMPRAPA | |
| AS37 | RECQL4 | CHLFLQPQVGTPPPHTASARAPSGPPHP | 297 |
| | | HESCPAGRRPARAAQTCARRQHGLPGC | |
| | | EEAGTARVPSLHLHLHQAALGAGRGRG | |
| | | WGEACAQVPPSRG | |
| AS38 | LRRC45 | KELKLEQQVGGQGLRGVGQGVRGGFV | 299 |
| | | TLTTHTPFPSQEAAERESK | |
| AS39 | CCNF | GEISQEEVPPSRHLGVSWGAGVWAGLTL | 301 |
| | | GASAPPNSSFPSGAELQPVVCCIRSDTR | |
| | | QPRPPDFPQHRGDPRLPQLSLGAENQTV | |
| | | SYPAFWLRHTMLASSCRPSSLSASSHRE | |
| | | APKACQGSSRSQDSDPGTEPCSHASGPC | |
| | | VTSTVSSPGLLPQRLLPLALTGLPVEED | |
| | | GFEHAGA | |
| AS40 | LRRC45 | DCMLSEEGGQARRGGSLCSLAAHTIASA | 303 |
| | | ARGRFLSRLSNFCAVVKASRGAPSCTWE | |
| AS41 | RHPN1 | EAFQRAAGEGGPGRGGARRGARVLQSP | 305 |
| | | FCRAGAGEWLGHQSLR | |
| AS42 | SLC39A4 | PEPRRLSPGEPRGRPRKGWGIWGLCGA | 307 |
| | | RVGPKAWR | |
| AS43 | CPNE7 | VPFRELKNVSVLEGLRQGRLGGPCSCH | 309 |
| | | CPRPSQARLTPVDVAGPFLCLGDPGLFP | |
| | | PVKSSI | |
| AS44 | FASN | FVSLTAIQMASSATPWGRWPVATPTAA | 311 |
| | | CPRRRPSSLPTGGDSASKKPISRRAPWQ | |
| | | PWACPGRSVNSAAPRAWCPPATTPRTQS | |
| | | PSRDLRPRCLSSWSS | |
| AS45 | RBM47 | PVAIKPGTGPPNNSSIHGGSKRSENSYC | 313 |
| | | RDLRGQLRAICCSSYSHDRHTTEERGSR | |
| | | GRHVWRIRRLHTSGLPCCCHSGPHPRRL | |
| | | PDILRLVTSTKTDHTNTTEGTLDYL | |
| AS46 | SERINC5 | KWNKNWTATLGALTIRGHKLLCHLPHL | 315 |
| | | LSSVQQTCRSSSR | |
| AS47 | AGRN | FKKFDGPCGERGGGRTARALWARGDS | 317 |
| | | VLTPALDPQTPVRAPSLTRAAAAV | |
| AS48 | SYT17 | ENASLVFTGSNSPIPACELSSHPAHGIS | 319 |
| | | PWIPSPGNEHFHGIKKQVKAIKVE | |
| AS49 | PDF | RLTQRLVQGWTPMENRWCGRRAGGQPA | 321 |
| | | SSSTRWTTCRAACLLTKWTAGRSQTSIG | |
| AS50 | LRIF1 | ENSGNASRWLHVPSSSDDWLGWKKSSA | 323 |
| | | ITSNS | |
| AS51 | CPNE7 | GMECTLGQVGAPSPRREEDGWRGGHS | 325 |
| | | RFKADVPAPQGPCWGGQPGSAPSSAPPE | |
| | | QSLLD | |
| AS52 | ILDR1 | KGSVERRSVSLGHPAEGWAWAERSLQP | 327 |
| | | GMTTANTGCLSFHHRGCLLPVLPKLHCG | |
| | | LGGLPLVRAKEIKRVQRAGESSLPVKGL | |
| | | LTVASAVIAVLWGRPSEVTGENEAQHD | |
| AS53 | PEX10 | FGLTTLAGRSSHGTSGLRAATHTKSGD | 329 |
| | | GGQGAARQCEKLLELARATRPWGRSTS | |
| | | ASSRWTHRGYMCPPRCAVACW | |
| AS54 | ABCC4 | IIDSDKIMAVCMGCLLTRHVQCQAMEM | 331 |
| | | QQ | |
| AS55 | SPOCK1 | DGHSYTSKVNCLLLQDGFHGCVSITGA | 333 |
| | | AGRRNLSIFLFLMLCKLEFHAC | |
| AS56 | TM9SF3 | LLNAEDYRCAIHSKEIYLLSPSPHQAMD | 335 |
| | | KFSLCCINCNLCLHVFLLLLFFQNKDVW | |
| | | LISNIILLWIYGGI | |
| AS57 | KLK3 | TGGKSTCSAPGPQSLPSTPFSTYPQWVI | 337 |
| | | LITEL | |
| AS58 | CREB3L1 | VETLENANSFSSGIQPLLCSLIGLENPT | 339 |
| AS59 | ACSL3 | AGAGTISEGSVLHGQRLECDARRFFGCG | 341 |
| | | TTILAEWEHH | |
| AS55.1 | SPOCK1 | DGHSYTSKVNCLLLQDGFHGCVSITGA | 385 |
| | | AGRRNLSIFLFLMLCKLEFHA | |

TABLE 9

| Neo- | | SEQ |
|---|---|---|
| epitope | | ID |
| ID | Polynucleotide sequence | NO: |
| AS1 | CTGACGTTTTTAGATTTCATCCAGGTAACGTTGAGAGT | 242 |
| | AATGTCAGGATCTCAAATGGAAAACGGAAGTTCCTAT | |
| | TTTTTCAAGCCCTTTTCATGGGGTCTGGGGGTGGGACT | |
| | CTCGGCCTGGCTGTGTGTAATGTTAACT | |
| AS2 | TTCATGATTGGAGAACTTGTAGGTGAGTTGTGTTGCCA | 244 |
| | ACTCACTTTCCGTTTACCTTTCCTCGAGAGTCTTTGTC | |
| | AAGCTGTAGTTACACAGGCTTTGAGGTTTAACCCATCT | |
| | TTTCAGGAAGTTTGTATTTATCAAGACACTGATCTCAT | |
| | G | |
| AS3 | GTTGCTATGATGGTTCCTGATAGACAGGTTCATTATGA | 246 |
| | CTTTGGATTG | |
| AS4 | TGGTGTCCGCTGGATCTTAGACTCGGTTCCACTGGATG | 248 |
| | TCTCACATGCAGACATCATCAAACGTCACATGAG | |
| AS5 | GTCGTGGGAAGGCGTCATGAAACAGCTCCTCAACCCCT | 250 |
| | GCTGGTGCCCGACCGAGCTGGTGGTGAAGGGGGAGCA | |
| AS6 | GACTACTGGGCTCAAAAGGAGAAGATCAGCATCCCCA | 252 |
| | GAACACACCTGTGT | |
| AS7 | GACTACTGGGCTCAAAAGGAGAAGGGATCATCTTCAT | 254 |
| | TCCTGCGACCATCCTGT | |
| AS8 | CTTGTACTTGGTGTATTGAGCGGGCACAGTGGCTCACG | 256 |
| | CCTA | |
| AS9 | CCTGTCCCAACTGCTACACCTGGGGTAAGATCAGTGAC | 258 |
| | TAGTCCCCAGGGGCTGGGCCTTTTCCTTAAGTTTATT | |
| AS10 | AAGGAAAATGATGTCCGTGAGGTCTGTGATGTGTATTT | 260 |
| | ACAAATGCAGATCTTCTTCCATTTTAAGTTCAGAAGTT | |
| | ACTTTCAT | |
| AS11, | GTGCCCTTCCGGGAGCTCAAGAACCAGAGAACAGCAC | 262 |
| AS33 | AAGGGGCTCCTGGGATCCACCACGCGGCTTCCCCCGTT | |
| | GCTGCCAACCTCTGCGACCCGGCGAGACACGCACAGC | |
| | ACACACGCATCCCCTGCGGCGCTGGCCAAGTGCGTGC | |
| | TGGCCGAGGTCCCGAAGCAGGTGGTGGAGTACTACAG | |
| | CCACAGAGGCCTGCCCCCGAGAAGCCTGGGTGTCCCT | |
| | GCCGGAGAGGCCAGCCCAGGCTGCACACCGTGAAGAT | |
| | GTGGAGGGCG | |
| AS12 | TTTGCTAGAAAAATGCTGGAGAAGGTACACAGACAAC | 264 |
| | ACCTACAGCTTTCCCACAATAGCCAGGAA | |
| AS13 | AAGAGAAGTTTTGCTGTCACGGAGAGGATCATC | 266 |
| AS14 | ATGTTCCTTAGAAAGGAGCAGCAGGTGGGTCCCCACA | 268 |
| | GCTTTTCTATGCTT | |
| AS15 | GTGCTGCGCTTTCTGGACTTAAAGGTGAGATACCTGCA | 270 |
| | CTCT | |
| AS16 | GGCAACACCACCCTCCAGCAGCTGGGTGAGGCCTCCC | 272 |
| | AGGCGCCCTCAGGCTCCCTCATCCCTCTGAGGCTGCCT | |
| | CTGCTCTGGGAAGTGAGGGGC | |
| AS17 | GGACTGAACTTAAATACTGATAGACCAGGTGGTTACAG | 274 |
| | CTATTCAATTTGGTGGAAAAACAATGCCAAGAACAGA | |
| AS18 | TGGAAATTCGAGATGAGCTACACGGTGGGTGGCCCGC | 276 |
| | CTCCCCATGTTCATGCTAGACCCAGGCATTGGAAAACT | |
| | GATAGA | |
| AS19 | CAGTGGCAGCACTACCACCGGTCAGGTGAGGCCGCAG | 278 |
| | GGACTCCCCTCTGGAGACCCACAAGAAAC | |
| AS20 | AAGGTCCTCAACGAGATCGATGCGGTAGTTACCGTCC | 280 |
| | CTCCCTCCCTGTCTACCTCCCAGATACCGCAGGGCTGC | |
| | TGCATCATATTG | |
| AS21 | GCCAATCTGAAAGGCACCCTGCAGGTGAGGAGTGGGC | 282 |
| | AGGCAGTGAGTCCACGC | |
| AS22 | CTCCAGGCGGCTGCCTCGGGCCAAGGCAAGCAGGGCG | 284 |
| | TCCCTTGTCCCTGGGGTTGCTGTGCCTACGCTGAGAGT | |
| | CCCCGGGCCCTGATTTCGGGAGATGCTCCATCACAGGT | |
| | GGAGCGGGAGGTGCCGGGCCCCTGCCTCAACACGCAT | |
| | TCTCTCTCCCACAGATCCCCACAGCTCCCAGGCCTTCC | |
| | ACACCCCAAGCAGCCTTCTGTT | |
| AS23 | AAAATTCAGAATAAAAATTGTCCAGAC | 286 |
| AS32 | GGCGAGGTGGAGCTCTCAGAGGGCGGTGAGGGCCAGC | 288 |
| | GGCACCTTGCATTTCCCTGGGCCTGCTCTGGGCCGGGC | |
| | TGGAGAGGGGTGTGCTGTGCTGCTGTGGAGCCTGCT | |
| AS63 | ATTGAAATGAAAAAACTGTTAAAAAGT | 290 |
| AS34 | AAGATGCGGGCCATCCAGGCCGAGGGTGGGCACGGGC | 292 |
| | AGGCCTGCTGTGGAGGGGCCTGGGGATGGGCACCGGG | |
| | GGACGGGGGCCCCCAGGGGATGCTCACGCATACTCTG | |
| | CCCACCCTGGGCTTCCAGAGCGCCTGGACATGGAGAA | |
| | GAGAAGATGCAGACAGAGCCTGGAGGACTCCGAAAG | |
| | CCTGCGCATCAAGGAGGTGGAGCATA | |
| AS35 | CTGCTGGAGCCCTTCCGCCGCGGTGAGCCCGGGCCCC | 294 |
| | GCGGGCTGCTCTCGGGAAGTTCCCGCGGAGGGGAGGG | |
| | GCCTGGCCGTTCGATCGAGGCTGCACCCGCCACACCTT | |
| | TGCCCTGTTGCCGCAAGAACCCTTGTCGGCCCCAGCCT | |
| | TCCAGATTTTTGCCTCCTAGGGTATTGTTAGTGATCAT | |
| | TCTTCCCAAACTGGATTGTCCAAAACTTGGGTTC | |
| AS36 | CCCTCGGGGCGGAGAACCAAACGGTTAGTTACCCTGC | 296 |
| | GTTCTGGCTGCGCCATACAATGCTGGCATCCTCGTGCC | |
| | GGCCCAGTTCCCTCAGCGCTTCCTCACACAGAGAGGCC | |
| | CCCAAGGCTTGTCAGGGGAGCAGCAGATCCCAGGACA | |
| | GTGACCCTGGGACGGAGCCCTGCAGTCATGCCTCGGG | |
| | CCCCTGCG | |
| AS37 | TGCCACCTCTTCCTGCAGCCCCAGGTTGGCACCCCCCC | 298 |
| | CCCCCACACTGCCAGTGCTCGAGCCCCCAGTGGTCCAC | |
| | CCCACCCTCATGAAAGTTGCCCTGCAGGGCGAAGACC | |
| | TGCGAGAGCTGCGCAGACATGTGCACGCCGACAGCAC | |
| | GGACTTCCTGGCTGTGAAGAGGCTGGTACAGCGCGTG | |
| | TTCCCAGCCTGCACCTGCACCTGCACCAGGCCGCCCTC | |
| | GGAGCAGGAAGGGGCCGTGGGTGGGGAGAGGCCTGT | |
| | GCCCAAGTACCCCCCTCAAGAGGC | |
| AS38 | AAGGAGCTCAAGCTGGAGCAGCAGGTGGGTGGGCAG | 300 |
| | GGCTTGAGAGGGGTGGGCCAAGGGGTGCGTGGCGGCT | |
| | TCGTGACCCTCACTACCCATACCCCGTTCCCCTCCCAG | |
| | GAAGCTGCAGAGCGGGAGTCTAAA | |
| AS39 | GGAGAAATCAGCCAGGAAGAGGTGCCTCCCTCCCGCC | 302 |
| | ACCTGGGCGTCTCATGGGGTGCTGGGGTGTGGGCGGG | |
| | CCTCACCCTCGGGGCCTCTGCACCCCCTAACTCTAGCT | |
| | TCCCCTCAGGTGCTGAGCTACAGCCAGTTGTGTGCTGC | |
| | ATTAGGAGTGACACAAGACAGCCCCGACCCCCCGACT | |
| | TTCCTCAGCACAGGGGAGATCCACGCCTTCCTCAGCTC | |
| | TCCCTCGGGGCGGAGAACCAAACGGTTAGTTACCCTG | |
| | CGTTCTGGCTGCGCCATACAATGCTGGCATCCTCGTGC | |
| | CGGCCCAGTTCCCTCAGCGCTTCCTCACACAGAGAGGC | |
| | CCCCAAGGCTTGTCAGGGGAGCAGCAGATCCCAGGAC | |
| | AGTGACCCTGGGACGGAGCCCTGCAGTCATGCCTCGG | |
| | GCCCCTGCGTAACCTCCACTGTCTCCAGCCCAGGTCTC | |
| | CTTCCTCAGAGGCTATTGCCTCTCGCTCTGACTGGGCT | |
| | CCCTGTGGAGGAAGATGGTTTCGAGCACGCGGGAGCC | |
| AS40 | GACTGCATGCTCAGCGAGGAAGGTGGGCAGGCGCGGC | 304 |
| | GGGGTGGATCCCTCTGCTCCTTAGCTGCCCACACCATT | |
| | GCCTCGGCAGCCCGAGGTCGCTTCCTCTCCAGGCTCTC | |
| | CAATTTCTGTGCCGTAGTTAAAGCGAGCAGGGGCGCC | |
| | CCTTCCTGCACCTGGGAG | |
| AS41 | GAGGCCTTCCAGAGGGCCGCTGGTGAGGGCGGCCCGG | 306 |
| | GCCGCGGTGGGGCACGGCGCGGTGCCAGGGTGTTGCA | |
| | GAGCCCCTTTTGCAGGGCAGGAGCTGGGGAGTGGTTA | |
| | GGACATCAGTCCCTCAGG | |
| AS42 | CCTGAGCCCAGGAGACTGAGCCCAGGTGAGCCCAGGG | 308 |
| | GGCGACCCCGGAAGGGCTGGGGGATCTGGGGTTTGTG | |
| | TGGAGCGCGGGTGGGGCCCAAGGCTTGGCGG | |
| AS43 | GTGCCCTTCCGGGAGCTCAAGAACGTGAGTGTCCTGG | 310 |
| | AGGGGCTCCGTCAAGGCCGGCTTGGGGGTCCCTGTTC | |
| | ATGTCACTGCCCAAGACCTTCCCAGGCCAGGCTCACGC | |
| | CAGTGGATGTGGCAGGTCCCTTCTTGTGTCTGGGGGAT | |
| | CCTGGGCTGTTCCCCCCAGTCAAGAGCAGTATC | |
| AS44 | TTTGTGAGCCTGACTGCCATCCAGATGGCATCGTCGGC | 312 |
| | CACTCCCTGGGGGAGGTGGCCTGTGGCTACGCCGACG | |
| | GCTGCCTGTCCCAGGAGGAGGCCGTCCTCGCTGCCTAC | |
| | TGGAGGGGACAGTGCATCAAAGAAGCCCATCTCCCGC | |
| | CGGGCGCCATGGCAGCCGTGGGCTTGTCCTGGGAGGA | |
| | GTGTAAACAGCGCTGCCCCCCGGGCGTGGTGCCCGCC | |
| | TGCCACAACTCCAAGGACACAGTCACCATCTCGGGAC | |
| | CTCAGGCCCCGGTGTTTGAGTTCGTGGAGCAGC | |
| AS45 | CCAGTTGCCATTAAACCTGGTACAGGGCCGCCCAATA | 314 |
| | ACTCCAGTATACACGGTGGCTCCAAACGTTCAGAGAA | |
| | TTCCTACTGCCGGGATCTACGGGGCCAGTTACGTGCCA | |
| | TTTGCTGCTCCAGCTACAGCCACGATCGCCACACTACA | |
| | GAAGAACGCGGCAGCCGCGGCCGCCATGTATGGAGGA | |
| | TACGCAGGCTACATACCTCAGGCCTTCCCTGCTGCTGC | |
| | CATTCAGGTCCCCATCCCCGACGTCTACCAGACATACT | |
| | GAGGCTGGTGACCAGCACGAAGACAGACCACACAAAC | |
| | ACCACTGAAGGAACGCTTGACTATTTA | |
| AS46 | AAGTGGAACAAGAACTGGACAGCCACACTCGGGGCTC | 316 |
| | TTACAATCAGGGGTCATAAGCTGCTATGTCACCTACCT | |
| | CACCTTCTCAGCTCTGTCCAGCAAACCTGCAGAAGTAG | |
| | TTCTAGA | |
| AS47 | TTCAAGAAGTTCGACGGCCCTTGTGGTGAGCGCGGCGG | 318 |
| | CGGGCGCACGGCTCGAGCTCTGTGGGCGCGCGGCGACA | |
| | GCGTCCTGACTCCTGCCCTCGACCCCCAGACCCCTGTC | |
| | AGGGCGCCCTCCCTGACCCGAGCCGCAGCTGCCGTG | |
| AS48 | GAAAATGCCAGCCTAGTGTTTACAGGATCCAACAGCC | 320 |
| | CCATACCAGCCTGCGAACTGAGTAGTCACCCAGCTCAT | |
| | GGTATCAGTCCTTGGATACCCTCACCTGGAAATGAACA | |
| | TTTCCATGGCATAAAGAAGCAAGTAAAGGCAATAAAA | |
| | GTAGAA | |
| AS49 | CGGCTGACGCAACGGCTGGTCCAGGGCTGGACCCCAA | 322 |
| | TGGAGAACAGGTGGTGTGGCAGGCGAGCGGGTGGGCA | |
| | GCCCGCATCATCCAGCACGAGATGGACCACCTGCAGG | |
| | GCTGCCTGTTTATTGACAAAATGGACAGCAGGACGTTC | |
| | ACAAACGTCTATTGGA | |
| AS50 | GAAAATTCAGGCAACGCCTCGCGTTGGCTGCATGTAC | 324 |
| | CAAGTAGTTCAGACGATTGGCTCGGATGGAAAAAATC | |
| | TTCTGCAATTACTTCCAATTCC | |
| AS51 | GGCATGGAGTGCACCCTGGGGCAGGTGGGTGCCCCGT | 326 |
| | CCCCTCGGAGGGAGGAGGACGGTTGGCGTGGGGGCCA | |
| | CAGCCGATTCAAGGCTGATGTACCAGCACCGCAGGGA | |
| | CCCTGCTGGGGTGGCCAACCTGGCTCTGCCCCCTCCTC | |
| | AGCTCCTCCTGAACAGTCATTATTAGAT | |
| AS52 | AAAGGGAGTGTGGAGAGGCGCTCGGTGAGCCTGGGGC | 328 |
| | ATCCTGCTGAGGGTTGGGCATGGGCAGAGAGGAGCCT | |
| | CCAGCCAGGCATGACCACAGCCAACACAGGCTGCCTC | |
| | TCATTCCACCACAGAGGGTGCCTCCTCCCTGTTTTGCC | |
| | CAAATTACACTGTGGGCTAGGTGGACTACCTCTTGTCA | |
| | GAGCTAAAGAAATCAAGCGAGTGCAGAGGGCAGGGG | |
| | AGAGTTCGCTGCCTGTGAAGGGCCTTCTCACCGTCGCT | |
| | TCGGCTGTCATCGCAGTCCTGTGGGGTAGGCCAAGCG | |
| | AGGTCACAGGAGAAAATGAGGCTCAGCATGAT | |
| AS53 | TTTGGCCTCACCACACTTGCAGGTAGAAGCTCCCACGG | 330 |
| | GACCTCAGGACTGAGGGCAGCCACACACACCAAGTCTG | |
| | GGGACGGTGGCCAGGGGGCTGCCAGGCAGTGTGAGAAG | |
| | CTCCTGGAGCTGGCCCGGGCTACCAGACCCTGGGGGAG | |
| | GAGTACGTCAGCATCATCCAGGTGGACCCATCGCGGAT | |
| | ACATGTGCCCTCCTCGCTGCGCCGTGGCGTGCTGG | |
| AS54 | ATTATTGACAGCGACAAGATAATGGCAGTGTGCATGG | 332 |
| | GGTGCCTGCTCACACGTCATGTGCAATGCCAGGCCATG | |
| | GAGATGCAACAG | |
| AS55 | GATGGCCACTCCTACACATCCAAGGTGAATTGTTTACT | 334 |
| | CCTTCAAGATGGGTTCCATGGCTGTGTGAGCATCACCG | |
| | GGGCAGCTGGAAGAAGAAACCTGAGCATCTTCCTGTT | |
| | CTTGATGCTGTGCAAATTGGAGTTCCATGCTTGT | |
| AS56 | CTACTAAATGCAGAAGATTACCGGTGTGCCATTCATTC | 336 |
| | AAAAGAGATTTATCTTCTTTCCCCCTCCCCCCACCAGG | |
| | CAATGGACAAGTTTTCTCTCTGCTGCATCAACTGCAAT | |
| | CTATGTTTACATGTATTCCTTTTACTACTATTTTTTCA | |
| | AAACAAAGATGTATGGCTTATTTCAAACATCATTTTAC | |
| | TTTGGATATATGGCGGTATT | |
| AS57 | ACAGGGGGCAAAAGCACCTGCTCGGCTCCTGGCCCTC | 338 |
| | AGTCTCTCCCCTCCACTCCATTCTCCACCTACCCACAG | |
| | TGGGTCATTCTGATCACCGAACTG | |
| AS58 | GTGGAGACCCTGGAGAATGCCAACAGCTTCTCCAGCG | 340 |
| | GGATCCAGCCACTCCTCTGTTCCCTGATTGGCCTGGAG | |
| | AATCCCACC | |
| AS59 | GCTGGGGCTGGAACAATTTCCGAAGGTAGTGTTCTCCA | 342 |
| | TGGTCAGAGGCTGGAGTGTGATGCCAGACGTTTTTTTG | |
| | GGTGTGGGACTACAATACTGGCAGAGTGGGAGCACCAT | |
| AS55.1 | GATGGCCACTCCTACACATCCAAGGTGAATTGTTTACT | 386 |
| | CCTTCAAGATGGGTTCCATGGCTGTGTGAGCATCACCG | |
| | GGGCAGCTGGAAGAAGAAACCTGAGCATCTTCCTGTT | |
| | CTTGATGCTGTGCAAATTGGAGTTCCATGCT | |

TABLE 10

| Neoepitope ID | TCGA (%) | SU2C (%) |
|---|---|---|
| AS1 | 28.5 | 2.3 |
| AS2 | 18.5 | N.O. |
| AS3 | 10.4 | 25.6 |
| AS4 | 27.4 | 41.9 |
| AS5 | 18.7 | 9.3 |
| AS6 | 5.1 | 16.3 |
| AS7 | 5.1 | 16.3 |
| AS8 | N.O. | 14.0 |
| AS9 | 1.2 | 18.6 |
| AS10 | 8.9 | 27.9 |
| AS11 | 1.2 | 48.8 |
| AS12 | 0.4 | 34.9 |
| AS13 | 5.7 | 32.6 |
| AS14 | N.O. | 30.2 |
| AS15 | 4.5 | 46.5 |
| AS16 | 0.6 | 18.6 |
| AS17 | N.O. | 37.2 |
| AS18 | 12.6 | 20.9 |
| AS19 | 12.6 | 20.9 |
| AS20 | 0.2 | 16.3 |
| AS21 | N.O. | 11.6 |
| AS22 | 0.2 | 20.9 |
| AS23 | 3.1 | 18.6 |
| AS32 | 57.1 | N.O. |
| AS33 | 47.6 | N.O. |
| AS34 | N.O. | 42.9 |
| AS35 | N.O. | 42.9 |
| AS36 | N.O. | 40.5 |
| AS37 | N.O. | 38.1 |
| AS38 | N.O. | 35.7 |
| AS39 | N.O. | 33.3 |
| AS40 | N.O. | 33.3 |
| AS41 | N.O. | 33.3 |
| AS42 | N.O. | 33.3 |
| AS43 | N.O. | 31 |
| AS44 | N.O. | 28.6 |
| AS45 | N.O. | 26.2 |
| AS46 | N.O. | 26.2 |
| AS47 | N.O. | 23.8 |
| AS48 | N.O. | 23.8 |
| AS49 | N.O. | 23.8 |
| AS50 | N.O. | 23.8 |
| AS51 | N.O. | 23.8 |
| AS52 | 15.9 | 38.1 |
| AS53 | 16 | 9.5 |
| AS54 | 11.9 | N.O. |
| AS55 | 12.1 | N.O. |
| AS56 | 14.7 | N.O. |
| AS57 | 14.9 | N.O. |
| AS58 | 16.6 | N.O. |
| AS59 | 17.6 | N.O. |
| AS63 | 18.0 | |

N.O. not observed

### Example 3: Identification of Additional Neoantigens Using Bioinformatics

Additional neoantigen sequences were identified by further queries as described in Example 2. Table 11 shows the amino acid sequences of the additional neoantigens. Table 12 shows the corresponding polynucleotide sequences.

TABLE 11

| Neoantigen | | | SEQ ID |
|---|---|---|---|
| ID | Gene(s) | Amino acid sequence | NO: |
| P16 | MSMB-NCOA4 | GVPGDSTRRAVRRMNTF | 343 |
| P17 | MSMB-NOCA4 | GVPGDSTRRAVRRMNTF | 343 |
| P19 | TMEM222- | WTPIPVLTRWPLPHPPPWRRATSCRM | 347 |
| | LOC644961 | ARSSPSATSGSSVRRRCSSLPSWVWNL | |
| | | AASTRPRSTPS | |
| P22 | SLC45A3-ELK4 | SLYHREKQLIAMDSAI | 349 |
| P27 | FAM126B- | LHPQRETFTPRWSGANYWKLAFPVGA | 351 |
| | ORC2 | EGTFPAAATQRGVVRPA | |
| P35 | TMPRSS2-ERG | NSKMALNSLNSIDDAQLTRIAPPRSHC | 353 |
| | | CFWEVNAP | |
| P37 | TSTD1-F11R | MAGGVLRRLLCREPDRDGDKGASRE | 355 |
| | | ETVVPLHIGDPVVLPGIGQCYSALF | |
| P46 | TP53 (R213D) | DDRNTFDIVWWCPMSRLRLALTVPPS | 357 |
| | | TTTTCVTVPAWAA | |
| P48 | AR.p.H875Y | VQPIARELYQFTFDLLI | 359 |
| P50 | AR (W742C) | QMAVIQYSCMGLMVFAM | 361 |
| P56 | SPOP (F102C) | PKSEVRAKCKFSILNAK | 363 |
| P58 | AR (Q903H) | MMAEIISVHVPKILSGK | 365 |
| P59 | FOXA1 (F254V) | LHPDSGNMVENGCYLRR | 367 |
| P60 | FOXA1.p.F266L | CYLRRQKRLKCEKQPGA | 369 |
| P61 | FOXA1.p.R261G | MFENGCYLGRQKRFKCE | 371 |
| P73 | TP53 (G266E) | DSSGNLLERNSFEVRV | 373 |
| P76 | AR-V3 | VFFKRAAEGFFRMNKLKESSDTNPKPY | 375 |
| | | CMAAPMGLTENNRNRKKSYRETNLK | |
| | | AVSWPLNHT | |
| P77 | AR-V3 | VFFKRAAEGFFRMNKLKESSDTNPKP | 375 |
| | | YCMAAPMGLTENNRNRKKSYRETNL | |
| | | KAVSWPLNHT | |
| P82 | AR-V7 | YEAGMTLGEKFRVGNCKHLKMTRP | 379 |
| P87 | AR-Intron | YEAGMTLGGKILFFLFLLLPLSPFSLIF | 381 |
| P97 | FOXRED2- | GYLRMQGLMAQRLLLR | 383 |
| | TXN2 | | |
| P98 | TP53 (R213D) | DDRNTFDIVWWCPMSRLRLALTVPPS | 357 |
| | | TTTTCVTVPAWAA | |

TABLE 12

| Neoantigen | | | SEQ ID |
|---|---|---|---|
| ID | Gene(s) | Nucleotide sequence | NO: |
| P16 | MSMB-NCOA4 | GGAGTTCCAGGAGATTCAACCAGGA | 344 |
| | | GAGCAGTGAGGAGAATGAATACCTTC | |
| P17 | MSMB-NOCA4 | GGAGTTCCAGGAGATTCAACCAGGA | 344 |
| | | GAGCAGTGAGGAGAATGAATACCTTC | |
| P19 | TMEM222- | TGGACGCCCATCCCGGTGCTCACGA | 348 |
| | LOC644961 | GATGGCCACTACCACATCCTCCTCCC | |
| | | TGGAGAAGAGCTACAAGCTGCCGGA | |
| | | TGGCCAGGTCATCACCATCAGCAAC | |
| | | AAGCGGTTCCAGTGTCCGGAGGCGC | |
| | | TGTTCCAGCCTTCCTTCCTGGGTATG | |
| | | GAATCTTGCGGCATCCACGAGACCA | |
| | | CGTTCAACTCCATCATGAA | |
| P22 | SLC45A3-ELK4 | TCCCTCTACCACCGGGAGAAGCAGC | 350 |
| | | TCATTGCTATGGACAGTGCTATC | |
| P27 | FAM126B- | CTTCATCCTCAGAGGGAAACATTCAC | 352 |
| | ORC2 | TCCCCGGTGGTCGGGCGCGAATTACT | |
| | | GGAAATTGGCTTTTCCCGTTGGGGCC | |
| | | GAAGGTACCTTCCCTGCGGCGGCGA | |
| | | CTCAGCGGGGTGTCGTTCGGCCGGC | |
| | | GTG | |
| P35 | TMPRSS2-ERG | AACAGCAAGATGGCTTTGAACTCATT | 354 |
| | | AAACTCCATTGATGATGCACAGTTGA | |
| | | CAAGAATTGCCCCTCCAAGATCTCAT | |
| | | TGCTGTTTCTGGGAAGTAAACGCTCC | |
| | | T | |
| P37 | TSTD1-F11R | ATGGCTGGAGGAGTCCTTCGGCGGC | 356 |
| | | TGTTGTGTCGGGAGCCTGATCGCGAT | |
| | | GGGGACAAAGGCGCAAGTCGAGAGG | |
| | | AAACTGTTGTGCCTCTTCATATTGGC | |
| | | GATCCTGTTGTGCTCCCTGGCATTGG | |
| | | GCAGTGTTACAGTGCACTCTTCT | |
| P46 | TP53 (R213D) | GATGACAGAAACACTTTCGACATAG | 358 |
| | | TGTGGTGGTGCCCTATGAGCCGCCTG | |
| | | AGGTTGGCTCTGACTGTACCACCATC | |
| | | CACTACAACTACATGTGTAACAGTTC | |
| | | CTGCATGGGCGGCATGA | |
| P48 | AR.p.H875Y | GTGCAGCCTATTGCGAGAGAGCTGC | 360 |
| | | ATCAGTTCACTTTTGACCTGCTAATC | |
| P50 | AR (W742C) | CAGATGGCTGTCATTCAGTACTCCTG | 362 |
| | | CATGGGGCTCATGGTGTTTGCCATG | |
| P56 | SPOP (F102C) | CAAAGAGTGAAGTTCGGGCAAAATT | 364 |
| | | CAAATGCTCCATCCTGAATGCCAAG | |
| P58 | AR (Q903H) | ATGATGGCAGAGATCATCTCTGTGCA | 366 |
| | | CGTGCCCAAGATCCTTTCTGGGAAA | |
| P59 | FOXA1 (F254V) | CTGCACCCGGACTCCGGCAACATGG | 368 |
| | | TCGAGAACGGCTGCTACTTGCGCCGC | |
| P60 | FOXA1.p.F266L | TGCTACTTGCGCCGCCAGAAGCGCTT | 370 |
| | | GAAGTGCGAGAAGCAGCCGGGGGCC | |
| P61 | FOXA1.p.R261G | ATGTTCGAGAACGGCTGCTACTTGGG | 372 |
| | | CCGCCAGAAGCGCTTCAAGTGCGAG | |
| P73 | TP53 (G266E) | GACTCCAGTGGTAATCTACTGGAAC | 374 |
| | | GGAACAGCTTTGAGGTGCGTGTT | |
| P76 | AR-V3 | GTCTTCTTCAAAAGAGCCGCTGAAG | 376 |
| | | GATTTTTCAGAATGAACAAATTAAA | |
| | | AGAATCATCAGACACTAACCCCAAG | |
| | | CCATACTGCATGGCAGCACCAATGG | |
| | | GACTGACAGAAAACAACAGAAATAG | |
| | | GAAGAAATCCTACAGAGAAACAAAC | |
| | | TTGAAAGCTGTCTCATGGCCTTTGAA | |
| | | TCATACT | |
| P77 | AR-V3 | GTCTTCTTCAAAAGAGCCGCTGAAG | 376 |
| | | GATTTTTCAGAATGAACAAATTAAA | |
| | | AGAATCATCAGACACTAACCCCAAG | |
| | | CCATACTGCATGGCAGCACCAATGG | |
| | | GACTGACAGAAAACAACAGAAATAG | |
| | | GAAGAAATCCTACAGAGAAACAAAC | |
| | | TTGAAAGCTGTCTCATGGCCTTTGAA | |
| | | TCATACT | |
| P82 | AR-V7 | TATGAAGCAGGGATGACTCTGGGAG | 380 |
| | | AAAAATTCCGGGTTGGCAATTGCAA | |
| | | GCATCTCAAAATGACCAGACCC | |
| P87 | AR-Intron | TATGAAGCAGGGATGACTCTGGGAG | 382 |
| | | GTAAGATACTTTTCTTTCTCTTCCTCC | |
| | | TCCTTCCTCTCTCCCCCTTCTCCCTCA | |
| | | TTTTC | |
| P97 | FOXRED2- | GGGTACCTGAGGATGCAGGGACTCA | 384 |
| | TXN2 | TGGCTCAGCGACTTCTTCTGAGG | |
| P98 | TP53 (R213D) | GATGACAGAAACACTTTCGACATAG | 358 |
| | | TGTGGTGGTGCCCTATGAGCCGCCTG | |
| | | AGGTTGGCTCTGACTGTACCACCATC | |
| | | CACTACAACTACATGTGTAACAGTTC | |
| | | CTGCATGGGCGGCATGA | |

### Example 4: HLA Binding Predictions

The amino acid sequences of the neoantigens identified using the various approaches as described in Example 3 were split into all possible unique, contiguous 9 mer amino acid fragments and HLA binding predictions to six common HLA alleles (HLA-A*01:01, HLA-A*02:01, HLA-A*03:01, HLAA*24:02, HLA-B*07:02, HLA-B*08:01) were performed for each of these 9mers using netMHCpan4.0. Several 9 mer fragments were selected for further analysis based on ranking by likelihood of binding to one or more of the tested HLA alleles and their prevalence in prostate cancer patients.

Table 13 shows the amino acid sequences of select 9 mer fragments and their neoantigen origin. Table 14 shows the prevalence of neoantigens in the analyzed cohorts.

TABLE 13

| | | | Amino acid | |
|---|---|---|---|---|
| Neoantigen | | | sequence or 9- | SEQ ID NO: |
| ID | Gene(s) | Type | mer | of the 9mer |
| P16 | MSMB-NCOA4 | Fusion | STRRAVRRM | 387 |
| P17 | MSMB-NOCA4 | Fusion | RAVRRMNTF | 388 |
| P19 | TMEM222- | Fusion | IPVLTRWPL | 389 |
| | LOC644961 | | | |
| P22 | SLC45A3-ELK4 | Fusion | QLIAMDSAI | 390 |
| P27 | FAM126B-ORC2 | Fusion | FPVGAEGTF | 391 |
| P35 | TMPRSS2-ERG | Fusion | NSKMALNSL | 392 |
| P37 | TSTD1-F11R | Fusion | GVLRRLLCR | 393 |
| P46 | TP53 (R213D) | Frameshift | CPMSRLRLA | 394 |
| | | Mutation | | |
| P48 | AR.p.H875Y | Missense | YQFTFDLLI | 395 |
| | | Mutation | | |
| P50 | AR (W742C) | Missense | IQYSCMGLM | 396 |
| | | Mutation | | |
| P56 | SPOP (F102C) | Missense | RAKCKFSIL | 397 |
| | | Mutation | | |
| P58 | AR (Q903H) | Missense | HVPKILSGK | 398 |
| | | Mutation | | |
| P59 | FOXA1 (F254V) | Missense | NMVENGCYL | 399 |
| | | Mutation | | |
| P60 | FOXA1.p.F266L | Missense | YLRRQKRLK | 400 |
| | | Mutation | | |
| P61 | FOXA1.p.R261G | Missense | CYLGRQKRF | 401 |
| | | Mutation | | |
| P73 | TP53 (G266E) | Missense | LLERNSFEV | 402 |
| | | Mutation | | |
| P76 | AR-V3 | Splice Variant | YCMAAPMGL | 403 |
| P77 | AR-V3 | Splice Variant | FFKRAAEGF | 404 |
| P82 | AR-V7 | Splice Variant | RVGNCKHLK | 405 |
| P87 | AR-Intron | Splice Variant | FLFLLLPLS | 406 |
| P97 | FOXRED2-TXN2 | Fusion | LMAQRLLLR | 407 |
| P98 | TP53 (R213D) | Frameshift | IVWWCPMSR | 408 |
| | | Mutation | | |

TABLE 14

| Neoantigen | | Prevalence | |
|---|---|---|---|
| ID | Gene | TCGA | SU2C |
| P16 | MSMB-NCOA4 | 27.16% | 23.25% |
| P17 | MSMB-NOCA4 | 27.16% | 23.25% |
| P19 | TMEM222-LOC644961 | N.O. | 13.95% |
| P22 | SLC45A3-ELK4 | 17.71% | 13.95% |
| P27 | FAM126B-ORC2 | 5.11% | 18.60% |
| P35 | TMPRSS2-ERG | 2.75% | 11.62% |
| P37 | TSTD1-F11R | 16.33% | 9.30% |
| P46 | TP53 (R213D) | N.O. | 1% |
| P48 | AR.p.H875Y | N.O. | 1% |
| P50 | AR (W742C) | N.O. | 1.25% |
| P56 | SPOP (F102C) | 0.40% | 2.00% |
| P58 | AR (Q903H) | N.O. | 1.00% |
| P59 | FOXA1 (F254V) | 0.20% | 1.00% |
| P60 | FOXA1.p.F266L | 0.20% | 1% |
| P61 | FOXA1.p.R261 G | 0.20% | 1% |
| P73 | TP53 (G266E) | N.O. | 1% |
| P76 | AR-V3 | Present | Present |
| P77 | AR-V3 | Present | Present |
| P82 | AR-V7 | Present | Present |
| P87 | AR-Intron | Present | Present |
| P97 | FOXRED2-TXN2 | 3.74% | 11.62% |
| P98 | TP53 (R213D) | 0.00% | 1.00% |

NO: not observed;
Present: AR splice variants were expressed at variable levels and hence prevalence was not determined

### Example 5: Immunogenicity Assessment of Neoantigens

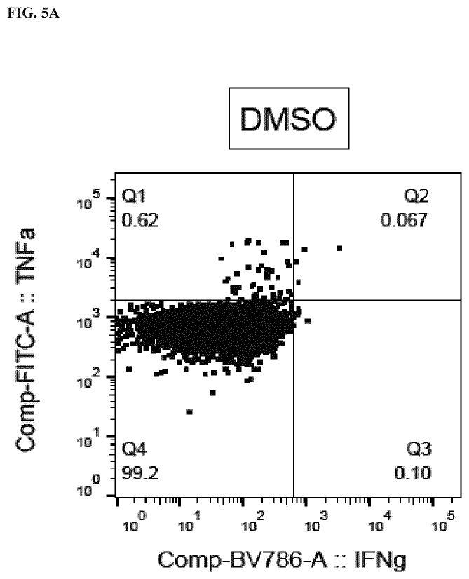

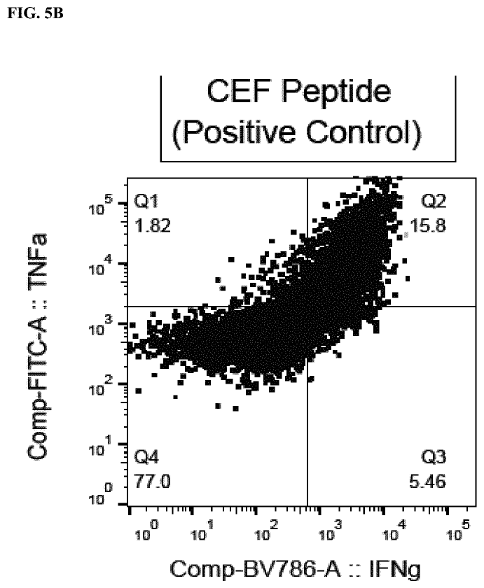

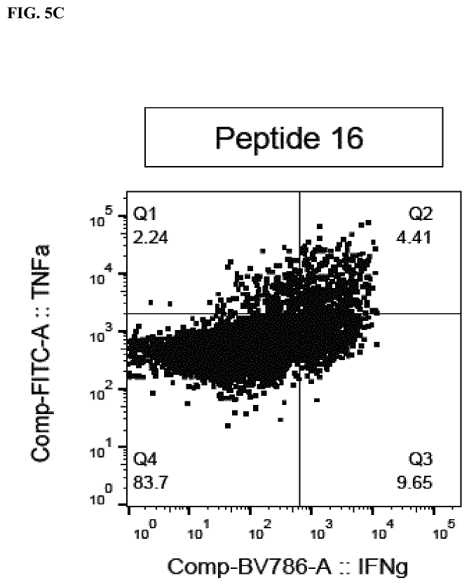

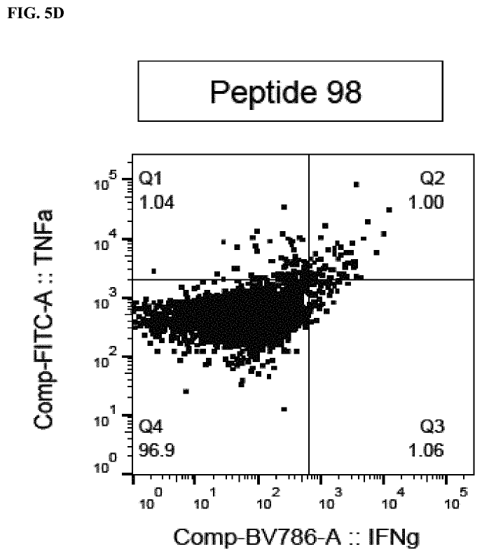

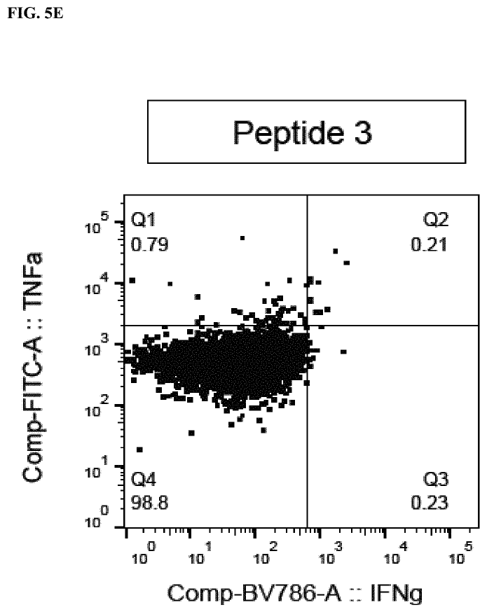

The 9 mer fragments shown in Table 13 were assessed for their ability to activate T cells using the Patient PBMC restimulation assay described in Example 1 using TNFα and IFNγ production by CD8⁺ T cells as a readout. Self-antigens shown in Table 15 were also used in the assays. FIG. 5A, FIG. 5B, FIG. 5C, FIG. 5D and FIG. 5E show flow cytometry dot plots depicting TNFα⁺IFNγ⁺CD8⁺ T cell frequencies in PBMC samples after no stimulation (DMSO negative control) (FIG. 5A), after stimulation with CEF peptide (positive control (FIG. 5B), after stimulation with P16 (FIG. 5C), after stimulation with P98 (FIG. 5D), and after stimulation with P3 (FIG. 5E). Table 16 shows the maximum frequency of TNFα⁺IFNγ⁺CD8⁺ T cells and maximum fold change over negative control for each peptide analyzed, indicating the highest frequency of TNFα⁺IFNγ⁺CD8⁺ T cells and resulting fold change across the PBMC donors evaluated for the peptide. All neoantigens evaluated were found to stimulate CD8⁺ T cells. FIG. 6 shows the number of prostate cancer patients whose PBMC samples demonstrated a positive immune response to the specified neoantigens. PBMCs from ten patients were evaluated.

TABLE 15

| Peptide | | Amino Acid | SEQ ID |
|---|---|---|---|
| ID | Gene name | Sequence of the 9-mer | NO: |
| P3 | ERG | KLSRALRYY | 421 |
| P6 | FOLH1 | MVFELANSI | 422 |
| P7 | ERG | ILFQNIDGK | 423 |
| P9 | FOLH1 | KIVIARYGK | 424 |
| P92 | ERG | FLLELLSDS | 425 |

TABLE 16

| | | Maximum frequency of | |
|---|---|---|---|
| Peptide | Maximum fold | TNFα⁺IFNγ⁺CD8⁺ T cells | |
| Name | change over | (Percent) | |
| Negative | negative control | 0.011-0.8 (depending | Immunogenic |
| control | n/a | on patient) | n/a |
| P16 | 65.82 | 4.620 | Yes |
| P17 | 2.17 | 0.130 | Yes |
| P19 | 5.00 | 0.480 | Yes |
| P22 | 5.00 | 0.120 | Yes |
| P27 | 3.43 | 0.430 | Yes |
| P35 | 2.67 | 0.064 | Yes |
| P37 | 3.13 | 1.160 | Yes |
| P46 | 2.33 | 0.140 | Yes |
| P48 | 2.33 | 0.220 | Yes |
| P50 | 5.14 | 0.190 | Yes |
| P56 | 11.57 | 1.620 | Yes |
| P58 | 19.18 | 5.370 | Yes |
| P59 | 10.75 | 3.010 | Yes |
| P60 | 2.08 | 0.340 | Yes |
| P61 | 2.27 | 0.084 | Yes |
| P73 | 2.97 | 0.110 | Yes |
| P76 | 2.30 | 0.170 | Yes |
| P77 | 3.24 | 0.160 | Yes |
| P82 | 3.46 | 0.970 | Yes |
| P87 | 3.24 | 0.120 | Yes |
| P97 | 4.55 | 0.160 | Yes |
| P98 | 14.93 | 1.000 | Yes |

Maximum frequency refers to the greatest frequency of TNFα⁺IFNγ⁺CD8⁺ T cells among all tested PBMC donors

### Example 6: Binding of Neoantigens to HLA

Binding of select neoepitopes to HLA-A*01:01, HLA-A*02:01, HLA-A*03:01, HLA-A*24:02, HLA-B*07:02 and HLA-B*08:01 was evaluated using the assay described in Example 1. The results of binding the various neoantigens to HLA is shown in Table 17. Each HLA allele tested had a corresponding positive control (Pos) and a negative control (Neg) peptide against which the peptide of interest was exchanged. An exchange rate of 100% with Pos, thus means that the peptide of interest has the same binding affinity to the HLA allele as the positive control peptide. As a criterion for further evaluation, peptides with an exchange rate of at least 1000 with the corresponding Pos peptide for at least one of the 6 HLA alleles that we considered for further evaluation. The exchange rates with the allele specific Pos peptides, of the 24 neoantigens so identified are summarized below in Table 17. Higher percentages correspond to stronger binding to the HLA allele.

TABLE 17

| Peptide | HLA- | HLA- | HLA- | HLA- | HLA- | HLA- |
|---|---|---|---|---|---|---|
| Name | A*01:01 | A*02:01 | A*03:01 | A*24:02 | B*07:02 | B*08:01 |
| P16 | 5.5 | 10.4 | 6.1 | 0.8 | 13.7 | 6.2 |
| P17 | 4.9 | 9.3 | 3.8 | 0.1 | 38.7 | 9 |
| P19 | 5.2 | 8.2 | 4.1 | 0.1 | 91.3 | 14.5 |
| P22 | 4.4 | 46.9 | 4.8 | 0 | 8.3 | 3.9 |
| P27 | 4.8 | 10 | 4.5 | 0.7 | 7.8 | 6.4 |
| P35 | 2.5 | 12.5 | 4.2 | 0.2 | 12.1 | 13.7 |
| P37 | 1.4 | 12.5 | 6.4 | −0.1 | 9.4 | 3.3 |
| P46 | 2.4 | 14.4 | 10.3 | −0.4 | 64.8 | 14.5 |
| P48 | 4.3 | 99.3 | 4.4 | 0.1 | 12.8 | 8.9 |
| P50 | 2.3 | 8.8 | 5.5 | 0.1 | 10.4 | 4.7 |
| P56 | 2.6 | 8.4 | 6.8 | 0.5 | 82.5 | 38 |
| P58 | 2.5 | 11.8 | 32.5 | 0.2 | 9.7 | 5.3 |
| P59 | 2.2 | 11.9 | 4.5 | −0.5 | 7.5 | 5.5 |
| P60 | 3.1 | 7.9 | 36.6 | 0.9 | 6.1 | 10.2 |
| P61 | 1.7 | 5.8 | 2.1 | 90.3 | 9.6 | 3.6 |
| P73 | 2.1 | 89.2 | 3.5 | 0.5 | 8.7 | 2.6 |
| P76 | 0.1 | 85.9 | 6 | −0.5 | 91.5 | 8.5 |
| P77 | 1.9 | 9.6 | 2.8 | 1.7 | 14.2 | 3.4 |
| P82 | 1.5 | 6.3 | 58.1 | −0.1 | 12.9 | 1.4 |
| P87 | 1.1 | 64 | 2.4 | 0.2 | 5 | 3.1 |
| P97 | 2.5 | 4.6 | 39 | 0.1 | 7 | 2.7 |
| P98 | 2.5 | 7.9 | 51.1 | −0.1 | 6.7 | 2.4 |

### Example 7: MHC I-Peptide Complex Profiling of Prostate Cancer Tissues Identified Unique MHC I-Presented Peptides in Prostate Cancer

MHC I-peptide complexes were isolated from samples of 11 human prostate cancer and peptides presented by MHC I were identified using unbiased mass spectrometry. At collection, the subjects were diagnosed with grade 7 adenocarcinoma or stromal sarcoma with two subject having invasive adenocarcinoma.

Frozen human prostate cancer tissues with HLA-A*02:01, HLA-A*03:03, HLA-B*27:0 and HLA-B*08:01 haplotypes were mechanically disrupted in non-ionic detergent including protease inhibitors and processed. A pan-MHC allele monoclonal antibody was used to immunopurify MHC I-peptide complexes from the samples. After acid elution, recovery of the MHC I-peptide complexes was assessed by ELISA and recovered peptides desalted and subjected to LC-MS/MS analyses.

The raw LC-MS/MS data files from prostate tumors were analyzed to search against the neoantigen database that was created from corresponding RNAseq data obtained from the 11 human prostate cancer samples. These peptides had a theoretical mass for parent ions (MS1) and a list of theoretical fragment ions (MS2). A list of MS1 ions that had triggered MS2 scans were searched against the theoretical list of peptides and matched by mass. All theoretical peptides within a set MS1 ppm mass accuracy (5 ppm) then had their in silico MS2 spectrum compared to the empirical MS2 for that parent ion (peptide spectral matches or PSMs). A score was computed based on how closely the empirical spectrum matched the theoretical spectrum. Each LC-MS/MS run (one file per tumor sample) produced thousands of PSMs. However, the vast majority of these peptides were canonical sequences that were found in the human reference database (Swissprot). These were filtered out and peptides of interest (putative neoantigens) were compiled. From this list, peptides that had sufficient evidence for being positive were selected.

Table 18 shows the amino acid sequences of the peptides identified in complex with MHC I using LC-MS/MS and the gene origin of the peptides.

Table 19 shows the amino acid sequences of the corresponding longer neoantigens of the peptides identified in complex with MHC I using LC/MS/MS.

Table 20 shows the polynucleotide sequences encoding the corresponding longer neoantigens.

The MHC I complexed peptides described herein confirmed the expression, processing, and presentation of immunogenic epitopes specific to prostate cancer aberrant gene alterations. Evaluation of RNAseq databases mapped the identified MHC I complexed peptides within longer aberrant transcripts present in prostate cancer. Hence, these data identified prostate cancer neoantigens that contained at least one MHC class I epitope that is immunologically relevant and capable of eliciting an adaptive T cell response.

TABLE 18

| | | SEQ | | |
|---|---|---|---|---|
| Neoantigen | Amino acid | ID | | |
| ID | sequence | NO: | Gene | type |
| MS1 | VTFLKPCFLL | 426 | TTLL7 | Alternative 5′ SS |
| MS2 | TDIVKQSV | 427 | CHD7 | Alternative Last Exon |
| MS3 | SPAFPKPVRP | 428 | TESK1 | Alternative Last Exon |
| MS4 | SYFSLTNIFNFV | 429 | PPIP5K2 | Alternative Last Exon |
| MS5 | EFSPETCAFRLS | 430 | SRPK2 | Alternative Last Exon |
| MS6 | FLSRALRAL | 431 | SOAT1 | Alternative 5′ SS |
| MS7 | KKDLELIL | 432 | PDE4D | Alternative Last Exon |
| MS8 | KLQKNCLL | 433 | ZYG11A | Exon Skip |
| MS9 | SALSGNSWV | 434 | SYNE2 | Alternative Last Exon |
| MS10 | TVRAILL | 435 | USP21 | Intron Retention |
| MS11 | GSLHFHEVLK | 436 | TDG | Novel Cassette |

TABLE 19

| Neoantigen | | | Peptide |
|---|---|---|---|
| ID | Gene ID | Peptide Sequence | SEQ ID |
| MS1 | TTLL7 | HYKLIQQPISLFSITDRLHKTFSQLPSVHLC | 437 |
| | | SITFQWGHPPIFCSTNDICVTANFCISVTFLK | |
| | | PCFLLHEASASQ | |
| MS2 | CHD7 | WTDIVKQSVSTNCISIKKGSYTKLFSLVFLI | 438 |
| | | FCWPLIIQL | |
| MS3 | TESK1 | RTALTHNQDFSIYRLCCKRGSLCHASQARS | 439 |
| | | PAFPKPVRPLPAPITRITPQLGGQSDSSQPLL | |
| | | TTGRPQGWQDQALRHTQQASPASCATITIPI | |
| | | HSAALGDHSGDPGPAWDTCPPLPLTTLIPR | |
| | | APPPYGDSTARSWPSRCGPLG | |
| MS4 | PPIP5K2 | LRYGALCNVSRISYFSLTNIFNFVIKSLTAIF | 440 |
| | | TVKF | |
| MS5 | SRPK2 | RKERNIRKSESTLRLSPFPTPAPSGAPAAAQ | 441 |
| | | GKVVRVPGPAGGLVPRDAGARLLPPAGGP | |
| | | GGGAAAGEGRAGRGRFPSITEPRPRDLPPR | |
| | | VATGRRAGGRRKGAGQGVRTRPLPASWPG | |
| | | GRGPFRKGPRRLPLGSGPPAAGVQRLRCSH | |
| | | LSRGPRRRRGRVCGRACVSPPLPPRPPPVGL | |
| | | SAENLSWLSSGLPRACSWREFSPETCAFRLS | |
| | | GLDSKLSARVERDLGALRAPGSRAAQGGG | |
| | | RVRGSRSEWKTRPWRPPPAWPLTRAGGPL | |
| | | PKNPFLESCSETAQRRRVFSFSTPLS | |
| MS6 | SOAT1 | YAYKDFLWCFPFSLVFLQEIQICCHVSCLC | 442 |
| | | CICCSTRICLGCLLELFLSRALRALHVLWNG | |
| | | FQLHCQ | |
| MS7 | PDE4D | SINKATITGKKDLELILHVSRKKPFLPRVNI | 443 |
| | | TPTPISCCNLKMLKKFFLLYIIISIIDLTNCL | |
| | | SCYLEHFYRFTFFTDVHYF | |
| MS8 | ZYG11A | TMPAILKLQKNCLLSL | 444 |
| MS9 | SYNE2 | PYYSALSGNSWVPSTLESDPFGYVFSPLAT | 445 |
| | | RPALNDQESILWPTLTSVVSCALSCPSLNLP | |
| | | ENWLTLITGGMKGGKKMKFTFRH | |
| MS10 | USP21 | GLRNLGNTVRAILLSFLSKRNVKWCWGW | 446 |
| | | GKPTSLGKACGRRALKLF | |
| MS11 | TDG | MEAENAGSLHFHEVLKMGHVKF | 447 |

TABLE 20

| Neo- | | | |
|---|---|---|---|
| antigen | Gene | SEQ ID | DNA |
| ID | ID | Polynucleotide sequence | NO: |
| MS1 | TTLL7 | CACTACAAATTAATTCAACAACCCATATCCCTCTT | 448 |
| | | CTCCATCACTGATAGGCTCCATAAGACGTTCAGTC | |
| | | AGCTGCCCTCGGTCCATCTCTGCTCAATCACCTTCC | |
| | | AGTGGGGACACCCGCCCATTTTCTGCTCAACAAAT | |
| | | GATATCTGTGTCACGGCCAACTTCTGCATCTCGGTC | |
| | | ACATTCCTTAAACCGTGCTTCCTCCTACATGAGGCA | |
| | | TCTGCCTCACAG | |
| MS2 | CHD7 | TGGACTGATATAGTTAAGCAGTCTGTAAGTACAA | 449 |
| | | ACTGCATTTCTATCAAGAAAGGTAGCTATACAAAA | |
| | | CTGTTTTCCTTAGTCTTTCTTATTTTCTGTTGGCCAT | |
| | | TAATTATTCAGTTG | |
| MS3 | TESK1 | AGGACCGCCCTGACACACAATCAGGACTTCTCTA | 450 |
| | | TCTACAGGCTCTGTTGCAAGAGGGGGTCCCTCTGC | |
| | | CACGCTTCCCAGGCCAGATCCCCGGCTTTCCCGAA | |
| | | GCCGGTCAGACCTCTTCCTGCCCCCATCACCAGAAT | |
| | | CACCCCCCAACTGGGGGGACAATCTGACTCGAGTC | |
| | | AACCCCTTCTCACTACGGGAAGACCTCAGGGGTGG | |
| | | CAAGATCAAGCTCTTAGACACACCCAGCAAGCCAG | |
| | | TCCTGCCTCTTGTGCCACCATCACCATTCCCATCCA | |
| | | CTCAGCTGCCCTTGGTGACCACTCCGGAGACCCTG | |
| | | GTCCAGCCTGGGACACCTGCCCGCCGCTGCCGCTC | |
| | | ACTACCCTCATCCCCCGAGCTCCCCCGCCGTATGGA | |
| | | GACAGCACTGCCAGGTCCTGGCCCTCCCGCTGTGG | |
| | | GCCCCTCGGC | |
| MS4 | PPIP5 | CTTCGCTATGGTGCCTTATGCAATGTAAGTAGAA | 451 |
| | K2 | TAAGTTATTTCAGTCTAACAAATATATTTAATTTTG | |
| | | TAATTAAATCACTAACTGCTATTTTTACTGTGAAAT | |
| | | TT | |
| MS5 | SRPK2 | CGAAAAGAGAGAAACATCCGAAAAAGTGAGTCC | 452 |
| | | ACGCTGCGCCTGTCCCCGTTCCCCACCCCCGCCCCG | |
| | | TCGGGGGCGCCCGCGGCCGCGCAGGGGAAAGTTGT | |
| | | CCGGGTCCCCGGGCCGGCGGGCGGGCTGGTCCCCC | |
| | | GGGACGCTGGCGCTCGGCTCCTGCCCCCGGCGGGC | |
| | | GGCCCGGGGGGAGGGGCGGCGGCGGGGGAGGGGC | |
| | | GCGCGGGCCGCGGCCGGTTCCCTAGCATCACGGAG | |
| | | CCTCGACCCCGCGACCTCCCGCCCCGGGTCGCCAC | |
| | | CGGCCGGCGGGCGGGAGGCCGGCGGAAAGGCGCC | |
| | | GGGCAGGGCGTGCGCACCCGTCCCTTGCCCGCGAG | |
| | | CTGGCCCGGGGGTCGCGGCCCTTTCCGGAAGGGGC | |
| | | CCCGGCGTCTGCCGCTGGGCTCCGGCCCGCCCGCT | |
| | | GCGGGAGTGCAGCGGCTGCGTTGCTCCCACCTGAG | |
| | | CCGCGGGCCGAGGAGGCGGAGGGGCCGAGTGTGC | |
| | | GGGAGGGCGTGTGTCTCGCCTCCCCTTCCTCCCCGG | |
| | | CCCCCGCCTGTCGGCCTTTCTGCTGAGAACCTAAGC | |
| | | TGGTTGTCAAGTGGTTTGCCTCGGGCGTGTTCCTGG | |
| | | CGCGAGTTCAGCCCCGAGACCTGTGCGTTTCGGCT | |
| | | CTCGGGTTTGGATTCGAAACTTTCCGCTCGGGTTGA | |
| | | GCGTGACTTGGGTGCGCTGCGGGCGCCGGGGTCGC | |
| | | GGGCTGCGCAGGGCGGTGGGCGTGTGCGCGGGAG | |
| | | CCGGTCGGAGTGGAAAACGCGCCCGTGGCGGCCAC | |
| | | CTCCAGCCTGGCCGCTCACCCGAGCAGGGGGGCCG | |
| | | CTGCCCAAGAACCCTTTCCTGGAGAGCTGCTCCGA | |
| | | GACCGCACAGCGCCGCCGCGTCTTCTCCTTTTCCAC | |
| | | TCCTCTCTCC | |
| MS6 | SOAT | TATGCTTACAAGGACTTTCTCTGGTGTTTTCCTTT | 453 |
| | 1 | TTCTTTAGTTTTTCTCCAAGAGATTCAAATCTGCTG | |
| | | CCATGTTAGCTGTCTTTGCTGTATCTGCTGTAGTAC | |
| | | ACGAATATGCCTTGGCTGTTTGCTTGAGCTTTTTCT | |
| | | ATCCCGTGCTCTTCGTGCTCTTCATGTTCTTTGGAA | |
| | | TGGCTTTCAACTTCATTGTCAA | |
| MS7 | PDE4 | TCCATCAACAAAGCCACCATAACAGGTAAGAAA | 454 |
| | D | GATCTGGAGCTTATTCTTCATGTGTCTAGGAAGAA | |
| | | ACCATTTCTGCCAAGAGTCAATATAACACCAACAC | |
| | | CAATTTCATGCTGCAATTTGAAAATGTTAAAGAAA | |
| | | TTCTTTCTTCTCTACATTATCATTTCTATCATTGATC | |
| | | TCACAAATTGTCTAAGCTGTTATTTGGAACATTTTT | |
| | | ACCGATTTACGTTTTTTACTGATGTACATTATTTT | |
| MS8 | ZYG11 | ACCATGCCTGCTATTTTAAAGTTACAGAAGAATT | 455 |
| | A | GTCTTCTCTCCTTA | |
| MS9 | SYNE | CCATACTACAGCGCACTGTCAGGTAACAGCTGGG | 456 |
| | 2 | TTCCCAGCACCCTGGAAAGTGACCCGTTTGGCTAT | |
| | | GTTTTTAGCCCCTTAGCAACACGGCCAGCTCTCAAT | |
| | | GACCAAGAGTCCATCTTGTGGCCGACCCTGACTTCT | |
| | | GTGGTTTCCTGTGCTCTATCCTGCCCATCTCTTAAC | |
| | | TTACCTGAGAATTGGCTCACTCTCATCACAGGTGG | |
| | | AATGAAAGGGGGAAAAAAAATGAAATTCACATTC | |
| | | AGACAC | |
| MS10 | USP21 | GGCCTTCGAAACCTGGGAAACACGGTGAGAGCT | 457 |
| | | ATTCTCCTATCTTTCCTCTCTAAAAGGAATGTGAAA | |
| | | TGGTGCTGGGGGTGGGGAAAACCCACGAGCTTGGG | |
| | | GAAGGCATGTGGAAGGAGAGCTCTGAAGCTCTTC | |
| MS11 | TDG | ATGGAAGCGGAGAACGCGGGCAGTTTGCATTTTC | 458 |
| | | ATGAAGTGCTCAAAATGGGACATGTGAAATTC | |

### Example 8 Expression Profiling of Prostate Neoantigens in Tumor and Normal Tissues

The identified prostate neoantigens were profiled for their expression in about 90 FFPE tissue samples from prostate cancer (adenocarcinoma, clinical stages II-IV, Gleason score 8-9, subjects were treatment naïve or treated with CASODEX® (bicalutamide), LUPRON DEPOT® (leuprolide acetate for depot suspension) or FIRMAGON® (degarelix)) and a panel of normal tissues including liver, kidney, pancreas, ovary, prostate, mammary gland, colon, stomach, skeletal muscle and lung, in PBMCs obtained from healthy subjects and in prostate cancer cell lines including DU145-1, MDA-MB-436-1, LREX-1, 22RV1-1, H660-1. And other tissue cell lines including NCI-H2106-1, L-363-1, HCI-N87-1, OCI-AML5-1, MDA-PCa-2b-1 and GDM-1-1. Total RNA was extracted from formalin fixed paraffin embedded tissue samples using CELLDATA's RNAstorm-RNA isolation kit following kit protocol. RNA from cultured cell lines and PBMCs were isolated using Qiagen RNA isolation kits using standard methods. 200 ng of Total RNA from FFPE samples was used to prepare cDNA using High-capacity cDNA reverse transcription kit (ABI) and standard protocols. 37.5 ng cDNA was preamplified with gene markers in 15 μl preamplification mix using TaqMan preamplification kit (ThermoFisher Scientific) and standard protocols. To test gene expression of the identified neoantigens in the various samples, primers spanning the breakpoint sequences were designed for each of the identified prostate neoantigens and expression was assessed using Fluidigm Biomark™ HD. Percent (%) of expression positive FFPE prostate cancer samples were recorded for each neoantigen with relative average CT calculated in the prostate cancer samples. The results of the expression profiling of select neoantigens is shown in Table 21. The prevalence of each neoantigen in TCGA, SU2C and GTEx database is shown in Table 22.

TABLE 21

| | Amino | | | | |
|---|---|---|---|---|---|
| | acid | | qPCR | | |
| Neo- | SEQ | Polynucleotide | % | Relative | Normal |
| antigen | ID | SEQ | Positive | Average | Tissue |
| ID | NO: | ID NO: | FFPE | Ct | Expression |
| AS18 | 275 | 276 | 95.6 | 6.3 | Ovary, Prostate |
| P87 | 381 | 382 | 85.6 | 8.3 | Ovary, Prostate |
| AS55 | 333 | 334 | 83.3 | 8.2 | Prostate |
| AS57 | 337 | 338 | 83.3 | 7.9 | Prostate |
| AS15 | 269 | 270 | 68.9 | 11.4 | Ovary, Mammary |
| | | | | | Gland |
| AS7 | 253 | 254 | 57.8 | 11.0 | None |
| AS43 | 309 | 310 | 52.2 | 11.2 | Mammary Gland |
| AS51 | 325 | 326 | 47.8 | 10.5 | Ovary |
| AS16 | 271 | 272 | 47.8 | 10.8 | Ovary |
| AS41 | 305 | 306 | 45.6 | 11.6 | Ovary |
| AS6 | 251 | 252 | 33.3 | 10.0 | None |
| AS3 | 245 | 246 | 26.7 | 10.8 | None |
| AS11 | 261 | 262 | 25.6 | 12.1 | None |
| AS13 | 265 | 266 | 21.1 | 11.1 | None |
| AS47 | 317 | 318 | 16.7 | 12.3 | Ovary |
| AS8 | 255 | 256 | 13.3 | 12.5 | None |
| AS19 | 277 | 278 | 95.6 | 6.3 | Ovary, Prostate |
| AS37 | 297 | 298 | 0.0 | N/A | None |
| AS23 | 285 | 286 | 22.0 | 13.0 | Ovary, Prostate, |
| | | | | | Mammary Gland |
| MS1 | 437 | 448 | N/A | N/A | None |
| MS3 | 439 | 450 | N/A | N/A | None |
| MS6 | 442 | 453 | N/A | N/A | None |
| MS8 | 444 | 455 | N/A | N/A | None |
| P82 | 379 | 380 | 37.0 | 11 | |
| P16 | 343 | 344 | 76 | 9 | Prostate |
| FUS1 | 211 | 212 | 72 | 9 | Prostate |
| P22 | 349 | 350 | 70 | 9 | Prostate |
| FUS2 | 213 | 214 | 55 | 11 | Mammary Gland |
| FUS3 | 215 | 216 | 43 | 11 | Prostate |
| FUS6 | 221 | 222 | 19 | 11 | None |
| FUS5 | 219 | 220 | 14 | 7 | None |
| FUS8 | 225 | 226 | 11 | 14 | None |
| FUS15 | 345 | 346 | 8 | 13 | None |
| P35 | 353 | 354 | 5 | 13 | None |
| FUS19 | 235 | 236 | 4 | 13 | None |
| FUS7 | 223 | 224 | 0 | N/A | None |
| M84 | 167 | 168 | N/A | N/A | N/A |
| M86 | 171 | 172 | N/A | N/A | N/A |
| M10 | 19 | 20 | N/A | N/A | N/A |
| M12 | 23 | 24 | N/A | N/A | N/A |
| FR1 | 177 | 178 | N/A | N/A | N/A |

TABLE 22

| | Amino | Frozen | | | |
|---|---|---|---|---|---|
| | acid | prostate cancer | | | |
| Neoantigen | SEQ ID | tissues* | TCGA | SU2C | GTEx |
| ID | NO: | (n = 11) | % | % | % |
| AS18 | 275 | 54.5 | 12.6 | 20.9 | 0.03 |
| P87 | 381 | 0.0 | 0 | 20.9 | 0 |
| AS55 | 333 | 18.2 | 12.1 | 0 | 0 |
| AS57 | 337 | 36.4 | 14.9 | 0 | 0 |
| AS15 | 269 | 36.4 | 4.5 | 46.5 | 0 |
| AS7 | 253 | 27.3 | 5.1 | 16.3 | 0 |
| AS43 | 309 | 0.0 | 0 | 31 | 0.4 |
| AS51 | 325 | 9.1 | 0 | 23.8 | 0.52 |
| AS16 | 271 | 45.5 | 0.6 | 18.6 | 0 |
| AS41 | 305 | 0.0 | 0 | 33.3 | 0.16 |
| AS6 | 251 | 27.3 | 5.1 | 16.3 | 0 |
| AS3 | 245 | 27.3 | 10.4 | 25.6 | 0.03 |
| AS11 | 261 | 9.1 | 1.2 | 48.8 | 0.07 |
| AS13 | 265 | 27.3 | 5.7 | 32.6 | 0 |
| AS47 | 317 | 0.0 | 0 | 23.8 | 0.05 |
| AS8 | 255 | 0.0 | 0 | 14 | 0 |
| AS19 | 277 | 54.5 | 12.6 | 20.9 | 0.03 |
| AS37 | 297 | 0.0 | 0 | 38.1 | 0 |
| AS23 | 285 | 45.5 | 3.1 | 18.6 | 0.09 |
| MS1 | 437 | 18.2 | 0 | 0 | 0 |
| MS3 | 439 | 9.1 | 0.197 | 0 | 0.13 |
| MS6 | 442 | 9.1 | 0.197 | 0 | 0 |
| MS8 | 444 | 18.2 | 0 | 0 | 0.016 |
| P82 | 379 | | Varied | | |
| | | | Expression | | |
| P16 | 343 | | 27.17 | 2.33 | 0.02 |
| FUS1 | 211 | | 17.72 | 13.95 | #N/A |
| P22 | 349 | | 30.51 | 23.26 | 0.03 |
| FUS2 | 213 | | 63.58 | 46.51 | 1.78 |
| FUS3 | 215 | | 35.04 | 23.26 | 0.02 |
| FUS6 | 221 | | 21.46 | 32.56 | #N/A |
| FUS5 | 219 | | 12.40 | 18.60 | #N/A |
| FUS8 | 225 | | 1.18 | 32.56 | 0.54 |
| FUS15 | 345 | | 0.39 | 9.30 | 0.11 |
| P35 | 353 | | 1.38 | 6.98 | #N/A |
| FUS19 | 235 | | 8.86 | 30.23 | 1.04 |
| FUS7 | 223 | | 3.35 | 16.28 | 0.51 |
| M84 | 167 | | | 1.28 | |
| M86 | 171 | | | 1.09 | |
| M10 | 19 | | | 1.18 | |
| M12 | 23 | | | 0.99 | |
| FR1 | 177 | | | 0.30 | |

### Example 10 Generation of Viral Vectors Encoding the Identified Neoantigens

The identified neoantigens were validated and prioritized for their inclusion into a universal prostate cancer vaccine. 41 of the identified neoantigens were selected to be included into the expression cassettes based on their expression across prostate cancer samples, low expression in normal tissues, binding to HLA, and immunogenicity. The selected 41 neoantigens are shown in Table 21 and Table 22 and include:

AS18
(WKFEMSYTVGGPPPHVHARPRHWKTDR; SEQ ID NO: 275),
P87
(YEAGMTLGGKILFFLFLLLPLSPFSLIF; SEQ ID NO: 381),
AS55
(DGHSYTSKVNCLLLQDGFHGCVSITGAAGRRNLSIFLFLMLCKLEFHAC; SEQ ID
NO: 333),
AS57
(TGGKSTCSAPGPQSLPSTPFSTYPQWVILITEL; SEQ ID NO: 337),
AS15
(VLRFLDLKVRYLHS; SEQ ID NO: 269),
AS7
(DYWAQKEKGSSSFLRPSC; SEQ ID NO: 253),
AS43
(VPFRELKNVSVLEGLRQGRLGGPCSCHCPRPSQARLTPVDVAGPFLCLGDPGLFPPVKSS
I; SEQ ID NO: 309),
AS51
(GMECTLGQVGAPSPRREEDGWRGGHSRFKADVPAPQGPCWGGQPGSAPSSAPPEQSLL
D; SEQ ID NO: 325),
AS16
(GNTTLQQLGEASQAPSGSLIPLRLPLLWEVRG; SEQ ID NO: 271),
AS41
(EAFQRAAGEGGPGRGGARRGARVLQSPFCRAGAGEWLGHQSLR; SEQ ID NO:
305),
AS6
(DYWAQKEKISIPRTHLC (SEQ ID NO: 251),
AS3
(VAMMVPDRQVHYDFGL (SEQ ID NO: 245),
AS11
(VPFRELKNQRTAQGAPGIHHAASPVAANLCDPARHAQHTRIPCGAGQVRAGRGPEAGG
GVLQPQRPAPEKPGCPCRRGQPRLHTVKMWRA; SEQ ID NO: 261),
AS13
(KRSFAVTERII; SEQ ID NO: 265),
AS47
(FKKFDGPCGERGGGRTARALWARGDSVLTPALDPQTPVRAPSLTRAAAAV; SEQ
ID NO: 317),
AS8
(LVLGVLSGHSGSRL; SEQ ID NO: 255),
AS19
(QWQHYHRSGEAAGTPLWRPTRN; SEQ ID NO: 277),
AS37
(CHLFLQPQVGTPPPHTASARAPSGPPHPHESCPAGRRPARAAQTCARRQHGLPGCEEAG
TARVPSLHLHLHQAALGAGRGRGWGEACAQVPPSRG; SEQ ID NO: 297),
AS23
(KIQNKNCPD; SEQ ID NO: 285),
MS1
(HYKLIQQPISLFSITDRLHKTFSQLPSVHLCSITFQWGHPPIFCSTNDICVTANFCISVTFLK
PCFLLHEASASQ; SEQ ID NO: 437),
MS3
(RTALTHNQDFSIYRLCCKRGSLCHASQARSPAPPKPVRPLPAPITRITPQLGGQSDSSQPL
LTTGRPQGWQDQALRHTQQASPASCATITIPIHSAALGDHSGDPGPAWDTCPPLPLTTLIP
RAPPPYGDSTARSWPSRCGPLG; SEQ ID NO: 439),
MS6
(YAYKDFLWCFPFSLVFLQEIQICCHVSCLCCICCSTRICLGCLLELFLSRALRALHVLWNG
FQLHCQ; SEQ ID NO: 442),
MS8
(TMPAILKLQKNCLLSL; SEQ ID NO: 444),
P82
(YEAGMTLGEKFRVGNCKHLKMTRP; SEQ ID NO: 379).
P16
(GVPGDSTRRAVRRMNTF; SEQ ID NO: 343),
FUS1
(CGASACDVSLIAMDSA; SEQ ID NO: 211),
P22
(SLYHREKQLIAMDSAI; SEQ ID NO: 349),
FUS2
(TEYNQKLQVNQFSESK; SEQ ID NO: 213),
FUS3
(TEISCCTLSSEENEYLPRPEWQLQ; SEQ ID NO: 215),
FUS6
(CEERGAAGSLISCE; SEQ ID NO: 221),
FUS5
(NSKMALNSEALSVVSE; SEQ ID NO: 219),
FUS8
(WGMELAASRRFSWDHHSAGGPPRVPSVRSGAAQVQPKDPLPLRTLAGCLARTAHLRPG
AESLPQPQLHCT; SEQ ID NO: 225),
FUS15
(HVVGYGHLDTSGSSSSSSWP; SEQ ID NO: 345),
P35
(NSKMALNSLNSIDDAQLTRIAPPRSHCCFWEVNAP; SEQ ID NO: 353),
FUS19
(KMHFSLKEHPPPPCPP; SEQ ID NO: 235),
and
FUS7
(LWFQSSELSPTGAPWPSRRPTWRGTTVSPRTATSSARTCCGTKWPSSQEAALGLGSGLL
RFSCGTAAIR; SEQ ID NO: 223),
M84
(IARELHQFAFDLLIKSH; SEQ ID NO: 167),
M86
(QPDSFAALHSSLNELGE; SEQ ID NO: 171),
M10
(FVQGKDWGLKKFIRRDF; SEQ ID NO: 19),
M12
(FVQGKDWGVKKFIRRDF; SEQ ID NO: 23),
and
FR1
(QNLQNGGGSRSSATLPGRRRRRWLRRRRQPISVAPAGPPRRPNQKPNPPGGARCVIMRP
TWPGTSAFT; SEQ ID NO: 177).

Expression cassettes were designed for cloning into viral backbones Modified Vaccinia Ankara (MVA) and Great Ape Adenovirus 20 (GAd20) by joining the 41 neoantigen sequences one after the other without any linker. Each neoantigen sequences was codon-optimized for expression in either MVA or GAd20. The optimized polynucleotide sequences are shown in Table 23 for GAd20 and Table 24 for MVA expression.

TABLE 23

| | | | Codon- | |
|---|---|---|---|---|
| | | Amino | optimized | |
| | | acid | polynucleotide | |
| Neo- | | SEQ | for GAd20 | |
| antigen | | ID | expression; | Codon-optimized polynucleotide |
| ID | Gene ID | NO: | SEQ ID NO: | sequence for GAd20 expression |
| AS18 | NWD1 | 275 | 459 | TGGAAGTTCGAGATGAGCTACACCGTCGGC |
| | | | | GGACCTCCACCTCATGTTCATGCCAGACCTC |
| | | | | GGCACTGGAAAACCGACAGA |
| P87 | AR- | 381 | 460 | TATGAGGCCGGCATGACACTCGGCGGCAAG |
| | Intron | | | ATCCTGTTCTTCCTGTTCCTGCTGCTCCCTCT |
| | | | | GAGCCCCTTCAGCCTGATCTTC |
| AS55 | SPOC | 333 | 461 | GATGGCCACAGCTACACCAGCAAAGTGAAC |
| | | | | TGCCTCCTGCTGCAGGATGGCTTCCACGGCT |
| | | | | GTGTGTCTATTACTGGCGCCGCTGGCAGAC |
| | | | | GGAACCTGAGCATCTTTCTGTTTCTGATGCT |
| | | | | GTGCAAGCTCGAGTTCCACGCCTGC |
| AS57 | KLK3 | 337 | 462 | ACAGGCGGCAAGTCCACATGTTCTGCCCCT |
| | | | | GGACCTCAGAGCCTGCCTAGCACACCCTTC |
| | | | | AGCACATACCCTCAGTGGGTCATCCTGATC |
| | | | | ACCGAACTC |
| AS15 | LRRC45 | 269 | 463 | GTGCTGAGATTCCTGGACCTGAAAGTGCGC |
| | | | | TACCTGCACAGC |
| AS7 | ACSM1 | 253 | 464 | GACTATTGGGCTCAAAAAGAGAAGGGCAGC |
| | | | | AGCAGCTTCCTGCGGCCTAGCTGT |
| AS43 | CPNE7 | 309 | 465 | GTCCCCTTCAGAGAGCTGAAGAACGTTTCC |
| | | | | GTGCTGGAAGGCCTGAGACAGGGCAGACTT |
| | | | | GGCGGCCCTTGTAGCTGTCACTGCCCCAGA |
| | | | | CCTAGTCAGGCCAGACTGACACCTGTGGAT |
| | | | | GTGGCCGGACCTTTCCTGTGTCTGGGAGATC |
| | | | | CTGGCCTGTTTCCACCTGTGAAGTCCAGCATC |
| AS51 | CPNE7 | 325 | 466 | GGCATGGAATGCACCCTGGGACAAGTGGGA |
| | | | | GCCCCATCTCCTAGAAGAGAAGAGGATGGC |
| | | | | TGGCGCGGAGGCCACTCTAGATTCAAAGCT |
| | | | | GATGTGCCCGCTCCTCAGGGCCCTTGTTGGG |
| | | | | GAGGACAACCTGGATCTGCCCCATCTTCTGC |
| | | | | CCCACCTGAACAGTCCCTGCTGGAT |
| AS16 | LRRC45 | 271 | 467 | GGCAACACAACCCTCCAGCAACTGGGAGAA |
| | | | | GCCTCTCAGGCTCCTAGCGGCTCTCTGATCC |
| | | | | CTCTCAGACTGCCTCTCCTGTGGGAAGTTCG |
| | | | | GGGC |
| AS41 | RHPN1 | 305 | 468 | GAGGCTTTCCAAAGAGCTGCTGGCGAAGGC |
| | | | | GGACCTGGTAGAGGTGGTGCTAGAAGAGGT |
| | | | | GCTAGGGTGCTGCAGAGCCCATTCTGTAGA |
| | | | | GCAGGCGCAGGCGAATGGCTGGGCCATCAG |
| | | | | AGTCTGAGA |
| AS6 | ACSM1 | 251 | 469 | GATTATTGGGCCCAGAAAGAAAAGATCAGC |
| | | | | ATCCCCAGAACACACCTGTGC |
| AS3 | DNAH8 | 245 | 470 | GTGGCCATGATGGTGCCCGATAGACAGGTC |
| | | | | CACTACGACTTTGGACTG |
| AS11 | CPNE7 | 261 | 471 | GTGCCCTTCCGGGAACTGAAGAACCAGAGA |
| | | | | ACAGCTCAGGGCGCTCCTGGAATCCACCAT |
| | | | | GCTGCTTCTCCAGTGGCCGCCAACCTGTGTG |
| | | | | ATCCTGCCAGACATGCCCAGCACACCAGGA |
| | | | | TTCCTTGTGGCGCTGGACAAGTGCGCGCTG |
| | | | | GAAGAGGACCTGAAGCAGGCGGAGGTGTTC |
| | | | | TGCAACCTCAAAGACCCGCTCCTGAGAAGC |
| | | | | CTGGCTGCCCTTGCAGAAGAGGACAGCCTA |
| | | | | GACTGCACACCGTGAAAATGTGGCGAGCC |
| AS13 | GRIN3A | 265 | 472 | AAGAGAAGCTTTGCCGTGACCGAGCGGATC |
| | | | | ATC |
| AS47 | AGRN | 317 | 473 | TTCAAGAAGTTCGACGGCCCTTGCGGAGAA |
| | | | | AGAGGCGGAGGCAGAACAGCTAGAGCCCTT |
| | | | | TGGGCTAGAGGCGACAGCGTTCTGACACCA |
| | | | | GCTCTGGACCCTCAGACACCTGTTAGGGCC |
| | | | | CCTAGCCTGACAAGAGCTGCCGCCGCTGTG |
| AS8 | CACNA1 | 255 | 474 | CTGGTGCTGGGAGTGCTGTCTGGACACTCTG |
| | D | | | GCAGCAGACTG |
| AS19 | NWD1 | 277 | 475 | CAGTGGCAGCACTATCACAGATCTGGCGAA |
| | | | | GCCGCCGGAACACCCCTTTGGAGGCCAACA |
| | | | | AGAAAC |
| AS37 | RECQL4 | 297 | 476 | TGCCACTTGTTTCTGCAGCCCCAAGTGGGCA |
| | | | | CACCTCCTCCACATACAGCCTCTGCTAGAGC |
| | | | | ACCTAGCGGCCCTCCACATCCTCACGAATCT |
| | | | | TGTCCTGCCGGAAGAAGGCCTGCCAGAGCC |
| | | | | GCTCAAACATGTGCCAGACGACAGCACGGA |
| | | | | CTGCCTGGATGTGAAGAGGCTGGAACAGCC |
| | | | | AGAGTGCCTAGCCTGCACCTCCATCTGCATC |
| | | | | AGGCTGCTCTTGGAGCCGGAAGAGGTAGAG |
| | | | | GATGGGGCGAAGCTTGTGCTCAGGTGCCAC |
| | | | | CTTCTAGAGGC |
| AS23 | ZNF614 | 285 | 477 | AAGATCCAGAACAAGAACTGCCCCGAC |
| MS1 | TTLL7 | 437 | 478 | CACTACAAGCTGATCCAGCAGCCAATCAGC |
| | | | | CTGTTCAGCATCACCGACCGGCTGCACAAG |
| | | | | ACATTCAGCCAGCTGCCAAGCGTGCACCTG |
| | | | | TGCTCCATCACCTTCCAGTGGGGACACCCTC |
| | | | | CTATCTTTTGCTCCACCAACGACATCTGCGT |
| | | | | GACCGCCAACTTCTGTATCAGCGTGACCTTC |
| | | | | CTGAAGCCTTGCTTTCTGCTGCACGAGGCCA |
| | | | | GCGCCTCTCAG |
| MS3 | TESK1 | 439 | 479 | CGAACCGCTCTGACACACAACCAGGACTTC |
| | | | | AGCATCTACAGACTGTGTTGCAAGCGGGGC |
| | | | | TCCCTGTGCCATGCAAGCCAAGCTAGAAGC |
| | | | | CCCGCCTTTCCTAAACCTGTGCGACCTCTGC |
| | | | | CAGCTCCAATCACCAGAATTACCCCTCAGCT |
| | | | | CGGCGGCCAGAGCGATTCATCTCAACCTCT |
| | | | | GCTGACCACCGGCAGACCTCAAGGCTGGCA |
| | | | | AGACCAAGCTCTGAGACACACCCAGCAGGC |
| | | | | TAGCCCTGCCTCTTGTGCCACCATCACAATC |
| | | | | CCCATCCACTCTGCCGCTCTGGGCGATCATT |
| | | | | CTGGCGATCCTGGACCAGCCTGGGACACAT |
| | | | | GTCCTCCACTGCCACTCACAACACTGATCCC |
| | | | | TAGGGCTCCTCCACCTTACGGCGATTCTACC |
| | | | | GCTAGAAGCTGGCCCAGCAGATGTGGACCA |
| | | | | CTCGGA |
| MS6 | SOAT1 | 442 | 480 | TACGCCTACAAGGACTTCCTGTGGTGCTTCC |
| | | | | CCTTCTCTCTGGTGTTCCTGCAAGAGATCCA |
| | | | | GATCTGCTGTCATGTGTCCTGCCTGTGCTGC |
| | | | | ATCTGCTGTAGCACCAGAATCTGCCTGGGCT |
| | | | | GTCTGCTGGAACTGTTCCTGAGCAGAGCCCT |
| | | | | GAGAGCACTGCACGTGCTGTGGAACGGATT |
| | | | | CCAGCTGCACTGCCAG |
| MS8 | ZYG11A | 444 | 481 | ACAATGCCCGCCATCCTGAAGCTGCAGAAG |
| | | | | AATTGCCTCCTAAGCCTG |
| P82 | AR-V7 | 379 | 482 | TACGAAGCCGGGATGACCCTGGGCGAGAAG |
| | | | | TTCAGAGTGGGCAACTGCAAGCACCTGAAG |
| | | | | ATGACCCGGCCT |
| P16 | MSMB- | 343 | 483 | GGCGTGCCAGGCGATAGCACTCGGAGAGCC |
| | NCOA4-1 | | | GTCAGACGGATGAACACCTTT |
| FUS1 | SLC45A3 | 211 | 484 | TGTGGCGCCTCTGCCTGTGACGTGTCCCTGA |
| | -> ELK4 - | | | TCGCTATGGACTCCGCC |
| | 1 | | | |
| P22 | SLC45A3 - | 349 | 485 | AGCCTGTACCACCGGGAAAAGCAGCTCATT |
| | ELK4 - | | | GCCATGGACAGCGCCATC |
| | 2 | | | |
| FUS2 | ARHGEF | 213 | 486 | ACCGAGTACAACCAGAAACTGCAAGTGAAC |
| | 38-> | | | CAGTTCAGCGAGAGCAAG |
| | ARHGEF | | | |
| | 38-IT1 | | | |
| FUS3 | MSMB-> | 215 | 487 | ACCGAGATCAGCTGCTGCACCCTGAGCAGC |
| | NCOA4-2 | | | GAGGAAAACGAGTACCTGCCTAGACCTGAG |
| | | | | TGGCAGCTGCAG |
| FUS6 | TMPRSS | 221 | 488 | TGCGAAGAGAGAGGCGCCGCAGGATCTCTG |
| | 2-> ERG | | | ATCTCCTGCGAA |
| FUS5 | TMPRSS | 219 | 489 | AACAGCAAGATGGCCCTGAATAGCGAGGCC |
| | 2-> ERG | | | CTGTCTGTGGTGTCTGAA |
| FUS8 | INCA1-> | 225 | 490 | TGGGGCATGGAACTGGCCGCCAGCAGAAGA |
| | CAMTA | | | TTCAGCTGGGATCATCATAGCGCAGGCGGC |
| | 2 | | | CCACCTAGAGTGCCATCTGTTAGAAGCGGA |
| | | | | GCTGCCCAGGTGCAGCCTAAAGATCCTCTG |
| | | | | CCACTGAGAACACTGGCCGGCTGCCTTGCT |
| | | | | AGAACAGCCCATCTTAGACCTGGCGCCGAG |
| | | | | TCTCTGCCTCAGCCACAACTGCACTGTACC |
| FUS15 | D2HGD | 345 | 491 | CATGTCGTCGGCTACGGCCACCTGGATACA |
| | H-> | | | AGCGGAAGCAGCTCTAGCTCCAGCTGGCCT |
| | GAL3ST | | | |
| | 2 | | | |
| P35 | TMPRSS | 353 | 492 | AACTCAAAAATGGCTCTGAACAGCCTGAAC |
| | 2-ERG | | | TCCATCGACGACGCCCAGCTGACAAGAATC |
| | | | | GCCCCTCCTAGATCTCACTGCTGCTTTTGGG |
| | | | | AAGTGAACGCCCCA |
| FUS19 | GTF2F1-> | 235 | 493 | AAGATGCACTTTAGCCTGAAAGAACACCCT |
| | PSPN | | | CCACCACCTTGTCCTCCA |
| FUS7 | NME4-> | 223 | 494 | CTGTGGTTCCAGTCCAGCGAGCTGTCTCCTA |
| | DECR2 | | | CTGGTGCCCCTTGGCCATCTAGACGCCCTAC |
| | | | | TTGGAGAGGCACCACCGTGTCACCAAGAAC |
| | | | | CGCCACAAGCAGCGCCAGAACCTGTTGTGG |
| | | | | CACAAAGTGGCCCTCCAGCCAAGAAGCCGC |
| | | | | TCTCGGACTTGGAAGCGGACTGCTGAGGTT |
| | | | | CTCTTGTGGAACCGCCGCCATTCGG |
| M84 | AR- | 167 | 495 | ATCGCTAGAGAGCTGCACCAGTTCGCCTTC |
| | T878A | | | GACCTGCTGATCAAGAGCCAC |
| M86 | AR- | 171 | 496 | CAGCCTGATTCTTTTGCCGCACTGCACAGCT |
| | L702H | | | CCCTGAACGAGCTGGGAGAG |
| M10 | SPOP- | 19 | 497 | TTCGTGCAAGGCAAGGATTGGGGCCTCAAA |
| | F133L | | | AAGTTTATCCGCAGAGACTTC |
| M12 | SPOP- | 23 | 498 | TTTGTGCAGGGCAAAGACTGGGGCGTGAAG |
| | W133V | | | AAGTTCATCCGGCGGGACTTC |
| FR1 | ZFHX3 | 177 | 499 | CAGAACCTGCAGAACGGCGGAGGCTCTAGA |
| | | | | AGCTCTGCTACACTTCCTGGCAGGCGGCGG |
| | | | | AGAAGATGGCTGAGAAGAAGGCGGCAGCC |
| | | | | TATCTCTGTGGCTCCTGCTGGACCTCCTAGA |
| | | | | CGGCCCAACCAGAAGCCTAATCCTCCTGGC |
| | | | | GGAGCCAGATGCGTGATCATGAGGCCTACA |
| | | | | TGGCCTGGCACCAGCGCCTTCACC |

TABLE 24

| | | | Codon- | |
|---|---|---|---|---|
| | | | optimized | |
| | | Amino | polynucleotide | |
| | | acid | for MVA | |
| Neo- | | SEQ | expression; | |
| antigen | | ID | SEQ ID | Codon-optimized polynucleotide |
| ID | Gene ID | NO: | NO: | sequence for MVA expression |
| AS18 | NWD1 | 275 | 500 | TGGAAGTTCGAGATGAGCTACACCGTTGG |
| | | | | CGGCCCTCCACCACATGTTCACGCCAGAC |
| | | | | CTAGACACTGGAAAACCGACAGA |
| P87 | AR- | 381 | 501 | TACGAGGCCGGCATGACACTCGGAGGCAA |
| | Intron | | | GATCCTGTTCTTCCTGTTCCTGCTGCTCCCT |
| | | | | CTGAGCCCCTTCAGCCTGATCTTT |
| AS55 | SPOC | 333 | 461 | GATGGCCACAGCTACACCAGCAAAGTGAA |
| | | | | CTGCCTCCTGCTGCAGGATGGCTTCCACGG |
| | | | | CTGTGTGTCTATTACTGGCGCCGCTGGCAG |
| | | | | ACGGAACCTGAGCATCTTTCTGTTTCTGAT |
| | | | | GCTGTGCAAGCTCGAGTTCCACGCCTGC |
| AS57 | KLK3 | 337 | 503 | ACAGGCGGCAAGAGCACATGTTCTGCCCC |
| | | | | TGGACCTCAGTCTCTGCCCAGCACACCCTT |
| | | | | CAGCACATACCCTCAGTGGGTCATCCTGA |
| | | | | TCACCGAGCTG |
| AS15 | LRRC45 | 269 | 504 | GTGCTGCGGTTCCTGGATCTCAAAGTGCG |
| | | | | CTACCTGCACAGC |
| AS7 | ACSM1 | 253 | 505 | GATTATTGGGCCCAGAAAGAAAAGGGCAG |
| | | | | CAGCAGCTTCCTGCGGCCTAGCTGT |
| AS43 | CPNE7 | 309 | 506 | GTGCCCTTCCGGGAACTGAAGAACGTGTC |
| | | | | CGTTCTGGAAGGCCTGAGGCAGGGCAGAC |
| | | | | TTGGCGGACCTTGTAGCTGCCACTGTCCTA |
| | | | | GACCAAGCCAGGCCAGACTGACCCCTGTG |
| | | | | GATGTGGCTGGCCCATTTCTGTGTCTGGGC |
| | | | | GACCCTGGACTGTTCCCTCCAGTGAAGTCT |
| | | | | AGCATC |
| AS51 | CPNE7 | 325 | 507 | GGCATGGAATGTACACTGGGCCAAGTGGG |
| | | | | AGCCCCATCTCCTAGAAGAGAAGAGGATG |
| | | | | GCTGGCGCGGAGGCCACTCTAGATTCAAA |
| | | | | GCTGATGTGCCCGCTCCTCAGGGCCCTTGT |
| | | | | TGGGGAGGACAACCTGGATCTGCCCCATC |
| | | | | TTCTGCCCCACCTGAACAGAGCCTGCTGG |
| | | | | AT |
| AS16 | LRRC45 | 271 | 508 | GGCAACACCACACTGCAACAGCTGGGAGA |
| | | | | AGCCTCTCAGGCCCCAAGCGGTTCTCTGAT |
| | | | | CCCTCTCAGACTGCCCCTCCTGTGGGAAGT |
| | | | | GCGGGGC |
| AS41 | RHPN1 | 305 | 509 | GAGGCTTTCCAGAGAGCAGCTGGCGAAGG |
| | | | | CGGACCTGGCAGAGGTGGTGCTAGAAGAG |
| | | | | GTGCTAGAGTGCTGCAGAGCCCATTCTGT |
| | | | | AGAGCTGGCGCTGGCGAATGGCTGGGCCA |
| | | | | CCAATCTCTTAGA |
| AS6 | ACSM1 | 251 | 510 | GACTATTGGGCTCAAAAAGAGAAGATCAG |
| | | | | CATCCCCAGAACACACCTGTGC |
| AS3 | DNAH8 | 245 | 511 | GTGGCCATGATGGTGCCCGACAGACAGGT |
| | | | | GCACTACGACTTCGGCCTG |
| AS11 | CPNE7 | 261 | 512 | GTGCCCTTCAGAGAGCTGAAAAACCAGAG |
| | | | | AACAGCCCAGGGCGCTCCTGGAATCCATC |
| | | | | ATGCTGCTTCTCCAGTGGCCGCCAATCTGT |
| | | | | GCGATCCTGCCAGACATGCCCAGCATACC |
| | | | | AGGATTCCTTGTGGCGCTGGACAAGTGCG |
| | | | | CGCTGGAAGAGGACCTGAAGCTGGTGGCG |
| | | | | GAGTTCTGCAGCCTCAAAGACCTGCTCCT |
| | | | | GAGAAGCCTGGCTGCCCCTGTAGAAGAGG |
| | | | | ACAGCCTAGACTGCACACCGTGAAGATGT |
| | | | | GGCGGGCC |
| AS13 | GRIN3A | 265 | 513 | AAGAGAAGCTTCGCCGTGACCGAGCGGAT |
| | | | | CATC |
| AS47 | AGRN | 317 | 514 | TTTAAGAAGTTTGACGGCCCCTGCGGCGA |
| | | | | GAGAGGCGGAGGAAGAACTGCAAGAGCC |
| | | | | CTTTGGGCCAGAGGCGACTCTGTTCTGAC |
| | | | | ACCAGCTCTGGACCCTCAGACACCTGTTA |
| | | | | GGGCCCCTAGCCTGACAAGAGCTGCCGCT |
| | | | | GCTGTT |
| AS8 | CACNA1 | 255 | 515 | CTGGTGCTGGGCGTGCTGTCTGGCCACTCT |
| | D | | | GGAAGCAGACTG |
| AS19 | NWD1 | 277 | 516 | CAATGGCAGCACTACCACAGATCTGGCGA |
| | | | | AGCCGCTGGAACCCCACTTTGGAGGCCTA |
| | | | | CCAGAAAC |
| AS37 | RECQL4 | 297 | 517 | TGCCACTTGTTTCTCCAGCCACAAGTGGGC |
| | | | | ACCCCTCCACCTCATACAGCCTCTGCTAGA |
| | | | | GCACCTAGCGGCCCACCTCATCCTCACGA |
| | | | | ATCTTGTCCTGCCGGAAGAAGGCCTGCCA |
| | | | | GAGCCGCTCAAACATGTGCCAGACGACAG |
| | | | | CACGGACTGCCCGGATGTGAAGAAGCCGG |
| | | | | AACAGCCAGAGTGCCTAGCCTGCACCTTC |
| | | | | ATCTGCATCAGGCCGCTCTTGGAGCCGGA |
| | | | | AGAGGTAGAGGATGGGGAGAAGCTTGTGC |
| | | | | CCAGGTGCCACCTTCTAGAGGC |
| AS23 | ZNF614 | 285 | 477 | AAGATCCAGAACAAGAACTGCCCCGAC |
| MS1 | TTLL7 | 437 | 519 | CACTACAAGCTGATCCAGCAGCCAATCAG |
| | | | | CCTGTTCTCCATCACCGACCGGCTGCACAA |
| | | | | GACATTCAGCCAGCTGCCTTCCGTGCATCT |
| | | | | GTGCAGCATCACCTTCCAGTGGGGACACC |
| | | | | CTCCTATCTTTTGCTCCACCAACGACATCT |
| | | | | GCGTGACCGCCAACTTCTGTATCAGCGTG |
| | | | | ACCTTCCTGAAGCCTTGCTTTCTGCTGCAC |
| | | | | GAGGCCTCCGCCAGCCAG |
| MS3 | TESK1 | 439 | 520 | CGGACCGCTCTGACCCACAACCAGGACTT |
| | | | | CAGCATCTACCGGCTGTGCTGCAAGAGGG |
| | | | | GCTCTCTGTGTCATGCTAGCCAGGCTAGA |
| | | | | AGCCCCGCCTTTCCTAAGCCTGTCAGACCT |
| | | | | CTGCCTGCTCCTATCACCAGAATCACCCCT |
| | | | | CAGCTCGGCGGCCAGTCTGATTCATCTCA |
| | | | | GCCACTGCTGACCACCGGCAGACCTCAAG |
| | | | | GATGGCAAGACCAGGCTCTGAGACACACA |
| | | | | CAGCAGGCTAGCCCAGCCTCTTGCGCCAC |
| | | | | CATCACAATACCAATACATTCTGCCGCTCT |
| | | | | GGGCGATCACAGCGGAGATCCTGGACCTG |
| | | | | CCTGGGATACTTGTCCTCCTCTGCCCCTAA |
| | | | | CTACACTGATCCCTAGGGCTCCTCCACCTT |
| | | | | ACGGCGATAGCACAGCCAGATCCTGGCCT |
| | | | | AGCAGATGTGGCCCTCTGGGC |
| MS6 | SOAT1 | 442 | 521 | TACGCCTACAAGGACTTCCTGTGGTGCTTC |
| | | | | CCCTTCTCTCTGGTGTTCCTGCAAGAAATC |
| | | | | CAGATCTGCTGTCACGTGTCCTGCCTGTGC |
| | | | | TGTATCTGCTGTAGCACCCGGATCTGTCTG |
| | | | | GGCTGTCTGCTGGAACTGTTCCTGAGCAG |
| | | | | AGCCCTGAGAGCACTGCACGTGCTGTGGA |
| | | | | ACGGATTCCAGCTGCACTGCCAG |
| MS8 | ZYG11A | 444 | 522 | ACCATGCCTGCCATTCTGAAGCTGCAGAA |
| | | | | GAATTGTCTTCTAAGCCTG |
| P82 | AR-V7 | 379 | 523 | TATGAGGCTGGAATGACCCTGGGCGAGAA |
| | | | | GTTCAGAGTGGGCAACTGCAAGCACCTGA |
| | | | | AGATGACCCGGCCT |
| P16 | MSMB- | 343 | 524 | GGAGTGCCTGGCGATTCTACTAGAAGGGC |
| | NCOA4-1 | | | CGTGCGGCGGATGAACACCTTT |
| FUS1 | SLC45A3-> | 211 | 525 | TGTGGCGCATCTGCCTGCGACGTGTCCCTG |
| | ELK4 - 1 | | | ATCGCTATGGATAGCGCC |
| P22 | SLC45A3- | 349 | 485 | AGCCTGTACCACCGGGAAAAGCAGCTCAT |
| | ELK4 - 2 | | | TGCCATGGACAGCGCCATC |
| FUS2 | ARHGEF3 | 213 | 486 | ACCGAGTACAACCAGAAACTGCAAGTGAA |
| | 8-> | | | CCAGTTCAGCGAGAGCAAG |
| | ARHGEF3 | | | |
| | 8-IT1 | | | |
| FUS3 | MSMB-> | 215 | 528 | ACCGAGATCAGCTGCTGCACCCTGAGCAG |
| | NCOA4-2 | | | CGAGGAAAACGAGTACCTGCCTAGACCTG |
| | | | | AATGGCAGCTGCAG |
| FUS6 | TMPRSS2 | 221 | 529 | TGCGAGGAAAGAGGCGCAGCCGGATCTCT |
| | ->ERG | | | GATCTCTTGCGAG |
| FUS5 | TMPRSS2 | 219 | 530 | AACAGCAAGATGGCCCTGAATAGCGAGGC |
| | ->ERG | | | CCTGTCTGTGGTGTCCGAG |
| FUS8 | INCA1-> | 225 | 531 | TGGGGAATGGAACTGGCCGCTAGCAGGCG |
| | CAMTA2 | | | GTTTAGCTGGGATCATCATTCTGCCGGCGG |
| | | | | ACCTCCAAGAGTGCCAAGCGTTAGAAGCG |
| | | | | GAGCAGCCCAGGTCCAGCCTAAAGATCCA |
| | | | | CTGCCACTGAGAACACTGGCCGGCTGCCT |
| | | | | TGCCAGAACAGCTCATCTTAGACCTGGCG |
| | | | | CCGAAAGCCTGCCTCAACCTCAGCTGCAT |
| | | | | TGCACA |
| FUS15 | D2HGDH-> | 345 | 532 | CACGTTGTCGGCTATGGCCACCTGGATAC |
| | GAL3ST2 | | | AAGCGGCTCCTCTAGCAGTAGCTCCTGGC |
| | | | | CT |
| P35 | TMPRSS2- | 353 | 533 | AATTCTAAGATGGCTCTCAACAGCCTGAA |
| | ERG | | | CTCCATCGACGACGCCCAGCTGACAAGAA |
| | | | | TCGCCCCTCCAAGAAGCCACTGTTGCTTTT |
| | | | | GGGAAGTGAACGCCCCT |
| FUS19 | GTF2F1-> | 235 | 534 | AAGATGCACTTCTCACTGAAAGAGCACCC |
| | PSPN | | | GCCACCGCCGTGCCCACCG |
| FUS7 | NME4-> | 223 | 535 | CTGTGGTTCCAGTCCAGCGAACTGTCTCCT |
| | DECR2 | | | ACTGGCGCTCCATGGCCAAGCAGAAGGCC |
| | | | | TACTTGGAGAGGCACCACCGTGTCTCCAA |
| | | | | GAACCGCTACAAGCAGCGCCAGAACCTGT |
| | | | | TGCGGCACAAAATGGCCCTCCAGCCAAGA |
| | | | | AGCTGCCCTCGGACTTGGAAGCGGACTGC |
| | | | | TGAGATTCAGCTGTGGCACAGCCGCCATC |
| | | | | AGA |
| M84 | AR-T878A | 167 | 536 | ATCGCCAGAGAACTGCACCAGTTCGCCTT |
| | | | | CGACCTGCTGATCAAGAGCCAC |
| M86 | AR-L702H | 171 | 537 | CAGCCTGACAGCTTTGCTGCCCTGCATAGC |
| | | | | TCCCTGAATGAGCTGGGCGAA |
| M10 | SPOP- | 19 | 538 | TTTGTGCAGGGTAAAGATTGGGGCCTCAA |
| | F133L | | | AAAGTTTATCAGACGGGACTTC |
| M12 | SPOP- | 23 | 539 | TTCGTGCAGGGCAAAGACTGGGGCGTGAA |
| | W133V | | | GAAGTTCATCCGGCGGGACTTT |
| FR1 | ZFHX3 | 177 | 540 | CAGAACCTGCAGAACGGCGGAGGCTCTAG |
| | | | | AAGCTCTGCTACACTTCCTGGCAGGCGGC |
| | | | | GGAGAAGATGGCTGAGAAGAAGGCGGCA |
| | | | | GCCTATCTCTGTGGCTCCTGCTGGACCTCC |
| | | | | TAGACGGCCCAACCAGAAGCCTAATCCTC |
| | | | | CTGGCGGAGCCAGATGCGTGATCATGAGG |
| | | | | CCTACATGGCCTGGCACCAGCGCCTTTACC |

### Synthetic Gene Design

The 41 neoantigen amino acidic sequences were joined head to tail. The order of the neoantigens sequences was determined according to a strategy that minimized the formation of predicted junctional epitopes that may be generated by the juxtaposition of two adjacent neoantigen peptides.

To this purpose, custom tools were developed to split the 41 neoantigens into 4 smaller lists (sublists) of similar cumulative length and to generate, for each sublist, 2 million scrambled layouts of the synthetic gene with a different neoantigen order. The tool proceeded iteratively. At each loop a scrambled layout was generated and compared to the layouts already generated. If the number of predicted junctional epitopes in the new layout was lower than the number of the previously best layout, the new layout was considered as the best. Each scrambled layout was analyzed estimating the number of potential junctional epitopes predicted to bind one out of a subset of 9 class I HLA haplotypes with an IC₅₀<=1500 nM (considering only 9mer epitopes predicted by the IEDB_recommended method included in the IEDB 2.17 software). The 9 class I HLA haplotypes cumulatively cover 82% of the world population as estimated by analyzing haplotypes annotated for subjects in the 1000 genomes project. Scrambled layouts with neoantigens that formed predicted junctional epitopes with the N-terminal T-cell enhancer or the C-terminal TAG sequence were excluded. As an additional constraint, in each layout junctions that contained a 9mer peptide that matched a protein annotated in the human wildtype proteome were also excluded.

The best layouts obtained after scrambling 2 million times each of the 4 sublists were then joined to generate an overall layout comprising all 41 neoantigens. Out of all possible combinations of the best 4 layouts the one with the minimal number of predicted epitopes formed by the newly formed junctions was selected.

The whole procedure described was applied two times independently to generate two artificial genes to be encoded alternatively by the GAd20 or MVA vector. For the MVA vector the scrambled layouts were designed with the additional constraint of avoiding the junctions with predicted junctional epitopes that were already present in the layout selected for the Adenoviral transgene.

Amino acid sequences of the optimized layout for the GAd20 is shown in SEQ ID NO: 541 and for MVA SEQ ID NO: 543. Neoantigens in the GAd20 insert of SEQ ID NO: 541 were in the following order: FR1-AS13-AS7-AS6-AS8-P87-FUS3-AS43-AS57-AS51-AS18-AS55-AS23-AS47-MS1-AS37-AS15-AS19-AS11-AS3-P16-P82-FUS5-FUS1-M12-MS6-FUS2-P22-FUS6-MS8-MS3-AS16-M86-M84-M10-FUS8-FUS7-FUS19-AS41-FUS15-P35. Neoantigens in the MVA insert of SEQ DID NO: 543 were in the following order: FR1-AS51-AS6-AS18-AS7-AS43-FUS3-P87-AS8-AS13-AS57-AS55-AS19-AS3-AS23-AS15-AS11-AS37-MS1-AS47-P16-FUS1-FUS6-P22-M12-MS8-FUS5-P82-FUS2-MS3-MS6-AS16-P35-M10-AS41-FUS8-M84-FUS19-FUS15-M86-FUS7.

Five additional alternative optimized layouts of scrambled neoantigens were assessed for each vector. The five alternative layouts had the same number of predicted junctional epitopes compared to SEQ ID NO: 541 and SEQ ID NO: 543. The five alternative layouts for Gad20 are shown in SEQ ID NO: 554, SEQ ID NO; 555, SEQ ID NO: 556, SEQ ID NO: 623 and SEQ ID NO: 624. The five alternative layouts for MVA are shown in SEQ ID NO: 557, SEQ ID NO: 558, SEQ ID NO: 559, SEQ ID NO: 625 and SEQ ID NO: 626. The neoantigens in the alternative optimized layouts were in the following order:

SEQ ID NO: 554: FR1-AS13-AS8-P87-FUS3-AS43-AS57-
AS51-AS7-AS6-AS18-P16-P82-FUS5-FUS1-M12-MS6-FUS2-
P22-FUS6-MS8-MS3-AS55-AS23-AS47-MS1-AS37-AS15-
AS19-AS11-AS3-AS16-M86-M84-M10-FUS8-FUS7-FUS19-
AS41-FUS15-P35
SEQ ID NO: 555: FR1-AS13-FUS3-P87-
AS7-AS43-AS57-AS51-AS6-AS8-AS18-AS55-AS23-AS47-
MS1-AS37-AS15-AS19-AS11-AS3-P16-P82-FUS5-FUS1-M12-
MS6-FUS2-P22-FUS6-MS8-MS3-AS16-M86-M84-M10-FUS8-
FUS7-FUS19-AS41-FUS15-P35
SEQ ID NO: 556: FR1-AS13-AS7-AS43-AS8-P87-FUS3-
AS57-AS51-AS6-AS18-AS55-AS23-AS47-MS1-AS37-AS15-
AS19-AS11-AS3-P16-P82-FUS5-FUS1-M12-MS6-FUS2-P22-
FUS6-MS8-MS3-AS16-M86-M84-M10-FUS8-FUS7-FUS19-
AS41-FUS15-P35
SEQ ID NO: 623: P16-P82-FUS5-FUS1-M12-MS6-FUS2-
P22-FUS6-MS8-MS3-AS16-M86-M84-M10-FUS8-FUS7-FUS19-
AS41-FUS15-P35-AS55-AS23-AS47-MS1-AS37-AS15-AS19-
AS11-AS3-FR1-AS13-AS8-P87-FUS3-AS43-AS57-AS51-AS7-
AS6-AS18
SEQ ID NO: 624: AS16-M86-M84-M10-FUS8-FUS7-FUS19-
AS41-FUS15-P35-P16-P82-FUS5-FUS1-M12-MS6-FUS2-P22-
FUS6-MS8-MS3-AS55-AS23-AS47-MS1-AS37-AS15-AS19-
AS11-AS3-FR1-AS13-FUS3-P87-AS7-AS43-AS57-AS51-AS6-
AS8-AS18
SEQ ID NO: 557: FR1-AS51-AS6-AS18-AS7-AS43-FUS3-
P87-AS8-AS13-AS57-AS55-AS37-MS1-AS3-AS23-AS15-
AS11-AS19-AS47-P16-FUS1-FUS6-P22-M12-MS8-FUS5-P82-
FUS2-MS3-MS6-AS16-P35-M10-AS41-FUS8-M84-FUS19-
FUS15-M86-FUS7
SEQ ID NO: 558: AS55-AS19-AS3-AS15-AS23-AS11-AS37-
MS1-AS47-FR1-AS51-AS6-AS18-AS7-AS43-FUS3-P87-AS8-
AS13-AS57-P16-FUS1-FUS6-P22-M12-MS8-FUS5-P82-FUS2-
MS3-MS6-AS16-P35-M10-AS41-FUS8-M84-FUS19-FUS15-
M86-FUS7
SEQ ID NO: 559: AS16-P35-M10-AS41-FUS8-M84-FUS19-
FUS15-M86-FUS7-AS55-AS19-AS3-AS23-AS15-AS11-AS37-
MS1-AS47-P16-FUS1-FUS2-P82-MS8-FUS5-FUS6-P22-M12-
MS3-MS6-FR1-AS51-AS6-AS18-AS7-AS43-FUS3-P87-AS8-
AS13-AS57
SEQ ID NO: 625: AS16-P35-M10-AS41-FUS8-M84-FUS19-
FUS15-M86-FUS7-FR1-AS51-AS6-AS18-AS7-AS43-FUS3-
P87-AS8-AS13-AS57-AS55-AS19-AS3-AS15-AS23-AS11-
AS37-MS1-AS47-P16-FUS1-FUS6-P22-M12-MS8-FUS5-P82-
FUS2-MS3-MS6
SEQ ID NO: 626: AS55-AS11-AS19-AS23-AS3-AS15-AS37-
MS1-AS47-FR1-AS51-AS6-AS18-AS7-AS43-FUS3-P87-AS8-
AS13-AS57-AS16-P35-M10-AS41-FUS8-M84-FUS19-FUS15-
M86-FUS7-P16-FUS1-FUS6-P22-M12-P82-MS8-FUS5-FUS2-
MS3-MS6

### Insertion of T-Cell Enhancer and TAG Sequences

A small peptide fragment with length of 28aa from the mandarin fish invariant chain (MGQKEQIHTLQKNSERMSKQLTRSSQAV; SEQ ID NO: 549) was placed at the N-terminus of each transgene encoding the 41 neoantigens. Preclinical data has shown this sequence to increase the immunological response of the viral vector. A small segment of 7 amino acids (TAG sequence; seq: SHHHHHH; SEQ ID NO: 627) was added at the C-terminus of the transgene for the purpose of monitoring the expression of the encoded transgene.

Amino acid sequences of the optimized layout for the GAd20 that includes the TCE sequence and omits the tag sequence are shown in SEQ ID NO: 550 and for MVA SEQ ID NO: 551.

Conversion into Nucleotide Sequence and Optimization to Remove Predicted miRNA Binding Sites.

The conversion from amino acid sequence into nucleotide sequence was performed using codon optimizing according to the human codon usage applying additional constraints to avoid as much as possible the following features:
internal TATA-boxes, chi-sites and ribosomal entry sites
AT-rich or GC-rich sequence stretches
RNA instability motifs
repeat sequences and RNA secondary structures
(cryptic) splice donor and acceptor sites in higher eukaryotes
TTTTTnT termination motifs for the MVA vector

EcoR1, BamH1 restriction sites and a KOZAK sequence were then added upstream the optimized nucleotide sequence. 2 STOP codons followed by Asc1 and Not1 restriction sites were added downstream the optimized nucleotide sequence.

The optimized nucleotide sequence of each transgene was then further analyzed with the PITA and miranda software to detect predicted miRNA target sites that might downregulate the expression of the synthetic transgene. 9 miRNA binding sites detected by both methods were removed by modifying the nucleotide sequence of the regions that are predicted to bind the miRNA “seed” by introducing synonymous changes in the corresponding codons. The synthesis of GAd20 and MVA transgenes, was performed using standard methods.

The codon optimized polynucleotide sequence encoding the GAd20 neoantigen layout of SEQ ID NO: 541 is shown in SEQ ID NO: 542.

The codon optimized polynucleotide sequence encoding the MVA (neoMVA) neoantigen layout of SEQ ID NO: 543 is shown in SEQ ID NO: 544.

The codon optimized polynucleotide sequence encoding the GAd20 neoantigen layout including the TCE sequence and excluding the TAG sequence of SEQ ID NO: 550 is shown in SEQ ID NO: 551.

The codon optimized polynucleotide sequence encoding the MVA neoantigen layout including the TCE sequence and excluding the TAG sequence of SEQ ID NO: 552 is shown in SEQ ID NO: 553.

Kozak sequence: CGCGACTTCGCCGCC; SEQ ID NO: 545

Polynucleotide encoding the TCE:

SEQ ID NO: 546
ATGGGCCAGAAAGAGCAGATCCACACACTGCAGAAAAACAGCGAGCGGAT
GAGCAAGCAGCTGACCAGATCTTCTCAGGCCGTG;

Polynucleotide encoding the serine-histidine tag: AGCCATCACCATCACCACCAT; SEQ ID NO: 547

Two stop codons (TAGTAA)

Polypeptide sequence of the TCE: MGQKEQIHTLQKNSERMSKQLTRSSQAV; SEQ ID NO: 549

### GAd20 Virus Production

The GAd20 transgene was subcloned into a shuttle plasmid between CMV promoter with two TetO repeats and a BGH polyA via ECOR1-NOT1 restriction sites.

The resulting expression cassette was transferred into the GAd20 genome by homologous recombination in suitable E. coli strains, transformed with the CMV-transgene-BGH DNA fragment and with a construct carrying the GAd20 genome.

Recombination involved CMV and BGH as homology arms, that were already present in the GAd20 construct in place of the E1 deletion (insertion site of the transgene). Recombinant GAd20 vectors were then rescued by transfection of the E1 complementing, TetR expressing M9 cells and amplified by subsequent re-infection of fresh M9 cells.

CMV promoter with TetO sites:
SEQ ID NO: 628
Ccattgcatacgttgtatccatatcataatatgtacatttatattggctca
tgtccaacattaccgccatgttgacattgattattgactagttattaatag
taatcaattacggggtcattagttcatagcccatatatggagttccgcgtt
acataacttacggtaaatggcccgcctggctgaccgcccaacgacccccgc
ccattgacgtcaataatgacgtatgttcccatagtaacgccaatagggact
ttccattgacgtcaatgggtggagtatttacggtaaactgcccacttggca
gtacatcaagtgtatcatatgccaagtacgccccctattgacgtcaatgac
ggtaaatggcccgcctggcattatgcccagtacatgaccttatgggacttt
cctacttggcagtacatctacgtattagtcatcgctattaccatggtgatg
cggttttggcagtacatcaatgggcgtggatagcggtttgactcacgggga
tttccaagtctccaccccattgacgtcaatgggagtttgttttggcaccaa
aatcaacgggactttccaaaatgtcgtaacaactccgccccattgacgcaa
atgggcggtaggcgtgtacggtgggaggtctatataagcagagctctccct
atcagtgatagagatctccctatcagtgatagagatcgtcgacgagctcgt
ttagtgaaccgtcagatcgcctggagacgccatccacgctgttttgacctc
catagaagacaccgggaccgatccagcctccgcggccgggaacggtgcatt
ggaacgcggattccccgtgccaagagtga
BGH polyA
SEQ ID NO: 629
ctgtgccttctagttgccagccatctgttgtttgcccctcccccgtgcctt
ccttgaccctggaaggtgccactcccactgtcctttcctaataaaatgagg
aaattgcatcgcattgtctgagtaggtgtcattctattctggggggtgggg
tggggcaggacagcaagggggaggattgggaagacaatagcaggcatgctg
gggatgcggtgggctctatggcc

### MVA Virus Production

The MVA transgene was subcloned into the p94 shuttle plasmid via BAMH1-ASC1 restriction sites, under the control of the vaccinia P7.5 early/late promoter (SEQ ID NO: 630), between sequences homologous to the deletion III locus of MVA (FlankIII-1 and -2 regions). An additional expression cassette for eGFP protein, flanked by a repeated sequence named “Z”, was present in the p94 shuttle plasmid, between Flank III regions.

The parental MVA vector used for recombinant vaccine viruses' generation carried the HcRed1-1 fluorescent protein transgene at the Deletion III locus and was indicated as MVA-RED 476 MG.

Recombinant MVA, with transgene insertion at the Deletion III locus, were generated by two events of in vivo recombination in Chicken embryo fibroblasts (CEF) cells. The first recombination event occurred in cells infected with MVA-RED 476 MG and transfected with the p94 shuttle plasmid, and resulted in replacement of the HcRed protein gene with the transgene/eGFP cassette. Infected cells containing MVA-Green intermediate were isolated by FACS sorting of green cells. The intermediate recombinant MVA resulting from first recombination carried both the transgene and the eGFP cassette but was unstable due to the presence of repeated Z regions. Thus, a spontaneous second recombination event occurred involving Z regions and removed the eGFP cassette. The resulting recombinant MVA was colorless and carried the transgene cassette at the Deletion III locus (insertion site) of MVA-RED 476 MG. This was isolated by FACS sorting of colorless infected cells and amplified by re-infection of fresh CEF cells. The obtained lysate was used to infect Agel cells to produce the research batch.

P7.5 early/late promoter
SEQ ID NO: 630
GATCACTAATTCCAAACCCACCCGCTTTTTATAGTAAGTTTTTCACCCATAAATAATAAATAC
AATAATTAATTTCTCGTAAAAGTAGAAAATATATTCTAATTTATTGCACGGTAAGGAAGTAG
AATCATAAAGAACAGTGACGGATC
neoGAd20 protein (no TCE, no HIS tag)
SEQ ID NO: 541
QNLQNGGGSRSSATLPGRRRRRWLRRRRQPISVAPAGPPRRPNQKPNPPGGARCVIMRPTWPGT
SAFTKRSFAVTERIIDYWAQKEKGSSSFLRPSCDYWAQKEKISIPRTHLCLVLGVLSGHSGSRLYE
AGMTLGGKILFFLFLLLPLSPFSLIFTEISCCTLSSEENEYLPRPEWQLQVPFRELKNVSVLEGLRQ
GRLGGPCSCHCPRPSQARLTPVDVAGPFLCLGDPGLFPPVKSSITGGKSTCSAPGPQSLPSTPFSTY
PQWVILITELGMECTLGQVGAPSPRREEDGWRGGHSRFKADVPAPQGPCWGGQPGSAPSSAPPE
QSLLDWKFEMSYTVGGPPPHVHARPRHWKTDRDGHSYTSKVNCLLLQDGFHGCVSITGAAGRR
NLSIFLFLMLCKLEFHACKIQNKNCPDFKKFDGPCGERGGGRTARALWARGDSVLTPALDPQTP
VRAPSLTRAAAAVHYKLIQQPISLFSITDRLHKTFSQLPSVHLCSITFQWGHPPIFCSTNDICVTANF
CISVTFLKPCFLLHEASASQCHLFLQPQVGTPPPHTASARAPSGPPHPHESCPAGRRPARAAQTCA
RRQHGLPGCEEAGTARVPSLHLHLHQAALGAGRGRGWGEACAQVPPSRGVLRFLDLKVRYLHS
QWQHYHRSGEAAGTPLWRPTRNVPFRELKNQRTAQGAPGIHHAASPVAANLCDPARHAQHTRI
PCGAGQVRAGRGPEAGGGVLQPQRPAPEKPGCPCRRGQPRLHTVKMWRAVAMMVPDRQVHY
DFGLGVPGDSTRRAVRRMNTFYEAGMTLGEKFRVGNCKHLKMTRPNSKMALNSEALSVVSEC
GASACDVSLIAMDSAFVQGKDWGVKKFIRRDFYAYKDFLWCFPFSLVFLQEIQICCHVSCLCCIC
CSTRICLGCLLELFLSRALRALHVLWNGFQLHCQTEYNQKLQVNQFSESKSLYHREKQLIAMDS
AICEERGAAGSLISCETMPAILKLQKNCLLSLRTALTHNQDFSIYRLCCKRGSLCHASQARSPAFP
KPVRPLPAPITRITPQLGGQSDSSQPLLTTGRPQGWQDQALRHTQQASPASCATITIPIHSAALGDH
SGDPGPAWDTCPPLPLTTLIPRAPPPYGDSTARSWPSRCGPLGGNTTLQQLGEASQAPSGSLIPLR
LPLLWEVRGQPDSFAALHSSLNELGEIARELHQFAFDLLIKSHFVQGKDWGLKKFIRRDFWGME
LAASRRFSWDHHSAGGPPRVPSVRSGAAQVQPKDPLPLRTLAGCLARTAHLRPGAESLPQPQLH
CTLWFQSSELSPTGAPWPSRRPTWRGTTVSPRTATSSARTCCGTKWPSSQEAALGLGSGLLRFSC
GTAAIRKMHFSLKEHPPPPCPPEAFQRAAGEGGPGRGGARRGARVLQSPFCRAGAGEWLGHQSL
RHVVGYGHLDTSGSSSSSSWPNSKMALNSLNSIDDAQLTRIAPPRSHCCFWEVNAP
neoGAd20 polynucleotide (no TCE, no HIS tag)
SEQ ID NO: 542
CAGAACCTGCAGAACGGCGGAGGCTCTAGAAGCTCTGCTACACTTCCTGGCAGGCGGCGGA
GAAGATGGCTGAGAAGAAGGCGGCAGCCTATCTCTGTGGCTCCTGCTGGACCTCCTAGACG
GCCCAACCAGAAGCCTAATCCTCCTGGCGGAGCCAGATGCGTGATCATGAGGCCTACATGG
CCTGGCACCAGCGCCTTCACCAAGAGAAGCTTTGCCGTGACCGAGCGGATCATCGACTATTG
GGCTCAAAAAGAGAAGGGCAGCAGCAGCTTCCTGCGGCCTAGCTGTGATTATTGGGCCCAG
AAAGAAAAGATCAGCATCCCCAGAACACACCTGTGCCTGGTGCTGGGAGTGCTGTCTGGAC
ACTCTGGCAGCAGACTGTATGAGGCCGGCATGACACTCGGCGGCAAGATCCTGTTCTTCCTG
TTCCTGCTGCTCCCTCTGAGCCCCTTCAGCCTGATCTTCACCGAGATCAGCTGCTGCACCCTG
AGCAGCGAGGAAAACGAGTACCTGCCTAGACCTGAGTGGCAGCTGCAGGTCCCCTTCAGAG
AGCTGAAGAACGTTTCCGTGCTGGAAGGCCTGAGACAGGGCAGACTTGGCGGCCCTTGTAG
CTGTCACTGCCCCAGACCTAGTCAGGCCAGACTGACACCTGTGGATGTGGCCGGACCTTTCC
TGTGTCTGGGAGATCCTGGCCTGTTTCCACCTGTGAAGTCCAGCATCACAGGCGGCAAGTCC
ACATGTTCTGCCCCTGGACCTCAGAGCCTGCCTAGCACACCCTTCAGCACATACCCTCAGTG
GGTCATCCTGATCACCGAACTCGGCATGGAATGCACCCTGGGACAAGTGGGAGCCCCATCTC
CTAGAAGAGAAGAGGATGGCTGGCGCGGAGGCCACTCTAGATTCAAAGCTGATGTGCCCGC
TCCTCAGGGCCCTTGTTGGGGAGGACAACCTGGATCTGCCCCATCTTCTGCCCCACCTGAAC
AGTCCCTGCTGGATTGGAAGTTCGAGATGAGCTACACCGTCGGCGGACCTCCACCTCATGTT
CATGCCAGACCTCGGCACTGGAAAACCGACAGAGATGGCCACAGCTACACCAGCAAAGTGA
ACTGCCTCCTGCTGCAGGATGGCTTCCACGGCTGTGTGTCTATTACTGGCGCCGCTGGCAGA
CGGAACCTGAGCATCTTTCTGTTTCTGATGCTGTGCAAGCTCGAGTTCCACGCCTGCAAGATC
CAGAACAAGAACTGCCCCGACTTCAAGAAGTTCGACGGCCCTTGCGGAGAAAGAGGCGGAG
GCAGAACAGCTAGAGCCCTTTGGGCTAGAGGCGACAGCGTTCTGACACCAGCTCTGGACCCT
CAGACACCTGTTAGGGCCCCTAGCCTGACAAGAGCTGCCGCCGCTGTGCACTACAAGCTGAT
CCAGCAGCCAATCAGCCTGTTCAGCATCACCGACCGGCTGCACAAGACATTCAGCCAGCTGC
CAAGCGTGCACCTGTGCTCCATCACCTTCCAGTGGGGACACCCTCCTATCTTTTGCTCCACCA
ACGACATCTGCGTGACCGCCAACTTCTGTATCAGCGTGACCTTCCTGAAGCCTTGCTTTCTGC
TGCACGAGGCCAGCGCCTCTCAGTGCCACTTGTTTCTGCAGCCCCAAGTGGGCACACCTCCT
CCACATACAGCCTCTGCTAGAGCACCTAGCGGCCCTCCACATCCTCACGAATCTTGTCCTGC
CGGAAGAAGGCCTGCCAGAGCCGCTCAAACATGTGCCAGACGACAGCACGGACTGCCTGGA
TGTGAAGAGGCTGGAACAGCCAGAGTGCCTAGCCTGCACCTCCATCTGCATCAGGCTGCTCT
TGGAGCCGGAAGAGGTAGAGGATGGGGCGAAGCTTGTGCTCAGGTGCCACCTTCTAGAGGC
GTGCTGAGATTCCTGGACCTGAAAGTGCGCTACCTGCACAGCCAGTGGCAGCACTATCACAG
ATCTGGCGAAGCCGCCGGAACACCCCTTTGGAGGCCAACAAGAAACGTGCCCTTCCGGGAA
CTGAAGAACCAGAGAACAGCTCAGGGCGCTCCTGGAATCCACCATGCTGCTTCTCCAGTGGC
CGCCAACCTGTGTGATCCTGCCAGACATGCCCAGCACACCAGGATTCCTTGTGGCGCTGGAC
AAGTGCGCGCTGGAAGAGGACCTGAAGCAGGCGGAGGTGTTCTGCAACCTCAAAGACCCGC
TCCTGAGAAGCCTGGCTGCCCTTGCAGAAGAGGACAGCCTAGACTGCACACCGTGAAAATG
TGGCGAGCCGTGGCCATGATGGTGCCCGATAGACAGGTCCACTACGACTTTGGACTGGGCGT
GCCAGGCGATAGCACTCGGAGAGCCGTCAGACGGATGAACACCTTTTACGAAGCCGGGATG
ACCCTGGGCGAGAAGTTCAGAGTGGGCAACTGCAAGCACCTGAAGATGACCCGGCCTAACA
GCAAGATGGCCCTGAATAGCGAGGCCCTGTCTGTGGTGTCTGAATGTGGCGCCTCTGCCTGT
GACGTGTCCCTGATCGCTATGGACTCCGCCTTTGTGCAGGGCAAAGACTGGGGCGTGAAGAA
GTTCATCCGGCGGGACTTCTACGCCTACAAGGACTTCCTGTGGTGCTTCCCCTTCTCTCTGGT
GTTCCTGCAAGAGATCCAGATCTGCTGTCATGTGTCCTGCCTGTGCTGCATCTGCTGTAGCAC
CAGAATCTGCCTGGGCTGTCTGCTGGAACTGTTCCTGAGCAGAGCCCTGAGAGCACTGCACG
TGCTGTGGAACGGATTCCAGCTGCACTGCCAGACCGAGTACAACCAGAAACTGCAAGTGAA
CCAGTTCAGCGAGAGCAAGAGCCTGTACCACCGGGAAAAGCAGCTCATTGCCATGGACAGC
GCCATCTGCGAAGAGAGAGGCGCCGCAGGATCTCTGATCTCCTGCGAAACAATGCCCGCCA
TCCTGAAGCTGCAGAAGAATTGCCTCCTAAGCCTGCGAACCGCTCTGACACACAACCAGGAC
TTCAGCATCTACAGACTGTGTTGCAAGCGGGGCTCCCTGTGCCATGCAAGCCAAGCTAGAAG
CCCCGCCTTTCCTAAACCTGTGCGACCTCTGCCAGCTCCAATCACCAGAATTACCCCTCAGCT
CGGCGGCCAGAGCGATTCATCTCAACCTCTGCTGACCACCGGCAGACCTCAAGGCTGGCAA
GACCAAGCTCTGAGACACACCCAGCAGGCTAGCCCTGCCTCTTGTGCCACCATCACAATCCC
CATCCACTCTGCCGCTCTGGGCGATCATTCTGGCGATCCTGGACCAGCCTGGGACACATGTC
CTCCACTGCCACTCACAACACTGATCCCTAGGGCTCCTCCACCTTACGGCGATTCTACCGCTA
GAAGCTGGCCCAGCAGATGTGGACCACTCGGAGGCAACACAACCCTCCAGCAACTGGGAGA
AGCCTCTCAGGCTCCTAGCGGCTCTCTGATCCCTCTCAGACTGCCTCTCCTGTGGGAAGTTCG
GGGCCAGCCTGATTCTTTTGCCGCACTGCACAGCTCCCTGAACGAGCTGGGAGAGATCGCTA
GAGAGCTGCACCAGTTCGCCTTCGACCTGCTGATCAAGAGCCACTTCGTGCAAGGCAAGGAT
TGGGGCCTCAAAAAGTTTATCCGCAGAGACTTCTGGGGCATGGAACTGGCCGCCAGCAGAA
GATTCAGCTGGGATCATCATAGCGCAGGCGGCCCACCTAGAGTGCCATCTGTTAGAAGCGG
AGCTGCCCAGGTGCAGCCTAAAGATCCTCTGCCACTGAGAACACTGGCCGGCTGCCTTGCTA
GAACAGCCCATCTTAGACCTGGCGCCGAGTCTCTGCCTCAGCCACAACTGCACTGTACCCTG
TGGTTCCAGTCCAGCGAGCTGTCTCCTACTGGTGCCCCTTGGCCATCTAGACGCCCTACTTGG
AGAGGCACCACCGTGTCACCAAGAACCGCCACAAGCAGCGCCAGAACCTGTTGTGGCACAA
AGTGGCCCTCCAGCCAAGAAGCCGCTCTCGGACTTGGAAGCGGACTGCTGAGGTTCTCTTGT
GGAACCGCCGCCATTCGGAAGATGCACTTTAGCCTGAAAGAACACCCTCCACCACCTTGTCC
TCCAGAGGCTTTCCAAAGAGCTGCTGGCGAAGGCGGACCTGGTAGAGGTGGTGCTAGAAGA
GGTGCTAGGGTGCTGCAGAGCCCATTCTGTAGAGCAGGCGCAGGCGAATGGCTGGGCCATC
AGAGTCTGAGACATGTCGTCGGCTACGGCCACCTGGATACAAGCGGAAGCAGCTCTAGCTC
CAGCTGGCCTAACTCAAAAATGGCTCTGAACAGCCTGAACTCCATCGACGACGCCCAGCTGA
CAAGAATCGCCCCTCCTAGATCTCACTGCTGCTTTTGGGAAGTGAACGCCCCA
neoMVA protein (no TCE, no HIS Tag)
SEQ ID NO: 543
QNLQNGGGSRSSATLPGRRRRRWLRRRRQPISVAPAGPPRRPNQKPNPPGGARCVIMRPTWPGT
SAFTGMECTLGQVGAPSPRREEDGWRGGHSRFKADVPAPQGPCWGGQPGSAPSSAPPEQSLLDD
YWAQKEKISIPRTHLCWKFEMSYTVGGPPPHVHARPRHWKTDRDYWAQKEKGSSSFLRPSCVP
FRELKNVSVLEGLRQGRLGGPCSCHCPRPSQARLTPVDVAGPFLCLGDPGLFPPVKSSITEISCCTL
SSEENEYLPRPEWQLQYEAGMTLGGKILFFLFLLLPLSPFSLIFLVLGVLSGHSGSRLKRSFAVTER
IITGGKSTCSAPGPQSLPSTPFSTYPQWVILITELDGHSYTSKVNCLLLQDGFHGCVSITGAAGRRN
LSIFLFLMLCKLEFHACQWQHYHRSGEAAGTPLWRPTRNVAMMVPDRQVHYDFGLKIQNKNCP
DVLRFLDLKVRYLHSVPFRELKNQRTAQGAPGIHHAASPVAANLCDPARHAQHTRIPCGAGQVR
AGRGPEAGGGVLQPQRPAPEKPGCPCRRGQPRLHTVKMWRACHLFLQPQVGTPPPHTASARAP
SGPPHPHESCPAGRRPARAAQTCARRQHGLPGCEEAGTARVPSLHLHLHQAALGAGRGRGWGE
ACAQVPPSRGHYKLIQQPISLFSITDRLHKTFSQLPSVHLCSITFQWGHPPIFCSTNDICVTANFCIS
VTFLKPCFLLHEASASQFKKFDGPCGERGGGRTARALWARGDSVLTPALDPQTPVRAPSLTRAA
AAVGVPGDSTRRAVRRMNTFCGASACDVSLIAMDSACEERGAAGSLISCESLYHREKQLIAMDS
AIFVQGKDWGVKKFIRRDFTMPAILKLQKNCLLSLNSKMALNSEALSVVSEYEAGMTLGEKFRV
GNCKHLKMTRPTEYNQKLQVNQFSESKRTALTHNQDFSIYRLCCKRGSLCHASQARSPAFPKPV
RPLPAPITRITPQLGGQSDSSQPLLTTGRPQGWQDQALRHTQQASPASCATITIPIHSAALGDHSGD
PGPAWDTCPPLPLTTLIPRAPPPYGDSTARSWPSRCGPLGYAYKDFLWCFPFSLVFLQEIQICCHV
SCLCCICCSTRICLGCLLELFLSRALRALHVLWNGFQLHCQGNTTLQQLGEASQAPSGSLIPLRLP
LLWEVRGNSKMALNSLNSIDDAQLTRIAPPRSHCCFWEVNAPFVQGKDWGLKKFIRRDFEAFQR
AAGEGGPGRGGARRGARVLQSPFCRAGAGEWLGHQSLRWGMELAASRRFSWDHHSAGGPPRV
PSVRSGAAQVQPKDPLPLRTLAGCLARTAHLRPGAESLPQPQLHCTIARELHQFAFDLLIKSHKM
HFSLKEHPPPPCPPHVVGYGHLDTSGSSSSSSWPQPDSFAALHSSLNELGELWFQSSELSPTGAPW
PSRRPTWRGTTVSPRTATSSARTCCGTKWPSSQEAALGLGSGLLRFSCGTAAIR
neoMVA polynucleotide
SEQ ID NO: 544
CAGAACCTGCAGAACGGCGGAGGCTCTAGAAGCTCTGCTACACTTCCTGGCAGGCGGCGGA
GAAGATGGCTGAGAAGAAGGCGGCAGCCTATCTCTGTGGCTCCTGCTGGACCTCCTAGACG
GCCCAACCAGAAGCCTAATCCTCCTGGCGGAGCCAGATGCGTGATCATGAGGCCTACATGG
CCTGGCACCAGCGCCTTTACCGGCATGGAATGTACACTGGGCCAAGTGGGAGCCCCATCTCC
TAGAAGAGAAGAGGATGGCTGGCGCGGAGGCCACTCTAGATTCAAAGCTGATGTGCCCGCT
CCTCAGGGCCCTTGTTGGGGAGGACAACCTGGATCTGCCCCATCTTCTGCCCCACCTGAACA
GAGCCTGCTGGATGACTATTGGGCTCAAAAAGAGAAGATCAGCATCCCCAGAACACACCTG
TGCTGGAAGTTCGAGATGAGCTACACCGTTGGCGGCCCTCCACCACATGTTCACGCCAGACC
TAGACACTGGAAAACCGACAGAGATTATTGGGCCCAGAAAGAAAAGGGCAGCAGCAGCTTC
CTGCGGCCTAGCTGTGTGCCCTTCCGGGAACTGAAGAACGTGTCCGTTCTGGAAGGCCTGAG
GCAGGGCAGACTTGGCGGACCTTGTAGCTGCCACTGTCCTAGACCAAGCCAGGCCAGACTG
ACCCCTGTGGATGTGGCTGGCCCATTTCTGTGTCTGGGCGACCCTGGACTGTTCCCTCCAGTG
AAGTCTAGCATCACCGAGATCAGCTGCTGCACCCTGAGCAGCGAGGAAAACGAGTACCTGC
CTAGACCTGAATGGCAGCTGCAGTACGAGGCCGGCATGACACTCGGAGGCAAGATCCTGTT
CTTCCTGTTCCTGCTGCTCCCTCTGAGCCCCTTCAGCCTGATCTTTCTGGTGCTGGGCGTGCTG
TCTGGCCACTCTGGAAGCAGACTGAAGAGAAGCTTCGCCGTGACCGAGCGGATCATCACAG
GCGGCAAGAGCACATGTTCTGCCCCTGGACCTCAGTCTCTGCCCAGCACACCCTTCAGCACA
TACCCTCAGTGGGTCATCCTGATCACCGAGCTGGATGGCCACAGCTACACCAGCAAAGTGAA
CTGCCTCCTGCTGCAGGATGGCTTCCACGGCTGTGTGTCTATTACTGGCGCCGCTGGCAGAC
GGAACCTGAGCATCTTTCTGTTTCTGATGCTGTGCAAGCTCGAGTTCCACGCCTGCCAATGGC
AGCACTACCACAGATCTGGCGAAGCCGCTGGAACCCCACTTTGGAGGCCTACCAGAAACGT
GGCCATGATGGTGCCCGACAGACAGGTGCACTACGACTTCGGCCTGAAGATCCAGAACAAG
AACTGCCCCGACGTGCTGCGGTTCCTGGATCTCAAAGTGCGCTACCTGCACAGCGTGCCCTT
CAGAGAGCTGAAAAACCAGAGAACAGCCCAGGGCGCTCCTGGAATCCATCATGCTGCTTCT
CCAGTGGCCGCCAATCTGTGCGATCCTGCCAGACATGCCCAGCATACCAGGATTCCTTGTGG
CGCTGGACAAGTGCGCGCTGGAAGAGGACCTGAAGCTGGTGGCGGAGTTCTGCAGCCTCAA
AGACCTGCTCCTGAGAAGCCTGGCTGCCCCTGTAGAAGAGGACAGCCTAGACTGCACACCG
TGAAGATGTGGCGGGCCTGCCACTTGTTTCTCCAGCCACAAGTGGGCACCCCTCCACCTCAT
ACAGCCTCTGCTAGAGCACCTAGCGGCCCACCTCATCCTCACGAATCTTGTCCTGCCGGAAG
AAGGCCTGCCAGAGCCGCTCAAACATGTGCCAGACGACAGCACGGACTGCCCGGATGTGAA
GAAGCCGGAACAGCCAGAGTGCCTAGCCTGCACCTTCATCTGCATCAGGCCGCTCTTGGAGC
CGGAAGAGGTAGAGGATGGGGAGAAGCTTGTGCCCAGGTGCCACCTTCTAGAGGCCACTAC
AAGCTGATCCAGCAGCCAATCAGCCTGTTCTCCATCACCGACCGGCTGCACAAGACATTCAG
CCAGCTGCCTTCCGTGCATCTGTGCAGCATCACCTTCCAGTGGGGACACCCTCCTATCTTTTG
CTCCACCAACGACATCTGCGTGACCGCCAACTTCTGTATCAGCGTGACCTTCCTGAAGCCTT
GCTTTCTGCTGCACGAGGCCTCCGCCAGCCAGTTTAAGAAGTTTGACGGCCCCTGCGGCGAG
AGAGGCGGAGGAAGAACTGCAAGAGCCCTTTGGGCCAGAGGCGACTCTGTTCTGACACCAG
CTCTGGACCCTCAGACACCTGTTAGGGCCCCTAGCCTGACAAGAGCTGCCGCTGCTGTTGGA
GTGCCTGGCGATTCTACTAGAAGGGCCGTGCGGCGGATGAACACCTTTTGTGGCGCATCTGC
CTGCGACGTGTCCCTGATCGCTATGGATAGCGCCTGCGAGGAAAGAGGCGCAGCCGGATCT
CTGATCTCTTGCGAGAGCCTGTACCACCGGGAAAAGCAGCTCATTGCCATGGACAGCGCCAT
CTTCGTGCAGGGCAAAGACTGGGGCGTGAAGAAGTTCATCCGGCGGGACTTTACCATGCCTG
CCATTCTGAAGCTGCAGAAGAATTGTCTTCTAAGCCTGAACAGCAAGATGGCCCTGAATAGC
GAGGCCCTGTCTGTGGTGTCCGAGTATGAGGCTGGAATGACCCTGGGCGAGAAGTTCAGAG
TGGGCAACTGCAAGCACCTGAAGATGACCCGGCCTACCGAGTACAACCAGAAACTGCAAGT
GAACCAGTTCAGCGAGAGCAAGCGGACCGCTCTGACCCACAACCAGGACTTCAGCATCTAC
CGGCTGTGCTGCAAGAGGGGCTCTCTGTGTCATGCTAGCCAGGCTAGAAGCCCCGCCTTTCC
TAAGCCTGTCAGACCTCTGCCTGCTCCTATCACCAGAATCACCCCTCAGCTCGGCGGCCAGT
CTGATTCATCTCAGCCACTGCTGACCACCGGCAGACCTCAAGGATGGCAAGACCAGGCTCTG
AGACACACACAGCAGGCTAGCCCAGCCTCTTGCGCCACCATCACAATACCAATACATTCTGC
CGCTCTGGGCGATCACAGCGGAGATCCTGGACCTGCCTGGGATACTTGTCCTCCTCTGCCCC
TAACTACACTGATCCCTAGGGCTCCTCCACCTTACGGCGATAGCACAGCCAGATCCTGGCCT
AGCAGATGTGGCCCTCTGGGCTACGCCTACAAGGACTTCCTGTGGTGCTTCCCCTTCTCTCTG
GTGTTCCTGCAAGAAATCCAGATCTGCTGTCACGTGTCCTGCCTGTGCTGTATCTGCTGTAGC
ACCCGGATCTGTCTGGGCTGTCTGCTGGAACTGTTCCTGAGCAGAGCCCTGAGAGCACTGCA
CGTGCTGTGGAACGGATTCCAGCTGCACTGCCAGGGCAACACCACACTGCAACAGCTGGGA
GAAGCCTCTCAGGCCCCAAGCGGTTCTCTGATCCCTCTCAGACTGCCCCTCCTGTGGGAAGT
GCGGGGCAATTCTAAGATGGCTCTCAACAGCCTGAACTCCATCGACGACGCCCAGCTGACA
AGAATCGCCCCTCCAAGAAGCCACTGTTGCTTTTGGGAAGTGAACGCCCCTTTTGTGCAGGG
TAAAGATTGGGGCCTCAAAAAGTTTATCAGACGGGACTTCGAGGCTTTCCAGAGAGCAGCT
GGCGAAGGCGGACCTGGCAGAGGTGGTGCTAGAAGAGGTGCTAGAGTGCTGCAGAGCCCAT
TCTGTAGAGCTGGCGCTGGCGAATGGCTGGGCCACCAATCTCTTAGATGGGGAATGGAACTG
GCCGCTAGCAGGCGGTTTAGCTGGGATCATCATTCTGCCGGCGGACCTCCAAGAGTGCCAAG
CGTTAGAAGCGGAGCAGCCCAGGTCCAGCCTAAAGATCCACTGCCACTGAGAACACTGGCC
GGCTGCCTTGCCAGAACAGCTCATCTTAGACCTGGCGCCGAAAGCCTGCCTCAACCTCAGCT
GCATTGCACAATCGCCAGAGAACTGCACCAGTTCGCCTTCGACCTGCTGATCAAGAGCCACA
AGATGCACTTCTCACTGAAAGAGCACCCGCCACCGCCGTGCCCACCGCACGTTGTCGGCTAT
GGCCACCTGGATACAAGCGGCTCCTCTAGCAGTAGCTCCTGGCCTCAGCCTGACAGCTTTGC
TGCCCTGCATAGCTCCCTGAATGAGCTGGGCGAACTGTGGTTCCAGTCCAGCGAACTGTCTC
CTACTGGCGCTCCATGGCCAAGCAGAAGGCCTACTTGGAGAGGCACCACCGTGTCTCCAAG
AACCGCTACAAGCAGCGCCAGAACCTGTTGCGGCACAAAATGGCCCTCCAGCCAAGAAGCT
GCCCTCGGACTTGGAAGCGGACTGCTGAGATTCAGCTGTGGCACAGCCGCCATCAGA
neoGAd20 expression cassette protein
SEQ ID NO: 550
MGQKEQIHTLQKNSERMSKQLTRSSQAVQNLQNGGGSRSSATLPGRRRRRWLRRRRQPISVAPA
GPPRRPNQKPNPPGGARCVIMRPTWPGTSAFTKRSFAVTERIIDYWAQKEKGSSSFLRPSCDYWA
QKEKISIPRTHLCLVLGVLSGHSGSRLYEAGMTLGGKILFFLFLLLPLSPFSLIFTEISCCTLSSEENE
YLPRPEWQLQVPFRELKNVSVLEGLRQGRLGGPCSCHCPRPSQARLTPVDVAGPFLCLGDPGLFP
PVKSSITGGKSTCSAPGPQSLPSTPFSTYPQWVILITELGMECTLGQVGAPSPRREEDGWRGGHSR
FKADVPAPQGPCWGGQPGSAPSSAPPEQSLLDWKFEMSYTVGGPPPHVHARPRHWKTDRDGHS
YTSKVNCLLLQDGFHGCVSITGAAGRRNLSIFLFLMLCKLEFHACKIQNKNCPDFKKFDGPCGER
GGGRTARALWARGDSVLTPALDPQTPVRAPSLTRAAAAVHYKLIQQPISLFSITDRLHKTFSQLPS
VHLCSITFQWGHPPIFCSTNDICVTANFCISVTFLKPCFLLHEASASQCHLFLQPQVGTPPPHTASA
RAPSGPPHPHESCPAGRRPARAAQTCARRQHGLPGCEEAGTARVPSLHLHLHQAALGAGRGRG
WGEACAQVPPSRGVLRFLDLKVRYLHSQWQHYHRSGEAAGTPLWRPTRNVPFRELKNQRTAQ
GAPGIHHAASPVAANLCDPARHAQHTRIPCGAGQVRAGRGPEAGGGVLQPQRPAPEKPGCPCRR
GQPRLHTVKMWRAVAMMVPDRQVHYDFGLGVPGDSTRRAVRRMNTFYEAGMTLGEKFRVGN
CKHLKMTRPNSKMALNSEALSVVSECGASACDVSLIAMDSAFVQGKDWGVKKFIRRDFYAYKD
FLWCFPFSLVFLQEIQICCHVSCLCCICCSTRICLGCLLELFLSRALRALHVLWNGFQLHCQTEYN
QKLQVNQFSESKSLYHREKQLIAMDSAICEERGAAGSLISCETMPAILKLQKNCLLSLRTALTHN
QDFSIYRLCCKRGSLCHASQARSPAFPKPVRPLPAPITRITPQLGGQSDSSQPLLTTGRPQGWQDQ
ALRHTQQASPASCATITIPIHSAALGDHSGDPGPAWDTCPPLPLTTLIPRAPPPYGDSTARSWPSRC
GPLGGNTTLQQLGEASQAPSGSLIPLRLPLLWEVRGQPDSFAALHSSLNELGEIARELHQFAFDLL
IKSHFVQGKDWGLKKFIRRDFWGMELAASRRFSWDHHSAGGPPRVPSVRSGAAQVQPKDPLPL
RTLAGCLARTAHLRPGAESLPQPQLHCTLWFQSSELSPTGAPWPSRRPTWRGTTVSPRTATSSAR
TCCGTKWPSSQEAALGLGSGLLRFSCGTAAIRKMHFSLKEHPPPPCPPEAFQRAAGEGGPGRGGA
RRGARVLQSPFCRAGAGEWLGHQSLRHVVGYGHLDTSGSSSSSSWPNSKMALNSLNSIDDAQLT
RIAPPRSHCCFWEVNAP
neoGAd20 expression cassette polynucleotide
SEQ ID NO: 551
Ccattgcatacgttgtatccatatcataatatgtacatttatattggctcatgtccaacattaccgccatgttgacattgattattgactagttattaatag
taatcaattacggggtcattagttcatagcccatatatggagttccgcgttacataacttacggtaaatggcccgcctggctgaccgcccaacgacccccgc
ccattgacgtcaataatgacgtatgttcccatagtaacgccaatagggactttccattgacgtcaatgggtggagtatttacggtaaactgcccacttggca
gtacatcaagtgtatcatatgccaagtacgccccctattgacgtcaatgacggtaaatggcccgcctggcattatgcccagtacatgaccttatgggacttt
cctacttggcagtacatctacgtattagtcatcgctattaccatggtgatgcggttttggcagtacatcaatgggcgtggatagcggtttgactcacgggga
tttccaagtctccaccccattgacgtcaatgggagtagttaggcaccaaaatcaacgggactaccaaaatgtcgtaacaactccgccccattgacgcaaatg
ggcggtaggcgtgtacggtgggaggtctatataagcagagctctccctatcagtgatagagatctccctatcagtgatagagatcgtcgacgagctcgttta
cgtgaacgtcagatcgcctggagacgccatccacgctgttttgacctccatagaagacaccgggaccgatccagcctccgcggccgggaacggtgcattgga
acgcggattccccgtgccaagagtgagatcttccgtttatctaggtaccagatatcaGAATTCGGATCCCGCGACTTCGCCGCCA
TGGGCCAGAAAGAGCAGATCCACACACTGCAGAAAAACAGCGAGCGGATGAGCAAGCAGC
TGACCAGATCTTCTCAGGCCGTGCAGAACCTGCAGAACGGCGGAGGCTCTAGAAGCTCTGCT
ACACTTCCTGGCAGGCGGCGGAGAAGATGGCTGAGAAGAAGGCGGCAGCCTATCTCTGTGG
CTCCTGCTGGACCTCCTAGACGGCCCAACCAGAAGCCTAATCCTCCTGGCGGAGCCAGATGC
GTGATCATGAGGCCTACATGGCCTGGCACCAGCGCCTTCACCAAGAGAAGCTTTGCCGTGAC
CGAGCGGATCATCGACTATTGGGCTCAAAAAGAGAAGGGCAGCAGCAGCTTCCTGCGGCCT
AGCTGTGATTATTGGGCCCAGAAAGAAAAGATCAGCATCCCCAGAACACACCTGTGCCTGG
TGCTGGGAGTGCTGTCTGGACACTCTGGCAGCAGACTGTATGAGGCCGGCATGACACTCGGC
GGCAAGATCCTGTTCTTCCTGTTCCTGCTGCTCCCTCTGAGCCCCTTCAGCCTGATCTTCACC
GAGATCAGCTGCTGCACCCTGAGCAGCGAGGAAAACGAGTACCTGCCTAGACCTGAGTGGC
AGCTGCAGGTCCCCTTCAGAGAGCTGAAGAACGTTTCCGTGCTGGAAGGCCTGAGACAGGG
CAGACTTGGCGGCCCTTGTAGCTGTCACTGCCCCAGACCTAGTCAGGCCAGACTGACACCTG
TGGATGTGGCCGGACCTTTCCTGTGTCTGGGAGATCCTGGCCTGTTTCCACCTGTGAAGTCCA
GCATCACAGGCGGCAAGTCCACATGTTCTGCCCCTGGACCTCAGAGCCTGCCTAGCACACCC
TTCAGCACATACCCTCAGTGGGTCATCCTGATCACCGAACTCGGCATGGAATGCACCCTGGG
ACAAGTGGGAGCCCCATCTCCTAGAAGAGAAGAGGATGGCTGGCGCGGAGGCCACTCTAGA
TTCAAAGCTGATGTGCCCGCTCCTCAGGGCCCTTGTTGGGGAGGACAACCTGGATCTGCCCC
ATCTTCTGCCCCACCTGAACAGTCCCTGCTGGATTGGAAGTTCGAGATGAGCTACACCGTCG
GCGGACCTCCACCTCATGTTCATGCCAGACCTCGGCACTGGAAAACCGACAGAGATGGCCA
CAGCTACACCAGCAAAGTGAACTGCCTCCTGCTGCAGGATGGCTTCCACGGCTGTGTGTCTA
TTACTGGCGCCGCTGGCAGACGGAACCTGAGCATCTTTCTGTTTCTGATGCTGTGCAAGCTC
GAGTTCCACGCCTGCAAGATCCAGAACAAGAACTGCCCCGACTTCAAGAAGTTCGACGGCC
CTTGCGGAGAAAGAGGCGGAGGCAGAACAGCTAGAGCCCTTTGGGCTAGAGGCGACAGCGT
TCTGACACCAGCTCTGGACCCTCAGACACCTGTTAGGGCCCCTAGCCTGACAAGAGCTGCCG
CCGCTGTGCACTACAAGCTGATCCAGCAGCCAATCAGCCTGTTCAGCATCACCGACCGGCTG
CACAAGACATTCAGCCAGCTGCCAAGCGTGCACCTGTGCTCCATCACCTTCCAGTGGGGACA
CCCTCCTATCTTTTGCTCCACCAACGACATCTGCGTGACCGCCAACTTCTGTATCAGCGTGAC
CTTCCTGAAGCCTTGCTTTCTGCTGCACGAGGCCAGCGCCTCTCAGTGCCACTTGTTTCTGCA
GCCCCAAGTGGGCACACCTCCTCCACATACAGCCTCTGCTAGAGCACCTAGCGGCCCTCCAC
ATCCTCACGAATCTTGTCCTGCCGGAAGAAGGCCTGCCAGAGCCGCTCAAACATGTGCCAGA
CGACAGCACGGACTGCCTGGATGTGAAGAGGCTGGAACAGCCAGAGTGCCTAGCCTGCACC
TCCATCTGCATCAGGCTGCTCTTGGAGCCGGAAGAGGTAGAGGATGGGGCGAAGCTTGTGCT
CAGGTGCCACCTTCTAGAGGCGTGCTGAGATTCCTGGACCTGAAAGTGCGCTACCTGCACAG
CCAGTGGCAGCACTATCACAGATCTGGCGAAGCCGCCGGAACACCCCTTTGGAGGCCAACA
AGAAACGTGCCCTTCCGGGAACTGAAGAACCAGAGAACAGCTCAGGGCGCTCCTGGAATCC
ACCATGCTGCTTCTCCAGTGGCCGCCAACCTGTGTGATCCTGCCAGACATGCCCAGCACACC
AGGATTCCTTGTGGCGCTGGACAAGTGCGCGCTGGAAGAGGACCTGAAGCAGGCGGAGGTG
TTCTGCAACCTCAAAGACCCGCTCCTGAGAAGCCTGGCTGCCCTTGCAGAAGAGGACAGCCT
AGACTGCACACCGTGAAAATGTGGCGAGCCGTGGCCATGATGGTGCCCGATAGACAGGTCC
ACTACGACTTTGGACTGGGCGTGCCAGGCGATAGCACTCGGAGAGCCGTCAGACGGATGAA
CACCTTTTACGAAGCCGGGATGACCCTGGGCGAGAAGTTCAGAGTGGGCAACTGCAAGCAC
CTGAAGATGACCCGGCCTAACAGCAAGATGGCCCTGAATAGCGAGGCCCTGTCTGTGGTGTC
TGAATGTGGCGCCTCTGCCTGTGACGTGTCCCTGATCGCTATGGACTCCGCCTTTGTGCAGGG
CAAAGACTGGGGCGTGAAGAAGTTCATCCGGCGGGACTTCTACGCCTACAAGGACTTCCTGT
GGTGCTTCCCCTTCTCTCTGGTGTTCCTGCAAGAGATCCAGATCTGCTGTCATGTGTCCTGCC
TGTGCTGCATCTGCTGTAGCACCAGAATCTGCCTGGGCTGTCTGCTGGAACTGTTCCTGAGC
AGAGCCCTGAGAGCACTGCACGTGCTGTGGAACGGATTCCAGCTGCACTGCCAGACCGAGT
ACAACCAGAAACTGCAAGTGAACCAGTTCAGCGAGAGCAAGAGCCTGTACCACCGGGAAAA
GCAGCTCATTGCCATGGACAGCGCCATCTGCGAAGAGAGAGGCGCCGCAGGATCTCTGATC
TCCTGCGAAACAATGCCCGCCATCCTGAAGCTGCAGAAGAATTGCCTCCTAAGCCTGCGAAC
CGCTCTGACACACAACCAGGACTTCAGCATCTACAGACTGTGTTGCAAGCGGGGCTCCCTGT
GCCATGCAAGCCAAGCTAGAAGCCCCGCCTTTCCTAAACCTGTGCGACCTCTGCCAGCTCCA
ATCACCAGAATTACCCCTCAGCTCGGCGGCCAGAGCGATTCATCTCAACCTCTGCTGACCAC
CGGCAGACCTCAAGGCTGGCAAGACCAAGCTCTGAGACACACCCAGCAGGCTAGCCCTGCC
TCTTGTGCCACCATCACAATCCCCATCCACTCTGCCGCTCTGGGCGATCATTCTGGCGATCCT
GGACCAGCCTGGGACACATGTCCTCCACTGCCACTCACAACACTGATCCCTAGGGCTCCTCC
ACCTTACGGCGATTCTACCGCTAGAAGCTGGCCCAGCAGATGTGGACCACTCGGAGGCAAC
ACAACCCTCCAGCAACTGGGAGAAGCCTCTCAGGCTCCTAGCGGCTCTCTGATCCCTCTCAG
ACTGCCTCTCCTGTGGGAAGTTCGGGGCCAGCCTGATTCTTTTGCCGCACTGCACAGCTCCCT
GAACGAGCTGGGAGAGATCGCTAGAGAGCTGCACCAGTTCGCCTTCGACCTGCTGATCAAG
AGCCACTTCGTGCAAGGCAAGGATTGGGGCCTCAAAAAGTTTATCCGCAGAGACTTCTGGG
GCATGGAACTGGCCGCCAGCAGAAGATTCAGCTGGGATCATCATAGCGCAGGCGGCCCACC
TAGAGTGCCATCTGTTAGAAGCGGAGCTGCCCAGGTGCAGCCTAAAGATCCTCTGCCACTGA
GAACACTGGCCGGCTGCCTTGCTAGAACAGCCCATCTTAGACCTGGCGCCGAGTCTCTGCCT
CAGCCACAACTGCACTGTACCCTGTGGTTCCAGTCCAGCGAGCTGTCTCCTACTGGTGCCCCT
TGGCCATCTAGACGCCCTACTTGGAGAGGCACCACCGTGTCACCAAGAACCGCCACAAGCA
GCGCCAGAACCTGTTGTGGCACAAAGTGGCCCTCCAGCCAAGAAGCCGCTCTCGGACTTGG
AAGCGGACTGCTGAGGTTCTCTTGTGGAACCGCCGCCATTCGGAAGATGCACTTTAGCCTGA
AAGAACACCCTCCACCACCTTGTCCTCCAGAGGCTTTCCAAAGAGCTGCTGGCGAAGGCGGA
CCTGGTAGAGGTGGTGCTAGAAGAGGTGCTAGGGTGCTGCAGAGCCCATTCTGTAGAGCAG
GCGCAGGCGAATGGCTGGGCCATCAGAGTCTGAGACATGTCGTCGGCTACGGCCACCTGGA
TACAAGCGGAAGCAGCTCTAGCTCCAGCTGGCCTAACTCAAAAATGGCTCTGAACAGCCTG
AACTCCATCGACGACGCCCAGCTGACAAGAATCGCCCCTCCTAGATCTCACTGCTGCTTTTG
GGAAGTGAACGCCCCAAGCCATCACCATCACCACCATTAGTAAAGGCGCGCCTAGCGGCCG
Cgatctgctgtgccttctagttgccagccatctgttgtttgcccctcccccgtgccttccttgaccctggaaggtgccactcccactgtcctttcctaataa
aatgaggaaattgcatcgcattgtctgagtaggtgtcattctattctggggggtggggtggggcaggacagcaagggggaggattgggaagacaatagcag
gcatgctggggatgcggtgggctctatggcc
neoMVA expression cassette protein
SEQ ID NO: 552
MGQKEQIHTLQKNSERMSKQLTRSSQAVQNLQNGGGSRSSATLPGRRRRRWLRRRRQPISVAPA
GPPRRPNQKPNPPGGARCVIMRPTWPGTSAFTGMECTLGQVGAPSPRREEDGWRGGHSRFKAD
VPAPQGPCWGGQPGSAPSSAPPEQSLLDDYWAQKEKISIPRTHLCWKFEMSYTVGGPPPHVHAR
PRHWKTDRDYWAQKEKGSSSFLRPSCVPFRELKNVSVLEGLRQGRLGGPCSCHCPRPSQARLTP
VDVAGPFLCLGDPGLFPPVKSSITEISCCTLSSEENEYLPRPEWQLQYEAGMTLGGKILFFLFLLLP
LSPFSLIFLVLGVLSGHSGSRLKRSFAVTERIITGGKSTCSAPGPQSLPSTPFSTYPQWVILITELDGH
SYTSKVNCLLLQDGFHGCVSITGAAGRRNLSIFLFLMLCKLEFHACQWQHYHRSGEAAGTPLWR
PTRNVAMMVPDRQVHYDFGLKIQNKNCPDVLRFLDLKVRYLHSVPFRELKNQRTAQGAPGIHH
AASPVAANLCDPARHAQHTRIPCGAGQVRAGRGPEAGGGVLQPQRPAPEKPGCPCRRGQPRLH
TVKMWRACHLFLQPQVGTPPPHTASARAPSGPPHPHESCPAGRRPARAAQTCARRQHGLPGCEE
AGTARVPSLHLHLHQAALGAGRGRGWGEACAQVPPSRGHYKLIQQPISLFSITDRLHKTFSQLPS
VHLCSITFQWGHPPIFCSTNDICVTANFCISVTFLKPCFLLHEASASQFKKFDGPCGERGGGRTAR
ALWARGDSVLTPALDPQTPVRAPSLTRAAAAVGVPGDSTRRAVRRMNTFCGASACDVSLIAMD
SACEERGAAGSLISCESLYHREKQLIAMDSAIFVQGKDWGVKKFIRRDFTMPAILKLQKNCLLSL
NSKMALNSEALSVVSEYEAGMTLGEKFRVGNCKHLKMTRPTEYNQKLQVNQFSESKRTALTHN
QDFSIYRLCCKRGSLCHASQARSPAFPKPVRPLPAPITRITPQLGGQSDSSQPLLTTGRPQGWQDQ
ALRHTQQASPASCATITIPIHSAALGDHSGDPGPAWDTCPPLPLTTLIPRAPPPYGDSTARSWPSRC
GPLGYAYKDFLWCFPFSLVFLQEIQICCHVSCLCCICCSTRICLGCLLELFLSRALRALHVLWNGF
QLHCQGNTTLQQLGEASQAPSGSLIPLRLPLLWEVRGNSKMALNSLNSIDDAQLTRIAPPRSHCC
FWEVNAPFVQGKDWGLKKFIRRDFEAFQRAAGEGGPGRGGARRGARVLQSPFCRAGAGEWLG
HQSLRWGMELAASRRFSWDHHSAGGPPRVPSVRSGAAQVQPKDPLPLRTLAGCLARTAHLRPG
AESLPQPQLHCTIARELHQFAFDLLIKSHKMHFSLKEHPPPPCPPHVVGYGHLDTSGSSSSSSWPQ
PDSFAALHSSLNELGELWFQSSELSPTGAPWPSRRPTWRGTTVSPRTATSSARTCCGTKWPSSQE
AALGLGSGLLRFSCGTAAIR
neoMVA expression cassette polynucleotide
SEQ ID NO: 553
gatcactaattccaaacccacccgctttttatagtaagtttttcacccataaataataaatacaataattaatttctcgtaaaagtagaaaatatattctaa
tttattgcacggtaaggaagtagaatcataaagaacagtgacGGATCCCGCGACTTCGCCGCCATGGGCCAGAAAGAGCAG
ATCCACACACTGCAGAAAAACAGCGAGCGGATGAGCAAGCAGCTGACCAGATCTTCTCAGG
CCGTGCAGAACCTGCAGAACGGCGGAGGCTCTAGAAGCTCTGCTACACTTCCTGGCAGGCG
GCGGAGAAGATGGCTGAGAAGAAGGCGGCAGCCTATCTCTGTGGCTCCTGCTGGACCTCCT
AGACGGCCCAACCAGAAGCCTAATCCTCCTGGCGGAGCCAGATGCGTGATCATGAGGCCTA
CATGGCCTGGCACCAGCGCCTTTACCGGCATGGAATGTACACTGGGCCAAGTGGGAGCCCC
ATCTCCTAGAAGAGAAGAGGATGGCTGGCGCGGAGGCCACTCTAGATTCAAAGCTGATGTG
CCCGCTCCTCAGGGCCCTTGTTGGGGAGGACAACCTGGATCTGCCCCATCTTCTGCCCCACCT
GAACAGAGCCTGCTGGATGACTATTGGGCTCAAAAAGAGAAGATCAGCATCCCCAGAACAC
ACCTGTGCTGGAAGTTCGAGATGAGCTACACCGTTGGCGGCCCTCCACCACATGTTCACGCC
AGACCTAGACACTGGAAAACCGACAGAGATTATTGGGCCCAGAAAGAAAAGGGCAGCAGC
AGCTTCCTGCGGCCTAGCTGTGTGCCCTTCCGGGAACTGAAGAACGTGTCCGTTCTGGAAGG
CCTGAGGCAGGGCAGACTTGGCGGACCTTGTAGCTGCCACTGTCCTAGACCAAGCCAGGCC
AGACTGACCCCTGTGGATGTGGCTGGCCCATTTCTGTGTCTGGGCGACCCTGGACTGTTCCCT
CCAGTGAAGTCTAGCATCACCGAGATCAGCTGCTGCACCCTGAGCAGCGAGGAAAACGAGT
ACCTGCCTAGACCTGAATGGCAGCTGCAGTACGAGGCCGGCATGACACTCGGAGGCAAGAT
CCTGTTCTTCCTGTTCCTGCTGCTCCCTCTGAGCCCCTTCAGCCTGATCTTTCTGGTGCTGGGC
GTGCTGTCTGGCCACTCTGGAAGCAGACTGAAGAGAAGCTTCGCCGTGACCGAGCGGATCA
TCACAGGCGGCAAGAGCACATGTTCTGCCCCTGGACCTCAGTCTCTGCCCAGCACACCCTTC
AGCACATACCCTCAGTGGGTCATCCTGATCACCGAGCTGGATGGCCACAGCTACACCAGCAA
AGTGAACTGCCTCCTGCTGCAGGATGGCTTCCACGGCTGTGTGTCTATTACTGGCGCCGCTG
GCAGACGGAACCTGAGCATCTTTCTGTTTCTGATGCTGTGCAAGCTCGAGTTCCACGCCTGC
CAATGGCAGCACTACCACAGATCTGGCGAAGCCGCTGGAACCCCACTTTGGAGGCCTACCA
GAAACGTGGCCATGATGGTGCCCGACAGACAGGTGCACTACGACTTCGGCCTGAAGATCCA
GAACAAGAACTGCCCCGACGTGCTGCGGTTCCTGGATCTCAAAGTGCGCTACCTGCACAGCG
TGCCCTTCAGAGAGCTGAAAAACCAGAGAACAGCCCAGGGCGCTCCTGGAATCCATCATGC
TGCTTCTCCAGTGGCCGCCAATCTGTGCGATCCTGCCAGACATGCCCAGCATACCAGGATTC
CTTGTGGCGCTGGACAAGTGCGCGCTGGAAGAGGACCTGAAGCTGGTGGCGGAGTTCTGCA
GCCTCAAAGACCTGCTCCTGAGAAGCCTGGCTGCCCCTGTAGAAGAGGACAGCCTAGACTG
CACACCGTGAAGATGTGGCGGGCCTGCCACTTGTTTCTCCAGCCACAAGTGGGCACCCCTCC
ACCTCATACAGCCTCTGCTAGAGCACCTAGCGGCCCACCTCATCCTCACGAATCTTGTCCTGC
CGGAAGAAGGCCTGCCAGAGCCGCTCAAACATGTGCCAGACGACAGCACGGACTGCCCGGA
TGTGAAGAAGCCGGAACAGCCAGAGTGCCTAGCCTGCACCTTCATCTGCATCAGGCCGCTCT
TGGAGCCGGAAGAGGTAGAGGATGGGGAGAAGCTTGTGCCCAGGTGCCACCTTCTAGAGGC
CACTACAAGCTGATCCAGCAGCCAATCAGCCTGTTCTCCATCACCGACCGGCTGCACAAGAC
ATTCAGCCAGCTGCCTTCCGTGCATCTGTGCAGCATCACCTTCCAGTGGGGACACCCTCCTAT
CTTTTGCTCCACCAACGACATCTGCGTGACCGCCAACTTCTGTATCAGCGTGACCTTCCTGAA
GCCTTGCTTTCTGCTGCACGAGGCCTCCGCCAGCCAGTTTAAGAAGTTTGACGGCCCCTGCG
GCGAGAGAGGCGGAGGAAGAACTGCAAGAGCCCTTTGGGCCAGAGGCGACTCTGTTCTGAC
ACCAGCTCTGGACCCTCAGACACCTGTTAGGGCCCCTAGCCTGACAAGAGCTGCCGCTGCTG
TTGGAGTGCCTGGCGATTCTACTAGAAGGGCCGTGCGGCGGATGAACACCTTTTGTGGCGCA
TCTGCCTGCGACGTGTCCCTGATCGCTATGGATAGCGCCTGCGAGGAAAGAGGCGCAGCCG
GATCTCTGATCTCTTGCGAGAGCCTGTACCACCGGGAAAAGCAGCTCATTGCCATGGACAGC
GCCATCTTCGTGCAGGGCAAAGACTGGGGCGTGAAGAAGTTCATCCGGCGGGACTTTACCAT
GCCTGCCATTCTGAAGCTGCAGAAGAATTGTCTTCTAAGCCTGAACAGCAAGATGGCCCTGA
ATAGCGAGGCCCTGTCTGTGGTGTCCGAGTATGAGGCTGGAATGACCCTGGGCGAGAAGTTC
AGAGTGGGCAACTGCAAGCACCTGAAGATGACCCGGCCTACCGAGTACAACCAGAAACTGC
AAGTGAACCAGTTCAGCGAGAGCAAGCGGACCGCTCTGACCCACAACCAGGACTTCAGCAT
CTACCGGCTGTGCTGCAAGAGGGGCTCTCTGTGTCATGCTAGCCAGGCTAGAAGCCCCGCCT
TTCCTAAGCCTGTCAGACCTCTGCCTGCTCCTATCACCAGAATCACCCCTCAGCTCGGCGGCC
AGTCTGATTCATCTCAGCCACTGCTGACCACCGGCAGACCTCAAGGATGGCAAGACCAGGCT
CTGAGACACACACAGCAGGCTAGCCCAGCCTCTTGCGCCACCATCACAATACCAATACATTC
TGCCGCTCTGGGCGATCACAGCGGAGATCCTGGACCTGCCTGGGATACTTGTCCTCCTCTGC
CCCTAACTACACTGATCCCTAGGGCTCCTCCACCTTACGGCGATAGCACAGCCAGATCCTGG
CCTAGCAGATGTGGCCCTCTGGGCTACGCCTACAAGGACTTCCTGTGGTGCTTCCCCTTCTCT
CTGGTGTTCCTGCAAGAAATCCAGATCTGCTGTCACGTGTCCTGCCTGTGCTGTATCTGCTGT
AGCACCCGGATCTGTCTGGGCTGTCTGCTGGAACTGTTCCTGAGCAGAGCCCTGAGAGCACT
GCACGTGCTGTGGAACGGATTCCAGCTGCACTGCCAGGGCAACACCACACTGCAACAGCTG
GGAGAAGCCTCTCAGGCCCCAAGCGGTTCTCTGATCCCTCTCAGACTGCCCCTCCTGTGGGA
AGTGCGGGGCAATTCTAAGATGGCTCTCAACAGCCTGAACTCCATCGACGACGCCCAGCTGA
CAAGAATCGCCCCTCCAAGAAGCCACTGTTGCTTTTGGGAAGTGAACGCCCCTTTTGTGCAG
GGTAAAGATTGGGGCCTCAAAAAGTTTATCAGACGGGACTTCGAGGCTTTCCAGAGAGCAG
CTGGCGAAGGCGGACCTGGCAGAGGTGGTGCTAGAAGAGGTGCTAGAGTGCTGCAGAGCCC
ATTCTGTAGAGCTGGCGCTGGCGAATGGCTGGGCCACCAATCTCTTAGATGGGGAATGGAAC
TGGCCGCTAGCAGGCGGTTTAGCTGGGATCATCATTCTGCCGGCGGACCTCCAAGAGTGCCA
AGCGTTAGAAGCGGAGCAGCCCAGGTCCAGCCTAAAGATCCACTGCCACTGAGAACACTGG
CCGGCTGCCTTGCCAGAACAGCTCATCTTAGACCTGGCGCCGAAAGCCTGCCTCAACCTCAG
CTGCATTGCACAATCGCCAGAGAACTGCACCAGTTCGCCTTCGACCTGCTGATCAAGAGCCA
CAAGATGCACTTCTCACTGAAAGAGCACCCGCCACCGCCGTGCCCACCGCACGTTGTCGGCT
ATGGCCACCTGGATACAAGCGGCTCCTCTAGCAGTAGCTCCTGGCCTCAGCCTGACAGCTTT
GCTGCCCTGCATAGCTCCCTGAATGAGCTGGGCGAACTGTGGTTCCAGTCCAGCGAACTGTC
TCCTACTGGCGCTCCATGGCCAAGCAGAAGGCCTACTTGGAGAGGCACCACCGTGTCTCCAA
GAACCGCTACAAGCAGCGCCAGAACCTGTTGCGGCACAAAATGGCCCTCCAGCCAAGAAGC
TGCCCTCGGACTTGGAAGCGGACTGCTGAGATTCAGCTGTGGCACAGCCGCCATCAGAAGCC
ATCACCATCACCACCATTAGTAAAGGCGCGCC

The amino acid sequence of additional five neoantigen layouts for GAd20 expression are shown in SEQ ID NOs: 554, 555, 556, 623 and 624.

SEQ ID NO: 554
MGQKEQIHTLQKNSERMSKQLTRSSQAVQNLQNGGGSRSSATLPGRRRRRWLRRRRQPISVAPA
GPPRRPNQKPNPPGGARCVIMRPTWPGTSAFTKRSFAVTERIILVLGVLSGHSGSRLYEAGMTLG
GKILFFLFLLLPLSPFSLIFTEISCCTLSSEENEYLPRPEWQLQVPFRELKNVSVLEGLRQGRLGGPC
SCHCPRPSQARLTPVDVAGPFLCLGDPGLFPPVKSSITGGKSTCSAPGPQSLPSTPFSTYPQWVILIT
ELGMECTLGQVGAPSPRREEDGWRGGHSRFKADVPAPQGPCWGGQPGSAPSSAPPEQSLLDDY
WAQKEKGSSSFLRPSCDYWAQKEKISIPRTHLCWKFEMSYTVGGPPPHVHARPRHWKTDRGVP
GDSTRRAVRRMNTFYEAGMTLGEKFRVGNCKHLKMTRPNSKMALNSEALSVVSECGASACDV
SLIAMDSAFVQGKDWGVKKFIRRDFYAYKDFLWCFPFSLVFLQEIQICCHVSCLCCICCSTRICLG
CLLELFLSRALRALHVLWNGFQLHCQTEYNQKLQVNQFSESKSLYHREKQLIAMDSAICEERGA
AGSLISCETMPAILKLQKNCLLSLRTALTHNQDFSIYRLCCKRGSLCHASQARSPAFPKPVRPLPA
PITRITPQLGGQSDSSQPLLTTGRPQGWQDQALRHTQQASPASCATITIPIHSAALGDHSGDPGPA
WDTCPPLPLTTLIPRAPPPYGDSTARSWPSRCGPLGDGHSYTSKVNCLLLQDGFHGCVSITGAAG
RRNLSIFLFLMLCKLEFHACKIQNKNCPDFKKFDGPCGERGGGRTARALWARGDSVLTPALDPQ
TPVRAPSLTRAAAAVHYKLIQQPISLFSITDRLHKTFSQLPSVHLCSITFQWGHPPIFCSTNDICVTA
NFCISVTFLKPCFLLHEASASQCHLFLQPQVGTPPPHTASARAPSGPPHPHESCPAGRRPARAAQT
CARRQHGLPGCEEAGTARVPSLHLHLHQAALGAGRGRGWGEACAQVPPSRGVLRFLDLKVRYL
HSQWQHYHRSGEAAGTPLWRPTRNVPFRELKNQRTAQGAPGIHHAASPVAANLCDPARHAQHT
RIPCGAGQVRAGRGPEAGGGVLQPQRPAPEKPGCPCRRGQPRLHTVKMWRAVAMMVPDRQVH
YDFGLGNTTLQQLGEASQAPSGSLIPLRLPLLWEVRGQPDSFAALHSSLNELGEIARELHQFAFDL
LIKSHFVQGKDWGLKKFIRRDFWGMELAASRRFSWDHHSAGGPPRVPSVRSGAAQVQPKDPLP
LRTLAGCLARTAHLRPGAESLPQPQLHCTLWFQSSELSPTGAPWPSRRPTWRGTTVSPRTATSSA
RTCCGTKWPSSQEAALGLGSGLLRFSCGTAAIRKMHFSLKEHPPPPCPPEAFQRAAGEGGPGRGG
ARRGARVLQSPFCRAGAGEWLGHQSLRHVVGYGHLDTSGSSSSSSWPNSKMALNSLNSIDDAQ
LTRIAPPRSHCCFWEVNAP
SEQ ID NO: 555
MGQKEQIHTLQKNSERMSKQLTRSSQAVQNLQNGGGSRSSATLPGRRRRRWLRRRRQPISVAPA
GPPRRPNQKPNPPGGARCVIMRPTWPGTSAFTKRSFAVTERIITEISCCTLSSEENEYLPRPEWQLQ
YEAGMTLGGKILFFLFLLLPLSPFSLIFDYWAQKEKGSSSFLRPSCVPFRELKNVSVLEGLRQGRL
GGPCSCHCPRPSQARLTPVDVAGPFLCLGDPGLFPPVKSSITGGKSTCSAPGPQSLPSTPFSTYPQ
WVILITELGMECTLGQVGAPSPRREEDGWRGGHSRFKADVPAPQGPCWGGQPGSAPSSAPPEQS
LLDDYWAQKEKISIPRTHLCLVLGVLSGHSGSRLWKFEMSYTVGGPPPHVHARPRHWKTDRDG
HSYTSKVNCLLLQDGFHGCVSITGAAGRRNLSIFLFLMLCKLEFHACKIQNKNCPDFKKFDGPCG
ERGGGRTARALWARGDSVLTPALDPQTPVRAPSLTRAAAAVHYKLIQQPISLFSITDRLHKTFSQ
LPSVHLCSITFQWGHPPIFCSTNDICVTANFCISVTFLKPCFLLHEASASQCHLFLQPQVGTPPPHT
ASARAPSGPPHPHESCPAGRRPARAAQTCARRQHGLPGCEEAGTARVPSLHLHLHQAALGAGRG
RGWGEACAQVPPSRGVLRFLDLKVRYLHSQWQHYHRSGEAAGTPLWRPTRNVPFRELKNQRT
AQGAPGIHHAASPVAANLCDPARHAQHTRIPCGAGQVRAGRGPEAGGGVLQPQRPAPEKPGCP
CRRGQPRLHTVKMWRAVAMMVPDRQVHYDFGLGVPGDSTRRAVRRMNTFYEAGMTLGEKFR
VGNCKHLKMTRPNSKMALNSEALSVVSECGASACDVSLIAMDSAFVQGKDWGVKKFIRRDFYA
YKDFLWCFPFSLVFLQEIQICCHVSCLCCICCSTRICLGCLLELFLSRALRALHVLWNGFQLHCQT
EYNQKLQVNQFSESKSLYHREKQLIAMDSAICEERGAAGSLISCETMPAILKLQKNCLLSLRTALT
HNQDFSIYRLCCKRGSLCHASQARSPAFPKPVRPLPAPITRITPQLGGQSDSSQPLLTTGRPQGWQ
DQALRHTQQASPASCATITIPIHSAALGDHSGDPGPAWDTCPPLPLTTLIPRAPPPYGDSTARSWPS
RCGPLGGNTTLQQLGEASQAPSGSLIPLRLPLLWEVRGQPDSFAALHSSLNELGEIARELHQFAFD
LLIKSHFVQGKDWGLKKFIRRDFWGMELAASRRFSWDHHSAGGPPRVPSVRSGAAQVQPKDPL
PLRTLAGCLARTAHLRPGAESLPQPQLHCTLWFQSSELSPTGAPWPSRRPTWRGTTVSPRTATSS
ARTCCGTKWPSSQEAALGLGSGLLRFSCGTAAIRKMHFSLKEHPPPPCPPEAFQRAAGEGGPGRG
GARRGARVLQSPFCRAGAGEWLGHQSLRHVVGYGHLDTSGSSSSSSWPNSKMALNSLNSIDDA
QLTRIAPPRSHCCFWEVNAP
SEQ ID NO: 556
MGQKEQIHTLQKNSERMSKQLTRSSQAVQNLQNGGGSRSSATLPGRRRRRWLRRRRQPISVAPA
GPPRRPNQKPNPPGGARCVIMRPTWPGTSAFTKRSFAVTERIIDYWAQKEKGSSSFLRPSCVPFRE
LKNVSVLEGLRQGRLGGPCSCHCPRPSQARLTPVDVAGPFLCLGDPGLFPPVKSSILVLGVLSGH
SGSRLYEAGMTLGGKILFFLFLLLPLSPFSLIFTEISCCTLSSEENEYLPRPEWQLQTGGKSTCSAPG
PQSLPSTPFSTYPQWVILITELGMECTLGQVGAPSPRREEDGWRGGHSRFKADVPAPQGPCWGG
QPGSAPSSAPPEQSLLDDYWAQKEKISIPRTHLCWKFEMSYTVGGPPPHVHARPRHWKTDRDGH
SYTSKVNCLLLQDGFHGCVSITGAAGRRNLSIFLFLMLCKLEFHACKIQNKNCPDFKKFDGPCGE
RGGGRTARALWARGDSVLTPALDPQTPVRAPSLTRAAAAVHYKLIQQPISLFSITDRLHKTFSQL
PSVHLCSITFQWGHPPIFCSTNDICVTANFCISVTFLKPCFLLHEASASQCHLFLQPQVGTPPPHTAS
ARAPSGPPHPHESCPAGRRPARAAQTCARRQHGLPGCEEAGTARVPSLHLHLHQAALGAGRGR
GWGEACAQVPPSRGVLRFLDLKVRYLHSQWQHYHRSGEAAGTPLWRPTRNVPFRELKNQRTA
QGAPGIHHAASPVAANLCDPARHAQHTRIPCGAGQVRAGRGPEAGGGVLQPQRPAPEKPGCPCR
RGQPRLHTVKMWRAVAMMVPDRQVHYDFGLGVPGDSTRRAVRRMNTFYEAGMTLGEKFRVG
NCKHLKMTRPNSKMALNSEALSVVSECGASACDVSLIAMDSAFVQGKDWGVKKFIRRDFYAYK
DFLWCFPFSLVFLQEIQICCHVSCLCCICCSTRICLGCLLELFLSRALRALHVLWNGFQLHCQTEY
NQKLQVNQFSESKSLYHREKQLIAMDSAICEERGAAGSLISCETMPAILKLQKNCLLSLRTALTH
NQDFSIYRLCCKRGSLCHASQARSPAFPKPVRPLPAPITRITPQLGGQSDSSQPLLTTGRPQGWQD
QALRHTQQASPASCATITIPIHSAALGDHSGDPGPAWDTCPPLPLTTLIPRAPPPYGDSTARSWPSR
CGPLGGNTTLQQLGEASQAPSGSLIPLRLPLLWEVRGQPDSFAALHSSLNELGEIARELHQFAFDL
LIKSHFVQGKDWGLKKFIRRDFWGMELAASRRFSWDHHSAGGPPRVPSVRSGAAQVQPKDPLP
LRTLAGCLARTAHLRPGAESLPQPQLHCTLWFQSSELSPTGAPWPSRRPTWRGTTVSPRTATSSA
RTCCGTKWPSSQEAALGLGSGLLRFSCGTAAIRKMHFSLKEHPPPPCPPEAFQRAAGEGGPGRGG
ARRGARVLQSPFCRAGAGEWLGHQSLRHVVGYGHLDTSGSSSSSSWPNSKMALNSLNSIDDAQ
LTRIAPPRSHCCFWEVNAP
SEQ ID NO: 623
MGQKEQIHTLQKNSERMSKQLTRSSQAVGVPGDSTRRAVRRMNTFYEAGMTLGEKFRVGNCK
HLKMTRPNSKMALNSEALSVVSECGASACDVSLIAMDSAFVQGKDWGVKKFIRRDFYAYKDFL
WCFPFSLVFLQEIQICCHVSCLCCICCSTRICLGCLLELFLSRALRALHVLWNGFQLHCQTEYNQK
LQVNQFSESKSLYHREKQLIAMDSAICEERGAAGSLISCETMPAILKLQKNCLLSLRTALTHNQDF
SIYRLCCKRGSLCHASQARSPAFPKPVRPLPAPITRITPQLGGQSDSSQPLLTTGRPQGWQDQALR
HTQQASPASCATITIPIHSAALGDHSGDPGPAWDTCPPLPLTTLIPRAPPPYGDSTARSWPSRCGPL
GGNTTLQQLGEASQAPSGSLIPLRLPLLWEVRGQPDSFAALHSSLNELGEIARELHQFAFDLLIKS
HFVQGKDWGLKKFIRRDFWGMELAASRRFSWDHHSAGGPPRVPSVRSGAAQVQPKDPLPLRTL
AGCLARTAHLRPGAESLPQPQLHCTLWFQSSELSPTGAPWPSRRPTWRGTTVSPRTATSSARTCC
GTKWPSSQEAALGLGSGLLRFSCGTAAIRKMHFSLKEHPPPPCPPEAFQRAAGEGGPGRGGARR
GARVLQSPFCRAGAGEWLGHQSLRHVVGYGHLDTSGSSSSSSWPNSKMALNSLNSIDDAQLTRI
APPRSHCCFWEVNAPDGHSYTSKVNCLLLQDGFHGCVSITGAAGRRNLSIFLFLMLCKLEFHAC
KIQNKNCPDFKKFDGPCGERGGGRTARALWARGDSVLTPALDPQTPVRAPSLTRAAAAVHYKLI
QQPISLFSITDRLHKTFSQLPSVHLCSITFQWGHPPIFCSTNDICVTANFCISVTFLKPCFLLHEASAS
QCHLFLQPQVGTPPPHTASARAPSGPPHPHESCPAGRRPARAAQTCARRQHGLPGCEEAGTARV
PSLHLHLHQAALGAGRGRGWGEACAQVPPSRGVLRFLDLKVRYLHSQWQHYHRSGEAAGTPL
WRPTRNVPFRELKNQRTAQGAPGIHHAASPVAANLCDPARHAQHTRIPCGAGQVRAGRGPEAG
GGVLQPQRPAPEKPGCPCRRGQPRLHTVKMWRAVAMMVPDRQVHYDFGLQNLQNGGGSRSSA
TLPGRRRRRWLRRRRQPISVAPAGPPRRPNQKPNPPGGARCVIMRPTWPGTSAFTKRSFAVTERII
LVLGVLSGHSGSRLYEAGMTLGGKILFFLFLLLPLSPFSLIFTEISCCTLSSEENEYLPRPEWQLQVP
FRELKNVSVLEGLRQGRLGGPCSCHCPRPSQARLTPVDVAGPFLCLGDPGLFPPVKSSITGGKSTC
SAPGPQSLPSTPFSTYPQWVILITELGMECTLGQVGAPSPRREEDGWRGGHSRFKADVPAPQGPC
WGGQPGSAPSSAPPEQSLLDDYWAQKEKGSSSFLRPSCDYWAQKEKISIPRTHLCWKFEMSYTV
GGPPPHVHARPRHWKTDR
SEQ ID NO: 624
MGQKEQIHTLQKNSERMSKQLTRSSQAVGNTTLQQLGEASQAPSGSLIPLRLPLLWEVRGQPDSF
AALHSSLNELGEIARELHQFAFDLLIKSHFVQGKDWGLKKFIRRDFWGMELAASRRFSWDHHSA
GGPPRVPSVRSGAAQVQPKDPLPLRTLAGCLARTAHLRPGAESLPQPQLHCTLWFQSSELSPTGA
PWPSRRPTWRGTTVSPRTATSSARTCCGTKWPSSQEAALGLGSGLLRFSCGTAAIRKMHFSLKEH
PPPPCPPEAFQRAAGEGGPGRGGARRGARVLQSPFCRAGAGEWLGHQSLRHVVGYGHLDTSGS
SSSSSWPNSKMALNSLNSIDDAQLTRIAPPRSHCCFWEVNAPGVPGDSTRRAVRRMNTFYEAGM
TLGEKFRVGNCKHLKMTRPNSKMALNSEALSVVSECGASACDVSLIAMDSAFVQGKDWGVKK
FIRRDFYAYKDFLWCFPFSLVFLQEIQICCHVSCLCCICCSTRICLGCLLELFLSRALRALHVLWNG
FQLHCQTEYNQKLQVNQFSESKSLYHREKQLIAMDSAICEERGAAGSLISCETMPAILKLQKNCL
LSLRTALTHNQDFSIYRLCCKRGSLCHASQARSPAFPKPVRPLPAPITRITPQLGGQSDSSQPLLTT
GRPQGWQDQALRHTQQASPASCATITIPIHSAALGDHSGDPGPAWDTCPPLPLTTLIPRAPPPYGD
STARSWPSRCGPLGDGHSYTSKVNCLLLQDGFHGCVSITGAAGRRNLSIFLFLMLCKLEFHACKI
QNKNCPDFKKFDGPCGERGGGRTARALWARGDSVLTPALDPQTPVRAPSLTRAAAAVHYKLIQ
QPISLFSITDRLHKTFSQLPSVHLCSITFQWGHPPIFCSTNDICVTANFCISVTFLKPCFLLHEASASQ
CHLFLQPQVGTPPPHTASARAPSGPPHPHESCPAGRRPARAAQTCARRQHGLPGCEEAGTARVPS
LHLHLHQAALGAGRGRGWGEACAQVPPSRGVLRFLDLKVRYLHSQWQHYHRSGEAAGTPLWR
PTRNVPFRELKNQRTAQGAPGIHHAASPVAANLCDPARHAQHTRIPCGAGQVRAGRGPEAGGG
VLQPQRPAPEKPGCPCRRGQPRLHTVKMWRAVAMMVPDRQVHYDFGLQNLQNGGGSRSSATL
PGRRRRRWLRRRRQPISVAPAGPPRRPNQKPNPPGGARCVIMRPTWPGTSAFTKRSFAVTERIITE
ISCCTLSSEENEYLPRPEWQLQYEAGMTLGGKILFFLFLLLPLSPFSLIFDYWAQKEKGSSSFLRPS
CVPFRELKNVSVLEGLRQGRLGGPCSCHCPRPSQARLTPVDVAGPFLCLGDPGLFPPVKSSITGG
KSTCSAPGPQSLPSTPFSTYPQWVILITELGMECTLGQVGAPSPRREEDGWRGGHSRFKADVPAP
QGPCWGGQPGSAPSSAPPEQSLLDDYWAQKEKISIPRTHLCLVLGVLSGHSGSRLWKFEMSYTV
GGPPPHVHARPRHWKTDR

The amino acid sequence of additional five neoantigen layouts for MVA expression are shown in SEQ ID NOs: 557, 558, 559, 625 and 626.

SEQ ID NO: 557
MGQKEQIHTLQKNSERMSKQLTRSSQAVQNLQNGGGSRSSATLPGRRRRRWLRRRRQPISVAPA
GPPRRPNQKPNPPGGARCVIMRPTWPGTSAFTGMECTLGQVGAPSPRREEDGWRGGHSRFKAD
VPAPQGPCWGGQPGSAPSSAPPEQSLLDDYWAQKEKISIPRTHLCWKFEMSYTVGGPPPHVHAR
PRHWKTDRDYWAQKEKGSSSFLRPSCVPFRELKNVSVLEGLRQGRLGGPCSCHCPRPSQARLTP
VDVAGPFLCLGDPGLFPPVKSSITEISCCTLSSEENEYLPRPEWQLQYEAGMTLGGKILFFLFLLLP
LSPFSLIFLVLGVLSGHSGSRLKRSFAVTERIITGGKSTCSAPGPQSLPSTPFSTYPQWVILITELDGH
SYTSKVNCLLLQDGFHGCVSITGAAGRRNLSIFLFLMLCKLEFHACCHLFLQPQVGTPPPHTASA
RAPSGPPHPHESCPAGRRPARAAQTCARRQHGLPGCEEAGTARVPSLHLHLHQAALGAGRGRG
WGEACAQVPPSRGHYKLIQQPISLFSITDRLHKTFSQLPSVHLCSITFQWGHPPIFCSTNDICVTAN
FCISVTFLKPCFLLHEASASQVAMMVPDRQVHYDFGLKIQNKNCPDVLRFLDLKVRYLHSVPFR
ELKNQRTAQGAPGIHHAASPVAANLCDPARHAQHTRIPCGAGQVRAGRGPEAGGGVLQPQRPA
PEKPGCPCRRGQPRLHTVKMWRAQWQHYHRSGEAAGTPLWRPTRNFKKFDGPCGERGGGRTA
RALWARGDSVLTPALDPQTPVRAPSLTRAAAAVGVPGDSTRRAVRRMNTFCGASACDVSLIAM
DSACEERGAAGSLISCESLYHREKQLIAMDSAIFVQGKDWGVKKFIRRDFTMPAILKLQKNCLLS
LNSKMALNSEALSVVSEYEAGMTLGEKFRVGNCKHLKMTRPTEYNQKLQVNQFSESKRTALTH
NQDFSIYRLCCKRGSLCHASQARSPAFPKPVRPLPAPITRITPQLGGQSDSSQPLLTTGRPQGWQD
QALRHTQQASPASCATITIPIHSAALGDHSGDPGPAWDTCPPLPLTTLIPRAPPPYGDSTARSWPSR
CGPLGYAYKDFLWCFPFSLVFLQEIQICCHVSCLCCICCSTRICLGCLLELFLSRALRALHVLWNG
FQLHCQGNTTLQQLGEASQAPSGSLIPLRLPLLWEVRGNSKMALNSLNSIDDAQLTRIAPPRSHC
CFWEVNAPFVQGKDWGLKKFIRRDFEAFQRAAGEGGPGRGGARRGARVLQSPFCRAGAGEWL
GHQSLRWGMELAASRRFSWDHHSAGGPPRVPSVRSGAAQVQPKDPLPLRTLAGCLARTAHLRP
GAESLPQPQLHCTIARELHQFAFDLLIKSHKMHFSLKEHPPPPCPPHVVGYGHLDTSGSSSSSSWP
QPDSFAALHSSLNELGELWFQSSELSPTGAPWPSRRPTWRGTTVSPRTATSSARTCCGTKWPSSQ
EAALGLGSGLLRFSCGTAAIR
SEQ ID NO: 558
MGQKEQIHTLQKNSERMSKQLTRSSQAVDGHSYTSKVNCLLLQDGFHGCVSITGAAGRRNLSIF
LFLMLCKLEFHACQWQHYHRSGEAAGTPLWRPTRNVAMMVPDRQVHYDFGLVLRFLDLKVRY
LHSKIQNKNCPDVPFRELKNQRTAQGAPGIHHAASPVAANLCDPARHAQHTRIPCGAGQVRAGR
GPEAGGGVLQPQRPAPEKPGCPCRRGQPRLHTVKMWRACHLFLQPQVGTPPPHTASARAPSGPP
HPHESCPAGRRPARAAQTCARRQHGLPGCEEAGTARVPSLHLHLHQAALGAGRGRGWGEACA
QVPPSRGHYKLIQQPISLFSITDRLHKTFSQLPSVHLCSITFQWGHPPIFCSTNDICVTANFCISVTFL
KPCFLLHEASASQFKKFDGPCGERGGGRTARALWARGDSVLTPALDPQTPVRAPSLTRAAAAVQ
NLQNGGGSRSSATLPGRRRRRWLRRRRQPISVAPAGPPRRPNQKPNPPGGARCVIMRPTWPGTS
AFTGMECTLGQVGAPSPRREEDGWRGGHSRFKADVPAPQGPCWGGQPGSAPSSAPPEQSLLDD
YWAQKEKISIPRTHLCWKFEMSYTVGGPPPHVHARPRHWKTDRDYWAQKEKGSSSFLRPSCVP
FRELKNVSVLEGLRQGRLGGPCSCHCPRPSQARLTPVDVAGPFLCLGDPGLFPPVKSSITEISCCTL
SSEENEYLPRPEWQLQYEAGMTLGGKILFFLFLLLPLSPFSLIFLVLGVLSGHSGSRLKRSFAVTER
IITGGKSTCSAPGPQSLPSTPFSTYPQWVILITELGVPGDSTRRAVRRMNTFCGASACDVSLIAMDS
ACEERGAAGSLISCESLYHREKQLIAMDSAIFVQGKDWGVKKFIRRDFTMPAILKLQKNCLLSLN
SKMALNSEALSVVSEYEAGMTLGEKFRVGNCKHLKMTRPTEYNQKLQVNQFSESKRTALTHNQ
DFSIYRLCCKRGSLCHASQARSPAFPKPVRPLPAPITRITPQLGGQSDSSQPLLTTGRPQGWQDQA
LRHTQQASPASCATITIPIHSAALGDHSGDPGPAWDTCPPLPLTTLIPRAPPPYGDSTARSWPSRCG
PLGYAYKDFLWCFPFSLVFLQEIQICCHVSCLCCICCSTRICLGCLLELFLSRALRALHVLWNGFQ
LHCQGNTTLQQLGEASQAPSGSLIPLRLPLLWEVRGNSKMALNSLNSIDDAQLTRIAPPRSHCCF
WEVNAPFVQGKDWGLKKFIRRDFEAFQRAAGEGGPGRGGARRGARVLQSPFCRAGAGEWLGH
QSLRWGMELAASRRFSWDHHSAGGPPRVPSVRSGAAQVQPKDPLPLRTLAGCLARTAHLRPGA
ESLPQPQLHCTIARELHQFAFDLLIKSHKMHFSLKEHPPPPCPPHVVGYGHLDTSGSSSSSSWPQP
DSFAALHSSLNELGELWFQSSELSPTGAPWPSRRPTWRGTTVSPRTATSSARTCCGTKWPSSQEA
ALGLGSGLLRFSCGTAAIR
SEQ ID NO: 559
MGQKEQIHTLQKNSERMSKQLTRSSQAVGNTTLQQLGEASQAPSGSLIPLRLPLLWEVRGNSKM
ALNSLNSIDDAQLTRIAPPRSHCCFWEVNAPFVQGKDWGLKKFIRRDFEAFQRAAGEGGPGRGG
ARRGARVLQSPFCRAGAGEWLGHQSLRWGMELAASRRFSWDHHSAGGPPRVPSVRSGAAQVQ
PKDPLPLRTLAGCLARTAHLRPGAESLPQPQLHCTIARELHQFAFDLLIKSHKMHFSLKEHPPPPC
PPHVVGYGHLDTSGSSSSSSWPQPDSFAALHSSLNELGELWFQSSELSPTGAPWPSRRPTWRGTT
VSPRTATSSARTCCGTKWPSSQEAALGLGSGLLRFSCGTAAIRDGHSYTSKVNCLLLQDGFHGC
VSITGAAGRRNLSIFLFLMLCKLEFHACQWQHYHRSGEAAGTPLWRPTRNVAMMVPDRQVHYD
FGLKIQNKNCPDVLRFLDLKVRYLHSVPFRELKNQRTAQGAPGIHHAASPVAANLCDPARHAQH
TRIPCGAGQVRAGRGPEAGGGVLQPQRPAPEKPGCPCRRGQPRLHTVKMWRACHLFLQPQVGT
PPPHTASARAPSGPPHPHESCPAGRRPARAAQTCARRQHGLPGCEEAGTARVPSLHLHLHQAAL
GAGRGRGWGEACAQVPPSRGHYKLIQQPISLFSITDRLHKTFSQLPSVHLCSITFQWGHPPIFCST
NDICVTANFCISVTFLKPCFLLHEASASQFKKFDGPCGERGGGRTARALWARGDSVLTPALDPQT
PVRAPSLTRAAAAVGVPGDSTRRAVRRMNTFCGASACDVSLIAMDSATEYNQKLQVNQFSESK
YEAGMTLGEKFRVGNCKHLKMTRPTMPAILKLQKNCLLSLNSKMALNSEALSVVSECEERGAA
GSLISCESLYHREKQLIAMDSAIFVQGKDWGVKKFIRRDFRTALTHNQDFSIYRLCCKRGSLCHA
SQARSPAFPKPVRPLPAPITRITPQLGGQSDSSQPLLTTGRPQGWQDQALRHTQQASPASCATITIP
IHSAALGDHSGDPGPAWDTCPPLPLTTLIPRAPPPYGDSTARSWPSRCGPLGYAYKDFLWCFPFSL
VFLQEIQICCHVSCLCCICCSTRICLGCLLELFLSRALRALHVLWNGFQLHCQQNLQNGGGSRSSA
TLPGRRRRRWLRRRRQPISVAPAGPPRRPNQKPNPPGGARCVIMRPTWPGTSAFTGMECTLGQV
GAPSPRREEDGWRGGHSRFKADVPAPQGPCWGGQPGSAPSSAPPEQSLLDDYWAQKEKISIPRT
HLCWKFEMSYTVGGPPPHVHARPRHWKTDRDYWAQKEKGSSSFLRPSCVPFRELKNVSVLEGL
RQGRLGGPCSCHCPRPSQARLTPVDVAGPFLCLGDPGLFPPVKSSITEISCCTLSSEENEYLPRPEW
QLQYEAGMTLGGKILFFLFLLLPLSPFSLIFLVLGVLSGHSGSRLKRSFAVTERIITGGKSTCSAPGP
QSLPSTPFSTYPQWVILITEL
SEQ ID NO: 625
MGQKEQIHTLQKNSERMSKQLTRSSQAVGNTTLQQLGEASQAPSGSLIPLRLPLLWEVRGNSKM
ALNSLNSIDDAQLTRIAPPRSHCCFWEVNAPFVQGKDWGLKKFIRRDFEAFQRAAGEGGPGRGG
ARRGARVLQSPFCRAGAGEWLGHQSLRWGMELAASRRFSWDHHSAGGPPRVPSVRSGAAQVQ
PKDPLPLRTLAGCLARTAHLRPGAESLPQPQLHCTIARELHQFAFDLLIKSHKMHFSLKEHPPPPC
PPHVVGYGHLDTSGSSSSSSWPQPDSFAALHSSLNELGELWFQSSELSPTGAPWPSRRPTWRGTT
VSPRTATSSARTCCGTKWPSSQEAALGLGSGLLRFSCGTAAIRQNLQNGGGSRSSATLPGRRRRR
WLRRRRQPISVAPAGPPRRPNQKPNPPGGARCVIMRPTWPGTSAFTGMECTLGQVGAPSPRREE
DGWRGGHSRFKADVPAPQGPCWGGQPGSAPSSAPPEQSLLDDYWAQKEKISIPRTHLCWKFEM
SYTVGGPPPHVHARPRHWKTDRDYWAQKEKGSSSFLRPSCVPFRELKNVSVLEGLRQGRLGGP
CSCHCPRPSQARLTPVDVAGPFLCLGDPGLFPPVKSSITEISCCTLSSEENEYLPRPEWQLQYEAG
MTLGGKILFFLFLLLPLSPFSLIFLVLGVLSGHSGSRLKRSFAVTERIITGGKSTCSAPGPQSLPSTPF
STYPQWVILITELDGHSYTSKVNCLLLQDGFHGCVSITGAAGRRNLSIFLFLMLCKLEFHACQWQ
HYHRSGEAAGTPLWRPTRNVAMMVPDRQVHYDFGLVLRFLDLKVRYLHSKIQNKNCPDVPFRE
LKNQRTAQGAPGIHHAASPVAANLCDPARHAQHTRIPCGAGQVRAGRGPEAGGGVLQPQRPAP
EKPGCPCRRGQPRLHTVKMWRACHLFLQPQVGTPPPHTASARAPSGPPHPHESCPAGRRPARAA
QTCARRQHGLPGCEEAGTARVPSLHLHLHQAALGAGRGRGWGEACAQVPPSRGHYKLIQQPISL
FSITDRLHKTFSQLPSVHLCSITFQWGHPPIFCSTNDICVTANFCISVTFLKPCFLLHEASASQFKKF
DGPCGERGGGRTARALWARGDSVLTPALDPQTPVRAPSLTRAAAAVGVPGDSTRRAVRRMNTF
CGASACDVSLIAMDSACEERGAAGSLISCESLYHREKQLIAMDSAIFVQGKDWGVKKFIRRDFT
MPAILKLQKNCLLSLNSKMALNSEALSVVSEYEAGMTLGEKFRVGNCKHLKMTRPTEYNQKLQ
VNQFSESKRTALTHNQDFSIYRLCCKRGSLCHASQARSPAFPKPVRPLPAPITRITPQLGGQSDSSQ
PLLTTGRPQGWQDQALRHTQQASPASCATITIPIHSAALGDHSGDPGPAWDTCPPLPLTTLIPRAP
PPYGDSTARSWPSRCGPLGYAYKDFLWCFPFSLVFLQEIQICCHVSCLCCICCSTRICLGCLLELFL
SRALRALHVLWNGFQLHCQ
SEQ ID NO: 626
MGQKEQIHTLQKNSERMSKQLTRSSQAVDGHSYTSKVNCLLLQDGFHGCVSITGAAGRRNLSIF
LFLMLCKLEFHACVPFRELKNQRTAQGAPGIHHAASPVAANLCDPARHAQHTRIPCGAGQVRAG
RGPEAGGGVLQPQRPAPEKPGCPCRRGQPRLHTVKMWRAQWQHYHRSGEAAGTPLWRPTRNK
IQNKNCPDVAMMVPDRQVHYDFGLVLRFLDLKVRYLHSCHLFLQPQVGTPPPHTASARAPSGPP
HPHESCPAGRRPARAAQTCARRQHGLPGCEEAGTARVPSLHLHLHQAALGAGRGRGWGEACA
QVPPSRGHYKLIQQPISLFSITDRLHKTFSQLPSVHLCSITFQWGHPPIFCSTNDICVTANFCISVTFL
KPCFLLHEASASQFKKFDGPCGERGGGRTARALWARGDSVLTPALDPQTPVRAPSLTRAAAAVQ
NLQNGGGSRSSATLPGRRRRRWLRRRRQPISVAPAGPPRRPNQKPNPPGGARCVIMRPTWPGTS
AFTGMECTLGQVGAPSPRREEDGWRGGHSRFKADVPAPQGPCWGGQPGSAPSSAPPEQSLLDD
YWAQKEKISIPRTHLCWKFEMSYTVGGPPPHVHARPRHWKTDRDYWAQKEKGSSSFLRPSCVP
FRELKNVSVLEGLRQGRLGGPCSCHCPRPSQARLTPVDVAGPFLCLGDPGLFPPVKSSITEISCCTL
SSEENEYLPRPEWQLQYEAGMTLGGKILFFLFLLLPLSPFSLIFLVLGVLSGHSGSRLKRSFAVTER
IITGGKSTCSAPGPQSLPSTPFSTYPQWVILITELGNTTLQQLGEASQAPSGSLIPLRLPLLWEVRGN
SKMALNSLNSIDDAQLTRIAPPRSHCCFWEVNAPFVQGKDWGLKKFIRRDFEAFQRAAGEGGPG
RGGARRGARVLQSPFCRAGAGEWLGHQSLRWGMELAASRRFSWDHHSAGGPPRVPSVRSGAA
QVQPKDPLPLRTLAGCLARTAHLRPGAESLPQPQLHCTIARELHQFAFDLLIKSHKMHFSLKEHPP
PPCPPHVVGYGHLDTSGSSSSSSWPQPDSFAALHSSLNELGELWFQSSELSPTGAPWPSRRPTWR
GTTVSPRTATSSARTCCGTKWPSSQEAALGLGSGLLRFSCGTAAIRGVPGDSTRRAVRRMNTFC
GASACDVSLIAMDSACEERGAAGSLISCESLYHREKQLIAMDSAIFVQGKDWGVKKFIRRDFYEA
GMTLGEKFRVGNCKHLKMTRPTMPAILKLQKNCLLSLNSKMALNSEALSVVSETEYNQKLQVN
QFSESKRTALTHNQDFSIYRLCCKRGSLCHASQARSPAFPKPVRPLPAPITRITPQLGGQSDSSQPL
LTTGRPQGWQDQALRHTQQASPASCATITIPIHSAALGDHSGDPGPAWDTCPPLPLTTLIPRAPPP
YGDSTARSWPSRCGPLGYAYKDFLWCFPFSLVFLQEIQICCHVSCLCCICCSTRICLGCLLELFLSR
ALRALHVLWNGFQLHCQ

SEQ ID NO: 713 The polynucleotide sequence of the full GAd20 incorporating the GAd20 expression cassette

catcatcaataatataccttattttggattgaggccaatatgataatgaggtgggcggggcgaggcggggcgggtgacgtaggacgcgcga
gtagggttgggaggtgtggcggaagtgtggcatttgcaagtgggaggagctgacatgcaatcttccgtcgcggaaaatgtgacgtttttgat
gagcgccgcctacctccggaagtgccaattttcgcgcgatttcaccggatatcgtagtaattttgggcgggaccatgtaagatttggccatttt
cgcgcgaaaagtgaaacggggaagtgaaaactgaataatagggcgttagtcatagcgcgtaatatttaccgagggccgagggactttgac
cgattacgtggaggactcgcccaggtgttttttacgtgaatttccgcgttccgggtcaaagtctccgtttttattgtcgccgtcatctgacgggcc
gccattgcatacgttgtatccatatcataatatgtacatttatattggctcatgtccaacattaccgccatgttgacattgattattgactagttattaa
tagtaatcaattacggggtcattagttcatagcccatatatggagttccgcgttacataacttacggtaaatggcccgcctggctgaccgccca
acgacccccgcccattgacgtcaataatgacgtatgttcccatagtaacgccaatagggactttccattgacgtcaatgggtggagtatttacg
gtaaactgcccacttggcagtacatcaagtgtatcatatgccaagtacgccccctattgacgtcaatgacggtaaatggcccgcctggcatta
tgcccagtacatgaccttatgggactttcctacttggcagtacatctacgtattagtcatcgctattaccatggtgatgcggttttggcagtacatc
aatgggcgtggatagcggtttgactcacggggatttccaagtctccaccccattgacgtcaatgggagtttgttttggcaccaaaatcaacgg
gactttccaaaatgtcgtaacaactccgccccattgacgcaaatgggcggtaggcgtgtacggtgggaggtctatataagcagagctctccc
tatcagtgatagagatctccctatcagtgatagagatcgtcgacgagctcgtttagtgaaccgtcagatcgcctggagacgccatccacgctg
ttttgacctccatagaagacaccgggaccgatccagcctccgcggccgggaacggtgcattggaacgcggattccccgtgccaagagtga
gatcttccgtttatctaggtaccagatatcagaattcggatcccgcgacttcgccgccatgggccagaaagagcagatccacacactgcaga
aaaacagcgagcggatgagcaagcagctgaccagatcttctcaggccgtgcagaacctgcagaacggcggaggctctagaagctctgct
acacttcctggcaggcggcggagaagatggctgagaagaaggcggcagcctatctctgtggctcctgctggacctcctagacggcccaa
ccagaagcctaatcctcctggcggagccagatgcgtgatcatgaggcctacatggcctggcaccagcgccttcaccaagagaagctttgc
cgtgaccgagcggatcatcgactattgggctcaaaaagagaagggcagcagcagcttcctgcggcctagctgtgattattgggcccagaa
agaaaagatcagcatccccagaacacacctgtgcctggtgctgggagtgctgtctggacactctggcagcagactgtatgaggccggcat
gacactcggcggcaagatcctgttcttcctgttcctgctgctccctctgagccccttcagcctgatcttcaccgagatcagctgctgcaccctg
agcagcgaggaaaacgagtacctgcctagacctgagtggcagctgcaggtccccttcagagagctgaagaacgtaccgtgctggaagg
cctgagacagggcagacttggcggcccttgtagctgtcactgccccagacctagtcaggccagactgacacctgtggatgtggccggacc
tttcctgtgtctgggagatcctggcctgtttccacctgtgaagtccagcatcacaggcggcaagtccacatgttctgcccctggacctcagag
cctgcctagcacacccttcagcacataccctcagtgggtcatcctgatcaccgaactcggcatggaatgcaccctgggacaagtgggagcc
ccatctcctagaagagaagaggatggctggcgcggaggccactctagattcaaagctgatgtgcccgctcctcagggcccttgttgggga
ggacaacctggatctgccccatcttctgccccacctgaacagtccctgctggattggaagttcgagatgagctacaccgtcggcggacctcc
acctcatgttcatgccagacctcggcactggaaaaccgacagagatggccacagctacaccagcaaagtgaactgcctcctgctgcagga
tggcttccacggctgtgtgtctattactggcgccgctggcagacggaacctgagcatctttctgtttctgatgctgtgcaagctcgagttccac
gcctgcaagatccagaacaagaactgccccgacttcaagaagttcgacggcccttgcggagaaagaggcggaggcagaacagctagag
ccctttgggctagaggcgacagcgttctgacaccagctctggaccctcagacacctgttagggcccctagcctgacaagagctgccgccg
ctgtgcactacaagctgatccagcagccaatcagcctgttcagcatcaccgaccggctgcacaagacattcagccagctgccaagcgtgc
acctgtgctccatcaccttccagtggggacaccctcctatcttttgctccaccaacgacatctgcgtgaccgccaacttctgtatcagcgtgac
cttcctgaagccttgctttctgctgcacgaggccagcgcctctcagtgccacttgtttctgcagccccaagtgggcacacctcctccacataca
gcctctgctagagcacctagcggccctccacatcctcacgaatcttgtcctgccggaagaaggcctgccagagccgctcaaacatgtgcca
gacgacagcacggactgcctggatgtgaagaggctggaacagccagagtgcctagcctgcacctccatctgcatcaggctgctcttggag
ccggaagaggtagaggatggggcgaagcttgtgctcaggtgccaccttctagaggcgtgctgagattcctggacctgaaagtgcgctacc
tgcacagccagtggcagcactatcacagatctggcgaagccgccggaacacccctttggaggccaacaagaaacgtgcccttccgggaa
ctgaagaaccagagaacagctcagggcgctcctggaatccaccatgctgcttctccagtggccgccaacctgtgtgatcctgccagacatg
cccagcacaccaggattccttgtggcgctggacaagtgcgcgctggaagaggacctgaagcaggcggaggtgttctgcaacctcaaaga
cccgctcctgagaagcctggctgcccttgcagaagaggacagcctagactgcacaccgtgaaaatgtggcgagccgtggccatgatggt
gcccgatagacaggtccactacgactttggactgggcgtgccaggcgatagcactcggagagccgtcagacggatgaacaccttttacga
agccgggatgaccctgggcgagaagttcagagtgggcaactgcaagcacctgaagatgacccggcctaacagcaagatggccctgaat
agcgaggccctgtctgtggtgtctgaatgtggcgcctctgcctgtgacgtgtccctgatcgctatggactccgcctttgtgcagggcaaagac
tggggcgtgaagaagttcatccggcgggacttctacgcctacaaggacttcctgtggtgcttccccttctctctggtgttcctgcaagagatcc
agatctgctgtcatgtgtcctgcctgtgctgcatctgctgtagcaccagaatctgcctgggctgtctgctggaactgttcctgagcagagccct
gagagcactgcacgtgctgtggaacggattccagctgcactgccagaccgagtacaaccagaaactgcaagtgaaccagttcagcgaga
gcaagagcctgtaccaccgggaaaagcagctcattgccatggacagcgccatctgcgaagagagaggcgccgcaggatctctgatctcc
tgcgaaacaatgcccgccatcctgaagctgcagaagaattgcctcctaagcctgcgaaccgctctgacacacaaccaggacttcagcatct
acagactgtgttgcaagcggggctccctgtgccatgcaagccaagctagaagccccgcctttcctaaacctgtgcgacctctgccagctcc
aatcaccagaattacccctcagctcggcggccagagcgattcatctcaacctctgctgaccaccggcagacctcaaggctggcaagacca
agctctgagacacacccagcaggctagccctgcctcttgtgccaccatcacaatccccatccactctgccgctctgggcgatcattctggcg
atcctggaccagcctgggacacatgtcctccactgccactcacaacactgatccctagggctcctccaccttacggcgattctaccgctaga
agctggcccagcagatgtggaccactcggaggcaacacaaccctccagcaactgggagaagcctctcaggctcctagcggctctctgatc
cctctcagactgcctctcctgtgggaagttcggggccagcctgattcttttgccgcactgcacagctccctgaacgagctgggagagatcgc
tagagagctgcaccagttcgccttcgacctgctgatcaagagccacttcgtgcaaggcaaggattggggcctcaaaaagtttatccgcaga
gacttctggggcatggaactggccgccagcagaagattcagctgggatcatcatagcgcaggcggcccacctagagtgccatctgttaga
agcggagctgcccaggtgcagcctaaagatcctctgccactgagaacactggccggctgccttgctagaacagcccatcttagacctggc
gccgagtctctgcctcagccacaactgcactgtaccctgtggttccagtccagcgagctgtctcctactggtgccccttggccatctagacgc
cctacttggagaggcaccaccgtgtcaccaagaaccgccacaagcagcgccagaacctgttgtggcacaaagtggccctccagccaaga
agccgctctcggacttggaagcggactgctgaggttctcttgtggaaccgccgccattcggaagatgcactttagcctgaaagaacaccctc
caccaccttgtcctccagaggctttccaaagagctgctggcgaaggcggacctggtagaggtggtgctagaagaggtgctagggtgctgc
agagcccattctgtagagcaggcgcaggcgaatggctgggccatcagagtctgagacatgtcgtcggctacggccacctggatacaagc
ggaagcagctctagctccagctggcctaactcaaaaatggctctgaacagcctgaactccatcgacgacgcccagctgacaagaatcgcc
cctcctagatctcactgctgcttttgggaagtgaacgccccaagccatcaccatcaccaccattagtaaaggcgcgcctagcggccgcgatc
tgctgtgccttctagttgccagccatctgttgtttgcccctcccccgtgccttccttgaccctggaaggtgccactcccactgtcctttcctaataa
aatgaggaaattgcatcgcattgtctgagtaggtgtcattctattctggggggtggggtggggcaggacagcaagggggaggattgggaa
gacaatagcaggcatgctggggatgcggtgggctctatggccgggccgcgatcgcgcttaggcctgaccatctggtgctggcctgcacca
gggccgagtttgggtctagcgatgaggataccgattgaggtgggtaaggtgggcgtggctagcagggtgggcgtgtataaattgggggtc
taaggggtctctctgtttgtcttgcaacagccgccgccatgagcgacaccggcaacagctttgatggaagcatctttagtccctatctgacagt
gcgcatgcctcactgggccggagtgcgtcagaatgtgatgggttccaacgtggatggacgtcccgttctgccttcaaattcgtctactatggc
ctacgcgaccgtgggaggaactccgctggacgccgcgacctccgccgccgcctccgccgccgccgcgaccgcgcgcagcatggctac
ggacctttacagctctttggtggcgagcagcgcggcctctcgcgcgtctgctcgggatgagaaactgactgctctgctgcttaaactggaag
acttgacccgggagctgggtcaactgacccagcaggtttccagcttgcgtgagagcagccttgcctccccctaatggcccataatataaata
aaagccagtctgtttggattaagcaagtgtatgttctttatttaactctccgcgcgcggtaagcccgggaccagcggtctcggtcgtttagggt
gcggtggattttttccaacacgtggtacaggtggctctggatgtttagatacatgggcatgagtccatccctggggtggaggtagcaccactg
cagagcttcgtgctcgggggtggtgttgtatatgatccagtcgtagcaggagcgctgggcgtggtgctgaaaaatgtccttaagcaagagg
cttatagctagggggaggcccttggtgtaagtgtttacaaatctgcttagctgggaggggtgcatccggggggatatgatgtgcatcttggac
tggatttttaggttggctatgttcccgcccagatcccttctgggattcatgttgtgcaggaccaccagcacggtatatccagtgcacttgggaaa
tttatcgtggagcttagacgggaatgcatggaagaacttggagacgcccttgtggcctcccagattttccatacattcgtccatgatgatggca
atgggcccgtgggaagctgcctgagcaaaaacgtttctggcatcgctcacatcgtagttatgttccagggtgaggtcatcataggacatcttt
acgaatcgggggcgaagggtcccggactgggggatgatggtaccctcgggccccggggcgtagttcccctcacagatctgcatctccca
ggctttcatttcagagggagggatcatatccacctgcggggcgatgaaaaagacagtttctggcgcaggggagattaactgggatgagag
caggtttctgagcagctgtgactttccacagccggtgggcccatatatcacgcctatcaccggctgcagctggtagttaagagagctgcagc
tgccgtcctcccggagcaggggggccacctcgttgagcatatccctgacgtggatgttctccctgaccagttccgccagaaggcgctcgcc
gcccagcgaaagcagctcttgcaaggaagcaaaatttttcagcggtttcaggccatcggccgtgggcatgtttttcagcgtctgggtcagca
gctccagcctgtcccagagctcggtgatgtgctctacggcatctcgatccagcagatctcctcgtttcgcgggttggggcggctttcgctgta
gggcaccagccgatgggcgtccagcggggccagagtcatgtccttccatgggcgcagggtcctcgtcagggtggtctgggtcacggtga
aggggtgcgctccgggttgggcactggccagggtgcgcttgaggctggttctgctggtgctgaatcgctgccgctcttcgccctgcgcgtc
ggccaggtagcatttgaccatggtctcgtagtcgagaccctcggcggcgtgccccttggcgcggagctttcccttggaggtggcgccgca
cgaggggcactgcaggctcttcagggcgtagagcttgggagcgagaaacacggactctggggagtaggcgtccgcgccgcaggccga
gcagaccgtctcgcattccaccagccaagtgagttccgggcggtcagggtcaaaaaccaggttgcccccatgctttttgatgcgtttcttacc
ttggctctccatgaggcggtgtcccttctcggtgacgaagaggctgtccgtgtccccgtagaccgacttcaggggcctgtcttccagcggag
tgcctctgtcctcctcgtagagaaactctgaccactctgagacgaaggcccgcgtccaggccaggacgaaggaggccacgtgggaggg
gtagcggtcgttgtccactagcgggtccaccttctccagggtgtgcaggcacatgtccccctcctccgcgtccagaaaagtgattggcttgta
ggtgtaggacacgtgaccgggggttcccaacgggggggtataaaagggggtgggtgccctttcatcttcactctcttccgcatcgctgtctg
cgagagccagctgctggggtaagtattccctctcgaaggcgggcatgacctcagcgctcaggttgtcagtttctaaaaatgaggaggatttg
atgttcacctgtccggaggtgatacctttgagggtacctgggtccatctggtcagaaaacactattatagttatcaagcttggtggcgaatgac
ccgtagagggcgttggagagcagcttggcgatggagcgcagggtctggtttttgtcgcggtcggctcgctccttggccgcgatgttgagttg
cacgtactcgcgggccacgcacttccactcggggaacacggtggtgcgctcgtctgggatcaggcgcaccctccagccgcggttgtgca
gggtgaccatgtcgacgctggtggcgacctcaccgcgcagacgctcgttggtccagcagaggcggccgcccttgcgcgagcagaaggg
gggtagggggtccagctggtcctcgtttggggggtccgcgtcgatggtaaagaccccggggagcaggcgcgggtcaaagtagtcgatct
tgcaagcttgcatgtccagagcccgctgccattcgcgggcggcgagcgcgcgctcgtaggggttgaggggcgggccccagggcatgg
ggtgggtgagcgcggaggcgtacatgccgcagatgtcatacacgtacaggggttccctgaggataccgaggtaggtggggtagcagcg
ccccccgcggatgctggcgcgcacgtagtcatagagctcgtgggagggggccagcatgttgggcccgaggttggtgcgctgggggcgc
tcggcgcggaagacgatctgcctgaagatggcgtgggagttggaggagatggtgggccgctggaagacgttgaagcttgcttcttgcaag
cccacggagtccctgacgaaggaggcgtaggactcgcgcagcttgtgcaccagctcggcggtgacctggacgtcgagcgcacagtagt
cgagggtctcgcggatgatgtcatacctatcctcccccttctttttccacagctcgcggttgaggacgaactcttcgcggtctttccagtactctt
ggaggggaaacccgtccgtgtccgaacggtaagagcctagcatgtagaactggttgacggcctggtaggggcagcagcccttctccacg
ggcagcgcgtaggcctgcgccgccttgcggagggaggtgtgggtgagggcgaaagtgtccctgaccatgactttgaggtattgatgtctg
aagtctgtgtcatcgcagccgccctgttcccacagggtgtagtccgtgcgctttttggagcgcgggttgggcagggagaaggtgaggtcatt
gaagaggatcttccccgctcgaggcatgaagtttctggtgatgcgaaagggccctgggaccgaggagcggttgttgatgacctgggcggc
caggacgatctcgtcaaagccgtttatgttgtgtcccacgatgtagagctccaggaagcggggctggcccttgatggaggggagctttttaa
gttcctcgtaggtaagctcctcgggcgattccaggccgtgctcctccagggcccagtcttgcaagtgagggttggccgccaggaaggatcg
ccagaggtcgcgggccatgagggtctgcaggcggtcgcggaaggttctgaactgccgccccacggccattttttcgggggtgatgcagta
gaaggtgagggggtctttctcccaggggtcccatctgagctctcgggcgaggtcgcgcgcggcagcgaccagagcctcgtcgcccccca
gtttcatgaccagcatgaagggcacgagttgcttgccaaaggctcccatccaagtgtaggtttctacatcgtaggtgacaaagaggcgctcc
gtgcgaggatgagagccgattgggaagaactggatctcccgccaccagttggaggattggctgttgatgtggtgaaagtagaagtcccgtc
tgcgggccgagcactcgtgctggcttttgtaaaagcgaccgcagtactggcagcgctgcacgggttgtatatcttgcacgaggtgaacctg
gcgacctctgacgaggaagcgcagcgggaatctaagtcccccgcctggggtcccgtgtggctggtggtcttttactttggttgtctggccgc
cagcatctgtctcctggagggcgatggtggaacagaccaccacgccgcgagagccgcaggtccagatctcggcgctcggcgggcgga
gtttgatgacgacatcgcgcacattggagctgtccatggtctccagctcccgcggcggcaggtcagccgggagttccctggaggttcacct
cgcagagacgggtcaaggcgcggacagtgttgagatggtatctgatttcaaggggcatgttggaggcggagtcgatggcttgcaggagg
ccgcagccccggggggccacgatggttccccgcggggcgcgaggggaggcggaagctgggggtgtgttcagaagcggtgacgcgg
gcgggcccccggaggtagggggggttccggccccacaggcatgggcggcaggggcacgtcttcgccgcgcgcgggcaggggctgg
tgctggctccgaagagcgcttgcgtgcgcgacgacgcgacggttggtgttcctgtatctggcgcctctgagtgaagaccacgggtcccgtg
accttgaacctgaaagagagttcgacagaatcaatctcggcatcgttgacagcggcctggcgcaggatctcctgcacgtcgcccgagttgt
cctggtaggcgatttctgccatgaactgctcgatctcttcctcctggagatctcctcgtccggcgcgctccacggtggccgccaggtcgttgg
agatgcgacccatgagctgcgagaaggcgttgagtccgccctcgttccagacccggctgtagaccacgcccccctcggcgtcgcgggcg
cgcatgaccacctgggccaggttgagctccacgtgtcgcgtgaagacggcgtagttgcgcaggcgctggaaaaggtagttcagggtggt
ggcggtgtgctcggcgacgaagaagtacatgacccagcgccgcaacgtggattcattgatgtcccccaaggcctccaggcgctccatgg
cctcgtagaagtccacggcgaagttgaaaaactgggagttgcgagcggacacggtcaactcctcctccagaagaacggatgagctcggc
gacagtgtcgcgcacctcgcgctcgaaggccacggggggcgcttcttcctcttccacctcttcttccatgattgcttcttcttcttcctcagccg
ggacgggagggggcggcggcgggggaggggcgcggcggcggcggcggcgcaccgggaggcggtcgatgaagcgctcgatcatct
ccccccgcatgcggcgcatggtctcggtgacggcgcggccgttctcccgggggcgcagctcgaagacgccgcctctcatttcgccgcgg
ggcgggcggccgtgaggtagcgagacggcgctgactatgcatcttaacaattgctgtgtaggtacgccgccaagggacctgattgagtcc
agatccaccggatccgaaaacctttggaggaaagcgtctatccagtcgcagtcgcaaggtaggctgagcaccgtggcgggcgggggcg
ggtcgggagagttcctggcggagatgctgctgatgatgtaattaaagtaggcggtcttgagaaggcggatggtggacaggagcaccatgt
ctttgggtccggcctgttggatgcggaggcggtcggccatgccccaggcctcgttctgacaccggcgcaggtatttgtagtaatcttgcatg
agtctttccaccggcacttcttctccttcctcttcttcatctcgccggtggtttctcgcgccgcccatgcgcgtgaccccaaagcccctgagcgg
ctgcagcagggccaggtcggcgaccacgcgctcggccaagatggcctgctgtacctgagtgagggtcctctcgaagtcatccatgtccac
gaagcggtggtaggcacccgtgttgatggtgtaggtgcagttggccatgacggaccagttgacggtctggtgtcccggctgcgagagctc
cgtgtaccgcaggcgcgagaaggcgcgggaatcgaacacgtagtcgttgcaagtccgcaccagatactggtagcccaccaggaagtgc
ggcggaggttggcgatagaggggccagcgctgggtggcgggggcgccgggcgccaggtcttccagcatgaggcggtggtatccgtag
atgtacctggacatccaggtgatgcctgcggcggtggtggtggcgcgcgcgtagtcgcggacccggttccagatgtttcgcaggggcga
gaagtgttccatggtcggcacgctctggccggtgaggcgcgcgcagtcgttgacgctctatacacacacaaaaacgaaagcgtttacagg
gctttcgttctgtagcctggaggaaagtaaatgggttgggttgcggtgtgccccggttcgagaccaagctgagctcagccggctgaagccg
cagctaacgtggtattggcagtcccgtctcgacccaggccctgtatcctccaggatacggtcgagagcccttttgctttcttggccaagcgcc
cgtggcgcgatctgggatagatggtcgcgatgagaggacaaaagcggctcgcttccgtagtctggagaaacaatcgccagggttgcgttg
cggcgtaccccggttcgagcccctatggcggcttggatcggccggaaccgcggctaacgtgggctgtggcagccccgtcctcaggaccc
cgccagccgacttctccagttacgggagcgagccccttttgtttttttattttttagatgcatcccgtgctgcggcagatgcgcccctcgccccg
gcccgatcagcagcagcaacagcaggcatgcagacccccctctcctctccccgccccggtcaccacggccgcggcggccgtgtccggt
gcggggggcgcgctggagtcagatgagccaccgcggcggcgacctaggcagtatctggacttggaagagggcgagggactggcgcg
gctgggggcgagctctccagagcgccacccgcgggtgcagttgaaaagggacgcgcgtgaggcgtacctgccgcggcaaaacctgttt
cgcgaccgcgggggcgaggagcccgaggagatgcgggactgcaggttccaagcggggcgcgagctgcgccgcggcttggacagac
agcgcctgctgcgcgaggaggactttgagcccgacacgcagacgggcatcagccccgcgcgcgcgcacgtggccgcggccgacctg
gtgaccgcctacgagcagacggtgaaccaggagcgcaacttccaaaaaagcttcaacaaccacgtgcgcacgctggtggcgcgcgagg
aggtgaccctgggtctcatgcatctgtgggacctggtggaggcgatcgtgcagaaccccagcagcaagcccctgaccgcgcagctgttcc
tggtggtgcagcacagcagggacaacgaggccttcagggaggcgctgctgaacatcaccgagccggaggggcgctggctcctggacct
gataaacatcctgcagagcatagtggtgcaggagcgcagcctgagcctggccgagaaggtggcggccattaactattctatgctgagcct
gggcaagttctacgctcgcaagatctacaagaccccctacgtgcccatagacaaggaggtgaagatagacagcttctacatgcgcatggc
gctgaaggtgctaaccctgagcgacgacctgggagtgtaccgcaacgagcgcatccacaaggccgtgagcgccagccggcggcgcga
gctgagcgaccgcgaactgatgcacagtctgcagcgcgcgctgaccggcgcgggcgagggcgacagggaggtcgagtcctactttgac
atgggggccgacctgcactggcagccgagccgccgcgccctggaagcggcgggggcgtacggcggccccctggcggccgatgacg
aggaagaggaggactatgagctagaggagggcgagtacctggaggactgacctggctggtggtgttttggtatagatgcaagatccgaac
gtggcggacccggcggtccgggcggcgctgcagagccagccgtccggcattaactcctctgacgactgggccgcggccatgggtcgca
tcatggccctgaccgcgcgcaaccccgaggccttcaggcagcagcctcaggctaaccggctggcggccatcttggaagcggtagtgccc
gcgcgctccaaccccacccacgagaaggtgctggccatagtcaacgcgctggcggagagcagggccatccgggcagacgaggccgg
actggtgtacgatgcgctgctgcagcgggtggcgcggtacaacagcggcaacgtgcagaccaacctggaccgcctggtgacggacgtg
cgcgaggccgtggcgcagcgcgagcgcttgcatcaggacggcaacctgggctcgctggtggcgctaaacgccttccttagcacccagc
cggccaacgtaccgcgggggcaggaggactacaccaacttcttgagcgcgctgcggctgatggtgaccgaggtccctcagagcgaggt
gtaccagtcggggcccgactacttcttccagaccagcagacagggcttgcaaaccgtgaacctgagccaggctttcaagaacctgcgggg
gctgtggggagtgaaggcgcccaccggcgaccgggctacggtgtccagcctgctaacccccaactcgcgcctgctgctgctgctgatcg
cgcccttcacggacagcgggagcgtctcgcgggagacctatctgggccacctgctgacgctgtaccgcgaggccatcgggcaggcgca
ggtggacgagcacaccttccaggagatcaccagcgtgagccacgcgctggggcaggaggacacgggcagcctgcaggcgaccctga
actacctgctgaccaacaggcggcagaagattcccacgctgcacagcctgacccaggaggaggagcgcatcttgcgctacgtgcagcag
agcgtgagcctgaacctgatgcgcgacggcgtgacgcccagcgtggcgctggacatgaccgcgcgcaacatggaaccgggcatgtac
gcttcccagcggccgttcatcaaccgcctgatggactacttgcatcgggcggcggccgtgaaccccgagtacttcaccaatgccattctgaa
tccccactggatgccccctccgggtttctacaacggggacttcgaggtgcctgaggtcaacgatgggttcctctgggatgacatggatgaca
gtgtgttctcccccaacccgctgcgcgccgcgtctctgcgattgaaggagggctctgacagggaaggaccaaggagtctggcctcctccct
ggctctgggggcggtgggcgccacgggcgcggcggcgcggggcagcagccccttccccagcctggcggactctctgaatagcgggc
gggtgagcaggccccgcttgctaggcgaggaggagtatctgaacaactccctgctgcagcccgtgagggacaaaaacgctcagcggca
gcagtttcccaacaatgggatagagagcctggtggacaagatgtccagatggaagacgtatgcgcaggagtacaaggagtgggaggacc
gccagccgcggcccctgccgccccctagacagcgctggcagcggcgcgcgtccaaccgccgctggaggcaggggcccgaggacgat
gatgactctgcagatgacagcagcgtgttggacctgggcgggagcgggaaccccttttcgcacctgcgcccacgcctgggcaagatgtttt
aaaagagaaaaataaaaactcaccaaggccatggcgacgagcgttggttttttgttcccttccttagtatgcggcgcgcggcgatgttcgag
gaggggcctcccccctcttacgagagcgcgatgggaatttctcctgcggcgcccctgcagcctccctacgtgcctcctcggtacctgcaac
ctacaggggggagaaatagcatctgttactctgagctgcagcccctgtacgataccaccagactgtacctggtggacaacaagtccgcgga
cgtggcctccctgaactaccagaacgaccacagcgattttttgaccacggtgatccaaaacaacgacttcaccccaaccgaggccagtacc
cagaccataaacctggacaacaggtcgaactggggcggcgacctgaagactatcctgcacaccaatatgcccaacgtgaacgagttcatg
ttcaccaactcttttaaggcgcgggtgatggtggcgcgcgagcagggggaggcgaagtacgagtgggtggacttcacgctgcccgaggg
caactactcagagaccatgactctcgacctgatgaacaatgcgatcgtggaacactatctgaaagtgggcaggcagaacggggtgaagga
gagcgatatcggggtcaagtttgacaccagaaacttccgtctgggctgggaccctgtgaccgggctggtcatgccgggggtctacaccaa
cgaggcctttcatcccgatatagtgctcctgcccggctgtggggtggacttcacccagagccggctgagcaacctgctgggcgttcgcaag
cggcaacctttccaggagggtttcaagatcacctatgaggatctggaggggggcaacattcccgcgctccttgatctggacgcctacgagg
agagcttgaaacccgaggagagcgctggcgacagcggcgagagtggcgaggagcaagccggcggcggcggcagcgcgtcggtaga
aaacgaaagtactcccgcagtggcggcggacgctgcggaggtcgagccggaggccatgcagcaggacgcagaggagggcgcgcag
gaggacatgaacaatggggagatcaggggcgacactttcgccacccggggcgaagaaaaagaggcagaggcggcggcggcgacgg
cggaagccgaaaccgaggcagaggcagagcccgagaccgaagttatggaagacatgaatgatggagaacgtaggggtgacacgtttg
ccacccggggcgaagagaaggcggcggaggcagaagccgcggctgaggaggcggctgcggctgcggccaaggctgaggctgcgg
ctgaggctaaggtcgaagccgatgttgcggttgaggctcaggctgaggaggaggcggcggctgaagcagttaaggaaaaggcccaggc
agagcaggaagagaaaaaacctgtcattcaacctctaaaagaagatagcaaaaagcgcagttacaacgtcattgagggcagcacctttacc
caataccgcagctggtacctggcttacaactacggcgacccggtcaagggggtgcgctcgtggaccctgctctgcacgccggacgtcacc
tgcggctccgagcagatgtactggtcgctgccaaacatgatgcaagacccggtgaccttccgttccacgcggcaggttagcaactttccggt
ggtgggcgccgaactgctgccagtacactccaagagtttttacaacgagcaggccgtctactcccagctgatccgccaggccacctctctg
acccacgtgttcaatcgctttcccgagaaccagattttggcgcgcccgccggcccccaccatcaccaccgtcagtgaaaacgttcctgccct
cacagatcacgggacgctaccgctgcgcaacagcatctcaggagtccagcgagtgaccattactgacgccagacgccggacctgcccct
acgtttacaaggccttgggcatagtctcgccgcgcgtcctctccagtcgcactttttaaaacacatccacccacacgctccaaaatcatgtccg
tactcatctcgcccagcaacaacaccggctgggggctgcgcgcacccagcaagatgtttggaggggcaaggaagcgctccgaccagca
ccccgtgcgcgtgcgcggccactaccgcgcgccctggggtgcgcacaagcgcgggcgcacagggcgcaccactgtggatgatgtcatt
gactccgtagtggagcaggcgcgccactacacacccggcgcgccgaccgcctccgccgtgtccaccgtggaccaggcgatcgaaagc
gtggtacagggggcgcggcactatgccaaccttaaaagtcgccgccgccgcgtggcgcgccgccatcgccggagaccccgggctactg
ccgccgcgcgccttaccaaggctctgctcaagcgcgccaggcgaactggccaccgggccgccatgagggccgcacggcgggctgcc
gctgccgcgagcgccgtggccccgcgggcacgaaggcgcgcggccgctgccgccgccgccgccatttccagcttggcctcgacgcgg
cgcggtaacatatactgggtgcgcgactcggtgagcggcacacgtgtgcccgtgcgctttcgccccccacggaattagcacaagacaaca
tacacactgagtctcctgctgttgtgtatcccagcggcgaccgtcagcagcggcgacatgtccaagcgcaaaattaaagaagagatgctcc
aggtcatcgcgccggagatctatgggcccccgaagaaggaggaggaggattacaagccccgcaagctaaagcgggtcaaaaagaaaa
agaaagatgatgacgttgacgaggcggtggagtttgtccgccgcatggcgcccaggcgccctgtgcagtggaagggtcggcgcgtgca
gcgagtcctgcgccccggcaccgcggtggtctttacgcccggcgagcgttccacgcgcactttcaagcgggtgtacgatgaggtgtacgg
cgacgaggatctgttggagcaggccaaccatcgatttggggagtttgcatatgggaaacggcctcgcgagagtctaaaagaggacctgct
ggcgctaccgctggacgagggcaatcccaccccgagtctgaagccggtgaccctgcaacaggtgctgcctttgagcgcgcccagcgag
cagaagcgagggttaaagcgcgagggcggggacctggcacccaccgtgcagttgatggtgcccaagcggcagaagctggaggacgtg
ctggagaaaatgaaagtagagcccgggatccagcccgagatcaaggtccgccctatcaagcaggtggcgcccggcgtgggagtccaga
ccgtggacgttaggattcccacggaggagatggaaacccaaaccgccactccctcttcggcagcaagcgccaccaccggcgccgcttcg
gtagaggtgcagacggacccctggctacccgccgccactatcgccgtcgccgccgccccccgttcgcgcggacgcaagagaaattatcc
agcggccagcgcgcttatgccccagtatgcgctgcatccatccatcgcgcccacccccggctaccgcgggtactcgtaccgcccgcgca
gatcagccggcactcgcggccgccgccgccgtgcgaccacaaccagccgccgccgtcgccgccgccgccagccagtgctgaccccc
gtgtctgtaaggaaggtggctcgctcggggagcacgctggtggtgcccagagcgcgctaccaccccagcatcgtttaaagccggtctctgt
atggttcttgcagatatggccctcacttgtcgccttcgcttcccggtgccgggataccgaggaagaactcaccgccgcaggggcatggcgg
gcagcggtctccgcggcggccgtcgccatcgccggcgcgcaaagagcaggcgcatgcgcggcggtgtgttgcccctgctggtcccgct
actcgccgcggcgatcggcgccgtgcccgggatcgcctccgtggccctgcaggcgtcccagaaacattgactcttgcaaccttgcaagct
tgcatttttggaggaaaaaataaaaaagtctagactctcacgctcgcttggtcctgtgactattttgtagaaaaaagatggaagacatcaacttt
gcgtcgctggccccgcgtcacggctcgcgcccgttcatgggagactggacagatatcggcaccagcaatatgagcggtggcgccttcag
ctggggcagtctgtggagcggccttaaaaattttggttccaccattaagaactatggcaacaaagcgtggaacagcagcacgggtcagatg
ctgagagacaagttgaaagagcagaacttccaggagaaggtggcgcagggcctggcctctggcatcagcggggtggtggacatagcta
accaggccgtgcagaaaaagataaacagtcatctggacccccgccctcaggtggaggaaacgcctccagccatggagacggtgtctccc
gagggcaaaggcgaaaagcgcccgcggcccgacagggaagagaccctggtgtcacacaccgaggagccgccctcttacgaggaggc
agtcaaggccggcctgcccaccactcgccccatagctcccatggccaccggtgtggtgggtcacaggcaacacacccccgcaacactag
atctgcccccgccgtccgagccgactcgccagccaaaggcggtgacggtgtccgctccctccacttccgccgccaacagagtgcctctgc
gccgcgctgcgagcggcccccgggcctcgcgagtcagcggcaactggcagagcacactgaacagcatcgtgggcctgggagtgagg
agtgtgaagcgccgccgttgctactgaatgagcaagctagctaacgtgttgtatgtgtgtatgcgtcctatgtcgccgccagaggagctgttg
agccgccggcgccgtctgcactccagcgaatttcaagatggcgaccccatcgatgatgcctcagtggtcgtacatgcacatctcgggccag
gacgcttcggagtacctgagccccgggctggtgcagttcgcccgcgccacagacacctacttcaacatgagtaacaagttcaggaacccc
actgtggcgcccacccacgatgtgaccacggaccggtcgcagcgcctgacgctgcggttcatccccgtggatcgggaggacaccgctta
ctcttacaaggcgcggttcacgctggccgtgggcgacaaccgcgtgctggacatggcctccacttactttgacatccggggggtgctggac
aggggccccacttttaagccctactcgggcactgcctacaaccccctggcccccaagggcgcccccaattcttgtgagtgggaacaagag
gaaaatcaggtggtcgctgcagatgatgaacttgaagatgaagaagcgcaagcacaagaggaagcccctgtgaaaaaaattcatgtatatg
ctcaggcgcctctttctggcgaaaagatttccaaggatggtatccaaataggtactgaagtcgtaggagatacatctaaggacacttttgcag
ataaaacattccaacccgaacctcagataggcgagtctcagtggaacgaggctgatgccacagcagcaggaggtagagttttgaaaaaga
ctacccctatgagaccttgctatggatcctatgccaggcctaccaatgccaacgggggtcaaggaattatggttgccaatgaacaaggagtg
ttggagtctaaagtagaaatgcaatttttctctaacaccacaacccttaatgcgcgggatggaaccggcaatcccgaaccaaaggtggtgttg
tacagcgaagatgtccacttggaatctcccgatactcatctgtcttacaagcccaaaaaggatgatgttaatgccaaaatcatgttgggtcagc
aagccatgcccaacagacccaacctcattggatttagagataatttcattgggcttatgttttacaacagcaccggtaacatgggagtgctggc
gggtcaggcctctcagttgaatgctgtggtggacttgcaggatagaaacacagaactgtcatatcagcttctgcttgattcaattggggataga
accagatacttctccatgtggaaccaggcagtggatagctatgatccagatgtcagaattattgaaaaccatgggactgaggatgaactgcc
caactactgcttccctttgggcggcataggagttactgatacttatcaagggataaaaaataccaatggcaatggtcagtggaccaaagatga
tcagttcgcggaccgcaacgaaataggggtgggaaacaacttcgccatggagatcaacatccaggccaacctttggagaaacttcctctat
gcaaacgtggggctctacctgccagacaagctcaagtacaaccccaccaacgtggacatctctgacaaccccaacacctatgactacatga
acaagcgggtggtggcccctggcctggtggactgctttgtcaatgtgggagccaggtggtccctggactacatggacaacgtcaacccctt
caaccaccaccgcaatgcgggtctgcgctaccgctccatgatcctgggcaacgggcgctatgtgccctttcacatccaggtaccccagaag
ttctttgccatcaagaacctcctgctcctgcccggctcctacacctacgagtggaacttcaggaaggatgtgaacatggtcctacagagctct
ctgggcaatgaccttagggtggatggggccagcatcaagtttgacagcatcaccctctatgctacatttttccccatggcccacaacaccgcc
tccacgcttgaggccatgctgagaaacgacaccaacgaccagtcctttaatgactacctctctggggccaacatgctctacccaatcccagc
caaggccaccaacgtgcccatctccatcccctctcgcaactgggccgcctttagaggctgggcctttacccgccttaagaccaaggagacc
ccctccctgggctcgggttttgatccctactttgtttactcgggatccatcccctacctggatggcaccttctacctcaaccacactttcaagaag
atatccatcatgtatgactcctccgtcagctggccgggcaacgaccgcttgctcacccccaatgagttcgaggtcaagcgcgccgtggacg
gcgagggctacaacgtggcccagtgcaacatgaccaaggactggttcctggtgcagatgctggccaactacaacataggctaccagggct
tttacatcccagagagctacaaggacaggatgtactccttcttcagaaatttccaacccatgagccgacaggtggtggacgagaccaattac
aaggactatcaagccattggcatcacccaccagcacaacaactcgggtttcgtgggctacctggcgcccaccatgcgcgagggtcaggcc
taccccgccaacttcccctaccccttgataggcaagaccgcggtcgacagcgtcacccagaaaaagttcctctgcgaccgcaccctctggc
gcatccccttctctagcaacttcatgtccatgggtgcgctcacggacctgggccaaaacctgctttatgccaactctgcccatgcgctggaca
tgacttttgaggtggaccccatggacgagcccacccttctctatattgtgtttgaagtgttcgacgtggtcagagtgcaccagccgcaccgcg
gtgtcatcgagaccgtgtacctgcgtacgcccttctcagccggcaacgccaccacctaaggagacagcgccgccgccgcctgcatgacg
ggttccaccgagcaagagctcagggccattgccagagacctgggatgcggaccctattttttgggcacctatgacaaacgcttcccgggctt
tatctcccgagacaagctcgcctgcgccattgtcaacacggccgcgcgcgagaccgggggcgtgcactggctggcctttggctgggacc
cgcgctccaaaacttgctacctctttgacccctttggcttctccgatcagcgcctcaggcagatttatgagtttgagtacgaggggctgctgcg
ccgcagcgcgctcgcctcctcgcccgaccgctgcatcacccttgagaagtccaccgaaaccgtgcaggggccccactcggccgcctgc
ggtctcttctgttgcatgtttttgcacgcctttgtgcactggcctcagagtcccatggattgcaaccccaccatgaacttgctaaagggagtgcc
caacgccatgctccagagcccccaggtccagcccaccctgcgccgcaaccaggaacagctttaccgcttcctggagcgccactcccccta
cttccgcagccacagcgcgcgcatccggggggccacctctattgccacttgcaagaaaacatgcaagacggaaaatgatgtacagcatg
cttttaataaatgtaaagactgtgcactttaattatacacgggctctttctggttatttattcaacaccgccgtcgccatttagaaatcgaaagggtt
ctgccgtgcgtcgccgtgcgccacgggcagagacacgttgcgatactggaagcggctcgcccacttgaactcgggcaccaccatgcgg
ggcagtggttcctcggggaagttctcgctccacagggtgcgggtcagctgcagcgcgctcaggaggtcgggagccgagatcttgaagtc
gcagttggggccggaaccctgcgcgcgcgagttgcggtacacggggttgcagcactggaacaccagcagggccggattattcacgctg
gccagcaggctctcgtcgctgatcatgtcgctgtccagatcctccgcgttgctcagggcgaatggggtcatcttgcagacctgcctgcccag
gaaaggcgggagcccaggcttgccgttgcagtcgcagcgcaggggcattagcaggtgcccacggcccgactgcgcctgcgggtacaa
cgcgcgcatgaaggcttcgatctgcctaaaagccacctgggtcttggctccctccgaaaagaacatcccacaggacttgctggagaactgg
ttcgcgggacagctggcatcgtgcaggcagcagcgcgcgtcagtgttggcaatctgcaccacgttgcgaccccaccggtttttcactatctt
ggccttggaagcctgctcctttagcgcgcgctggccgttctcgctggtcacatccatctctatcacctgttccttgttgatcatgtttgtcccgtg
cagacactttaggtcgccctccgtctgggtgcagcggtgctcccacagcgcgcaaccggtgggctcccaattcttgtgggtcacccccgcg
taggcctgcaggtaggcctgcaggaagcgccccatcatggtcataaaggtcttctggctcgtaaaggtcagctgcaggccgcgatgctctt
cgttcagccaggtcttgcagatggcggccagcgcctcggtctgctcgggcagcatcttaaaatttgtcttcaggtcgttatccacgtggtactt
gtccatcatggcacgcgccgcctccatgcccttctcccaggcggacaccatgggcaggcttagggggtttatcacttccagcggcgagga
caccgtactttcgatttcttcttcctccccctcttcccggcgcgcgcccccgctgttgcgcgctcttaccgcctgcaccaaggggtcgtcttcag
gcaagcgccgcaccgagcgcttgccgcccttgacctgcttgatcagtaccggcgggttgctgaagcccaccatggtcagcgccgcctgct
cttcttcgtcttcgctgtctaccactatttctggggaggggcttctccgctctgcggcaaaggcggcggatcgcttcttttttttcttgggagccg
ccgcgatggagtccgccacggcgaccgaggtcgagggcgtggggctgggggtgcgcggtaccagggcctcgtcgccctcggactcttc
ctctgactccaggcggcggcggagtcgcttctttgggggcgcgcgcgtcagcggcggcggagacggggacggggacggggacggga
cgccctccacagggggtggtcttcgcgcagacccgcggccgcgctcgggggtcttctcgcgctggtcttggtcccgactggccattgtatc
ctcctcctcctaggcagagagacataaggagtctatcatgcaagtcgagaaggaggagagcttaaccaccccctcagagaccgccgatgc
gcccgccgtcgccgtcgcccccgctaccgccgacgcgcccgccacaccgagcgacacccccacggacccccccgccgacgcacccc
tgttcgaggaagcggccgtggagcaggacccgggctttgtctcggcagaggaggatttgcaagaggaggagaataaggaggagaagcc
ctcagtgccaaaagatcataaagagcaagacgagcacgacgcagacgcacaccagggtgaagtcgggcggggggacggagggcatg
gcggcgccgactacctagacgaaggaaacgacgtgctcttgaagcacctgcatcgtcagtgcgccatcgtctgcgacgctctgcaggagc
gcagcgaggtgcccctcagcgtggcggaggtcagccgcgcctacgagctcagcctcttttccccccgggtgcccccccgccgccgcga
aaacggcacatgcgagcccaacccgcgcctcaacttctaccccgcctttgtggtgcccgaggtcctggccacctatcacatcttctttcaaaa
ttgcaagatccccatctcgtgccgcgccaaccgtagccgcgccgataagatgctggccctgcgccagggcgaccacatacctgatatcgc
cgctttggaagatgtgccaaagatcttcgagggtctggggcgcaacgagaagcgggcagcaaactctctgcaacaggaaaacagcgaaa
atgagagtcacactggagcgctggtggagctggagggcgacaacgcccgcctggcggtgctcaagcgcagcatcgaggtcacccacttt
gcctaccccgcgctcaacctgccccccaaagtcatgaacgcggtcatggacgggctgatcatgcgccgcggccggcccctcgctccaga
tgcaaacttgcatgaggagaccgaggacggtcagcccgtggtcagcgacgagcagctgacgcgctggctggagagcgcggaccccgc
cgaactggaggagcggcgcaagatgatgatggccgcggtgctggtcaccgtagagctggagtgtctgcagcgcttcttcggtgaccccg
agatgcagagaaaggtcgaggagaccctacactacaccttccgccagggctacgtgcgccaggcttgcaagatctccaacgtggagctc
agcaacctggtgtcctacctgggcatcttgcatgaaaaccgccttgggcagagcgtgctacactccaccctgcgcggggaggcgcgccg
cgactacgtgcgcgactgcgtttacctcttcctctgctacacctggcagacggccatgggggtctggcagcagtgcctggaggagcgcaa
cctcaaggagctggagaagcttctgcagcgcgcgctcaaagacctctggacgggcttcaacgagcgctcggtggccgccgcgctagcc
gacctcatcttccccgagcgcctgctcaaaaccctccagcaggggctgcccgacttcaccagccaaagcatgttgcaaaattttaggaacttt
atcctggagcgttctggcatcctacccgccacctgctgcgccctgcccagcgactttgtccccctcgtgtaccgcgagtgccccccgccgct
gtggggccactgctacctgttccaactggccaactacctgtcctaccacgcggacctcatggaggactccagcggcgaggggctcatgga
gtgccactgccgctgcaacctctgcacgccccaccgctccctggtctgcaacacccaactgctcagcgagagtcagattatcggtaccttcg
agctacagggtccgtcctcctcagacgagaagtccgcggctccggggctaaaactcactccggggctgtggacttccgcctacctgcgca
aatttgtacctgaagactaccacgcccacgaaatcaggttttacgaggaccaatcccgcccgcccaaggcggagctgaccgcctgcgtcat
cacccagggcgagatcctaggccaattgcaagccatccaaaaagcccgccaagagtttttgctgaagaggggtcggggggtgtatctgga
cccccagtcgggtgaggagctcaacccggttcccccgctgccaccgccgcgggaccttgcttcccaggataagcatcgccatggctccca
gaaagaagcagcagcggccgccgctgccgccgccccacatgctggaggaagaggaggaatactgggacagtcaggcagaggaggttt
cggacgaggaggagccggagacggagatggaagagtgggaggaggacagcttagacgaggaggcttccgaagccgaagaggcagg
cgcaacaccgtcaccctcggccgcagccccctcgcaggcgcccccgaagtccgctcccagcatcagcagcaacagcagcgctataacc
tccgctcctccaccgccgcgacccacggccgaccgcagacccaaccgtagatgggacaccaccggaaccggggccggtaagtcctcc
gggagaggcaagcaagcgcagcgccaaggctaccgctcgtggcgcgctcacaagaacgccatagtcgcttgcttgcaagactgcgggg
ggaacatctccttcgcccgccgcttcctgctcttccaccacggtgtggccttcccccgtaacgtcctgcattactaccgtcatctctacagccc
ctactgcggcggcagtgagccagaggcggccagcggcggcggcgcccgtttcggtgcctaggaagacccagggcaagacttcagcca
agaaactcgcggcgaccgcggcgaacgcggtcgcgggggccctgcgcctgacggtgaacgaacccctgtcgacccgcgaactgagg
aaccgaatcttccccactctctatgccatcttccagcagagcagagggcaggatcaggaactgaaagtaaaaaacaggtctctgcgctccct
cacccgcagctgtctgtatcacaagagcgaagaccagcttcggcgcacgctggaggacgctgaggcactcttcagcaaatactgcgcgct
cactcttaaggactagctccgcgcccttctcgaatttaggcgggaacgcctacgtcatcgcagcgccgccgtcatgagcaaggacattccc
acgccatacatgtggagctatcagccgcagatgggactcgcggcgggcgcctcccaagactactccacccgcatgaactggctcagtgc
cggcccacacatgatctcacaggttaatgacatccgcacccatcgaaaccaaatattggtgaagcaggcggcaattaccaccacgccccg
caataatcccaaccccagggagtggcccgcgtccctggtgtatcaggaaattcccggccccaccaccgtactacttccgcgtgattcccag
gccgaagtccaaatgactaactcaggggcacagctcgcgggcggctgtcgtcacagggtgcggcctcctcgccagggtataactcacct
ggagatccgaggcagaggtattcagctcaacgacgagtcggtgagctcctcgctcggtctcagacctgacgggaccttccagatagccgg
agccggccgatcttccttcacgccccgccaggcgtacctgactctgcagagctcgtcctcggcgccgcgctcgggcggcatcgggactct
ccagttcgtgcaggagtttgtgccctcggtctacttcaaccccttctcgggctctcccggtcgctacccggaccagtttatcccgaactttgac
gccgcgagggactcggtggacggctacgactgaatgtcgggtggacccggtgcagagcaacttcgcctgaagcaccttgaccactgccg
ccgccctcagtgctttgcccgctgtcagaccggtgagttccagtacttttccctgcccgactcgcacccggacggcccggcgcacggggtg
cgctttttcatcccgagtcaggtccgctctaccctaatcagggagttcaccgcccgtcccctactggcggagttggaaaaggggccttctatc
ctaaccattgcctgcatttgctctaaccctggattacaccaagatctttgctgtcatttgtgtgctgagtataataaaggctgagatcagaatctac
tcggaccttatccctttcaattgatcataactgtaatcaataaaaaatcacttacttgaaatctgatagcaagactctgtccaattttttcagcaaca
cttccttcccctcctcccaactctggtactctaggcgcctcctagctgcaaacttcctccacagtctgaagggaatgtcagattcctcctcctgtc
cctccgcacccacgatcttcatgttgttacagatgaaacgcgcgagatcgtctgacgagaccttcaaccccgtgtacccctacgataccgag
atcgctccgacttctgtccctttccttacccctccctttgtatcatccgcaggaatgcaagaaaatccagctggggtgctgtccctgcacctgtc
agagccccttaccacccacaatggggccctgactctaaaaatggggggcggcctgaccctggacaaggaagggaatctcacttcccaaa
acatcaccagtgtcgatccccctctcaaaaaaagcaagaacaacatcagccttcagaccgccgcacccctcgccgtcagctccggggccc
taaccctttttgccactccccccctagcggtcagtggcgacaaccttactgtgcagtctcaggcccctcttactttggaagactcaaaactaact
ctggccaccaaaggacccctaactgtgtccgaaggcaaacttgtcctagaaacagagcctcccctgcatgcaagtgacagcagtagcctg
ggccttagcgtcacggccccacttagcattaacaatgacagcctaggactagacatgcaagcgcccatcagctctcgagatggaaaactgg
ctctaacagtggcggcccccctaactgtggccgagggtatcaatgctttggcagtagccacaggtaatggtattggactaaatgaaaccaac
acacacctgcaggcaaaactggtcgcgcccctaggctttgataccaacggcaacattaagctaagcgtcgcaggaggcatgaggctaaac
aataacacactgatactagatgtaaactacccatttgaggctcaaggccaactgagcctaagagtgggctcgggcccactatatgtagattct
agtagtcataacctaaccattagatgccttaggggattgtatgtaacatcttctaacaaccaaaacggtctagaggccaacattaaactaacaa
aaggccttgtgtatgacggaaatgccatagcagttaatgttggcaaagggctggaatacagccctactggcacaacagaaaaacctataca
gactaaaataggtctaggcatggagtatgacactgagggagccatgatgacaaaactaggctctggactaagctttgacaattcaggagcc
attgtggtgggaaacaaaaatgatgacaggcttactttgtggaccacaccggacccatcgcccaactgtcagatttactctgaaaaagatgct
aaactaaccttggtactgactaaatgtggcagtcaggttgtaggcacagtatctattgccgctcttaaaggtagccttgtgccaatcactagtgc
aatcagtgtggttcagatatacctaaggtttgatgaaaatggggtgctgatgagtaactcttcacttaatggcgaatactggaattttagaaacg
gagactcaactaatggcacaccatatacaaacgcagtgggttttatgcctaatctactggcctatcctaaaggtcaaactacaactgcaaaaa
gtaacattgtcagccaggtctacatgaacggggacgatactaaacccatgacatttacaatcaacttcaatggccttagtgaaacaggggata
cccctgtcagtaaatattccatgacattctcatggaggtggccaaatggaagctacatagggcacaattttgtaacaaactcctttactttctcct
acatcgcccaagaataaagaaagcacagagatgcttgtttttgatttcaaaattgtgtgcttttatttattttcaagcttacagtatttccagtagtca
ttagaatagagcttaattaaactgcatgagaacccttccacatagcttaaatactagtggagaagtactcgcctacatgggggtagagtcataa
tcgtgcatcaggatagggcggtggtgctgcagcagcgcgcgaataaactgctgccgccgccgctccgtcctgcaggaatacaacatggc
agtggtctcctcagcgatgattcgcaccgcccgcagcataaggcgccttgtcctccgggcacagcagcgcaccctgatctcacttaaatca
gcacagtaactgcagcacagcaccacaatattgttcaaaatcccacagtgcaaggcgctgtatccaaagctcatggcggggaccacagaa
cccacgtggccatcataccacaagcgcaggtagattaagtggcgacccctcataaacacgctggacataaacattacctcttttggcatgttg
taattcaccacctcccggtaccatataaacctctgattaaacatggcgccatccaccaccatcctaaaccagctggccaaaacctgcccgcc
ggctatacactgcagggaaccgggactggaacaatgacagtggagagcccaggactcgtaaccatggatcatcatgctcgtcatgatatca
atgttggcacaacacaggcacacgtgcatacacttcctcaggattacaagctcctcccgcgttagaaccatatcccagggaacaacccattc
ctgaatcagcgtaaatcccacactgcagggaagacctcgcacgtaactcacgttgtgcattgtcaaagtgttacattcgggcagcagcggat
gatcctccagtatggtagcgcgggtttctgtctcaaaaggaggtagacgatccctactgtacggagtgcgccgagacaaccgagatcgtgtt
ggtcgtagtgtcatgccaaatggaacgccggacgtagtcatatttcctgaagtcttcactctcacagcaccagcactaatcagagtgtgaaga
gggccaagtgccgaacgagtatatataggaattaaaaatgacgtaaatgtgtaaaggtcaaaaaacgcccagaaaaatacacagaccaac
gcccgaaacgaaaacccgcgaaaaaatacccagaagttcctcaacaaccgccacttccgctttcccacgatacgtcacttcctcaaaaata
gcaaactacatttcccacatgtacaaaaccaaaacccctccccttgtcaccgcccacaacttacataatcacaaacgtcaaagcctacgtcac
ccgccccgcctcgccccgcccacctcattatcatattggcctcaatccaaaataaggtatattattgatgatg

### Example 11 Neoantigens Incorporated into NeoGAd20 and NeoMVA are Immunogenic In Vitro

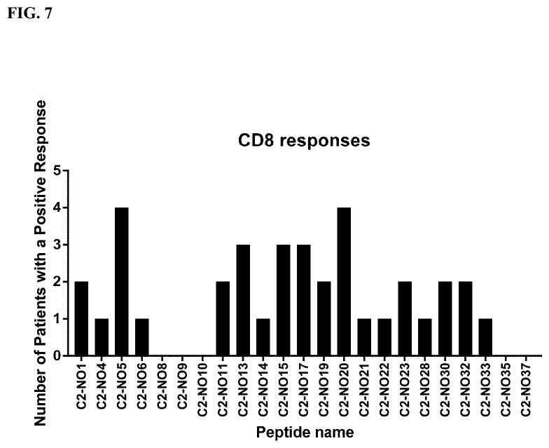

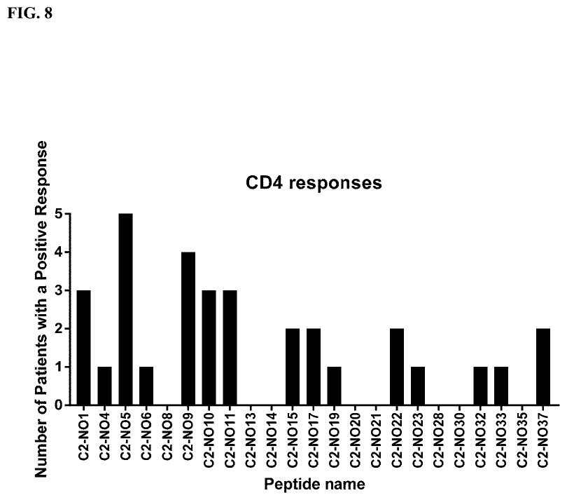

Overlapping 15-mer peptides were designed to span each neoantigen incorporated into NeoGAd20 and NeoMVA to assess their ability to activate T cells using the exogenous autologous normal donor restimulation assay described in Example 1 as pools using TNFα and IFNγ production by CD8⁺ and CD4⁺ T cells as a readout. Table 25 shows the maximum frequency of TNFα⁺IFNγ*CD8⁺ and TNFα⁺IFNγ*CD4⁺ T cells and maximum fold change over negative control for the pool of peptides analyzed, indicating the highest frequency of TNFα⁺IFNγ⁺CD8⁺ and TNFα⁺IFNγ*CD4⁺ T cells and resulting fold change across the normal donors evaluated for the peptide. Table 26 shows the peptide sequences used. FIG. 7 shows the number of patients with a positive CD8+ response for each tested peptide pool for select neoantigens. FIG. 8 shows the number of patients with a positive CD4+ response for each tested peptide pool for select neoantigens.

TABLE 25

| | | | | Maximum |
|---|---|---|---|---|
| | Maximum | | | Frequency |
| | Fold | Maximum | Maximum | of |
| | Change | Frequency of | Fold | TNFα/IFNγ |
| | Over | TNFα/IFNγ | Change | Double |
| Neoantigen ID | Background | Double | Over | Positive |
| (Alternative | CD8 + | Positive | Background | CD4 + |
| name) | cells | CD8 + T cells | CD4 + cells | T cells |
| AS18 (C2-NO1) | 9.17 | 0.110 | 7.51 | 0.053 |
| P87 (C2-NO4) | 9.20 | 0.460 | 7.86 | 0.110 |
| AS55 (C2-NO5) | 1354.17 | 32.500 | 19.38 | 0.310 |
| AS57 (C2-NO6) | 4.67 | 0.056 | 11.00 | 0.110 |
| AS15 (C2-NO11) | 4.36 | 0.061 | 9.17 | 0.110 |
| AS7 (C2-NO13) | 52.60 | 2.630 | 2.50 | 0.045 |
| AS43 (C2-NO15) | 28.75 | 0.460 | 5.67 | 0.040 |
| AS51 (C2-NO17) | 33.13 | 0.530 | 8.75 | 0.140 |
| AS16 (C2-NO19) | 24.17 | 0.290 | 7.78 | 0.140 |
| AS41 (C2-NO20) | 190.60 | 9.530 | 2.20 | 0.022 |
| AS6 (C2-NO22) | 6.50 | 0.078 | 31.67 | 0.380 |
| AS3 (C2-NO23) | 7.92 | 0.095 | 3.86 | 0.054 |
| AS11 | 5.11 | 0.07 | 2.98 | 0.03 |
| AS13 | 1.67 | 0.10 | 1.96 | 0.05 |
| AS47 (C2-NO30) | 4.80 | 0.240 | 2.58 | 0.031 |
| AS8 (C2-NO33) | 54.55 | 0.600 | 4.08 | 0.049 |
| AS19 | 5.87 | 0.63 | 2.59 | 0.1 |
| AS37 | 19.47 | 0.74 | 7.51 | 0.04 |
| AS23 | 3.24 | 0.07 | 5.08 | 0.01 |
| MS1 | 5.56 | 0.1 | 50.19 | 0.39 |
| MS3 | 36.15 | 0.47 | 13.61 | 0.09 |
| MS6 | 4.17 | 0.08 | 111.97 | 0.87 |
| MS8 | 4.44 | 0.14 | 15.60 | 0.40 |
| P82 | 2.92 | 0.09 | 4.44 | 0.17 |
| P16 (C2-NO8) | 2.44 | 0.039 | 2.44 | 0.039 |
| FUS1 (C2-NO9) | 1.94 | 0.031 | 8.33 | 0.100 |
| P22 (C2-NO10) | 1.56 | 0.025 | 18.16 | 0.075 |
| FUS2 (C2-NO14) | 3.13 | 0.05 | 2.14 | 0.03 |
| FUS3 (C2-NO21) | 3.94 | 0.063 | 2.75 | 0.033 |
| FUS6 (C2-NO28) | 3.50 | 0.056 | 2.27 | 0.016 |
| FUS5 (C2-NO32) | 32.50 | 0.390 | 3.43 | 0.048 |
| FUS8 | 1.89 | 0.08 | 7.15 | 0.04 |
| FUS15 (C2- | 1.75 | 0.028 | 2.79 | 0.039 |
| NO35) | | | | |
| P35 | 14.44 | 0.26 | 3.47 | 0.03 |
| FUS19(C2-NO37) | 1.88 | 0.030 | 3.15 | 0.013 |
| FUS7 | 8.89 | 0.16 | 36.13 | 0.04 |
| M84 | 1.39 | 0.03 | 35.84 | 0.31 |
| M86 | 4.22 | 0.08 | 6.18 | 0.05 |
| M10 | 1.89 | 0.09 | 14.14 | 0.1 |
| M12 | 6.67 | 0.12 | 8.94 | 0.05 |
| FR1 | 7.92 | 0.38 | 4.89 | 0.04 |

TABLE 26

| Neoantigen ID | Overlapping Peptide |
|---|---|
| (alternative name) | Sequences* (SEQ ID NO:) |
| AS18 (C2-NO1) | WKFEMSYTVGGPPPH (560) |
| | VGGPPPHVHARPRHW (561) |
| | PPHVHARPRHWKTD (562) |
| P87 (C2-NO4) | YEAGMTLGGKILFFL (563) |
| | GKILFFLFLLLPLSP (564) |
| | FFLFLLLPLSPFSLIF (565) |
| AS55 (C2-NO5) | DGHSYTSKVNCLLLQ (566) |
| | VNCLLLQDGFHGCVS (567) |
| | GFHGCVSITGAAGRR (568) |
| | TGAAGRRNLSIFLFL (569) |
| | LSIFLFLMLCKLEFH (570) |
| | LFLMLCKLEFHAC (571) |
| AS57 (C2-NO6) | TGGKSTCSAPGPQSL (572) |
| | APGPQSLPSTPFSTY (573) |
| | STPFSTYPQWVILIT (574) |
| AS15 (C2-NO11) | VLRFLDLKVRYLHS (269) |
| AS7 (C2-NO13) | DYWAQKEKGSSSFLR (576) |
| | QKEKGSSSFLRPSC (577) |
| AS43 (C2-NO15) | VPFRELKNVSVLEGL (578) |
| | VSVLEGLRQGRLGGP (579) |
| | QGRLGGPCSCHCPRP (580) |
| | SCHCPRPSQARLTPV (581) |
| | QARLTPVDVAGPFLC (582) |
| | VAGPFLCLGDPGLFP (583) |
| | GDPGLFPPVKSSI (584) |
| AS51 (C2-NO17) | GMECTLGQVGAPSPR (585) |
| | VGAPSPRREEDGWRG (586) |
| | EEDGWRGGHSRFKAD (587) |
| | HSRFKADVPAPQGPC (588) |
| | PAPQGPCWGGQPGSA (589) |
| | GGQPGSAPSSAPPEQ (590) |
| | GSAPSSAPPEQSLLD (591) |
| AS16 (C2-NO19) | GNTTLQQLGEASQAP (592) |
| | GEASQAPSGSLIPLR (593) |
| | GSLIPLRLPLLWEVRG (594) |
| AS41 (C2-NO20) | EAFQRAAGEGGPGRG (595) |
| | EGGPGRGGARRGARV (596) |
| | ARRGARVLQSPFCRA (597) |
| | QSPFCRAGAGEWLGH (598) |
| | CRAGAGEWLGHQSLR (599) |
| AS6 (C2-NO22) | DYWAQKEKISIPRTH (600) |
| | QKEKISIPRTHLC (601) |
| AS3 (C2-NO23) | VAMMVPDRQVHYDFG (602) |
| | VPDRQVHYDFGLR (603) |
| AS11 | VPFRELKNQRTAQGA (631) |
| | QRTAQGAPGIHHAAS (632) |
| | GIHHAASPVAANLCD (633) |
| | VAANLCDPARHAQHT (634) |
| | ARHAQHTRIPCGAGQ (635) |
| | IPCGAGQVRAGRGPE (636) |
| | RAGRGPEAGGGVLQP (637) |
| | GGGVLQPQRPAPEKP (638) |
| | RPAPEKPGCPCRRGQ (639) |
| | CPCRRGQPRLHTVKM (640) |
| | RGQPRLHTVKMWRA (641) |
| AS13 | KRSFAVTERII (265) |
| AS47 (C2-NO30) | FKKFDGPCGERGGGR (604) |
| | GERGGGRTARALWAR (605) |
| | ARALWARGDSVLTPA (606) |
| | DSVLTPALDPQTPVR (607) |
| | DPQTPVRAPSLTRAA (608) |
| | PVRAPSLTRAAAAV (609) |
| AS8 (C2-NO33) | LVLGVLSGHSGSRL (255) |
| AS19 | QWQHYHRSGEAAGTP (710) |
| | GEAAGTPLWRPTRN (711) |
| AS37 | CHLFLQPQVGTPPPH (642) |
| | VGTPPPHTASARAPS (643) |
| | ASARAPSGPPHPHES (644) |
| | PPHPHESCPAGRRPA (645) |
| | PAGRRPARAAQTCAR (646) |
| | AAQTCARRQHGLPGC (647) |
| | QHGLPGCEEAGTARV (648) |
| | EAGTARVPSLHLHLH (649) |
| | SLHLHLHQAALGAGR (650) |
| | AALGAGRGRGWGEAC (651) |
| | RGWGEACAQVPPSRG (652) |
| AS23 | KIQNKNCPD (285) |
| MS1 | HYKLIQQPISLFSIT (653) |
| | ISLFSITDRLHKTFS (654) |
| | RLHKTFSQLPSVHLC (655) |
| | LPSVHLCSITFQWGH (656) |
| | ITFQWGHPPIFCSTN (657) |
| | PIFCSTNDICVTANF (658) |
| | ICVTANFCISVTFLK (659) |
| | ISVTFLKPCFLLHEA (660) |
| | CFLLHEASASQ (661) |
| MS3 | RTALTHNQDFSIYRL (662) |
| | DFSIYRLCCKRGSLC (663) |
| | CKRGSLCHASQARSP (664) |
| | ASQARSPAFPKPVRP (665) |
| | FPKPVRPLPAPITRI (666) |
| | PAPITRITPQLGGQS (667) |
| | PQLGGQSDSSQPLLT (668) |
| | SSQPLLTTGRPQGWQ (669) |
| | GRPQGWQDQALRHTQ (670) |
| | QALRHTQQASPASCA (671) |
| | ASPASCATITIPIHS (672) |
| | ITIPIHSAALGDHSG (673) |
| | ALGDHSGDPGPAWDT (674) |
| | PGPAWDTCPPLPLTT (675) |
| | PPLPLTTLIPRAPPP (676) |
| | IPRAPPPYGDSTARS (677) |
| | GDSTARSWPSRCGPLG (678) |
| MS6 | YAYKDFLWCFPFSLV (679) |
| | CFPFSLVFLQEIQIC (680) |
| | LQEIQICCHVSCLCC (681) |
| | HVSCLCCICCSTRIC (682) |
| | CCSTRICLGCLLELF (683) |
| | GCLLELFLSRALRAL (684) |
| | SRALRALHVLWNGFQ (685) |
| | VLWNGFQLHCQ (686) |
| MS8 | TMPAILKLQKNCLLSL (444) |
| P82 | YEAGMTLGEKFRVGN (687) |
| | EKFRVGNCKHLKMTRP (688) |
| P16 (C2-NO8) | GVPGDSTRRAVRRMN (611) |
| | DSTRRAVRRMNTF (612) |
| FUS1 (C2-NO9) | CGASACDVSLIAMDSA (211) |
| P22 (C2-NO10) | SLYHREKQLIAMDSAI (349) |
| FUS2 | TEYNQKLQVNQFSESK (712) |
| FUS3 (C2-NO21) | TEISCCTLSSEENEY (615) |
| | SSEENEYLPRPEWQLQ (616) |
| FUS6 (C2-NO28) | CEERGAAGSLISCE (221) |
| FUSS (C2-NO32) | NSKMALNSEALSVVSE (219) |
| FUS8 | WGMELAASRRFSWDH (689) |
| | RRFSWDHHSAGGPPR (690) |
| | SAGGPPRVPSVRSGA (691) |
| | PSVRSGAAQVQPKDP (692) |
| | QVQPKDPLPLRTLAG (693) |
| | PLRTLAGCLARTAHL (694) |
| | LARTAHLRPGAESLP (695) |
| | PGAESLPQPQLHCT (696) |
| FUS15 (C2-NO35) | HVVGYGHLDTSGSSS (619) |
| | YGHLDTSGSSSSSSWP (620) |
| P35 | NSKMALNSLNSIDDA (697) |
| | LNSIDDAQLTRIAPP (698) |
| | LTRIAPPRSHCCFWE (699) |
| | APPRSHCCFWEVNAP (700) |
| FUS19 (C2-NO37) | KMHFSLKEHPPPPCPP (235) |
| FUS7 | LWFQSSELSPTGAPW (701) |
| | SPTGAPWPSRRPTWR (702) |
| | SRRPTWRGTTVSPRT (703) |
| | TTVSPRTATSSARTC (704) |
| | TSSARTCCGTKWPSS (705) |
| | GTKWPSSQEAALGLG (706) |
| | EAALGLGSGLLRFSC(707) |
| | GLLRFSCGTAAIR (708) |
| M84 | IARELHQFAFDLLIKSH (167) |
| M86 | QPDSFAALHSSLNELGE (171) |
| M10 | FVQGKDWGLKKFIRRDF (19) |
| M12 | FVQGKDWGVKKFIRRDF (23) |
| FR1 | QNLQNGGGSRSSATL (709) |
| | SRSSATLPGRRRRRW (575) |
| | GRRRRRWLRRRRQPI (610) |
| | RRRRQPISVAPAGPP (613) |
| | VAPAGPPRRPNQKPN (614) |
| | RPNQKPNPPGGARCV (617) |
| | PGGARCVIMRPTWPG (618) |
| | MRPTWPGTSAFT (621) |

### Example 12 Neoantigens Incorporated into NeoGAd2 and NeoMVA are Immunogenic when Expressed Endogenously In Vitro

For three of the neoantigens, an Ad5 vector was designed to transduce normal Dendritic cells with the neoantigens. This assay assessed the ability of the endogenously expressed and presented neoantigens to activate autologous T cells following overlapping 15-mer peptide pools restimulation using the endogenous autologous normal donor restimulation assay described in Example 1 utilizing TNFα and IFNγ production by CD8⁺ and CD4⁺ T cells as a readout. Table 27 shows the maximum frequency of TNFα⁺IFNγ⁺CD8⁺ and TNFα⁺IFNγ⁺CD4⁺ T cells and maximum fold change over negative control for the pool of peptides analyzed, indicating the highest frequency of TNFα⁺IFNγ⁺CD8⁺ and TNFα⁺IFNγ⁺CD4⁺ T cells and resulting fold change across the normal donors evaluated for the peptide. Sixteen donors were used to assess endogenous immunogenicity.

TABLE 27

| | | | Maximum | | Maximum |
|---|---|---|---|---|---|
| | | Maximum | Frequency of | Maximum | Frequency of |
| | | Fold Change | TNFα/IFNγ | Fold Change | TNFα/IFNγ |
| | Overlapping Peptide | Over | Double | Over | Double |
| Neoantigen | Sequences* (SEQ ID | Background | Positive | Background | Positive |
| ID | NO:) | CD8+ cells | CD8+ T cells | CD4+ cells | CD4+ T cells |
| AS18 | WKFEMSYTVGGPPPH (560) | 4.09 | 0.36 | 1.90 | 0.046 |
| (C2-NO1) | VGGPPPHVHARPRHW (561) | | | | |
| | PPHVHARPRHWKTD (562) | | | | |
| P87 | YEAGMTLGGKILFFL (563) | 2.47 | 0.39 | 2.41 | 0.079 |
| (C2-NO4) | GKILFFLFLLLPLSP (564) | | | | |
| | FFLFLLLPLSPFSLIF (565) | | | | |
| AS55 | DGHSYTSKVNCLLLQ (566) | 213.88 | 2.05 | 3.50 | 0.063 |
| (C2-NO5) | VNCLLLQDGFHGCVS (567) | | | | |
| | GFHGCVSITGAAGRR (568) | | | | |
| | TGAAGRRNLSIFLFL (569) | | | | |
| | LSIFLFLMLCKLEFH (570) | | | | |
| | LFLMLCKLEFHAC (571) | | | | |

*All generated peptides had NH2 group at N-terminus and —OH group at C-terminus

### Example 13: Neoantigens are Immunogenic In Vitro

Immunogenicity of various additional identified neoantigens was assessed. Overlapping 15-mer peptides were designed to span each neoantigen similarly to what was done in Example 11 to assess their ability to activate T cells using the exogenous autologous normal donor restimulation assay described in Example 1 as pools using TNFα and IFNγ production by CD8⁺ and CD4⁺ T cells as a readout. Table 28 shows the maximum frequency of TNFα⁺IFNγ⁺CD8⁺ and TNFα⁺IFNγ⁺CD4⁺ T cells and maximum fold change over negative control for the pool of peptides analyzed, indicating the highest frequency of TNFα⁺IFNγ+CD8⁺ and TNFα⁺IFNγ+CD4⁺ T cells and resulting fold change across the normal donors evaluated for the peptide. Table 29 shows the amino acid sequences of the peptides used in the assays for each neoantigen.

TABLE 28

| | | Max. | | Max. |
|---|---|---|---|---|
| | Max. Fold Change | Frequency of | Max. Fold Change | Frequency of |
| | over background | CD8 + | over background | CD4 + |
| Neoantigen ID | CD8 + TNFαINFγ | TNFαINFγ | CD4 + TNFαINFγ | TNFαINFγ |
| (Alternative name) | double positive DP | DP | double positive DP | DP |
| AS1 (Misc1-NO12) | 8.15 | 0.11 | 3.57 | 0.02 |
| AS2 (Misc1-NO13) | 5.06 | 0.09 | 110.68 | 0.86 |
| AS4 (Misc1-NO14) | 5.4 | 0.17 | 37.38 | 1.43 |
| AS5 (Misc1-NO15) | 2.13 | 0.16 | 8.71 | 0.1 |
| AS9 (Misc1-NO16) | 3.81 | 0.12 | 4.18 | 0.16 |
| AS10 (Misc1-NO17) | 4.33 | 0.08 | 3.97 | 0.23 |
| AS12 (Misc1-NO18) | 6.03 | 0.19 | 7.32 | 0.28 |
| AS14 (Misc1-NO19) | 3.81 | 0.12 | 1.49 | 0.06 |
| AS17 (Misc1-NO20) | 2.38 | 0.07 | 3.18 | 0.01 |
| AS20 (Misc1-NO21) | 3.81 | 0.12 | 1.36 | 0.05 |
| AS21 (Misc1-NO22) | 3.81 | 0.12 | 1.7 | 0.07 |
| AS22 (Misc1-NO23) | 2.61 | 0.1 | 7.86 | 0.11 |
| AS32 (Misc1-NO24) | 3.81 | 0.12 | 2.04 | 0.08 |
| AS34 (Misc1-NO26) | 16.25 | 0.26 | 9.48 | 0.01 |
| AS35 (Misc1-NO27) | 11.32 | 0.43 | 60.93 | 0.23 |
| AS36 (Misc2-NO1) | 1544.74 | 58.7 | 129.8 | 0.49 |
| AS40 (Misc2-NO3) | 178.52 | 2.41 | 15.34 | 0.89 |
| AS42 (Misc2-NO4) | 4.65 | 0.69 | 24.58 | 0.59 |
| AS44 (Misc2-NO5) | 4.72 | 0.09 | 293.94 | 1.48 |
| AS45 (Misc2-NO6) | 4.96 | 0.07 | 78.51 | 0.61 |
| AS46 (Misc2-NO7) | 11.6 | 0.87 | 157.98 | 0.29 |
| AS48 (Misc2-NO8) | 9.21 | 0.29 | 13.45 | 0.13 |
| AS49 (Misc2-NO9) | 8.67 | 0.65 | 184.87 | 0.14 |
| AS50 (Misc2-NO10) | 1.6 | 0.12 | 17.22 | 0.07 |
| AS52 (Misc2-NO11) | 6.85 | 0.1 | 184.87 | 0.63 |
| AS53 (Misc2-NO12) | 4.02 | 0.35 | 113.91 | 0.43 |
| AS54 | 88.15 | 8.33 | 17.87 | 0.18 |
| AS55.1 (Misc2-NO14) | 25.26 | 1.47 | 20.66 | 0.08 |
| AS56 | 6.35 | 0.6 | 128.76 | 0.18 |
| AS58 (Misc2-NO16) | 4.54 | 0.6 | 6.27 | 0.24 |
| AS59 (Misc2-NO17) | 4.22 | 0.08 | 59.58 | 0.3 |
| FUS9 (Misc1-NO2) | 1.82 | 0.07 | 203.97 | 0.77 |
| FUS10 (Misc1-NO3) | 2.42 | 0.09 | 10.86 | 0.04 |
| FUS11 (Misc1-NO4) | 2.63 | 0.1 | 28.57 | 0.16 |
| FUS18 | 42.33 | 0.91 | 31.76 | 0.04 |
| FUS23 (Misc1-NO6) | 11.05 | 0.62 | 12.77 | 0.12 |
| FUS24 (Misc1-NO7) | 8.53 | 0.64 | 4.4 | 0.05 |
| MS2 (Excl-NO6) | 36.92 | 0.48 | 15.12 | 0.1 |
| MS4 (Excl-NO8) | 24.13 | 0.76 | 367.35 | 2.43 |
| MS5 (Excl-NO9) | 32.89 | 1.95 | 126.05 | 0.2 |
| MS7 (Excl-NO11) | 6.67 | 0.12 | 10.52 | 0.09 |
| MS9 (Excl-NO13) | 1.38 | 0.1 | 455.63 | 1.72 |
| MS10 (Excl-NO14) | 3.42 | 0.13 | 74.17 | 0.28 |
| MS11 (Excl-NO15) | 3.81 | 0.12 | 3.4 | 0.13 |
| P97 (Misc1-NO11) | 7.47 | 1.11 | 3.14 | 0.12 |
| P19 (Misc1-NO8) | 4.76 | 0.15 | 6.67 | 0.16 |
| P27 (Misc1-NO9) | 7.87 | 0.59 | 45.38 | 0.05 |
| P37 (Misc1-NO10) | 2.05 | 0.13 | 22.78 | 0.09 |
| P76, P77 (Misc2- | 4.56 | 0.08 | 53.18 | 0.36 |
| NO18) | | | | |

TABLE 29

| Neoantigen ID | |
|---|---|
| (Alterna- | |
| tive name) | Peptide sequences |
| AS1 (Misc1-NO12) | LTFLDFIQVTLRVMS (SEQ ID NO: 377) |
| | VTLRVMSGSQMENGS (SEQ ID NO: 378) |
| | SQMENGSSYFFKPFS (SEQ ID NO: 415) |
| | YFFKPFSWGLGVGLS (SEQ ID NO: 417) |
| AS2 (Misc1-NO13) | FMIGELVGELCCQLT (SEQ ID NO: 418) |
| | ELCCQLTFRLPFLES (SEQ ID NO: 419) |
| | RLPFLESLCQAVVTQ (SEQ ID NO: 420) |
| | CQAVVTQALRFNPSF (SEQ ID NO: 502) |
| | LRFNPSFQEVCIYQD (SEQ ID NO: 518) |
| | EVCIYQDTDLM (SEQ ID NO: 526) |
| AS4 (Misc1-NO14) | WCPLDLRLGSTGCLT (SEQ ID NO: 527) |
| | GSTGCLTCRHHQTSHE (SEQ ID NO: 714) |
| AS5 (Misc1-NO15) | VVGRRHETAPQPLLV (SEQ ID NO: 715) |
| | APQPLLVPDRAGGEG (SEQ ID NO: 716) |
| | DRAGGEGGA (SEQ ID NO: 717) |
| AS9 (Misc1-NO16) | PVPTATPGVRSVTSP (SEQ ID NO: 718) |
| | VRSVTSPQGLGLFLK (SEQ ID NO: 719) |
| | GLGLFLKFI (SEQ ID NO: 720) |
| AS10 (Misc1- | KENDVREVCDVYLQM (SEQ ID NO: 721) |
| NO17) | CDVYLQMQIFFHFKF (SEQ ID NO: 722) |
| | IFFHFKFRSYFH (SEQ ID NO: 723) |
| AS12 (Misc1- | FARKMLEKVHRQHLQ (SEQ ID NO: 724) |
| NO18) | VHRQHLQLSHNSQE (SEQ ID NO: 725) |
| AS14 (Misc1- | MFLRKEQQVGPHSFS (SEQ ID NO: 726) |
| NO19) | VGPHSFSML (SEQ ID NO: 727) |
| AS17 (Misc1- | GLNLNTDRPGGYSYS (SEQ ID NO: 728) |
| NO20) | PGGYSYSIWWKNNAK (SEQ ID NO: 729) |
| | WWKNNAKNR (SEQ ID NO: 730) |
| AS20 (Misc1- | KVLNEIDAVVTVPPS (SEQ ID NO: 731) |
| NO21) | VVTVPPSLSTSQIPQ (SEQ ID NO: 732) |
| | STSQIPQGCCIIL (SEQ ID NO: 733) |
| AS21 (Misc1- | ANLKGTLQVRSGQAV (SEQ ID NO: 734) |
| NO22) | VRSGQAVSPR (SEQ ID NO: 735) |
| AS22 (Misc1- | LQAAASGQGKQGVPC (SEQ ID NO: 736) |
| NO23) | GKQGVPCPWGCCAYA (SEQ ID NO: 737) |
| | WGCCAYAESPRALIS (SEQ ID NO: 738) |
| | SPRALISGDAPSQVE (SEQ ID NO: 739) |
| | DAPSQVEREVPGPCL (SEQ ID NO: 740) |
| | EVPGPCLNTHSLSHR (SEQ ID NO: 741) |
| | THSLSHRSPQLPGLP (SEQ ID NO: 742) |
| | PQLPGLPHPKQPSV (SEQ ID NO: 743) |
| AS32 (Misc1- | GEVELSEGGEGQRHL (SEQ ID NO: 744) |
| NO24) | GEGQRHLAFPWACSG (SEQ ID NO: 745) |
| | FPWACSGPGWRGVCC (SEQ ID NO: 746) |
| | GWRGVCCAAVEPA (SEQ ID NO: 980) |
| AS34 (Misc1- | KMRAIQAEGGHGQAC (SEQ ID NO: 747) |
| NO26) | GGHGQACCGGAWGWA (SEQ ID NO: 748) |
| | GGAWGWAPGDGGPQG (SEQ ID NO: 749) |
| | GDGGPQGMLTHTLPT (SEQ ID NO: 750) |
| | LTHTLPTLGFQSAWT (SEQ ID NO: 751) |
| | GFQSAWTWRREDADR (SEQ ID NO: 752) |
| | RREDADRAWRTPKAC (SEQ ID NO: 753) |
| | WRTPKACASRRWSI (SEQ ID NO: 754) |
| AS35 (Misc1- | LLEPFRRGEPGPRGL (SEQ ID NO: 755) |
| NO27) | EPGPRGLLSGSSRGG (SEQ ID NO: 756) |
| | SGSSRGGEGPGRSIE (SEQ ID NO: 757) |
| | GPGRSIEAAPATPLP (SEQ ID NO: 758) |
| | APATPLPCCRKNPCR (SEQ ID NO: 759) |
| | CRKNPCRPQPSRFLP (SEQ ID NO: 760) |
| | QPSRFLPPRVLLVII (SEQ ID NO: 761) |
| | RVLLVIILPKLDCPK (SEQ ID NO: 762) |
| | PKLDCPKLGF (SEQ ID NO: 763) |
| AS36 (Misc2-NO1) | PSGRRTKRLVTLRSG (SEQ ID NO: 764) |
| | LVTLRSGCAIQCWHP (SEQ ID NO: 765) |
| | AIQCWHPRAGPVPSA (SEQ ID NO: 766) |
| | AGPVPSALPHTERPP (SEQ ID NO: 767) |
| | PHTERPPRLVRGAAD (SEQ ID NO: 768) |
| | LVRGAADPRTVTLGR (SEQ ID NO: 769) |
| | RTVTLGRSPAVMPRA (SEQ ID NO: 770) |
| | PAVMPRAPA (SEQ ID NO: 771) |
| AS40 (Misc2-NO3) | DCMLSEEGGQARRGG (SEQ ID NO: 772) |
| | GQARRGGSLCSLAAH (SEQ ID NO: 773) |
| | LCSLAAHTIASAARG (SEQ ID NO: 774) |
| | IASAARGRFLSRLSN (SEQ ID NO: 775) |
| | FLSRLSNFCAVVKAS (SEQ ID NO: 776) |
| | CAVVKASRGAPSCTWE (SEQ ID NO: 777) |
| AS42 (Misc2-NO4) | PEPRRLSPGEPRGRP (SEQ ID NO: 778) |
| | GEPRGRPRKGWGIWG (SEQ ID NO: 779) |
| | KGWGIWGLCGARVGP (SEQ ID NO: 780) |
| | CGARVGPKAWR (SEQ ID NO: 781) |
| AS44 (Misc2-NO5) | FVSLTAIQMASSATP (SEQ ID NO: 782) |
| | MASSATPWGRWPVAT (SEQ ID NO: 783) |
| | GRWPVATPTAACPRR (SEQ ID NO: 784) |
| | TAACPRRRPSSLPTG (SEQ ID NO: 785) |
| | PSSLPTGGDSASKKP (SEQ ID NO: 786) |
| | DSASKKPISRRAPWQ (SEQ ID NO: 787) |
| | SRRAPWQPWACPGRS (SEQ ID NO: 788) |
| | WACPGRSVNSAAPRA (SEQ ID NO: 789) |
| | NSAAPRAWCPPATTP (SEQ ID NO: 790) |
| | CPPATTPRTQSPSRD (SEQ ID NO: 791) |
| | TQSPSRDLRPRCLSS (SEQ ID NO: 792) |
| | RPRCLSSWSS (SEQ ID NO: 793) |
| AS45 (Misc2-NO6) | PVAIKPGTGPPNNSS (SEQ ID NO: 794) |
| | GPPNNSSIHGGSKRS (SEQ ID NO: 795) |
| | HGGSKRSENSYCRDL (SEQ ID NO: 796) |
| | NSYCRDLRGQLRAIC (SEQ ID NO: 797) |
| | GQLRAICCSSYSHDR (SEQ ID NO: 798) |
| | SSYSHDRHTTEERGS (SEQ ID NO: 799) |
| | TTEERGSRGRHVWRI (SEQ ID NO: 800) |
| | GRHVWRIRRLHTSGL (SEQ ID NO: 801) |
| | RLHTSGLPCCCHSGP (SEQ ID NO: 802) |
| | CCCHSGPHPRRLPDI (SEQ ID NO: 803) |
| | PRRLPDILRLVTSTK (SEQ ID NO: 804) |
| | RLVTSTKTDHTNTTE (SEQ ID NO: 805) |
| | DHTNTTEGTLDYL (SEQ ID NO: 806) |
| AS46 (Misc2-NO7) | KWNKNWTATLGALTI (SEQ ID NO: 807) |
| | TLGALTIRGHKLLCH (SEQ ID NO: 808) |
| | GHKLLCHLPHLLSSV (SEQ ID NO: 809) |
| | PHLLSSVQQTCRSSSR (SEQ ID NO: 810) |
| AS48 (Misc2-NO8) | ENASLVFTGSNSPIP (SEQ ID NO: 811) |
| | GSNSPIPACELSSHP (SEQ ID NO: 812) |
| | CELSSHPAHGISPWI (SEQ ID NO: 813) |
| | HGISPWIPSPGNEHF (SEQ ID NO: 814) |
| | SPGNEHFHGIKKQVK (SEQ ID NO: 815) |
| | GIKKQVKAIKVE (SEQ ID NO: 816) |
| AS49 (Misc2-NO9) | RLTQRLVQGWTPMEN (SEQ ID NO: 817) |
| | GWTPMENRWCGRRAG (SEQ ID NO: 818) |
| | WCGRRAGGQPASSST (SEQ ID NO: 819) |
| | QPASSSTRWTTCRAA (SEQ ID NO: 820) |
| | WTTCRAACLLTKWTA (SEQ ID NO: 821) |
| | LLTKWTAGRSQTSIG (SEQ ID NO: 822) |
| AS50 (Misc2- | ENSGNASRWLHVPSS (SEQ ID NO: 823) |
| NO10) | WLHVPSSSDDWLGWK (SEQ ID NO: 824) |
| | DDWLGWKKSSAITSNS (SEQ ID NO: 825) |
| AS52 (Misc2- | KGSVERRSVSLGHPA (SEQ ID NO: 826) |
| NO11) | VSLGHPAEGWAWAER (SEQ ID NO: 827) |
| | GWAWAERSLQPGMTT (SEQ ID NO: 828) |
| | LQPGMTTANTGCLSF (SEQ ID NO: 829) |
| | NTGCLSFHHRGCLLP (SEQ ID NO: 830) |
| | HRGCLLPVLPKLHCG (SEQ ID NO: 831) |
| | LPKLHCGLGGLPLVR (SEQ ID NO: 832) |
| | GGLPLVRAKEIKRVQ (SEQ ID NO: 833) |
| | KEIKRVQRAGESSLP (SEQ ID NO: 834) |
| | AGESSLPVKGLLTVA (SEQ ID NO: 835) |
| | KGLLTVASAVIAVLW (SEQ ID NO: 836) |
| | AVIAVLWGRPSEVTG (SEQ ID NO: 837) |
| | RPSEVTGENEAQHD (SEQ ID NO: 838) |
| AS53 (Misc2- | FGLTTLAGRSSHGTS (SEQ ID NO: 839) |
| NO12) | RSSHGTSGLRAATHT (SEQ ID NO: 840) |
| | LRAATHTKSGDGGQG (SEQ ID NO: 841) |
| | SGDGGQGAARQCEKL (SEQ ID NO: 842) |
| | ARQCEKLLELARATR (SEQ ID NO: 843) |
| | ELARATRPWGRSTSA (SEQ ID NO: 844) |
| | WGRSTSASSRWTHRG (SEQ ID NO: 845) |
| | SRWTHRGYMCPPRCA (SEQ ID NO: 846) |
| | MCPPRCAVACW (SEQ ID NO: 847) |
| AS54 | IIDSDKIMAVCMGCL (SEQ ID NO: 848) |
| | DKIMAVCMGCLLTRH (SEQ ID NO: 849) |
| | AVCMGCLLTRHVQCQ (SEQ ID NO: 850) |
| | GCLLTRHVQCQAMEM (SEQ ID NO: 851) |
| | TRHVQCQAMEMQQ (SEQ ID NO: 852) |
| AS55.1 (Misc2- | DGHSYTSKVNCLLLQ (SEQ ID NO: 566) |
| NO14) | VNCLLLQDGFHGCVS (SEQ ID NO: 567) |
| | GFHGCVSITGAAGRR (SEQ ID NO: 568) |
| | TGAAGRRNLSIFLFL (SEQ ID NO: 569) |
| | LSIFLFLMLCKLEFHA (SEQ ID NO: 853) |
| AS56 | LLNAEDYRCAIHSKE (SEQ ID NO: 854) |
| | CAIHSKEIYLLSPSP (SEQ ID NO: 855) |
| | YLLSPSPHQAMDKFS (SEQ ID NO: 856) |
| | QAMDKFSLCCINCNL (SEQ ID NO: 857) |
| | CCINCNLCLHVFLLL (SEQ ID NO: 858) |
| | LHVFLLLLFFQNKDV (SEQ ID NO: 859) |
| | FFQNKDVWLISNIIL (SEQ ID NO: 860) |
| | LISNIILLWIYGGI (SEQ ID NO: 861) |
| AS58 (Misc2- | VETLENANSFSSGIQ (SEQ ID NO: 862) |
| NO16) | SFSSGIQPLLCSLIG (SEQ ID NO: 863) |
| | LLCSLIGLENPT (SEQ ID NO: 864) |
| AS59 (Misc2- | AGAGTISEGSVLHGQ (SEQ ID NO: 865) |
| NO17) | GSVLHGQRLECDARR (SEQ ID NO: 866) |
| | LECDARRFFGCGTTI (SEQ ID NO: 867) |
| | FGCGTTILAEWEHH (SEQ ID NO: 868) |
| FUS9 (Misc1-NO2) | KEQILAVASLVSSQS (SEQ ID NO: 869) |
| | SLVSSQSIHPSWGQS (SEQ ID NO: 870) |
| | HPSWGQSPLSRI (SEQ ID NO: 871) |
| FUS10 (Misc1- | LELELSEGVCFRLR (SEQ ID NO: 229) |
| NO3) | |
| FUS11 (Misc1- | QQLRIFCAAMASNED (SEQ ID NO: 872) |
| NO4) | AMASNEDFS (SEQ ID NO: 873) |
| FUS18 | DGFSGSLFAVVTRRC (SEQ ID NO: 874) |
| | AVVTRRCYFLKWRTI (SEQ ID NO: 875) |
| | FLKWRTIFPQSLMWL (SEQ ID NO: 876) |
| FUS23 (Misc1- | DLRRVATYCAPLPSS (SEQ ID NO: 877) |
| NO6) | CAPLPSSWRPGTGTT (SEQ ID NO: 878) |
| | RPGTGTTIPPRMRSC (SEQ ID NO: 879) |
| FUS24 (Misc1- | LQERMELLACGAERG (SEQ ID NO: 880) |
| NO7) | ACGAERGAGGWGGGG (SEQ ID NO: 881) |
| | GGWGGGGGGGGGDRR (SEQ ID NO: 882) |
| | GGGGDRRGGGGSAPA (SEQ ID NO: 883) |
| | GGGSAPALADFAGGRG (SEQ ID NO: 884) |
| MS2 (Exc1-NO6) | WTDIVKQSVSTNCIS (SEQ ID NO: 885) |
| | VSTNCISIKKGSYTK (SEQ ID NO: 886) |
| | KKGSYTKLFSLVFLI (SEQ ID NO: 887) |
| | FSLVFLIFCWPLIIQL (SEQ ID NO: 888) |
| MS4 (Exc1-NO8) | LRYGALCNVSRISYF (SEQ ID NO: 889) |
| | VSRISYFSLTNIFNF (SEQ ID NO: 890) |
| | LTNIFNFVIKSLTAI (SEQ ID NO: 891) |
| | IKSLTAIFTVKF (SEQ ID NO: 548) |
| MS5 (Exc1-NO9) | RKERNIRKSESTLRL (SEQ ID NO: 892) |
| | SESTLRLSPFPTPAP (SEQ ID NO: 893) |
| | PFPTPAPSGAPAAAQ (SEQ ID NO: 894) |
| | GAPAAAQGKVVRVPG (SEQ ID NO: 895) |
| | KVVRVPGPAGGLVPR (SEQ ID NO: 896) |
| | AGGLVPRDAGARLLP (SEQ ID NO: 897) |
| | AGARLLPPAGGPGGG (SEQ ID NO: 898) |
| | AGGPGGGAAAGEGRA (SEQ ID NO: 899) |
| | AAGEGRAGRGRFPSI (SEQ ID NO: 900) |
| | RGRFPSITEPRPRDL (SEQ ID NO: 901) |
| | EPRPRDLPPRVATGR (SEQ ID NO: 902) |
| | PRVATGRRAGGRRKG (SEQ ID NO: 903) |
| | AGGRRKGAGQGVRTR (SEQ ID NO: 904) |
| | GQGVRTRPLPASWPG (SEQ ID NO: 905) |
| | LPASWPGGRGPFRKG (SEQ ID NO: 906) |
| | RGPFRKGPRRLPLGS (SEQ ID NO: 907) |
| | RRLPLGSGPPAAGVQ (SEQ ID NO: 908) |
| | PPAAGVQRLRCSHLS (SEQ ID NO: 909) |
| | LRCSHLSRGPRRRRG (SEQ ID NO: 910) |
| | GPRRRRGRVCGRACV (SEQ ID NO: 911) |
| | VCGRACVSPPLPPRP (SEQ ID NO: 912) |
| | PPLPPRPPPVGLSAE (SEQ ID NO: 913) |
| | PVGLSAENLSWLSSG (SEQ ID NO: 914) |
| | LSWLSSGLPRACSWR (SEQ ID NO: 915) |
| | PRACSWREFSPETCA (SEQ ID NO: 916) |
| | FSPETCAFRLSGLDS (SEQ ID NO: 917) |
| | RLSGLDSKLSARVER (SEQ ID NO: 918) |
| | LSARVERDLGALRAP (SEQ ID NO: 919) |
| | LGALRAPGSRAAQGG (SEQ ID NO: 920) |
| | SRAAQGGGRVRGSRS (SEQ ID NO: 921) |
| | RVRGSRSEWKTRPWR (SEQ ID NO: 922) |
| | WKTRPWRPPPAWPLT (SEQ ID NO: 923) |
| | PPAWPLTRAGGPLPK (SEQ ID NO: 924) |
| | AGGPLPKNPFLESCS (SEQ ID NO: 925) |
| | PFLESCSETAQRRRV (SEQ ID NO: 926) |
| | TAQRRRVFSFSTPLS (SEQ ID NO: 927) |
| MS7 (Exc1-NO11) | SINKATITGKKDLEL (SEQ ID NO: 928) |
| | GKKDLELILHVSRKK (SEQ ID NO: 929) |
| | LHVSRKKPFLPRVNI (SEQ ID NO: 930) |
| | FLPRVNITPTPISCC (SEQ ID NO: 931) |
| | PTPISCCNLKMLKKF (SEQ ID NO: 932) |
| | LKMLKKFFLLYIIIS (SEQ ID NO: 933) |
| | LLYIIISIIDLTNCL (SEQ ID NO: 934) |
| | IDLTNCLSCYLEHFY (SEQ ID NO: 935) |
| | CYLEHFYRFTFFTDV (SEQ ID NO: 936) |
| | FTFFTDVHYF (SEQ ID NO: 937) |
| MS9 (Exc1-NO13) | PYYSALSGNSWVPST (SEQ ID NO: 938) |
| | NSWVPSTLESDPFGY (SEQ ID NO: 939) |
| | ESDPFGYVFSPLATR (SEQ ID NO: 940) |
| | FSPLATRPALNDQES (SEQ ID NO: 941) |
| | ALNDQESILWPTLTS (SEQ ID NO: 942) |
| | LWPTLTSVVSCALSC (SEQ ID NO: 943) |
| | VSCALSCPSLNLPEN (SEQ ID NO: 944) |
| | SLNLPENWLTLITGG (SEQ ID NO: 945) |
| | LTLITGGMKGGKKMK (SEQ ID NO: 946) |
| | KGGKKMKFTFRH (SEQ ID NO: 947) |
| MS10 (Exc1-NO14) | GLRNLGNTVRAILLS (SEQ ID NO: 948) |
| | VRAILLSFLSKRNVK (SEQ ID NO: 949) |
| | LSKRNVKWCWGWGKP (SEQ ID NO: 950) |
| | CWGWGKPTSLGKACG (SEQ ID NO: 951) |
| | SLGKACGRRALKLF (SEQ ID NO: 952) |
| MS11 (Exc1-NO15) | MEAENAGSLHFHEVL (SEQ ID NO: 953) |
| | LHFHEVLKMGHVKF (SEQ ID NO: 954) |
| P97 (Misc1-NO11) | GYLRMQGLMAQRLLLR (SEQ ID NO: 383) |
| P19 (Misc1-NO8) | WTPIPVLTRWPLPHP (SEQ ID NO: 955) |
| | RWPLPHPPPWRRATS (SEQ ID NO: 956) |
| | PWRRATSCRMARSSP (SEQ ID NO: 957) |
| | RMARSSPSATSGSSV (SEQ ID NO: 958) |
| | ATSGSSVRRRCSSLP (SEQ ID NO: 959) |
| | RRCSSLPSWVWNLAA (SEQ ID NO: 960) |
| | WVWNLAASTRPRSTPS (SEQ ID NO: 961) |
| P27 (Misc1-NO9) | LHPQRETFTPRWSGA (SEQ ID NO: 962) |
| | TPRWSGANYWKLAFP (SEQ ID NO: 963) |
| | YWKLAFPVGAEGTFP (SEQ ID NO: 964) |
| | GAEGTFPAAATQRGV (SEQ ID NO: 965) |
| | AATQRGVVRPA (SEQ ID NO: 966) |
| P37 (Misc1-NO10) | MAGGVLRRLLCREPD (SEQ ID NO: 967) |
| | LLCREPDRDGDKGAS (SEQ ID NO: 968) |
| | DGDKGASREETVVPL (SEQ ID NO: 969) |
| | EETVVPLHIGDPVVL (SEQ ID NO: 970) |
| | IGDPVVLPGIGQCYS (SEQ ID NO: 971) |
| | GIGQCYSALF (SEQ ID NO: 972) |
| P76, P77 (Misc2- | VFFKRAAEGFFRMNK (SEQ ID NO: 973) |
| NO18) | GFFRMNKLKESSDTN (SEQ ID NO: 974) |
| | KESSDTNPKPYCMAA (SEQ ID NO: 975) |
| | KPYCMAAPMGLTENN (SEQ ID NO: 976) |
| | MGLTENNRNRKKSYR (SEQ ID NO: 977) |
| | NRKKSYRETNLKAVS (SEQ ID NO: 978) |
| | TNLKAVSWPLNHT (SEQ ID NO: 979) |

### Diagnostic Assays

### Step 1: Neo-Antigen Burden Estimation

Samples for qPCR analysis—Radical prostatectomy specimens were collected from men diagnosed with prostate adenocarcinoma (PCa). Pathology was reviewed internally to select specimens with greater than or equal to 50% tumor content. Three to five, 5 micron rolls were cut for RNA extraction. Healthy Donor (HD) tissue RNA from ten different body organs (Takara Bio.) and peripheral blood mononuclear cells (PBMCs) from seven healthy donors were used as Normal controls.

RNA extraction, cDNA synthesis, and pre-amplification—RNA was extracted from paraffin rolls using RNAstorm™ kit (Cell Data Sciences) following manufacturer's instructions. Purified RNA was eluted in 30 μl RNase-free water. RNA from PBMCs were extracted using Qiagen's RNeasy mini kit following kit protocol. cDNA was synthesized from 200 ng of PCa sample total RNA and 100 ng of HD tissue and PBMCs RNA using the High Capacity cDNA reverse transcription kit with RNase inhibitor from Applied Biosystems, Foster City, Calif., following the manufacturer's protocol. HD tissue and PBMC cDNA samples were diluted 1:5 with nuclease free water. cDNA samples were then pre-amplified with selected gene panel (SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, and 223) for 14 cycles using AppliedBiosystemsTaqMan™ PreAmp MasterMix per manufacturers protocol. RPL19 gene was used as endogenous control while AR and ARv7 were used as controls for high and low expression of target genes.

Quantitative PCR using BioMark™ 96.96 Dynamic Arrays—Pre-amplified cDNA was diluted 1 to 20 with nuclease free water. Gene expression analysis of diluted preamplified product was performed on Fluidigm's BioMark™ Real-Time PCR system in a 96.96 chip format following the manufacturer's instructions (Fluidigm Corporation, San Francisco, Calif.). Each sample and TaqMan Assay were loaded on the 96.96 chip to give 4 replicate values per sample (2 sample loading+2 TaqMan assay loading).

qPCR data analysis—Raw data were extracted using the BioMark™ real time qPCR analysis software with linear derivative for baseline correction and auto detector for Ct threshold settings. 999 values were converted to 40. Geometric mean Ct of four replicates for each sample were calculated. Ct values greater than 30 were considered as “no amplification.” All samples included in the study were positive for endogenous control, RPL19. The average Ct for RPL19 between the three groups (normal tissue, PBMCs, and prostate adenoma cancer) were in the same range. Relative gene expression for each target and sample was calculated as Ct difference between Target gene and endogenous control gene RPL19. Target genes with gene expression only in PCa samples and not in any of the control HD tissue (except ovary, breast, and prostate) and PBMCs were considered “clean Targets” (i.e. having tumor specific expression) Percent expressed for clean targets were calculated based on number of samples with gene expression×100/total number of samples tested.

Neo-antigen burden—Patient specific neoantigen burden are assessed at the time of patient enrollment into a vaccine clinical trial. Expression profiles generated from step 1 qPCR analysis are used to determine the expression of neoantigens in patient derived biopsies. Additionally, HLA typing is performed on patients to determine the expression of HLA class I antigens. A list of all potential 9 amino acid long peptides from expressed neoantigens are generated, which will be submitted to in-silico methods for HLA class I binding and proteasomal cleavage predictions, such as NetMHCpan 4.0 and NetChop 3.1, to estimate the number of immunogenic 9mer peptides (neoantigen burden) that are likely to be presented by the patient's HLA antigens. A cutoff for the neo-antigen burden is determined based on retrospective analysis of the relationship between patient neoantigen burden and vaccine response in clinical trials. This cutoff will further be used for future patient enrollment strategy. As an illustration, the neoantigen burden for step 1 neoantigens for patients in the Stand Up to Cancer (SU2C) metastatic castration resistant prostate cancer cohort was estimated (data not shown).

The following steps were undertaken to estimate the neoantigen burden in the cohort: Neoantigen protein sequences were run through netChop 3.1 for predictions of cleavage sites using the “Cterm 3.0” method. Cleavage sites that had a predicted score of 0.5 or higher were regarded as legitimate cleavage sites. Peptides with 9 amino acid lengths were generated by choosing amino acid sequences of 9 amino acids upstream and ending at the cleavage site. The resulting peptides were then used to make binding predictions using netMHCpan4.0, and were selected to bind to a HLA allele if their binding rank score was <=2.0. Finally personalized patient neoantigen burden was assessed by i) summing the total number of 9 amino acid peptides that were predicted to bind to a patients HLA Allele profile; and ii) identifying the total number of neo-orfs for a patient that had at least one predicted 9mer binder.

### Step 2: Non-Invasive Monitoring of Disease Burden

Liquid biopsy Samples—Matched blood samples were collected from 20 Healthy Volunteers (HV) and 60 metastatic castrate resistant Prostate Cancer (mCRPC) patients through commercial vendors. Blood from each subject was collected and processed as follows:
PAXgene blood: 2.5 ml blood was collected in PAXgene RNA tube and frozen at −80C freezer until ready for RNA extraction.
Plasma EVs (exosomes): 10 ml blood collected in EDTA tube were processed for plasma within 2 hr of collection. The blood tube was spun for 10 min at 1500×g using swinging bucket rotor. The upper layer of plasma was transferred to a new tube. The plasma tube was centrifuged for 10 min at 3,000×g to remove additional cellular nucleic acids attached to cell debris. The clear supernatant was transferred to a new tube and frozen until ready for EVs enrichment and RNA extraction.

Gene marker selection—Differential gene expression analysis was performed between metastatic and primary prostate cancer tumor samples from multiple studies in Oncomine. Genes were hierarchically clustered into fifteen functional modules and those belonging to the androgen receptor signaling module were selected in the panel. Additionally, genes implicated in the prostate cancer progression and/or response to androgen deprivation therapy were included.

A total of 234 prostate cancer associated gene markers were evaluated. 92 gene markers with more than 30% specificity (100% specificity=no gene expression in healthy donor control samples) were selected for screening metastatic castrate resistant prostate cancer samples (mCRPC).

The lists of biomarkers were further filtered based on their discrepancy in the distinguishing between mCRPC and healthy volunteers. As a result, gene expression derived from 53 genes from plasma exosomes and 55 genes from PAXgene samples (based on their ROC cutoff of 0.55) were used for classification between mCRPC and healthy volunteers using machine learning method. See Tables 33 and 34 below.

TaqMan gene expression assay for ARv7 (also referred to as AR-v3) gene was custom designed while best coverage was selected from Applied Biosystems for the panel of genes and endogenous control GAPDH. The neoantigen ID and primer and probe sequences are listed in Table 30.

TABLE 30
AR-V7 Primers and Probe

| HG19_UID | geneID | ID* | Forward primer | Reverse Primer | MGB Probe |
|---|---|---|---|---|---|
| FUS_794998592_795015535 | ARHGEF38- | FUS2 | TGACAGGACCGAAT | GGCAATGACACCCACTTCCT | ACAAGTGAATCAATT |
| (++) | >ARHGEF38- | | ACAACCAG | | TAGTG |
| | IT1 (++) | | | | |
| FUS_1720900661_1720922933(++) | MSMB- | FUS3 | CTCGGAGTGGCAGA | CTCTGGTCTTGGAAGGTATT | GTTGCACCCTGAGCA |
| | >NCOA4 (−−) | | CTGACAA | CATTC | G |
| | Gene 1 | | | | |
| FUS_2815919412_2818981741(++) | TMPRSS2- | FUS6 | AACTGATAAGGCTT | CGGAGGTGAAAGCGGGT | AACGACTGGTCCTC |
| | ERG(--)_Gene2 | | CACTCACAAC | | CTGC |
| FUS_2495768395_2495769535(++) | INCA1- | FUS8 | GACGCTTGGCACTC | AACCCCCTTCACAATCCCTG | CCTCCGAGACGCTGC |
| | CAMTA2 (−-) | | TGGG | | |
| FUS_2815947912_2818971910(++) | TMPRSS2- | FUS31 | GGAGGGGCAATTCT | GCAGGTCATATTGAACATTC | CAATGGAGTTTAATG |
| | ERG (--)_Gene3 | | TGTCAAC | CAGATAC | AGTTCAA |
| FUS_205623890_205659488(++) | SLC45A3- | | GCTGAAGAAGGAAC | ATCCTGCCCTACACACTGGC | TAGCAATGAGCTGCTT |
| | >ELK4 (--) | FUS1 | TGCCACA | | C |
| | Design1 | | | | |
| FUS_205623890_205661860(++) | SLC45A3- | FUS29 | GCTGAAGAAGGAAC | CTGCTCCCACCTCCACCC | CATAGCAATGAGGGA |
| | >ELK4(--) | | TGCCACA | | GACA |
| | Design 2 | | | | |
| FUS_1720900661_1720923953(++) | MSMB- | FUS27 | ACCTAATGAGGGAG | TCTGGTCTTGGAAGGTATTC | CAACCAGGAGAGCAG |
| | >NCOA4 | | TTCCAGGAG | ATTCT | TG |
| | (++) Gene 2 | | | | |
| FUS_2815919412_2818971910(++) | TMP- | FUS5 | TCAAACAACGACTG | ATTCCAGATACCTATCATTA | CTGATAAGGCTTCTGA |
| | ERG_4 | | GTCCTCACT | CTCGATGC | GTTC |
| FUS_2400843045_2400844698(++) | NME4- | FUS7 | CGCCAGCGACTCCG | AGGTCCGGGCAGAAGAGGT | CAGTGAGCTGTCCCC |
| | DECR2 | | TG | | GA |
| | Gene 1 | | | | |
| FUS_490712493_490755544(++) | D2HGDH- | FUS15 | CGCACGCCAAGCAC | TCCGAGTGCAGGAATCCG | CCACCTTGATACTTCC |
| | GAL3ST2 | | G | | G |
| FUS_2660787519_2660792188(++) | GTF2F1- | FUS19 | ATGGACCAGACAGG | GATCAACGACAAAATGCAC | GTGGGTGCTCCTTGA |
| | PSPN | | GCTCG | TTCTC | |
| CPNE7.89662239, | CPNE7- | AS11 | ACAGTTCGTGCCCT | AGCAACGGGGGAAGCC | AAGAACCAGAGAACA |
| CPNE7.89662332 | 2332_Gene1 | | TCCG | | GCA |
| RECQL4.145738522- | RECQL- | AS37 | CTGCCCACTGCCAC | GCAGGGCAACTTTCATGAG | CCCCAGGTTGGCACC |
| 145738600 | 8600 | | CTCTT | G | |
| RHPN1.144461249- | RHPN1- | AS41 | CCGCCGCGCTATGG | GCAAAAGGGGCTCTGCAAC | GAGGGCCGCTGGTGA |
| 144461484 | 1484 | | A | | G |
| CPNE7.89662012-89662891 | CPNE7- | AS43 | ACAGTTCGTGCCCT | ATGAACAGGGACCCCCAAG | AGAACGTGAGTGTCC |
| | 2891 | | TCCG | | TGG |
| AGRN.976778-976857 | AGRN- | AS47 | CGCCCGCCAGGAGA | TCGAGCCGTGCGCC | CCCTTGTGGTGAGCG |
| | 6857 | | AT | | |
| CPNE7.89643947- | CPNE7- | AS51 | CTGGGGGGCATGGA | CCTTGAATCGGCTGTGGC | TGGGGCAGGTGGGTG |
| APPEQSLLD* | SLLD | | GTG | | C |
| SPOCK1.136443461, | SPOC- | AS64 | TCAGCCATGGTCTG | CACACAGCCATGGAACCCA | CCAAGGTGAATTGTTT |
| SPOCK1.136443559 | 3559_Gene1 | | CGG | | ACTC |
| DNAH8.38866769-38866876-1 | DNAH8- | AS3 | GCGCCAGGAACTAC | GATCCAAGAGTCCTCAATAC | TAGACAGGTTCATTAT |
| | 66876 | | CAGAAA | AGACAG | GACTTT |
| ACSM1.20685193-20685403-1 | ACSM- | AS7 | CAAGTTATGTACTG | GGTCCAAGGTCTTTGACTTA | AGGAGAAGATCAGCA |
| (double skip)_D2 | 5403-Gene2 | | GACTACTGGGCTC | ACACA | TCC |
| ACSM1.20685193-20685403-1 | ACSM1- | AS6 | TTTGCAAGTTATGT | GTAGTCGATAGAGAATGTCT | AGGAGAAGGGATCAT |
| (single skip)_D1 | 5403-Gene1 | | ACTGGACTACTGG | TTGGCC | CTT |
| CACNA1D.53678994- | CACNA1D- | AS8 | GTCAGTCTCGTCAT | CTCCGAAAGTGCTGGGATTA | TGAGCGGGCACAGTG |
| LSGHSGSRL*-1 | GSRL | | CTTTGGGTC | TAG | |
| CPNE7.89595831, | CPNE7- | AS11 | GGACATCGTACAGT | CCGCGTGGTGGATCCC | CTCAAGAACCAGAGA |
| CPNE7.89595924_D1 | 5924-Gene1 | | TCGTGCC | | ACA |
| GRIN3A.101609737- | GRIN3A- | AS13 | GGTGGTTCCCTGTG | TGTCTGTCCTTAGGTCCTCC | TGTCACGGAGAGGAT |
| SFAVTERII*-1 | TERII | | GCAAG | TCC | CA |
| LRRC45.82024340- | LRRC45- | AS15 | TGCCAACACCGTGC | TCCTCTTGCCCTTGGGTG | GGACTTAAAGGTGAG |
| 82024692-1 | 4692 | | TGC | | ATAC |
| LRRC45.82025179- | LRRC45- | AS16 | CCTGAAGGGCAACA | CAGTCCCCTAGCCCCTCACT | CAGCAGCTGGGTGAG |
| 82025378-1 | 5378 | | CCACC | | G |
| ZNF614.52017576, | ZNF61- | AS23 | CCAGATGTACTCTC | ATTGCACTCCAGCCTGGGT | GTCCAGACTGAGTCTC |
| ZNF614.52017697_D1 | 7697-Gene1 | | CAAGTTGGC | | |
| TTLL7.83907686 | TTLL7- | CAS1 | ATCTTAAACCCTCC | TTATGGAGCCTATCAGTGAT | CAACAACCCATATCC |
| | 686-B | | AACCACTACAA | GGAG | CTC |
| TESK1.35608846- | TESK- | CAS2 | TGAGCCAGCCCCAC | CCTCTTGCAACAGAGCCTGT | ACACAATCAGGACTT |
| TATSHPRTV* | RTV-B | | TCAC | AGAT | CT |
| SOAT1.179350277 | SOAT-277-B | CAS3 | GGTGGTCCATGACT | TTTGAATCTCTTGGAGAAAA | CTTTCTCTGGTGTTTT |
| | | | GGCTATATTA | ACTAAAGA | CCTT |
| ZYG11A.52863981- | ZYG-157-B | CAS4 | TTGTGAAGGAAGCC | GACAATTCTTCTGTAACTTT | GACATTTTCAATGGA |
| 52864157 | | | CTCCAC | AAAATAGCAGG | GGTAAC |
| NWD1.16807586- | NWD1-KTDR | AS18 | CAGGCACGCAAGTG | CCTGGGTCTAGCATGAACAT | AGCTACACGGTGGGT |
| RPRHWKTDR*-1 | | | GAAAT | GG | GG |
| AR.67686127-67689555-1 | AR-9555 | AS62 | GTCTTCGGAAATGT | AATGAGGGAGAAGGGGGAG | ACTCTGGGAGGTAAG |
| | | | TATGAAGCAG | AG | ATA |
| KLK3.51362735- | KLK3-RGGV | AS57 | ATGCTGTGTGCTGG | GGTAGGTGGAGAATGGAGT | CACCTGCTCGGCTCCT |
| SLVPWRGGV* | | | ACGCT | GGAG | |
| Androgen receptor | ARv7 | AS61 | GGAAATGTTATGAA | TTTGAGATGCTTGCAATTGC | CTGGGAGAAAAATTC |
| variant 7 | | | GCAGGGATG | C | CGGGT |
| Housekeeping genes | | | | | |
| | | AR | GCTTCTACCAGCTC | GATTAGCAGGTCAAAAGTG | TGCAGCCTATTGCGA |
| | | | ACCAAGCT | AACTGAT | G |
| | | RPL19 | GCGGATTCTCATGG | GGTCAGCCAGGAGCTTCTTG | CCACAAGCTGAAGGC |
| | | | AACACA | | |

ID*= neoantigen ID

RNA extraction, cDNA synthesis and pre-amplification—RNA was extracted using Qiagen kits (Qiagen, Gaithersburg, Md.) as shown in Table 31 following manufacturers protocol.

TABLE 31
Qiagen kits

| | | RNA elution |
|---|---|---|
| Sample type | Qiagen kits | volume (μl) |
| PAXgene blood | PAXgene Blood RNA kit | 80 |
| Plasma EVs | exoRNeasy Serum/plasma maxi kit | 14 |

cDNA was synthesized using 10 μl of total RNA from PAXgene blood and 12 μl from plasma EVs using High capacity cDNA reverse transcription Kit with RNase inhibitor from Applied Biosystems, Foster City, Calif., following manufacturer's instructions. cDNA was pre-amplified with the selected gene panel for 14 cycles using Applied Biosystems TaqMan™ PreAmp MasterMix per manufacturers protocol. PAXgene preamplified cDNA was diluted 1 to 10 while the preamplified plasma EVs cDNA was diluted 1 to 1 with Nuclease free water (Integrated DNA Technologies, Coralville Iowa). Gene expression analysis of diluted preamplified product was performed on Fluidigm's BioMark Real-Time PCR system in a 96.96 chip format following Fluidigm's instructions (Fluidigm Corporation, San Francisco, Calif.). Each sample and TaqMan Assay were loaded on the 96.96 chip to give 4 replicate values per sample (2 sample loading+2 TaqMan assay loading).

Data analysis—Briefly, raw data was extracted from BioMark system using Linear derivative for baseline correction and user detector for CT threshold method. Cycle Threshold (CT) values greater than 999 values were assigned a value of 40. Geometric mean of the 4 replicates was evaluated and an average CT of greater than or equal to 35 was considered to have no detectable expression. Each sample was considered evaluable only when the CT value of endogenous control gene GAPDH was less than 21. Using this threshold, two plasma EVs from the mCRPC cohort were excluded from further analysis. For each gene marker, we calculated the number of samples with detectable expression in mCRPC and HV cohorts.

### Machine Learning Method for Characterization of Disease Sample

Pre-processing of qPCR measurements of prostate cancer associated genes—In this assay, genes with no significantly detectable expression were assigned a PCR Ct value of 33 to 40, genes with high expression (e.g. house-keeping genes such as PTPRC and GAPDH) were assigned a Ct value of less than or equal to 17. Genes with a Ct value larger than 33 were then assigned a value of 33 and genes with a Ct value less than 17 were then assigned a value of 17. Genes with a Ct value of 17 were defined as the most highly expressed genes and genes with a Ct value of 33 were defined as the most lowly expressed genes. The measured qPCR Ct values were then are scaled between 0 and 1 using the following calculation:

A gene with PCR Ct value of 20 is subtracted from 33 (low expression cutoff) and divided by 17 (high expression cutoff) to arrive at a scaled value (e.g., (33-20)/17=0.765).

The scaled value represents the normalized Ct value. A normalized Ct value of 0 represents a lowly expressed gene, while a value of 1 represents a highly expressed gene. These normalized gene expression values were then used in the diagnostic models.

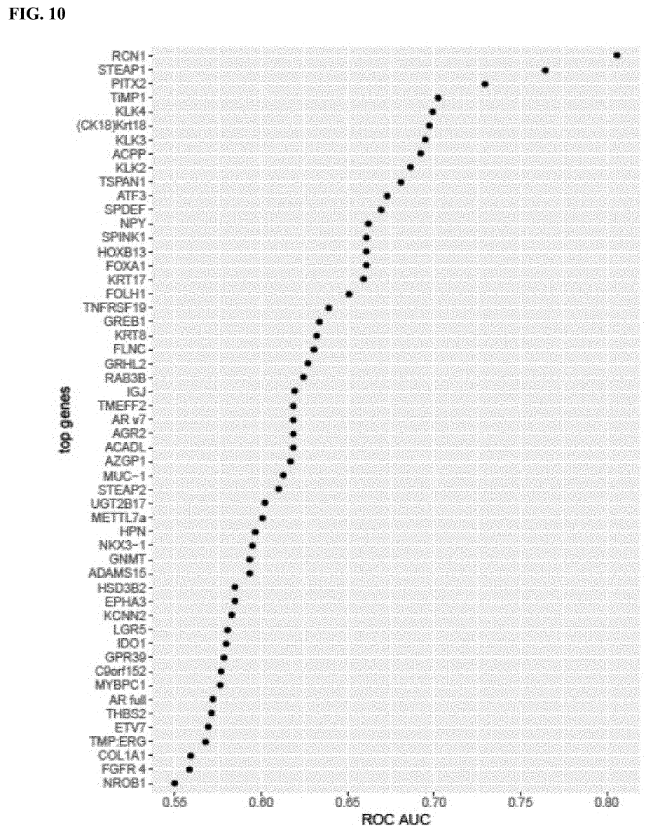

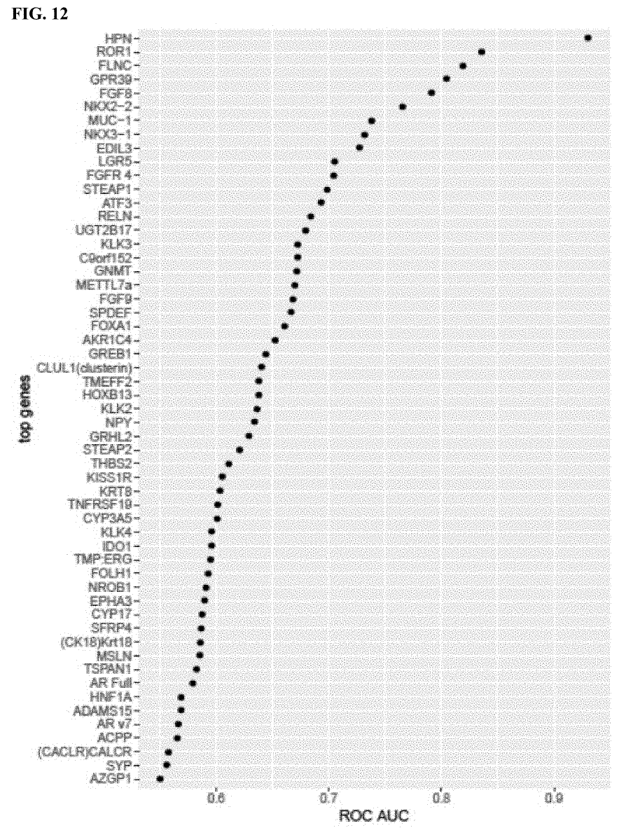

Use of receiver operating characteristic curve (ROC) to select the most informative prostate cancer associated genes—Area Under the Curve (AUC) of the ROC was calculated for each gene to assess its performance in distinguishing between mCRPC samples and healthy samples. The genes were then ranked by the AUC values. The genes with AUC values larger than 0.55 were selected as informative prostate cancer associated genes. See FIGS. 10 and 12. Only those genes were included in the machine learning classification/diagnostic model.

Random forest based diagnostic model and evaluation of its performance—Random forest is a widely used machine learning method. This method was utilized as the classification/diagnostic algorithm based on the panel genes' Ct values. The mCRPC and healthy samples were randomly split into a training dataset and a test dataset, with a ratio of 0.7. In the training step, the algorithm learns the best classification tree structure based on known label of disease and healthy. In the test step, unseen dataset was used to classify the unknown label of the sample based on their normalized PCR Ct values. To achieve robust estimation, the training/test splitting was repeated 200 times, and for each run, the accuracy, sensitivity and specificity of the random forest model in the test dataset were calculated. Considering the following table, the accuracy represents (TP+TN)/(TP+FP+FN+TN), the sensitivity represents TP/(TP+FN), and the specificity represents TN/(TN+FP).

TABLE 32

| | | Truth | |
|---|---|---|---|
| | | mCRPC | Healthy |
| Test | mCRPC | 58 (TP) | 1 (FP) |
| result | Healthy | 2 (FN) | 19 (TN) |

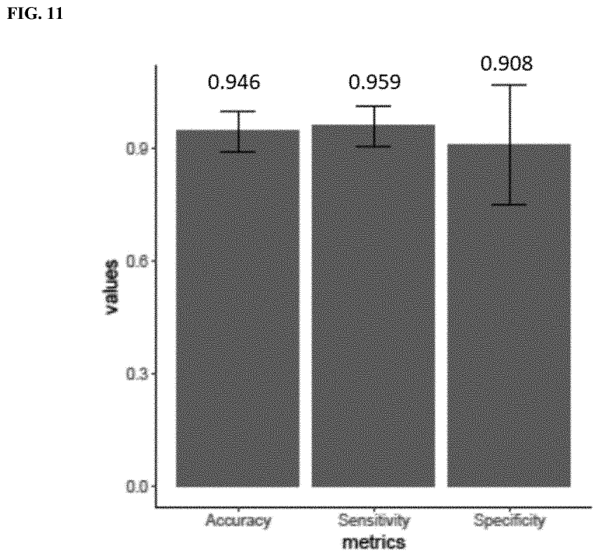

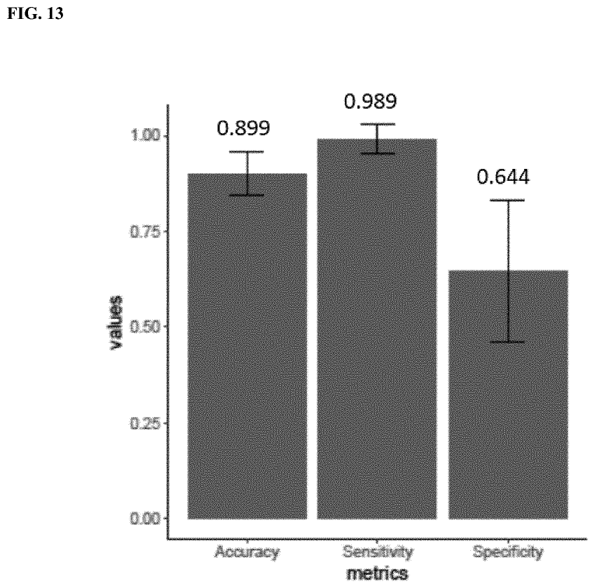

FIG. 11 and FIG. 13 illustrate the mean and standard deviation (error bar) of the accuracy, sensitivity, and specificity for the exosome samples (FIG. 11) and the PAXgene samples (FIG. 13). To evaluate the weights of the genes, all the samples, including mCRPC and healthy, were put in a random forest model to learn their relative importance. Tables 33 and 34 indicate the relative weights of the selected genes from exosomal samples (Table 33) and PAXgene samples (Table 34), where 100 is the most informative gene in this model.

TABLE 33
Relative weights (scaled) of genes based on Exosomal data

| Biomarker | Weight |
|---|---|
| PITX2 | 100 |
| RCN1 | 89.12593 |
| KRT17 | 27.51728 |
| ACPP | 18.77256 |
| ATF3 | 14.70256 |
| ‘NKX3-1’ | 10.86919 |
| ‘(CK18)Krt18’ | 10.64367 |
| TiMP1 | 9.184084 |
| STEAP1 | 8.392075 |
| UGT2B17 | 7.276335 |
| FLNC | 7.005469 |
| METTL7a | 6.846778 |
| KRT8 | 6.659049 |
| IGJ | 6.352049 |
| KLK4 | 6.33976 |
| IDO1 | 6.203417 |
| ETV7 | 3.742933 |
| COL1A1 | 3.404745 |
| ‘AR full’ | 3.050954 |
| LGR5 | 2.514631 |
| AZGP1 | 2.323516 |
| NROB1 | 2.28231 |
| HPN | 2.208863 |
| FOLH1 | 1.868003 |
| TNFRSF19 | 1.811444 |
| TSPAN1 | 1.675128 |
| ‘MUC-1’ | 1.650726 |
| GREB1 | 1.459662 |
| STEAP2 | 1.13545 |
| KCNN2 | 0.921032 |
| RAB3B | 0.887624 |
| ‘FGFR 4’ | 0.857734 |
| NPY | 0.780801 |
| KLK3 | 0.716774 |
| KLK2 | 0.41457 |
| GRHL2 | 0.347752 |
| C9orf152 | 0.302417 |
| SPINK1 | 0.186328 |
| EPHA3 | 0.186303 |
| TMEFF2 | 0.164948 |
| GNMT | 0.164439 |
| ‘TMP:ERG’ | 0.148076 |
| AGR2 | 0.134924 |
| GPR39 | 0.093266 |
| THBS2 | 0.093016 |
| ‘AR v7’ | 0.092698 |
| FOXA1 | 0 |
| ACADL | 0 |
| ADAMS15 | 0 |
| HSD3B2 | 0 |
| MYBPC1 | 0 |

TABLE 34
Relative weights (scaled) of genes based on PAXgene data

| Biomarker | Weight |
|---|---|
| HPN | 100 |
| GPR39 | 94.1898 |
| ROR1 | 79.60198 |
| FLNC | 72.1005 |
| ‘NKX3-1’ | 60.94338 |
| FGF8 | 59.7716 |
| ‘MUC-1’ | 58.30615 |
| EDIL3 | 52.74437 |
| ‘NKX2-2’ | 49.4976 |
| ATF3 | 48.57946 |
| UGT2B17 | 44.08252 |
| ‘FGFR 4’ | 41.47322 |
| GREB1 | 41.29564 |
| LGR5 | 41.01954 |
| FGF9 | 40.03938 |
| RELN | 39.98147 |
| TNFRSF19 | 39.75397 |
| C9orf152 | 36.59141 |
| SYP | 36.456 |
| GNMT | 34.94274 |
| ‘AR Full’ | 30.21543 |
| EPHA3 | 29.92159 |
| METTL7a | 29.89579 |
| STEAP1 | 29.55925 |
| KISS1R | 29.35491 |
| ‘(CACLR)CALCR’ | 29.30495 |
| KRT8 | 28.60391 |
| CYP17 | 28.09351 |
| SPDEF | 26.60297 |
| NPY | 26.55807 |
| MSLN | 26.53083 |
| ‘CLUL1(clusterin)’ | 26.28455 |
| TSPAN1 | 20.31485 |
| FOXA1 | 19.74772 |
| IDO1 | 16.45665 |
| THBS2 | 15.75712 |
| KLK4 | 14.27767 |
| ADAMS15 | 11.59232 |
| CYP3A5 | 10.69601 |
| FOLH1 | 10.25379 |
| TMEFF2 | 7.989344 |
| KLK2 | 7.960658 |
| HOXB13 | 6.969058 |
| ‘(CK18)Krt18’ | 6.759882 |
| SFRP4 | 6.677861 |
| STEAP2 | 6.285243 |
| GRHL2 | 5.215167 |
| ACPP | 4.725696 |
| ‘AR v7’ | 3.702901 |
| NROB1 | 3.615673 |
| ‘TMP:ERG’ | 1.599442 |

### MC38 Tumor Clearance Specific to the GA2 PCaNeoAg.

Mouse colon cancer cell line MC38 was engineered to stably express a protein comprised of 10 prostate neoantigens (Table 35). Multiple MC38 cell lines were identified with a range of protein expression (low, medium, and high) as confirmed by co-expressed mCherry signal. Mice (groups 1-4) were immunized with 1×10⁹ VP of GAd20-PCaNeoAg by intramuscular injection 14 days prior to MC38 cell line implantation. Groups 5-8 did not receive GAd20 immunization (Table 36). MC38 tumor volume (mm³) was recorded through the study for each mouse. Animals immunized with GAd20-PCaNeoAg were shown to have significantly reduced tumor growth (group 3,4) or elimination of tumor (group 2) compared to animals that did not receive GAd20-PCaNeoAg immunization or animals implanted with the parental MC38 cell line that did not express the 10 Prostate Neo antigens. Overall these data indicate that immunization with GAd20-PCaNeoAg leads to MC38 tumor clearance only in tumors that expressing Prostate NeoAg.

TABLE 35

| | | | Lenti |
|---|---|---|---|
| | | | Position |
| | | | to mini- |
| | | | mize |
| | | | Junc- |
| | GAd | | tional |
| Gene | Posi- | | Immuno- |
| ID | tion | Peptide Sequence | genicity |
| SPOC | 12 | DGHSYTSKVNCLLLQDGFHGCVSITGA | 1 |
| | | AGRRNLSIFLFLMLCKLEFHAC (SEQ | |
| | | ID NO: 333) | |
| ZNF614 | 13 | KIQNKNCPD (SEQ ID NO: 285) | 2 |
| AGRN | 14 | FKKFDGPCGERGGGRTARALWARGDS | 9 |
| | | VLTPALDPQTPVRAPSLTRAAAAV | |
| | | (SEQ ID NO: 317) | |
| TTLL7 | 15 | HYKLIQQPISLFSITDRLHKTFSQLPS | 8 |
| | | VHLCSITFQWGHPPIFCSTNDICVTAN | |
| | | FCISVTFLKPCFLLHEASASQ (SEQ | |
| | | ID NO: 437) | |
| RECQL4 | 16 | CHLFLQPQVGTPPPHTASARAPSGPPHP | 3 |
| | | HESCPAGRRPARAAQTCARRQHGLPGC | |
| | | EEAGTARVPSLHLHLHQAALGAGRGR | |
| | | GWGEACAQVPPSRG (SEQ ID NO: | |
| | | 297) | |
| LRRC45 | 17 | VLRFLDLKVRYLHS (SEQ ID NO: | 4 |
| | | 269) | |
| NWD1 | 18 | QWQHYHRSGEAAGTPLWRPTRN (SEQ | 5 |
| | | ID NO: 277) | |
| CPNE7 | 19 | VPFRELKNQRTAQGAPGIHHAASPVAA | 6 |
| | | NLCDPARHAQHTRIPCGAGQVRAGRGP | |
| | | EAGGGVLQPQRPAPEKPGCPCRRGQPR | |
| | | LHTVKMWRA (SEQ ID NO: 261) | |
| DNAH8 | 20 | VAMMVPDRQVHYDFGL (SEQ ID NO: | 7 |
| | | 245) | |
| MSMB- | 21 | GVPGDSTRRAVRRMNTF (SEQ ID | 10 |
| NCOA4-1 | | NO: 343) | |

Subpool 2 NetMHC 4.0 predictions for C57BL/6 MHC Binding:

H-2Kb: 5 predicted strong binding peptides; 11 predicted weak binding peptides

H-2Db: 1 predicted strong binding peptide; 20 predicted weak binding peptides

TABLE 36

| | # | GAd | *MC38 cell line |
|---|---|---|---|
| Group | mice | vaccine | implant |
| 1. Prophylactic GAd vaccine + | 15 | Day 1: 1e9 | Day 15: 5e5 MC38-5 |
| MC38-5 Ag Parental | | vp | Ag parental |
| 2. Prophylactic GAd vaccine + | 15 | Day 1: 1e9 | Day 15: 5e5 MC38-5 |
| MC38-5 Ag ProsNeo Low | | vp | Ag ProsNeo Low |
| 3. Prophylactic GAd vaccine + | 15 | Day 1: 1e9 | Day 15: 5e5 MC38-5 |
| MC38-5 Ag ProsNeo Med | | vp | Ag ProsNeo Med |
| 4. Prophylactic GAd vaccine + | 15 | Day 1: 1e9 | Day 15: 5e5 MC38-5 |
| MC38-5 Ag ProsNeo High | | vp | Ag ProsNeo High |
| 5. MC38-5 Ag Parental | 15 | n/a | Day 15: 5e5 MC38-5 |
| | | | Ag parental |
| 6. MC38-5 Ag ProsNeo Low | 15 | n/a | Day 15: 5e5 MC38-5 |
| | | | Ag ProsNeo Low |
| 7. MC38-5 Ag ProsNeo Med | 15 | n/a | Day 15: 5e5 MC38-5 |
| | | | Ag ProsNeo Med |
| 8. MC38-5 Ag ProsNeo High | 15 | n/a | Day 15: 5e5 MC38-5 |
| | | | Ag ProsNeo High |

SEQ ID NO: 622
CATCATCAATAATATACCTTATTTTGGATTGAGGCCAATATGATAATGAGGTGGGCGGGGCGAGGCGGGGCGGGTGACGTAGGACGCGCGAGTAGG
GTTGGGAGGTGTGGCGGAAGTGTGGCATTTGCAAGTGGGAGGAGCTGACATGCAATCTTCCGTCGCGGAAAATGTGACGTTTTTGATGAGCGCCGCCTACCT
CCGGAAGTGCCAATTTTCGCGCGCTTTTCACCGGATATCGTAGTAATTTTGGGCGGGACCATGTAAGATTTGGCCATTTTCGCGCGAAAAGTGAAACGGGGA
AGTGAAAACTGAATAATAGGGCGTTAGTCATAGCGCGTAATATTTACCGAGGGCCGAGGGACTTTGACCGATTACGTGGAGGACTCGCCCAGGTGTTTTTTA
CGTGAATTTCCGCGTTCCGGGTCAAAGTCTCCGTTTTTATTGTCGCCGTCATCTGACGCGGAGGGTATTTAAACCCGCTGCGCTCCTAAAGAGGCCACTCTT
GAGTGCCAGCGAGAAGAGTTTTCTCCTCCGCTCCGTTTCGGCGATCGAAAAATGAGACATTTAGCCTGCACTCCGGGTCTTTTGTCCGGCCGGGCGGCGTCC
GAGCTTTTGGACGCTTTGCTCAATGAGGTTCTGAGCGATGATTTTCCGTCTACTACCCACTTTAGCCCACCTACTCTTCACGAACTGTACGATCTGGATGTA
CTGGTGGATGTGAACGATCCCAACGAGGAGGCGGTTTCTACGTTTTTTCCCGAGTCTGCGCTTTTGGCTGCCCAGGAGGGATTTGACCTACACACTCCGCCG
CTGCCTATTTTAGAGTCTCCGCTGCCGGAGCCCAGTGGTATACCTTATATGCCTGAACTGCTTCCCGAAGTGGTAGACCTGACCTGCCACGAGCCGGGCTTT
CCGCCCAGCGACGATGAGGGTGAGCCTTTTGCTTTAGACTATGCTGAGATACCTGGGCTCGGTTGCAGGTCTTGTGCATATCATCAGAGGGTTACCGGAGAC
CCCGAGGTTAAGTGTTCGCTGTGCTATATGAGGCTGACCTCTTCCTTTATCTACAGTAAGTTTTTTTGTGTAGGTGGGCTTTTTGGGTAGGTGGGTTTTGTG
GCAGGACAGGTGTAAATGTTGCTTGTGTTTTTTGTACCTGCAGGTCCGGTGTCCGAGCCAGACCCGGAGCCCGACCGCGATCCCGAGCCGGATCCCGAGCCT
CCTCGCAGGCCAAGGAAATTACCTTCCATTTTGTGCAAGCCTAAGACACCTGTGAGGACCAGCGAGGCGGACAGCACTGACTCTGGCACTTCTACCTCTCCT
CCTGAAATTCACCCAGTGGTTCCTCTGGGTATACATAGACCTGTTGCTGTTAGAGTTTGCGGGCGACGCCCTGCAGTAGAGTGCATTGAGGACTTGCTTAAC
GATCCCGAGGGACCTTTGGACTTGAGCATTAAACGCCCTAGGCAATAAACCCCACCTAAGTAATAAACCCCACCTAAGTAATAAACTTTACCGCCCTTGGTT
ATTGAGATGACGCCCAATGTTTGCTTTTGAATGACTTCATGTGTATAATAAAAGTGAGTGTGGTCATAGGTCTCTTGTTTGTCTGGGCGGGGTTTAAGGGTA
TATAAGTTTCTCGGGGCTAAACTTGGTTACACTTGACCCCAATGGAGGCGTGGGGGTGCTTGGAGGAGTTTGCGGACGTGCGCCGTTTGCTGGACGAGAGCT
CTAGCAATACCTATAGTATTTGGAGGTATCTGTGGGGCTCTACTCAGGCCAAGTTGGTCTTCAGAATTAAGCAGGATTACAAGTGCGATTTTGAAGAGCTTT
TTAGTTCCTGTGGTGAGCTTTTGCAATCCTTGAATCTGGGCCACCAGGCTATCTTCCAGGAAAAGGTTCTCTCGACTTTGGATTTTTCCACTCCCGGGCGCA
CCGCCGCTTGTGTGGCTTTTGTGTCTTTTGTGCAAGATAAATGGAGCGGGGAGACCCACCTGAGTCACGGCTACGTGCTGGATTTCATGGCGATGGCTCTTT
GGAGGGCTTACAACAAATGGAAGATTCAGAAGGAACTGTACGGTTCCGCCCTACGTCGTCCACTTCTGCAGCGGCAGGGGCTGATGTTTCCCGACCATCGCC
AGCATCAGAATCTGGAAGACGAGCGAGCGGAGAAGATCAGCTTGAGAGCCGGCCTGGACCCTCCTCAGGAGGAATGAATCTCCCGCAGGTGGTTGAGCTGTT
TCCCGAACTGAGACGGGTCCTGACTATCAGGGAGGATGGTCAGTTTGTGAAGAAGCTGAAGAGGGATCGGGGTGAGGGAGATGATGAGGCGGCTAGCAATTT
AGCTTTTAGTCTGATAACTCGCCACCGACCGGAATGTATTACCTATCAGCAGATTAAGGAGAGTTGTGCCAACGAGCTGGATCTTTTGGGTCAGAAGTATAG
CATAGAACAGCTTACCACTTACTGGCTTCAGCCCGGGGATGATTGGGAAGAGGCGATTAGGGTGTATGCAAAGGTGGCCCTGCGGCCCGATTGCAAGTATAA
GATTACTAAGTTGGTTAATATTAGAAACTGCTGCTATATTTCTGGAAACGGGGCCGAAGTGGAGATAGATACTGAGGACAGGGTGGCTATTAGGTGTTGCAT
GATAAACATGTGGCCCGGGATACTGGGGATGGATGGGGTGATATTTATGAATGTGAGGTTCACGGGCCCCAACTTTAATGGTACGGTGTTCATGGGCAACAC
CAACTTGCTCCTGCATGGTGCGAGTTTCTATGGGTTTAACAACACCTGTATAGAGGCCTGGACCGATGTAAAGGTTCGAGGTTGTTCCTTTTATAGCTGTTG
GAAGGCGGTGGTGTGTCGCCCTAAAAGCAGGGGTTCTGTGAAGAAATGCTTGTTTGAAAGGTGCACCCTAGGTATCCTTTCTGAGGGCAACTCCAGGGTGCG
CCATAATGTGGCTTCGAACTGCGGTTGCTTCATGCAAGTGAAGGGGGTGAGCGTTATCAAGCATAACTCGGTCTGTGGAAACTGCGAGGATCGCGCCTCTCA
GATGCTGACCTGCTTTGATGGCAACTGTCACCTGTTGAAGACCATTCATATAAGCAGTCACCCCAGAAAGGCCTGGCCCGTGTTTGAGCATAACATTCTGAC
CCGCTGTTCCTTGCATCTGGGGGTCAGGAGGGGTATGTTCCTGCCTTACCAGTGTAACTTTAGCCACACTAAAATCCTGCTGGAACCCGAGTGCATGACTAA
GGTCAGCCTGAATGGTGTGTTTGATGTGAGTCTGAAGATTTGGAAGGTGCTGAGGTATGATGAGACCAGGACCAGGTGCCGACCCTGCGAGTGCGGCGGCAA
GCACATGAGAAATCAGCCTGTGATGTTGGATGTGACCGAGGAGCTTAGGCCTGACCATCTGGTGCTGGCCTGCACCAGGGCCGAGTTTGGGTCTAGCGATGA
GGATACCGATTGAGGTGGGTAAGGTGGGCGTGGCTAGCAGGGTGGGCGTGTATAAATTGGGGGTCTAAGGGGTCTCTCTGTTTGTCTTGCAACAGCCGCCGC
CATGAGCGACACCGGCAACAGCTTTGATGGAAGCATCTTTAGTCCCTATCTGACAGTGCGCATGCCTCACTGGGCCGGAGTGCGTCAGAATGTGATGGGTTC
CAACGTGGATGGACGTCCCGTTCTGCCTTCAAATTCGTCTACTATGGCCTACGCGACCGTGGGAGGAACTCCGCTGGACGCCGCGACCTCCGCCGCCGCCTC
CGCCGCCGCCGCGACCGCGCGCAGCATGGCTACGGACCTTTACAGCTCTTTGGTGGCGAGCAGCGCGGCCTCTCGCGCGTCTGCTCGGGATGAGAAACTGAC
TGCTCTGCTGCTTAAACTGGAAGACTTGACCCGGGAGCTGGGTCAACTGACCCAGCAGGTTTCCAGCTTGCGTGAGAGCAGCCTTGCCTCCCCCTAATGGCC
CATAATATAAATAAAAGCCAGTCTGTTTGGATTAAGCAAGTGTATGTTCTTTATTTAACTCTCCGCGCGCGGTAAGCCCGGGACCAGCGGTCTCGGTCGTTT
AGGGTGCGGTGGATTTTTTCCAACACGTGGTACAGGTGGCTCTGGATGTTTAGATACATGGGCATGAGTCCATCCCTGGGGTGGAGGTAGCACCACTGCAGA
GCTTCGTGCTCGGGGGTGGTGTTGTATATGATCCAGTCGTAGCAGGAGCGCTGGGCGTGGTGCTGAAAAATGTCCTTAAGCAAGAGGCTTATAGCTAGGGGG
AGGCCCTTGGTGTAAGTGTTTACAAATCTGCTTAGCTGGGAGGGGTGCATCCGGGGGGATATGATGTGCATCTTGGACTGGATTTTTAGGTTGGCTATGTTC
CCGCCCAGATCCCTTCTGGGATTCATGTTGTGCAGGACCACCAGCACGGTATATCCAGTGCACTTGGGAAATTTATCGTGGAGCTTAGACGGGAATGCATGG
AAGAACTTGGAGACGCCCTTGTGGCCTCCCAGATTTTCCATACATTCGTCCATGATGATGGCAATGGGCCCGTGGGAAGCTGCCTGAGCAAAAACGTTTCTG
GCATCGCTCACATCGTAGTTATGTTCCAGGGTGAGGTCATCATAGGACATCTTTACGAATCGGGGGCGAAGGGTCCCGGACTGGGGGATGATGGTACCCTCG
GGCCCCGGGGCGTAGTTCCCCTCACAGATCTGCATCTCCCAGGCTTTCATTTCAGAGGGAGGGATCATATCCACCTGCGGGGCGATGAAAAAGACAGTTTCT
GGCGCAGGGGAGATTAACTGGGATGAGAGCAGGTTTCTGAGCAGCTGTGACTTTCCACAGCCGGTGGGCCCATATATCACGCCTATCACCGGCTGCAGCTGG
TAGTTAAGAGAGCTGCAGCTGCCGTCCTCCCGGAGCAGGGGGGCCACCTCGTTGAGCATATCCCTGACGTGGATGTTCTCCCTGACCAGTTCCGCCAGAAGG
CGCTCGCCGCCCAGCGAAAGCAGCTCTTGCAAGGAAGCAAAATTTTTCAGCGGTTTCAGGCCATCGGCCGTGGGCATGTTTTTCAGCGTCTGGGTCAGCAGC
TCCAGCCTGTCCCAGAGCTCGGTGATGTGCTCTACGGCATCTCGATCCAGCAGATCTCCTCGTTTCGCGGGTTGGGGCGGCTTTCGCTGTAGGGCACCAGCC
GATGGGCGTCCAGCGGGGCCAGAGTCATGTCCTTCCATGGGCGCAGGGTCCTCGTCAGGGTGGTCTGGGTCACGGTGAAGGGGTGCGCTCCGGGTTGGGCAC
TGGCCAGGGTGCGCTTGAGGCTGGTTCTGCTGGTGCTGAATCGCTGCCGCTCTTCGCCCTGCGCGTCGGCCAGGTAGCATTTGACCATGGTCTCGTAGTCGA
GACCCTCGGCGGCGTGCCCCTTGGCGCGGAGCTTTCCCTTGGAGGTGGCGCCGCACGAGGGGCACTGCAGGCTCTTCAGGGCGTAGAGCTTGGGAGCGAGAA
ACACGGACTCTGGGGAGTAGGCGTCCGCGCCGCAGGCCGAGCAGACCGTCTCGCATTCCACCAGCCAAGTGAGTTCCGGGCGGTCAGGGTCAAAAACCAGGT
TGCCCCCATGCTTTTTGATGCGTTTCTTACCTTGGCTCTCCATGAGGCGGTGTCCCTTCTCGGTGACGAAGAGGCTGTCCGTGTCCCCGTAGACCGACTTCA
GGGGCCTGTCTTCCAGCGGAGTGCCTCTGTCCTCCTCGTAGAGAAACTCTGACCACTCTGAGACGAAGGCCCGCGTCCAGGCCAGGACGAAGGAGGCCACGT
GGGAGGGGTAGCGGTCGTTGTCCACTAGCGGGTCCACCTTCTCCAGGGTGTGCAGGCACATGTCCCCCTCCTCCGCGTCCAGAAAAGTGATTGGCTTGTAGG
TGTAGGACACGTGACCGGGGGTTCCCAACGGGGGGGTATAAAAGGGGGTGGGTGCCCTTTCATCTTCACTCTCTTCCGCATCGCTGTCTGCGAGAGCCAGCT
GCTGGGGTAAGTATTCCCTCTCGAAGGCGGGCATGACCTCAGCGCTCAGGTTGTCAGTTTCTAAAAATGAGGAGGATTTGATGTTCACCTGTCCGGAGGTGA
TACCTTTGAGGGTACCTGGGTCCATCTGGTCAGAAAACACTATTTTTTTGTTATCAAGCTTGGTGGCGAATGACCCGTAGAGGGCGTTGGAGAGCAGCTTGG
CGATGGAGCGCAGGGTCTGGTTTTTGTCGCGGTCGGCTCGCTCCTTGGCCGCGATGTTGAGTTGCACGTACTCGCGGGCCACGCACTTCCACTCGGGGAACA
CGGTGGTGCGCTCGTCTGGGATCAGGCGCACCCTCCAGCCGCGGTTGTGCAGGGTGACCATGTCGACGCTGGTGGCGACCTCACCGCGCAGACGCTCGTTGG
TCCAGCAGAGGCGGCCGCCCTTGCGCGAGCAGAAGGGGGGTAGGGGGTCCAGCTGGTCCTCGTTTGGGGGGTCCGCGTCGATGGTAAAGACCCCGGGGAGCA
GGCGCGGGTCAAAGTAGTCGATCTTGCAAGCTTGCATGTCCAGAGCCCGCTGCCATTCGCGGGCGGCGAGCGCGCGCTCGTAGGGGTTGAGGGGCGGGCCCC
AGGGCATGGGGTGGGTGAGCGCGGAGGCGTACATGCCGCAGATGTCATACACGTACAGGGGTTCCCTGAGGATACCGAGGTAGGTGGGGTAGCAGCGCCCCC
CGCGGATGCTGGCGCGCACGTAGTCATAGAGCTCGTGGGAGGGGGCCAGCATGTTGGGCCCGAGGTTGGTGCGCTGGGGGCGCTCGGCGCGGAAGACGATCT
GCCTGAAGATGGCGTGGGAGTTGGAGGAGATGGTGGGCCGCTGGAAGACGTTGAAGCTTGCTTCTTGCAAGCCCACGGAGTCCCTGACGAAGGAGGCGTAGG
ACTCGCGCAGCTTGTGCACCAGCTCGGCGGTGACCTGGACGTCGAGCGCACAGTAGTCGAGGGTCTCGCGGATGATGTCATACCTATCCTCCCCCTTCTTTT
TCCACAGCTCGCGGTTGAGGACGAACTCTTCGCGGTCTTTCCAGTACTCTTGGAGGGGAAACCCGTCCGTGTCCGAACGGTAAGAGCCTAGCATGTAGAACT
GGTTGACGGCCTGGTAGGGGCAGCAGCCCTTCTCCACGGGCAGCGCGTAGGCCTGCGCCGCCTTGCGGAGGGAGGTGTGGGTGAGGGCGAAAGTGTCCCTGA
CCATGACTTTGAGGTATTGATGTCTGAAGTCTGTGTCATCGCAGCCGCCCTGTTCCCACAGGGTGTAGTCCGTGCGCTTTTTGGAGCGCGGGTTGGGCAGGG
AGAAGGTGAGGTCATTGAAGAGGATCTTCCCCGCTCGAGGCATGAAGTTTCTGGTGATGCGAAAGGGCCCTGGGACCGAGGAGCGGTTGTTGATGACCTGGG
CGGCCAGGACGATCTCGTCAAAGCCGTTTATGTTGTGTCCCACGATGTAGAGCTCCAGGAAGCGGGGCTGGCCCTTGATGGAGGGGAGCTTTTTAAGTTCCT
CGTAGGTAAGCTCCTCGGGCGATTCCAGGCCGTGCTCCTCCAGGGCCCAGTCTTGCAAGTGAGGGTTGGCCGCCAGGAAGGATCGCCAGAGGTCGCGGGCCA
TGAGGGTCTGCAGGCGGTCGCGGAAGGTTCTGAACTGCCGCCCCACGGCCATTTTTTCGGGGGTGATGCAGTAGAAGGTGAGGGGGTCTTTCTCCCAGGGGT
CCCATCTGAGCTCTCGGGCGAGGTCGCGCGCGGCAGCGACCAGAGCCTCGTCGCCCCCCAGTTTCATGACCAGCATGAAGGGCACGAGTTGCTTGCCAAAGG
CTCCCATCCAAGTGTAGGTTTCTACATCGTAGGTGACAAAGAGGCGCTCCGTGCGAGGATGAGAGCCGATTGGGAAGAACTGGATCTCCCGCCACCAGTTGG
AGGATTGGCTGTTGATGTGGTGAAAGTAGAAGTCCCGTCTGCGGGCCGAGCACTCGTGCTGGCTTTTGTAAAAGCGACCGCAGTACTGGCAGCGCTGCACGG
GTTGTATATCTTGCACGAGGTGAACCTGGCGACCTCTGACGAGGAAGCGCAGCGGGAATCTAAGTCCCCCGCCTGGGGTCCCGTGTGGCTGGTGGTCTTTTA
CTTTGGTTGTCTGGCCGCCAGCATCTGTCTCCTGGAGGGCGATGGTGGAACAGACCACCACGCCGCGAGAGCCGCAGGTCCAGATCTCGGCGCTCGGCGGGC
GGAGTTTGATGACGACATCGCGCACATTGGAGCTGTCCATGGTCTCCAGCTCCCGCGGCGGCAGGTCAGCCGGGAGTTCCTGGAGGTTCACCTCGCAGAGAC
GGGTCAAGGCGCGGACAGTGTTGAGATGGTATCTGATTTCAAGGGGCATGTTGGAGGCGGAGTCGATGGCTTGCAGGAGGCCGCAGCCCCGGGGGGCCACGA
TGGTTCCCCGCGGGGCGCGAGGGGAGGCGGAAGCTGGGGGTGTGTTCAGAAGCGGTGACGCGGGCGGGCCCCCGGAGGTAGGGGGGGTTCCGGCCCCACAGG
CATGGGCGGCAGGGGCACGTCTTCGCCGCGCGCGGGCAGGGGCTGGTGCTGGCTCCGAAGAGCGCTTGCGTGCGCGACGACGCGACGGTTGGTGTCCTGTAT
CTGGCGCCTCTGAGTGAAGACCACGGGTCCCGTGACCTTGAACCTGAAAGAGAGTTCGACAGAATCAATCTCGGCATCGTTGACAGCGGCCTGGCGCAGGAT
CTCCTGCACGTCGCCCGAGTTGTCCTGGTAGGCGATTTCTGCCATGAACTGCTCGATCTCTTCCTCCTGGAGATCTCCTCGTCCGGCGCGCTCCACGGTGGC
CGCCAGGTCGTTGGAGATGCGACCCATGAGCTGCGAGAAGGCGTTGAGTCCGCCCTCGTTCCAGACCCGGCTGTAGACCACGCCCCCCTCGGCGTCGCGGGC
GCGCATGACCACCTGGGCCAGGTTGAGCTCCACGTGTCGCGTGAAGACGGCGTAGTTGCGCAGGCGCTGGAAAAGGTAGTTCAGGGTGGTGGCGGTGTGCTC
GGCGACGAAGAAGTACATGACCCAGCGCCGCAACGTGGATTCATTGATGTCCCCCAAGGCCTCCAGGCGCTCCATGGCCTCGTAGAAGTCCACGGCGAAGTT
GAAAAACTGGGAGTTGCGAGCGGACACGGTCAACTCCTCCTCCAGAAGACGGATGAGCTCGGCGACAGTGTCGCGCACCTCGCGCTCGAAGGCCACGGGGGG
CGCTTCTTCCTCTTCCACCTCTTCTTCCATGATTGCTTCTTCTTCTTCCTCAGCCGGGACGGGAGGGGGCGGCGGCGGGGGAGGGGCGCGGCGGCGGCGGCG
GCGCACCGGGAGGCGGTCGATGAAGCGCTCGATCATCTCCCCCCGCATGCGGCGCATGGTCTCGGTGACGGCGCGGCCGTTCTCCCGGGGGCGCAGCTCGAA
GACGCCGCCTCTCATTTCGCCGCGGGGCGGGCGGCCGTGAGGTAGCGAGACGGCGCTGACTATGCATCTTAACAATTGCTGTGTAGGTACGCCGCCAAGGGA
CCTGATTGAGTCCAGATCCACCGGATCCGAAAACCTTTGGAGGAAAGCGTCTATCCAGTCGCAGTCGCAAGGTAGGCTGAGCACCGTGGCGGGCGGGGGCGG
GTCGGGAGAGTTCCTGGCGGAGATGCTGCTGATGATGTAATTAAAGTAGGCGGTCTTGAGAAGGCGGATGGTGGACAGGAGCACCATGTCTTTGGGTCCGGC
CTGTTGGATGCGGAGGCGGTCGGCCATGCCCCAGGCCTCGTTCTGACACCGGCGCAGGTCTTTGTAGTAATCTTGCATGAGTCTTTCCACCGGCACTTCTTC
TCCTTCCTCTTCTTCATCTCGCCGGTGGTTTCTCGCGCCGCCCATGCGCGTGACCCCAAAGCCCCTGAGCGGCTGCAGCAGGGCCAGGTCGGCGACCACGCG
CTCGGCCAAGATGGCCTGCTGTACCTGAGTGAGGGTCCTCTCGAAGTCATCCATGTCCACGAAGCGGTGGTAGGCACCCGTGTTGATGGTGTAGGTGCAGTT
GGCCATGACGGACCAGTTGACGGTCTGGTGTCCCGGCTGCGAGAGCTCCGTGTACCGCAGGCGCGAGAAGGCGCGGGAATCGAACACGTAGTCGTTGCAAGT
CCGCACCAGATACTGGTAGCCCACCAGGAAGTGCGGCGGAGGTTGGCGATAGAGGGGCCAGCGCTGGGTGGCGGGGGCGCCGGGCGCCAGGTCTTCCAGCAT
GAGGCGGTGGTATCCGTAGATGTACCTGGACATCCAGGTGATGCCTGCGGCGGTGGTGGTGGCGCGCGCGTAGTCGCGGACCCGGTTCCAGATGTTTCGCAG
GGGCGAGAAGTGTTCCATGGTCGGCACGCTCTGGCCGGTGAGGCGCGCGCAGTCGTTGACGCTCTATACACACACAAAAACGAAAGCGTTTACAGGGCTTTC
GTTCTGTAGCCTGGAGGAAAGTAAATGGGTTGGGTTGCGGTGTGCCCCGGTTCGAGACCAAGCTGAGCTCAGCCGGCTGAAGCCGCAGCTAACGTGGTATTG
GCAGTCCCGTCTCGACCCAGGCCCTGTATCCTCCAGGATACGGTCGAGAGCCCTTTTGCTTTCTTGGCCAAGCGCCCGTGGCGCGATCTGGGATAGATGGTC
GCGATGAGAGGACAAAAGCGGCTCGCTTCCGTAGTCTGGAGAAACAATCGCCAGGGTTGCGTTGCGGCGTACCCCGGTTCGAGCCCCTATGGCGGCTTGGAT
CGGCCGGAACCGCGGCTAACGTGGGCTGTGGCAGCCCCGTCCTCAGGACCCCGCCAGCCGACTTCTCCAGTTACGGGAGCGAGCCCCTTTTGTTTTTTTATT
TTTTAGATGCATCCCGTGCTGCGGCAGATGCGCCCCTCGCCCCGGCCCGATCAGCAGCAGCAACAGCAGGCATGCAGACCCCCCTCTCCTCTCCCCGCCCCG
GTCACCACGGCCGCGGCGGCCGTGTCCGGTGCGGGGGGCGCGCTGGAGTCAGATGAGCCACCGCGGCGGCGACCTAGGCAGTATCTGGACTTGGAAGAGGGC
GAGGGACTGGCGCGGCTGGGGGCGAGCTCTCCAGAGCGCCACCCGCGGGTGCAGTTGAAAAGGGACGCGCGTGAGGCGTACCTGCCGCGGCAAAACCTGTTT
CGCGACCGCGGGGGCGAGGAGCCCGAGGAGATGCGGGACTGCAGGTTCCAAGCGGGGCGCGAGCTGCGCCGCGGCTTGGACAGACAGCGCCTGCTGCGCGAG
GAGGACTTTGAGCCCGACACGCAGACGGGCATCAGCCCCGCGCGCGCGCACGTGGCCGCGGCCGACCTGGTGACCGCCTACGAGCAGACGGTGAACCAGGAG
CGCAACTTCCAAAAAAGCTTCAACAACCACGTGCGCACGCTGGTGGCGCGCGAGGAGGTGACCCTGGGTCTCATGCATCTGTGGGACCTGGTGGAGGCGATC
GTGCAGAACCCCAGCAGCAAGCCCCTGACCGCGCAGCTGTTCCTGGTGGTGCAGCACAGCAGGGACAACGAGGCCTTCAGGGAGGCGCTGCTGAACATCACC
GAGCCGGAGGGGCGCTGGCTCCTGGACCTGATAAACATCCTGCAGAGCATAGTGGTGCAGGAGCGCAGCCTGAGCCTGGCCGAGAAGGTGGCGGCCATTAAC
TATTCTATGCTGAGCCTGGGCAAGTTCTACGCTCGCAAGATCTACAAGACCCCCTACGTGCCCATAGACAAGGAGGTGAAGATAGACAGCTTCTACATGCGC
ATGGCGCTGAAGGTGCTAACCCTGAGCGACGACCTGGGAGTGTACCGCAACGAGCGCATCCACAAGGCCGTGAGCGCCAGCCGGCGGCGCGAGCTGAGCGAC
CGCGAACTGATGCACAGTCTGCAGCGCGCGCTGACCGGCGCGGGCGAGGGCGACAGGGAGGTCGAGTCCTACTTTGACATGGGGGCCGACCTGCACTGGCAG
CCGAGCCGCCGCGCCCTGGAAGCGGCGGGGGCGTACGGCGGCCCCCTGGCGGCCGATGACGAGGAAGAGGAGGACTATGAGCTAGAGGAGGGCGAGTACCTG
GAGGACTGACCTGGCTGGTGGTGTTTTGGTATAGATGCAAGATCCGAACGTGGCGGACCCGGCGGTCCGGGCGGCGCTGCAGAGCCAGCCGTCCGGCATTAA
CTCCTCTGACGACTGGGCCGCGGCCATGGGTCGCATCATGGCCCTGACCGCGCGCAACCCCGAGGCCTTCAGGCAGCAGCCTCAGGCTAACCGGCTGGCGGC
CATCTTGGAAGCGGTAGTGCCCGCGCGCTCCAACCCCACCCACGAGAAGGTGCTGGCCATAGTCAACGCGCTGGCGGAGAGCAGGGCCATCCGGGCAGACGA
GGCCGGACTGGTGTACGATGCGCTGCTGCAGCGGGTGGCGCGGTACAACAGCGGCAACGTGCAGACCAACCTGGACCGCCTGGTGACGGACGTGCGCGAGGC
CGTGGCGCAGCGCGAGCGCTTGCATCAGGACGGCAACCTGGGCTCGCTGGTGGCGCTAAACGCCTTCCTTAGCACCCAGCCGGCCAACGTACCGCGGGGGCA
GGAGGACTACACCAACTTCTTGAGCGCGCTGCGGCTGATGGTGACCGAGGTCCCTCAGAGCGAGGTGTACCAGTCGGGGCCCGACTACTTCTTCCAGACCAG
CAGACAGGGCTTGCAAACCGTGAACCTGAGCCAGGCTTTCAAGAACCTGCGGGGGCTGTGGGGAGTGAAGGCGCCCACCGGCGACCGGGCTACGGTGTCCAG
CCTGCTAACCCCCAACTCGCGCCTGCTGCTGCTGCTGATCGCGCCCTTCACGGACAGCGGGAGCGTCTCGCGGGAGACCTATCTGGGCCACCTGCTGACGCT
GTACCGCGAGGCCATCGGGCAGGCGCAGGTGGACGAGCACACCTTCCAGGAGATCACCAGCGTGAGCCACGCGCTGGGGCAGGAGGACACGGGCAGCCTGCA
GGCGACCCTGAACTACCTGCTGACCAACAGGCGGCAGAAGATTCCCACGCTGCACAGCCTGACCCAGGAGGAGGAGCGCATCTTGCGCTACGTGCAGCAGAG
CGTGAGCCTGAACCTGATGCGCGACGGCGTGACGCCCAGCGTGGCGCTGGACATGACCGCGCGCAACATGGAACCGGGCATGTACGCTTCCCAGCGGCCGTT
CATCAACCGCCTGATGGACTACTTGCATCGGGCGGCGGCCGTGAACCCCGAGTACTTCACCAATGCCATTCTGAATCCCCACTGGATGCCCCCTCCGGGTTT
CTACAACGGGGACTTCGAGGTGCCTGAGGTCAACGATGGGTTCCTCTGGGATGACATGGATGACAGTGTGTTCTCCCCCAACCCGCTGCGCGCCGCGTCTCT
GCGATTGAAGGAGGGCTCTGACAGGGAAGGACCAAGGAGTCTGGCCTCCTCCCTGGCTCTGGGGGCGGTGGGCGCCACGGGCGCGGCGGCGCGGGGCAGCAG
CCCCTTCCCCAGCCTGGCGGACTCTCTGAATAGCGGGCGGGTGAGCAGGCCCCGCTTGCTAGGCGAGGAGGAGTATCTGAACAACTCCCTGCTGCAGCCCGT
GAGGGACAAAAACGCTCAGCGGCAGCAGTTTCCCAACAATGGGATAGAGAGCCTGGTGGACAAGATGTCCAGATGGAAGACGTATGCGCAGGAGTACAAGGA
GTGGGAGGACCGCCAGCCGCGGCCCCTGCCGCCCCCTAGACAGCGCTGGCAGCGGCGCGCGTCCAACCGCCGCTGGAGGCAGGGGCCCGAGGACGATGATGA
CTCTGCAGATGACAGCAGCGTGTTGGACCTGGGCGGGAGCGGGAACCCCTTTTCGCACCTGCGCCCACGCCTGGGCAAGATGTTTTAAAAGAGAAAAATAAA
AACTCACCAAGGCCATGGCGACGAGCGTTGGTTTTTTGTTCCCTTCCTTAGTATGCGGCGCGCGGCGATGTTCGAGGAGGGGCCTCCCCCCTCTTACGAGAG
CGCGATGGGAATTTCTCCTGCGGCGCCCCTGCAGCCTCCCTACGTGCCTCCTCGGTACCTGCAACCTACAGGGGGGAGAAATAGCATCTGTTACTCTGAGCT
GCAGCCCCTGTACGATACCACCAGACTGTACCTGGTGGACAACAAGTCCGCGGACGTGGCCTCCCTGAACTACCAGAACGACCACAGCGATTTTTTGACCAC
GGTGATCCAAAACAACGACTTCACCCCAACCGAGGCCAGTACCCAGACCATAAACCTGGACAACAGGTCGAACTGGGGCGGCGACCTGAAGACTATCCTGCA
CACCAATATGCCCAACGTGAACGAGTTCATGTTCACCAACTCTTTTAAGGCGCGGGTGATGGTGGCGCGCGAGCAGGGGGAGGCGAAGTACGAGTGGGTGGA
CTTCACGCTGCCCGAGGGCAACTACTCAGAGACCATGACTCTCGACCTGATGAACAATGCGATCGTGGAACACTATCTGAAAGTGGGCAGGCAGAACGGGGT
GAAGGAGAGCGATATCGGGGTCAAGTTTGACACCAGAAACTTCCGTCTGGGCTGGGACCCTGTGACCGGGCTGGTCATGCCGGGGGTCTACACCAACGAGGC
CTTTCATCCCGATATAGTGCTCCTGCCCGGCTGTGGGGTGGACTTCACCCAGAGCCGGCTGAGCAACCTGCTGGGCGTTCGCAAGCGGCAACCTTTCCAGGA
GGGTTTCAAGATCACCTATGAGGATCTGGAGGGGGGCAACATTCCCGCGCTCCTTGATCTGGACGCCTACGAGGAGAGCTTGAAACCCGAGGAGAGCGCTGG
CGACAGCGGCGAGAGTGGCGAGGAGCAAGCCGGCGGCGGCGGCAGCGCGTCGGTAGAAAACGAAAGTACTCCCGCAGTGGCGGCGGACGCTGCGGAGGTCGA
GCCGGAGGCCATGCAGCAGGACGCAGAGGAGGGCGCGCAGGAGGACATGAACAATGGGGAGATCAGGGGCGACACTTTCGCCACCCGGGGCGAAGAAAAAGA
GGCAGAGGCGGCGGCGGCGACGGCGGAAGCCGAAACCGAGGCAGAGGCAGAGCCCGAGACCGAAGTTATGGAAGACATGAATGATGGAGAACGTAGGGGTGA
CACGTTTGCCACCCGGGGCGAAGAGAAGGCGGCGGAGGCAGAAGCCGCGGCTGAGGAGGCGGCTGCGGCTGCGGCCAAGGCTGAGGCTGCGGCTGAGGCTAA
GGTCGAAGCCGATGTTGCGGTTGAGGCTCAGGCTGAGGAGGAGGCGGCGGCTGAAGCAGTTAAGGAAAAGGCCCAGGCAGAGCAGGAAGAGAAAAAACCTGT
CATTCAACCTCTAAAAGAAGATAGCAAAAAGCGCAGTTACAACGTCATTGAGGGCAGCACCTTTACCCAATACCGCAGCTGGTACCTGGCTTACAACTACGG
CGACCCGGTCAAGGGGGTGCGCTCGTGGACCCTGCTCTGCACGCCGGACGTCACCTGCGGCTCCGAGCAGATGTACTGGTCGCTGCCAAACATGATGCAAGA
CCCGGTGACCTTCCGTTCCACGCGGCAGGTTAGCAACTTTCCGGTGGTGGGCGCCGAACTGCTGCCAGTACACTCCAAGAGTTTTTACAACGAGCAGGCCGT
CTACTCCCAGCTGATCCGCCAGGCCACCTCTCTGACCCACGTGTTCAATCGCTTTCCCGAGAACCAGATTTTGGCGCGCCCGCCGGCCCCCACCATCACCAC
CGTCAGTGAAAACGTTCCTGCCCTCACAGATCACGGGACGCTACCGCTGCGCAACAGCATCTCAGGAGTCCAGCGAGTGACCATTACTGACGCCAGACGCCG
GACCTGCCCCTACGTTTACAAGGCCTTGGGCATAGTCTCGCCGCGCGTCCTCTCCAGTCGCACTTTTTAAAACACATCCACCCACACGCTCCAAAATCATGT
CCGTACTCATCTCGCCCAGCAACAACACCGGCTGGGGGCTGCGCGCACCCAGCAAGATGTTTGGAGGGGCAAGGAAGCGCTCCGACCAGCACCCCGTGCGCG
TGCGCGGCCACTACCGCGCGCCCTGGGGTGCGCACAAGCGCGGGCGCACAGGGCGCACCACTGTGGATGATGTCATTGACTCCGTAGTGGAGCAGGCGCGCC
ACTACACACCCGGCGCGCCGACCGCCTCCGCCGTGTCCACCGTGGACCAGGCGATCGAAAGCGTGGTACAGGGGGCGCGGCACTATGCCAACCTTAAAAGTC
GCCGCCGCCGCGTGGCGCGCCGCCATCGCCGGAGACCCCGGGCTACTGCCGCCGCGCGCCTTACCAAGGCTCTGCTCAAGCGCGCCAGGCGAACTGGCCACC
GGGCCGCCATGAGGGCCGCACGGCGGGCTGCCGCTGCCGCGAGCGCCGTGGCCCCGCGGGCACGAAGGCGCGCGGCCGCTGCCGCCGCCGCCGCCATTTCCA
GCTTGGCCTCGACGCGGCGCGGTAACATATACTGGGTGCGCGACTCGGTGAGCGGCACACGTGTGCCCGTGCGCTTTCGCCCCCCACGGAATTAGCACAAGA
CAACATACACACTGAGTCTCCTGCTGTTGTGTATCCCAGCGGCGACCGTCAGCAGCGGCGACATGTCCAAGCGCAAAATTAAAGAAGAGATGCTCCAGGTCA
TCGCGCCGGAGATCTATGGGCCCCCGAAGAAGGAGGAGGAGGATTACAAGCCCCGCAAGCTAAAGCGGGTCAAAAAGAAAAAGAAAGATGATGACGTTGACG
AGGCGGTGGAGTTTGTCCGCCGCATGGCGCCCAGGCGCCCTGTGCAGTGGAAGGGTCGGCGCGTGCAGCGAGTCCTGCGCCCCGGCACCGCGGTGGTCTTTA
CGCCCGGCGAGCGTTCCACGCGCACTTTCAAGCGGGTGTACGATGAGGTGTACGGCGACGAGGATCTGTTGGAGCAGGCCAACCATCGATTTGGGGAGTTTG
CATATGGGAAACGGCCTCGCGAGAGTCTAAAAGAGGACCTGCTGGCGCTACCGCTGGACGAGGGCAATCCCACCCCGAGTCTGAAGCCGGTGACCCTGCAAC
AGGTGCTGCCTTTGAGCGCGCCCAGCGAGCAGAAGCGAGGGTTAAAGCGCGAGGGCGGGGACCTGGCACCCACCGTGCAGTTGATGGTGCCCAAGCGGCAGA
AGCTGGAGGACGTGCTGGAGAAAATGAAAGTAGAGCCCGGGATCCAGCCCGAGATCAAGGTCCGCCCTATCAAGCAGGTGGCGCCCGGCGTGGGAGTCCAGA
CCGTGGACGTTAGGATTCCCACGGAGGAGATGGAAACCCAAACCGCCACTCCCTCTTCGGCAGCAAGCGCCACCACCGGCGCCGCTTCGGTAGAGGTGCAGA
CGGACCCCTGGCTACCCGCCGCCACTATCGCCGTCGCCGCCGCCCCCCGTTCGCGCGGACGCAAGAGAAATTATCCAGCGGCCAGCGCGCTTATGCCCCAGT
ATGCGCTGCATCCATCCATCGCGCCCACCCCCGGCTACCGCGGGTACTCGTACCGCCCGCGCAGATCAGCCGGCACTCGCGGCCGCCGCCGCCGTGCGACCA
CAACCAGCCGCCGCCGTCGCCGCCGCCGCCAGCCAGTGCTGACCCCCGTGTCTGTAAGGAAGGTGGCTCGCTCGGGGAGCACGCTGGTGGTGCCCAGAGCGC
GCTACCACCCCAGCATCGTTTAAAGCCGGTCTCTGTATGGTTCTTGCAGATATGGCCCTCACTTGTCGCCTTCGCTTCCCGGTGCCGGGATACCGAGGAAGA
ACTCACCGCCGCAGGGGCATGGCGGGCAGCGGTCTCCGCGGCGGCCGTCGCCATCGCCGGCGCGCAAAGAGCAGGCGCATGCGCGGCGGTGTGTTGCCCCTG
CTGGTCCCGCTACTCGCCGCGGCGATCGGCGCCGTGCCCGGGATCGCCTCCGTGGCCCTGCAGGCGTCCCAGAAACATTGACTCTTGCAACCTTGCAAGCTT
GCATTTTTGGAGGAAAAAATAAAAAAGTCTAGACTCTCACGCTCGCTTGGTCCTGTGACTATTTTGTAGAAAAAAGATGGAAGACATCAACTTTGCGTCGCT
GGCCCCGCGTCACGGCTCGCGCCCGTTCATGGGAGACTGGACAGATATCGGCACCAGCAATATGAGCGGTGGCGCCTTCAGCTGGGGCAGTCTGTGGAGCGG
CCTTAAAAATTTTGGTTCCACCATTAAGAACTATGGCAACAAAGCGTGGAACAGCAGCACGGGTCAGATGCTGAGAGACAAGTTGAAAGAGCAGAACTTCCA
GGAGAAGGTGGCGCAGGGCCTGGCCTCTGGCATCAGCGGGGTGGTGGACATAGCTAACCAGGCCGTGCAGAAAAAGATAAACAGTCATCTGGACCCCCGCCC
TCAGGTGGAGGAAACGCCTCCAGCCATGGAGACGGTGTCTCCCGAGGGCAAAGGCGAAAAGCGCCCGCGGCCCGACAGGGAAGAGACCCTGGTGTCACACAC
CGAGGAGCCGCCCTCTTACGAGGAGGCAGTCAAGGCCGGCCTGCCCACCACTCGCCCCATAGCTCCCATGGCCACCGGTGTGGTGGGTCACAGGCAACACAC
CCCCGCAACACTAGATCTGCCCCCGCCGTCCGAGCCGACTCGCCAGCCAAAGGCGGTGACGGTGTCCGCTCCCTCCACTTCCGCCGCCAACAGAGTGCCTCT
GCGCCGCGCTGCGAGCGGCCCCCGGGCCTCGCGAGTCAGCGGCAACTGGCAGAGCACACTGAACAGCATCGTGGGCCTGGGAGTGAGGAGTGTGAAGCGCCG
CCGTTGCTACTGAATGAGCAAGCTAGCTAACGTGTTGTATGTGTGTATGCGTCCTATGTCGCCGCCAGAGGAGCTGTTGAGCCGCCGGCGCCGTCTGCACTC
CAGCGAATTTCAAGATGGCGACCCCATCGATGATGCCTCAGTGGTCGTACATGCACATCTCGGGCCAGGACGCTTCGGAGTACCTGAGCCCCGGGCTGGTGC
AGTTCGCCCGCGCCACAGACACCTACTTCAACATGAGTAACAAGTTCAGGAACCCCACTGTGGCGCCCACCCACGATGTGACCACGGACCGGTCGCAGCGCC
TGACGCTGCGGTTCATCCCCGTGGATCGGGAGGACACCGCTTACTCTTACAAGGCGCGGTTCACGCTGGCCGTGGGCGACAACCGCGTGCTGGACATGGCCT
CCACTTACTTTGACATCCGGGGGGTGCTGGACAGGGGCCCCACTTTTAAGCCCTACTCGGGCACTGCCTACAACCCCCTGGCCCCCAAGGGCGCCCCCAATT
CTTGTGAGTGGGAACAAGAGGAAAATCAGGTGGTCGCTGCAGATGATGAACTTGAAGATGAAGAAGCGCAAGCACAAGAGGAAGCCCCTGTGAAAAAAATTC
ATGTATATGCTCAGGCGCCTCTTTCTGGCGAAAAGATTTCCAAGGATGGTATCCAAATAGGTACTGAAGTCGTAGGAGATACATCTAAGGACACTTTTGCAG
ATAAAACATTCCAACCCGAACCTCAGATAGGCGAGTCTCAGTGGAACGAGGCTGATGCCACAGCAGCAGGAGGTAGAGTTTTGAAAAAGACTACCCCTATGA
GACCTTGCTATGGATCCTATGCCAGGCCTACCAATGCCAACGGGGGTCAAGGAATTATGGTTGCCAATGAACAAGGAGTGTTGGAGTCTAAAGTAGAAATGC
AATTTTTCTCTAACACCACAACCCTTAATGCGCGGGATGGAACCGGCAATCCCGAACCAAAGGTGGTGTTGTACAGCGAAGATGTCCACTTGGAATCTCCCG
ATACTCATCTGTCTTACAAGCCCAAAAAGGATGATGTTAATGCCAAAATCATGTTGGGTCAGCAAGCCATGCCCAACAGACCCAACCTCATTGGATTTAGAG
ATAATTTCATTGGGCTTATGTTTTACAACAGCACCGGTAACATGGGAGTGCTGGCGGGTCAGGCCTCTCAGTTGAATGCTGTGGTGGACTTGCAGGATAGAA
ACACAGAACTGTCATATCAGCTTCTGCTTGATTCAATTGGGGATAGAACCAGATACTTCTCCATGTGGAACCAGGCAGTGGATAGCTATGATCCAGATGTCA
GAATTATTGAAAACCATGGGACTGAGGATGAACTGCCCAACTACTGCTTCCCTTTGGGCGGCATAGGAGTTACTGATACTTATCAAGGGATAAAAAATACCA
ATGGCAATGGTCAGTGGACCAAAGATGATCAGTTCGCGGACCGCAACGAAATAGGGGTGGGAAACAACTTCGCCATGGAGATCAACATCCAGGCCAACCTTT
GGAGAAACTTCCTCTATGCAAACGTGGGGCTCTACCTGCCAGACAAGCTCAAGTACAACCCCACCAACGTGGACATCTCTGACAACCCCAACACCTATGACT
ACATGAACAAGCGGGTGGTGGCCCCTGGCCTGGTGGACTGCTTTGTCAATGTGGGAGCCAGGTGGTCCCTGGACTACATGGACAACGTCAACCCCTTCAACC
ACCACCGCAATGCGGGTCTGCGCTACCGCTCCATGATCCTGGGCAACGGGCGCTATGTGCCCTTTCACATCCAGGTACCCCAGAAGTTCTTTGCCATCAAGA
ACCTCCTGCTCCTGCCCGGCTCCTACACCTACGAGTGGAACTTCAGGAAGGATGTGAACATGGTCCTACAGAGCTCTCTGGGCAATGACCTTAGGGTGGATG
GGGCCAGCATCAAGTTTGACAGCATCACCCTCTATGCTACATTTTTCCCCATGGCCCACAACACCGCCTCCACGCTTGAGGCCATGCTGAGAAACGACACCA
ACGACCAGTCCTTTAATGACTACCTCTCTGGGGCCAACATGCTCTACCCAATCCCAGCCAAGGCCACCAACGTGCCCATCTCCATCCCCTCTCGCAACTGGG
CCGCCTTTAGAGGCTGGGCCTTTACCCGCCTTAAGACCAAGGAGACCCCCTCCCTGGGCTCGGGTTTTGATCCCTACTTTGTTTACTCGGGATCCATCCCCT
ACCTGGATGGCACCTTCTACCTCAACCACACTTTCAAGAAGATATCCATCATGTATGACTCCTCCGTCAGCTGGCCGGGCAACGACCGCTTGCTCACCCCCA
ATGAGTTCGAGGTCAAGCGCGCCGTGGACGGCGAGGGCTACAACGTGGCCCAGTGCAACATGACCAAGGACTGGTTCCTGGTGCAGATGCTGGCCAACTACA
ACATAGGCTACCAGGGCTTTTACATCCCAGAGAGCTACAAGGACAGGATGTACTCCTTCTTCAGAAATTTCCAACCCATGAGCCGACAGGTGGTGGACGAGA
CCAATTACAAGGACTATCAAGCCATTGGCATCACCCACCAGCACAACAACTCGGGTTTCGTGGGCTACCTGGCGCCCACCATGCGCGAGGGTCAGGCCTACC
CCGCCAACTTCCCCTACCCCTTGATAGGCAAGACCGCGGTCGACAGCGTCACCCAGAAAAAGTTCCTCTGCGACCGCACCCTCTGGCGCATCCCCTTCTCTA
GCAACTTCATGTCCATGGGTGCGCTCACGGACCTGGGCCAAAACCTGCTTTATGCCAACTCTGCCCATGCGCTGGACATGACTTTTGAGGTGGACCCCATGG
ACGAGCCCACCCTTCTCTATATTGTGTTTGAAGTGTTCGACGTGGTCAGAGTGCACCAGCCGCACCGCGGTGTCATCGAGACCGTGTACCTGCGTACGCCCT
TCTCAGCCGGCAACGCCACCACCTAAGGAGACAGCGCCGCCGCCGCCTGCATGACGGGTTCCACCGAGCAAGAGCTCAGGGCCATTGCCAGAGACCTGGGAT
GCGGACCCTATTTTTTGGGCACCTATGACAAACGCTTCCCGGGCTTTATCTCCCGAGACAAGCTCGCCTGCGCCATTGTCAACACGGCCGCGCGCGAGACCG
GGGGCGTGCACTGGCTGGCCTTTGGCTGGGACCCGCGCTCCAAAACTTGCTACCTCTTTGACCCCTTTGGCTTCTCCGATCAGCGCCTCAGGCAGATTTATG
AGTTTGAGTACGAGGGGCTGCTGCGCCGCAGCGCGCTCGCCTCCTCGCCCGACCGCTGCATCACCCTTGAGAAGTCCACCGAAACCGTGCAGGGGCCCCACT
CGGCCGCCTGCGGTCTCTTCTGTTGCATGTTTTTGCACGCCTTTGTGCACTGGCCTCAGAGTCCCATGGATTGCAACCCCACCATGAACTTGCTAAAGGGAG
TGCCCAACGCCATGCTCCAGAGCCCCCAGGTCCAGCCCACCCTGCGCCGCAACCAGGAACAGCTTTACCGCTTCCTGGAGCGCCACTCCCCCTACTTCCGCA
GCCACAGCGCGCGCATCCGGGGGGCCACCTCTTTTTGCCACTTGCAAGAAAACATGCAAGACGGAAAATGATGTACAGCATGCTTTTAATAAATGTAAAGAC
TGTGCACTTTAATTATACACGGGCTCTTTCTGGTTATTTATTCAACACCGCCGTCGCCATTTAGAAATCGAAAGGGTTCTGCCGTGCGTCGCCGTGCGCCAC
GGGCAGAGACACGTTGCGATACTGGAAGCGGCTCGCCCACTTGAACTCGGGCACCACCATGCGGGGCAGTGGTTCCTCGGGGAAGTTCTCGCTCCACAGGGT
GCGGGTCAGCTGCAGCGCGCTCAGGAGGTCGGGAGCCGAGATCTTGAAGTCGCAGTTGGGGCCGGAACCCTGCGCGCGCGAGTTGCGGTACACGGGGTTGCA
GCACTGGAACACCAGCAGGGCCGGATTATTCACGCTGGCCAGCAGGCTCTCGTCGCTGATCATGTCGCTGTCCAGATCCTCCGCGTTGCTCAGGGCGAATGG
GGTCATCTTGCAGACCTGCCTGCCCAGGAAAGGCGGGAGCCCAGGCTTGCCGTTGCAGTCGCAGCGCAGGGGCATTAGCAGGTGCCCACGGCCCGACTGCGC
CTGCGGGTACAACGCGCGCATGAAGGCTTCGATCTGCCTAAAAGCCACCTGGGTCTTGGCTCCCTCCGAAAAGAACATCCCACAGGACTTGCTGGAGAACTG
GTTCGCGGGACAGCTGGCATCGTGCAGGCAGCAGCGCGCGTCAGTGTTGGCAATCTGCACCACGTTGCGACCCCACCGGTTTTTCACTATCTTGGCCTTGGA
AGCCTGCTCCTTTAGCGCGCGCTGGCCGTTCTCGCTGGTCACATCCATCTCTATCACCTGTTCCTTGTTGATCATGTTTGTCCCGTGCAGACACTTTAGGTC
GCCCTCCGTCTGGGTGCAGCGGTGCTCCCACAGCGCGCAACCGGTGGGCTCCCAATTCTTGTGGGTCACCCCCGCGTAGGCCTGCAGGTAGGCCTGCAGGAA
GCGCCCCATCATGGTCATAAAGGTCTTCTGGCTCGTAAAGGTCAGCTGCAGGCCGCGATGCTCTTCGTTCAGCCAGGTCTTGCAGATGGCGGCCAGCGCCTC
GGTCTGCTCGGGCAGCATCTTAAAATTTGTCTTCAGGTCGTTATCCACGTGGTACTTGTCCATCATGGCACGCGCCGCCTCCATGCCCTTCTCCCAGGCGGA
CACCATGGGCAGGCTTAGGGGGTTTATCACTTCCAGCGGCGAGGACACCGTACTTTCGATTTCTTCTTCCTCCCCCTCTTCCCGGCGCGCGCCCCCGCTGTT
GCGCGCTCTTACCGCCTGCACCAAGGGGTCGTCTTCAGGCAAGCGCCGCACCGAGCGCTTGCCGCCCTTGACCTGCTTGATCAGTACCGGCGGGTTGCTGAA
GCCCACCATGGTCAGCGCCGCCTGCTCTTCTTCGTCTTCGCTGTCTACCACTATTTCTGGGGAGGGGCTTCTCCGCTCTGCGGCAAAGGCGGCGGATCGCTT
CTTTTTTTTCTTGGGAGCCGCCGCGATGGAGTCCGCCACGGCGACCGAGGTCGAGGGCGTGGGGCTGGGGGTGCGCGGTACCAGGGCCTCGTCGCCCTCGGA
CTCTTCCTCTGACTCCAGGCGGCGGCGGAGTCGCTTCTTTGGGGGCGCGCGCGTCAGCGGCGGCGGAGACGGGGACGGGGACGGGGACGGGACGCCCTCCAC
AGGGGGTGGTCTTCGCGCAGACCCGCGGCCGCGCTCGGGGGTCTTCTCGCGCTGGTCTTGGTCCCGACTGGCCATTGTATCCTCCTCCTCCTAGGCAGAGAG
ACATAAGGAGTCTATCATGCAAGTCGAGAAGGAGGAGAGCTTAACCACCCCCTCAGAGACCGCCGATGCGCCCGCCGTCGCCGTCGCCCCCGCTACCGCCGA
CGCGCCCGCCACACCGAGCGACACCCCCACGGACCCCCCCGCCGACGCACCCCTGTTCGAGGAAGCGGCCGTGGAGCAGGACCCGGGCTTTGTCTCGGCAGA
GGAGGATTTGCAAGAGGAGGAGAATAAGGAGGAGAAGCCCTCAGTGCCAAAAGATCATAAAGAGCAAGACGAGCACGACGCAGACGCACACCAGGGTGAAGT
CGGGCGGGGGGACGGAGGGCATGGCGGCGCCGACTACCTAGACGAAGGAAACGACGTGCTCTTGAAGCACCTGCATCGTCAGTGCGCCATCGTCTGCGACGC
TCTGCAGGAGCGCAGCGAGGTGCCCCTCAGCGTGGCGGAGGTCAGCCGCGCCTACGAGCTCAGCCTCTTTTCCCCCCGGGTGCCCCCCCGCCGCCGCGAAAA
CGGCACATGCGAGCCCAACCCGCGCCTCAACTTCTACCCCGCCTTTGTGGTGCCCGAGGTCCTGGCCACCTATCACATCTTCTTTCAAAATTGCAAGATCCC
CATCTCGTGCCGCGCCAACCGTAGCCGCGCCGATAAGATGCTGGCCCTGCGCCAGGGCGACCACATACCTGATATCGCCGCTTTGGAAGATGTGCCAAAGAT
CTTCGAGGGTCTGGGGCGCAACGAGAAGCGGGCAGCAAACTCTCTGCAACAGGAAAACAGCGAAAATGAGAGTCACACTGGAGCGCTGGTGGAGCTGGAGGG
CGACAACGCCCGCCTGGCGGTGCTCAAGCGCAGCATCGAGGTCACCCACTTTGCCTACCCCGCGCTCAACCTGCCCCCCAAAGTCATGAACGCGGTCATGGA
CGGGCTGATCATGCGCCGCGGCCGGCCCCTCGCTCCAGATGCAAACTTGCATGAGGAGACCGAGGACGGTCAGCCCGTGGTCAGCGACGAGCAGCTGACGCG
CTGGCTGGAGAGCGCGGACCCCGCCGAACTGGAGGAGCGGCGCAAGATGATGATGGCCGCGGTGCTGGTCACCGTAGAGCTGGAGTGTCTGCAGCGCTTCTT
CGGTGACCCCGAGATGCAGAGAAAGGTCGAGGAGACCCTACACTACACCTTCCGCCAGGGCTACGTGCGCCAGGCTTGCAAGATCTCCAACGTGGAGCTCAG
CAACCTGGTGTCCTACCTGGGCATCTTGCATGAAAACCGCCTTGGGCAGAGCGTGCTACACTCCACCCTGCGCGGGGAGGCGCGCCGCGACTACGTGCGCGA
CTGCGTTTACCTCTTCCTCTGCTACACCTGGCAGACGGCCATGGGGGTCTGGCAGCAGTGCCTGGAGGAGCGCAACCTCAAGGAGCTGGAGAAGCTTCTGCA
GCGCGCGCTCAAAGACCTCTGGACGGGCTTCAACGAGCGCTCGGTGGCCGCCGCGCTAGCCGACCTCATCTTCCCCGAGCGCCTGCTCAAAACCCTCCAGCA
GGGGCTGCCCGACTTCACCAGCCAAAGCATGTTGCAAAATTTTAGGAACTTTATCCTGGAGCGTTCTGGCATCCTACCCGCCACCTGCTGCGCCCTGCCCAG
CGACTTTGTCCCCCTCGTGTACCGCGAGTGCCCCCCGCCGCTGTGGGGCCACTGCTACCTGTTCCAACTGGCCAACTACCTGTCCTACCACGCGGACCTCAT
GGAGGACTCCAGCGGCGAGGGGCTCATGGAGTGCCACTGCCGCTGCAACCTCTGCACGCCCCACCGCTCCCTGGTCTGCAACACCCAACTGCTCAGCGAGAG
TCAGATTATCGGTACCTTCGAGCTACAGGGTCCGTCCTCCTCAGACGAGAAGTCCGCGGCTCCGGGGCTAAAACTCACTCCGGGGCTGTGGACTTCCGCCTA
CCTGCGCAAATTTGTACCTGAAGACTACCACGCCCACGAAATCAGGTTTTACGAGGACCAATCCCGCCCGCCCAAGGCGGAGCTGACCGCCTGCGTCATCAC
CCAGGGCGAGATCCTAGGCCAATTGCAAGCCATCCAAAAAGCCCGCCAAGAGTTTTTGCTGAAGAGGGGTCGGGGGGTGTATCTGGACCCCCAGTCGGGTGA
GGAGCTCAACCCGGTTCCCCCGCTGCCACCGCCGCGGGACCTTGCTTCCCAGGATAAGCATCGCCATGGCTCCCAGAAAGAAGCAGCAGCGGCCGCCGCTGC
CGCCGCCCCACATGCTGGAGGAAGAGGAGGAATACTGGGACAGTCAGGCAGAGGAGGTTTCGGACGAGGAGGAGCCGGAGACGGAGATGGAAGAGTGGGAGG
AGGACAGCTTAGACGAGGAGGCTTCCGAAGCCGAAGAGGCAGGCGCAACACCGTCACCCTCGGCCGCAGCCCCCTCGCAGGCGCCCCCGAAGTCCGCTCCCA
GCATCAGCAGCAACAGCAGCGCTATAACCTCCGCTCCTCCACCGCCGCGACCCACGGCCGACCGCAGACCCAACCGTAGATGGGACACCACCGGAACCGGGG
CCGGTAAGTCCTCCGGGAGAGGCAAGCAAGCGCAGCGCCAAGGCTACCGCTCGTGGCGCGCTCACAAGAACGCCATAGTCGCTTGCTTGCAAGACTGCGGGG
GGAACATCTCCTTCGCCCGCCGCTTCCTGCTCTTCCACCACGGTGTGGCCTTCCCCCGTAACGTCCTGCATTACTACCGTCATCTCTACAGCCCCTACTGCG
GCGGCAGTGAGCCAGAGGCGGCCAGCGGCGGCGGCGCCCGTTTCGGTGCCTAGGAAGACCCAGGGCAAGACTTCAGCCAAGAAACTCGCGGCGACCGCGGCG
AACGCGGTCGCGGGGGCCCTGCGCCTGACGGTGAACGAACCCCTGTCGACCCGCGAACTGAGGAACCGAATCTTCCCCACTCTCTATGCCATCTTCCAGCAG
AGCAGAGGGCAGGATCAGGAACTGAAAGTAAAAAACAGGTCTCTGCGCTCCCTCACCCGCAGCTGTCTGTATCACAAGAGCGAAGACCAGCTTCGGCGCACG
CTGGAGGACGCTGAGGCACTCTTCAGCAAATACTGCGCGCTCACTCTTAAGGACTAGCTCCGCGCCCTTCTCGAATTTAGGCGGGAACGCCTACGTCATCGC
AGCGCCGCCGTCATGAGCAAGGACATTCCCACGCCATACATGTGGAGCTATCAGCCGCAGATGGGACTCGCGGCGGGCGCCTCCCAAGACTACTCCACCCGC
ATGAACTGGCTCAGTGCCGGCCCACACATGATCTCACAGGTTAATGACATCCGCACCCATCGAAACCAAATATTGGTGAAGCAGGCGGCAATTACCACCACG
CCCCGCAATAATCCCAACCCCAGGGAGTGGCCCGCGTCCCTGGTGTATCAGGAAATTCCCGGCCCCACCACCGTACTACTTCCGCGTGATTCCCAGGCCGAA
GTCCAAATGACTAACTCAGGGGCACAGCTCGCGGGCGGCTGTCGTCACAGGGTGCGGCCTCCTCGCCAGGGTATAACTCACCTGGAGATCCGAGGCAGAGGT
ATTCAGCTCAACGACGAGTCGGTGAGCTCCTCGCTCGGTCTCAGACCTGACGGGACCTTCCAGATAGCCGGAGCCGGCCGATCTTCCTTCACGCCCCGCCAG
GCGTACCTGACTCTGCAGAGCTCGTCCTCGGCGCCGCGCTCGGGCGGCATCGGGACTCTCCAGTTCGTGCAGGAGTTTGTGCCCTCGGTCTACTTCAACCCC
TTCTCGGGCTCTCCCGGTCGCTACCCGGACCAGTTTATCCCGAACTTTGACGCCGCGAGGGACTCGGTGGACGGCTACGACTGAATGTCGGGTGGACCCGGT
GCAGAGCAACTTCGCCTGAAGCACCTTGACCACTGCCGCCGCCCTCAGTGCTTTGCCCGCTGTCAGACCGGTGAGTTCCAGTACTTTTCCCTGCCCGACTCG
CACCCGGACGGCCCGGCGCACGGGGTGCGCTTTTTCATCCCGAGTCAGGTCCGCTCTACCCTAATCAGGGAGTTCACCGCCCGTCCCCTACTGGCGGAGTTG
GAAAAGGGGCCTTCTATCCTAACCATTGCCTGCATTTGCTCTAACCCTGGATTACACCAAGATCTTTGCTGTCATTTGTGTGCTGAGTATAATAAAGGCTGA
GATCAGAATCTACTCGGGCTCCTGTCGCCATCCTGTCAACGCCACCGTCCAAGCCCGGCCCGATCAGCCCGAGGTGAACCTCACCTGTGGTCTGCACCGGCG
CCTGAGGAAATACCTAGCTTGGTACTACAACAGCACTCCCTTTGTGGTTTACAACAGCTTTGACCAGGACGGGGTCTCACTGAGGGATAACCTCTCGAACCT
GAGCTACTCCATCAGGAAGAACAACACCCTCGAGCTACTTCCTCCTTACCTGCCCGGGACTTACCAGTGTGTCACCGGCCCCTGCACCCACACCCACCTGTT
GATCGTAAACGACTCTCTTCCGAGAACAGACCTCAATAACTCCTCTCCGCAGTTCCCCAGAACAGGAGGTGAGCTCAGGAAACCCCGGGTAAAGAAGGGTGG
ACAAGAGTTAACACTTGTGGGGTTTCTGGTATATGTGACGCTGGTGGTGGCTCTTTTGATTAAGGCTTTTCCTTCCATGTCTGAACTATCCCTCTTCTTTTA
TGAACAACTCGACTAGTGCTAACGGGACCCTACCCAACGAATCGGGATTGAATATCGGTAACCAGGTTGCAGTTTCACTTTTGATTACCTTCATAGTCCTCT
TCCTGCTAGTGCTGTCGCTTCTGTGCCTGCGGATCGGGGGCTGCTGCATCCACGTTTATATCTGGTGCTGGCTGTTTAGAAGGTTCGGAGACCACCGCAGGT
AGAATAATGCTGCTTACCCTCTTTGTCCTGGCGCTGGCTGCCAGCTGCCAAGCCTTTTCCGAGGCTGACTTCATAGAGCCCCAGTGCAATATCACTTATAAA
TCTGAACGTGCCATCTGTACTATTCTAATCAAATGTGTTACTCAACACGATAAGGTGACTGTTAAATACAAAGATCAATTAAAAAAAGACGCACTTTACAGC
AGCTGGCAACCAGGAGATGATCAAAAATACAATGTAACCGTCTTCCAGGGCAAACTCTCCAAAACTTACAATTACAATTTCCCATTTGAGCAGATGTGTGAC
TTTGTCATGTACATGGAAAAGCAGTACAAGCTGTGGCCTCCAACTCCCCAGGGCTGTGTGGAAAATCCAGGCTCTTTCTGTATGATCTCTCTCTGTGTAACT
GTGCTGGCACTAATACTCACGCTTCTGTATCTCAGATTTAAATCAAGGCAAAGCTTCATTGATGAAAAGAAAATGCCATAATCGCTCAACGCTTGATTGCTA
ACACCGGGTTTTTATCCGCAGAATGATTGGAATCACCCTACTAATCACCTCCCTCCTTGCGATTGCCCATGGGTTGGAACGAATCGAAGTCCCTGTGGGGGC
CAATGTTACCCTGGTGGGGCCTGTCGGCAATGCTACATTAATGTGGGAAAAATATACTAAAAATCAATGGGTTTCTTACTGCACTAACAAAAACAGCCACAA
GCCCAGAGCCATCTGCGATGGGCAAAATCTAACCTTGATTGATGTTCAATTGCTGGATGCGGGCTACTATTATGGGCAGCTGGGTACAATGATTAATTACTG
GAGACCCCACAGAGATTACATGCTTCACGTAGTAAAGGGTCCCATTAGCAGCCCAACCACCACCTCTACCACACCCACTACCACCACTACTCCCACCACCAG
CACTGCCGCCCAGCCTCCTCATAGCAGAACAACCACTTTTATCAATTCCAAGTCCCACTCCCCCCACATTGCCGGCGGGCCCTCCGCCTCAGACTCCGAGAC
CACCGAGATCTGCTTCTGCAAATGCTCTGACGCCATTGCCCAGGATTTGGAAGATCACGAGGAAGATGAGCATGACTACGCAGATGCATGCCAGGCATCAGA
GGCAGAAGCGCTACCGGTGGCCCTAAAACAGTATGCAGACTCCCACACCACCCCCAACCTTCCTCCACCTTCCCAGAAGCCAAGTTTCCTGGGGGAAAATGA
AACTCTGCCTCTTTCCATACTAGCTCTGACATCTGTTGCTATTTTGGCCGCTCTGCTGGTGCTTCTATGCTCTATATGCTACCTGATCTGCTGCAGAAAGAA
AAAATCTCACGGCCATGCTCACCAGCCCCTCATGCACTTCCCTTACCCTCCAGAGCTGGGCGACCACAAACTTTAAGTCTGCAGTAGCTATCTGCCCATCCC
TTGTCAGTCGACAGCGATGAGCCCCACTAATCTAACAGCCTCTGGACTTACAACATTGTCTCTTAATGAGACCACCGCTCCTCAAGACCTGTACGATGGTGT
CTCCGCGCTGGTTAACCAGTGGGATCACCTGGGCATATGGTGGCTCCTCATAGGAGCAGTGACCCTGTGCCTAATCCTGGTCTGGATCATCTGCTGCATCAA
AAGCAGAAGACCCAGGCGGCGGCCCATCTACAGGCCCTTCGTCATCACACCTGAAGATAATGATGATGATGACACCACCTCCAGGCTGCAGAGCCTAAAGCA
GCTACTCTTCTCTTTTACAGCATGGTAAATTGAATCATGCCCCGCATTTTCATCTACTTGCTTCTCCTTCCACTTTTTCTGGGCTCCTCTACATTGGCCACT
GTGTCCCACATCGAGGTAGACTGCCTCACGCCCTTCACAGTCTACCTGCTTTTCGGCTTTGTCATCTGCACCTTTGTCTGCAGCGTTATCACTGTAGTGATC
TGCTTCATACAGTGCATCGACTACATCTGTGTGCGGGTGGCCTACTTTAGACACCACCCCCAGTATCGCAACAGGGACATAGCGGCTCTCCTAAGACTTGTT
TAAATCATGGCCAAATTACCTGTGATTGGTCTTCTGATTATCTGCTGCGTCCTAGCCGCGATTGGGACTCAACCTAATACCACCACCAGCGCTCCCAGAAAG
AGACATGTATCCTGCAGCTTCAAGCGTCCCTGGAATATACCCCAATGCTTTACTGATGAACCTGAAATCTCTTTGGCTTGGTACTTCAGCGTCACCGCCCTT
CTCATCTTCTGCAGTACGGTTATTGCTCTTGCCATCTACCCTTCCCTTAACCTGGGCTGGAATGCTGTCAACTCTATGGAATATCCCACCTTCCCAGAACCA
GACCTGCCAGACCTGGTTGTTCTAAACGCGTTTCCTCCTCCTCCAGTTCAAAATCAGTTTCGCCCTCCGTCCCCTACGCCCACTGAGGTCAGCTACTTTAAT
CTAACAGGCGGAGATGACTGAAAACCTAGACCTAGAAATGGACGGTCTCTGCAGCGAGCAACGCACACTAGAGAGGCGCCGGCAAAAAGCAGAGCTCGAGCG
TCTTAAACAAGAGCTCCAAGACGCCGTGGCCATACACCAGTGCAAAAAAGGGCTCTTCTGTCTGGTAAAACAGGCCACGCTCACCTATGAAAAAACAGGTGA
CACCCACCGCCTAGGATACAAGCTGCCCACACAGCGCCAAAAGTTTGCCCTTATGATAGGTGAACAACCCATCACCGTCACCCAGCACTCCGTGGAGACAGA
AGGCTGCATTCATGCTCCCTGCAGGGGCGCTGACTGCCTCTACACCTTGATCAAAACCCTCTGCGGTCTCAGAGACCTTATCCCTTTCAATTGATCATAACT
GTAATCAATAAAAAATCACTTACTTGAAATCTGATAGCAAGACTCTGTCCAATTTTTTCAGCAACACTTCCTTCCCCTCCTCCCAACTCTGGTACTCTAGGC
GCCTCCTAGCTGCAAACTTCCTCCACAGTCTGAAGGGAATGTCAGATTCCTCCTCCTGTCCCTCCGCACCCACGATCTTCATGTTGTTACAGATGAAACGCG
CGAGATCGTCTGACGAGACCTTCAACCCCGTGTACCCCTACGATACCGAGATCGCTCCGACTTCTGTCCCTTTCCTTACCCCTCCCTTTGTATCATCCGCAG
GAATGCAAGAAAATCCAGCTGGGGTGCTGTCCCTGCACCTGTCAGAGCCCCTTACCACCCACAATGGGGCCCTGACTCTAAAAATGGGGGGCGGCCTGACCC
TGGACAAGGAAGGGAATCTCACTTCCCAAAACATCACCAGTGTCGATCCCCCTCTCAAAAAAAGCAAGAACAACATCAGCCTTCAGACCGCCGCACCCCTCG
CCGTCAGCTCCGGGGCCCTAACCCTTTTTGCCACTCCCCCCCTAGCGGTCAGTGGCGACAACCTTACTGTGCAGTCTCAGGCCCCTCTTACTTTGGAAGACT
CAAAACTAACTCTGGCCACCAAAGGACCCCTAACTGTGTCCGAAGGCAAACTTGTCCTAGAAACAGAGCCTCCCCTGCATGCAAGTGACAGCAGTAGCCTGG
GCCTTAGCGTCACGGCCCCACTTAGCATTAACAATGACAGCCTAGGACTAGACATGCAAGCGCCCATCAGCTCTCGAGATGGAAAACTGGCTCTAACAGTGG
CGGCCCCCCTAACTGTGGCCGAGGGTATCAATGCTTTGGCAGTAGCCACAGGTAATGGTATTGGACTAAATGAAACCAACACACACCTGCAGGCAAAACTGG
TCGCGCCCCTAGGCTTTGATACCAACGGCAACATTAAGCTAAGCGTCGCAGGAGGCATGAGGCTAAACAATAACACACTGATACTAGATGTAAACTACCCAT
TTGAGGCTCAAGGCCAACTGAGCCTAAGAGTGGGCTCGGGCCCACTATATGTAGATTCTAGTAGTCATAACCTAACCATTAGATGCCTTAGGGGATTGTATG
TAACATCTTCTAACAACCAAAACGGTCTAGAGGCCAACATTAAACTAACAAAAGGCCTTGTGTATGACGGAAATGCCATAGCAGTTAATGTTGGCAAAGGGC
TGGAATACAGCCCTACTGGCACAACAGAAAAACCTATACAGACTAAAATAGGTCTAGGCATGGAGTATGACACTGAGGGAGCCATGATGACAAAACTAGGCT
CTGGACTAAGCTTTGACAATTCAGGAGCCATTGTGGTGGGAAACAAAAATGATGACAGGCTTACTTTGTGGACCACACCGGACCCATCGCCCAACTGTCAGA
TTTACTCTGAAAAAGATGCTAAACTAACCTTGGTACTGACTAAATGTGGCAGTCAGGTTGTAGGCACAGTATCTATTGCCGCTCTTAAAGGTAGCCTTGTGC
CAATCACTAGTGCAATCAGTGTGGTTCAGATATACCTAAGGTTTGATGAAAATGGGGTGCTGATGAGTAACTCTTCACTTAATGGCGAATACTGGAATTTTA
GAAACGGAGACTCAACTAATGGCACACCATATACAAACGCAGTGGGTTTTATGCCTAATCTACTGGCCTATCCTAAAGGTCAAACTACAACTGCAAAAAGTA
ACATTGTCAGCCAGGTCTACATGAACGGGGACGATACTAAACCCATGACATTTACAATCAACTTCAATGGCCTTAGTGAAACAGGGGATACCCCTGTCAGTA
AATATTCCATGACATTCTCATGGAGGTGGCCAAATGGAAGCTACATAGGGCACAATTTTGTAACAAACTCCTTTACTTTCTCCTACATCGCCCAAGAATAAA
GAAAGCACAGAGATGCTTGTTTTTGATTTCAAAATTGTGTGCTTTTATTTATTTTCAAGCTTACAGTATTTCCAGTAGTCATTAGAATAGAGCTTAATTAAA
CTGCATGAGAACCCTTCCACATAGCTTAAATTATCACCAGTGCAAATGGAAAAAAATCAACATACCTTTTTATCCAGATATCAAAGAACTCTAGTGGTCAGT
TTTCCCCCACCCTCCCAGCTCACAGAATACACAGTCCTTTCCCCCCGGCTGGCTTTAAACAACACTATCTCATTGGTAACAGACATATTTTTAGGTGTAATA
ATCCACACGGTCTCTTGGCGGGCCAAACGCTGGTCTGTGATGTTAATAAACTCCCCAGGCAGCTCTTTCAAGTTCACGTCGCTGTCCAACTGCTGAAGCGCT
CGCGGCTCCGACTGCGCCTCTAGCGGAGGCAACGGCAGCACCCGATCCTTGATCTATAAAGGAGTAGAGTCATAATCCCCCATAAGAATAGGGCGGTGATGC
AGCAACAAGGCGCGCAGCAACTCCTGCCGCCGCCTCTCCGTACGACAGGAATGCAACGGGGTGGTGGTCTCCTCCGCGATAATCCGCACCGCTCGCAGCATC
AGCATCCTCGTCCTCCGGGCACAGCAGCGCATCCTGATCTCACTGAGATCGGCGCAGTAAGTGCAGCACAACACCAAGATGTTATTTAAGATCCCACAGTGC
AAAGCACTGTACCCAAAGCTCATGGCGGGAAGGACAGCCCCCACGTGACCATCGTACCAGATCCTCAGGTAAATCAAATGACGACCTCTCATAAACACGCTG
GACATATACATCACCTCCTTGGGCATGAGCTGATTCACCACCTCTCGATACCACAGGCATCGCTGATTAATTAAAGACCCCTCGAGCACCATCCTGAACCAG
GAAGCCAGCACCTGACCCCCCGCCAGGCACTGCAGGGACCCCGGTGAATCGCAGTGGCAGTGAAGACTCCAGCGCTCGTAGCCGTGAACCATAGAGCTGGTC
ATTATATCCACATTGGCACAACACAGACACACTTTCATACACTTTTTCATGATTAGCAGCTCCTCTCTAGTCAAGACCATATCCCAAGGAATCACCCACTCT
TGAATCAAGGTAAATCCCACACAGCAGGGCAGGCCTCTCACATAACTCACGTTATGCATAGTGAGCGTGTCGCAATCTGGAAATACCGGATGATCTTCCATC
ACCGAAGCCCGGGTCTCCGTCTCAAAGGGAGGTAAACGGTCCCTCGTGTAGGGACAGTGGCGGGATAATCGAGATCGTGTTGAACGTAGAGTCATGCCAAAG
GGAACAGCGGACGTACTCATATTTCCTCCAGCAGAACCAAGTGCGCGCGTGGCAGCTATCCCTGCGTCTTCTGTCTCGCCGCCTGCCCCGCTCGGTGTAGTA
GTTGTAATACAGCCACTCCCTCAGACCGTCAAGGCGCTCCCTGGCGTCCGGATCTATAACAACACCGTCCTGCAGCGCCGCCCTGATGACATCCACCACCGT
AGAGTATGCCAAGCCCAGCCACGAAATGCACTCACTTTGACAGCGAGAGATAGGAGGAGCGGGAAGAGATGGAAGAACCATGATAGTAAAAGAACTTTTATT
CCAATCGATCCTCTACAATGTCAAAGTGTAGATCTATCAGATGGCACTGGTCTCCTCCGCTGAGTCGATCAAAAATAACAGCTAAACCACAAACAACACGAT
TGGTCAAATGCTGCACAAGGGCTTGCAGCATAAAATCGCCTCGAAAGTCCACCGCAAGCATAACATCAAAGCCACCGCCCCTATCATGATCTATGATAAAAA
CCCCACAGCTATCCACCAGACCCATATAGTTTTCATCTCTCCATCGTGAAAAAATATTTACAAGCTCCTCCTTTAAATCACCTCCAACCAATTCAAAAAGTT
GAGCCAGACCGCCCTCCACCTTCATTTTCAGCATGCGCATCATGATTGCAAAAATTCAGGCTCCTCAGACACCTGTATAAGATTGAGAAGCGGAACGTTAAC
ATCAATGTTTCGCTCGCGAAGATCGCGCCTCAGTGCAAGCATGATATAATCCCACAGGTCGGAGCGGATCAGCGAGGACATCTCCCCGCCAGGAACCAACTC
AACGGAGCCTATGCTGATTATAATACGCATATTCGGGGCTATGCTAACCAGCACGGCCCCCAAATAGGCGTACTGCATAGGCGGCGACAAAAAGTGAACAGT
TTGGGTTAAAAAATCAGGCAAACACTCGCGCAAAAAAGCAAGAACATCATAACCATGCTCATGCAAATAGATGCAAGTAAGCTCAGGAACGACCACAGAAAA
ATGCACAATTTTTCTCTCAAACATGACTGCGAGCCCTGCAAAAAATAAAAAAGAAACATTACACAAGAGTAGCCTGTCTTACAATGGGATAGACTACTCTAA
CCAACATAAGACGGGCCACGACATCGCCCGCGTGGCCATAAAAAAAATTATCCGTGTGATTAAAAAGAAGCACAGATAGCTGGCCAGTCATATCCGGAGTCA
TCACGTGCGAACCCGTGTAGACCCCCGGGTTGGACACATCGGCCAAACAAAGAAAGCGGCCAATGTATCCCGGAGGAATGATAACACTAAGACGAAGATACA
ACAGAATAACCCCATGGGGGGGAATAACAAAGTTAGTAGGTGAATAAAAACGATAAACACCCGAAACTCCCTCCTGCGTAGGCAAAATAGCGCCCTCCCCTT
CCAAAACAACATACAGCGCTTCCACAGCAGCCATGACAAAAGACTCAAAACACTCAAAAGACTCAGTCTTACCAGGAAAATAAAAGCACTCTCACAGCACCA
GCACTAATCAGAGTGTGAAGAGGGCCAAGTGCCGAACGAGTATATATAGGAATTAAAAATGACGTAAATGTGTAAAGGTCAAAAAACGCCCAGAAAAATACA
CAGACCAACGCCCGAAACGAAAACCCGCGAAAAAATACCCAGAAGTTCCTCAACAACCGCCACTTCCGCTTTCCCACGATACGTCACTTCCTCAAAAATAGC
AAACTACATTTCCCACATGTACAAAACCAAAACCCCTCCCCTTGTCACCGCCCACAACTTACATAATCACAAACGTCAAAGCCTACGTCACCCGCCCCGCCT
CGCCCCGCCCACCTCATTATCATATTGGCCTCAATCCAAAATAAGGTATATTATTGATGATG

### EMBODIMENTS

The following list of embodiments is intended to complement, rather than displace or supersede, the previous descriptions.

Embodiment 1. A method of diagnosing a subject with prostate cancer, the method comprising:

evaluating the presence of one or more prostate cancer neoantigens in a sample from the subject, the one or more prostate cancer neoantigens comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof,

wherein the presence of the one or more prostate cancer neoantigens is indicative of prostate cancer in the subject.

Embodiment 2. The method of embodiment 1, wherein the one or more prostate cancer neoantigens comprise the amino acid sequence of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, fragments of the preceding sequences, or any combination thereof.

Embodiment 3. The method of embodiment 1 or 2, wherein the one or more prostate cancer neoantigens comprise the amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, 223, fragments of the preceding sequences, or any combination thereof.

Embodiment 4. The method of embodiment 3, wherein the method comprises evaluating the presence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, and 223.

Embodiment 5. The method of any one of the previous embodiments, wherein the presence of the one or more prostate cancer neoantigens is evaluated by qPCR.

Embodiment 6. The method of embodiment 5, further comprising, prior to performing the qPCR, extracting RNA from the sample from the subject and synthesizing cDNA from the extracted RNA.

Embodiment 7. The method of embodiment 6, wherein the RNA is produced from a DNA sequence comprising SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 380, 382, 384, 386, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 503, 504, 505, 506, 507, 508, 509, 510, 511, 512, 513, 514, 515, 516, 517, 519, 520, 521, 522, 523, 524, 525, 528, 529, 530, 531, 532, 533, 534, 535, 536, 537, 538, 539, 540, fragments of the preceding sequences, or any combination thereof.

Embodiment 8. The method of any one of the previous embodiments, wherein the sample comprises a prostate cancer tissue sample.

Embodiment 9. The method of any one of the previous embodiments, wherein the prostate cancer is a localized prostate adenocarcinoma, a relapsed prostate cancer, a refractory prostate cancer, a metastatic prostate cancer, a castration resistant prostate cancer, or any combination thereof.

Embodiment 10. The method of any one of the previous embodiments, wherein the subject is treatment naïve.

Embodiment 11. The method of any one of embodiments 1-9, wherein the subject has received androgen deprivation therapy.

Embodiment 12. The method of any one of the previous embodiments, wherein the subject has an elevated level of prostate specific antigen (PSA).

Embodiment 13. A method of treating prostate cancer in a subject, the method comprising:
a) evaluating the presence of one or more prostate cancer neoantigens in a sample from the subject, the one or more prostate cancer neoantigens comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof; and
b) administering a therapeutically effective amount of a prostate cancer vaccine to the subject to thereby treat the prostate cancer.

Embodiment 14. The method of embodiment 13, wherein the one or more prostate cancer neoantigens comprise the amino acid sequence of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, fragments of the preceding sequences, or any combination thereof.

Embodiment 15. The method of embodiment 13 or 14, wherein the one or more prostate cancer neoantigens comprise the amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, 223, fragments of the preceding sequences, or any combination thereof.

Embodiment 16. The method of embodiment 15, wherein the method comprises evaluating the presence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, and 223.

Embodiment 17. The method of any one of embodiments 13-16, wherein the presence of the one or more prostate cancer neoantigens is evaluated by qPCR.

Embodiment 18. The method of embodiment 17, further comprising, prior to performing the qPCR, extracting RNA from the sample from the subject and synthesizing cDNA from the extracted RNA.

Embodiment 19. The method of any one of embodiments 13-18, wherein the sample comprises a prostate cancer tissue sample.

Embodiment 20. The method of any one of embodiments 13-19, wherein the prostate cancer vaccine comprises a polynucleotide encoding one or more polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, and fragments thereof.

Embodiment 21. The method of embodiment 20, wherein the polynucleotide comprises:
a) one or more polynucleotides selected from the group consisting of SEQ ID NOs: 276, 382, 334, 338, 270, 254, 310, 326, 272, 306, 252, 246, 262, 266, 318, 256, 278, 298, 286, 448, 450, 453, 455, 380, 344, 212, 350, 214, 216, 222, 220, 226, 346, 354, 236, 224, 168, 172, 20, 24, 178, and fragments thereof;
b) one or more polynucleotides selected from the group consisting of SEQ ID NOs: 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and fragments thereof; or
c) one or more polynucleotides selected from the group consisting of SEQ ID NOs: 500, 501, 461, 503, 504, 505, 506, 507, 508, 509, 510, 511, 512, 513, 514, 515, 516, 517, 477, 519, 520, 521, 522, 523, 524, 525, 485, 486, 528, 529, 530, 531, 532, 533, 534, 535, 536, 537, 538, 539, 540, and fragments thereof.

Embodiment 22. The method of any one of embodiments 13-21, wherein the polynucleotide encodes a polypeptide comprising the amino acid sequence of SEQ ID NOs: 541, 550, 554, 555, 556, 623, 624, 543, 552, 557, 558, 559, 625, or 626.

Embodiment 23. The method of embodiment 22, wherein the polynucleotide comprises a polynucleotide sequence of SEQ ID NOs: 542, 551, 544, or 553.

Embodiment 24. The method of any one of embodiments 13-23, wherein the polynucleotide is DNA or RNA.

Embodiment 25. The method of embodiment 24, wherein RNA is mRNA or self-replicating RNA.

Embodiment 26. The method of embodiment 13-25, wherein the vaccine is a recombinant virus.

Embodiment 27. The method of embodiment 26, wherein the recombinant virus is derived from an adenovirus (Ad), a poxvirus, an adeno-associated virus (AAV), or a retrovirus.

Embodiment 28. The method of embodiment 27, wherein the recombinant virus is derived from hAd5, hAd7, hAd11, hAd26, hAd34, hAd35, hAd48, hAd49, hAd50, GAd20, GAd19, GAd21, GAd25, GAd26, GAd27, GAd28, GAd29, GAd30, GAd31, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAd1, PanAd2, PanAd3, Copenhagen vaccinia virus (W), New York Attenuated Vaccinia Virus (NYVAC), ALVAC, TROVAC, or modified vaccinia ankara (MVA).

Embodiment 29. The method of embodiment 28, wherein the recombinant virus is derived from GAd20.

Embodiment 30. The method of embodiment 28, wherein the recombinant virus is derived from MVA.

Embodiment 31. The method of embodiment 28, wherein the recombinant virus is derived from hAd26.

Embodiment 32. The method of embodiment 29 or 31, wherein the polynucleotide encodes a polypeptide of SEQ ID NOs: 541, 550, 554, 555, 556, 623, or 624.

Embodiment 33. The method of embodiment 30 or 31, wherein the polynucleotide encodes a polypeptide of SEQ ID NOs: 543, 552, 557, 558, 559, 625, or 626.

Embodiment 34. The method of any one of embodiments 13-33, wherein the prostate cancer is a localized prostate adenocarcinoma, a relapsed prostate cancer, a refractory prostate cancer, a metastatic prostate cancer or a castration resistant prostate cancer, or any combination thereof.

Embodiment 35. The method of any one of embodiments 13-34, wherein the subject is treatment naïve.

Embodiment 36. The method of any one of embodiments 13-34, wherein the subject has received androgen deprivation therapy.

Embodiment 37. The method of any one of embodiments 13-36, wherein the subject has an elevated level of prostate specific antigen (PSA).

Embodiment 38. A method of treating prostate cancer in a subject, the method comprising:
a) evaluating the presence of one or more prostate cancer neoantigens in a sample from the subject, the one or more prostate cancer neoantigens comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof; and;
b) evaluating expression of one or more prostate cancer biomarkers in a sample from the subject, wherein the one or more prostate cancer biomarkers comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, ROR1, FGF8, NKX2-2, EDIL3, RELN, FGF9, AKR1C4, CLUL1, KISSIR, CYP3A5, CYP17A1, SFRP4, HNF1A, CALCR, SYP, MSLN, or any combination thereof; and
c) administering a therapeutically effective amount of a prostate cancer vaccine to the subject.

Embodiment 39. The method of embodiment 38, further comprising, after administering the therapeutically effective amount of the prostate cancer vaccine, evaluating expression of the one or more prostate cancer biomarkers evaluated in step b), wherein a decrease in expression compared to the expression in step b) is indicative of responsiveness to the prostate cancer vaccine.

Embodiment 40. The method of embodiment 38 or 39, wherein the one or more prostate cancer neoantigens comprise the amino acid sequence of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, fragments of the preceding sequences, or any combination thereof.

Embodiment 41. The method of any one of embodiments 38-40, wherein the one or more prostate cancer neoantigens comprise the amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, 223, fragments of the preceding sequences, or any combination thereof.

Embodiment 42. The method of embodiment 41, wherein the method comprises evaluating the presence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, and 223.

Embodiment 43. The method of any one of embodiments 38-42, wherein the presence of the one or more prostate cancer neoantigens is evaluated by qPCR.

Embodiment 44. The method of embodiment 43, further comprising, prior to performing the qPCR, extracting RNA from the sample from the subject and synthesizing cDNA from the extracted RNA.

Embodiment 45. The method of any one of embodiments 38-44, wherein the one or more neoantigens are from a prostate cancer tissue sample.

Embodiment 46. The method of any one of embodiments 38-45, wherein the one or more prostate cancer biomarkers comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, or combinations thereof.

Embodiment 47. The method of any one of embodiments 38-45, wherein the one or more prostate cancer biomarkers comprise: HPN, ROR1, FLNC, GPR39, FGF8, NKX2-2, MUC1, NKX3-1, EDIL3, LGR5, FGFR4, STEAP1, ATF3, RELN, UGT2B17, KLK3, C9orf152, GNMT, METTL7A, FGF9, SPDEF, FOXA1, AKR1C4, GREB1, CLUL1, TMEFF2, HOXB13, KLK2, NPY, GRHL2, STEAP2, THBS2, KISSIR, KRT8, TNFRSF19, CYP3A5, KLK4, IDO1, FOLH1, NROB1, EPHA3, CYP17A1, SFRP4, KRT18, TSPAN1, HNF1A, ADAMTS15, ACPP, CALCR, SYP, AZGP1, AR, ARv3, MSLN, TMPRSS2:ERG, and combinations thereof.

Embodiment 48. The method of embodiment 46, wherein the one or more prostate cancer biomarkers are from a plasma sample.

Embodiment 49. The method of embodiment 47, wherein the one or more prostate cancer biomarkers are from a blood sample.

Embodiment 50. The method of any one of embodiments 38-49, wherein the expression of the one or more prostate cancer biomarkers is evaluated by qPCR.

Embodiment 51. The method of embodiment 50, further comprising, prior to performing the qPCR, extracting RNA from the sample from the subject and synthesizing cDNA from the extracted RNA.

Embodiment 52. The method of any one of embodiments 38-51, wherein the prostate cancer vaccine comprises a polynucleotide encoding one or more polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, and fragments thereof.

Embodiment 53. The method of embodiment 52, wherein the polynucleotide comprises:
a) one or more polynucleotides selected from the group consisting of SEQ ID NOs: 276, 382, 334, 338, 270, 254, 310, 326, 272, 306, 252, 246, 262, 266, 318, 256, 278, 298, 286, 448, 450, 453, 455, 380, 344, 212, 350, 214, 216, 222, 220, 226, 346, 354, 236, 224, 168, 172, 20, 24, 178, and fragments thereof;
b) one or more polynucleotides selected from the group consisting of SEQ ID NOs: 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and fragments thereof; or
c) one or more polynucleotides selected from the group consisting of SEQ ID NOs: 500, 501, 461, 503, 504, 505, 506, 507, 508, 509, 510, 511, 512, 513, 514, 515, 516, 517, 477, 519, 520, 521, 522, 523, 524, 525, 485, 486, 528, 529, 530, 531, 532, 533, 534, 535, 536, 537, 538, 539, 540, and fragments thereof.

Embodiment 54. The method of embodiment 52 or 53, wherein the polypeptide comprises the amino acid sequence of SEQ ID NOs: 541, 550, 554, 555, 556, 623, 624, 543, 552, 557, 558, 559, 625, or 626.

Embodiment 55. The method of embodiment 54, wherein the polynucleotide comprises a polynucleotide sequence of SEQ ID NOs: 542, 551, 544 or 553.

Embodiment 56. The method of any one of embodiments 52-55, wherein the polynucleotide is DNA or RNA.

Embodiment 57. The method of embodiment 56, wherein RNA is mRNA or self-replicating RNA.

Embodiment 58. The method of any one of embodiments 38-57, wherein the vaccine is a recombinant virus.

Embodiment 59. The method of embodiment 58, wherein the recombinant virus is derived from an adenovirus (Ad), a poxvirus, an adeno-associated virus (AAV), or a retrovirus.

Embodiment 60. The method of embodiment 59, wherein the recombinant virus is derived from hAd5, hAd7, hAd11, hAd26, hAd34, hAd35, hAd48, hAd49, hAd50, GAd20, GAd19, GAd21, GAd25, GAd26, GAd27, GAd28, GAd29, GAd30, GAd31, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAd1, PanAd2, PanAd3, Copenhagen vaccinia virus (W), New York Attenuated Vaccinia Virus (NYVAC), ALVAC, TROVAC, or modified vaccinia ankara (MVA).

Embodiment 61. The method of embodiment 60, wherein the recombinant virus is derived from GAd20.

Embodiment 62. The method of embodiment 60, wherein the recombinant virus is derived from MVA.

Embodiment 63. The method of embodiment 60, wherein the recombinant virus is derived from hAd26.

Embodiment 64. The method of embodiment 61 or 63, wherein the polynucleotide encodes a polypeptide of SEQ ID NOs: 541, 550, 554, 555, 556, 623, or 624.

Embodiment 65. The method of embodiment 62 or 63, wherein the polynucleotide encodes a polypeptide of SEQ ID NOs: 543, 552, 557, 558, 559, 625, or 626.

Embodiment 66. The method of any one of embodiments 38-65, wherein the prostate cancer is a localized prostate adenocarcinoma, a relapsed prostate cancer, a refractory prostate cancer, a metastatic prostate cancer, a castration resistant prostate cancer, or any combination thereof.

Embodiment 67. The method of any one of embodiments 38-66, wherein the subject is treatment naïve.

Embodiment 68. The method of any one of embodiments 38-66, wherein the subject has received androgen deprivation therapy.

Embodiment 69. The method of any one of embodiments 38-68, wherein the subject has an elevated level of prostate specific antigen (PSA).

Embodiment 70. A method for monitoring responsiveness of a subject having prostate cancer to a therapeutic agent, the method comprising:

(a) evaluating expression of one or more prostate cancer biomarkers, wherein the one or more prostate cancer biomarkers comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, ROR1, FGF8, NKX2-2, EDIL3, RELN, FGF9, AKR1C4, CLUL1, KISSIR, CYP3A5, CYP17A1, SFRP4, HNF1A, CALCR, SYP, MSLN, or combinations thereof;

(b) administering a therapeutic agent to the subject; and

(c) evaluating the expression of the one or more prostate cancer biomarkers evaluated in step (a), wherein a decrease in the expression of the one or more prostate cancer biomarkers compared to the expression in step (a) is indicative of responsiveness to the therapeutic agent.

Embodiment 71. The method of embodiment 70, wherein the one or more prostate cancer biomarkers comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, or combinations thereof.

Embodiment 72. The method of embodiment 70, wherein the one or more prostate cancer biomarkers comprise: HPN, ROR1, FLNC, GPR39, FGF8, NKX2-2, MUC1, NKX3-1, EDIL3, LGR5, FGFR4, STEAP1, ATF3, RELN, UGT2B17, KLK3, C9orf152, GNMT, METTL7A, FGF9, SPDEF, FOXA1, AKR1C4, GREB1, CLUL1, TMEFF2, HOXB13, KLK2, NPY, GRHL2, STEAP2, THBS2, KISSIR, KRT8, TNFRSF19, CYP3A5, KLK4, IDO1, FOLH1, NROB1, EPHA3, CYP17A1, SFRP4, KRT18, TSPAN1, HNF1A, ADAMTS15, ACPP, CALCR, SYP, AZGP1, AR, ARv3, MSLN, TMPRSS2:ERG, and combinations thereof.

Embodiment 73. The method of embodiment 71, wherein the one or more prostate cancer biomarkers are from a plasma sample.

Embodiment 74. The method of embodiment 72, wherein the one or more prostate cancer biomarkers are from a blood sample.

Embodiment 75. The method of any one of embodiments 70-74, wherein the expression of the one or more prostate cancer biomarkers is evaluated by qPCR.

Embodiment 76. The method of embodiment 75, further comprising, prior to performing the qPCR, extracting RNA from the sample from the subject and synthesizing cDNA from the extracted RNA.

Embodiment 77. A method for preparing a cDNA from a subject with prostate cancer useful for analyzing an expression of prostate cancer neoantigens, the method comprising:
(a) extracting RNA from a sample from the subject;
(b) producing amplified cDNA from the RNA extracted in step (a) by:
   (i) reverse transcribing the extracted RNA to produce the cDNA, and
   (ii) amplifying the cDNA; and
(c) analyzing the amplified cDNA produced in step (b) for one or more prostate cancer neoantigens, wherein the cDNA encodes an amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof.

Embodiment 78. The method of embodiment 77, wherein the cDNA encodes an amino acid sequence of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, fragments of the preceding sequences, or any combination thereof.

Embodiment 79. The method of embodiment 77 or 78, wherein the cDNA encodes an amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, 223, fragments of the preceding sequences, or any combination thereof.

Embodiment 80. The method of embodiment 79, wherein the cDNA encodes an amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, and 223.

Embodiment 81. A method of treating prostate cancer in a subject, the method comprising: administering a therapeutically effective amount of a prostate cancer vaccine to the subject to thereby treat the prostate cancer, wherein the prostate cancer vaccine comprises a polynucleotide encoding one or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof.

Embodiment 82. The method of embodiment 81, wherein the prostate cancer vaccine comprises a polynucleotide encoding one or more polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, and fragments thereof.

Embodiment 83. The method of embodiment 81 or 82, wherein the polynucleotide comprises:
a) one or more polynucleotides selected from the group consisting of SEQ ID NOs: 276, 382, 334, 338, 270, 254, 310, 326, 272, 306, 252, 246, 262, 266, 318, 256, 278, 298, 286, 448, 450, 453, 455, 380, 344, 212, 350, 214, 216, 222, 220, 226, 346, 354, 236, 224, 168, 172, 20, 24, 178, and fragments thereof;
b) one or more polynucleotides selected from the group consisting of SEQ ID NOs: 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and fragments thereof; or
c) one or more polynucleotides selected from the group consisting of SEQ ID NOs: 500, 501, 461, 503, 504, 505, 506, 507, 508, 509, 510, 511, 512, 513, 514, 515, 516, 517, 477, 519, 520, 521, 522, 523, 524, 525, 485, 486, 528, 529, 530, 531, 532, 533, 534, 535, 536, 537, 538, 539, 540, and fragments thereof.

Embodiment 84. The method of any one of embodiments 81-83, wherein the polynucleotide encodes a polypeptide comprising the amino acid sequence of SEQ ID NOs: 541, 550, 554, 555, 556, 623, 624, 543, 552, 557, 558, 559, 625, or 626.

Embodiment 85. The method of embodiment 84, wherein the polynucleotide comprises the sequence of SEQ ID NOs: 542, 551, 544, or 553.

Embodiment 86. The method of any one of embodiments 81-85, wherein the polynucleotide is DNA or RNA.

Embodiment 87. The method of embodiment 86, wherein RNA is mRNA or self-replicating RNA.

Embodiment 88. The method of any one of embodiments 81-87, wherein the vaccine is a recombinant virus.

Embodiment 89. The method of embodiment 88, wherein the recombinant virus is derived from an adenovirus (Ad), a poxvirus, an adeno-associated virus (AAV), or a retrovirus.

Embodiment 90. The method of embodiment 89, wherein the recombinant virus is derived from hAd5, hAd7, hAd11, hAd26, hAd34, hAd35, hAd48, hAd49, hAd50, GAd20, GAd19, GAd21, GAd25, GAd26, GAd27, GAd28, GAd29, GAd30, GAd31, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAd1, PanAd2, PanAd3, Copenhagen vaccinia virus (W), New York Attenuated Vaccinia Virus (NYVAC), ALVAC, TROVAC, or modified vaccinia ankara (MVA).

Embodiment 91. The method of embodiment 89, wherein the recombinant virus is derived from GAd20.

Embodiment 92. The method of embodiment 89, wherein the recombinant virus is derived from MVA.

Embodiment 93. The method of embodiment 89, wherein the recombinant virus is derived from hAd26.

Embodiment 94. The method of embodiment 91 or 93, wherein the polynucleotide encodes a polypeptide of SEQ ID NOs: 541, 550, 554, 555, 556, 623, or 624.

Embodiment 95. The method of embodiment 92 or 93, wherein the polynucleotide encodes a polypeptide of SEQ ID NOs: 543, 552, 557, 558, 559, 625, or 626.

Embodiment 96. The method of any one of embodiments 81-95, wherein the prostate cancer is a localized prostate adenocarcinoma, a relapsed prostate cancer, a refractory prostate cancer, a metastatic prostate cancer or a castration resistant prostate cancer, or any combination thereof.

Embodiment 97. The method of any one of embodiments 81-96, wherein the subject is treatment naïve.

Embodiment 98. The method of any one of embodiments 81-96, wherein the subject has received androgen deprivation therapy.

Embodiment 99. The method of any one of embodiments 81-98, wherein the subject has an elevated level of prostate specific antigen (PSA). cm 1-12. (canceled)

13. A method of treating prostate cancer in a subject, the method comprising:
in a subject having a presence of one or more prostate cancer neoantigens comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof, in a sample from the subject,
administering a therapeutically effective amount of a prostate cancer vaccine to the subject to thereby treat the prostate cancer.

14. The method of claim 13, wherein the one or more prostate cancer neoantigens comprise the amino acid sequence of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, fragments of the preceding sequences, or any combination thereof.

15. The method of claim 13, wherein the one or more prostate cancer neoantigens comprise the amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, 223, fragments of the preceding sequences, or any combination thereof.

16. (canceled)

17. The method of claim 13, wherein the presence of the one or more prostate cancer neoantigens is evaluated by qPCR.

18. The method of claim 17, further comprising, prior to performing the qPCR, extracting RNA from the sample from the subject and synthesizing cDNA from the extracted RNA.

19. The method of claim 13, wherein the sample comprises a prostate cancer tissue sample.

20. The method of claim 13, wherein the prostate cancer vaccine comprises a polynucleotide encoding one or more polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, and fragments thereof.

21. The method of claim 20, wherein the prostate cancer vaccine comprises a polynucleotide comprising:
a) one or more polynucleotides selected from the group consisting of SEQ ID NOs: 276, 382, 334, 338, 270, 254, 310, 326, 272, 306, 252, 246, 262, 266, 318, 256, 278, 298, 286, 448, 450, 453, 455, 380, 344, 212, 350, 214, 216, 222, 220, 226, 346, 354, 236, 224, 168, 172, 20, 24, 178, and fragments thereof,
b) one or more polynucleotides selected from the group consisting of SEQ ID NOs: 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and fragments thereof, or
c) one or more polynucleotides selected from the group consisting of SEQ ID NOs: 500, 501, 461, 503, 504, 505, 506, 507, 508, 509, 510, 511, 512, 513, 514, 515, 516, 517, 477, 519, 520, 521, 522, 523, 524, 525, 485, 486, 528, 529, 530, 531, 532, 533, 534, 535, 536, 537, 538, 539, 540, and fragments thereof.

22. The method of claim 20, wherein the prostate cancer vaccine comprises a polynucleotide encoding a polypeptide comprising the amino acid sequence of SEQ ID NOs: 541, 550, 554, 555, 556, 623, 624, 543, 552, 557, 558, 559, 625, or 626.

23. The method of claim 22, wherein the polynucleotide comprises a polynucleotide sequence of SEQ ID NOs: 542, 551, 544, or 553.

24. The method of claim 20, wherein the polynucleotide is DNA or RNA.

25. The method of claim 24, wherein RNA is mRNA or self-replicating RNA.

26. The method of claim 20, wherein the vaccine is a recombinant virus.

27. The method of claim 26, wherein the recombinant virus is derived from an adenovirus (Ad), a poxvirus, an adeno-associated virus (AAV), or a retrovirus.

28. (canceled)

29. The method of claim 27, wherein the recombinant virus is derived from GAd20, MVA, or hAd26.

30-31. (canceled)

32. The method of claim 29, wherein the recombinant virus is derived from GAd20 and comprises a polynucleotide encoding a polypeptide of SEQ ID NOs: 541, 550, 554, 555, 556, 623, or 624.

33. The method of claim 29, wherein the recombinant virus is derived from MVA and comprises a polynucleotide encoding a polypeptide of SEQ ID NOs: 543, 552, 557, 558, 559, 625, or 626.

34-37. (canceled)

38. The method of claim 13, further comprising,
evaluating expression of one or more prostate cancer biomarkers in a sample from the subject, wherein the one or more prostate cancer biomarkers comprise: RCN1, STEAP1, PITX2, TIMP1, KLK4, KRT18, KLK3, ACPP, KLK2, TSPAN1, ATF3, SPDEF, NPY, SPINK1, HOXB13, FOXA1, KRT17, FOLH1, TNFRSF19, GREB1, KRT8, FLNC, GRHL2, RAB3B, JCHAIN, TMEFF2, AGR2, ACADL, AZGP1, MUC1, STEAP2, UGT2B17, METTL7A, HPN, NKX3-1, GNMT, ADAMTS15, HSD3B2, EPHA3, KCNN2, LGR5, IDO1, GPR39, C9orf152, MYBPC1, THBS2, ETV7, COL1A1, FGFR4, NROB1, AR, ARv3, TMPRSS2:ERG, ROR1, FGF8, NKX2-2, EDIL3, RELN, FGF9, AKR1C4, CLUL1, KISS1R, CYP3A5, CYP17A1, SFRP4, HNF1A, CALCR, SYP, MSLN, or any combination thereof.

39. The method of claim 38, wherein the expression of the one or more prostate cancer biomarkers are evaluated before and after administering the therapeutically effective amount of the prostate cancer vaccine, and wherein a decrease in expression of the one or more prostate cancer biomarkers after administration of the prostate cancer vaccine is indicative of responsiveness to the prostate cancer vaccine.

40-49. (canceled)

50. The method of claim 38, wherein the expression of the one or more prostate cancer biomarkers is evaluated by qPCR.

51. The method of claim 50, further comprising, prior to performing the qPCR, extracting RNA from the sample from the subject and synthesizing cDNA from the extracted RNA.

52-76. (canceled)

77. A method for preparing a cDNA from a subject with prostate cancer useful for analyzing an expression of prostate cancer neoantigens, the method comprising:
(a) extracting RNA from a sample from the subject;
(b) producing amplified cDNA from the RNA extracted in step (a) by:
(i) reverse transcribing the extracted RNA to produce the cDNA, and
(ii) amplifying the cDNA; and
(c) analyzing the amplified cDNA produced in step (b) for one or more prostate cancer neoantigens, wherein the cDNA encodes an amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof.

78. The method of claim 77, wherein the cDNA encodes an amino acid sequence of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, fragments of the preceding sequences, or any combination thereof.

79. The method of claim 77, wherein the cDNA encodes an amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, 223, fragments of the preceding sequences, or any combination thereof.

80. The method of claim 79, wherein the cDNA encodes an amino acid sequence of SEQ ID NOs: 275, 381, 333, 337, 269, 253, 309, 325, 271, 305, 251, 245, 261, 265, 317, 255, 297, 285, 437, 439, 442, 444, 379, 343, 211, 349, 213, 215, 221, 219, 225, 345, 353, 235, and 223.

81. A method of treating prostate cancer in a subject, the method comprising:
administering a therapeutically effective amount of a prostate cancer vaccine to the subject to thereby treat the prostate cancer, wherein the prostate cancer vaccine comprises a polynucleotide encoding one or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 388, 389, 390, 391, 392, 393, 394, 395, 396, 397, 398, 399, 400, 401, 402, 403, 404, 405, 406, 407, 408, 426, 427, 428, 429, 430, 431, 432, 433, 434, 435, 436, 437, 438, 439, 440, 441, 442, 443, 444, 445, 446, 447, fragments of the preceding sequences, or any combination thereof.

82. The method of claim 81, wherein the prostate cancer vaccine comprises a polynucleotide encoding one or more polypeptides selected from the group consisting of SEQ ID NOs: 275, 381, 333, 337, 269, 253, 309, 325, 271, 305, 251, 245, 261, 265, 317, 255, 277, 297, 285, 437, 439, 442, 444, 379, 343, 211, 349, 213, 215, 221, 219, 225, 345, 353, 235, 223, 167, 171, 19, 23, 177, and fragments thereof.

83. The method of claim 81, wherein the polynucleotide comprises:
a) one or more polynucleotides selected from the group consisting of SEQ ID NOs: 276, 382, 334, 338, 270, 254, 310, 326, 272, 306, 252, 246, 262, 266, 318, 256, 278, 298, 286, 448, 450, 453, 455, 380, 344, 212, 350, 214, 216, 222, 220, 226, 346, 354, 236, 224, 168, 172, 20, 24, 178, and fragments thereof,
b) one or more polynucleotides selected from the group consisting of SEQ ID NOs: 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and fragments thereof, or
c) one or more polynucleotides selected from the group consisting of SEQ ID NOs: 500, 501, 461, 503, 504, 505, 506, 507, 508, 509, 510, 511, 512, 513, 514, 515, 516, 517, 477, 519, 520, 521, 522, 523, 524, 525, 485, 486, 528, 529, 530, 531, 532, 533, 534, 535, 536, 537, 538, 539, 540, and fragments thereof.

84. The method of claim 81, wherein the polynucleotide encodes a polypeptide comprising the amino acid sequence of SEQ ID NOs: 541, 550, 554, 555, 556, 623, 624, 543, 552, 557, 558, 559, 625, or 626.

85. The method of claim 84, wherein the polynucleotide comprises the sequence of SEQ ID NOs: 542, 551, 544, or 553.

86. The method of claim 81, wherein the polynucleotide is DNA or RNA.

87. The method of claim 86, wherein RNA is mRNA or self-replicating RNA.

88. The method of claim 81, wherein the vaccine is a recombinant virus.

89. The method of claim 88, wherein the recombinant virus is derived from an adenovirus (Ad), a poxvirus, an adeno-associated virus (AAV), or a retrovirus.

90. (canceled)

91. The method of claim 89, wherein the recombinant virus is derived from GAd20, MVA, or hAd26.

92-93. (canceled)

94. The method of claim 91, wherein the polynucleotide encodes a polypeptide of SEQ ID NOs: 541, 550, 554, 555, 556, 623, or 624.

95. The method of claim 91, wherein the polynucleotide encodes a polypeptide of SEQ ID NOs: 543, 552, 557, 558, 559, 625, or 626.

96-99. (canceled)
